(19) United States
(12) Patent Application Publication
(10) Pub. No.: US 2024/0368163 A1
(43) Pub. Date: Nov. 7, 2024

(54) COMPOUNDS AND METHODS FOR MODULATING SPLICING

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Sudeep Prajapati, Somerville, MA (US); Allen T. Hopper, Lexington, MA (US); Stepan Vyskocil, Benesov (CZ)

(21) Appl. No.: 18/688,098

(22) PCT Filed: Aug. 30, 2022
(86) PCT No.: PCT/US2022/075702

§ 371 Date: Feb. 29, 2024

Related U.S. Application Data
(60) Provisional application No. 63/238,409, filed on Aug. 30, 2021.
(60) Provisional application No. 63/238,414, filed on Aug. 30, 2021.
(60) Provisional application No. 63/325,502, filed on Mar. 30, 2022.

(51) Int. Cl. C07D 471/08; A61K 31/497; A61K 31/501; A61K 31/506; A61K 31/53; A61K 31/5386; A61P 35/00; A61P 35/02; C07D 401/14; C07D 403/14; C07D 417/14; C07D 471/04; C07D 471/10; C07D 487/04; C07D 487/06; C07D 487/10; C07D 498/08; C07D 519/00

(52) U.S. Cl. CPC C07D 471/08; A61K 31/497; A61K 31/501; A61K 31/506; A61K 31/53; A61K 31/5386; A61P 35/00; A61P 35/02; C07D 401/14; C07D 403/14; C07D 417/14; C07D 471/04; C07D 471/10; C07D 487/04; C07D 487/06; C07D 487/10; C07D 498/08; C07D 519/00

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application claims priority to U.S. Application No. 63/238,409, filed on Aug. 30, 2021; U.S. Application No. 63/238,414, filed on Aug. 30, 2021; and U.S. Application No. 63/325,502, filed on Mar. 30, 2022. The disclosure of each of the foregoing applications is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formula (I), (II), or (III) (e.g., a compound of Formulas (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)) and pharmaceutically acceptable salts, solvates, hydrates, tautomers, or stereoisomers thereof. The present disclosure additionally provides methods of using the compounds of the invention (e.g., compounds of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g), and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g), and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g), and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

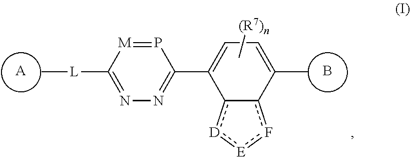

or a pharmaceutically acceptable salt, solvate, hydrate, tautomer, or stereoisomer thereof, wherein A, B, M, P, D, E, F, L, R⁷, n, and subvariables are as defined herein.

In another aspect, the present disclosure provides compounds of Formula (II):

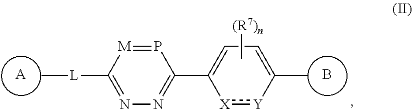

or a pharmaceutically acceptable salt, solvate, hydrate, tautomer, or stereoisomer thereof, wherein A, B, M, P, X, Y, L, R⁷, n, and subvariables are as defined herein.

In another aspect, the present disclosure provides compounds of Formula (III):

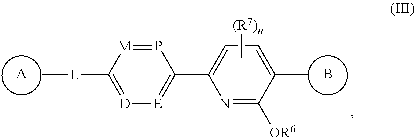

or a pharmaceutically acceptable salt, solvate, hydrate, tautomer, or stereoisomer thereof, wherein A, B, D, E, M, P, L, R⁶, R⁷, n, and subvariables are as defined herein.

In another aspect, the present invention provides pharmaceutical compositions comprising a compound of Formula (I), (II), or (III) (e.g., a compound of Formulas (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)) or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)) or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formula (I), (II), or (III) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formula (I), (II), or (III) alters a target nucleic acid (e.g., DNA or RNA, e.g., a precursor RNA, e.g., a pre-mRNA), target protein, or combination thereof. In some embodiments, the compound of Formula (I), (II), or (III) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), (II), or (III), e.g., in a healthy or diseased cell or tissue). In some embodiments, the presence of a compound of Formula (I), (II), or (III) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), (II), or (III), e.g., in a healthy or diseased cell or tissue).

In another aspect, the present disclosure provides methods for preventing and/or treating a disease, disorder, or condition in a subject by administering a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)) or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)) or a pharmaceutically acceptable salt, solvate, hydrate, tautomer, or stereoisomer thereof in a biological sample or subject. In another aspect, the present disclosure provides methods of altering the isoform of a target protein with a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g))) or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formula (I), (II), or (III) to a biological sample, a cell, or a subject comprises inhibition of cell growth or induction of cell death.

In another aspect, the present disclosure provides compositions for use in preventing and/or treating a disease, disorder, or condition in a subject by administering a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)) or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)) or a pharmaceutically acceptable salt, solvate, hydrate, tautomer, or stereoisomer thereof in a biological sample or subject. In another aspect, the present disclosure provides compositions for use in altering the isoform of a target protein with a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g))) or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formula (I), (II), or (III) to a biological sample, a cell, or a subject comprises inhibition of cell growth or induction of cell death.

In another aspect, the present disclosure features kits comprising a container with a compound of Formula (I), (II), or (III) (e.g., a compound of Formulas (I), (I-a), (I-b), (I-c), (I-d), (I-e), (I-f), (I-g), (I-h), (II-a), (II-b), (II-c), (II-d), (III-a), (III-b), (III-c), (III-d), (III-e), (III-f), or (III-g)), or a pharmaceutically acceptable salt, solvate, hydrate, tautomer, stereoisomer thereof, or a pharmaceutical composition thereof. In certain embodiments, the kits described herein further include instructions for administering the compound of Formula (I), (II), or (III) or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, WO 2021/174170, and WO 2021/174176. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, WO 2021/174170, and WO 2021/174176, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—CI₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6−14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₅), cubanyl (C₅), bicyclo[1.1.1]pentanyl (C₅), bicyclo[2.2.2]octanyl (C₅), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 8 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I), (II), or (III) refers to an attachment point to another moiety or functional group within the compound. Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R—, S—, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and 14C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I), (II), or (III) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formula (I), (II), or (III) refers to an amount sufficient to elicit the desired biological response, i.e., treating the condition. As will be appreciated by those of ordinary skill in this art, the effective amount of a compound of Formula (I), (II), or (III) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I), (II), or (III) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formula (I), (II), or (III)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I), (II), or (III)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### COMPOUNDS

The present disclosure features a compound of Formula (I):

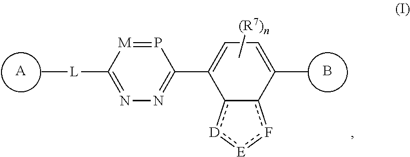

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —S—, —C(O)—, —N(R³)—, —N(R³)C(O)—, or —C(O)N(R³)—, wherein each alkylene and heteroalkylene is optionally substituted with one or more R⁴; M and P are each independently C(R²) or N; D, E, and F are each independently C(R^{5a}) C(R^{5a})(R^{5b}), N, N(R^{5c}), S, or O, wherein at least one of D, E, and F is each independently N or N(R^{5c}), and the bonds between the atoms in the ring comprising D, E, and F may be single bonds or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen C₁-C₆-alkyl, C₂-C₆-alkenyl, C₂-C₆-alkynyl, cycloalkyl, heterocyclyl, halo, cyano, or —OR^{A}; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} and R^{5b} are each independently hydrogen, halo, C₁-C₆ alkyl, or cycloalkyl; or R^{5a} and R^{5b} are taken together to form an oxo group; R^{5c} is hydrogen, C₁-C₆ alkyl, or cycloalkyl; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is independently C₁-C₆-alkyl, C₁-C₆-heteroalkyl, C₁-C₆-haloalkyl, halo, or cycloalkyl; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, or 2; and x is 1, or 2.

The present disclosure further features a compound of Formula (II):

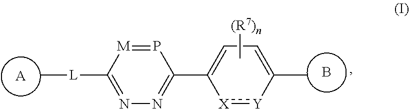

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; M and P are each independently C(R²) or N; X and Y are each independently C(R^{5a}), C(R^{5a})(R^{5b}), N, or N(R^{5c}), wherein the bond between X and Y may be a single or double bond as valency permits, and wherein X and Y may not both be C(R^{5a})(R^{5b}) or C(R^{5a});
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸;
each R² is independently hydrogen, halo, cyano, C₁-C₆-alkyl, C₂-C₆-alkenyl, C₂-C₆-alkynyl, or —OR^{A};
each R³ is independently hydrogen, C₁-C₆-alkyl, or C₁-C₆-haloalkyl;
each of R^{5a} and R^{5b} is C₁-C₆-alkyl, C₂-C₆-alkenyl, C₂-C₆-alkynyl, C₁-C₆-heteroalkyl, C₁-C₆-haloalkyl, cycloalkyl, halo, cyano, oxo, —OR^{A1}, —NR^{B}R^{C}, —C(O)R^{D}, or —C(O)OR^{D};
R^{5a} and R^{5b}, together with the carbon atom to which they are attached, form an oxo group;
each R^{5c} is hydrogen, C₁-C₆-alkyl, C₁-C₆-haloalkyl, cycloalkyl, or C(O)R^{D};
each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹;
R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D};
R^{B} and R^{C} together with the atom to which they are attached form a 3-7-membered heterocyclyl or heteroaryl ring optionally substituted with one or more R¹⁰;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl;
R¹⁰ is C₁-C₆-alkyl or halo;
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A1} is independently C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D};
n is 0, 1, or 2; and
   x is 0, 1, or 2

As generally described herein, each of A or B are independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic ring. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, each of A and B are independently selected from:

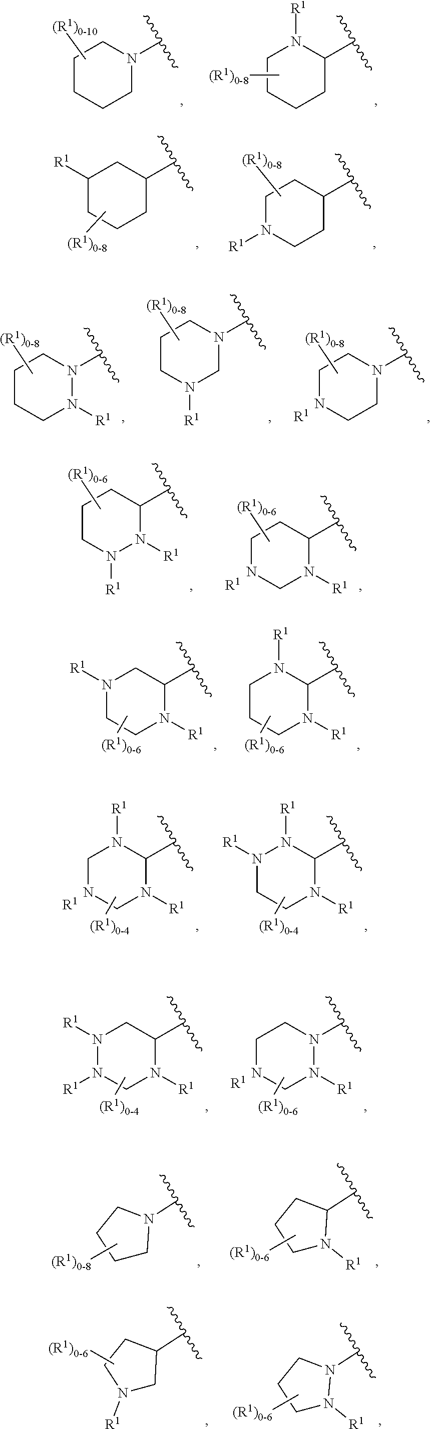

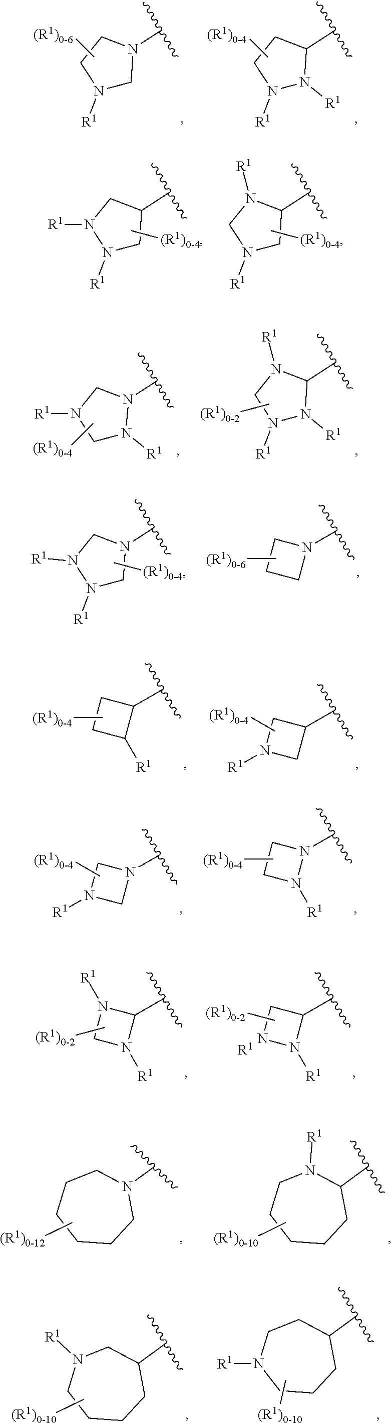

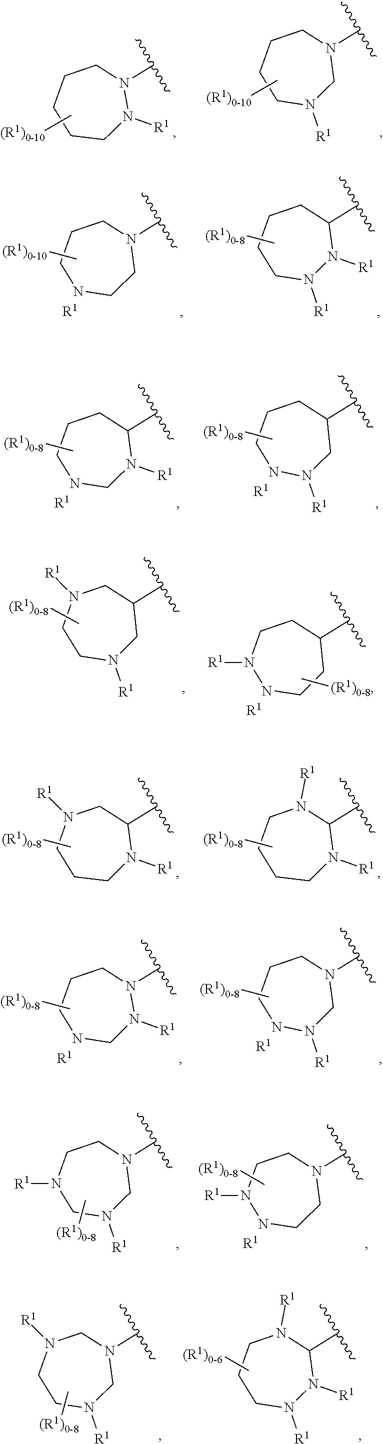

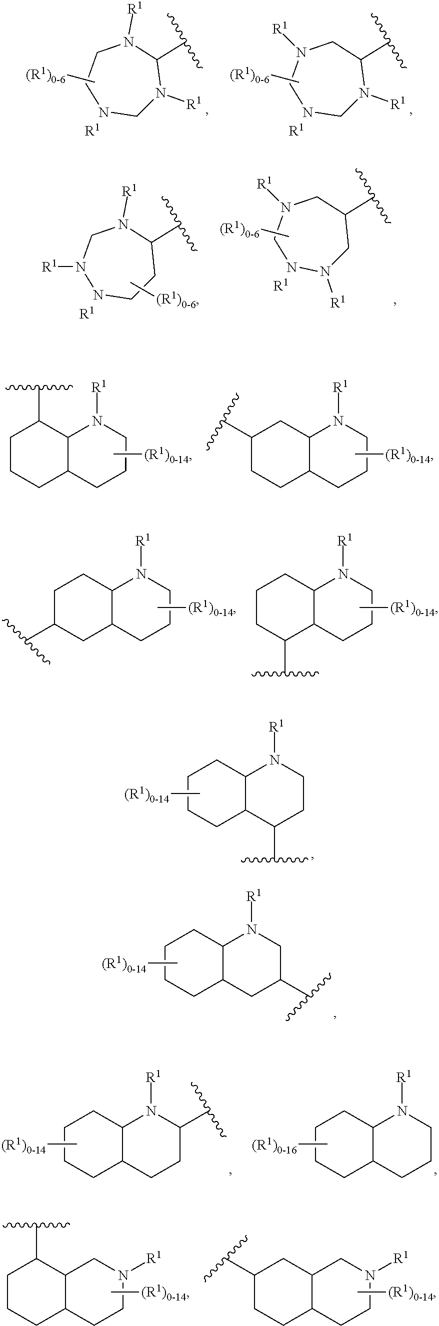

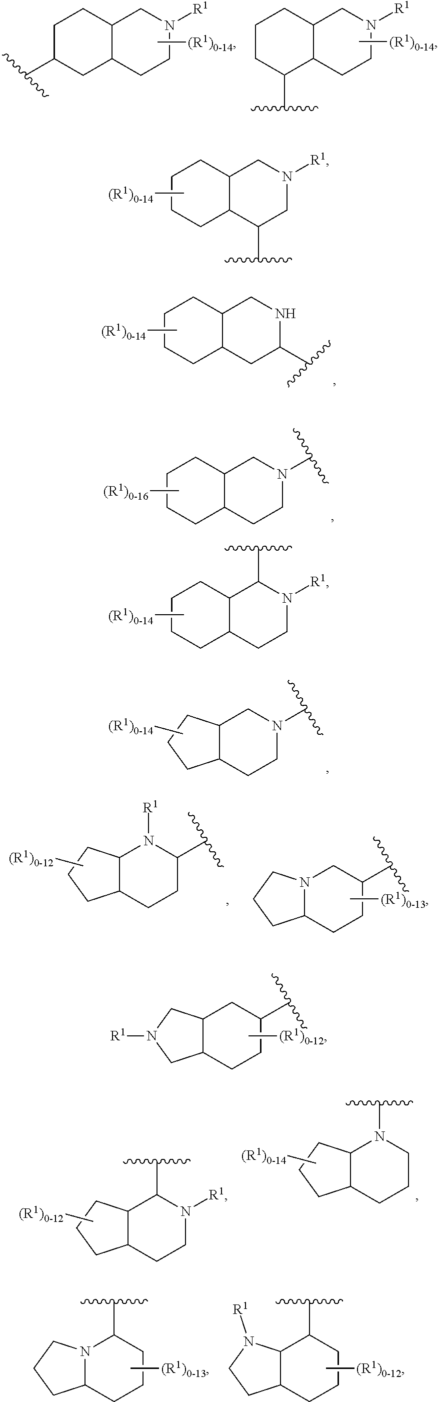

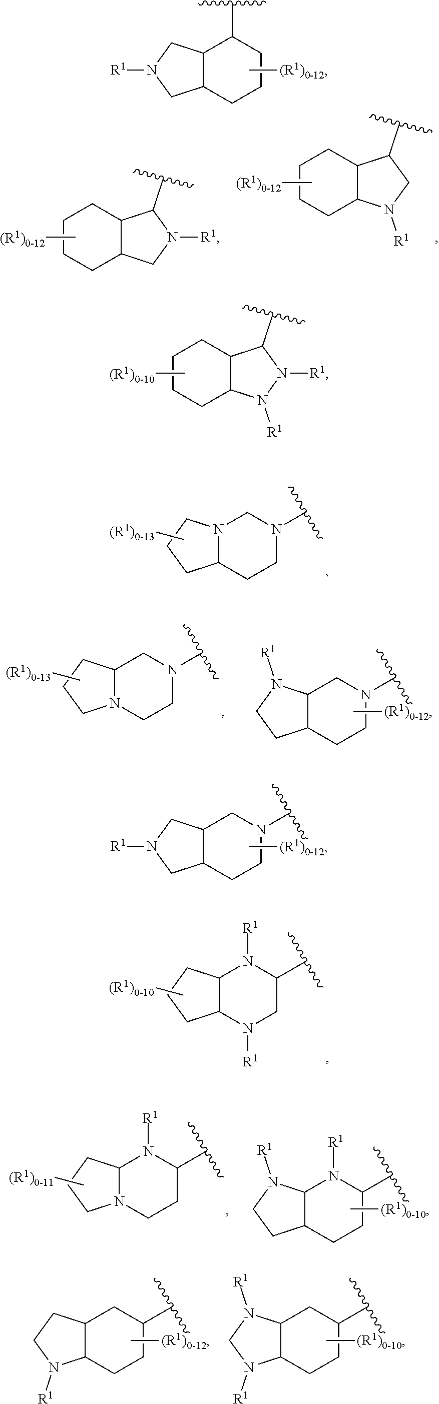

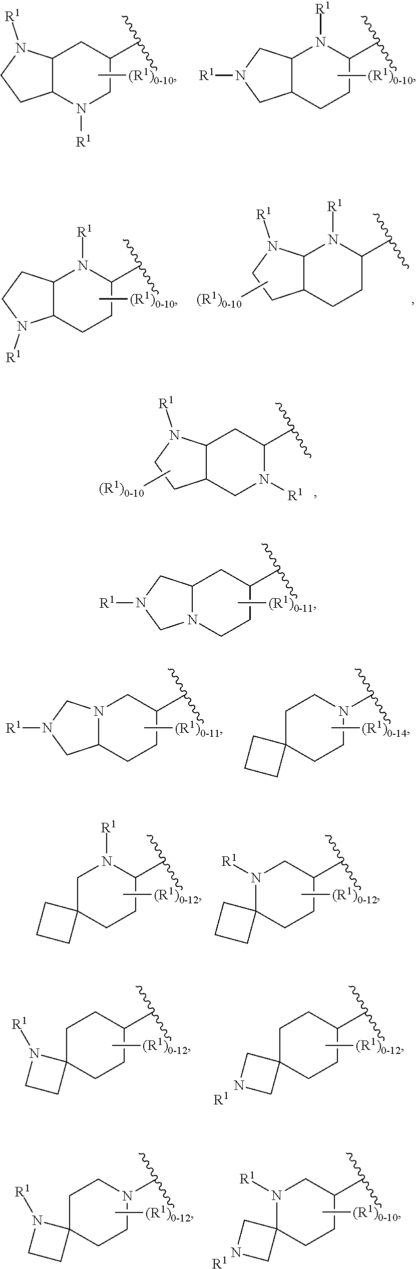

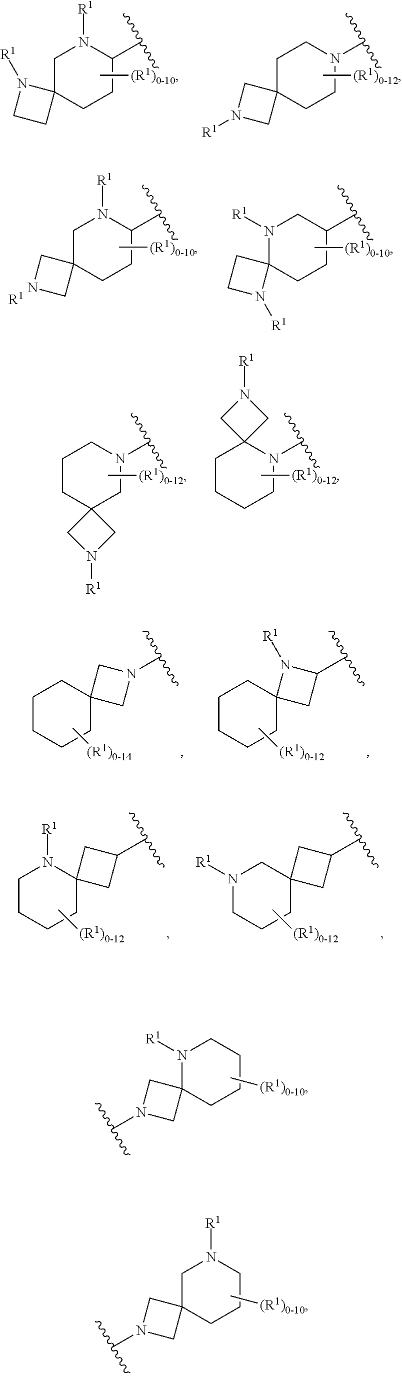

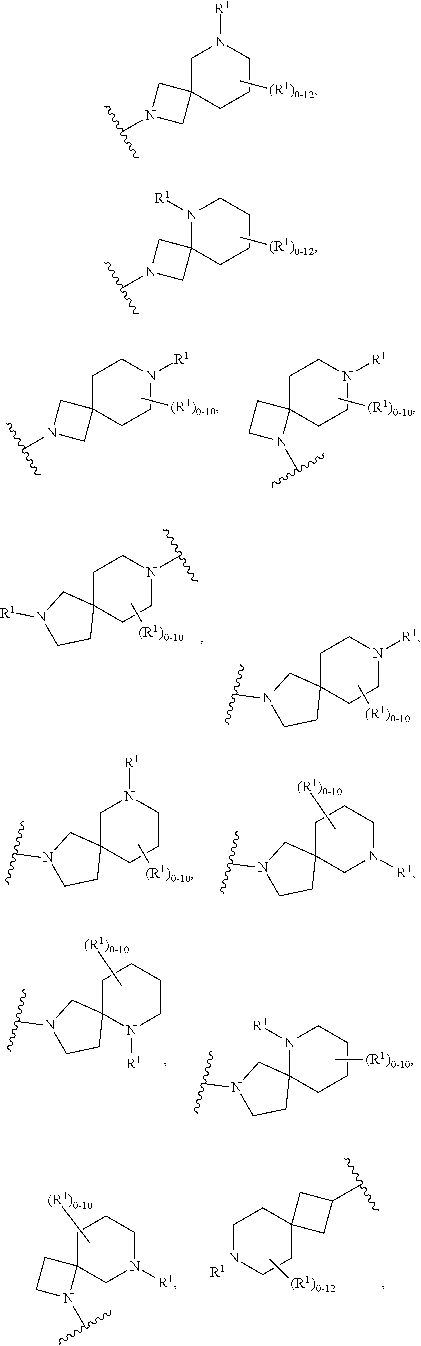

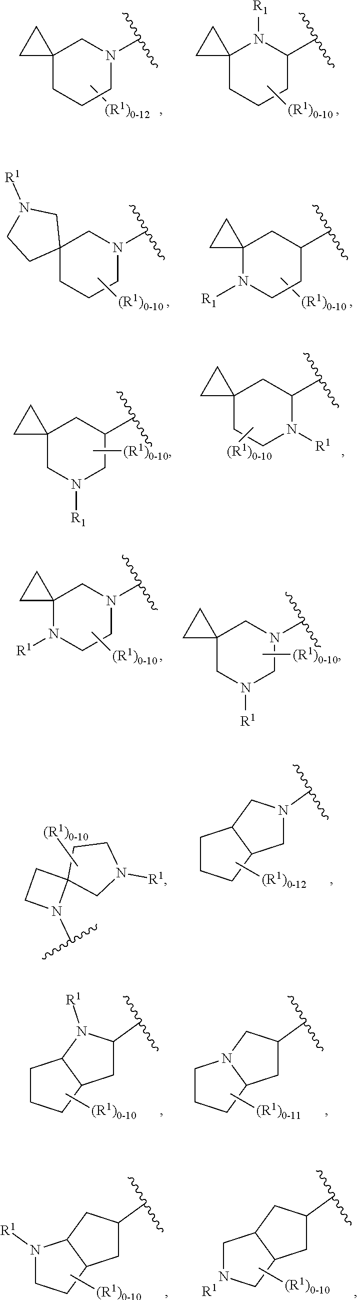

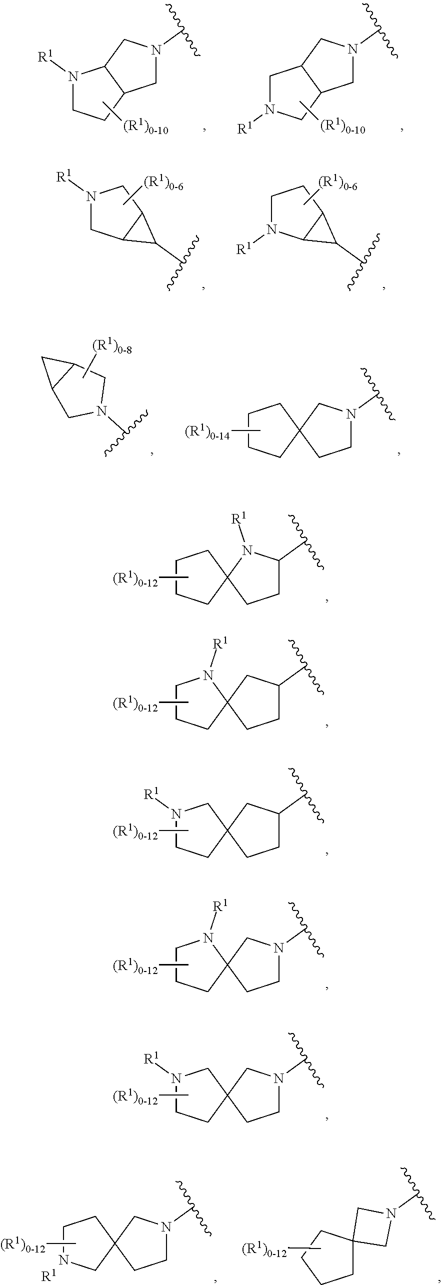

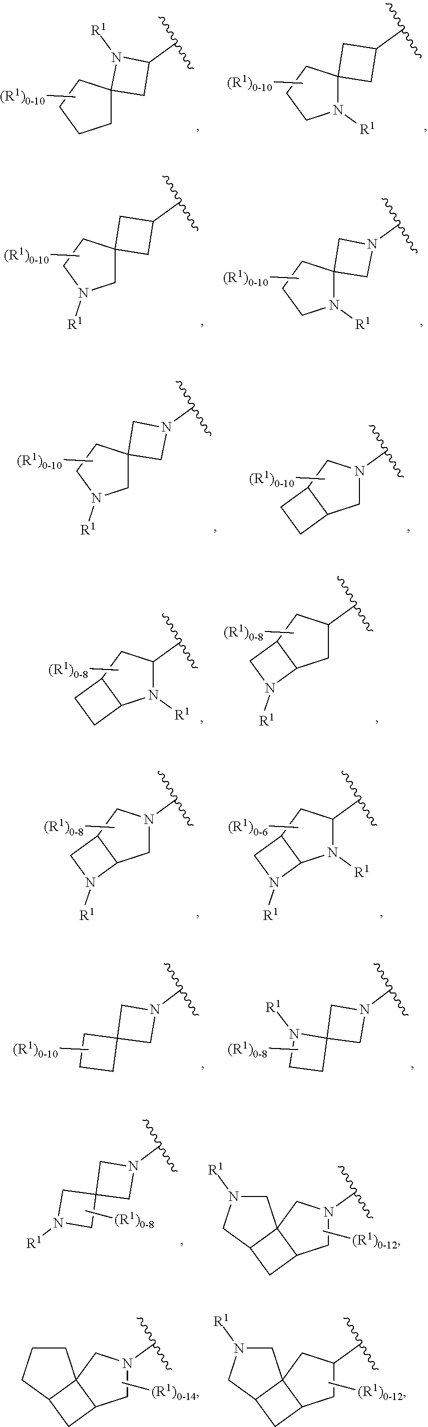

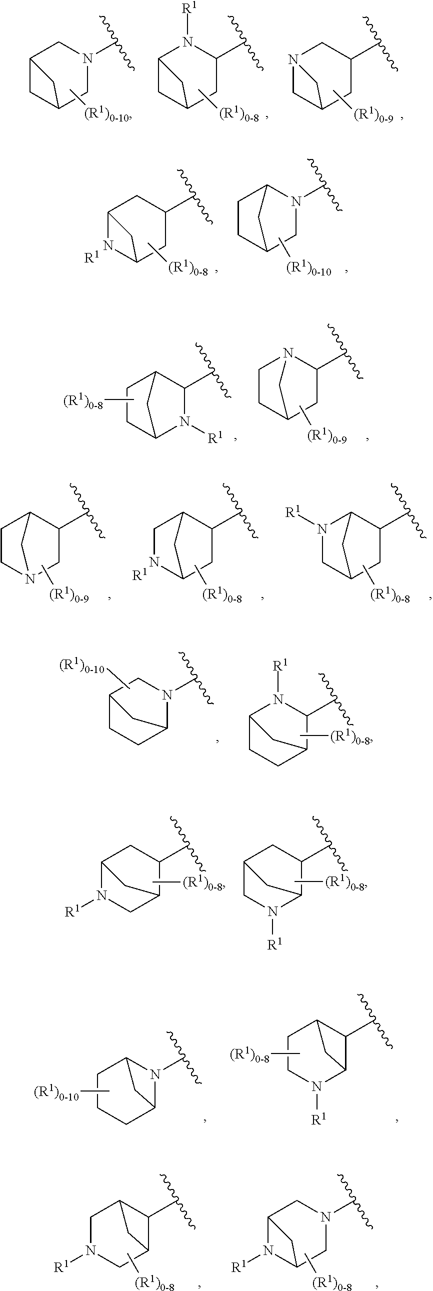

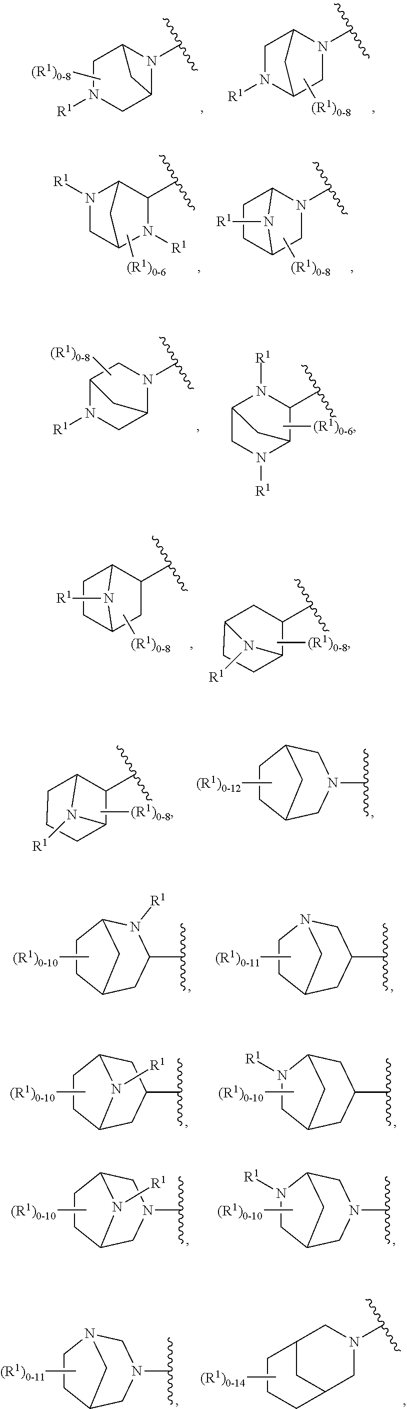

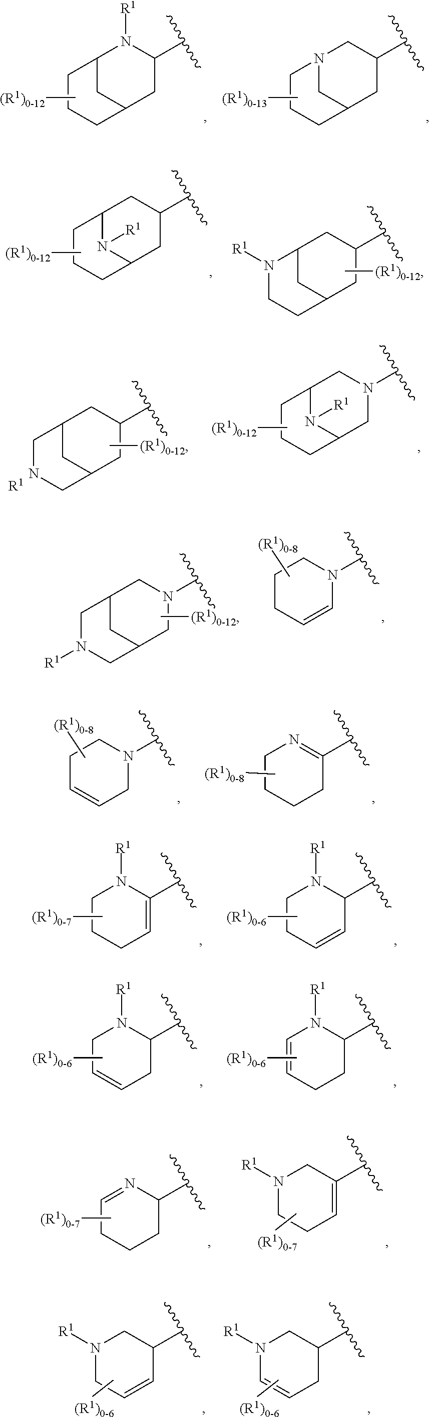

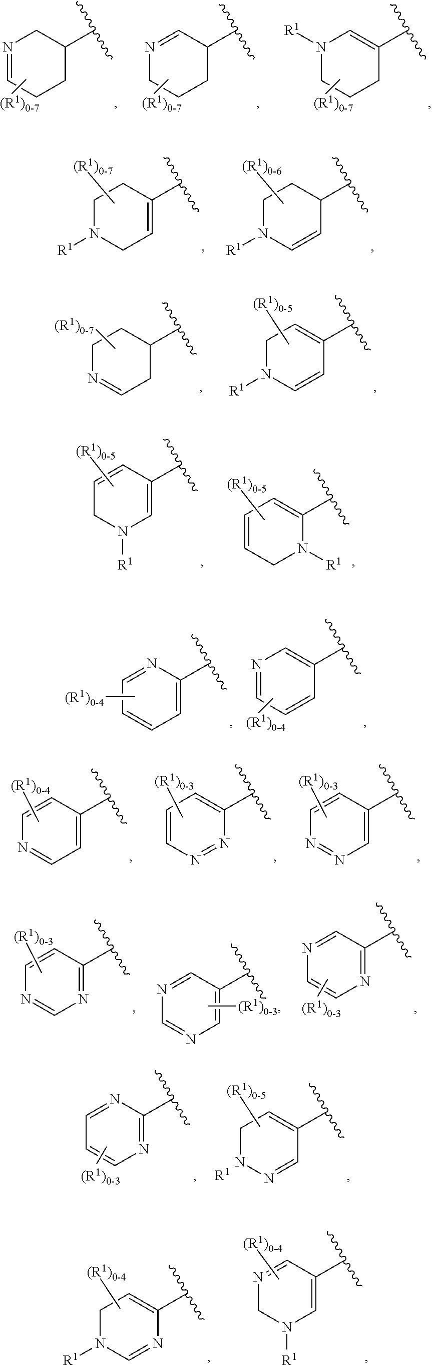

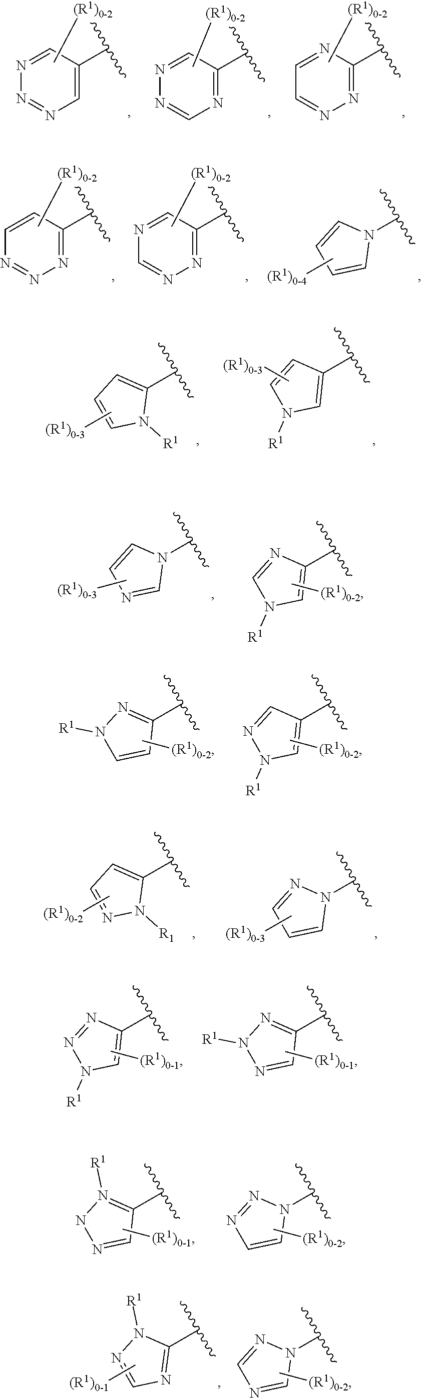

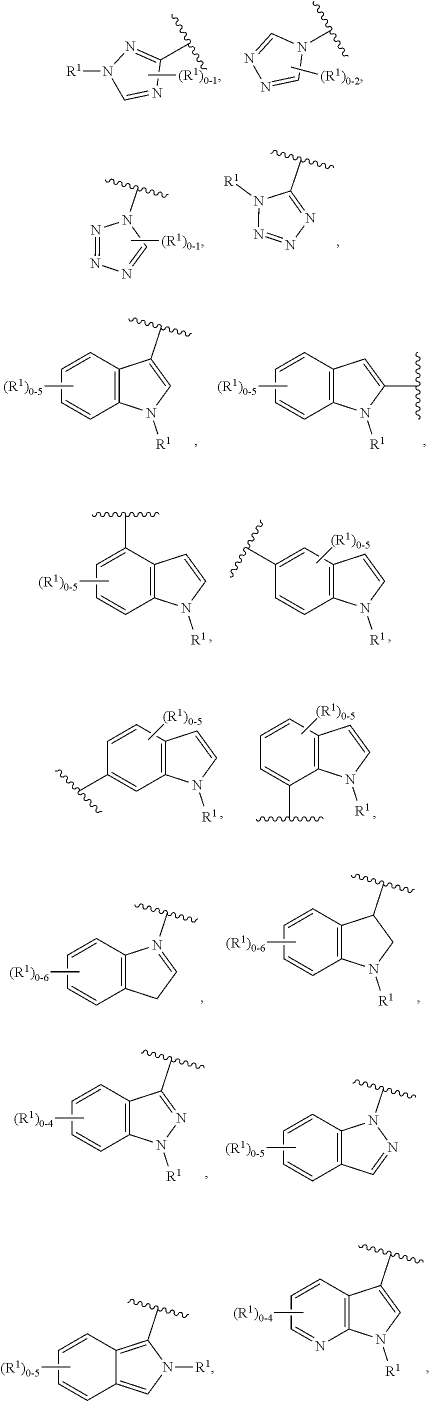

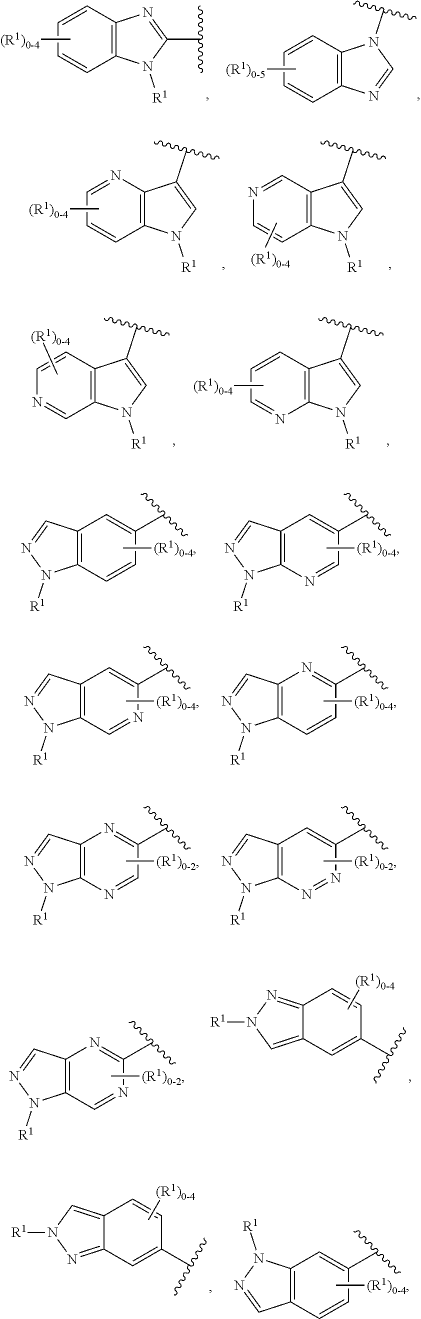

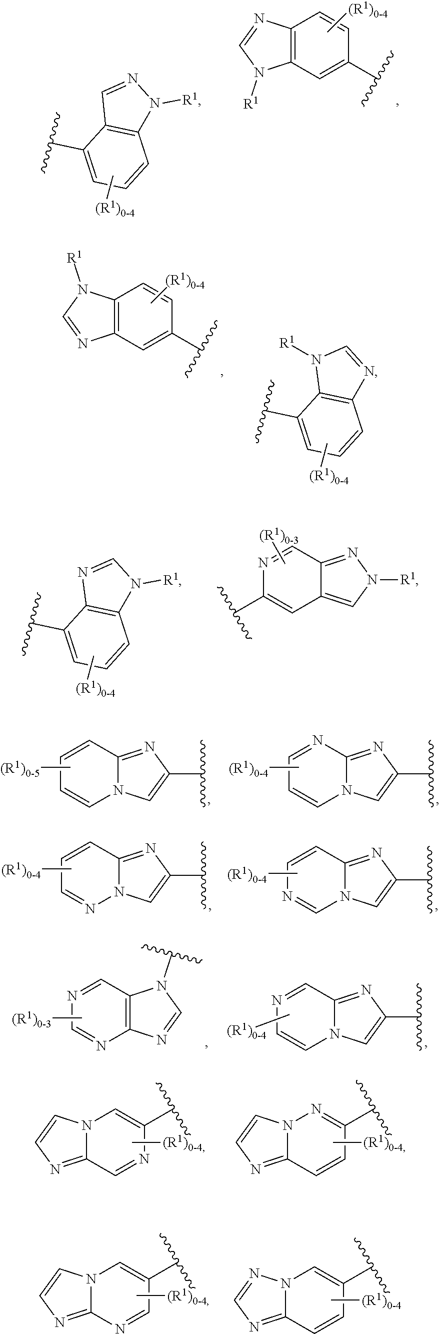

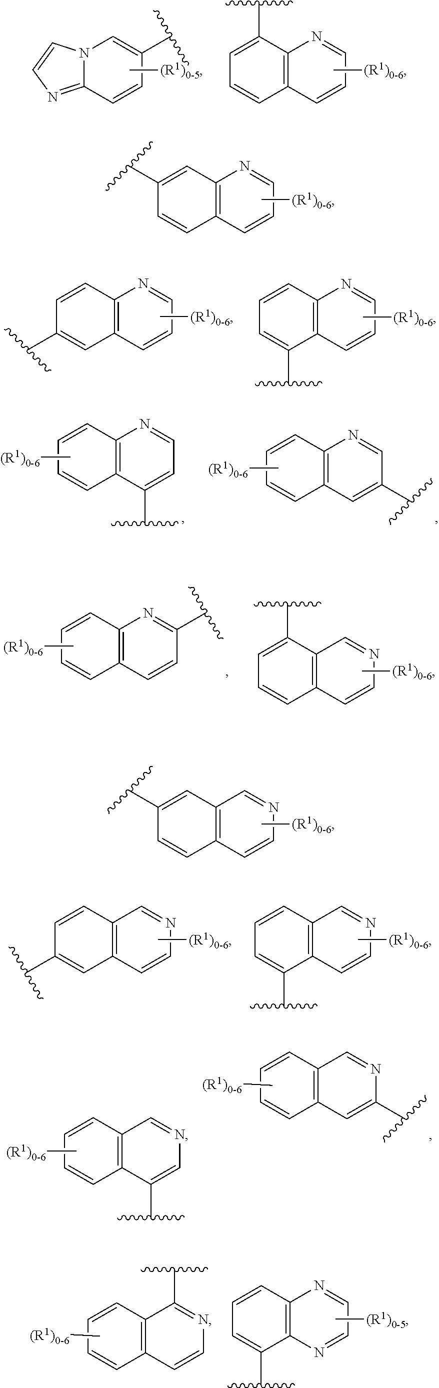

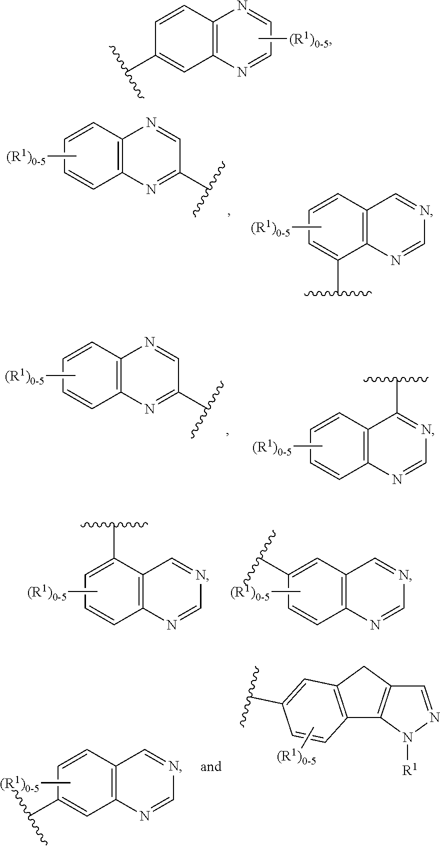

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

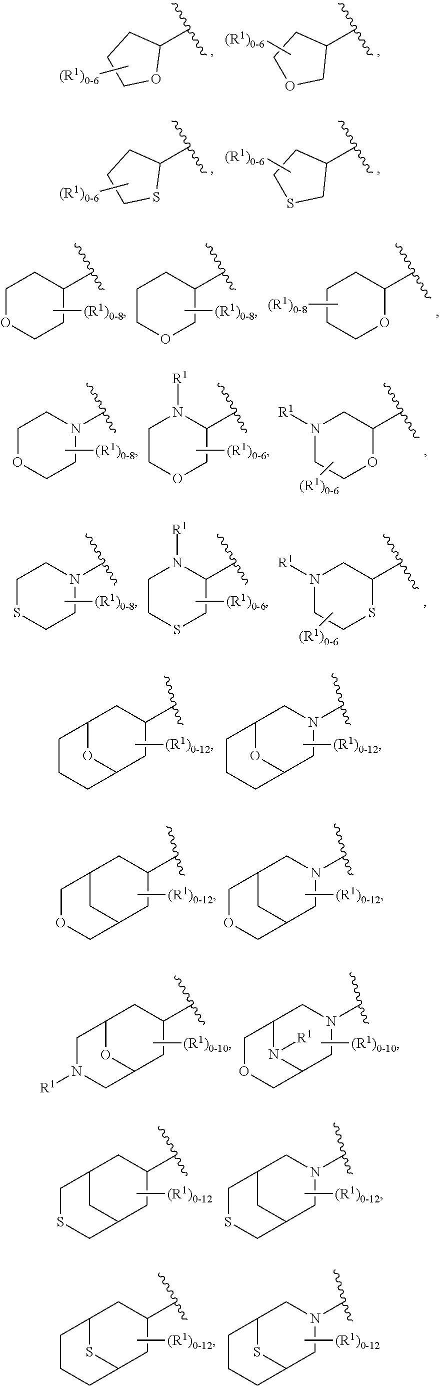

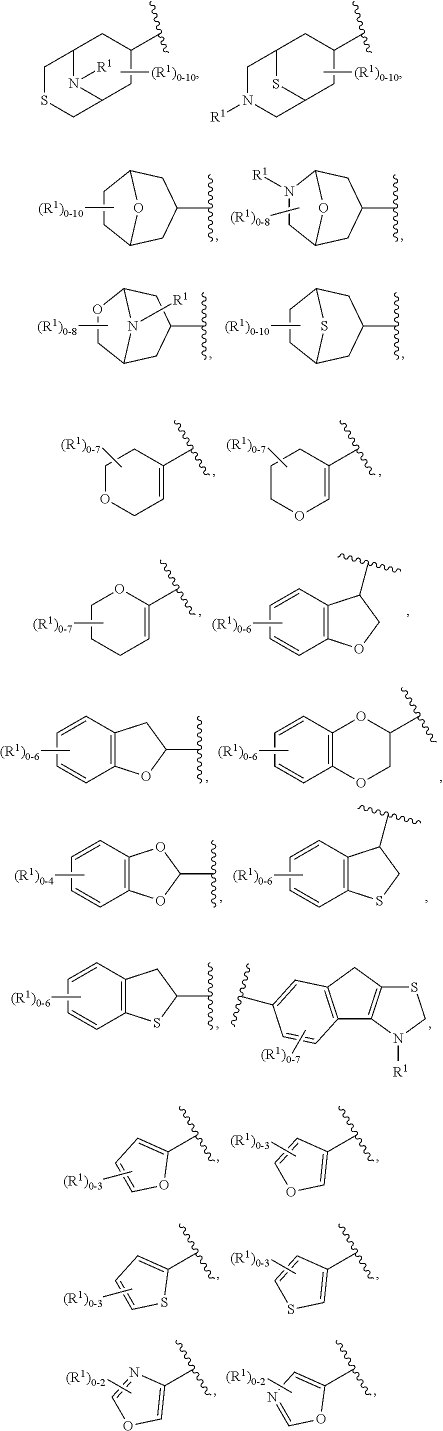

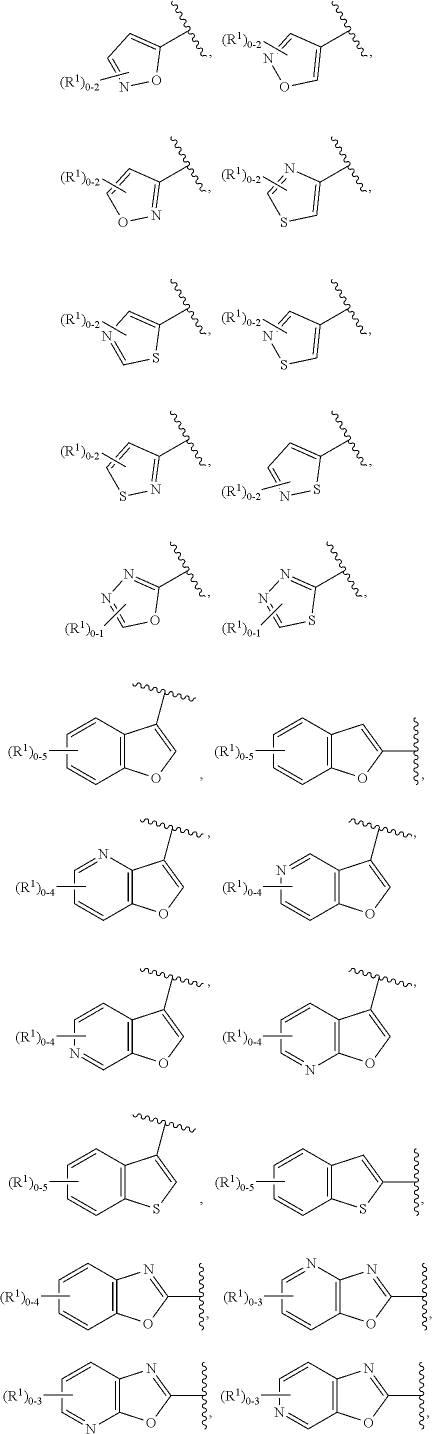

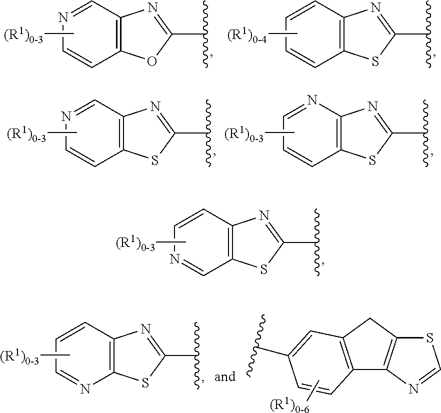

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B is independently heteroaryl or heterocyclyl, each of which is optionally substituted with one or more R¹. In some embodiments, one of A and B is independently a monocyclic heteroaryl or bicyclic heteroaryl, each of which is optionally substituted with one or more R¹. In some embodiments, one of A and B is independently a monocyclic heteroaryl optionally substituted with one or more R¹. In some embodiments, one of A and B is independently a nitrogen-containing heteroaryl optionally substituted with one or more R¹. In some embodiments, one of A and B is independently a 5-6 membered heteroaryl optionally substituted with one or more R¹

In some embodiments, one of A and B is independently selected from

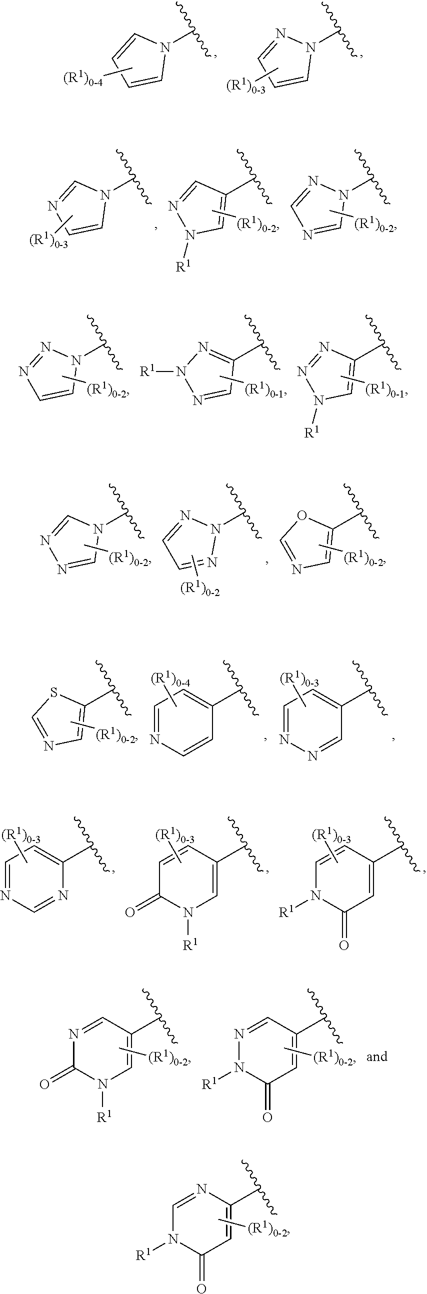

wherein R¹ is as described herein. In some embodiments, one of A and B is independently selected from

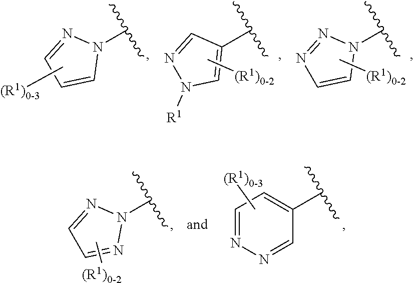

wherein R¹ is as described herein. In some embodiments, one of A and B is independently selected from

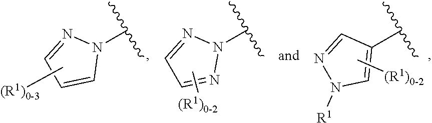

wherein R¹ is as described herein. In some embodiments, B is selected from

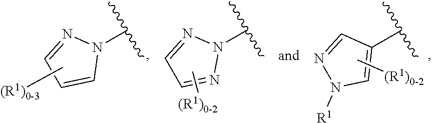

wherein R¹ is as described herein.

In some embodiments, one of A and B is independently selected from

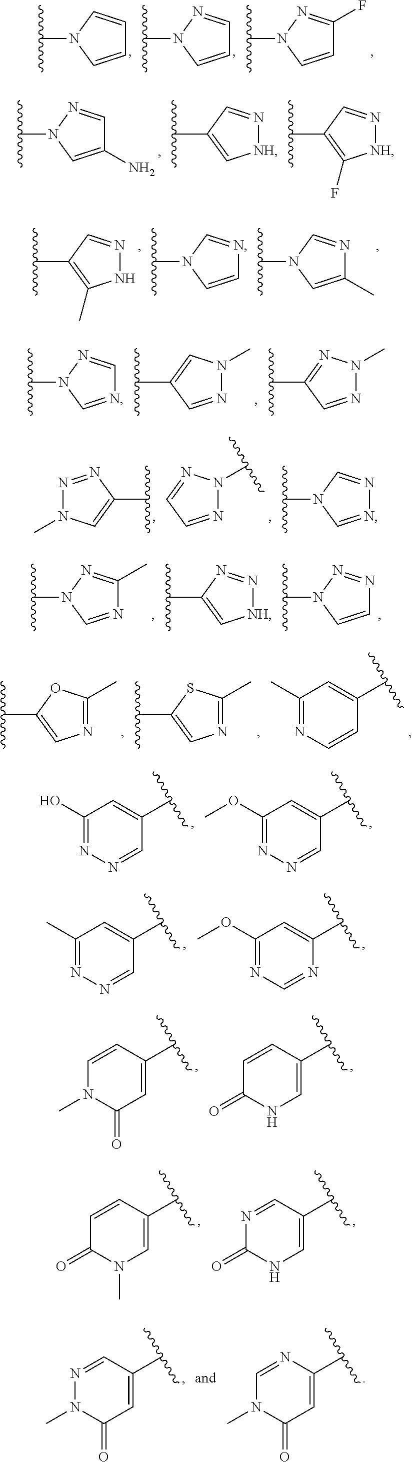

In some embodiments, one of A and B is independently selected from

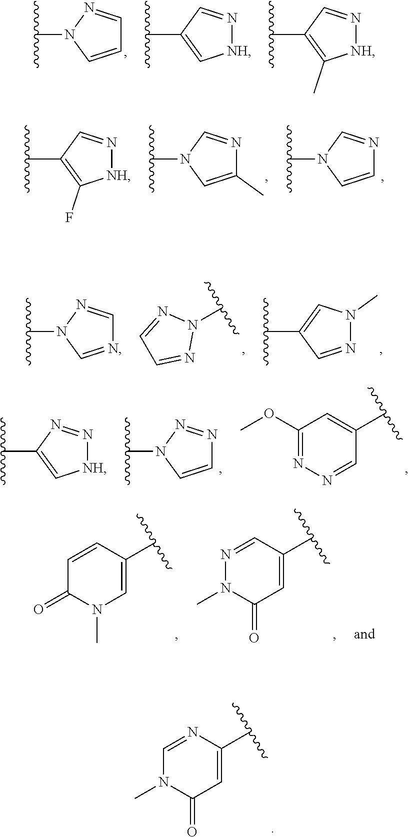

In some embodiments, one of A and B is independently selected from

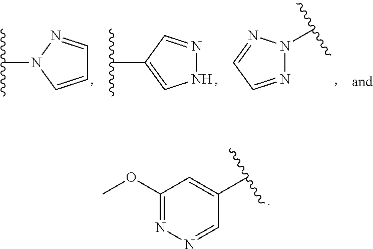

In some embodiments, one of A and B is independently

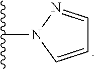 [SMILES: *n1cccn1]

In some embodiments, one of A and B is independently

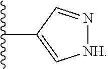 [SMILES: *c1cn[nH]c1]

In some embodiments, one of A and B is independently

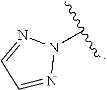 [SMILES: *n1nccn1]

In some embodiments, one of A and B is independently

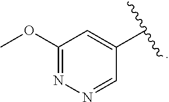 [SMILES: *c1cnnc(OC)c1]

In some embodiments, B is selected from

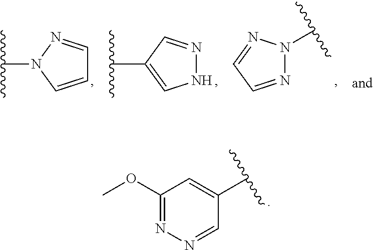

In some embodiments, B is

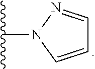 [SMILES: *n1cccn1]

In some embodiments, B is

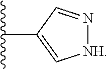 [SMILES: *c1cn[nH]c1]

In some embodiments, B is

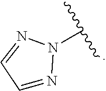 [SMILES: *n1nccn1]

In some embodiments, B is N

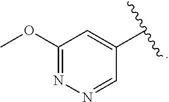 [SMILES: *c1cnnc(OC)c1]

In some embodiments, one of A and B is independently a monocyclic heterocyclyl or bicyclic heterocyclyl, each of which is optionally substituted with one or more R¹. In some embodiments, one of A and B is independently a nitrogen-containing heterocyclyl optionally substituted with one or more R¹. In some embodiments, one of A and B is independently a 4-10 membered heterocyclyl optionally substituted with one or more R¹.

In some embodiments, A is selected one of A and B is independently selected from

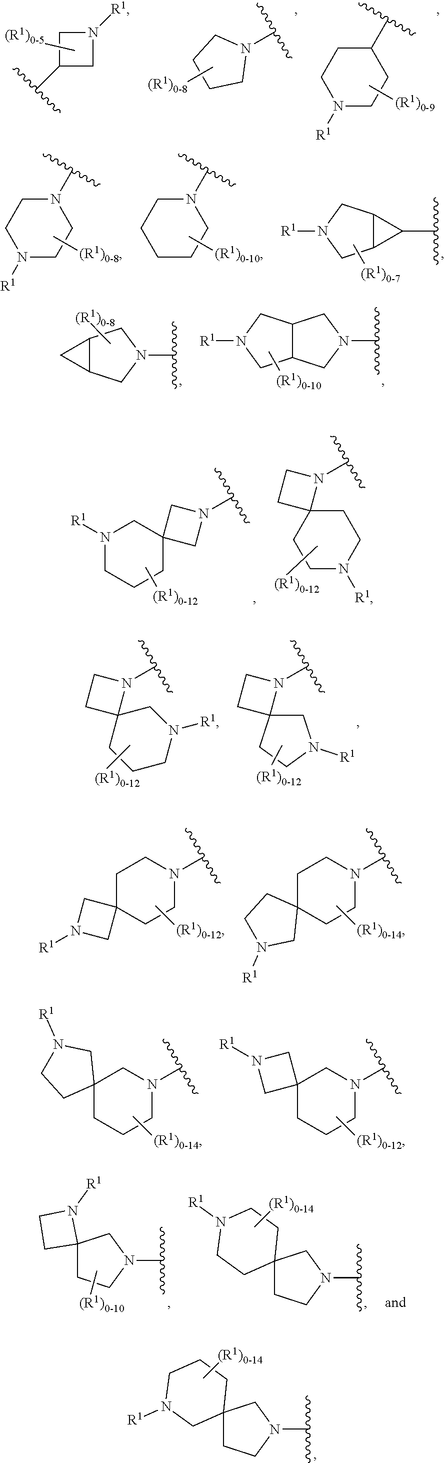

and wherein R¹ is as described herein.

In some embodiments, one of A and B is independently selected from

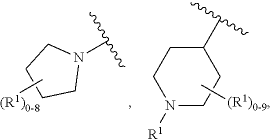

wherein R¹ is as defined herein. In some embodiments, one of A and B is independently selected from

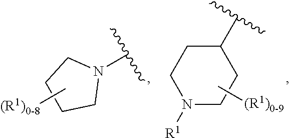

wherein R¹ is as defined herein. In some embodiments, A is

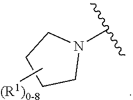

In some embodiments, A is

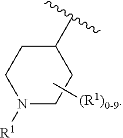

In some embodiments, one of A and B is independently selected from

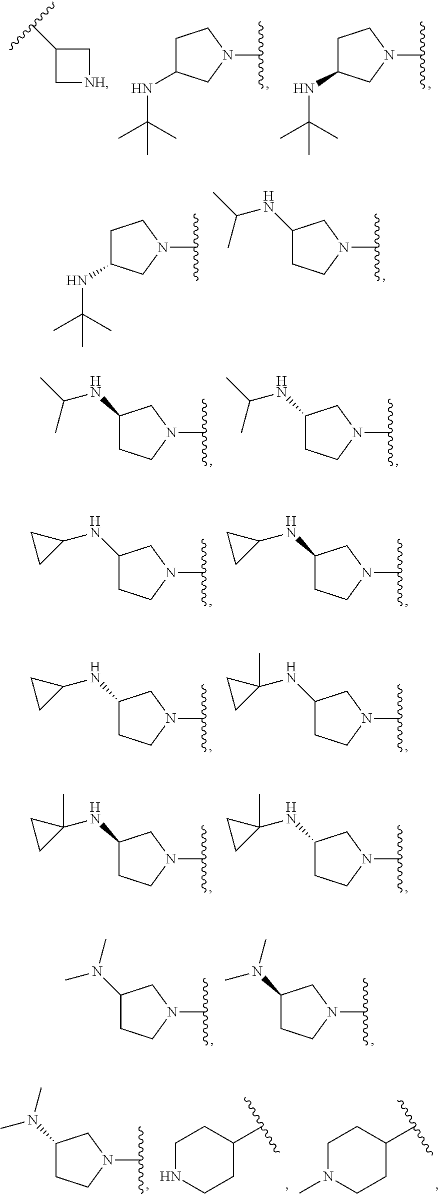

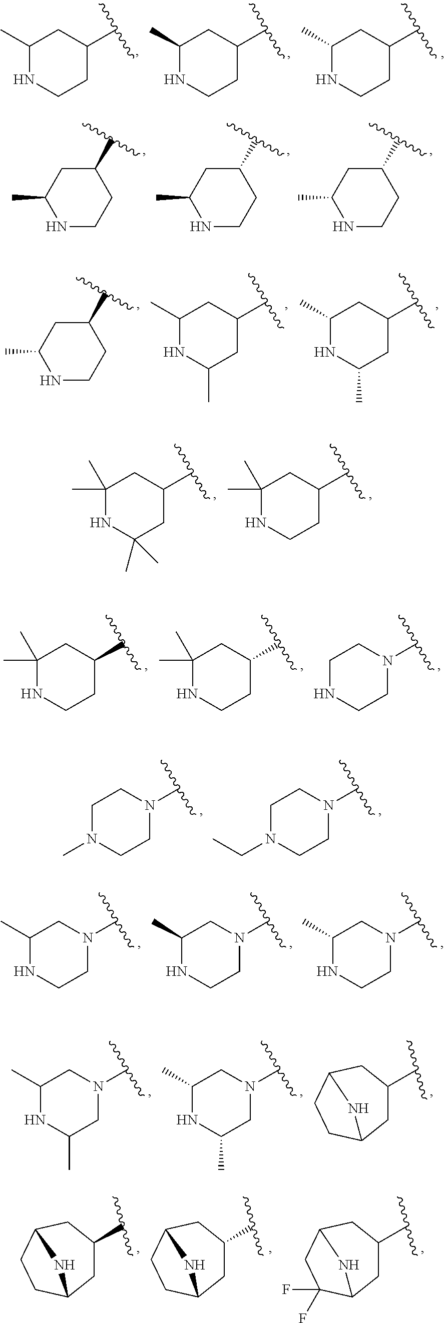

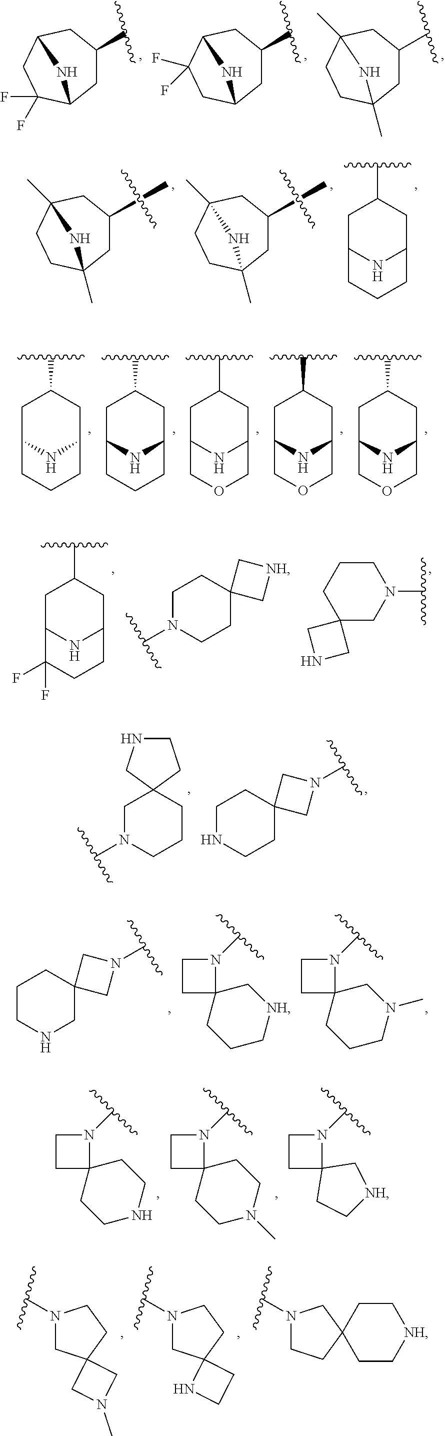

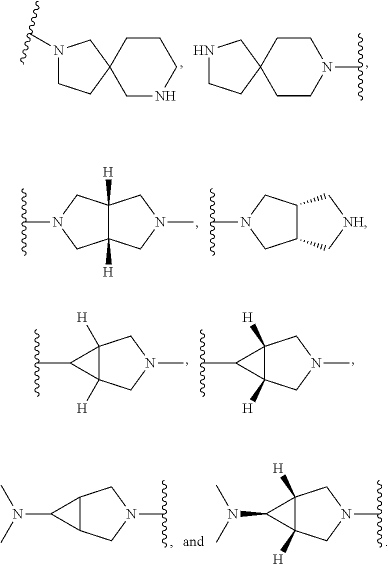

In some embodiments, one of A and B independently selected from

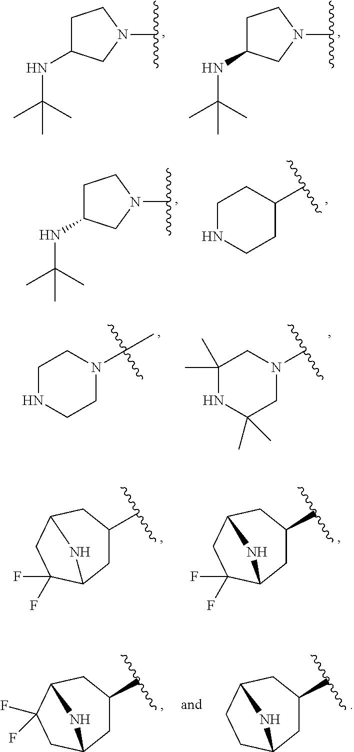

In some embodiments, one of A and B is independently selected from

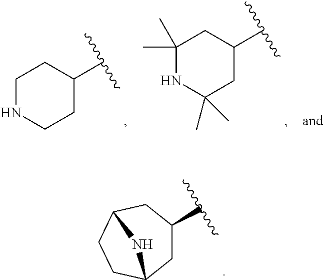

In some embodiments, one of A and B is

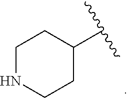 [SMILES: *C1CCNCC1]

In some embodiments, one of A and B is

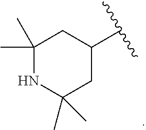 [SMILES: *C1CC(C)(C)NC(C)(C)C1]

In some embodiments, one of A and B is

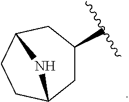 [SMILES: *[C@@H]1C[C@H]2CC[C@@H](C1)N2]

In some embodiments, A is selected from

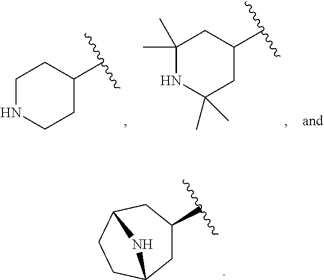

In some embodiments, A is

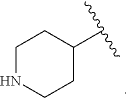 [SMILES: *C1CCNCC1]

In some embodiments, A is

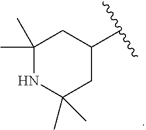 [SMILES: *C1CC(C)(C)NC(C)(C)C1]

In some embodiments, A is

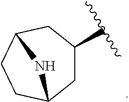 [SMILES: *[C@@H]1C[C@H]2CC[C@@H](C1)N2]

As generally described herein, L may be absent or refer to a C₁-C₆-alkylene, C₁-C₆-heteroalkylene, —O—, —C(O)—, —N(R³)—, —S(O)x, —N(R³)C(O)—, or —C(O)N(R³)— group, wherein each alkylene and heteroalkylene is optionally substituted with one or more R⁴. In some embodiments, L may be absent or refer to a C₁-C₆-alkylene, C₁-C₆-heteroalkylene, —O—, —C(O)—, —N(R³)—, —N(R³)C(O)—, or —C(O)N(R³)— group, wherein each alkylene and heteroalkylene is optionally substituted with one or more R⁴.

In some embodiments, L is absent. In some embodiments, L is C₁-C₆-alkylene (e.g., C₁-alkylene, C₂-alkylene, C₃-alkylene, C₄-alkylene, C₅-alkylene, or C₆-alkylene). In some embodiments, L is unsubstituted C₁-C₆ alkylene. In some embodiments, L is substituted C₁-C₆-alkylene, e.g., C₁-C₆ alkylene substituted with one or more R⁴. In some embodiments, L is C₁-alkylene substituted with one R⁴. In some embodiments, L is —CH₂— (or methylene). In some embodiments, L is —C(O)— (or carbonyl).

In some embodiments, L is absent, C₁-C₆-alkylene, C₁-C₆-heteroalkylene, —N(R³)C(O)—, or —C(O)N(R³)—, wherein each alkylene and heteroalkylene is optionally substituted with one or more R⁴.

In some embodiments, L is C₁-C₆-heteroalkylene (e.g., C₁-heteroalkylene, C₂-heteroalkylene, C₃-heteroalkylene, C₄-heteroalkylene, C₅-heteroalkylene, or C₆-heteroalkylene). In some embodiments, L is unsubstituted C₁-C₆ heteroalkylene. In some embodiments, L is C₁-C₆-heteroalkylene substituted with one or more R⁴. In some embodiments, the heteroalkylene comprises 1 or more heteroatoms. In some embodiments, the heteroalkylene comprises one or more of oxygen, sulfur, nitrogen, boron, silicon, or phosphorus. In some embodiments, L is —N(R³)C(O)—. In some embodiments, L is —C(O)N(R³)—.

In some embodiments, L is oxygen. In some embodiments, L is nitrogen, which may be substituted with R³. In some embodiments, L is nitrogen substituted with one R³. In some embodiments, L is —N(R³)—. In some embodiments, L is —N(CH₃)—. In some embodiments, L is —NH—. In some embodiments, L is —O—.

In some embodiments, L is —S(O)ₓ—. In some embodiments, x is 0, 1, or 2. In some embodiments, L is —S— or —S(O)₂—. In some embodiments, L is —S—.

As generally described herein, each of M and P independently refer to C(R²) or N. In some embodiments, each of M and P is independently C(R²) or N. In some embodiments, M and P are each independently C(R²), e.g., CH. In some embodiments, one of M and P is C(R²), and the other of M and P is N. In some embodiments, M is C(R²). In some embodiments, M is N. In some embodiments, P is C(R²). In some embodiments, P is N. In some embodiments, M is C(R²) (e.g., CH) and P is N. In some embodiments, M is N and P is C(R²) (e.g., CH).

In some embodiments,

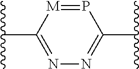

is selected from

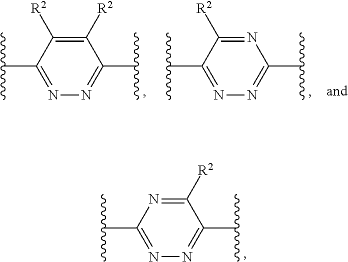

wherein R² is as defined above. In some embodiments, R² is hydrogen.

In some embodiments, one of D, E, and F is N(R^{5c}), and the others of D, E, and F are each independently C(R^{5a}). In some embodiments, two of D, E, and F are each independently N(R^{5c}) or N, and the other of D, E, and F is C(R^{5a}). In some embodiments, three of D, E, and F are each independently N(R^{5c}) or N. In some embodiments, D is N(R^{5c}), and each of E and F are independently C(R^{5a}). In some embodiments, D is N(R^{5c}), E is C(R^{5a}), and F is N. In some embodiments, D is N(R^{5c}), and each of E and F are independently N. In some embodiments, one of D, E, and F is NH, and the others of D, E, and F are independently CH or N.

In some embodiments, one of D, E, and F is N(R^{5c}) (e.g., NH), and the others of D, E, and F are independently O or C(R^{5a})(R^{5b}) (e.g., C(O)). In some embodiments, D is N(R^{5c}) (e.g., CH), E, is C(R^{5a})(R^{5b}) (e.g., C(O)), and F is O. In some embodiments, D is O, E, is C(R^{5a})(R^{5b}) (e.g., C(O)), and F is N(R^{5c}) (e.g., NH). In some embodiments, D is NH, E, is C(O), and F is O. In some embodiments, D is O, E, is C(O), and F is NH.

In some embodiments, one of D, E, and F is S, and the others of D, E, and F, are each independently N or C(R^{5a}) (e.g. CH). In some embodiments, one of D, E, and F is S, and the others of D, E, and F, are each independently C(R^{5a}) (e.g., CH). In some embodiments, D is S, and E and F are each independently N or C(R^{5a}) (e.g., CH). In some embodiments, D is S, E is C(R^{5a}) (e.g., CH), and F is N. In some embodiments, D is S, and E and F are each independently C(R^{5a}) (e.g., CH). In some embodiments, D is S, E is CH, and F is N. In some embodiments, D is S, and E and F are each independently CH.

In some embodiments, one of D, E, and F is N(R^{5f}), and the others of D, E, and F are each independently C(R^{5a})(R^{5a}). In some embodiments, two of D, E, and F are each independently N(R^{5c}), and the other of D, E, and F is C(R^{5a})(R^{5b}). In some embodiments, D is N(R^{5c}), and each of E and F are independently C(R^{5a})(R^{5b}). In some embodiments, E is N(R^{5c}) and each of D and F are independently C(R^{5a})(R^{5b}). In some embodiments, F is N(R^{5c}), and each of D and E are independently C(R^{5a})(R^{5b}). In some embodiments, D is NH, E is C(O), and F is CH₂. In some embodiments, D is CH₂, E is NH, and F is C(O). In some embodiments, D is NH E is CH, and F is C(O).

In some embodiments,

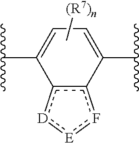

is selected from

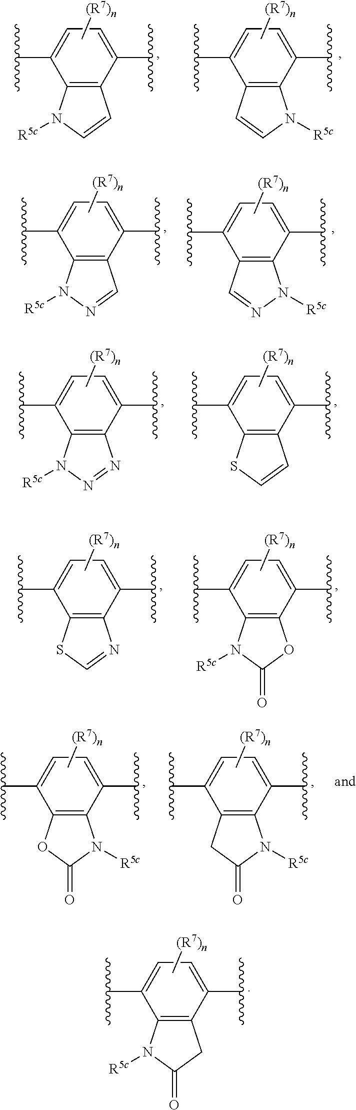

In some embodiments,

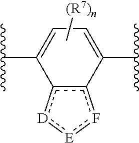

is selected from

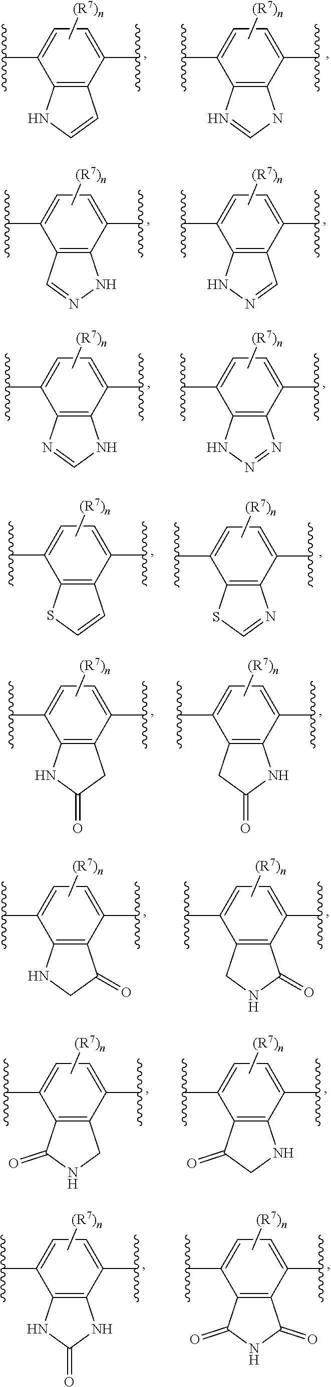

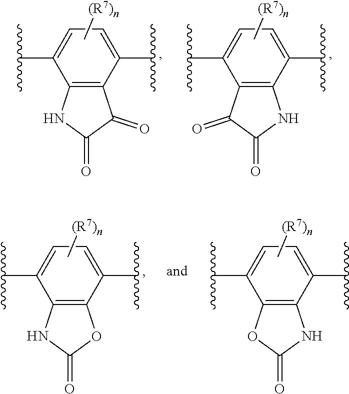

O or a tautomer thereof, wherein R⁷ and n are as defined above. In some embodiments,

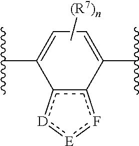

is selected from

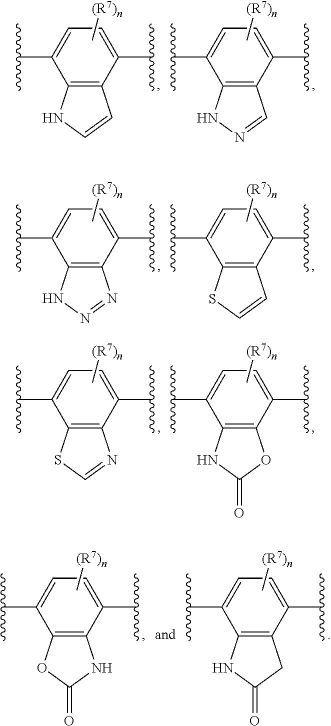

In some embodiments,

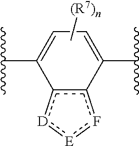

is selected from

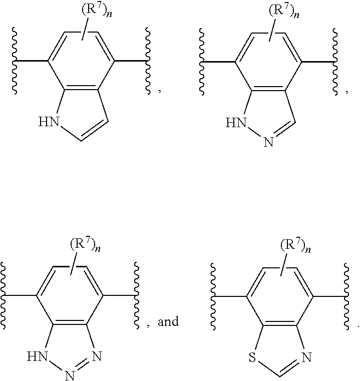

In some embodiments,

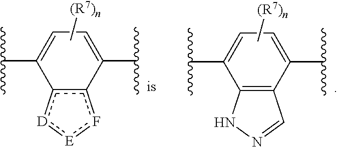

In some embodiments, R¹ is hydrogen. In some embodiments, ^{R} is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁸. In some embodiments, R¹ is C₂-C₆-alkenyl substituted with one or more R⁸. In some embodiments, R¹ is C₂-C₆-alkynyl substituted with one or more R⁸. In some embodiments, R¹ is C₁-C₆-heteroalkyl substituted with one or more R⁸. In some embodiments, R¹ is C₁-C₆-haloalkyl substituted with one or more R⁸. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁸. In some embodiments, R¹ is heterocyclyl substituted with one or more R⁸. In some embodiments, R¹ is aryl substituted with one or more R⁸. In some embodiments, R¹ is C₁-C₆ alkylene-aryl substituted with one or more R⁸. In some embodiments, R¹ is C₁-C₆ alkenylene-aryl substituted with one or more R⁸. In some embodiments, R¹ is C₁-C₆ alkylene-heteroaryl substituted with one or more R⁸. In some embodiments, R¹ is heteroaryl substituted with one or more R⁸.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is —C(O)NR^{B}R^{C}. In some embodiments, R¹ is —C(O)R^{D}. In some embodiments, R¹ is —C(O)OR^{D}. In some embodiments, R¹ is-SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁸.

In some embodiments, R² is hydrogen. In some embodiments, R² is C₁-C₆ alkyl. In some embodiments, R² is C₂-C₆-alkenyl. In some embodiments, R² is C₂-C₆-alkynyl. In some embodiments, R² is C₁-alkyl (e.g., methyl). In some embodiments, R² is methyl. In some embodiments, R² is —OR^{A}. In some embodiments, R² is halo (e.g., fluoro, chloro, bromo, or iodo). In some embodiments, R² is fluoro. In some embodiments, R² is cyano.

In some embodiments, R³ is hydrogen. In some embodiments, R³ is C₁-C₆ alkyl. In some embodiments, R³ is C₁-C₆ haloalkyl. In some embodiments, R³ is C₁-alkyl (e.g., methyl). In some embodiments, R³ is methyl. In some embodiments, R³ is modified with one or more R¹². In some embodiments, R¹² is deuterium. In some embodiments, R³ is CD₃.

In some embodiments, R⁴ is C₁-C₆-alkyl. In some embodiments, R⁴ is C₁-C₆-heteroalkyl. In some embodiments, R⁴ is C₁-C₆-haloalkyl. In some embodiments, R⁴ is cycloalkyl. In some embodiments, R⁴ is halo (e.g., fluoro, chloro, bromo, or iodo). In some embodiments, R⁴ is cyano. In some embodiments, R⁴ is oxo. In some embodiments, R⁴ is —OR^{A}. In some embodiments, R⁴ is —NR^{B}R^{C}. In some embodiments, R⁴ is —C(O)R^{D} or —C(O)OR^{D}.

In some embodiments, R^{5a} and R^{5b} are each independently hydrogen or C₁-C₆-alkyl. In some embodiments, R^{5a}, is hydrogen. In some embodiments, R^{5b} is hydrogen. In some embodiments, R^{5a} and R^{5b} are taken together to form an oxo group. In some embodiments, R^{5c}, is hydrogen. In some embodiments, R^{5c}, is C₁-C₆-alkyl. In some embodiments, R⁵, is C₁-C₆-haloalkyl (e.g., —CF₃ or —CHF₂). In some embodiments, R^{5c}, is —CF₃. In some embodiments, R^{5c}, is —CHF₂. In some embodiments, R^{5c} is —C(O)R^{D} (e.g., —C(O)CH₃). In some embodiments, R^{5c}, is —C(O)CH₃).

In some embodiments, R⁷ is C₁-C₆-alkyl. In some embodiments, R⁷ is C₂-C₆-alkenyl. In some embodiments, R⁷ is C₂-C₆-alkynyl. In some embodiments, R⁷ is C₁-C₆-heteroalkyl. In some embodiments, R⁷ is C₁-C₆-haloalkyl. In some embodiments, R⁷ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R⁷ is C₁-C₆-alkyl substituted with one or more R⁹. In some embodiments, R⁷ is C₂-C₆-alkenyl substituted with one or more R⁹. In some embodiments, R⁷ is C₂-C₆-alkynyl substituted with one or more R⁹. In some embodiments, R⁷ is C₁-C₆-heteroalkyl substituted with one or more R⁹. In some embodiments, R⁷ is C₁-C₆-haloalkyl substituted with one or more R⁹. In some embodiments, R⁷ is halo, e.g., fluoro, chloro, bromo, or iodo. In some embodiments, R⁷ is fluoro. In some embodiments, R⁷ is cyano. In some embodiments, R⁷ is oxo. In some embodiments, R⁷ is NR^{B}C(O)R^{D}. In some embodiments, R⁷ is —C(O)NR^{B}R^{C}. In some embodiments, R⁷ is —C(O)R^{D}. In some embodiments, R⁷ is —SR^{E}.

In some embodiments, two R⁷ groups, together with the atoms to which they are attached (e.g., X or Y) form a 4-7-membered cycloalkyl. In some embodiments, two R⁷ groups, together with the atoms to which they are attached (e.g., X or Y) form a 4-7-membered heterocyclyl. In some embodiments, two R⁷ groups, together with the atoms to which they are attached (e.g., X or Y) form a 5- or 6-membered aryl. In some embodiments, two R⁷ groups, together with the atoms to which they are attached (e.g., X or Y) form a 5- or 6-membered heteroaryl. In some embodiments, two R⁷ groups, together with the atoms to which they are attached (e.g., X or Y), form a 5-membered heterocyclyl. In some embodiments, two R⁷ groups, together with the atoms to which they are attached (e.g., X or Y), form a 5-membered heteroaryl. The cycloalkyl, heterocyclyl, aryl, or heteroaryl may be substituted with one or more R⁹.

In some embodiments, R⁸ is C₁-C₆-alkyl. In some embodiments, R⁸ is C₂-C₆-alkenyl. In some embodiments, R⁸ is C₂-C₆-alkynyl. In some embodiments, R⁸ is C₁-C₆-heteroalkyl. In some embodiments, R⁸ is C₁-C₆-haloalkyl. In some embodiments, R⁸ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁸ is C₁-C₆-alkyl substituted with one or more R¹¹. In some embodiments, R⁸ is C₂-C₆-alkenyl substituted with one or more R¹¹. In some embodiments, R⁸ is C₂-C₆-alkynyl substituted with one or more R¹¹. In some embodiments, R⁸ is C₁-C₆-haloalkyl substituted with one or more R¹¹. In some embodiments, R⁸ is C₁-C₆-heteroalkyl substituted with one or more R¹¹.

In some embodiments, R⁸ is cycloalkyl. In some embodiments, R⁸ is heterocyclyl. In some embodiments, R⁸ is aryl. In some embodiments, R⁸ is heteroaryl. In some embodiments, R⁸ is unsubstituted cycloalkyl, unsubstituted heterocyclyl, unsubstituted aryl, or unsubstituted heteroaryl. In some embodiments, R⁸ is cycloalkyl substituted with one or more R¹¹. In some embodiments, R⁸ is heterocyclyl substituted with one or more R¹¹. In some embodiments, R⁸ is aryl substituted with one or more R¹¹. In some embodiments, R⁸ is heteroaryl substituted with one or more R¹¹.

In some embodiments, R⁸ is halo (e.g., fluoro, chloro, bromo, or iodo). In some embodiments, R⁸ is cyano. In some embodiments, R⁸ is oxo. In some embodiments, R⁸ is —OR^{A}. In some embodiments, R⁸ is —NR^{B}R^{C}. In some embodiments, R⁸ is —NR^{B}C(O)R^{D}. In some embodiments, R⁸ is —NO₂. In some embodiments, R⁸ is —C(O)NR^{B}R^{C}. In some embodiments, R⁸ is —C(O)R^{D}. In some embodiments, R⁸ is —C(O)OR^{D}. In some embodiments, R⁸ is —S(O)ₓR^{D}.

In some embodiments, R⁹ is C₁-C₆-alkyl. In some embodiments, R⁹ is C₂-C₆-alkenyl. In some embodiments, R⁹ is C₂-C₆-alkynyl. In some embodiments, R⁹ is C₁-C₆-heteroalkyl. In some embodiments, R⁹ is C₁-C₆-haloalkyl. In some embodiments, R⁹ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁹ is C₁-C₆-alkyl substituted with one or more R¹¹. In some embodiments, R⁹ is C₂-C₆-alkenyl substituted with one or more R¹¹. In some embodiments, R⁹ is C₂-C₆-alkynyl substituted with one or more R¹¹. In some embodiments, R⁹ is C₁-C₆-haloalkyl substituted with one or more R¹¹. In some embodiments, R⁹ is C₁-C₆-heteroalkyl substituted with one or more R¹¹.

In some embodiments, R⁹ is cycloalkyl. In some embodiments, R⁹ is heterocyclyl. In some embodiments, R⁹ is aryl. In some embodiments, R⁹ is heteroaryl. In some embodiments, R⁹ is unsubstituted cycloalkyl, unsubstituted heterocyclyl, unsubstituted aryl, or unsubstituted heteroaryl. In some embodiments, R⁹ is cycloalkyl substituted with one or more R¹¹. In some embodiments, R⁹ is heterocyclyl substituted with one or more R¹¹. In some embodiments, R⁹ is aryl substituted with one or more R¹¹. In some embodiments, R⁹ is heteroaryl substituted with one or more R¹¹.

In some embodiments, R⁹ is halo (e.g., fluoro, chloro, bromo, or iodo). In some embodiments, R⁹ is cyano. In some embodiments, R⁹ is oxo. In some embodiments, R⁹ is —OR^{A}. In some embodiments, R⁹ is —NR^{B}R^{C}. In some embodiments, R⁹ is —NR^{B}C(O)R^{D}. In some embodiments, R⁹ is —NO₂. In some embodiments, R⁹ is —C(O)NR^{B}R^{C}. In some embodiments, R⁹ is —C(O)R^{D}. In some embodiments, R⁹ is —C(O)OR^{D}. In some embodiments, R⁹ is —SR^{E}. In some embodiments, R⁹ is —S(O)ₓR^{D}.

In some embodiments, R¹⁰ is C₁-C₆-alkyl. In some embodiments, R¹⁰ is halo (e.g., fluoro, chloro, bromo, or iodo).

In some embodiments, R¹¹ is C₁-C₆-alkyl. In some embodiments, R¹¹ is C₁-C₆-heteroalkyl. In some embodiments, R¹¹ is C₁-C₆-haloalkyl (e.g., —CF₃). In some embodiments, R¹¹ is cycloalkyl. In some embodiments, R¹¹ is heterocyclyl. In some embodiments, R¹¹ is aryl. In some embodiments, R¹¹ is heteroaryl. In some embodiments, R¹¹ is halo. In some embodiments, R¹¹ is cyano. In some embodiments, R¹¹ is oxo. In some embodiments, R¹¹ is —OR^{A}.

In some embodiments, R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are each independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R¹⁰ (e.g., 1, 2, or 3 R¹⁰).

In some embodiments, R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, R^{D} is hydrogen. In some embodiments, R^{D} is C₁-C₆ alkyl. In some embodiments, R^{D} is C₁-C₆ alkyl (e.g., methyl). In some embodiments, R^{D} is C₁-C₆ heteroalkyl. In some embodiments, R^{D} is C₁-C₆ haloalkyl. In some embodiments, R^{D} is cycloalkyl. In some embodiments, R^{D} is heterocyclyl. In some embodiments, R^{D} is aryl. In some embodiments, R^{D} is heteroaryl. In some embodiments, R^{D} is C₁-C₆ alkylene-aryl (e.g., benzyl). In some embodiments, R^{D} is C₁-C₆ alkylene-heteroaryl.

In some embodiments, m is an integer between 0 and 2 (e.g., 0, 1, or 2). In some embodiments, m is 0. In some embodiments, m is 1. In some embodiments, m is 2. In some embodiments, n is an integer between 0 and 4 (e.g., 0, 1, 2, 3, or 4). In some embodiments, n is 0. In some embodiments, n is 1. In some embodiments, n is 2. In some embodiments, n is 3. In some embodiments, n is 4. In some embodiments, x is an integer between 0 and 2 (e.g., 0, 1, or 2). In some embodiments, x is 0. In some embodiments, x is 1. In some embodiments, x is 2.

In some embodiments, the compound of Formula (I) is a compound of Formula (I-a):

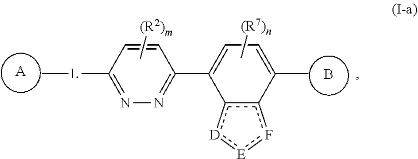

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —S—, —C(O)—, —N(R³)—, —N(R³)C(O)—, or —C(O)N(R³)—, wherein each alkylene and heteroalkylene is optionally substituted with one or more R⁴; D, E, and F are each independently C(R^{5a}), C(R^{5a})(R^{5b}), N, N(R^{5c}), S, or O, wherein at least one of D, E, and F is each independently N or N(R^{5c}), and the bonds between the atoms in the ring comprising D, E, and F may be single bonds or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, C₁-C₆-alkyl, C₂-C₆-alkenyl, C₂-C₆-alkynyl, cycloalkyl, heterocyclyl, halo, cyano, or —OR^{A}; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} and R^{5b} are each independently hydrogen, halo, C₁-C₆ alkyl, or cycloalkyl; or R^{5a} and R^{5b} are taken together to form an oxo group; R^{5c} is hydrogen, C₁-C₆ alkyl, or cycloalkyl; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is independently C₁-C₆-alkyl, C₁-C₆-heteroalkyl, C₁-C₆-haloalkyl, halo, or cycloalkyl; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, or 2; and x is 1, or 2.

In some embodiments, the compound of Formula (I) is a compound of Formula (I-b):

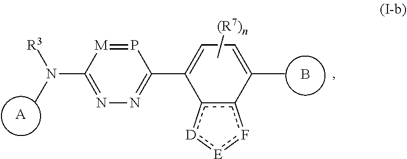

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; M and P are each independently C(R²) or N; D, E, and F are each independently C(R^{5a}) C(R^{5a})(R^{5b}), N, N(R^{5c}), S, or O, wherein at least one of D, E, and F is each independently N or N(R^{5c}), and the bonds between the atoms in the ring comprising D, E, and F may be single bonds or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, C₁-C₆-alkyl, C₂-C₆-alkenyl, C₂-C₆-alkynyl, cycloalkyl, heterocyclyl, halo, cyano, or —OR^{A}; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; R^{5a} and R^{5b} are each independently hydrogen, halo, C₁-C₆ alkyl, or cycloalkyl; or R^{5a} and R^{5b} are taken together to form an oxo group; R^{5c}, is hydrogen, C₁-C₆ alkyl, or cycloalkyl; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹; R¹ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is independently C₁-C₆-alkyl, C₁-C₆-heteroalkyl, C₁-C₆-haloalkyl, halo, or cycloalkyl; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, or 2; and x is 1, or 2.

In some embodiments, the compound of Formula (I) is a compound of Formula (I-c):

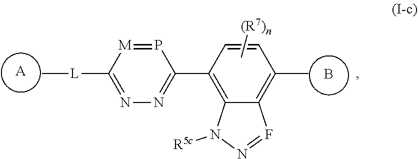

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; M and P are each independently C(R²) or N; F is C(R^{5a}), C(R^{5a})(R^{5b}), N, N(R^{5c}), or S; L is absent, C₁-C₆-alkylene, C₁-C₆-heteroalkylene, —O—, —S—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, C₁-C₆-alkyl, C₂-C₆-alkenyl, C₂-C₆-alkynyl, cycloalkyl, heterocyclyl, halo, cyano, or —OR^{A}; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} and R^{5b} are each independently hydrogen, halo, C₁-C₆ alkyl, or cycloalkyl; or R^{5a} and R^{5b} are taken together to form an oxo group; R^{5c} is hydrogen, C₁-C₆ alkyl, or cycloalkyl; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is independently C₁-C₆-alkyl, C₁-C₆-heteroalkyl, C₁-C₆-haloalkyl, halo, or cycloalkyl; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, or 2; and x is 1, or 2.

In some embodiments, the compound of Formula (I) is a compound of Formula (I-d):

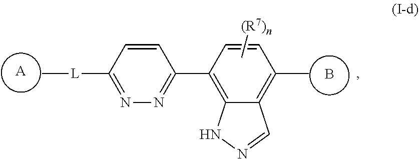

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —S—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is independently C₁-C₆-alkyl, C₁-C₆-heteroalkyl, C₁-C₆-haloalkyl, halo, or cycloalkyl; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, or 2; and x is 1, or 2.

In some embodiments, the compound of Formula (I) is a compound of Formula (I-e):

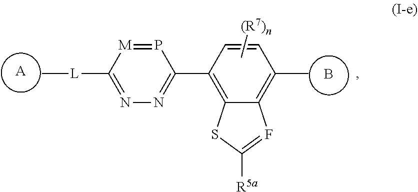

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —S—, —C(O)—, —N(R³)—, —N(R³)C(O)—, or —C(O)N(R³)—, wherein each alkylene and heteroalkylene is optionally substituted with one or more R⁴; M and P are each independently C(R²) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen C₁-C₆-alkyl, C₂-C₆-alkenyl, C₂-C₆-alkynyl, cycloalkyl, heterocyclyl, halo, cyano, or —OR^{A}; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} is hydrogen, halo, C₁-C₆ alkyl, or cycloalkyl; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is independently C₁-C₆-alkyl, C₁-C₆-heteroalkyl, C₁-C₆-haloalkyl, halo, or cycloalkyl; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, or 2; and x is 1, or 2.

In some embodiments, the compound of Formula (I) is a compound of Formula (I-f):

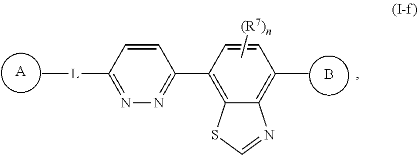

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —S—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is independently C₁-C₆-alkyl, C₁-C₆-heteroalkyl, C₁-C₆-haloalkyl, halo, or cycloalkyl; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, or 2; and x is 1, or 2.

In some embodiments, the compound of Formula (I) is a compound of Formula (I-g):

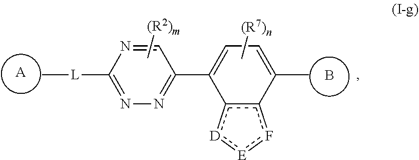

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —S—, —C(O)—, —N(R³)—, —N(R³)C(O)—, or —C(O)N(R³)—, wherein each alkylene and heteroalkylene is optionally substituted with one or more R⁴; D, E, and F are each independently C(R^{5a}), C(R^{5a})(R^{5b}), N, N(R^{5c}), S, or O, wherein at least one of D, E, and F is each independently N or N(R^{5c}), and the bonds between the atoms in the ring comprising D, E, and F may be single bonds or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, C₁-C₆-alkyl, C₂-C₆-alkenyl, C₂-C₆-alkynyl, cycloalkyl, heterocyclyl, halo, cyano, or —OR^{A}; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} and R^{5b} are each independently hydrogen, halo, C₁-C₆ alkyl, or cycloalkyl; or R^{5a} and R^{5b} are taken together to form an oxo group; R^{5c} is hydrogen, C₁-C₆ alkyl, or cycloalkyl; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is independently C₁-C₆-alkyl, C₁-C₆-heteroalkyl, C₁-C₆-haloalkyl, halo, or cycloalkyl; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, or 2; and x is 1, or 2.

In some embodiments, the compound of Formula (I) is a compound of Formula (I-h):

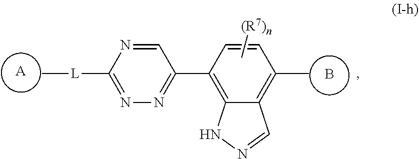

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —S—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is independently C₁-C₆-alkyl, C₁-C₆-heteroalkyl, C₁-C₆-haloalkyl, halo, or cycloalkyl; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, or 2; and x is 1, or 2.

In some embodiments, the compound of Formula (I) is selected from a compound in Table 1, or a pharmaceutically acceptable salt thereof.

TABLE 1
Exemplary compounds

| Compound No. | Structure |
|---|---|
| 100 | 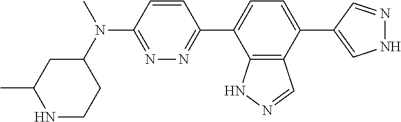 [SMILES: CC1CC(N(C)c2ccc(-c3ccc(-c4cn[nH]c4)c4cn[nH]c34)nn2)CCN1] |
| 101 | 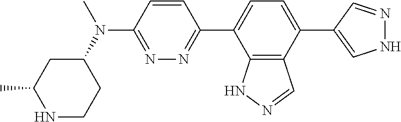 [SMILES: C[C@@H]1C[C@H](N(C)c2ccc(-c3ccc(-c4cn[nH]c4)c4cn[nH]c34)nn2)CCN1] |
| 102 | 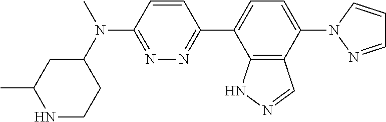 [SMILES: CC1CC(N(C)c2ccc(-c3ccc(-n4cccn4)c4cn[nH]c34)nn2)CCN1] |
| 103 | 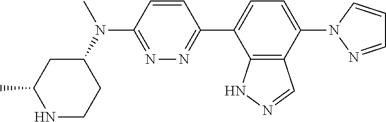 [SMILES: C[C@@H]1C[C@H](N(C)c2ccc(-c3ccc(-n4cccn4)c4cn[nH]c34)nn2)CCN1] |
| 104 | 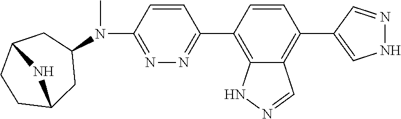 [SMILES: CN(c1ccc(-c2ccc(-c3cn[nH]c3)c3cn[nH]c23)nn1)[C@@H]1C[C@H]2CC[C@@H](C1)N2] |
| 105 | 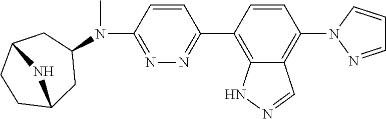 [SMILES: CN(c1ccc(-c2ccc(-n3cccn3)c3cn[nH]c23)nn1)[C@@H]1C[C@H]2CC[C@@H](C1)N2] |
| 106 | 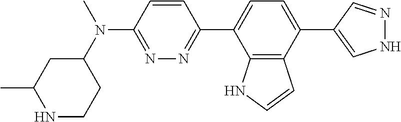 [SMILES: CC1CC(N(C)c2ccc(-c3ccc(-c4cn[nH]c4)c4cc[nH]c34)nn2)CCN1] |
| 107 | 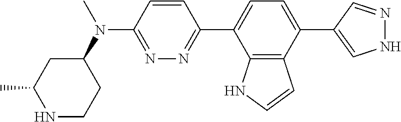 [SMILES: C[C@@H]1C[C@@H](N(C)c2ccc(-c3ccc(-c4cn[nH]c4)c4cc[nH]c34)nn2)CCN1] |
| 108 | 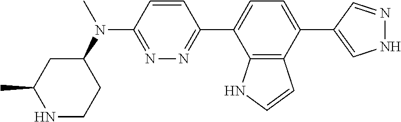 [SMILES: C[C@H]1C[C@@H](N(C)c2ccc(-c3ccc(-c4cn[nH]c4)c4cc[nH]c34)nn2)CCN1] |
| 109 | 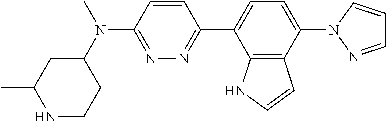 [SMILES: CC1CC(N(C)c2ccc(-c3ccc(-n4cccn4)c4cc[nH]c34)nn2)CCN1] |
| 110 | 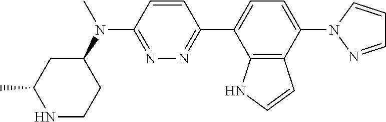 [SMILES: C[C@@H]1C[C@@H](N(C)c2ccc(-c3ccc(-n4cccn4)c4cc[nH]c34)nn2)CCN1] |
| 111 | 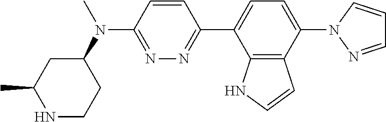 [SMILES: C[C@H]1C[C@@H](N(C)c2ccc(-c3ccc(-n4cccn4)c4cc[nH]c34)nn2)CCN1] |
| 112 | 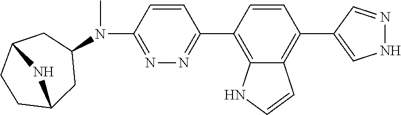 [SMILES: CN(c1ccc(-c2ccc(-c3cn[nH]c3)c3cc[nH]c23)nn1)[C@@H]1C[C@H]2CC[C@@H](C1)N2] |
| 113 | 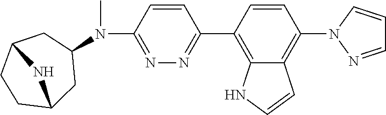 [SMILES: CN(c1ccc(-c2ccc(-n3cccn3)c3cc[nH]c23)nn1)[C@@H]1C[C@H]2CC[C@@H](C1)N2] |
| 114 | 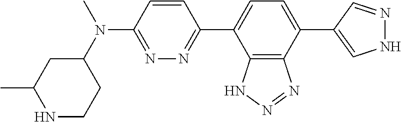 [SMILES: CC1CC(N(C)c2ccc(-c3ccc(-c4cn[nH]c4)c4nn[nH]c34)nn2)CCN1] |
| 115 | 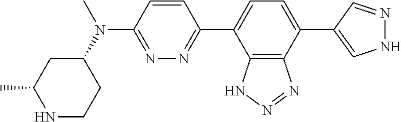 [SMILES: C[C@@H]1C[C@H](N(C)c2ccc(-c3ccc(-c4cn[nH]c4)c4nn[nH]c34)nn2)CCN1] |
| 116 | 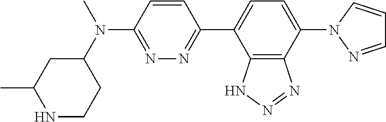 [SMILES: CC1CC(N(C)c2ccc(-c3ccc(-n4cccn4)c4nn[nH]c34)nn2)CCN1] |
| 117 | 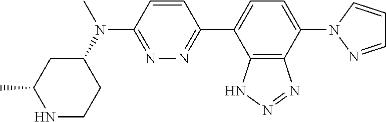 [SMILES: C[C@@H]1C[C@H](N(C)c2ccc(-c3ccc(-n4cccn4)c4nn[nH]c34)nn2)CCN1] |
| 118 | 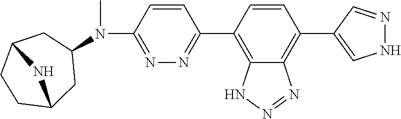 [SMILES: CN(c1ccc(-c2ccc(-c3cn[nH]c3)c3nn[nH]c23)nn1)[C@@H]1C[C@H]2CC[C@@H](C1)N2] |
| 119 | 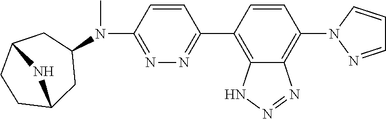 [SMILES: CN(c1ccc(-c2ccc(-n3cccn3)c3nn[nH]c23)nn1)[C@@H]1C[C@H]2CC[C@@H](C1)N2] |
| 120 | 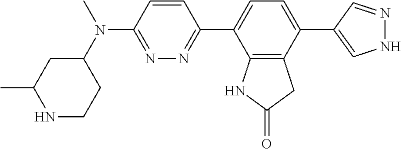 [SMILES: CC1CC(N(C)c2ccc(-c3ccc(-c4cn[nH]c4)c4c3NC(=O)C4)nn2)CCN1] |
| 121 | 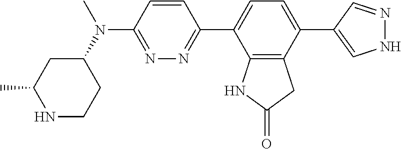 [SMILES: C[C@@H]1C[C@H](N(C)c2ccc(-c3ccc(-c4cn[nH]c4)c4c3NC(=O)C4)nn2)CCN1] |
| 122 | 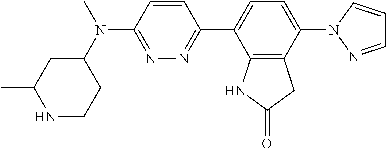 [SMILES: CC1CC(N(C)c2ccc(-c3ccc(-n4cccn4)c4c3NC(=O)C4)nn2)CCN1] |
| 123 | 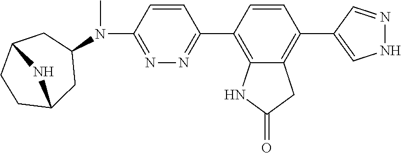 [SMILES: CN(c1ccc(-c2ccc(-c3cn[nH]c3)c3c2NC(=O)C3)nn1)[C@@H]1C[C@H]2CC[C@@H](C1)N2] |
| 124 | 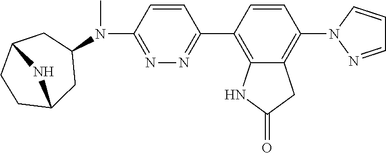 [SMILES: CN(c1ccc(-c2ccc(-n3cccn3)c3c2NC(=O)C3)nn1)[C@@H]1C[C@H]2CC[C@@H](C1)N2] |
| 131 | 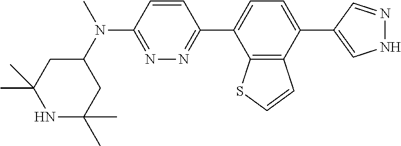 [SMILES: CN(c1ccc(-c2ccc(-c3cn[nH]c3)c3ccsc23)nn1)C1CC(C)(C)NC(C)(C)C1] |
| 132 | 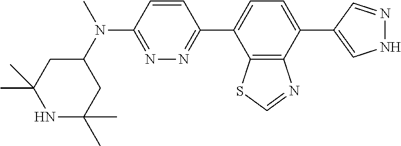 [SMILES: CN(c1ccc(-c2ccc(-c3cn[nH]c3)c3ncsc23)nn1)C1CC(C)(C)NC(C)(C)C1] |
| 137 | 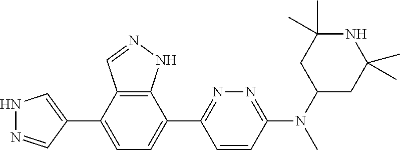 [SMILES: CN(c1ccc(-c2ccc(-c3cn[nH]c3)c3cn[nH]c23)nn1)C1CC(C)(C)NC(C)(C)C1] |
| 138 | 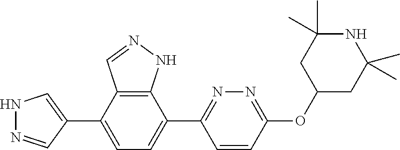 [SMILES: CC1(C)CC(Oc2ccc(-c3ccc(-c4cn[nH]c4)c4cn[nH]c34)nn2)CC(C)(C)N1] |
| 139 | 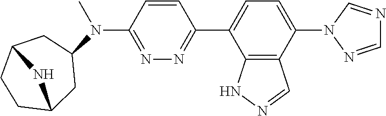 [SMILES: CN(c1ccc(-c2ccc(-n3cncn3)c3cn[nH]c23)nn1)[C@@H]1C[C@H]2CC[C@@H](C1)N2] |
| 140 | 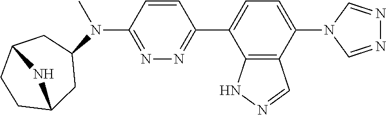 [SMILES: CN(c1ccc(-c2ccc(-n3cnnc3)c3cn[nH]c23)nn1)[C@@H]1C[C@H]2CC[C@@H](C1)N2] |
| 141 | 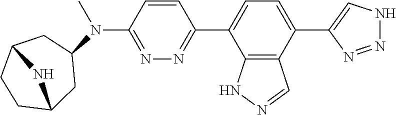 [SMILES: CN(c1ccc(-c2ccc(-c3c[nH]nn3)c3cn[nH]c23)nn1)[C@@H]1C[C@H]2CC[C@@H](C1)N2] |
| 142 | 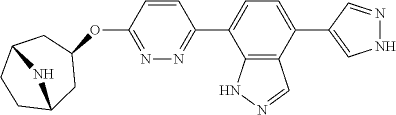 [SMILES: c1n[nH]cc1-c1ccc(-c2ccc(O[C@@H]3C[C@H]4CC[C@@H](C3)N4)nn2)c2[nH]ncc12] |
| 144 | 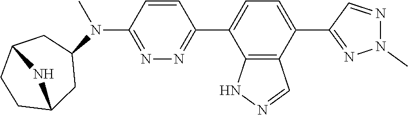 [SMILES: CN(c1ccc(-c2ccc(-c3cnn(C)n3)c3cn[nH]c23)nn1)[C@@H]1C[C@H]2CC[C@@H](C1)N2] |
| 145 | 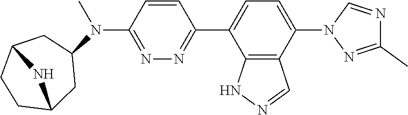 [SMILES: Cc1ncn(-c2ccc(-c3ccc(N(C)[C@@H]4C[C@H]5CC[C@@H](C4)N5)nn3)c3[nH]ncc23)n1] |
| 146 | 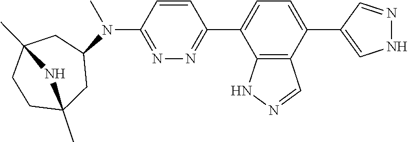 [SMILES: CN(c1ccc(-c2ccc(-c3cn[nH]c3)c3cn[nH]c23)nn1)[C@H]1C[C@]2(C)CC[C@](C)(C1)N2] |
| 147 | 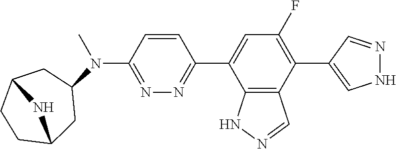 [SMILES: CN(c1ccc(-c2cc(F)c(-c3cn[nH]c3)c3cn[nH]c23)nn1)[C@@H]1C[C@H]2CC[C@@H](C1)N2] |
| 148 | 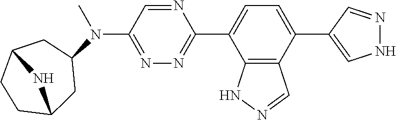 [SMILES: CN(c1cnc(-c2ccc(-c3cn[nH]c3)c3cn[nH]c23)nn1)[C@@H]1C[C@H]2CC[C@@H](C1)N2] |
| 149 | 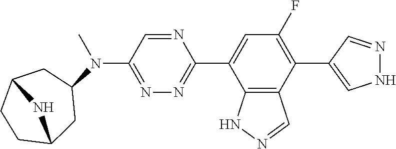 [SMILES: CN(c1cnc(-c2cc(F)c(-c3cn[nH]c3)c3cn[nH]c23)nn1)[C@@H]1C[C@H]2CC[C@@H](C1)N2] |
| 150 | 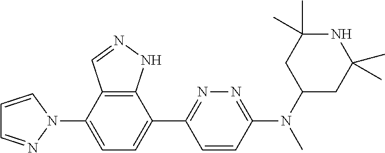 [SMILES: CN(c1ccc(-c2ccc(-n3cccn3)c3cn[nH]c23)nn1)C1CC(C)(C)NC(C)(C)C1] |
| 151 | 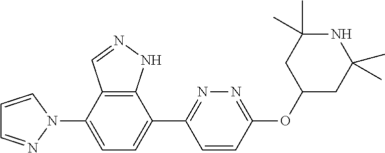 [SMILES: CC1(C)CC(Oc2ccc(-c3ccc(-n4cccn4)c4cn[nH]c34)nn2)CC(C)(C)N1] |
| 152 | 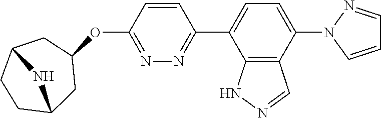 [SMILES: c1cnn(-c2ccc(-c3ccc(O[C@@H]4C[C@H]5CC[C@@H](C4)N5)nn3)c3[nH]ncc23)c1] |
| 153 | 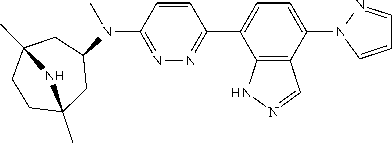 [SMILES: CN(c1ccc(-c2ccc(-n3cccn3)c3cn[nH]c23)nn1)[C@H]1C[C@]2(C)CC[C@](C)(C1)N2] |
| 154 | 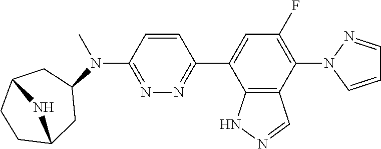 [SMILES: CN(c1ccc(-c2cc(F)c(-n3cccn3)c3cn[nH]c23)nn1)[C@@H]1C[C@H]2CC[C@@H](C1)N2] |
| 155 | 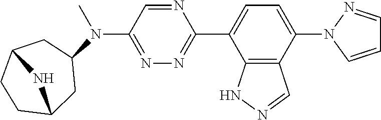 [SMILES: CN(c1cnc(-c2ccc(-n3cccn3)c3cn[nH]c23)nn1)[C@@H]1C[C@H]2CC[C@@H](C1)N2] |
| 156 | 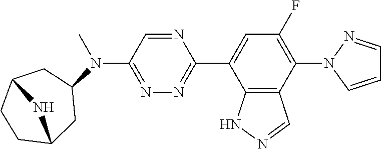 [SMILES: CN(c1cnc(-c2cc(F)c(-n3cccn3)c3cn[nH]c23)nn1)[C@@H]1C[C@H]2CC[C@@H](C1)N2] |
| 157 | 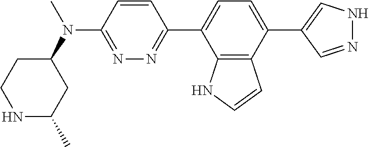 [SMILES: C[C@H]1C[C@H](N(C)c2ccc(-c3ccc(-c4cn[nH]c4)c4cc[nH]c34)nn2)CCN1] |
| 158 | 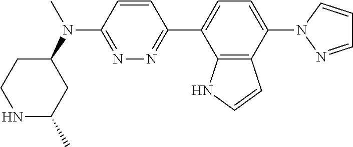 [SMILES: C[C@H]1C[C@H](N(C)c2ccc(-c3ccc(-n4cccn4)c4cc[nH]c34)nn2)CCN1] |
| 159 | 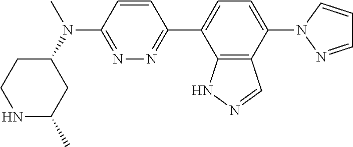 [SMILES: C[C@H]1C[C@@H](N(C)c2ccc(-c3ccc(-n4cccn4)c4cn[nH]c34)nn2)CCN1] |
| 160 | 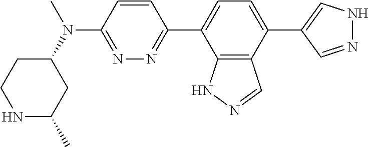 [SMILES: C[C@H]1C[C@@H](N(C)c2ccc(-c3ccc(-c4cn[nH]c4)c4cn[nH]c34)nn2)CCN1] |
| 163 | 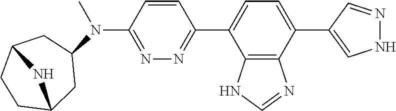 [SMILES: CN(c1ccc(-c2ccc(-c3cn[nH]c3)c3nc[nH]c23)nn1)[C@@H]1C[C@H]2CC[C@@H](C1)N2] |
| 164 | 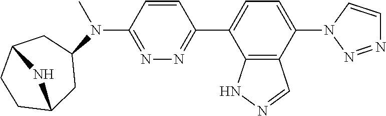 [SMILES: CN(c1ccc(-c2ccc(-n3ccnn3)c3cn[nH]c23)nn1)[C@@H]1C[C@H]2CC[C@@H](C1)N2] |
| 165 | 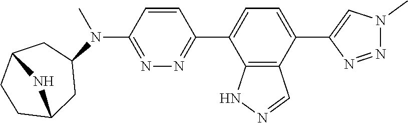 [SMILES: CN(c1ccc(-c2ccc(-c3cn(C)nn3)c3cn[nH]c23)nn1)[C@@H]1C[C@H]2CC[C@@H](C1)N2] |
| 168 | 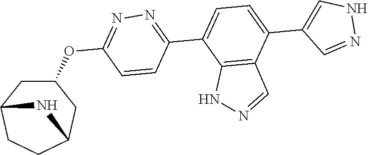 [SMILES: c1n[nH]cc1-c1ccc(-c2ccc(O[C@H]3C[C@H]4CC[C@@H](C3)N4)nn2)c2[nH]ncc12] |
| 169 | 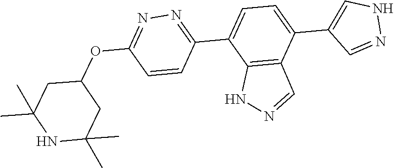 [SMILES: CC1(C)CC(Oc2ccc(-c3ccc(-c4cn[nH]c4)c4cn[nH]c34)nn2)CC(C)(C)N1] |
| 170 | 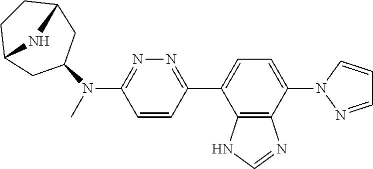 [SMILES: CN(c1ccc(-c2ccc(-n3cccn3)c3nc[nH]c23)nn1)[C@@H]1C[C@H]2CC[C@@H](C1)N2] |
| 174 | 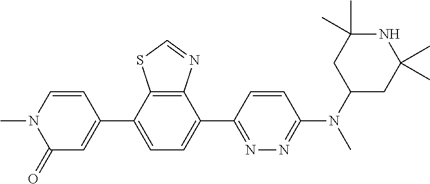 |
| 175 | 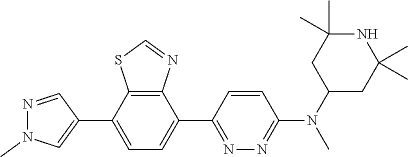 |
| 176 | 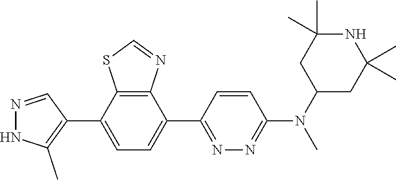 [SMILES: Cc1[nH]ncc1-c1ccc(-c2ccc(N(C)C3CC(C)(C)NC(C)(C)C3)nn2)c2ncsc12] |
| 177 | 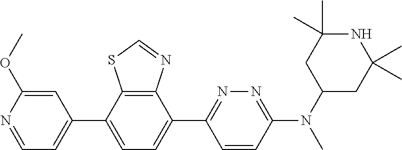 [SMILES: COc1cc(-c2ccc(-c3ccc(N(C)C4CC(C)(C)NC(C)(C)C4)nn3)c3ncsc23)ccn1] |
| 178 | 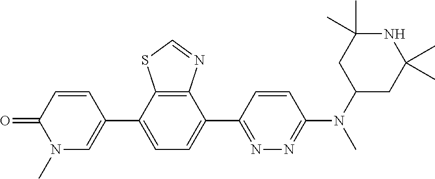 [SMILES: CN(c1ccc(-c2ccc(-c3ccc(=O)n(C)c3)c3scnc23)nn1)C1CC(C)(C)NC(C)(C)C1] |
| 179 | 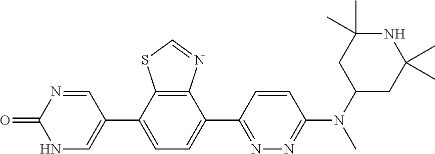 [SMILES: CN(c1ccc(-c2ccc(-c3cnc(=O)[nH]c3)c3scnc23)nn1)C1CC(C)(C)NC(C)(C)C1] |
| 180 | 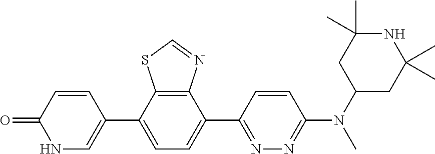 [SMILES: CN(c1ccc(-c2ccc(-c3ccc(=O)[nH]c3)c3scnc23)nn1)C1CC(C)(C)NC(C)(C)C1] |
| 181 | 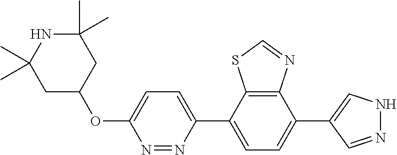 [SMILES: CC1(C)CC(Oc2ccc(-c3ccc(-c4cn[nH]c4)c4ncsc34)nn2)CC(C)(C)N1] |
| 182 | 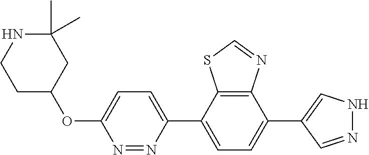 [SMILES: CC1(C)CC(Oc2ccc(-c3ccc(-c4cn[nH]c4)c4ncsc34)nn2)CCN1] |
| 183 | 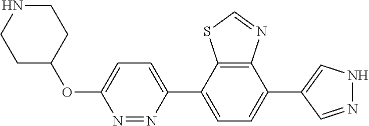 [SMILES: c1nc2c(-c3cn[nH]c3)ccc(-c3ccc(OC4CCNCC4)nn3)c2s1] |
| 184 | 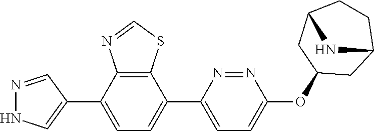 [SMILES: c1nc2c(-c3cn[nH]c3)ccc(-c3ccc(O[C@@H]4C[C@H]5CC[C@@H](C4)N5)nn3)c2s1] |
| 185 | 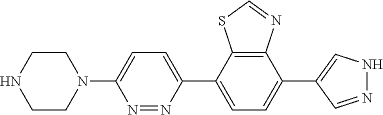 [SMILES: c1nc2c(-c3cn[nH]c3)ccc(-c3ccc(N4CCNCC4)nn3)c2s1] |
| 186 | 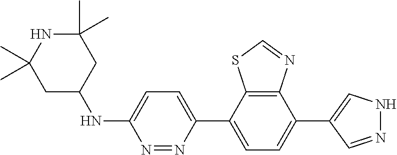 [SMILES: CC1(C)CC(Nc2ccc(-c3ccc(-c4cn[nH]c4)c4ncsc34)nn2)CC(C)(C)N1] |
| 187 | 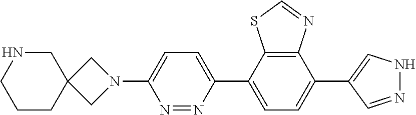 [SMILES: c1nc2c(-c3cn[nH]c3)ccc(-c3ccc(N4CC5(CCCNC5)C4)nn3)c2s1] |
| 188 | 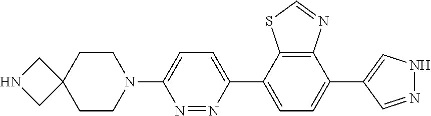 [SMILES: c1nc2c(-c3cn[nH]c3)ccc(-c3ccc(N4CCC5(CC4)CNC5)nn3)c2s1] |
| 189 | 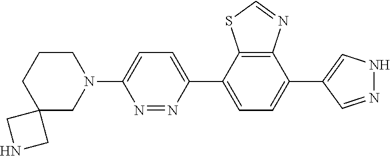 [SMILES: c1nc2c(-c3cn[nH]c3)ccc(-c3ccc(N4CCCC5(CNC5)C4)nn3)c2s1] |
| 190 | 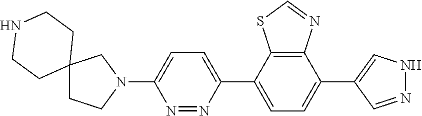 [SMILES: c1nc2c(-c3cn[nH]c3)ccc(-c3ccc(N4CCC5(CCNCC5)C4)nn3)c2s1] |
| 191 | 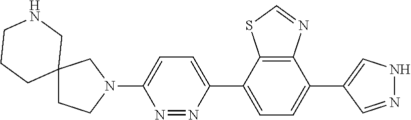 [SMILES: c1nc2c(-c3cn[nH]c3)ccc(-c3ccc(N4CCC5(CCCNC5)C4)nn3)c2s1] |
| 192 | 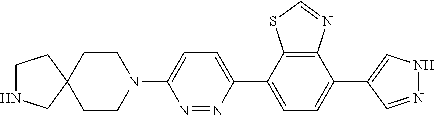 [SMILES: c1nc2c(-c3cn[nH]c3)ccc(-c3ccc(N4CCC5(CCNC5)CC4)nn3)c2s1] |
| 193 | 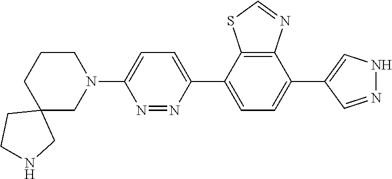 [SMILES: c1nc2c(-c3cn[nH]c3)ccc(-c3ccc(N4CCCC5(CCNC5)C4)nn3)c2s1] |
| 194 | 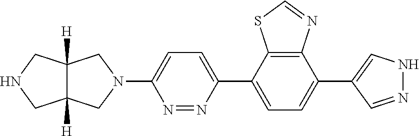 [SMILES: c1nc2c(-c3cn[nH]c3)ccc(-c3ccc(N4C[C@H]5CNC[C@H]5C4)nn3)c2s1] |
| 196 | 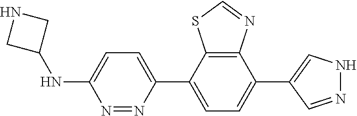 [SMILES: c1nc2c(-c3cn[nH]c3)ccc(-c3ccc(NC4CNC4)nn3)c2s1] |
| 197 | 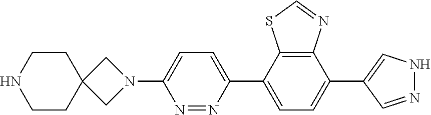 [SMILES: c1nc2c(-c3cn[nH]c3)ccc(-c3ccc(N4CC5(CCNCC5)C4)nn3)c2s1] |
| 198 | 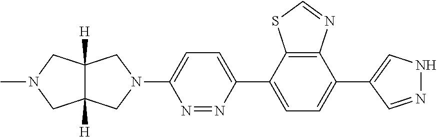 [SMILES: CN1C[C@@H]2CN(c3ccc(-c4ccc(-c5cn[nH]c5)c5ncsc45)nn3)C[C@@H]2C1] |
| 199 | 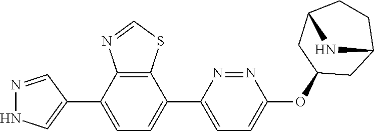 [SMILES: c1nc2c(-c3cn[nH]c3)ccc(-c3ccc(O[C@@H]4C[C@H]5CC[C@@H](C4)N5)nn3)c2s1] |
| 200 | 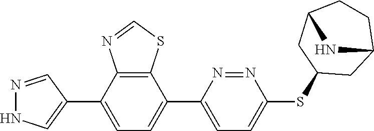 [SMILES: c1nc2c(-c3cn[nH]c3)ccc(-c3ccc(S[C@@H]4C[C@H]5CC[C@@H](C4)N5)nn3)c2s1] |
| 201 | 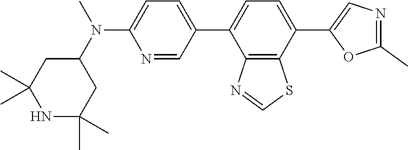 |
| 202 | 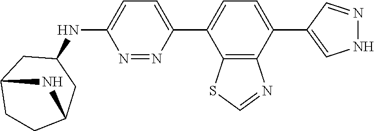 [SMILES: c1nc2c(-c3cn[nH]c3)ccc(-c3ccc(N[C@@H]4C[C@H]5CC[C@@H](C4)N5)nn3)c2s1] |
| 203 | 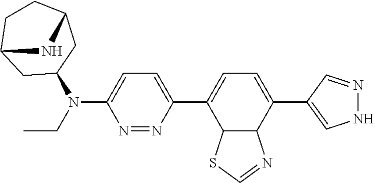 [SMILES: CCN(c1ccc(C2=CC=C(c3cn[nH]c3)C3N=CSC23)nn1)[C@@H]1C[C@H]2CC[C@@H](C1)N2] |
| 204 | 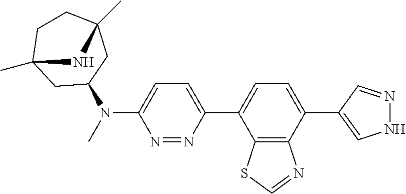 [SMILES: CN(c1ccc(-c2ccc(-c3cn[nH]c3)c3ncsc23)nn1)[C@H]1C[C@]2(C)CC[C@](C)(C1)N2] |
| 205 | 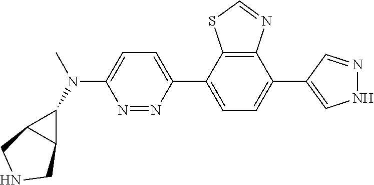 [SMILES: CN(c1ccc(-c2ccc(-c3cn[nH]c3)c3ncsc23)nn1)[C@H]1[C@@H]2CNC[C@@H]21] |
| 206 | 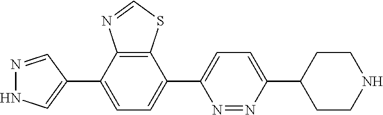 [SMILES: c1nc2c(-c3cn[nH]c3)ccc(-c3ccc(C4CCNCC4)nn3)c2s1] |
| 209 | 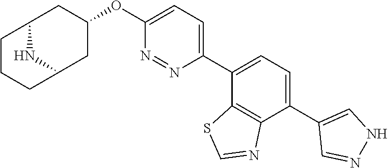 [SMILES: c1nc2c(-c3cn[nH]c3)ccc(-c3ccc(O[C@@H]4C[C@H]5CCC[C@@H](C4)N5)nn3)c2s1] |
| 210 | 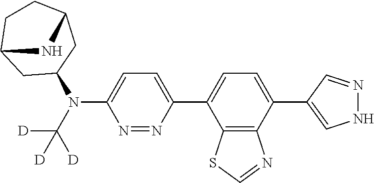 [SMILES: [2H]C([2H])([2H])N(c1ccc(-c2ccc(-c3cn[nH]c3)c3ncsc23)nn1)[C@@H]1C[C@H]2CC[C@@H](C1)N2] |
| 211 | 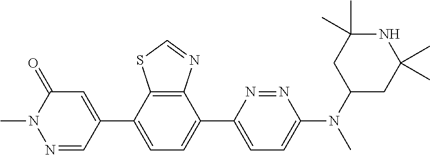 |
| 212 | 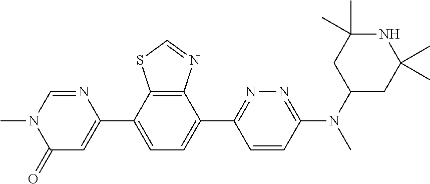 |
| 213 | 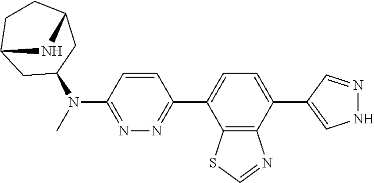 [SMILES: CN(c1ccc(-c2ccc(-c3cn[nH]c3)c3ncsc23)nn1)[C@@H]1C[C@H]2CC[C@@H](C1)N2] |
| 214 | 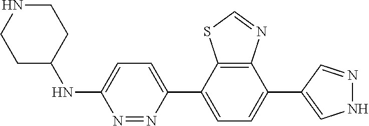 [SMILES: c1nc2c(-c3cn[nH]c3)ccc(-c3ccc(NC4CCNCC4)nn3)c2s1] |
| 215 | 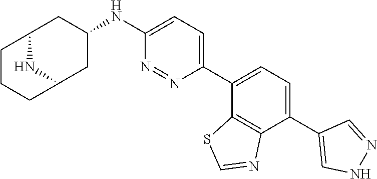 [SMILES: c1nc2c(-c3cn[nH]c3)ccc(-c3ccc(N[C@@H]4C[C@H]5CCC[C@@H](C4)N5)nn3)c2s1] |
| 216 | 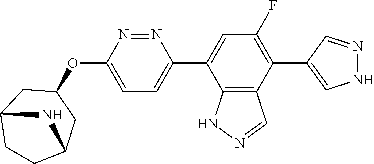 [SMILES: Fc1cc(-c2ccc(O[C@@H]3C[C@H]4CC[C@@H](C3)N4)nn2)c2[nH]ncc2c1-c1cn[nH]c1] |
| 217 | 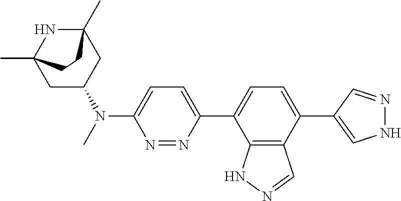 [SMILES: CN(c1ccc(-c2ccc(-c3cn[nH]c3)c3cn[nH]c23)nn1)[C@H]1C[C@]2(C)CC[C@](C)(C1)N2] |
| 218 | 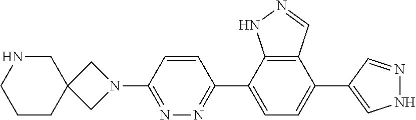 [SMILES: c1n[nH]cc1-c1ccc(-c2ccc(N3CC4(CCCNC4)C3)nn2)c2[nH]ncc12] |
| 219 | 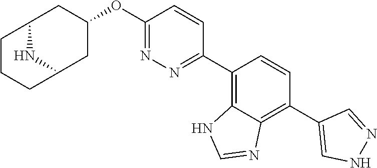 [SMILES: c1nc2c(-c3cn[nH]c3)ccc(-c3ccc(O[C@@H]4C[C@H]5CCC[C@@H](C4)N5)nn3)c2[nH]1] |
| 220 | 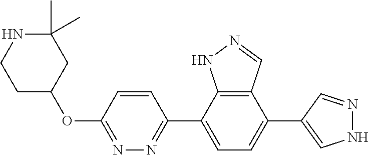 [SMILES: CC1(C)CC(Oc2ccc(-c3ccc(-c4cn[nH]c4)c4cn[nH]c34)nn2)CCN1] |
| 221 | 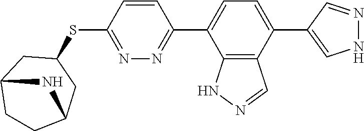 [SMILES: c1n[nH]cc1-c1ccc(-c2ccc(S[C@@H]3C[C@H]4CC[C@@H](C3)N4)nn2)c2[nH]ncc12] |
| 222 | 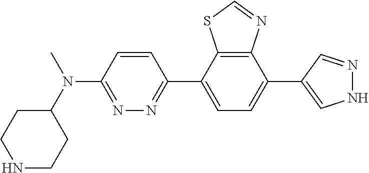 [SMILES: CN(c1ccc(-c2ccc(-c3cn[nH]c3)c3ncsc23)nn1)C1CCNCC1] |
| 223 | 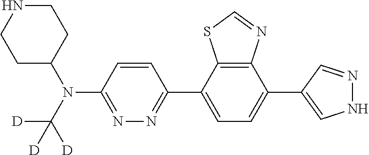 [SMILES: [2H]C([2H])([2H])N(c1ccc(-c2ccc(-c3cn[nH]c3)c3ncsc23)nn1)C1CCNCC1] |
| 224 | 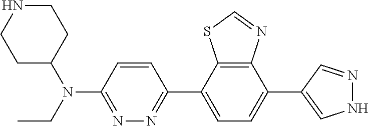 [SMILES: CCN(c1ccc(-c2ccc(-c3cn[nH]c3)c3ncsc23)nn1)C1CCNCC1] |
| 225 | 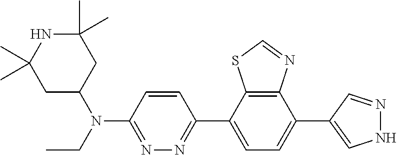 [SMILES: CCN(c1ccc(-c2ccc(-c3cn[nH]c3)c3ncsc23)nn1)C1CC(C)(C)NC(C)(C)C1] |
| 226 | 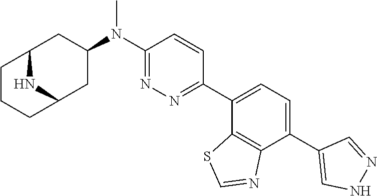 [SMILES: CN(c1ccc(-c2ccc(-c3cn[nH]c3)c3ncsc23)nn1)[C@@H]1C[C@H]2CCC[C@@H](C1)N2] |
| 227 | 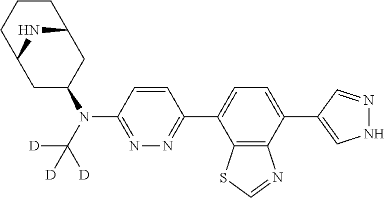 [SMILES: [2H]C([2H])([2H])N(c1ccc(-c2ccc(-c3cn[nH]c3)c3ncsc23)nn1)[C@@H]1C[C@H]2CCC[C@@H](C1)N2] |
| 228 | 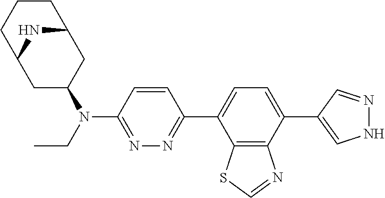 [SMILES: CCN(c1ccc(-c2ccc(-c3cn[nH]c3)c3ncsc23)nn1)[C@@H]1C[C@H]2CCC[C@@H](C1)N2] |
| 229 | 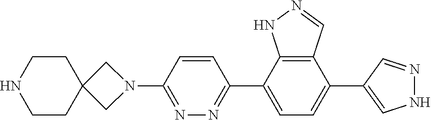 [SMILES: c1n[nH]cc1-c1ccc(-c2ccc(N3CC4(CCNCC4)C3)nn2)c2[nH]ncc12] |
| 230 | 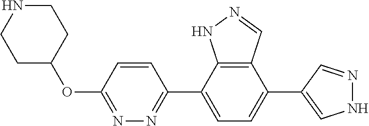 [SMILES: c1n[nH]cc1-c1ccc(-c2ccc(OC3CCNCC3)nn2)c2[nH]ncc12] |
| 231 | 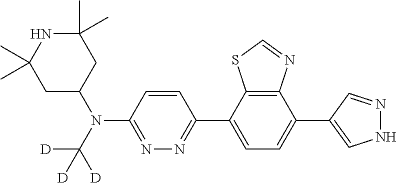 [SMILES: [2H]C([2H])([2H])N(c1ccc(-c2ccc(-c3cn[nH]c3)c3ncsc23)nn1)C1CC(C)(C)NC(C)(C)C1] |
| 232 | 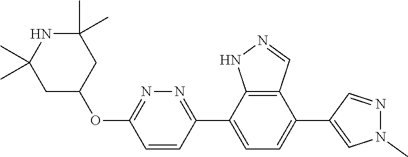 |
| 233 | 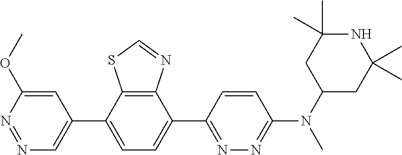 [SMILES: COc1cc(-c2ccc(-c3ccc(N(C)C4CC(C)(C)NC(C)(C)C4)nn3)c3ncsc23)cnn1] |
| 234 | 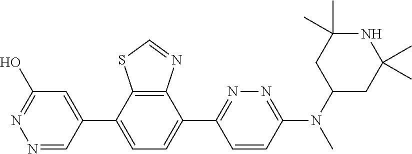 [SMILES: CN(c1ccc(-c2ccc(-c3cnnc(O)c3)c3scnc23)nn1)C1CC(C)(C)NC(C)(C)C1] |
| 235 | 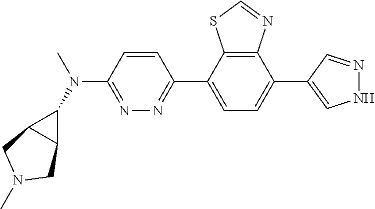 [SMILES: CN1C[C@@H]2[C@H](C1)[C@H]2N(C)c1ccc(-c2ccc(-c3cn[nH]c3)c3ncsc23)nn1] |
| 237 | 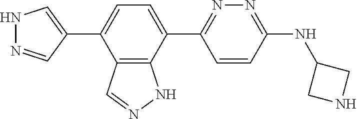 [SMILES: c1n[nH]cc1-c1ccc(-c2ccc(NC3CNC3)nn2)c2[nH]ncc12] |
| 238 | 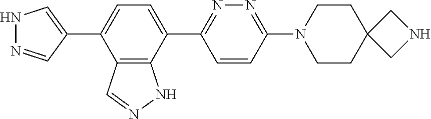 [SMILES: c1n[nH]cc1-c1ccc(-c2ccc(N3CCC4(CC3)CNC4)nn2)c2[nH]ncc12] |
| 239 | 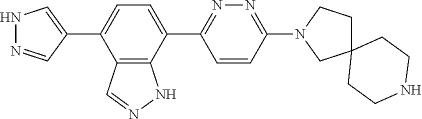 [SMILES: c1n[nH]cc1-c1ccc(-c2ccc(N3CCC4(CCNCC4)C3)nn2)c2[nH]ncc12] |
| 240 | 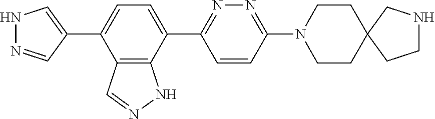 [SMILES: c1n[nH]cc1-c1ccc(-c2ccc(N3CCC4(CCNC4)CC3)nn2)c2[nH]ncc12] |
| 241 | 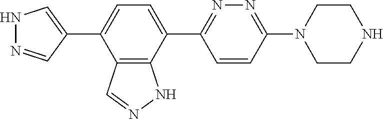 [SMILES: c1n[nH]cc1-c1ccc(-c2ccc(N3CCNCC3)nn2)c2[nH]ncc12] |
| 242 | 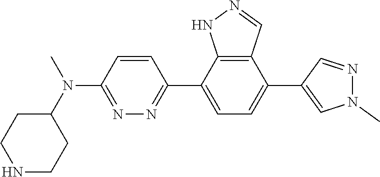 [SMILES: CN(c1ccc(-c2ccc(-c3cnn(C)c3)c3cn[nH]c23)nn1)C1CCNCC1] |
| 243 | 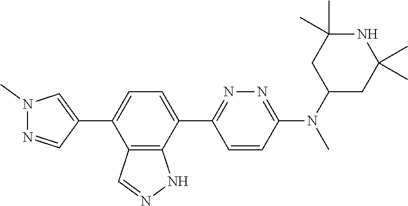 |
| 251 | 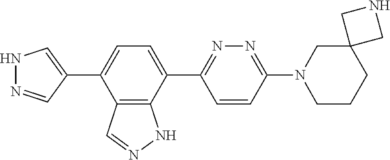 [SMILES: c1n[nH]cc1-c1ccc(-c2ccc(N3CCCC4(CNC4)C3)nn2)c2[nH]ncc12] |
| 252 | 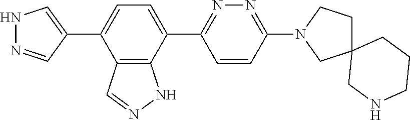 [SMILES: c1n[nH]cc1-c1ccc(-c2ccc(N3CCC4(CCCNC4)C3)nn2)c2[nH]ncc12] |
| 253 | 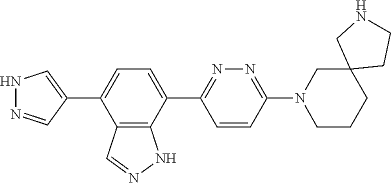 [SMILES: c1n[nH]cc1-c1ccc(-c2ccc(N3CCCC4(CCNC4)C3)nn2)c2[nH]ncc12] |
| 254 | 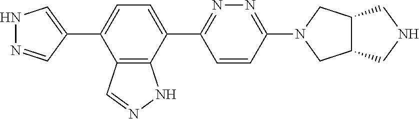 [SMILES: c1n[nH]cc1-c1ccc(-c2ccc(N3C[C@H]4CNC[C@H]4C3)nn2)c2[nH]ncc12] |
| 255 | 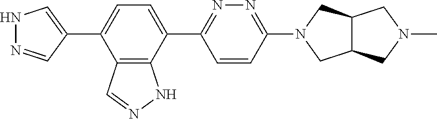 [SMILES: CN1C[C@@H]2CN(c3ccc(-c4ccc(-c5cn[nH]c5)c5cn[nH]c45)nn3)C[C@@H]2C1] |
| 263 | 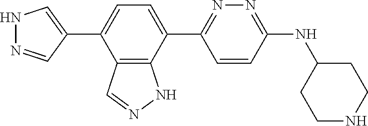 [SMILES: c1n[nH]cc1-c1ccc(-c2ccc(NC3CCNCC3)nn2)c2[nH]ncc12] |
| 264 | 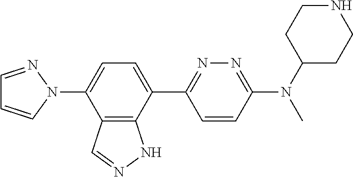 [SMILES: CN(c1ccc(-c2ccc(-n3cccn3)c3cn[nH]c23)nn1)C1CCNCC1] |
| 265 | 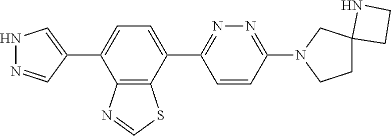 [SMILES: c1nc2c(-c3cn[nH]c3)ccc(-c3ccc(N4CCC5(CCN5)C4)nn3)c2s1] |
| 266 | 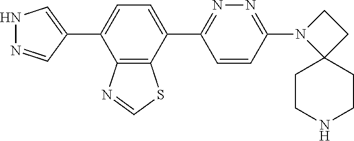 [SMILES: c1nc2c(-c3cn[nH]c3)ccc(-c3ccc(N4CCC45CCNCC5)nn3)c2s1] |
| 267 | 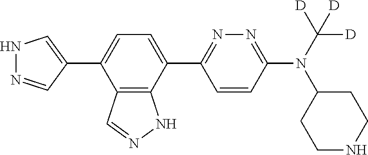 [SMILES: [2H]C([2H])([2H])N(c1ccc(-c2ccc(-c3cn[nH]c3)c3cn[nH]c23)nn1)C1CCNCC1] |
| 273 | 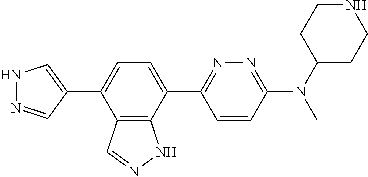 [SMILES: CN(c1ccc(-c2ccc(-c3cn[nH]c3)c3cn[nH]c23)nn1)C1CCNCC1] |
| 274 | 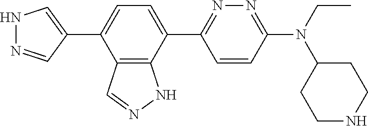 [SMILES: CCN(c1ccc(-c2ccc(-c3cn[nH]c3)c3cn[nH]c23)nn1)C1CCNCC1] |
| 275 | 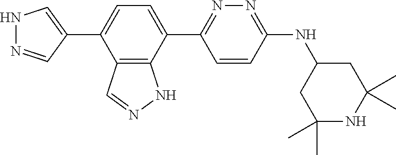 [SMILES: CC1(C)CC(Nc2ccc(-c3ccc(-c4cn[nH]c4)c4cn[nH]c34)nn2)CC(C)(C)N1] |
| 276 | 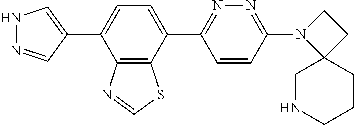 [SMILES: c1nc2c(-c3cn[nH]c3)ccc(-c3ccc(N4CCC45CCCNC5)nn3)c2s1] |
| 277 | 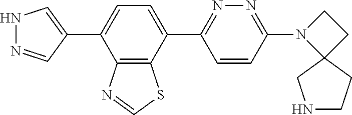 [SMILES: c1nc2c(-c3cn[nH]c3)ccc(-c3ccc(N4CCC45CCNC5)nn3)c2s1] |
| 278 | 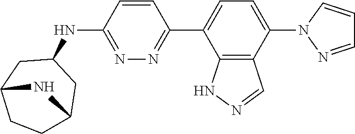 [SMILES: c1cnn(-c2ccc(-c3ccc(N[C@@H]4C[C@H]5CC[C@@H](C4)N5)nn3)c3[nH]ncc23)c1] |
| 279 | 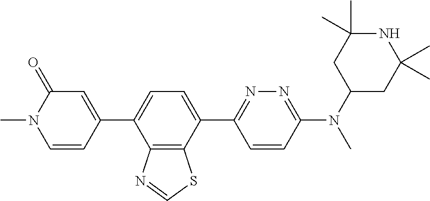 |
| 280 | 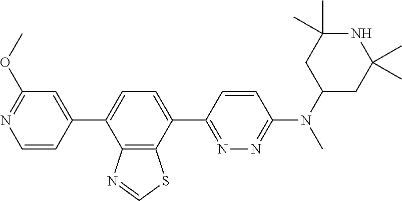 [SMILES: COc1cc(-c2ccc(-c3ccc(N(C)C4CC(C)(C)NC(C)(C)C4)nn3)c3scnc23)ccn1] |
| 281 | 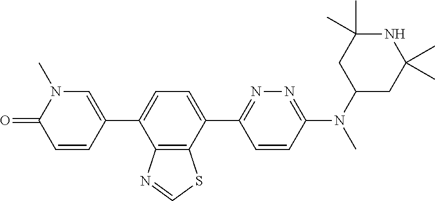 [SMILES: CN(c1ccc(-c2ccc(-c3ccc(=O)n(C)c3)c3ncsc23)nn1)C1CC(C)(C)NC(C)(C)C1] |
| 282 | 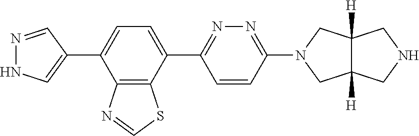 [SMILES: c1nc2c(-c3cn[nH]c3)ccc(-c3ccc(N4C[C@H]5CNC[C@H]5C4)nn3)c2s1] |
| 283 | 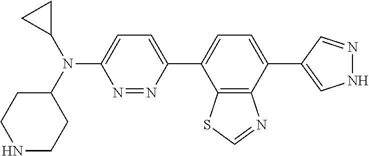 [SMILES: c1nc2c(-c3cn[nH]c3)ccc(-c3ccc(N(C4CCNCC4)C4CC4)nn3)c2s1] |
| 284 | 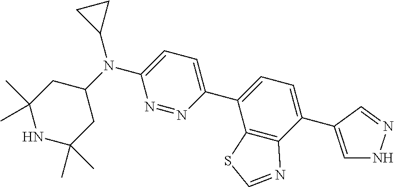 [SMILES: CC1(C)CC(N(c2ccc(-c3ccc(-c4cn[nH]c4)c4ncsc34)nn2)C2CC2)CC(C)(C)N1] |
| 285 | 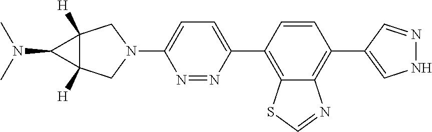 [SMILES: CN(C)[C@H]1[C@@H]2CN(c3ccc(-c4ccc(-c5cn[nH]c5)c5ncsc45)nn3)C[C@@H]21] |
| 286 | 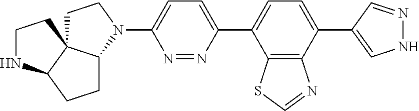 [SMILES: c1nc2c(-c3cn[nH]c3)ccc(-c3ccc(N4CC[C@@]56CCN[C@@H]5CC[C@@H]46)nn3)c2s1] |
| 287 | 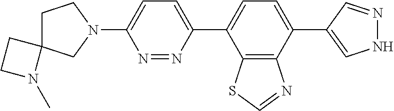 [SMILES: CN1CCC12CCN(c1ccc(-c3ccc(-c4cn[nH]c4)c4ncsc34)nn1)C2] |
| 288 | 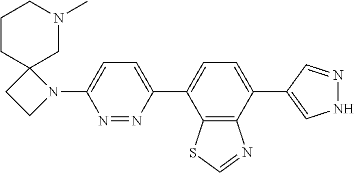 [SMILES: CN1CCCC2(CCN2c2ccc(-c3ccc(-c4cn[nH]c4)c4ncsc34)nn2)C1] |
| 289 | 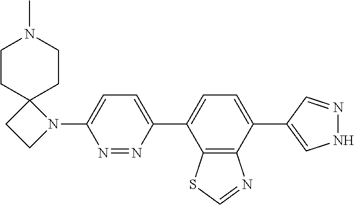 [SMILES: CN1CCC2(CC1)CCN2c1ccc(-c2ccc(-c3cn[nH]c3)c3ncsc23)nn1] |
| 290 | 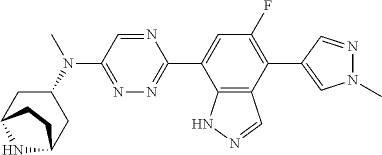 |
| 291 | 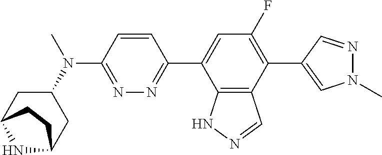 |
| 292 | 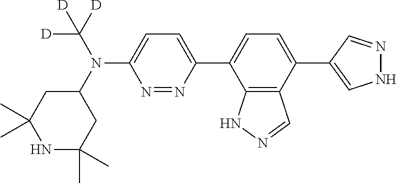 [SMILES: [2H]C([2H])([2H])N(c1ccc(-c2ccc(-c3cn[nH]c3)c3cn[nH]c23)nn1)C1CC(C)(C)NC(C)(C)C1] |
| 293 | 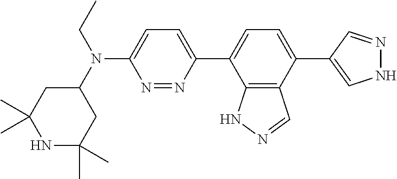 [SMILES: CCN(c1ccc(-c2ccc(-c3cn[nH]c3)c3cn[nH]c23)nn1)C1CC(C)(C)NC(C)(C)C1] |
| 294 | 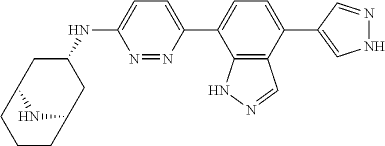 [SMILES: c1n[nH]cc1-c1ccc(-c2ccc(N[C@@H]3C[C@H]4CCC[C@@H](C3)N4)nn2)c2[nH]ncc12] |
| 295 | 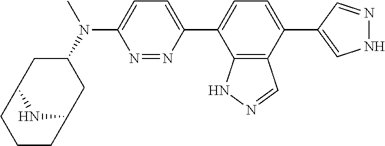 [SMILES: CN(c1ccc(-c2ccc(-c3cn[nH]c3)c3cn[nH]c23)nn1)[C@@H]1C[C@H]2CCC[C@@H](C1)N2] |
| 296 | 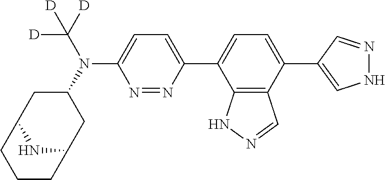 [SMILES: [2H]C([2H])([2H])N(c1ccc(-c2ccc(-c3cn[nH]c3)c3cn[nH]c23)nn1)[C@@H]1C[C@H]2CCC[C@@H](C1)N2] |
| 297 | 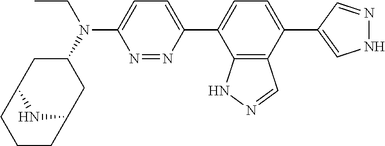 [SMILES: CCN(c1ccc(-c2ccc(-c3cn[nH]c3)c3cn[nH]c23)nn1)[C@@H]1C[C@H]2CCC[C@@H](C1)N2] |
| 298 | 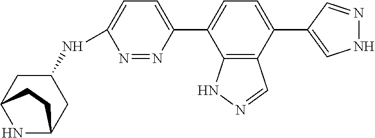 |
| 299 | 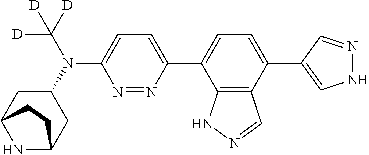 |
| 300 | 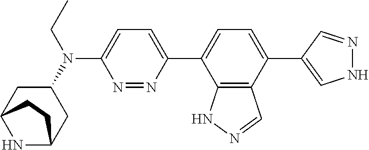 |
| 301 | 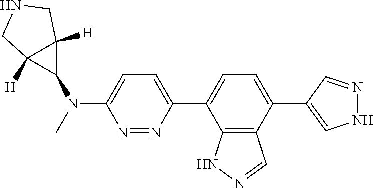 [SMILES: CN(c1ccc(-c2ccc(-c3cn[nH]c3)c3cn[nH]c23)nn1)[C@H]1[C@@H]2CNC[C@@H]21] |
| 302 | 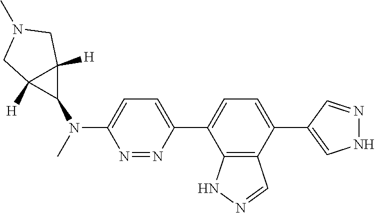 [SMILES: CN1C[C@@H]2[C@H](C1)[C@H]2N(C)c1ccc(-c2ccc(-c3cn[nH]c3)c3cn[nH]c23)nn1] |
| 303 | 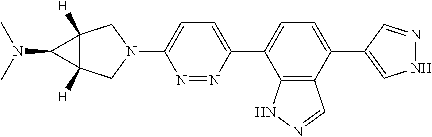 [SMILES: CN(C)[C@H]1[C@@H]2CN(c3ccc(-c4ccc(-c5cn[nH]c5)c5cn[nH]c45)nn3)C[C@@H]21] |
| 304 | 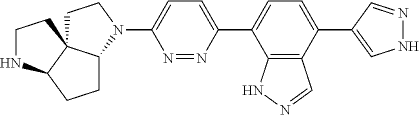 [SMILES: c1n[nH]cc1-c1ccc(-c2ccc(N3CC[C@@]45CCN[C@@H]4CC[C@@H]35)nn2)c2[nH]ncc12] |
| 305 | 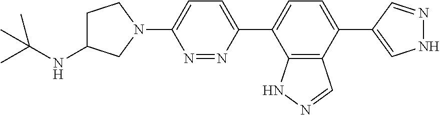 [SMILES: CC(C)(C)NC1CCN(c2ccc(-c3ccc(-c4cn[nH]c4)c4cn[nH]c34)nn2)C1] |
| 306 | 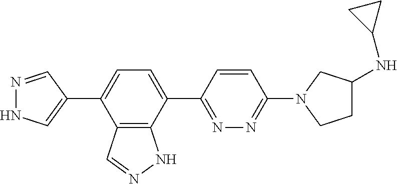 [SMILES: c1n[nH]cc1-c1ccc(-c2ccc(N3CCC(NC4CC4)C3)nn2)c2[nH]ncc12] |
| 307 | 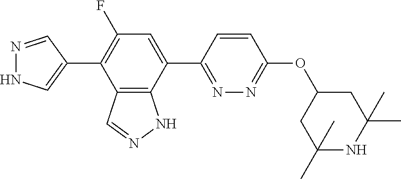 |
| 308 | 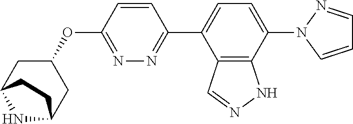 |
| 309 | 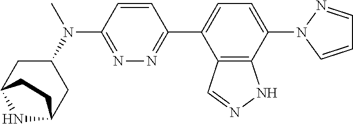 |
| 310 | 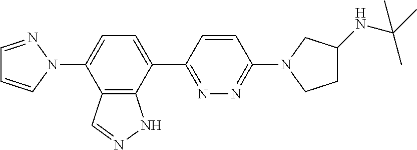 [SMILES: CC(C)(C)NC1CCN(c2ccc(-c3ccc(-n4cccn4)c4cn[nH]c34)nn2)C1] |
| 311 | 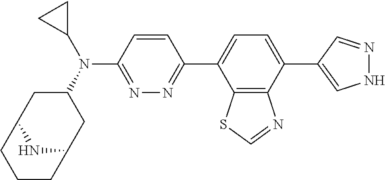 |
| 312 | 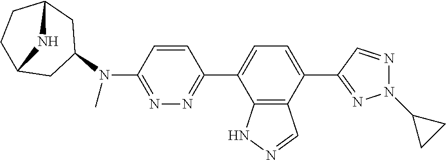 [SMILES: CN(c1ccc(-c2ccc(-c3cnn(C4CC4)n3)c3cn[nH]c23)nn1)[C@@H]1C[C@H]2CC[C@@H](C1)N2] |
| 313 | 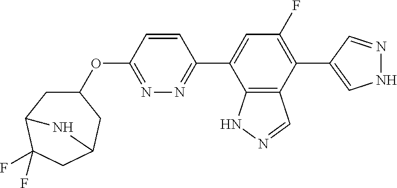 [SMILES: Fc1cc(-c2ccc(OC3CC4CC(F)(F)C(C3)N4)nn2)c2[nH]ncc2c1-c1cn[nH]c1] |
| 314 | 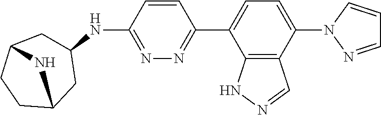 [SMILES: c1cnn(-c2ccc(-c3ccc(N[C@@H]4C[C@H]5CC[C@@H](C4)N5)nn3)c3[nH]ncc23)c1] |
| 315 | 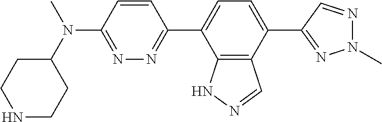 [SMILES: CN(c1ccc(-c2ccc(-c3cnn(C)n3)c3cn[nH]c23)nn1)C1CCNCC1] |
| 316 | 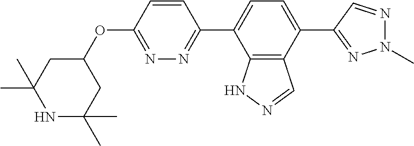 |
| 317 | 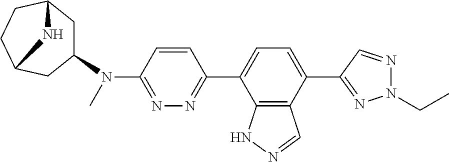 [SMILES: CCn1ncc(-c2ccc(-c3ccc(N(C)[C@@H]4C[C@H]5CC[C@@H](C4)N5)nn3)c3[nH]ncc23)n1] |
| 318 | 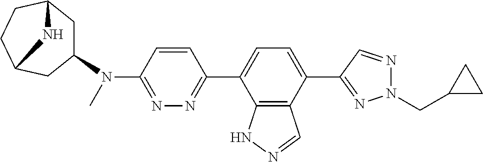 [SMILES: CN(c1ccc(-c2ccc(-c3cnn(CC4CC4)n3)c3cn[nH]c23)nn1)[C@@H]1C[C@H]2CC[C@@H](C1)N2] |
| 319 | 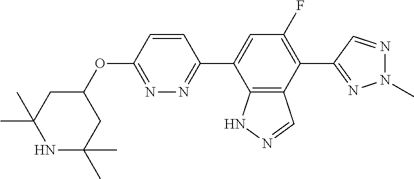 |
| 320 | 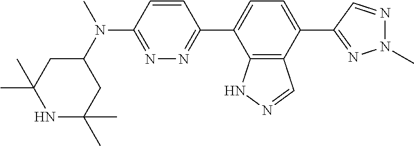 |
| 321 | 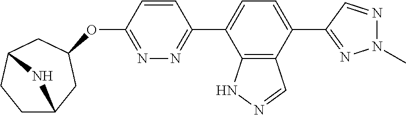 [SMILES: Cn1ncc(-c2ccc(-c3ccc(O[C@@H]4C[C@H]5CC[C@@H](C4)N5)nn3)c3[nH]ncc23)n1] |
| 322 | 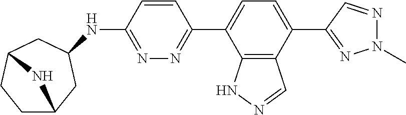 [SMILES: Cn1ncc(-c2ccc(-c3ccc(N[C@@H]4C[C@H]5CC[C@@H](C4)N5)nn3)c3[nH]ncc23)n1] |
| 323 | 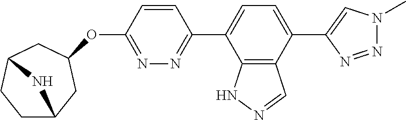 [SMILES: Cn1cc(-c2ccc(-c3ccc(O[C@@H]4C[C@H]5CC[C@@H](C4)N5)nn3)c3[nH]ncc23)nn1] |
| 324 | 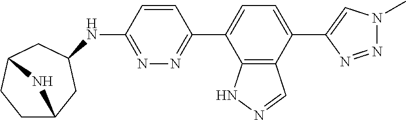 [SMILES: Cn1cc(-c2ccc(-c3ccc(N[C@@H]4C[C@H]5CC[C@@H](C4)N5)nn3)c3[nH]ncc23)nn1] |
| 325 | 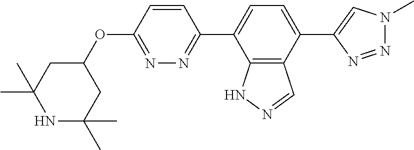 |
| 326 | 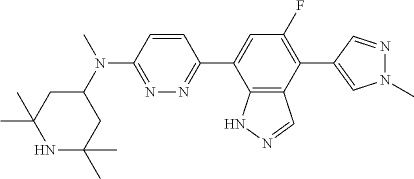 |
| 327 | 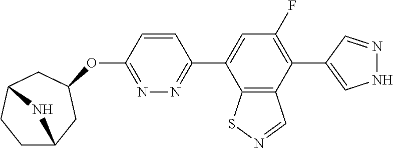 [SMILES: Fc1cc(-c2ccc(O[C@@H]3C[C@H]4CC[C@@H](C3)N4)nn2)c2sncc2c1-c1cn[nH]c1] |
| 328 | 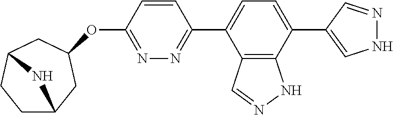 [SMILES: c1n[nH]cc1-c1ccc(-c2ccc(O[C@@H]3C[C@H]4CC[C@@H](C3)N4)nn2)c2cn[nH]c12] |
| 329 | 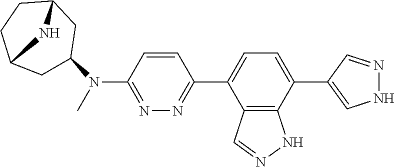 [SMILES: CN(c1ccc(-c2ccc(-c3cn[nH]c3)c3[nH]ncc23)nn1)[C@@H]1C[C@H]2CC[C@@H](C1)N2] |
| 330 | 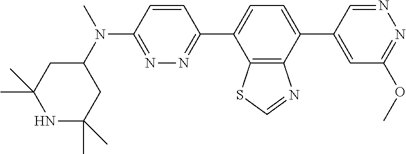 [SMILES: COc1cc(-c2ccc(-c3ccc(N(C)C4CC(C)(C)NC(C)(C)C4)nn3)c3scnc23)cnn1] |
| 331 | 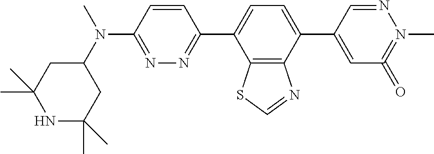 |
| 332 | 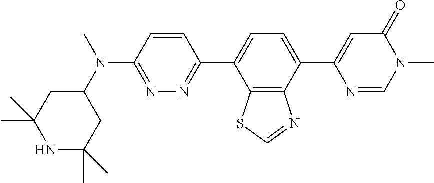 |
| 333 | 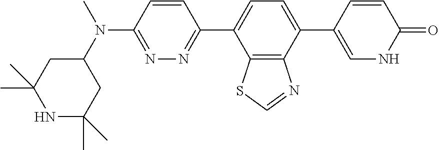 [SMILES: CN(c1ccc(-c2ccc(-c3ccc(=O)[nH]c3)c3ncsc23)nn1)C1CC(C)(C)NC(C)(C)C1] |
| 334 | 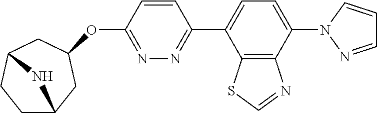 [SMILES: c1cnn(-c2ccc(-c3ccc(O[C@@H]4C[C@H]5CC[C@@H](C4)N5)nn3)c3scnc23)c1] |
| 335 | 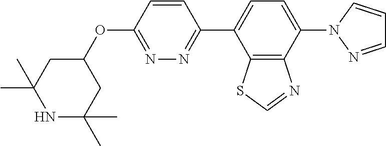 [SMILES: CC1(C)CC(Oc2ccc(-c3ccc(-n4cccn4)c4ncsc34)nn2)CC(C)(C)N1] |
| 336 | 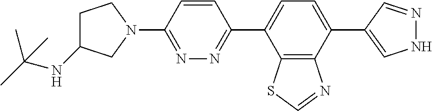 [SMILES: CC(C)(C)NC1CCN(c2ccc(-c3ccc(-c4cn[nH]c4)c4ncsc34)nn2)C1] |
| 337 | 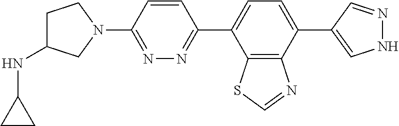 [SMILES: c1nc2c(-c3cn[nH]c3)ccc(-c3ccc(N4CCC(NC5CC5)C4)nn3)c2s1] |
| 338 | 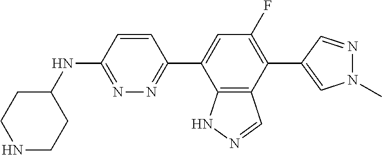 [SMILES: Cn1cc(-c2c(F)cc(-c3ccc(NC4CCNCC4)nn3)c3[nH]ncc23)cn1] |
| 339 | 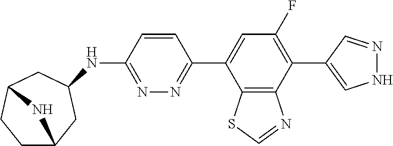 [SMILES: Fc1cc(-c2ccc(N[C@@H]3C[C@H]4CC[C@@H](C3)N4)nn2)c2scnc2c1-c1cn[nH]c1] |
| 340 | 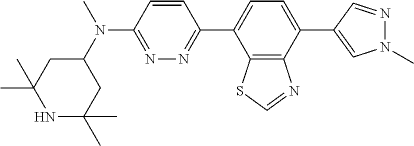 |
| 341 | 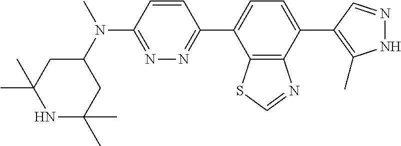 [SMILES: Cc1[nH]ncc1-c1ccc(-c2ccc(N(C)C3CC(C)(C)NC(C)(C)C3)nn2)c2scnc12] |
| 342 | 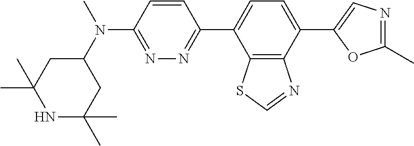 |
| 343 | 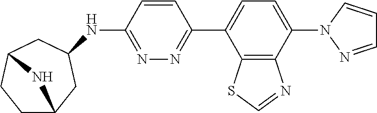 [SMILES: c1cnn(-c2ccc(-c3ccc(N[C@@H]4C[C@H]5CC[C@@H](C4)N5)nn3)c3scnc23)c1] |
| 344 | 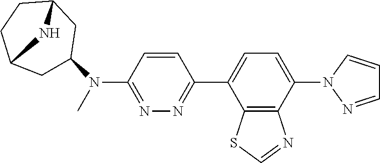 [SMILES: CN(c1ccc(-c2ccc(-n3cccn3)c3ncsc23)nn1)[C@@H]1C[C@H]2CC[C@@H](C1)N2] |
| 345 | 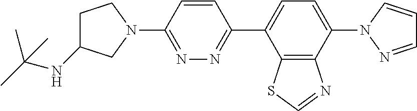 [SMILES: CC(C)(C)NC1CCN(c2ccc(-c3ccc(-n4cccn4)c4ncsc34)nn2)C1] |
| 346 | 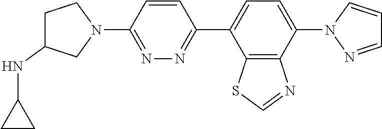 [SMILES: c1cnn(-c2ccc(-c3ccc(N4CCC(NC5CC5)C4)nn3)c3scnc23)c1] |
| 347 | 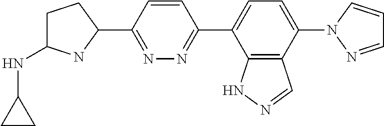 [SMILES: c1cnn(-c2ccc(-c3ccc(C4CCC(NC5CC5)N4)nn3)c3[nH]ncc23)c1] |
| 348 | 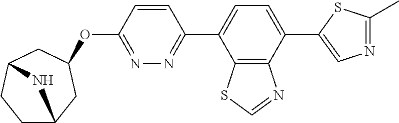 [SMILES: Cc1ncc(-c2ccc(-c3ccc(O[C@@H]4C[C@H]5CC[C@@H](C4)N5)nn3)c3scnc23)s1] |
| 349 | 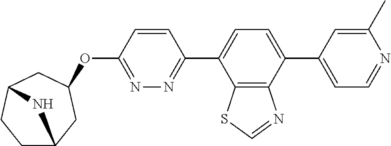 [SMILES: Cc1cc(-c2ccc(-c3ccc(O[C@@H]4C[C@H]5CC[C@@H](C4)N5)nn3)c3scnc23)ccn1] |
| 350 | 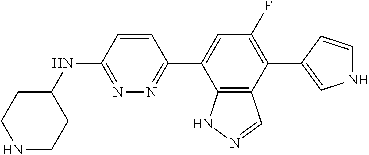 [SMILES: Fc1cc(-c2ccc(NC3CCNCC3)nn2)c2[nH]ncc2c1-c1cc[nH]c1] |
| 351 | 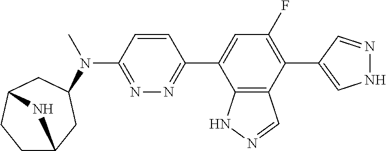 [SMILES: CN(c1ccc(-c2cc(F)c(-c3cn[nH]c3)c3cn[nH]c23)nn1)[C@@H]1C[C@H]2CC[C@@H](C1)N2] |
| 352 | 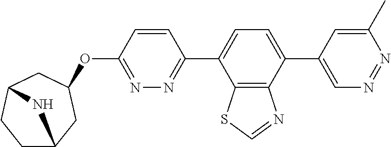 [SMILES: Cc1cc(-c2ccc(-c3ccc(O[C@@H]4C[C@H]5CC[C@@H](C4)N5)nn3)c3scnc23)cnn1] |
| 353 | 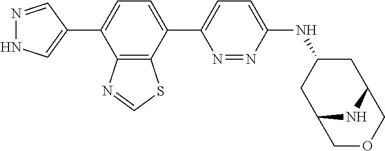 [SMILES: c1nc2c(-c3cn[nH]c3)ccc(-c3ccc(N[C@H]4C[C@H]5COC[C@@H](C4)N5)nn3)c2s1] |
| 354 | 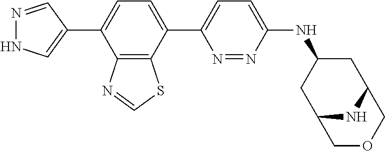 [SMILES: c1nc2c(-c3cn[nH]c3)ccc(-c3ccc(N[C@@H]4C[C@H]5COC[C@@H](C4)N5)nn3)c2s1] |
| 355 | 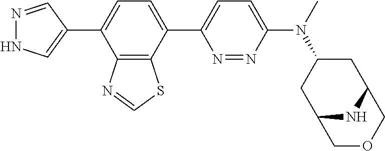 [SMILES: CN(c1ccc(-c2ccc(-c3cn[nH]c3)c3ncsc23)nn1)[C@H]1C[C@H]2COC[C@@H](C1)N2] |
| 356 | 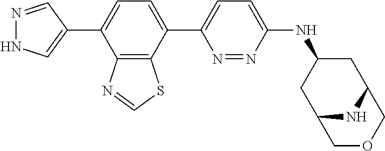 [SMILES: c1nc2c(-c3cn[nH]c3)ccc(-c3ccc(N[C@@H]4C[C@H]5COC[C@@H](C4)N5)nn3)c2s1] |
| 357 | 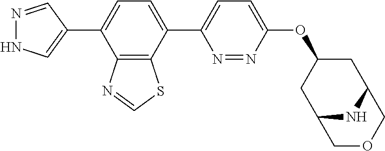 [SMILES: c1nc2c(-c3cn[nH]c3)ccc(-c3ccc(O[C@@H]4C[C@H]5COC[C@@H](C4)N5)nn3)c2s1] |
| 358 | 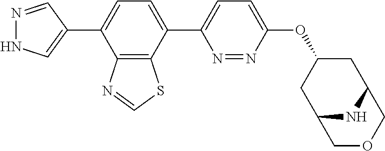 [SMILES: c1nc2c(-c3cn[nH]c3)ccc(-c3ccc(O[C@H]4C[C@H]5COC[C@@H](C4)N5)nn3)c2s1] |
| 359 | 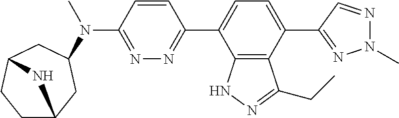 [SMILES: CCc1n[nH]c2c(-c3ccc(N(C)[C@@H]4C[C@H]5CC[C@@H](C4)N5)nn3)ccc(-c3cnn(C)n3)c12] |
| 360 | 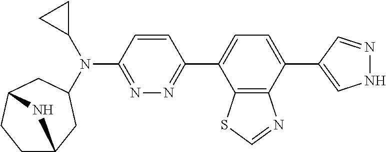 [SMILES: c1nc2c(-c3cn[nH]c3)ccc(-c3ccc(N(C4CC4)C4C[C@H]5CC[C@@H](C4)N5)nn3)c2s1] |
| 361 | 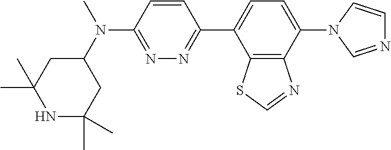 [SMILES: CN(c1ccc(-c2ccc(-n3ccnc3)c3ncsc23)nn1)C1CC(C)(C)NC(C)(C)C1] |
| 362 | 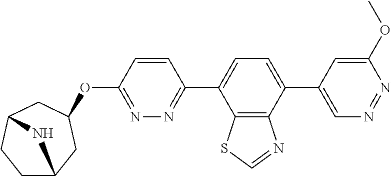 [SMILES: COc1cc(-c2ccc(-c3ccc(O[C@@H]4C[C@H]5CC[C@@H](C4)N5)nn3)c3scnc23)cnn1] |
| 363 | 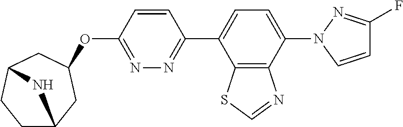 [SMILES: Fc1ccn(-c2ccc(-c3ccc(O[C@@H]4C[C@H]5CC[C@@H](C4)N5)nn3)c3scnc23)n1] |
| 364 | 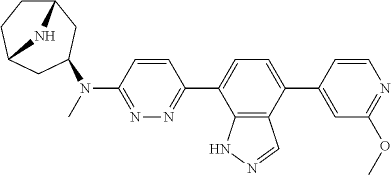 [SMILES: COc1cc(-c2ccc(-c3ccc(N(C)[C@@H]4C[C@H]5CC[C@@H](C4)N5)nn3)c3[nH]ncc23)ccn1] |
| 365 | 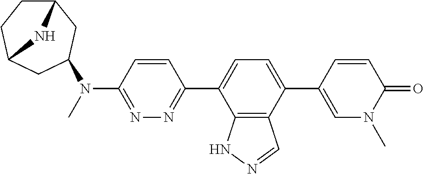 [SMILES: CN(c1ccc(-c2ccc(-c3ccc(=O)n(C)c3)c3cn[nH]c23)nn1)[C@@H]1C[C@H]2CC[C@@H](C1)N2] |
| 368 | 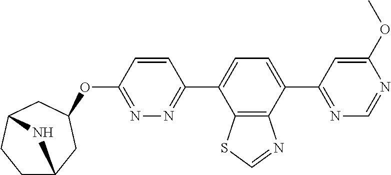 [SMILES: COc1cc(-c2ccc(-c3ccc(O[C@@H]4C[C@H]5CC[C@@H](C4)N5)nn3)c3scnc23)ncn1] |
| 369 | 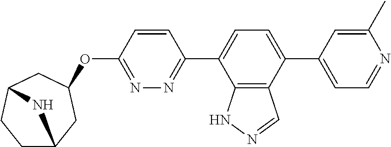 [SMILES: Cc1cc(-c2ccc(-c3ccc(O[C@@H]4C[C@H]5CC[C@@H](C4)N5)nn3)c3[nH]ncc23)ccn1] |
| 370 | 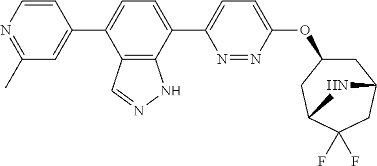 [SMILES: Cc1cc(-c2ccc(-c3ccc(O[C@H]4C[C@@H]5CC(F)(F)[C@H](C4)N5)nn3)c3[nH]ncc23)ccn1] |
| 371 | 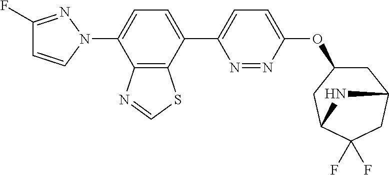 [SMILES: Fc1ccn(-c2ccc(-c3ccc(O[C@H]4C[C@@H]5CC(F)(F)[C@H](C4)N5)nn3)c3scnc23)n1] |
| 373 | 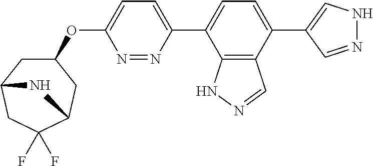 [SMILES: FC1(F)C[C@@H]2C[C@@H](Oc3ccc(-c4ccc(-c5cn[nH]c5)c5cn[nH]c45)nn3)C[C@H]1N2] |
| 374 | 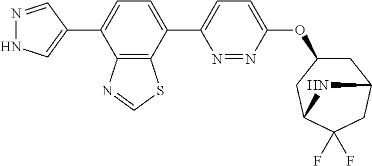 [SMILES: FC1(F)C[C@H]2C[C@H](Oc3ccc(-c4ccc(-c5cn[nH]c5)c5ncsc45)nn3)C[C@@H]1N2] |
| 375 | 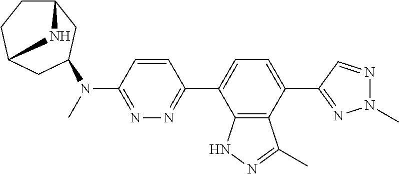 [SMILES: Cc1n[nH]c2c(-c3ccc(N(C)[C@@H]4C[C@H]5CC[C@@H](C4)N5)nn3)ccc(-c3cnn(C)n3)c12] |
| 376 | 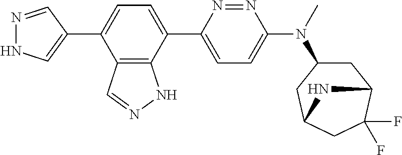 [SMILES: CN(c1ccc(-c2ccc(-c3cn[nH]c3)c3cn[nH]c23)nn1)[C@@H]1C[C@H]2CC(F)(F)[C@@H](C1)N2] |
| 377 | 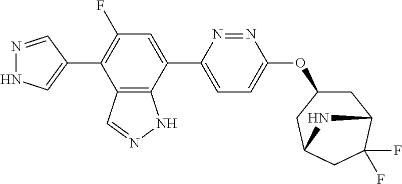 [SMILES: Fc1cc(-c2ccc(O[C@@H]3C[C@H]4CC(F)(F)[C@@H](C3)N4)nn2)c2[nH]ncc2c1-c1cn[nH]c1] |
| 378 | 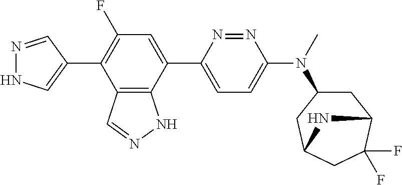 [SMILES: CN(c1ccc(-c2cc(F)c(-c3cn[nH]c3)c3cn[nH]c23)nn1)[C@@H]1C[C@H]2CC(F)(F)[C@@H](C1)N2] |
| 379 | 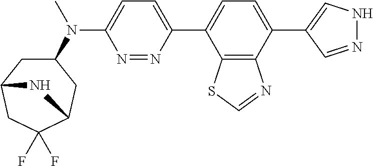 [SMILES: CN(c1ccc(-c2ccc(-c3cn[nH]c3)c3ncsc23)nn1)[C@@H]1C[C@H]2CC(F)(F)[C@@H](C1)N2] |
| 380 | 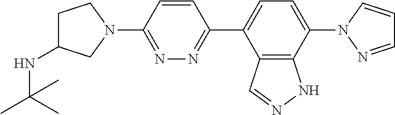 [SMILES: CC(C)(C)NC1CCN(c2ccc(-c3ccc(-n4cccn4)c4[nH]ncc34)nn2)C1] |
| 381 | 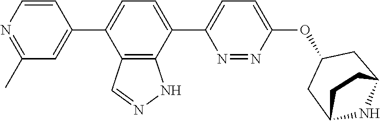 |
| 382 | 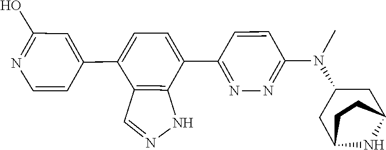 |
| 383 | 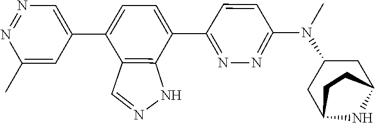 |
| 385 | 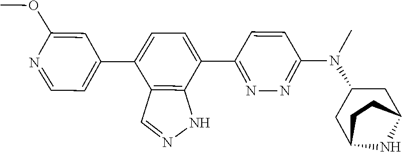 |
| 386 | 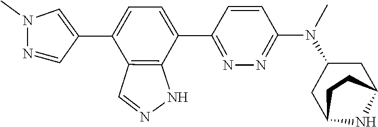 |
| 390 | 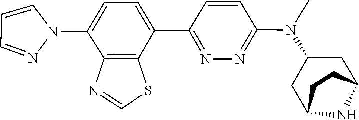 |
| 391 | 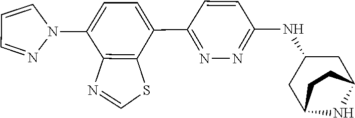 |
| 392 | 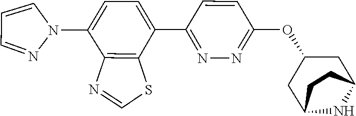 |
| 393 | 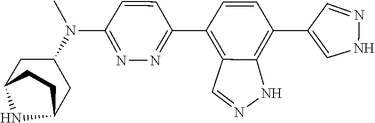 |
| 394 | 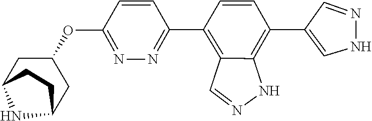 |
| 395 | 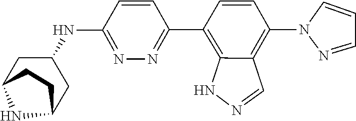 |
| 396 | 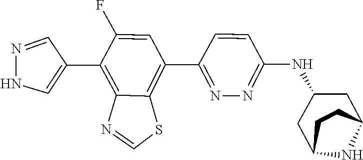 |
| 397 | 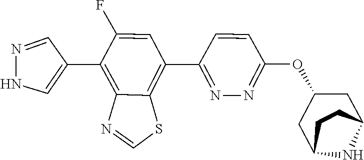 |
| 402 | 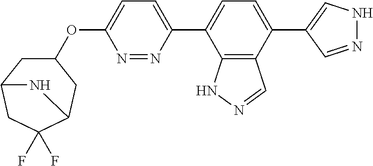 [SMILES: FC1(F)CC2CC(Oc3ccc(-c4ccc(-c5cn[nH]c5)c5cn[nH]c45)nn3)CC1N2] |
| 404 | 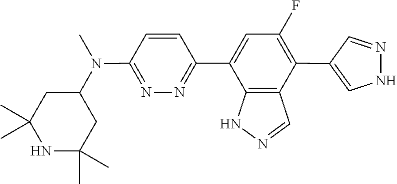 [SMILES: CN(c1ccc(-c2cc(F)c(-c3cn[nH]c3)c3cn[nH]c23)nn1)C1CC(C)(C)NC(C)(C)C1] |
| 405 | 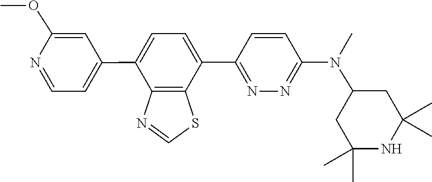 |
| 406 | 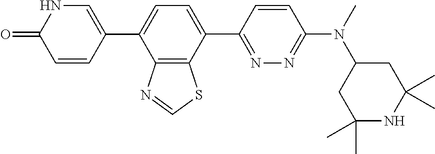 [SMILES: CN(c1ccc(-c2ccc(-c3ccc(=O)[nH]c3)c3ncsc23)nn1)C1CC(C)(C)NC(C)(C)C1] |
| 407 | 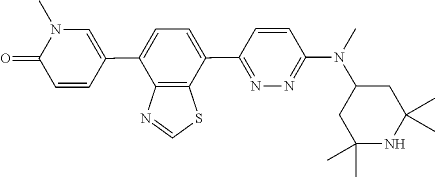 [SMILES: CN(c1ccc(-c2ccc(-c3ccc(=O)n(C)c3)c3ncsc23)nn1)C1CC(C)(C)NC(C)(C)C1] |
| 408 | 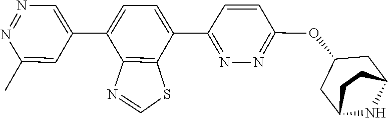 |
| 409 | 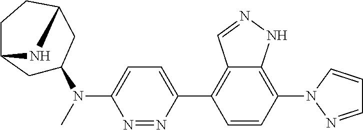 [SMILES: CN(c1ccc(-c2ccc(-n3cccn3)c3[nH]ncc23)nn1)[C@@H]1C[C@H]2CC[C@@H](C1)N2] |
| 410 | 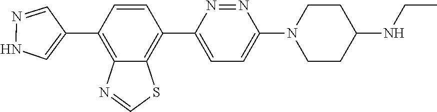 [SMILES: CCNC1CCN(c2ccc(-c3ccc(-c4cn[nH]c4)c4ncsc34)nn2)CC1] |
| 411 | 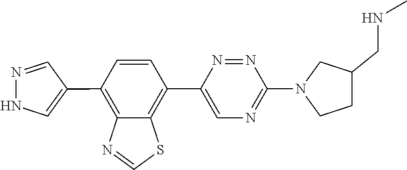 [SMILES: CNCC1CCN(c2ncc(-c3ccc(-c4cn[nH]c4)c4ncsc34)nn2)C1] |
| 412 | 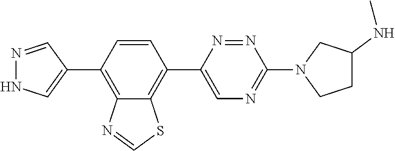 [SMILES: CNC1CCN(c2ncc(-c3ccc(-c4cn[nH]c4)c4ncsc34)nn2)C1] |
| 413 | 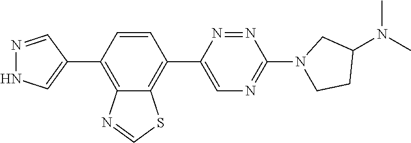 [SMILES: CN(C)C1CCN(c2ncc(-c3ccc(-c4cn[nH]c4)c4ncsc34)nn2)C1] |
| 414 | 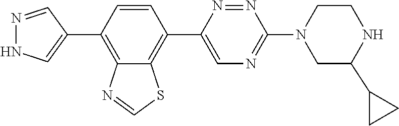 [SMILES: c1nc2c(-c3cn[nH]c3)ccc(-c3cnc(N4CCNC(C5CC5)C4)nn3)c2s1] |
| 415 | 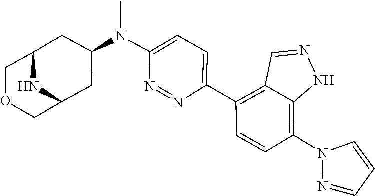 |
| 416 | 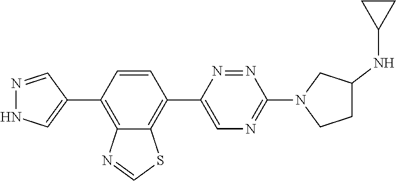 [SMILES: c1nc2c(-c3cn[nH]c3)ccc(-c3cnc(N4CCC(NC5CC5)C4)nn3)c2s1] |
| 417 | 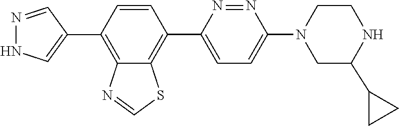 [SMILES: c1nc2c(-c3cn[nH]c3)ccc(-c3ccc(N4CCNC(C5CC5)C4)nn3)c2s1] |
| 418 | 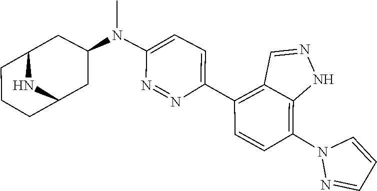 |
| 419 | 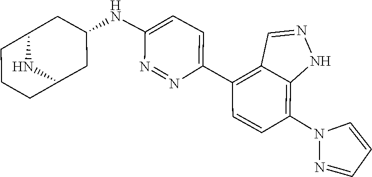 |
| 420 | 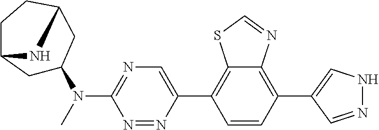 [SMILES: CN(c1ncc(-c2ccc(-c3cn[nH]c3)c3ncsc23)nn1)[C@@H]1C[C@H]2CC[C@@H](C1)N2] |
| 421 | 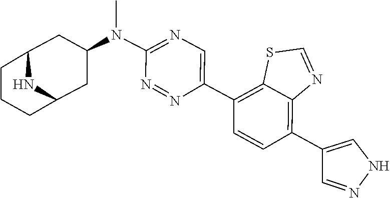 |
| 422 | 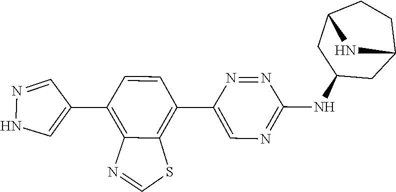 [SMILES: c1nc2c(-c3cn[nH]c3)ccc(-c3cnc(N[C@@H]4C[C@H]5CC[C@@H](C4)N5)nn3)c2s1] |
| 423 | 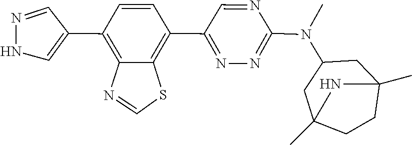 [SMILES: CN(c1ncc(-c2ccc(-c3cn[nH]c3)c3ncsc23)nn1)C1CC2(C)CCC(C)(C1)N2] |
| 424 | 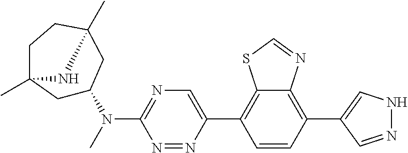 [SMILES: CN(c1ncc(-c2ccc(-c3cn[nH]c3)c3ncsc23)nn1)[C@H]1C[C@]2(C)CC[C@](C)(C1)N2] |
| 425 | 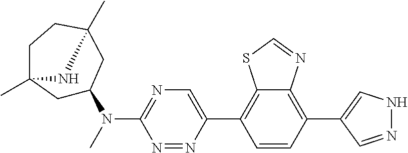 [SMILES: CN(c1ncc(-c2ccc(-c3cn[nH]c3)c3ncsc23)nn1)[C@@H]1C[C@]2(C)CC[C@](C)(C1)N2] |
| 426 | 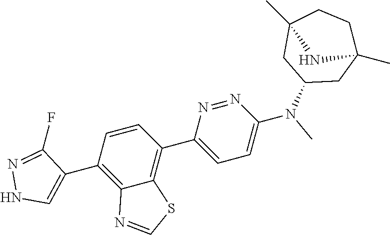 [SMILES: CN(c1ccc(-c2ccc(-c3c[nH]nc3F)c3ncsc23)nn1)[C@H]1C[C@]2(C)CC[C@](C)(C1)N2] |
| 427 | 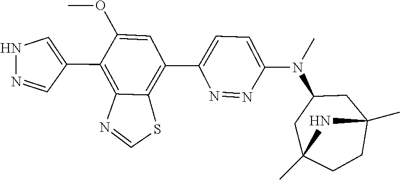 [SMILES: COc1cc(-c2ccc(N(C)[C@H]3C[C@]4(C)CC[C@](C)(C3)N4)nn2)c2scnc2c1-c1cn[nH]c1] |
| 428 | 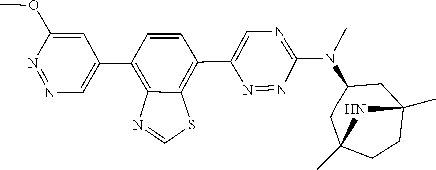 |
| 429 | 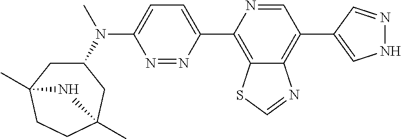 [SMILES: CN(c1ccc(-c2ncc(-c3cn[nH]c3)c3ncsc23)nn1)[C@H]1C[C@]2(C)CC[C@](C)(C1)N2] |
| 430 | 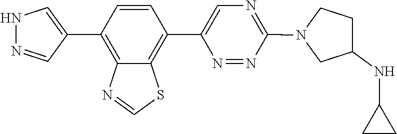 [SMILES: c1nc2c(-c3cn[nH]c3)ccc(-c3cnc(N4CCC(NC5CC5)C4)nn3)c2s1] |
| 431 | 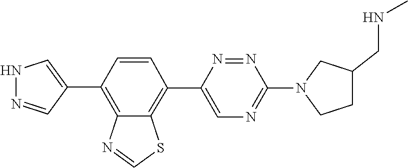 [SMILES: CNCC1CCN(c2ncc(-c3ccc(-c4cn[nH]c4)c4ncsc34)nn2)C1] |
| 432 | 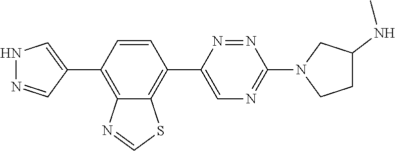 [SMILES: CNC1CCN(c2ncc(-c3ccc(-c4cn[nH]c4)c4ncsc34)nn2)C1] |
| 433 | 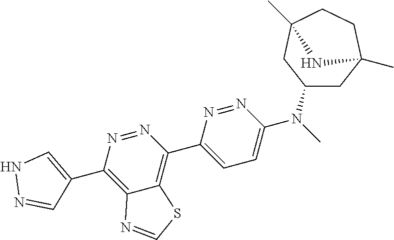 [SMILES: CN(c1ccc(-c2nnc(-c3cn[nH]c3)c3ncsc23)nn1)[C@@H]1CC[C@@]2(C)CC[C@@](C)(C1)N2] |
| 434 | 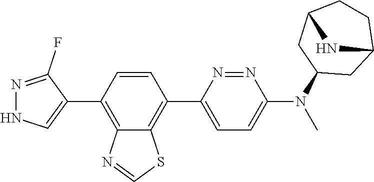 [SMILES: CN(c1ccc(-c2ccc(-c3c[nH]nc3F)c3ncsc23)nn1)[C@@H]1C[C@H]2CC[C@@H](C1)N2] |
| 435 | 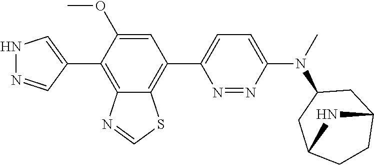 [SMILES: COc1cc(-c2ccc(N(C)[C@@H]3C[C@H]4CC[C@@H](C3)N4)nn2)c2scnc2c1-c1cn[nH]c1] |
| 436 | 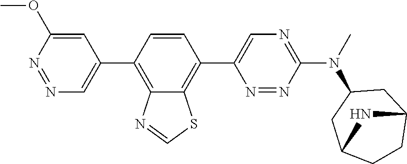 [SMILES: COc1cc(-c2ccc(-c3cnc(N(C)[C@@H]4C[C@H]5CC[C@@H](C4)N5)nn3)c3scnc23)cnn1] |
| 437 | 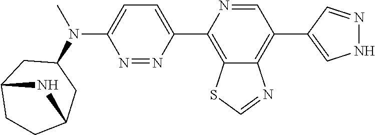 [SMILES: CN(c1ccc(-c2ncc(-c3cn[nH]c3)c3ncsc23)nn1)[C@@H]1C[C@H]2CC[C@@H](C1)N2] |
| 438 | 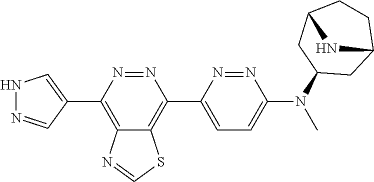 [SMILES: CN(c1ccc(-c2nnc(-c3cn[nH]c3)c3ncsc23)nn1)[C@@H]1C[C@H]2CC[C@@H](C1)N2] |
| 439 | 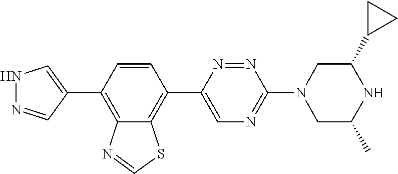 [SMILES: C[C@@H]1CN(c2ncc(-c3ccc(-c4cn[nH]c4)c4ncsc34)nn2)C[C@H](C2CC2)N1] |
| 440 | 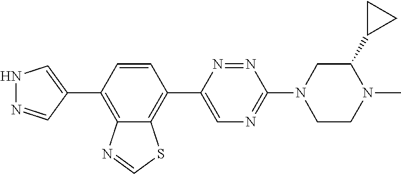 [SMILES: CN1CCN(c2ncc(-c3ccc(-c4cn[nH]c4)c4ncsc34)nn2)C[C@@H]1C1CC1] |
| 441 | 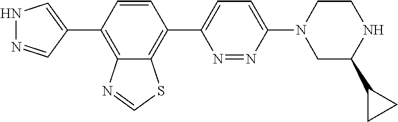 [SMILES: c1nc2c(-c3cn[nH]c3)ccc(-c3ccc(N4CCN[C@@H](C5CC5)C4)nn3)c2s1] |
| 442 | 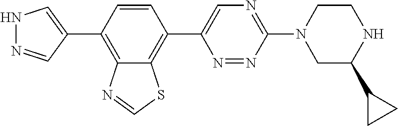 [SMILES: c1nc2c(-c3cn[nH]c3)ccc(-c3cnc(N4CCN[C@@H](C5CC5)C4)nn3)c2s1] |
| 443 | 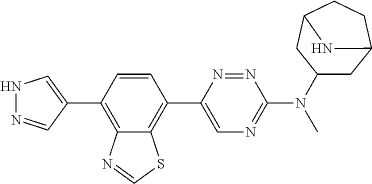 [SMILES: CN(c1ncc(-c2ccc(-c3cn[nH]c3)c3ncsc23)nn1)C1CC2CCC(C1)N2] |
| 444 | 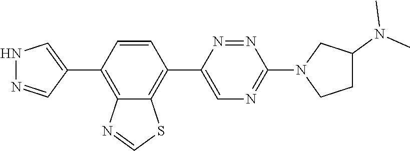 [SMILES: CN(C)C1CCN(c2ncc(-c3ccc(-c4cn[nH]c4)c4ncsc34)nn2)C1] |
| 445 | 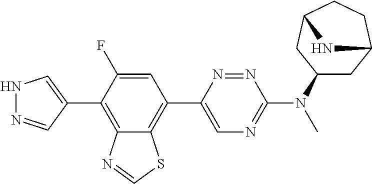 [SMILES: CN(c1ncc(-c2cc(F)c(-c3cn[nH]c3)c3ncsc23)nn1)[C@@H]1C[C@H]2CC[C@@H](C1)N2] |
| 446 | 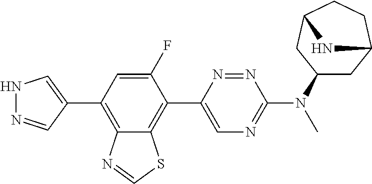 [SMILES: CN(c1ncc(-c2c(F)cc(-c3cn[nH]c3)c3ncsc23)nn1)[C@@H]1C[C@H]2CC[C@@H](C1)N2] |
| 447 | 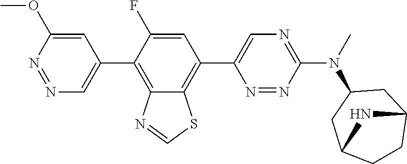 [SMILES: COc1cc(-c2c(F)cc(-c3cnc(N(C)[C@@H]4C[C@H]5CC[C@@H](C4)N5)nn3)c3scnc23)cnn1] |
| 448 | 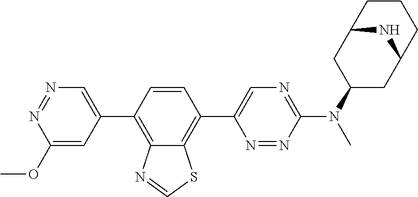 [SMILES: COc1cc(-c2ccc(-c3cnc(N(C)[C@@H]4C[C@H]5CCC[C@@H](C4)N5)nn3)c3scnc23)cnn1] |
| 449 | 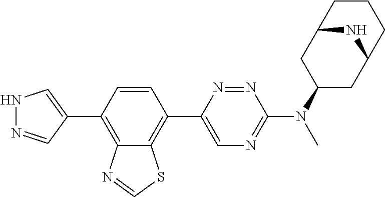 [SMILES: CN(c1ncc(-c2ccc(-c3cn[nH]c3)c3ncsc23)nn1)[C@@H]1C[C@H]2CCC[C@@H](C1)N2] |
| 450 | 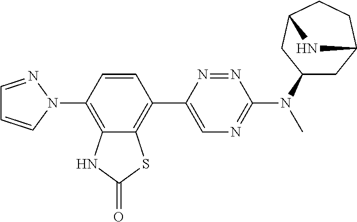 [SMILES: CN(c1ncc(-c2ccc(-n3cccn3)c3[nH]c(=O)sc23)nn1)[C@@H]1C[C@H]2CC[C@@H](C1)N2] |
| 451 | 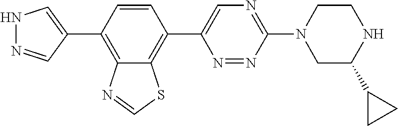 [SMILES: c1nc2c(-c3cn[nH]c3)ccc(-c3cnc(N4CCN[C@H](C5CC5)C4)nn3)c2s1] |
| 452 | 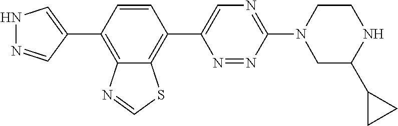 [SMILES: c1nc2c(-c3cn[nH]c3)ccc(-c3cnc(N4CCNC(C5CC5)C4)nn3)c2s1] |
| 453 | 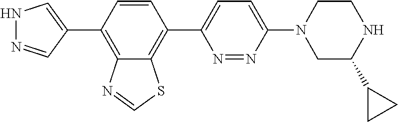 [SMILES: c1nc2c(-c3cn[nH]c3)ccc(-c3ccc(N4CCN[C@H](C5CC5)C4)nn3)c2s1] |
| 454 | 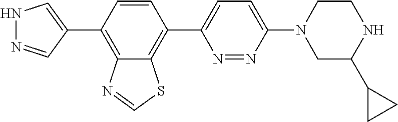 [SMILES: c1nc2c(-c3cn[nH]c3)ccc(-c3ccc(N4CCNC(C5CC5)C4)nn3)c2s1] |
| 455 | 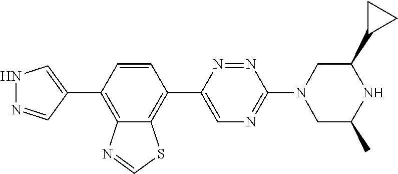 [SMILES: C[C@H]1CN(c2ncc(-c3ccc(-c4cn[nH]c4)c4ncsc34)nn2)C[C@@H](C2CC2)N1] |
| 456 | 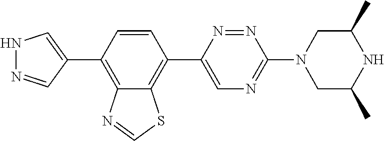 [SMILES: C[C@@H]1CN(c2ncc(-c3ccc(-c4cn[nH]c4)c4ncsc34)nn2)C[C@H](C)N1] |
| 457 | 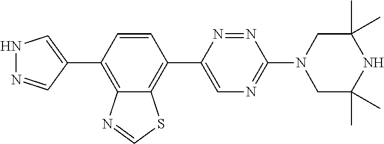 [SMILES: CC1(C)CN(c2ncc(-c3ccc(-c4cn[nH]c4)c4ncsc34)nn2)CC(C)(C)N1] |
| 458 | 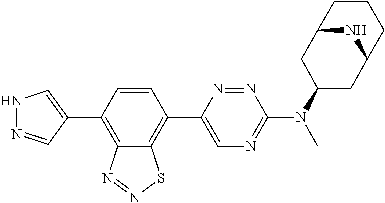 [SMILES: CN(c1ncc(-c2ccc(-c3cn[nH]c3)c3nnsc23)nn1)[C@@H]1C[C@H]2CCC[C@@H](C1)N2] |
| 459 | 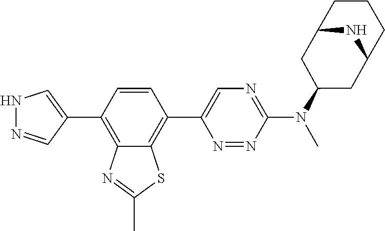 [SMILES: Cc1nc2c(-c3cn[nH]c3)ccc(-c3cnc(N(C)[C@@H]4C[C@H]5CCC[C@@H](C4)N5)nn3)c2s1] |
| 460 | 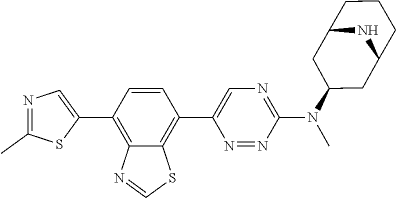 [SMILES: Cc1ncc(-c2ccc(-c3cnc(N(C)[C@@H]4C[C@H]5CCC[C@@H](C4)N5)nn3)c3scnc23)s1] |
| 461 | 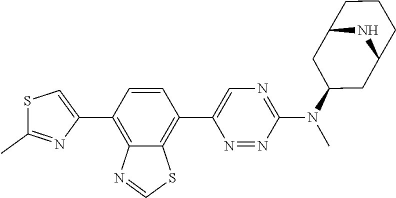 [SMILES: Cc1nc(-c2ccc(-c3cnc(N(C)[C@@H]4C[C@H]5CCC[C@@H](C4)N5)nn3)c3scnc23)cs1] |
| 462 | 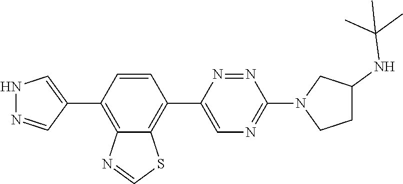 [SMILES: CC(C)(C)NC1CCN(c2ncc(-c3ccc(-c4cn[nH]c4)c4ncsc34)nn2)C1] |
| 463 | 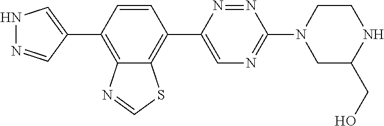 [SMILES: OCC1CN(c2ncc(-c3ccc(-c4cn[nH]c4)c4ncsc34)nn2)CCN1] |
| 464 | 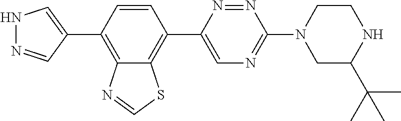 [SMILES: CC(C)(C)C1CN(c2ncc(-c3ccc(-c4cn[nH]c4)c4ncsc34)nn2)CCN1] |
| 465 | 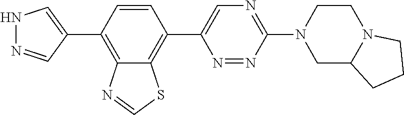 [SMILES: c1nc2c(-c3cn[nH]c3)ccc(-c3cnc(N4CCN5CCCC5C4)nn3)c2s1] |
| 466 | 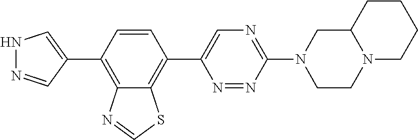 [SMILES: c1nc2c(-c3cn[nH]c3)ccc(-c3cnc(N4CCN5CCCCC5C4)nn3)c2s1] |
| 467 | 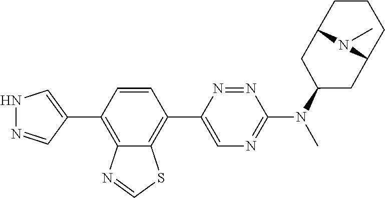 [SMILES: CN(c1ncc(-c2ccc(-c3cn[nH]c3)c3ncsc23)nn1)[C@@H]1C[C@H]2CCC[C@@H](C1)N2C] |
| 468 | 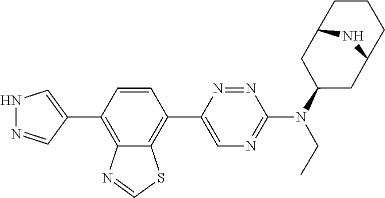 [SMILES: CCN(c1ncc(-c2ccc(-c3cn[nH]c3)c3ncsc23)nn1)[C@@H]1C[C@H]2CCC[C@@H](C1)N2] |
| 469 | 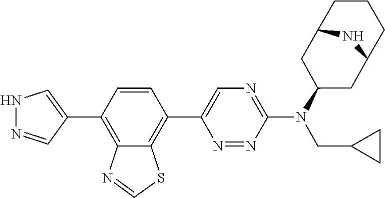 [SMILES: c1nc2c(-c3cn[nH]c3)ccc(-c3cnc(N(CC4CC4)[C@@H]4C[C@H]5CCC[C@@H](C4)N5)nn3)c2s1] |
| 470 | 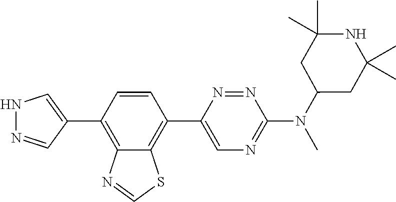 [SMILES: CN(c1ncc(-c2ccc(-c3cn[nH]c3)c3ncsc23)nn1)C1CC(C)(C)NC(C)(C)C1] |
| 471 | 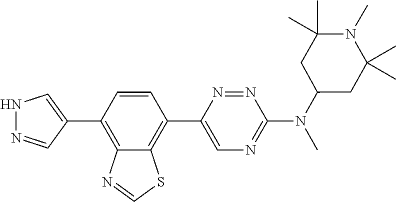 [SMILES: CN(c1ncc(-c2ccc(-c3cn[nH]c3)c3ncsc23)nn1)C1CC(C)(C)N(C)C(C)(C)C1] |
| 472 | 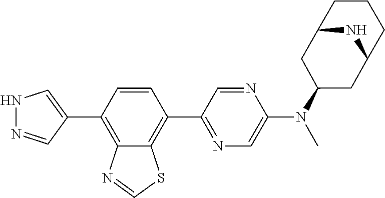 [SMILES: CN(c1cnc(-c2ccc(-c3cn[nH]c3)c3ncsc23)cn1)[C@@H]1C[C@H]2CCC[C@@H](C1)N2] |
| 473 | 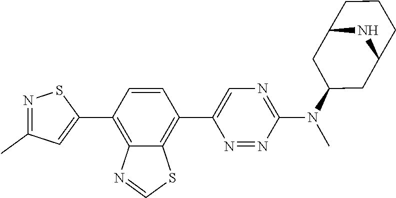 [SMILES: Cc1cc(-c2ccc(-c3cnc(N(C)[C@@H]4C[C@H]5CCC[C@@H](C4)N5)nn3)c3scnc23)sn1] |
| 474 | 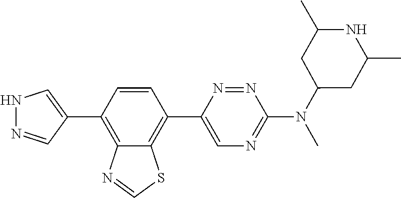 [SMILES: CC1CC(N(C)c2ncc(-c3ccc(-c4cn[nH]c4)c4ncsc34)nn2)CC(C)N1] |
| 475 | 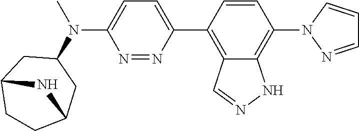 [SMILES: CN(c1ccc(-c2ccc(-n3cccn3)c3[nH]ncc23)nn1)[C@@H]1C[C@H]2CC[C@@H](C1)N2] |
| 476 | 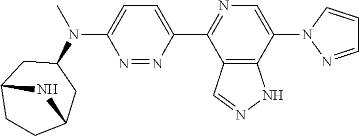 [SMILES: CN(c1ccc(-c2ncc(-n3cccn3)c3[nH]ncc23)nn1)[C@@H]1C[C@H]2CC[C@@H](C1)N2] |
| 477 | 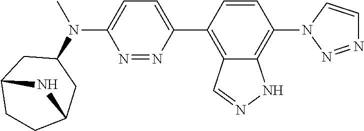 [SMILES: CN(c1ccc(-c2ccc(-n3ccnn3)c3[nH]ncc23)nn1)[C@@H]1C[C@H]2CC[C@@H](C1)N2] |
| 478 | 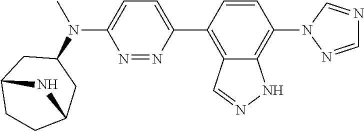 [SMILES: CN(c1ccc(-c2ccc(-n3cncn3)c3[nH]ncc23)nn1)[C@@H]1C[C@H]2CC[C@@H](C1)N2] |
| 479 | 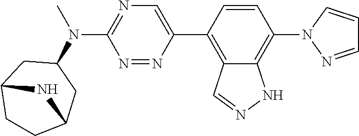 [SMILES: CN(c1ncc(-c2ccc(-n3cccn3)c3[nH]ncc23)nn1)[C@@H]1C[C@H]2CC[C@@H](C1)N2] |
| 480 | 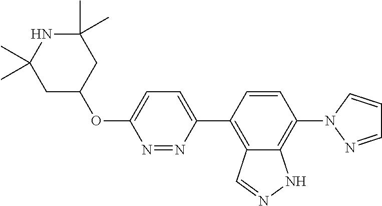 [SMILES: CC1(C)CC(Oc2ccc(-c3ccc(-n4cccn4)c4[nH]ncc34)nn2)CC(C)(C)N1] |
| 481 | 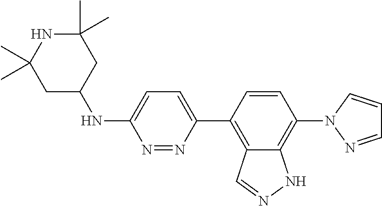 [SMILES: CC1(C)CC(Nc2ccc(-c3ccc(-n4cccn4)c4[nH]ncc34)nn2)CC(C)(C)N1] |
| 482 | 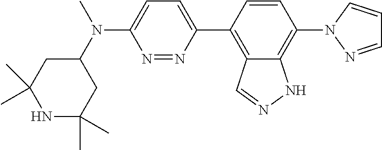 [SMILES: CN(c1ccc(-c2ccc(-n3cccn3)c3[nH]ncc23)nn1)C1CC(C)(C)NC(C)(C)C1] |
| 483 | 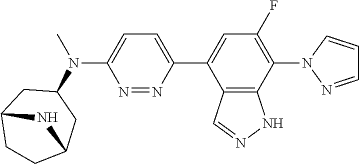 [SMILES: CN(c1ccc(-c2cc(F)c(-n3cccn3)c3[nH]ncc23)nn1)[C@@H]1C[C@H]2CC[C@@H](C1)N2] |
| 484 | 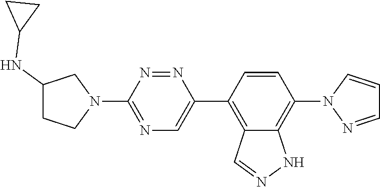 [SMILES: c1cnn(-c2ccc(-c3cnc(N4CCC(NC5CC5)C4)nn3)c3cn[nH]c23)c1] |
| 485 | 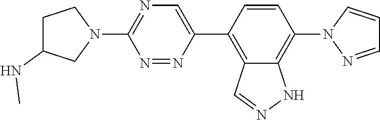 [SMILES: CNC1CCN(c2ncc(-c3ccc(-n4cccn4)c4[nH]ncc34)nn2)C1] |
| 486 | 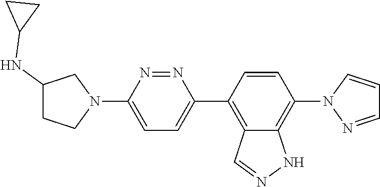 [SMILES: c1cnn(-c2ccc(-c3ccc(N4CCC(NC5CC5)C4)nn3)c3cn[nH]c23)c1] |
| 487 | 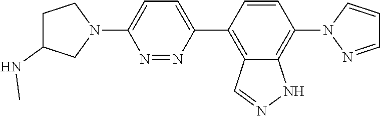 [SMILES: CNC1CCN(c2ccc(-c3ccc(-n4cccn4)c4[nH]ncc34)nn2)C1] |
| 488 | 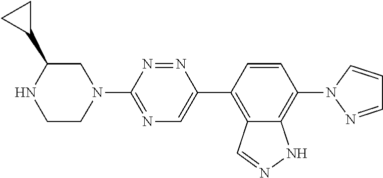 [SMILES: c1cnn(-c2ccc(-c3cnc(N4CCN[C@@H](C5CC5)C4)nn3)c3cn[nH]c23)c1] |
| 489 | 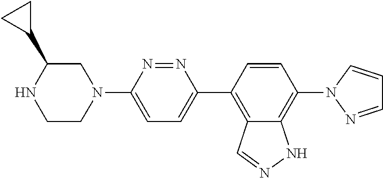 [SMILES: c1cnn(-c2ccc(-c3ccc(N4CCN[C@@H](C5CC5)C4)nn3)c3cn[nH]c23)c1] |
| 490 | 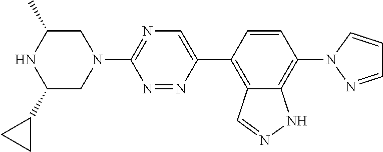 [SMILES: C[C@@H]1CN(c2ncc(-c3ccc(-n4cccn4)c4[nH]ncc34)nn2)C[C@H](C2CC2)N1] |
| 491 | 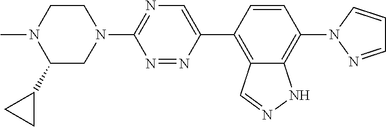 [SMILES: CN1CCN(c2ncc(-c3ccc(-n4cccn4)c4[nH]ncc34)nn2)C[C@@H]1C1CC1] |
| 492 | 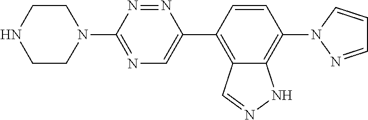 [SMILES: c1cnn(-c2ccc(-c3cnc(N4CCNCC4)nn3)c3cn[nH]c23)c1] |
| 493 | 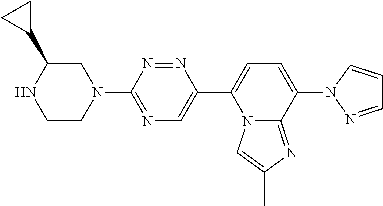 [SMILES: Cc1cn2c(-c3cnc(N4CCN[C@@H](C5CC5)C4)nn3)ccc(-n3cccn3)c2n1] |
| 494 | 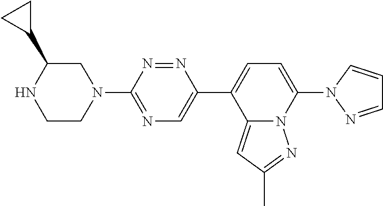 [SMILES: Cc1cc2c(-c3cnc(N4CCN[C@@H](C5CC5)C4)nn3)ccc(-n3cccn3)n2n1] |
| 495 | 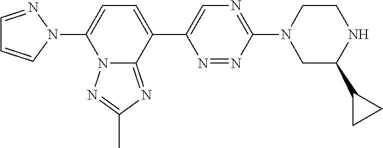 [SMILES: Cc1nc2c(-c3cnc(N4CCN[C@@H](C5CC5)C4)nn3)ccc(-n3cccn3)n2n1] |
| 496 | 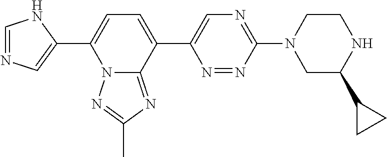 [SMILES: Cc1nc2c(-c3cnc(N4CCN[C@@H](C5CC5)C4)nn3)ccc(-c3cnc[nH]3)n2n1] |
| 497 | 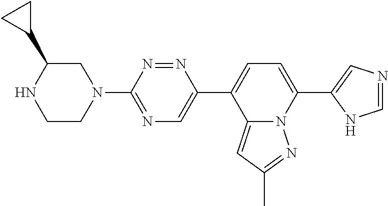 [SMILES: Cc1cc2c(-c3cnc(N4CCN[C@@H](C5CC5)C4)nn3)ccc(-c3cnc[nH]3)n2n1] |
| 498 | 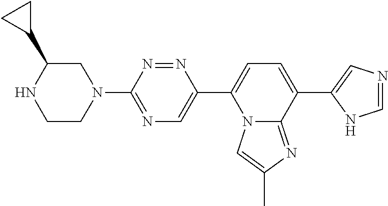 [SMILES: Cc1cn2c(-c3cnc(N4CCN[C@@H](C5CC5)C4)nn3)ccc(-c3cnc[nH]3)c2n1] |
| 499 | 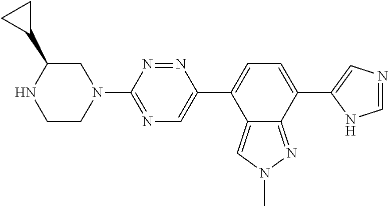 [SMILES: Cn1cc2c(-c3cnc(N4CCN[C@@H](C5CC5)C4)nn3)ccc(-c3cnc[nH]3)c2n1] |
| 500 | 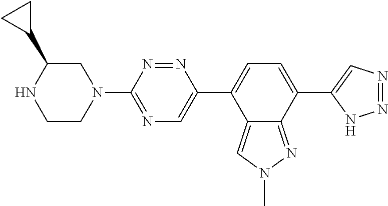 [SMILES: Cn1cc2c(-c3cnc(N4CCN[C@@H](C5CC5)C4)nn3)ccc(-c3cnn[nH]3)c2n1] |
| 501 | 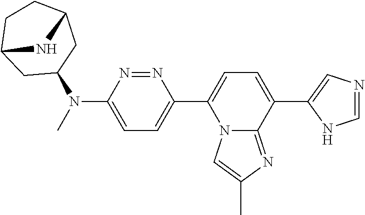 [SMILES: Cc1cn2c(-c3ccc(N(C)[C@@H]4C[C@H]5CC[C@@H](C4)N5)nn3)ccc(-c3cnc[nH]3)c2n1] |
| 502 | 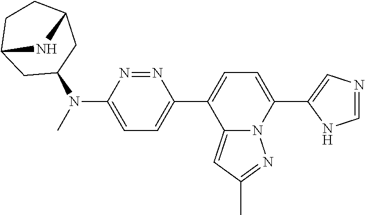 [SMILES: Cc1cc2c(-c3ccc(N(C)[C@@H]4C[C@H]5CC[C@@H](C4)N5)nn3)ccc(-c3cnc[nH]3)n2n1] |
| 503 | 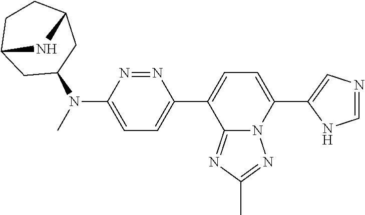 [SMILES: Cc1nc2c(-c3ccc(N(C)[C@@H]4C[C@H]5CC[C@@H](C4)N5)nn3)ccc(-c3cnc[nH]3)n2n1] |
| 504 | 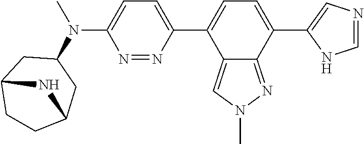 [SMILES: CN(c1ccc(-c2ccc(-c3cnc[nH]3)c3nn(C)cc23)nn1)[C@@H]1C[C@H]2CC[C@@H](C1)N2] |
| 505 | 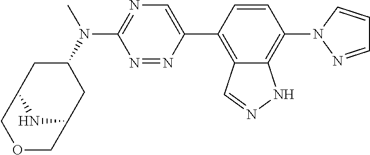 [SMILES: CN(c1ncc(-c2ccc(-n3cccn3)c3[nH]ncc23)nn1)[C@@H]1C[C@H]2COC[C@@H](C1)N2] |
| 506 | 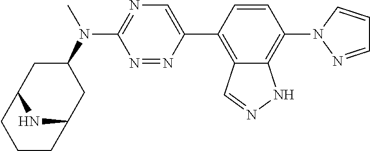 [SMILES: CN(c1ncc(-c2ccc(-n3cccn3)c3[nH]ncc23)nn1)[C@@H]1C[C@H]2CCC[C@@H](C1)N2] |
| 507 | 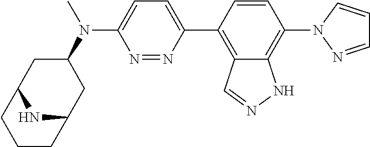 [SMILES: CN(c1ccc(-c2ccc(-n3cccn3)c3[nH]ncc23)nn1)[C@@H]1C[C@H]2CCC[C@@H](C1)N2] |
| 508 | 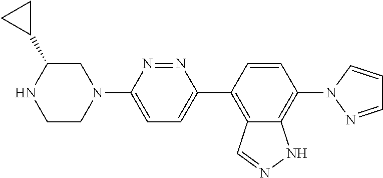 [SMILES: c1cnn(-c2ccc(-c3ccc(N4CCN[C@H](C5CC5)C4)nn3)c3cn[nH]c23)c1] |
| 509 | 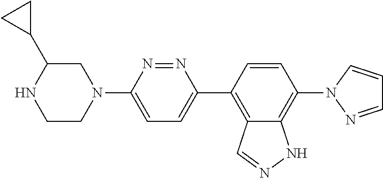 [SMILES: c1cnn(-c2ccc(-c3ccc(N4CCNC(C5CC5)C4)nn3)c3cn[nH]c23)c1] |
| 510 | 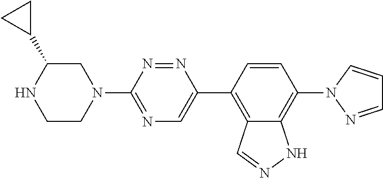 [SMILES: c1cnn(-c2ccc(-c3cnc(N4CCN[C@H](C5CC5)C4)nn3)c3cn[nH]c23)c1] |
| 511 | 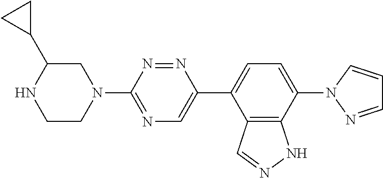 [SMILES: c1cnn(-c2ccc(-c3cnc(N4CCNC(C5CC5)C4)nn3)c3cn[nH]c23)c1] |
| 512 | 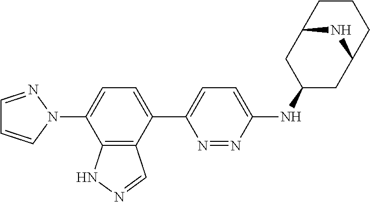 [SMILES: c1cnn(-c2ccc(-c3ccc(N[C@@H]4C[C@H]5CCC[C@@H](C4)N5)nn3)c3cn[nH]c23)c1] |
| 513 | 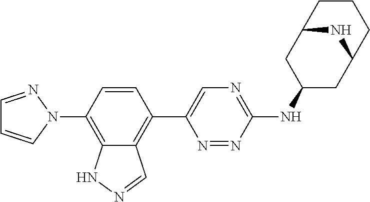 [SMILES: c1cnn(-c2ccc(-c3cnc(N[C@@H]4C[C@H]5CCC[C@@H](C4)N5)nn3)c3cn[nH]c23)c1] |
| 514 | 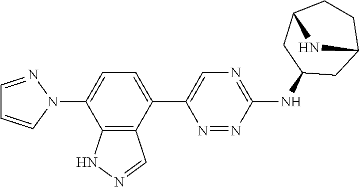 [SMILES: c1cnn(-c2ccc(-c3cnc(N[C@@H]4C[C@H]5CC[C@@H](C4)N5)nn3)c3cn[nH]c23)c1] |
| 515 | 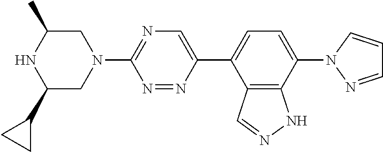 [SMILES: C[C@H]1CN(c2ncc(-c3ccc(-n4cccn4)c4[nH]ncc34)nn2)C[C@@H](C2CC2)N1] |
| 516 | 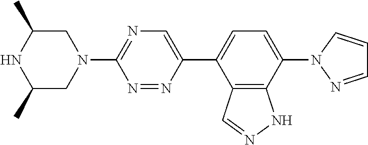 [SMILES: C[C@@H]1CN(c2ncc(-c3ccc(-n4cccn4)c4[nH]ncc34)nn2)C[C@H](C)N1] |
| 517 | 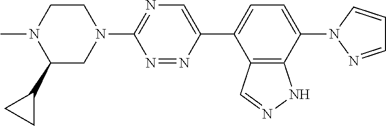 [SMILES: CN1CCN(c2ncc(-c3ccc(-n4cccn4)c4[nH]ncc34)nn2)C[C@H]1C1CC1] |
| 518 | 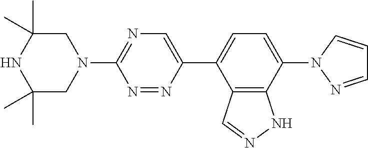 [SMILES: CC1(C)CN(c2ncc(-c3ccc(-n4cccn4)c4[nH]ncc34)nn2)CC(C)(C)N1] |
| 519 | 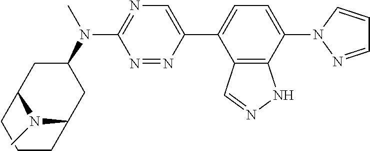 [SMILES: CN(c1ncc(-c2ccc(-n3cccn3)c3[nH]ncc23)nn1)[C@@H]1C[C@H]2CCC[C@@H](C1)N2C] |
| 520 | 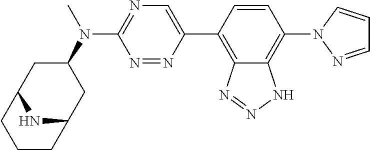 [SMILES: CN(c1ncc(-c2ccc(-n3cccn3)c3[nH]nnc23)nn1)[C@@H]1C[C@H]2CCC[C@@H](C1)N2] |
| 521 | 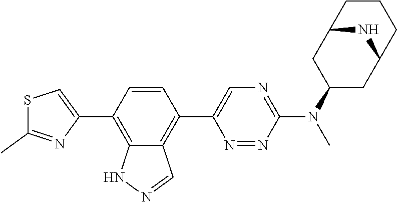 [SMILES: Cc1nc(-c2ccc(-c3cnc(N(C)[C@@H]4C[C@H]5CCC[C@@H](C4)N5)nn3)c3cn[nH]c23)cs1] |
| 522 | 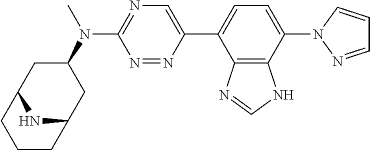 [SMILES: CN(c1ncc(-c2ccc(-n3cccn3)c3[nH]cnc23)nn1)[C@@H]1C[C@H]2CCC[C@@H](C1)N2] |
| 523 | 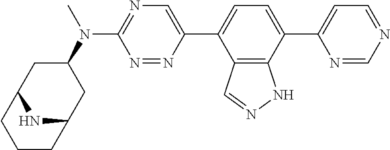 [SMILES: CN(c1ncc(-c2ccc(-c3ccncn3)c3[nH]ncc23)nn1)[C@@H]1C[C@H]2CCC[C@@H](C1)N2] |
| 524 | 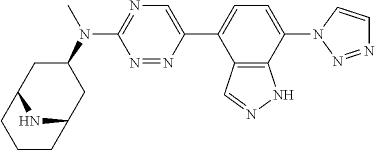 [SMILES: CN(c1ncc(-c2ccc(-n3ccnn3)c3[nH]ncc23)nn1)[C@@H]1C[C@H]2CCC[C@@H](C1)N2] |
| 525 | 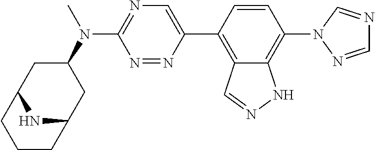 [SMILES: CN(c1ncc(-c2ccc(-n3cncn3)c3[nH]ncc23)nn1)[C@@H]1C[C@H]2CCC[C@@H](C1)N2] |
| 526 | 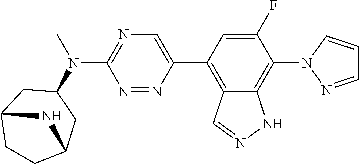 [SMILES: CN(c1ncc(-c2cc(F)c(-n3cccn3)c3[nH]ncc23)nn1)[C@@H]1C[C@H]2CC[C@@H](C1)N2] |
| 527 | 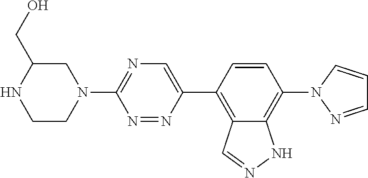 [SMILES: OCC1CN(c2ncc(-c3ccc(-n4cccn4)c4[nH]ncc34)nn2)CCN1] |
| 528 | 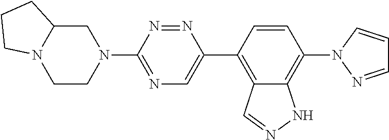 [SMILES: c1cnn(-c2ccc(-c3cnc(N4CCN5CCCC5C4)nn3)c3cn[nH]c23)c1] |
| 529 | 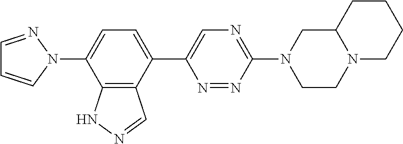 [SMILES: c1cnn(-c2ccc(-c3cnc(N4CCN5CCCCC5C4)nn3)c3cn[nH]c23)c1] |
| 530 | 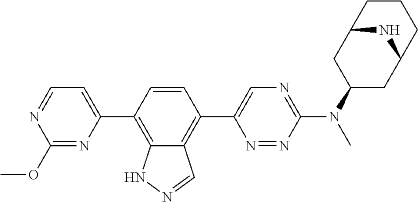 [SMILES: COc1nccc(-c2ccc(-c3cnc(N(C)[C@@H]4C[C@H]5CCC[C@@H](C4)N5)nn3)c3cn[nH]c23)n1] |
| 531 | 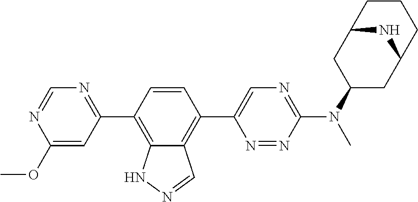 [SMILES: COc1cc(-c2ccc(-c3cnc(N(C)[C@@H]4C[C@H]5CCC[C@@H](C4)N5)nn3)c3cn[nH]c23)ncn1] |
| 532 | 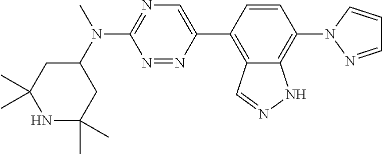 [SMILES: CN(c1ncc(-c2ccc(-n3cccn3)c3[nH]ncc23)nn1)C1CC(C)(C)NC(C)(C)C1] |
| 533 | 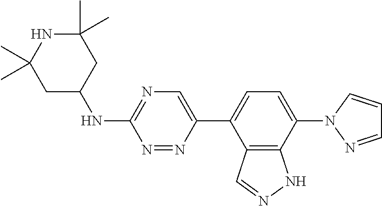 [SMILES: CC1(C)CC(Nc2ncc(-c3ccc(-n4cccn4)c4[nH]ncc34)nn2)CC(C)(C)N1] |
| 534 | 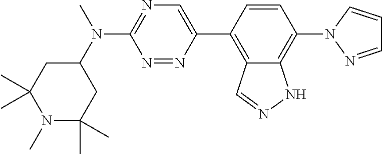 [SMILES: CN(c1ncc(-c2ccc(-n3cccn3)c3[nH]ncc23)nn1)C1CC(C)(C)N(C)C(C)(C)C1] |
| 535 | 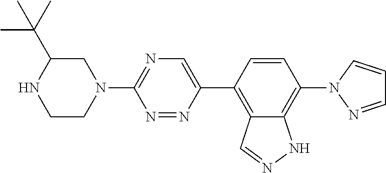 [SMILES: CC(C)(C)C1CN(c2ncc(-c3ccc(-n4cccn4)c4[nH]ncc34)nn2)CCN1] |
| 536 | 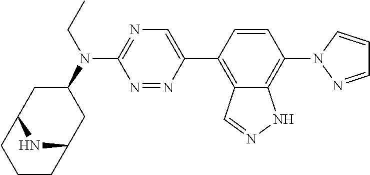 [SMILES: CCN(c1ncc(-c2ccc(-n3cccn3)c3[nH]ncc23)nn1)[C@@H]1C[C@H]2CCC[C@@H](C1)N2] |
| 537 | 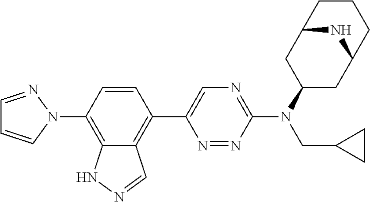 [SMILES: c1cnn(-c2ccc(-c3cnc(N(CC4CC4)[C@@H]4C[C@H]5CCC[C@@H](C4)N5)nn3)c3cn[nH]c23)c1] |
| 541 | 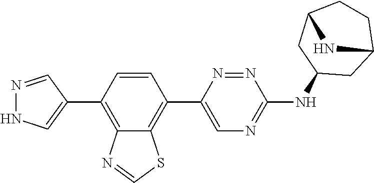 [SMILES: c1nc2c(-c3cn[nH]c3)ccc(-c3cnc(N[C@@H]4C[C@H]5CC[C@@H](C4)N5)nn3)c2s1] |
| 542 | 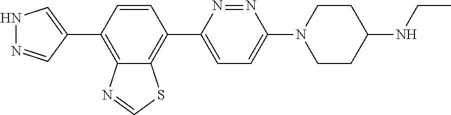 [SMILES: CCNC1CCN(c2ccc(-c3ccc(-c4cn[nH]c4)c4ncsc34)nn2)CC1] |
| 543 | 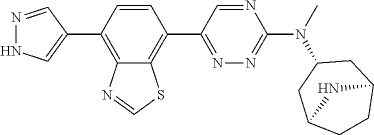 [SMILES: CN(c1ncc(-c2ccc(-c3cn[nH]c3)c3ncsc23)nn1)[C@@H]1C[C@H]2CC[C@@H](C1)N2] |
| 544 | 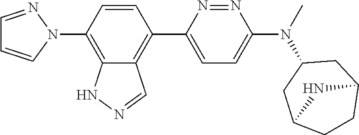 [SMILES: CN(c1ccc(-c2ccc(-n3cccn3)c3[nH]ncc23)nn1)[C@@H]1C[C@H]2CC[C@@H](C1)N2] |
| 545 | 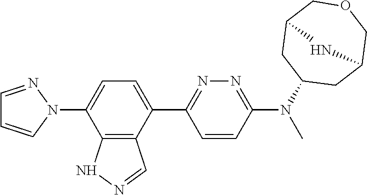 [SMILES: CN(c1ccc(-c2ccc(-n3cccn3)c3[nH]ncc23)nn1)[C@@H]1C[C@H]2COC[C@@H](C1)N2] |
| 546 | 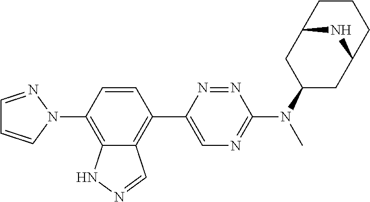 [SMILES: CN(c1ncc(-c2ccc(-n3cccn3)c3[nH]ncc23)nn1)[C@@H]1C[C@H]2CCC[C@@H](C1)N2] |
| 547 | 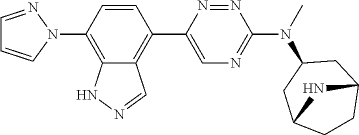 [SMILES: CN(c1ncc(-c2ccc(-n3cccn3)c3[nH]ncc23)nn1)[C@@H]1C[C@H]2CC[C@@H](C1)N2] |
| 548 | 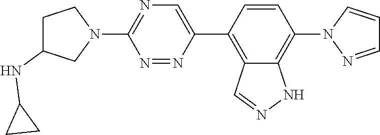 [SMILES: c1cnn(-c2ccc(-c3cnc(N4CCC(NC5CC5)C4)nn3)c3cn[nH]c23)c1] |
| 549 | 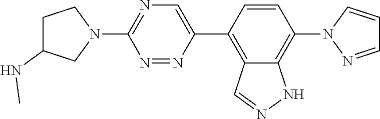 [SMILES: CNC1CCN(c2ncc(-c3ccc(-n4cccn4)c4[nH]ncc34)nn2)C1] |
| 550 | 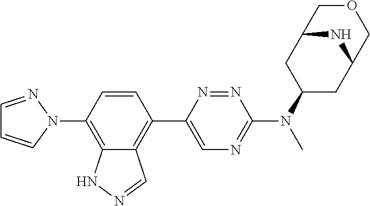 [SMILES: CN(c1ncc(-c2ccc(-n3cccn3)c3[nH]ncc23)nn1)[C@@H]1C[C@H]2COC[C@@H](C1)N2] |
| 551 | 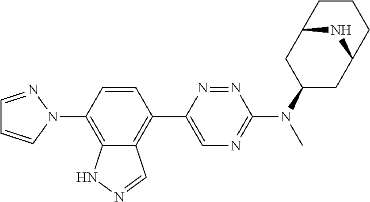 [SMILES: CN(c1ncc(-c2ccc(-n3cccn3)c3[nH]ncc23)nn1)[C@@H]1C[C@H]2CCC[C@@H](C1)N2] |
| 552 | 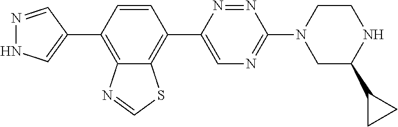 [SMILES: c1nc2c(-c3cn[nH]c3)ccc(-c3cnc(N4CCN[C@@H](C5CC5)C4)nn3)c2s1] |
| 553 | 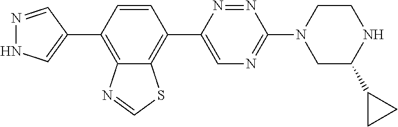 [SMILES: c1nc2c(-c3cn[nH]c3)ccc(-c3cnc(N4CCN[C@H](C5CC5)C4)nn3)c2s1] |
| 554 | 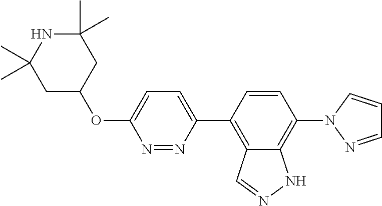 [SMILES: CC1(C)CC(Oc2ccc(-c3ccc(-n4cccn4)c4[nH]ncc34)nn2)CC(C)(C)N1] |
| 555 | 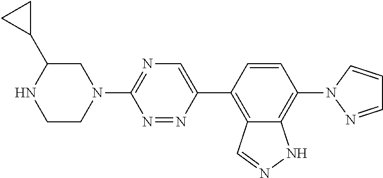 [SMILES: c1cnn(-c2ccc(-c3cnc(N4CCNC(C5CC5)C4)nn3)c3cn[nH]c23)c1] |
| 556 | 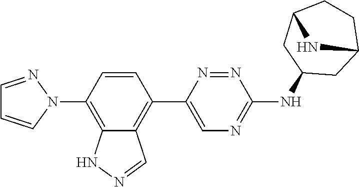 [SMILES: c1cnn(-c2ccc(-c3cnc(N[C@@H]4C[C@H]5CC[C@@H](C4)N5)nn3)c3cn[nH]c23)c1] |
| 557 | 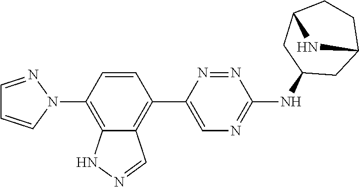 [SMILES: c1cnn(-c2ccc(-c3cnc(N[C@@H]4C[C@H]5CC[C@@H](C4)N5)nn3)c3cn[nH]c23)c1] |
| 558 | 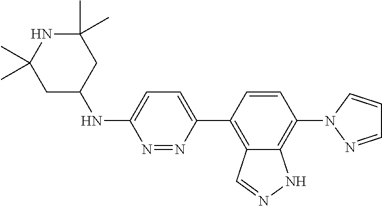 [SMILES: CC1(C)CC(Nc2ccc(-c3ccc(-n4cccn4)c4[nH]ncc34)nn2)CC(C)(C)N1] |
| 559 | 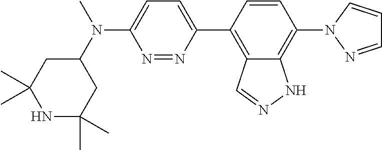 [SMILES: CN(c1ccc(-c2ccc(-n3cccn3)c3[nH]ncc23)nn1)C1CC(C)(C)NC(C)(C)C1] |
| 560 | 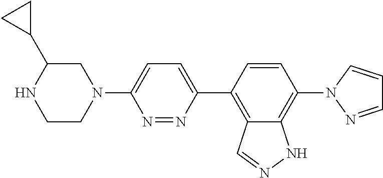 [SMILES: c1cnn(-c2ccc(-c3ccc(N4CCNC(C5CC5)C4)nn3)c3cn[nH]c23)c1] |
| 561 | 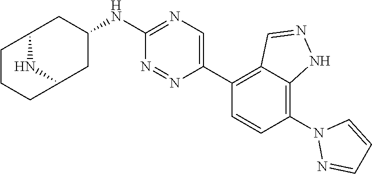 [SMILES: c1cnn(-c2ccc(-c3cnc(N[C@@H]4C[C@H]5CCC[C@@H](C4)N5)nn3)c3cn[nH]c23)c1] |
| 562 | 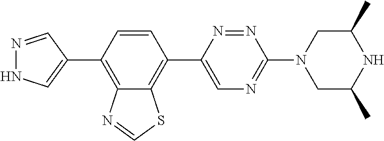 [SMILES: C[C@@H]1CN(c2ncc(-c3ccc(-c4cn[nH]c4)c4ncsc34)nn2)C[C@H](C)N1] |
| 563 | 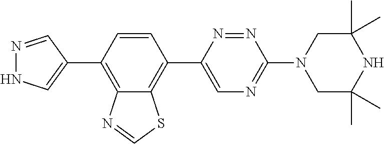 [SMILES: CC1(C)CN(c2ncc(-c3ccc(-c4cn[nH]c4)c4ncsc34)nn2)CC(C)(C)N1] |
| 564 | 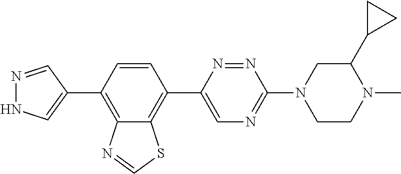 [SMILES: CN1CCN(c2ncc(-c3ccc(-c4cn[nH]c4)c4ncsc34)nn2)CC1C1CC1] |
| 565 | 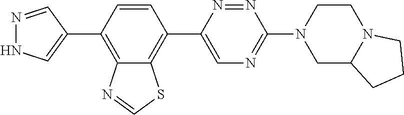 [SMILES: c1nc2c(-c3cn[nH]c3)ccc(-c3cnc(N4CCN5CCCC5C4)nn3)c2s1] |
| 566 | 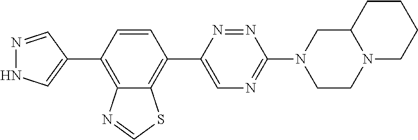 [SMILES: c1nc2c(-c3cn[nH]c3)ccc(-c3cnc(N4CCN5CCCCC5C4)nn3)c2s1] |
| 567 | 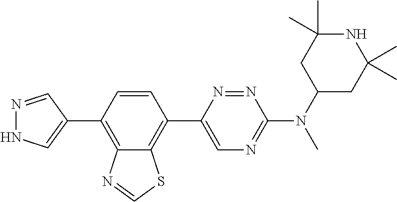 [SMILES: CN(c1ncc(-c2ccc(-c3cn[nH]c3)c3ncsc23)nn1)C1CC(C)(C)NC(C)(C)C1] |
| 568 | 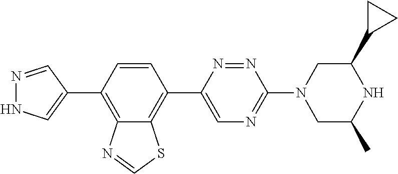 [SMILES: C[C@H]1CN(c2ncc(-c3ccc(-c4cn[nH]c4)c4ncsc34)nn2)C[C@@H](C2CC2)N1] |
| 569 | 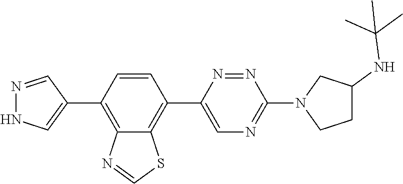 [SMILES: CC(C)(C)NC1CCN(c2ncc(-c3ccc(-c4cn[nH]c4)c4ncsc34)nn2)C1] |
| 570 | 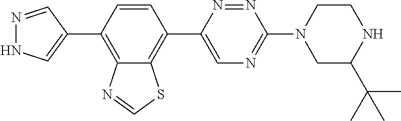 [SMILES: CC(C)(C)C1CN(c2ncc(-c3ccc(-c4cn[nH]c4)c4ncsc34)nn2)CCN1] |
| 571 | 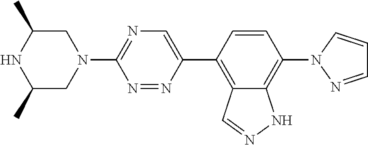 [SMILES: C[C@@H]1CN(c2ncc(-c3ccc(-n4cccn4)c4[nH]ncc34)nn2)C[C@H](C)N1] |
| 572 | 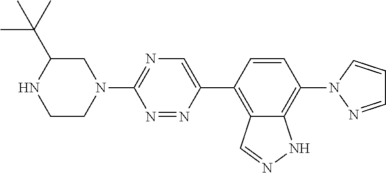 [SMILES: CC(C)(C)C1CN(c2ncc(-c3ccc(-n4cccn4)c4[nH]ncc34)nn2)CCN1] |
| 573 | 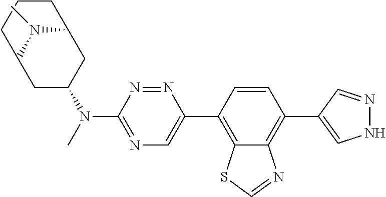 |
| 574 | 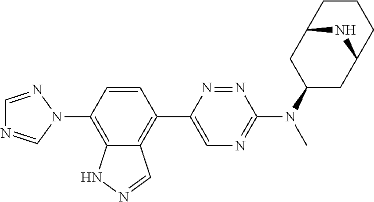 [SMILES: CN(c1ncc(-c2ccc(-n3cncn3)c3[nH]ncc23)nn1)[C@@H]1C[C@H]2CCC[C@@H](C1)N2] |
| 575 | 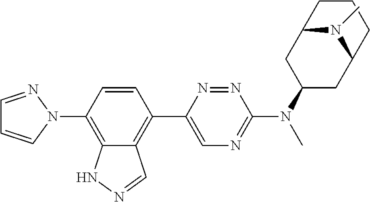 [SMILES: CN(c1ncc(-c2ccc(-n3cccn3)c3[nH]ncc23)nn1)[C@@H]1C[C@H]2CCC[C@@H](C1)N2C] |
| 576 | 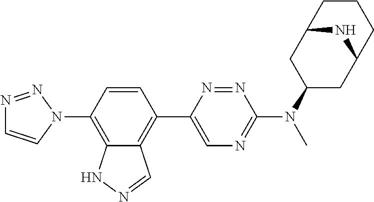 [SMILES: CN(c1ncc(-c2ccc(-n3ccnn3)c3[nH]ncc23)nn1)[C@@H]1C[C@H]2CCC[C@@H](C1)N2] |
| 577 | 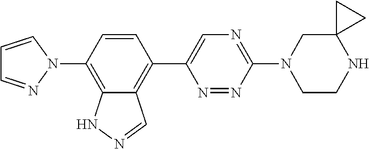 [SMILES: c1cnn(-c2ccc(-c3cnc(N4CCNC5(CC5)C4)nn3)c3cn[nH]c23)c1] |
| 578 | 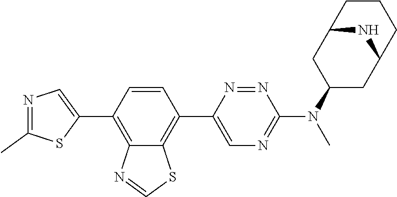 [SMILES: Cc1ncc(-c2ccc(-c3cnc(N(C)[C@@H]4C[C@H]5CCC[C@@H](C4)N5)nn3)c3scnc23)s1] |
| 579 | 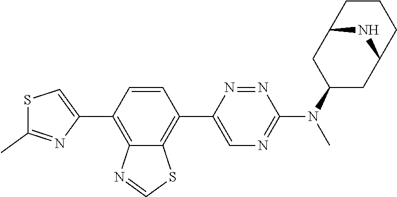 [SMILES: Cc1nc(-c2ccc(-c3cnc(N(C)[C@@H]4C[C@H]5CCC[C@@H](C4)N5)nn3)c3scnc23)cs1] |
| 580 | 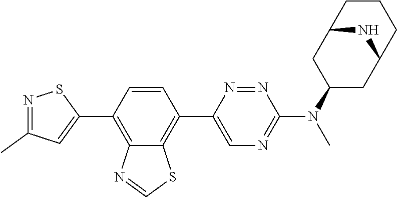 [SMILES: Cc1cc(-c2ccc(-c3cnc(N(C)[C@@H]4C[C@H]5CCC[C@@H](C4)N5)nn3)c3scnc23)sn1] |
| 581 | 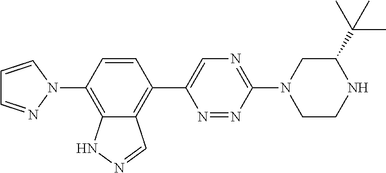 [SMILES: CC(C)(C)[C@H]1CN(c2ncc(-c3ccc(-n4cccn4)c4[nH]ncc34)nn2)CCN1] |
| 582 | 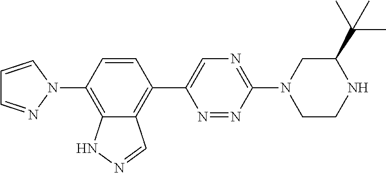 [SMILES: CC(C)(C)[C@@H]1CN(c2ncc(-c3ccc(-n4cccn4)c4[nH]ncc34)nn2)CCN1] |
| 583 | 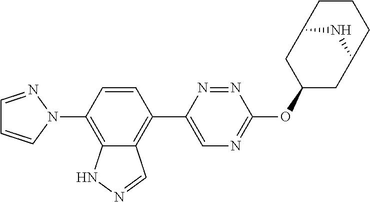 [SMILES: c1cnn(-c2ccc(-c3cnc(O[C@H]4C[C@H]5CCC[C@@H](C4)N5)nn3)c3cn[nH]c23)c1] |
| 584 | 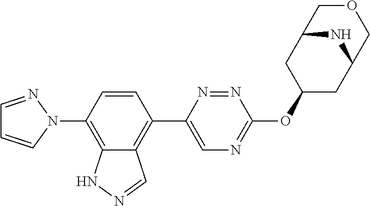 [SMILES: c1cnn(-c2ccc(-c3cnc(O[C@@H]4C[C@H]5COC[C@@H](C4)N5)nn3)c3cn[nH]c23)c1] |
| 585 | 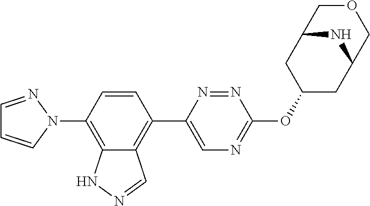 [SMILES: c1cnn(-c2ccc(-c3cnc(O[C@H]4C[C@H]5COC[C@@H](C4)N5)nn3)c3cn[nH]c23)c1] |
| 586 | 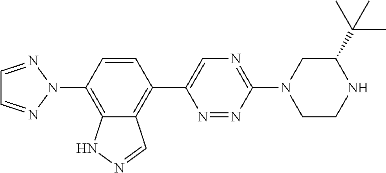 [SMILES: CC(C)(C)[C@H]1CN(c2ncc(-c3ccc(-n4nccn4)c4[nH]ncc34)nn2)CCN1] |
| 587 | 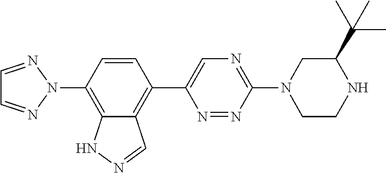 [SMILES: CC(C)(C)[C@@H]1CN(c2ncc(-c3ccc(-n4nccn4)c4[nH]ncc34)nn2)CCN1] |
| 588 | 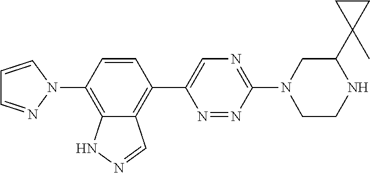 [SMILES: CC1(C2CN(c3ncc(-c4ccc(-n5cccn5)c5[nH]ncc45)nn3)CCN2)CC1] |
| 589 | 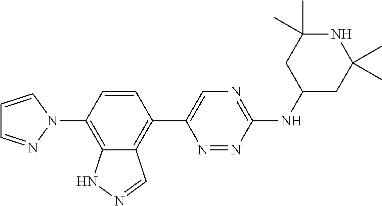 [SMILES: CC1(C)CC(Nc2ncc(-c3ccc(-n4cccn4)c4[nH]ncc34)nn2)CC(C)(C)N1] |
| 590 | 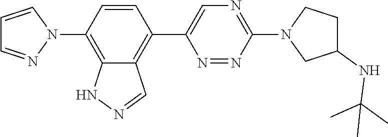 [SMILES: CC(C)(C)NC1CCN(c2ncc(-c3ccc(-n4cccn4)c4[nH]ncc34)nn2)C1] |
| 591 | 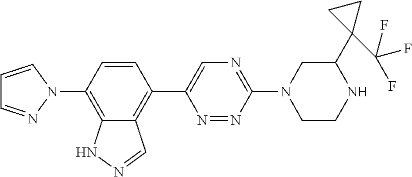 [SMILES: FC(F)(F)C1(C2CN(c3ncc(-c4ccc(-n5cccn5)c5[nH]ncc45)nn3)CCN2)CC1] |
| 592 | 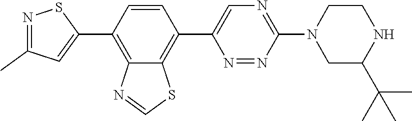 [SMILES: Cc1cc(-c2ccc(-c3cnc(N4CCNC(C(C)(C)C)C4)nn3)c3scnc23)sn1] |
| 593 | 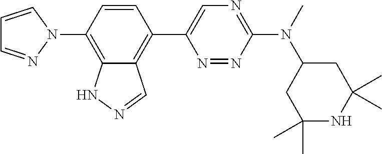 [SMILES: CN(c1ncc(-c2ccc(-n3cccn3)c3[nH]ncc23)nn1)C1CC(C)(C)NC(C)(C)C1] |
| 594 | 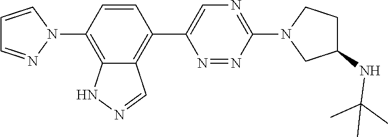 [SMILES: CC(C)(C)N[C@@H]1CCN(c2ncc(-c3ccc(-n4cccn4)c4[nH]ncc34)nn2)C1] |
| 595 | 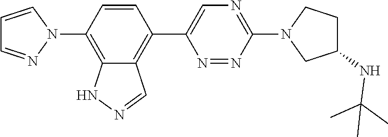 [SMILES: CC(C)(C)N[C@H]1CCN(c2ncc(-c3ccc(-n4cccn4)c4[nH]ncc34)nn2)C1] |
| 596 | 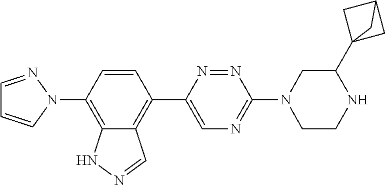 [SMILES: c1cnn(-c2ccc(-c3cnc(N4CCNC(C56CC(C5)C6)C4)nn3)c3cn[nH]c23)c1] |
| 597 | 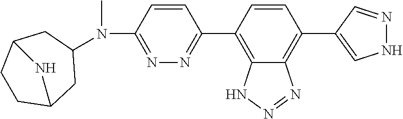 [SMILES: CN(c1ccc(-c2ccc(-c3cn[nH]c3)c3nn[nH]c23)nn1)C1CC2CCC(C1)N2] |
| 598 | 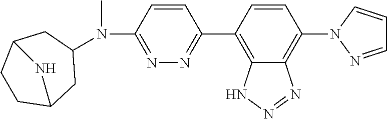 [SMILES: CN(c1ccc(-c2ccc(-n3cccn3)c3nn[nH]c23)nn1)C1CC2CCC(C1)N2] |
| 599 | 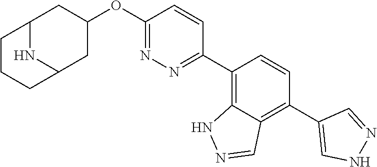 [SMILES: c1n[nH]cc1-c1ccc(-c2ccc(OC3CC4CCCC(C3)N4)nn2)c2[nH]ncc12] |
| 600 | 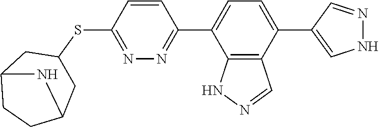 [SMILES: c1n[nH]cc1-c1ccc(-c2ccc(SC3CC4CCC(C3)N4)nn2)c2[nH]ncc12] |
| 601 | 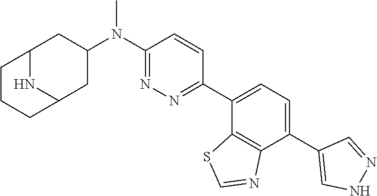 [SMILES: CN(c1ccc(-c2ccc(-c3cn[nH]c3)c3ncsc23)nn1)C1CC2CCCC(C1)N2] |
| 602 | 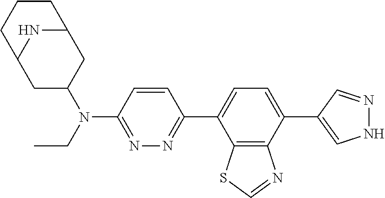 [SMILES: CCN(c1ccc(-c2ccc(-c3cn[nH]c3)c3ncsc23)nn1)C1CC2CCCC(C1)N2] |
| 603 | 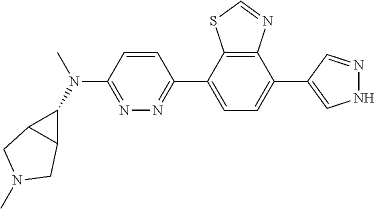 |
| 604 | 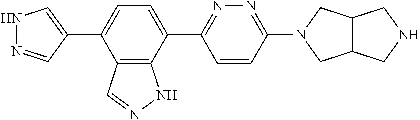 [SMILES: c1n[nH]cc1-c1ccc(-c2ccc(N3CC4CNCC4C3)nn2)c2[nH]ncc12] |
| 605 | 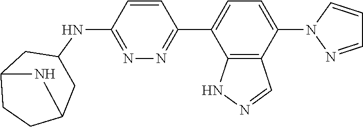 [SMILES: c1cnn(-c2ccc(-c3ccc(NC4CC5CCC(C4)N5)nn3)c3[nH]ncc23)c1] |
| 606 | 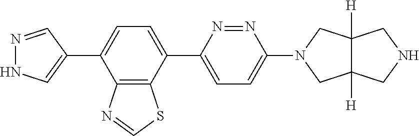 [SMILES: c1nc2c(-c3cn[nH]c3)ccc(-c3ccc(N4CC5CNCC5C4)nn3)c2s1] |
| 607 | 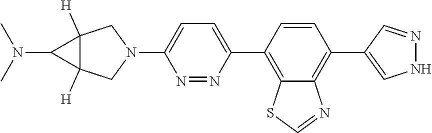 [SMILES: CN(C)C1C2CN(c3ccc(-c4ccc(-c5cn[nH]c5)c5ncsc45)nn3)CC21] |
| 608 | 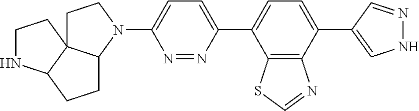 [SMILES: c1nc2c(-c3cn[nH]c3)ccc(-c3ccc(N4CCC56CCNC5CCC46)nn3)c2s1] |
| 609 | 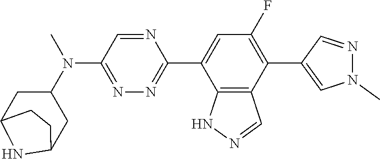 [SMILES: CN(c1cnc(-c2cc(F)c(-c3cnn(C)c3)c3cn[nH]c23)nn1)C1CC2CCC(C1)N2] |
| 610 | 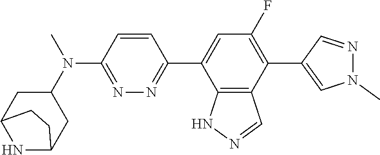 [SMILES: CN(c1ccc(-c2cc(F)c(-c3cnn(C)c3)c3cn[nH]c23)nn1)C1CC2CCC(C1)N2] |
| 611 | 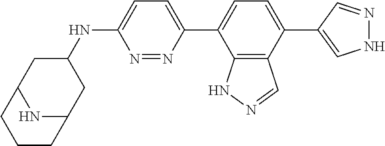 [SMILES: c1n[nH]cc1-c1ccc(-c2ccc(NC3CC4CCCC(C3)N4)nn2)c2[nH]ncc12] |
| 612 | 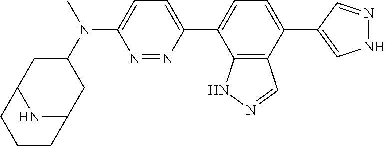 [SMILES: CN(c1ccc(-c2ccc(-c3cn[nH]c3)c3cn[nH]c23)nn1)C1CC2CCCC(C1)N2] |
| 613 | 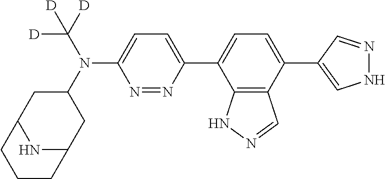 [SMILES: [2H]C([2H])([2H])N(c1ccc(-c2ccc(-c3cn[nH]c3)c3cn[nH]c23)nn1)C1CC2CCCC(C1)N2] |
| 614 | 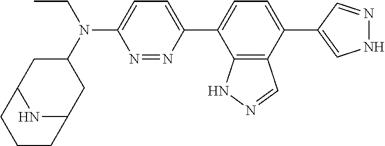 [SMILES: CCN(c1ccc(-c2ccc(-c3cn[nH]c3)c3cn[nH]c23)nn1)C1CC2CCCC(C1)N2] |
| 615 | 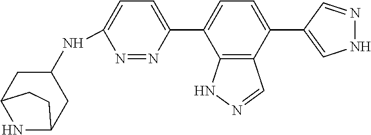 [SMILES: c1n[nH]cc1-c1ccc(-c2ccc(NC3CC4CCC(C3)N4)nn2)c2[nH]ncc12] |
| 616 | 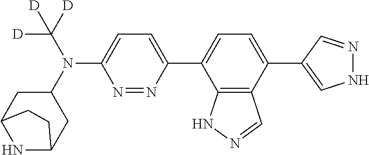 [SMILES: [2H]C([2H])([2H])N(c1ccc(-c2ccc(-c3cn[nH]c3)c3cn[nH]c23)nn1)C1CC2CCC(C1)N2] |
| 617 | 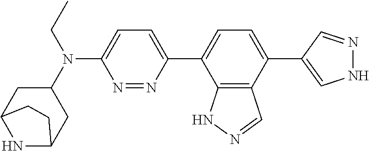 [SMILES: CCN(c1ccc(-c2ccc(-c3cn[nH]c3)c3cn[nH]c23)nn1)C1CC2CCC(C1)N2] |
| 618 | 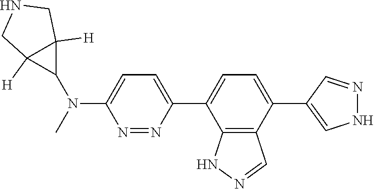 [SMILES: CN(c1ccc(-c2ccc(-c3cn[nH]c3)c3cn[nH]c23)nn1)C1C2CNCC21] |
| 619 | 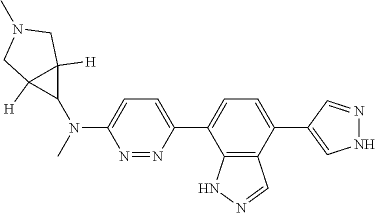 [SMILES: CN1CC2C(C1)C2N(C)c1ccc(-c2ccc(-c3cn[nH]c3)c3cn[nH]c23)nn1] |
| 620 | 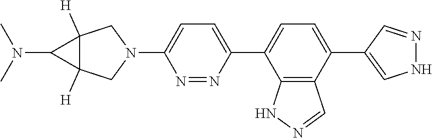 [SMILES: CN(C)C1C2CN(c3ccc(-c4ccc(-c5cn[nH]c5)c5cn[nH]c45)nn3)CC21] |
| 621 | 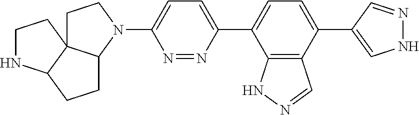 [SMILES: c1n[nH]cc1-c1ccc(-c2ccc(N3CCC45CCNC4CCC35)nn2)c2[nH]ncc12] |
| 622 | 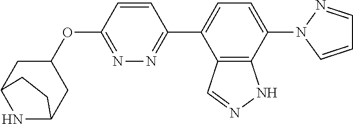 [SMILES: c1cnn(-c2ccc(-c3ccc(OC4CC5CCC(C4)N5)nn3)c3cn[nH]c23)c1] |
| 623 | 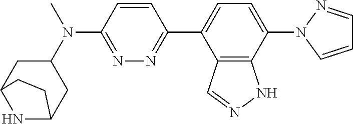 [SMILES: CN(c1ccc(-c2ccc(-n3cccn3)c3[nH]ncc23)nn1)C1CC2CCC(C1)N2] |
| 624 | 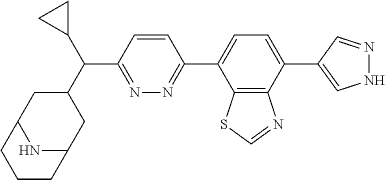 [SMILES: c1nc2c(-c3cn[nH]c3)ccc(-c3ccc(C(C4CC4)C4CC5CCCC(C4)N5)nn3)c2s1] |
| 625 | 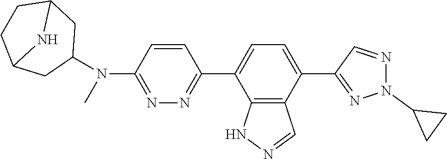 [SMILES: CN(c1ccc(-c2ccc(-c3cnn(C4CC4)n3)c3cn[nH]c23)nn1)C1CC2CCC(C1)N2] |
| 626 | 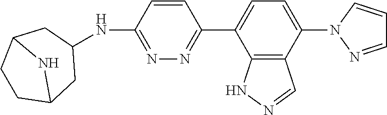 [SMILES: c1cnn(-c2ccc(-c3ccc(NC4CC5CCC(C4)N5)nn3)c3[nH]ncc23)c1] |
| 627 | 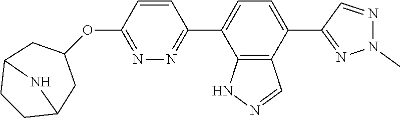 [SMILES: Cn1ncc(-c2ccc(-c3ccc(OC4CC5CCC(C4)N5)nn3)c3[nH]ncc23)n1] |
| 628 | 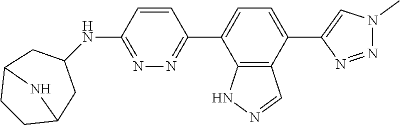 [SMILES: Cn1cc(-c2ccc(-c3ccc(NC4CC5CCC(C4)N5)nn3)c3[nH]ncc23)nn1] |
| 629 | 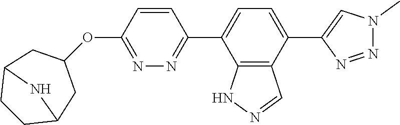 [SMILES: Cn1cc(-c2ccc(-c3ccc(OC4CC5CCC(C4)N5)nn3)c3[nH]ncc23)nn1] |
| 630 | 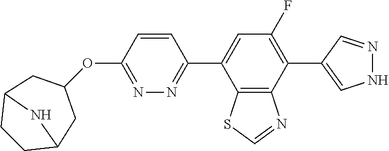 [SMILES: Fc1cc(-c2ccc(OC3CC4CCC(C3)N4)nn2)c2scnc2c1-c1cn[nH]c1] |
| 631 | 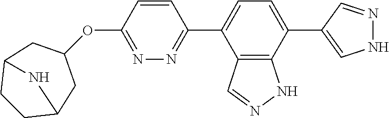 [SMILES: c1n[nH]cc1-c1ccc(-c2ccc(OC3CC4CCC(C3)N4)nn2)c2cn[nH]c12] |
| 632 | 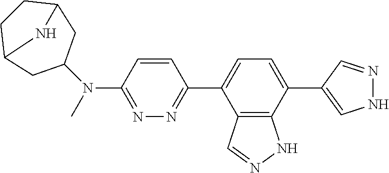 [SMILES: CN(c1ccc(-c2ccc(-c3cn[nH]c3)c3[nH]ncc23)nn1)C1CC2CCC(C1)N2] |
| 633 | 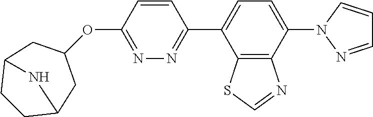 [SMILES: c1cnn(-c2ccc(-c3ccc(OC4CC5CCC(C4)N5)nn3)c3scnc23)c1] |
| 634 | 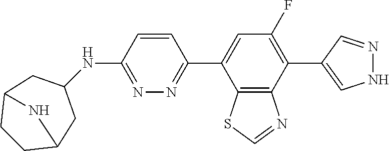 [SMILES: Fc1cc(-c2ccc(NC3CC4CCC(C3)N4)nn2)c2scnc2c1-c1cn[nH]c1] |
| 635 | 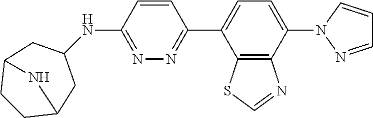 [SMILES: c1cnn(-c2ccc(-c3ccc(NC4CC5CCC(C4)N5)nn3)c3scnc23)c1] |
| 636 | 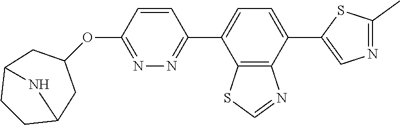 [SMILES: Cc1ncc(-c2ccc(-c3ccc(OC4CC5CCC(C4)N5)nn3)c3scnc23)s1] |
| 637 | 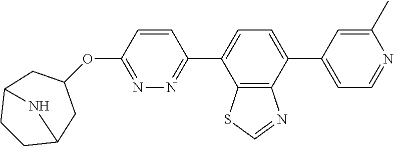 [SMILES: Cc1cc(-c2ccc(-c3ccc(OC4CC5CCC(C4)N5)nn3)c3scnc23)ccn1] |
| 638 | 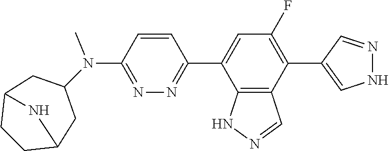 [SMILES: CN(c1ccc(-c2cc(F)c(-c3cn[nH]c3)c3cn[nH]c23)nn1)C1CC2CCC(C1)N2] |
| 639 | 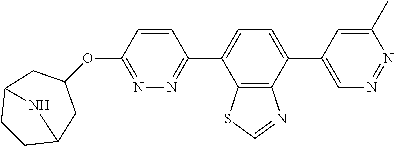 [SMILES: Cc1cc(-c2ccc(-c3ccc(OC4CC5CCC(C4)N5)nn3)c3scnc23)cnn1] |
| 640 | 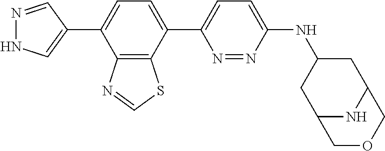 [SMILES: c1nc2c(-c3cn[nH]c3)ccc(-c3ccc(NC4CC5COCC(C4)N5)nn3)c2s1] |
| 641 | 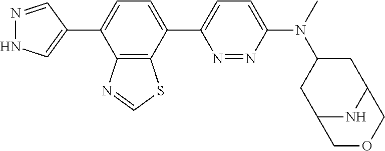 [SMILES: CN(c1ccc(-c2ccc(-c3cn[nH]c3)c3ncsc23)nn1)C1CC2COCC(C1)N2] |
| 642 | 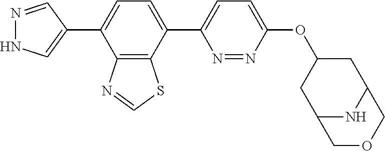 [SMILES: c1nc2c(-c3cn[nH]c3)ccc(-c3ccc(OC4CC5COCC(C4)N5)nn3)c2s1] |
| 643 | 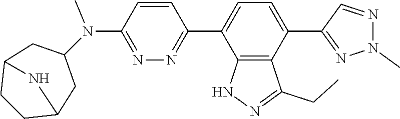 [SMILES: CCc1n[nH]c2c(-c3ccc(N(C)C4CC5CCC(C4)N5)nn3)ccc(-c3cnn(C)n3)c12] |
| 644 | 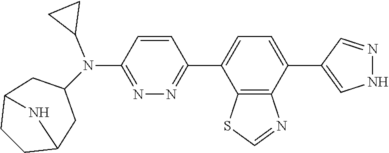 [SMILES: c1nc2c(-c3cn[nH]c3)ccc(-c3ccc(N(C4CC4)C4CC5CCC(C4)N5)nn3)c2s1] |
| 645 | 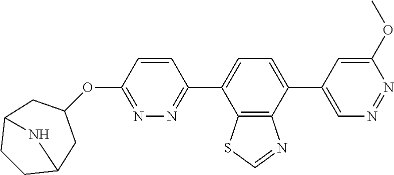 [SMILES: COc1cc(-c2ccc(-c3ccc(OC4CC5CCC(C4)N5)nn3)c3scnc23)cnn1] |
| 646 | 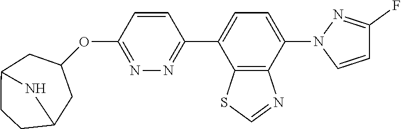 [SMILES: Fc1ccn(-c2ccc(-c3ccc(OC4CC5CCC(C4)N5)nn3)c3scnc23)n1] |
| 647 | 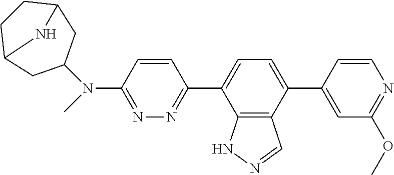 [SMILES: COc1cc(-c2ccc(-c3ccc(N(C)C4CC5CCC(C4)N5)nn3)c3[nH]ncc23)ccn1] |
| 648 | 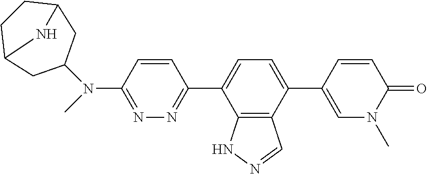 [SMILES: CN(c1ccc(-c2ccc(-c3ccc(=O)n(C)c3)c3cn[nH]c23)nn1)C1CC2CCC(C1)N2] |
| 649 | 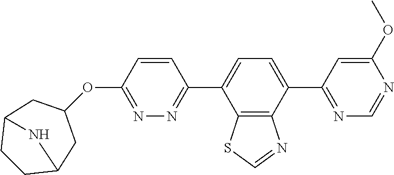 [SMILES: COc1cc(-c2ccc(-c3ccc(OC4CC5CCC(C4)N5)nn3)c3scnc23)ncn1] |
| 650 | 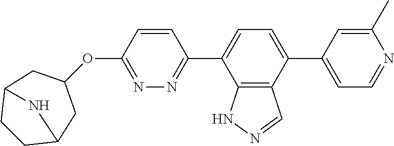 [SMILES: Cc1cc(-c2ccc(-c3ccc(OC4CC5CCC(C4)N5)nn3)c3[nH]ncc23)ccn1] |
| 651 | 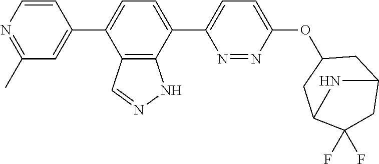 [SMILES: Cc1cc(-c2ccc(-c3ccc(OC4CC5CC(F)(F)C(C4)N5)nn3)c3[nH]ncc23)ccn1] |
| 652 | 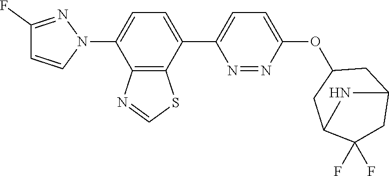 [SMILES: Fc1ccn(-c2ccc(-c3ccc(OC4CC5CC(F)(F)C(C4)N5)nn3)c3scnc23)n1] |
| 653 | 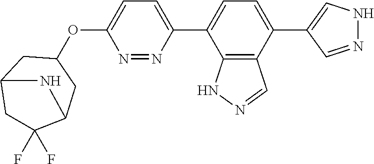 [SMILES: FC1(F)CC2CC(Oc3ccc(-c4ccc(-c5cn[nH]c5)c5cn[nH]c45)nn3)CC1N2] |
| 654 | 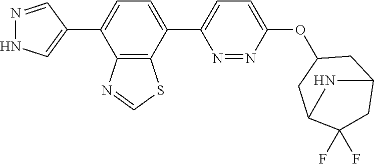 [SMILES: FC1(F)CC2CC(Oc3ccc(-c4ccc(-c5cn[nH]c5)c5ncsc45)nn3)CC1N2] |
| 655 | 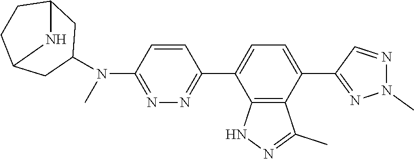 [SMILES: Cc1n[nH]c2c(-c3ccc(N(C)C4CC5CCC(C4)N5)nn3)ccc(-c3cnn(C)n3)c12] |
| 656 | 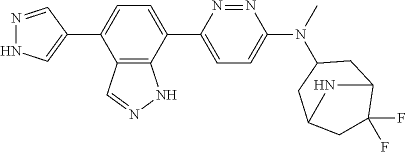 [SMILES: CN(c1ccc(-c2ccc(-c3cn[nH]c3)c3cn[nH]c23)nn1)C1CC2CC(F)(F)C(C1)N2] |
| 657 | 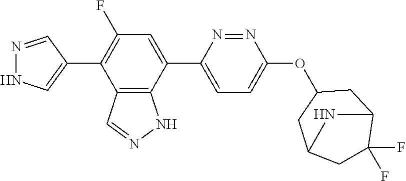 [SMILES: Fc1cc(-c2ccc(OC3CC4CC(F)(F)C(C3)N4)nn2)c2[nH]ncc2c1-c1cn[nH]c1] |
| 658 | 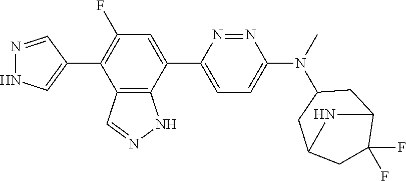 [SMILES: CN(c1ccc(-c2cc(F)c(-c3cn[nH]c3)c3cn[nH]c23)nn1)C1CC2CC(F)(F)C(C1)N2] |
| 659 | 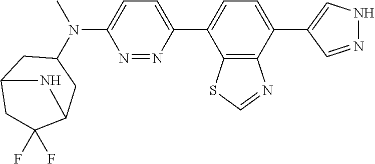 [SMILES: CN(c1ccc(-c2ccc(-c3cn[nH]c3)c3ncsc23)nn1)C1CC2CC(F)(F)C(C1)N2] |
| 660 | 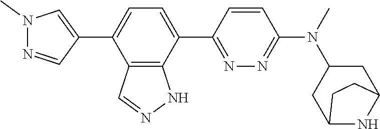 [SMILES: CN(c1ccc(-c2ccc(-c3cnn(C)c3)c3cn[nH]c23)nn1)C1CC2CCC(C1)N2] |
| 661 | 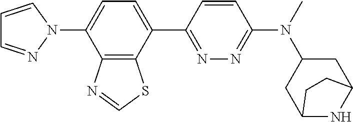 [SMILES: CN(c1ccc(-c2ccc(-n3cccn3)c3ncsc23)nn1)C1CC2CCC(C1)N2] |

In some embodiments, for Formula (I), A is monocyclic heterocyclyl (e.g., 2-methylpiperidinyl); B is monocyclic heteroaryl (e.g., pyrazolyl); L is —N(R³)— (e.g., —N(CH³)—); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 100, Compound 101, Compound 102, Compound 103, Compound 104, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —N(CH³)—); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 104, Compound 105, or a pharmaceutically acceptable salt, solvate, hydrate, tautomer, or stereoisomer thereof.

In some embodiments, for Formula (I), A is monocyclic heterocyclyl (e.g., 2-methylpiperidinyl); B is monocyclic heteroaryl (e.g., pyrazolyl); L is —N(R³)— (e.g., N(CH³)); M and P are each C(R²) (e.g., CH); R⁷ is absent; D is N(R^{5c}) (e.g., NH); E and F are independently C(R^{5a})(e.g., CH). In some embodiments, the compound of Formula (I) is Compound 106, Compound 107, Compound 108, Compound 109, Compound 110, Compound 111 or a pharmaceutically acceptable salt, solvate, hydrate, tautomer, or stereoisomer thereof.

In some embodiments, for Formula (I), A is monocyclic heterocyclyl (e.g., 8-azabicyclo[3.2.1]octanyl); B is monocyclic heteroaryl (e.g., pyrazolyl); L is —N(R³)— (e.g., —N(CH³)—); M and P are each C(R²) (e.g., CH); R⁷ is absent; D is N(R^{5c}) (e.g., NH); E and F are independently C(R^{5a})(e.g., CH). In some embodiments, the compound of Formula (I) is Compound 112, Compound 113 or a pharmaceutically acceptable salt, solvate, hydrate, tautomer, or stereoisomer thereof.

In some embodiments, for Formula (I), A is bicyclic heterocyclyl (e.g., 2-methylpiperidinyl); B is monocyclic heteroaryl (e.g., pyrazolyl); L is —N(R³)— (e.g., —N(CH³)—); M and P are each C(R²) (e.g., CH); R⁷ is absent; D is N(R^{5c}) (e.g., NH); and E and F are each independently N. In some embodiments, the compound of Formula (I) is Compound 114, Compound 115, Compound 116, Compound 117 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., N(CH³)); M and P are each C(R²) (e.g., CH); R⁷ is absent; D is N(R^{5c}) (e.g., NH); and E and F are each independently N. In some embodiments, the compound of Formula (I) is Compound 118, Compound 119, or a pharmaceutically acceptable salt, solvate, hydrate, tautomer, or stereoisomer thereof.

In some embodiments, for Formula (I), A is bicyclic heterocyclyl (e.g., 2-methylpiperidinyl); B is monocyclic heteroaryl (e.g., pyrazolyl); L is —N(R³)— (e.g., —N(CH³)—); M and P are each C(R²) (e.g., CH); R⁷ is absent; D is N(R^{5c}) (e.g., NH); E is C(R^{5a})(e.g., —CO); and F is C(R^{5a})(e.g., CH₂). In some embodiments, the compound of Formula (I) is Compound 120, Compound 121, Compound 122, or a pharmaceutically acceptable salt, solvate, hydrate, tautomer, or stereoisomer thereof.

In some embodiments, for Formula (I), A is bicyclic heterocyclyl (e.g., (1R,5S)-8-azabicyclo[3.2.1]octanyl); B is monocyclic heteroaryl (e.g., pyrazolyl); L is —N(R³)— (e.g., N(CH³)); M and P are each C(R²) (e.g., CH); R⁷ is absent; D is N(R^{5c}) (e.g., NH); E is C(R^{5a})(e.g., —CO); and F is C(R^{5a})(e.g., CH₂). In some embodiments, the compound of Formula (I) is Compound 123, Compound 124, or a pharmaceutically acceptable salt, solvate, hydrate, tautomer, or stereoisomer thereof.

In some embodiments, for Formula (I), A is bicyclic heterocyclyl (e.g., 2, 2, 6, 6-tetramethylpiperidinyl); B is monocyclic heteroaryl (e.g., pyrazolyl); L is —N(R³)— (e.g., N(CH³)); M and P are each C(R²) (e.g., CH); R⁷ is absent; D is S; and E and F are each independently C(R^{5a})(e.g., CH). In some embodiments, the compound of Formula (I) is Compound 131, or a pharmaceutically acceptable salt, solvate, hydrate, tautomer, or stereoisomer thereof.

In some embodiments, for Formula (I), A is bicyclic heterocyclyl (e.g., 2, 2, 6, 6-tetramethylpiperidinyl); B is monocyclic heteroaryl (e.g., pyrazolyl); L is —N(R³)— (e.g., —N(CH³)—); M and P are each C(R²) (e.g., CH); R⁷ is absent; D is S; E is C(R^{5a})(e.g., CH); and F is N. In some embodiments, the compound of Formula (I) is Compound 132, or a pharmaceutically acceptable salt, solvate, hydrate, tautomer, or stereoisomer thereof.

In some embodiments, for Formula (I), A is monocyclic heterocyclyl (e.g., 2, 2, 6, 6-tetramethylpiperidinyl); B is monocyclic heteroaryl (e.g., pyrazolyl); L is —N(R³)— (e.g., N(CH³)); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 137 or a pharmaceutically acceptable salt, solvate, hydrate, tautomer, or stereoisomer thereof.

In some embodiments, for Formula (I), A is monocyclic heterocyclyl (e.g., 2, 2, 6, 6-tetramethylpiperidinyl); B is monocyclic heteroaryl (e.g., pyrazolyl); L is —O—; M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 138 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1, 2, 4-triazolyl); L is —N(R³)— (e.g., N(CH³)); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 139, Compound 140, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1, 2, 3-triazolyl); L is —N(R³)— (e.g., —N(CH³)—); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 141, Compound 164, or a pharmaceutically acceptable salt, solvate, hydrate, tautomer, or stereoisomer thereof.

In some embodiments, for Formula (I), A is monocyclic heterocyclyl (e.g., 8-azabicyclo[3.2.1]octanyl); B is monocyclic heteroaryl (e.g., pyrazolyl); L is —O—; M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 142 or a pharmaceutically acceptable salt, solvate, hydrate, tautomer, or stereoisomer thereof.

In some embodiments, for Formula (I), A is monocyclic heterocyclyl (e.g., 8-azabicyclo[3.2.1]octanyl); B is monocyclic heteroaryl (e.g., methylpyrazolyl); L is —N(R³)— (e.g., —N(CH³)—); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 143 or a pharmaceutically acceptable salt, solvate, hydrate, tautomer, or stereoisomer thereof. In some embodiments, for Formula (I), A is monocyclic heterocyclyl (e.g., 8-azabicyclo[3.2.1]octanyl); B is monocyclic heteroaryl (e.g., 2-methyl-1,2,3-triazolyl); L is —N(R³)— (e.g., N(CH³)); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 144, Compound 165, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1,2,4-triazolyl); L is —N(R³)— (e.g., N(CH³)); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 145, or a pharmaceutically acceptable salt, solvate, hydrate, tautomer, or stereoisomer thereof.

In some embodiments, for Formula (I), A is bicyclic heterocyclyl (e.g., 1,5-dimethyl-8-azabicyclo[3.2.1]octanyl); B is monocyclic heteroaryl (e.g., pyrazolyl); L is —N(R³)— (e.g., —N(CH³)—); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 146, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g, N(CH³)); M and P are each C(R²) (e.g., CH); R⁷ is halo (e.g. F); D is N(R^{5c}) (e.g., NH); E is N; F is C(R^{5a})(e.g., CH); and n is 1. In some embodiments, the compound of Formula (I) is Compound 147, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., N(CH³)); M is C(R²) (e.g., CH); P is N; R⁷ is absent; D is N(R^{5c}) (e.g., NH); E is N; and F is C(R^{5a})(e.g., CH). In some embodiments, the compound of Formula (I) is Compound 148, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g, N(CH³)); M is C(R²) (e.g., CH); P is N; R⁷ is halo (e.g. F); D is N(R^{5c}) (e.g., NH); E is N; F is C(R^{5a})(e.g., CH); and n is 1. In some embodiments, the compound of Formula (I) is Compound 149, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R³)— (e.g., N(CH³)); M and P are each independently C(R²) (e.g., CH); R⁷ is absent; D is N(R^{5c}) (e.g., NH); E is N; F is C(R^{5a})(e.g., CH). In some embodiments, the compound of Formula (I) is Compound 150, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —O—; M and P are each independently C(R²) (e.g., CH); R⁷ is absent; D is N(R^{5c}) (e.g., NH); E is N; F is C(R^{5a})(e.g., CH). In some embodiments, the compound of Formula (I) is Compound 151, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —O—; M and P are each independently C(R²) (e.g., CH); R⁷ is absent; D is N(R^{5c}) (e.g., NH); E is N; F is C(R^{5a})(e.g., CH). In some embodiments, the compound of Formula (I) is Compound 152, Compound 168 or a pharmaceutically acceptable salt, solvate, hydrate, tautomer, or stereoisomer thereof.

In some embodiments, for Formula (I), A is bicyclic heterocyclyl (e.g., 1,5-dimethyl-8-azabicyclo[3.2.1]octanyl); B is monocyclic heteroaryl (e.g., pyrazolyl); L is —N(R³)— (e.g., N(CH³)); M and P are each independently C(R²) (e.g., CH); R⁷ is absent; D is N(R^{5c}) (e.g., NH); E is N; F is C(R^{5a})(e.g., CH). In some embodiments, the compound of Formula (I) is Compound 153, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., N(CH³)); M and P are each independently C(R²) (e.g., CH); R⁷ is halo (e.g. F); D is N(R^{5c}) (e.g., NH); E is N; F is C(R^{5a})(e.g., CH); and n is 1. In some embodiments, the compound of Formula (I) is Compound 154, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., N(CH³)); M is C(R²) (e.g., CH); P is N; R⁷ is absent; D is N(R^{5c}) (e.g., NH); E is N; and F is C(R^{5a})(e.g., CH). In some embodiments, the compound of Formula (I) is Compound 155, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g, N(CH³)); M is C(R²) (e.g., CH); P is N; R⁷ is halo (e.g., F); D is N(R^{5c}) (e.g., NH); E is N; F is C(R^{5a})(e.g., CH); and n is 1. In some embodiments, the compound of Formula (I) is Compound 156, or a pharmaceutically acceptable salt, solvate, hydrate, tautomer, or stereoisomer thereof.

In some embodiments, for Formula (I), A is monocyclic heterocyclyl (e.g., 2-methypiperidine); B is monocyclic heteroaryl (e.g., pyrazolyl); L is —N(R³)— (e.g., N(CH³)); M and P are each independently C(R²) (e.g., CH); R⁷ is absent; D is N(R^{5c}) (e.g., NH); E and F are independently C(R^{5a})(e.g., CH). In some embodiments, the compound of Formula (I) is Compound 157, Compound 158, or a pharmaceutically acceptable salt, solvate, hydrate, tautomer, or stereoisomer thereof.

In some embodiments, for Formula (I), A is monocyclic heterocyclyl (e.g., 2-methypiperidine); B is monocyclic heteroaryl (e.g., pyrazolyl); L is —N(R³)— (e.g., —N(CH³)—); M and P are each independently C(R²) (e.g., CH); R⁷ is absent; D is N(R^{5c}) (e.g., NH); E is N; and F is C(R^{5a})(e.g., CH). In some embodiments, the compound of Formula (I) is Compound 159, Compound 160, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g, N(CH³)); M and P are each independently C(R²) (e.g., CH); D is N(R^{5c}) (e.g., NH); F is N; and E is C(R^{5a})(e.g., CH). In some embodiments, the compound of Formula (I) is Compound 163, Compound 170, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyridinonyl); L is —N(R³)— (e.g., N(CH³)); M and P are each independently C(R²) (e.g., CH); D is N(R^{5c}) (e.g., NH); F is S; and E is C(R^{5a})(e.g., CH). In some embodiments, the compound of Formula (I) is Compound 174, Compound 178, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., methylpyrazolyll); L is —N(R³)— (e.g., —N(CH³)—); M and P are each independently C(R²) (e.g., CH); D is N(R^{5c}) (e.g., NH); F is S; and E is C(R^{5a})(e.g., CH). In some embodiments, the compound of Formula (I) is Compound 175, Compound 176, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 2-methoxypyridinyl); L is —N(R³)— (e.g., —N(CH³)—); M and P are each independently C(R²) (e.g., CH); D is N(R^{5c}) (e.g., NH); F is S; and E is C(R^{5a})(e.g., CH). In some embodiments, the compound of Formula (I) is Compound 177 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrimidonyl); L is —N(R³)— (e.g., N(CH³)); M and P are each independently C(R²) (e.g., CH); D is N(R^{5c}) (e.g., NH); F is S; and E is C(R^{5a})(e.g., CH). In some embodiments, the compound of Formula (I) is Compound 179 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyridinonyl); L is —N(R³)— (e.g., N(CH³)); M and P are each independently C(R²) (e.g., CH); D is N(R^{5c}) (e.g., NH); F is S; and E is C(R^{5a})(e.g., CH). In some embodiments, the compound of Formula (I) is Compound 180 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —O—; M and P are each independently C(R²) (e.g., CH); D is N(R^{5c}) (e.g., NH); F is S; and E is C(R^{5a})(e.g., CH). In some embodiments, the compound of Formula (I) is Compound 181 or a pharmaceutically acceptable salt, solvate, hydrate, tautomer, or stereoisomer thereof.

In some embodiments, for Formula (I), A is monocyclic heterocyclyl (e.g., 2,2-dimethylpiperidinyl); B is monocyclic heteroaryl (e.g., pyrazolyl); L is —O—; M and P are each independently C(R²) (e.g., CH); D is N(R^{5c}) (e.g., NH); F is S; and E is C(R^{5a})(e.g., CH). In some embodiments, the compound of Formula (I) is Compound 182 or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —O—; M and P are each independently C(R²) (e.g., CH); D is N(R^{5c}) (e.g., NH); F is S; and E is C(R^{5a})(e.g., CH). In some embodiments, the compound of Formula (I) is Compound 183 or a pharmaceutically acceptable salt, solvate, hydrate, tautomer, or stereoisomer thereof.

In some embodiments, for Formula (I), A is monocyclic heterocyclyl (e.g., piperazinyl); B is monocyclic heteroaryl (e.g., pyrazolyl); L is absent; M and P are each independently C(R²) (e.g., CH); F is N; D is S; and E is C(R^{5a})(e.g., CH). In some embodiments, the compound of Formula (I) is Compound 185 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R³)— (e.g., —N(CH³)—); M and P are each independently C(R²) (e.g., CH); F is N; D is S; and E is C(R^{5a})(e.g., CH). In some embodiments, the compound of Formula (I) is Compound 186 or a pharmaceutically acceptable salt, solvate, hydrate, tautomer, or stereoisomer thereof.

In some embodiments, for Formula (I), A is bicyclic heterocyclyl (e.g., 2,6-diazaspiro[3.5]nonane); B is monocyclic heteroaryl (e.g., pyrazolyl); L is absent; M and P are each independently C(R²) (e.g., CH); F is N; D is S; and E is C(R^{5a})(e.g., CH). In some embodiments, the compound of Formula (I) is Compound 187, Compound 189, or a pharmaceutically acceptable salt, solvate, hydrate, tautomer, or stereoisomer thereof.

In some embodiments, for Formula (I), A is bicyclic heterocyclyl (e.g., 2,7-diazaspiro[3.5]nonane); B is monocyclic heteroaryl (e.g., pyrazolyl); L is absent; M and P are each independently C(R²) (e.g., CH); F is N; D is S; and E is C(R^{5a})(e.g., CH). In some embodiments, the compound of Formula (I) is Compound 188 or a pharmaceutically acceptable salt, solvate, hydrate, tautomer, or stereoisomer thereof.

In some embodiments, for Formula (I), A is bicyclic heterocyclyl (e.g., 2,8-diazaspiro[4.5]decanyl); B is monocyclic heteroaryl (e.g., pyrazolyl); L is absent; M and P are each independently C(R²) (e.g., CH); F is N; D is S; and E is C(R^{5a})(e.g., CH). In some embodiments, the compound of Formula (I) is Compound 190, Compound 192, or a pharmaceutically acceptable salt, solvate, hydrate, tautomer, or stereoisomer thereof.

In some embodiments, for Formula (I), A is bicyclic heterocyclyl (e.g., 2,7-diazaspiro[4.5]decanyl); B is monocyclic heteroaryl (e.g., pyrazolyl); L is absent; M and P are each independently C(R²) (e.g., CH); F is N; D is S; and E is C(R^{5a})(e.g., CH). In some embodiments, the compound of Formula (I) is Compound 191, Compound 193, or a pharmaceutically acceptable salt, solvate, hydrate, tautomer, or stereoisomer thereof.

In some embodiments, for Formula (I), A is monocyclic heterocyclyl (e.g., azetidinyl); B is monocyclic heteroaryl (e.g., pyrazolyl); L is —N(R³)— (e.g., N(CH³)); M and P are each independently C(R²) (e.g., CH); F is N; D is S; and E is C(R^{5a})(e.g., CH). In some embodiments, the compound of Formula (I) is Compound 196 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —O—; M and P are each independently C(R²) (e.g., CH); F is N; D is S; and E is C(R^{5a})(e.g., CH). In some embodiments, the compound of Formula (I) is Compound 199 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —S—; M and P are each independently C(R²) (e.g., CH); F is N; D is S; and E is C(R^{5a})(e.g., CH). In some embodiments, the compound of Formula (I) is Compound 200 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 2-methyl-1,3-oxazolyl); L is —N(R³)— (e.g., N(CH³)); M and P are each independently C(R²) (e.g., CH); D is N; F is S; and E is C(R^{5a})(e.g., CH). In some embodiments, the compound of Formula (I) is Compound 201 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., NH); M and P are each independently C(R²) (e.g., CH); F is N; D is S; and E is C(R^{5a})(e.g., CH). In some embodiments, the compound of Formula (I) is Compound 202, Compound 391, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —N(CH₂CH₃)—); M and P are each independently C(R²) (e.g., CH); F is N; D is S; and E is C(R^{5a})(e.g., CH). In some embodiments, the compound of Formula (I) is Compound 203 or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is absent; M and P are each independently C(R²) (e.g., CH); F is N; D is S; and E is C(R^{5a})(e.g., CH). In some embodiments, the compound of Formula (I) is Compound 206 or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —O—; M and P are each independently C(R²) (e.g., CH); F is N; D is S; and E is C(R^{5a})(e.g., CH). In some embodiments, the compound of Formula (I) is Compound 209 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —N(CD₃)—); M and P are each independently C(R²) (e.g., CH); F is N; D is S; and E is C(R^{5a})(e.g., CH). In some embodiments, the compound of Formula (I) is Compound 210 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyridazinonyl); L is —N(R³)— (e.g., —N(CH₃)—); M and P are each independently C(R²) (e.g., CH); D is N; F is S; and E is C(R^{5a})(e.g., CH). In some embodiments, the compound of Formula (I) is Compound 211 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrimidinonyl); L is —N(R³)— (e.g., —N(CH₃)—); M and P are each independently C(R²) (e.g., CH); D is N; F is S; and E is C(R^{5a})(e.g., CH). In some embodiments, the compound of Formula (I) is Compound 212 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —N(CH₃)—); M and P are each independently C(R²) (e.g., CH); F is N; D is S; and E is C(R^{5a})(e.g., CH). In some embodiments, the compound of Formula (I) is Compound 213, Compound 390, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —N(R³)— (e.g., —NH—); M and P are each independently C(R²) (e.g., CH); F is N; D is S; and E is C(R^{5a})(e.g., CH). In some embodiments, the compound of Formula (I) is Compound 214 or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., —NH—); M and P are each independently C(R²) (e.g., CH); F is N; D is S; and E is C(R^{5a})(e.g., CH). In some embodiments, the compound of Formula (I) is Compound 215 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —O—; M and P are each independently C(R²) (e.g., CH); R⁷ is halo (e.g. F); D is N(R^{5c}) (e.g., NH); E is N; F is C(R^{5a})(e.g., CH); and n is 1. In some embodiments, the compound of Formula (I) is Compound 216 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —N(CH₃)—); M and P are each independently C(R²) (e.g., CH); R⁷ is absent; D is N(R^{5c}) (e.g., NH); E is N; and F is C(R^{5a})(e.g., CH). In some embodiments, the compound of Formula (I) is Compound 217 or a pharmaceutically acceptable salt, solvate, hydrate, tautomer, or stereoisomer thereof.

In some embodiments, for Formula (I), A is bicyclic heterocyclyl (e.g., 2,6-diazaspiro[3.5]nonanyl); B is monocyclic heteroaryl (e.g., pyrazolyl); L is —N(R³)— (e.g., —NCH₃—); M and P are each independently C(R²) (e.g., CH); R⁷ is absent; D is N(R^{5c}) (e.g., NH); E is N; and F is C(R^{5a})(e.g., CH). In some embodiments, the compound of Formula (I) is Compound 218 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —O—; M and P are each independently C(R²) (e.g., CH); R⁷ is absent; D is N(R^{5c}) (e.g., NH); E is N; and F is C(R^{5a})(e.g., CH). In some embodiments, the compound of Formula (I) is Compound 219 or a pharmaceutically acceptable salt, solvate, hydrate, tautomer, or stereoisomer thereof.

In some embodiments, for Formula (I), A is monocyclic heterocyclyl (e.g., 2,2-dimethylpiperidinyl); B is monocyclic heteroaryl (e.g., pyrazolyl); L is —O—; M and P are each independently C(R²) (e.g., CH); R⁷ is absent; D is N(R^{5c}) (e.g., NH); E is N; and F is C(R^{5a})(e.g., CH). In some embodiments, the compound of Formula (I) is Compound 220 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —S—; M and P are each independently C(R²) (e.g., CH); R⁷ is absent; D is N(R^{5c}) (e.g., NH); E is N; and F is C(R^{5a})(e.g., CH). In some embodiments, the compound of Formula (I) is Compound 221 or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —N(R³)— (e.g., —N(CH₃)—); M and P are each independently C(R²) (e.g., CH); F is N; D is S; and E is C(R^{5a})(e.g., CH). In some embodiments, the compound of Formula (I) is Compound 222 or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —N(R³)— (e.g., —N(CD₃)—); M and P are each independently C(R²) (e.g., CH); F is N; D is S; and E is C(R^{5a})(e.g., CH). In some embodiments, the compound of Formula (I) is Compound 223 or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —N(R³)— (e.g., —N(CH₂CH₃)—); M and P are each independently C(R²) (e.g., CH); F is N; D is S; and E is C(R^{5a})(e.g., CH). In some embodiments, the compound of Formula (I) is Compound 224 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R³)— (e.g., —N(CH₂CH₃)—); M and P are each independently C(R²) (e.g., CH); F is N; D is S; and E is C(R^{5a})(e.g., CH). In some embodiments, the compound of Formula (I) is Compound 225 or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., —N(CH₃)—); M and P are each independently C(R²) (e.g., CH); F is N; D is S; and E is C(R^{5a})(e.g., CH). In some embodiments, the compound of Formula (I) is Compound 226 or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., —N(CD₃)—); M and P are each independently C(R²) (e.g., CH); F is N; D is S; and E is C(R^{5a})(e.g., CH). In some embodiments, the compound of Formula (I) is Compound 227 or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., —N(CH₂CH₃)—); M and P are each independently C(R²) (e.g., CH); F is N; D is S; and E is C(R^{5a})(e.g., CH). In some embodiments, the compound of Formula (I) is Compound 228 or a pharmaceutically acceptable salt, solvate, hydrate, tautomer, or stereoisomer thereof.

In some embodiments, for Formula (I), A is bicyclic heterocyclyl (e.g., 2,7-diazaspiro[3.5]nonanyl); B is monocyclic heteroaryl (e.g., pyrazolyl); L is absent; M and P are each independently C(R²) (e.g., CH); R⁷ is absent; D is N(R^{5c}) (e.g., NH); E is N; and F is C(R^{5a})(e.g., CH). In some embodiments, the compound of Formula (I) is Compound 229 or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —O—; M and P are each independently C(R²) (e.g., CH); R⁷ is absent; D is N(R^{5c}) (e.g., NH); E is N; and F is C(R^{5a})(e.g., CH). In some embodiments, the compound of Formula (I) is Compound 230 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R³)— (e.g., —N(CD₃)—); M and P are each independently C(R²) (e.g., CH); R⁷ is absent; F is N; D is S; and E is C(R^{5a})(e.g., CH). In some embodiments, the compound of Formula (I) is Compound 231 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., methylpyrazolyl); L is —O—; M and P are each independently C(R²) (e.g., CH); R⁷ is absent; D is N(R^{5c}) (e.g., NH); E is N; and F is C(R^{5a})(e.g., CH). In some embodiments, the compound of Formula (I) is Compound 232 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 3-methoxypyridazinyl); L is —N(R³)— (e.g., —N(CH₃)—); M and P are each independently C(R²) (e.g., CH); R⁷ is absent; D is N; F is S; and E is C(R^{5a})(e.g., CH). In some embodiments, the compound of Formula (I) is Compound 233 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 3-hydroxypyridazinyl); L is —N(R³)— (e.g., —N(CH₃)—); M and P are each independently C(R²) (e.g., CH); R⁷ is absent; D is N; F is S; and E is C(R^{5a})(e.g., CH). In some embodiments, the compound of Formula (I) is Compound 234 or a pharmaceutically acceptable salt, solvate, hydrate, tautomer, or stereoisomer thereof.

In some embodiments, for Formula (I), A is monocyclic heterocyclyl (e.g., azetidinyl); B is monocyclic heteroaryl (e.g., pyrazolyl); L is —N(R³)— (e.g., N(CH³)); M and P are each independently C(R²) (e.g., CH); D is N(R^{5c}) (e.g., NH); E is N; and F is C(R^{5a})(e.g., CH). In some embodiments, the compound of Formula (I) is Compound 237 or a pharmaceutically acceptable salt, solvate, hydrate, tautomer, or stereoisomer thereof.

In some embodiments, for Formula (I), A is bicyclic heterocyclyl (e.g., 2,7-diazaspiro[3.5]nonanyl); B is monocyclic heteroaryl (e.g., pyrazolyl); L is absent; M and P are each independently C(R²) (e.g., CH); D is N(R^{5c},) (e.g., NH); E is N; and F is C(R^{5a})(e.g., CH). In some embodiments, the compound of Formula (I) is Compound 238 or a pharmaceutically acceptable salt, solvate, hydrate, tautomer, or stereoisomer thereof.

In some embodiments, for Formula (I), A is bicyclic heterocyclyl (e.g., 2,8-diazaspiro[4.5]decanyl); B is monocyclic heteroaryl (e.g., pyrazolyl); L is absent; M and P are each independently C(R²) (e.g., CH); F is N(R^{5c},) (e.g., NH); E is N; and D is C(R^{5a})(e.g., CH). In some embodiments, the compound of Formula (I) is Compound 239, Compound 240, or a pharmaceutically acceptable salt, solvate, hydrate, tautomer, or stereoisomer thereof.

In some embodiments, for Formula (I), A is monocyclic heterocyclyl (e.g., piperazinyl); B is monocyclic heteroaryl (e.g., pyrazolyl); L is absent; M and P are each independently C(R²) (e.g., CH); D is N(R^{5c}) (e.g., NH); E is N; and F is C(R^{5a})(e.g., CH). In some embodiments, the compound of Formula (I) is Compound 241 or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., methylpyrazolyl); L is —N(R³)— (e.g., —N(CH³)—); M and P are each independently C(R²) (e.g., CH); D is N(R^{5c},) (e.g., NH); E is N; and F is C(R^{5a})(e.g., CH). In some embodiments, the compound of Formula (I) is Compound 242 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., methylpyrazolyl); L is —N(R³)— (e.g., —N(CH³)—); M and P are each independently C(R²) (e.g., CH); D is N(R^{5c}) (e.g., NH); E is N; and F is C(R^{5a})(e.g., CH). In some embodiments, the compound of Formula (I) is Compound 243 or a pharmaceutically acceptable salt, solvate, hydrate, tautomer, or stereoisomer thereof.

In some embodiments, for Formula (I), A is bicyclic heterocyclyl (e.g., 2,6-diazaspiro[3.5]nonanyl); B is monocyclic heteroaryl (e.g., pyrazolyl); L is absent; M and P are each independently C(R²) (e.g., CH); F is N(R^{5c},) (e.g., NH); E is N; and D is C(R^{5a})(e.g., CH). In some embodiments, the compound of Formula (I) is Compound 251 or a pharmaceutically acceptable salt, solvate, hydrate, tautomer, or stereoisomer thereof.

In some embodiments, for Formula (I), A is monocyclic heterocyclyl (e.g., 2,7-diazaspiro[4.5]decanyl); B is monocyclic heteroaryl (e.g., pyrazolyl); L is absent; M and P are each independently C(R²) (e.g., CH); D is N(R^{5c},) (e.g., NH); E is N; and F is C(R^{5a})(e.g., CH). In some embodiments, the compound of Formula (I) is Compound 252, Compound 253, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —N(R³)— (e.g., —NH—); M and P are each independently C(R²) (e.g., CH); D is N(R^{5c}) (e.g., NH); E is N; and F is C(R^{5a})(e.g., CH). In some embodiments, the compound of Formula (I) is Compound 263, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —N(R³)— (e.g., —N(CH₃)—); M and P are each independently C(R²) (e.g., CH); F is N(R^{5c}) (e.g., NH); E is N; and D is C(R^{5a})(e.g., CH). In some embodiments, the compound of Formula (I) is Compound 264, Compound 273or a pharmaceutically acceptable salt, solvate, hydrate, tautomer, or stereoisomer thereof.

In some embodiments, for Formula (I), A is bicyclic heterocyclyl (e.g., 1,6-diazaspiro[3.4]octanyl); B is monocyclic heteroaryl (e.g., pyrazolyl); L is —N(R³)— (e.g., —N(CH₃)—); M and P are each independently C(R²) (e.g., CH); R⁷ is absent; F is N; D is S; and E is C(R^{5a})(e.g., CH). In some embodiments, the compound of Formula (I) is Compound 265 or a pharmaceutically acceptable salt, solvate, hydrate, tautomer, or stereoisomer thereof.

In some embodiments, for Formula (I), A is bicyclic heterocyclyl (e.g., 1,7-diazaspiro[3.5]nonanyl); B is monocyclic heteroaryl (e.g., pyrazolyl); L is —N(R³)— (e.g., —N(CH₃)—); M and P are each independently C(R²) (e.g., CH); R⁷ is absent; F is N; D is S; and E is C(R^{5a})(e.g., CH). In some embodiments, the compound of Formula (I) is Compound 266 or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —N(R³)— (e.g., —N(CD₃)—); M and P are each independently C(R²) (e.g., CH); D is N(R^{5c}) (e.g., NH); E is N; and F is C(R^{5a})(e.g., CH). In some embodiments, the compound of Formula (I) is Compound 267, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —N(R³)— (e.g., —N(CH₂CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 274, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R³)— (e.g., —NH—); M and P are each independently C(R²) (e.g., CH); D is N(R^{5c}) (e.g., NH); E is N; and F is C(R^{5a})(e.g., CH). In some embodiments, the compound of Formula (I) is Compound 275, or a pharmaceutically acceptable salt, solvate, hydrate, tautomer, or stereoisomer thereof.

In some embodiments, for Formula (I), A is bicyclic heterocyclyl (e.g., 1,6-diazaspiro[3.5]nonanyl); B is monocyclic heteroaryl (e.g., pyrazolyl); L is —N(R³)— (e.g., —N(CH₃)—); M and P are each independently C(R²) (e.g., CH); R⁷ is absent; F is N; D is S; and E is C(R^{5a})(e.g., CH). In some embodiments, the compound of Formula (I) is Compound 276 or a pharmaceutically acceptable salt, solvate, hydrate, tautomer, or stereoisomer thereof.

In some embodiments, for Formula (I), A is bicyclic heterocyclyl (e.g., 1,6-diazaspiro[3.4]octanyl); B is monocyclic heteroaryl (e.g., pyrazolyl); L is —N(R³)— (e.g., —N(CH₃)—); M and P are each independently C(R²) (e.g., CH); R⁷ is absent; F is N; D is S; and E is C(R^{5a})(e.g., CH). In some embodiments, the compound of Formula (I) is Compound 277 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —NH—)); M and P are each C(R²) (e.g., CH); R⁷ is absent; D is N(R^{5c}) (e.g., NH); E is N; and F is C(R^{5a})(e.g., CH). In some embodiments, the compound of Formula (I) is Compound 278, Compound 395, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyridinonyl); L is —N(R³)— (e.g., N(CH³)); M and P are each independently C(R²) (e.g., CH); D is S; F is N; and E is C(R^{5a})(e.g., CH). In some embodiments, the compound of Formula (I) is Compound 279, Compound 281, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 2-methoxypyridinyl); L is —N(R³)— (e.g., N(CH³)); M and P are each independently C(R²) (e.g., CH); D is S; F is N; and E is C(R^{5a})(e.g., CH). In some embodiments, the compound of Formula (I) is Compound 280 or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 2-methoxypyridinyl); L is —N(R³)— (e.g., aminocyclopropyl); M and P are each independently C(R²) (e.g., CH); D is S; F is N; and E is C(R^{5a})(e.g., CH). In some embodiments, the compound of Formula (I) is Compound 283 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 2-methoxypyridinyl); L is —N(R³)— (e.g., aminocyclopropyl); M and P are each independently C(R²) (e.g., CH); D is S; F is N; and E is C(R^{5a})(e.g., CH). In some embodiments, the compound of Formula (I) is Compound 284 or a pharmaceutically acceptable salt, solvate, hydrate, tautomer, or stereoisomer thereof.

In some embodiments, for Formula (I), A is bicyclic heterocyclyl (e.g., 1-methyl-1,6-diazaspiro[3.4]octanyl); B is monocyclic heteroaryl (e.g., 2-methoxypyridinyl); L is —N(R³)— (e.g., aminocyclopropyl); M and P are each independently C(R²) (e.g., CH); D is S; F is N; and E is C(R^{5a})(e.g., CH). In some embodiments, the compound of Formula (I) is Compound 287 or a pharmaceutically acceptable salt, solvate, hydrate, tautomer, or stereoisomer thereof.

In some embodiments, for Formula (I), A is bicyclic heterocyclyl (e.g., 6-methyl-1,6-diazaspiro[3.5]nonanyl); B is monocyclic heteroaryl (e.g., 2-methoxypyridinyl); L is —N(R³)— (e.g., aminocyclopropyl); M and P are each independently C(R²) (e.g., CH); D is S; F is N; and E is C(R^{5a})(e.g., CH). In some embodiments, the compound of Formula (I) is Compound 288 or a pharmaceutically acceptable salt, solvate, hydrate, tautomer, or stereoisomer thereof.

In some embodiments, for Formula (I), A is bicyclic heterocyclyl (e.g., 7-methyl-1,7-diazaspiro[3.5]nonanyl); B is monocyclic heteroaryl (e.g., 2-methoxypyridinyl); L is —N(R³)— (e.g., aminocyclopropyl); M and P are each independently C(R²) (e.g., CH); D is S; F is N; and E is C(R^{5a})(e.g., CH). In some embodiments, the compound of Formula (I) is Compound 289 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., methylpyrazolyl); L is —N(R³)— (e.g., N(CH³)); M is C(R²) (e.g., CH); P is N; R⁷ is halo (e.g., F); D is N(R^{5c}) (e.g., NH); E is N; F is C(R^{5a})(e.g., CH); and n is 1. In some embodiments, the compound of Formula (I) is Compound 290, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., methylpyrazolyl); L is —N(R³)— (e.g., N(CH³)); M and P are each independently C(R²) (e.g., CH); R⁷ is halo (e.g., F); D is N(R^{5c}) (e.g., NH); E is N; F is C(R^{5a})(e.g., CH); and n is 1. In some embodiments, the compound of Formula (I) is Compound 291, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R³)— (e.g., —N(CD₃)—); M and P are each independently C(R²) (e.g., CH); D is N(R^{5c}) (e.g., NH); E is N; and F is C(R^{5a})(e.g., CH). In some embodiments, the compound of Formula (I) is Compound 292 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R³)— (e.g, —N(CH₂CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 293 or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., —NH—); M and P are each independently C(R²) (e.g., CH); D is N(R^{5c}) (e.g., NH); E is N; and F is C(R^{5a})(e.g., CH). In some embodiments, the compound of Formula (I) is Compound 294, Compound 298, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g, —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 295 or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g, —N(CD₃)—); M and P are each independently C(R²) (e.g., CH); D is N(R^{5c}) (e.g., NH); E is N; and F is C(R^{5a})(e.g., CH). In some embodiments, the compound of Formula (I) is Compound 296, Compound 299 or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., —N(CH₂CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 297, Compound 300, or a pharmaceutically acceptable salt, solvate, hydrate, tautomer, or stereoisomer thereof.

In some embodiments, for Formula (I), A is monocyclic heterocyclyl (e.g., pyrrolidinyl); R¹ is N(R^{B}R^{C})— (e.g., —N(C(CH₃)₃)—); B is monocyclic heteroaryl (e.g., pyrazolyl); L is absent; M and P are each independently C(R²) (e.g., CH); D is N(R^{5c}) (e.g., NH); E is N; and F is C(R^{5a})(e.g., CH). In some embodiments, the compound of Formula (I) is Compound 305, Compound 310, or a pharmaceutically acceptable salt, solvate, hydrate, tautomer, or stereoisomer thereof.

In some embodiments, for Formula (I), A is monocyclic heterocyclyl (e.g., pyrrolidinyl); B is monocyclic heteroaryl (e.g., pyrazolyl); L is absent; M and P are each independently C(R²) (e.g., CH); D is N(R^{5c}) (e.g., NH); E is N; and F is C(R^{5a})(e.g., CH). In some embodiments, the compound of Formula (I) is Compound 306, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —O—; M and P are each independently C(R²) (e.g., CH); R⁷ is halo (e.g., F); D is N(R^{5c}) (e.g., NH); E is N; F is C(R^{5a})(e.g., CH); and n is 1. In some embodiments, the compound of Formula (I) is Compound 307, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —O—; M and P are each independently C(R²) (e.g., CH); F is N(R^{5c}) (e.g., NH); E is N; D is C(R^{5a})(e.g., CH). In some embodiments, the compound of Formula (I) is Compound 308, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is N(R³)— (e.g, —N(CH₃)—); M and P are each independently C(R²) (e.g., CH); F is N(R^{5c}) (e.g., NH); E is N; D is C(R^{5a})(e.g., CH). In some embodiments, the compound of Formula (I) is Compound 309, or a pharmaceutically acceptable salt, solvate, hydrate, tautomer, or stereoisomer thereof.

In some embodiments, for Formula (I), A is monocyclic heterocyclyl (e.g., 9-azabicyclo[3.3.1]nonanyl); B is monocyclic heteroaryl (e.g., pyrazolyl); L is —N(R³)— (e.g., N(C₃H₅)—); M and P are each independently C(R²) (e.g., CH); D is S; F is N; and E is C(R^{5a})(e.g., CH). In some embodiments, the compound of Formula (I) is Compound 311 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1,2,3-triazolyl); L is N(R³)— (e.g., —N(CH₃)—); M and P are each independently C(R²) (e.g., CH); D is N(R^{5c}) (e.g., NH); E is N; F is C(R^{5a})(e.g., CH). In some embodiments, the compound of Formula (I) is Compound 312, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —O—; M and P are each independently C(R²) (e.g., CH); D is N(R^{5c}) (e.g., NH); R⁷ is halo (e.g., F); E is N; F is C(R^{5a})(e.g., CH); and n is 1. In some embodiments, the compound of Formula (I) is Compound 313, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 1,2,3-triazolyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 315 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1,2,3-triazolyl); L is —O—; M and P are each independently C(R²) (e.g., CH); D is N(R⁵,) (e.g., NH); E is N; and F is C(R^{5a})(e.g., CH). In some embodiments, the compound of Formula (I) is Compound 316 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1,2,3-triazolyl); L is —N(R³)— (e.g., —N(CH₃)—); M and P are each independently C(R²) (e.g., CH); F is N(R^{5c}) (e.g., NH); E is N; and D is C(R^{5a})(e.g., CH). In some embodiments, the compound of Formula (I) is Compound 317 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1,2,3-triazolyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 318 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1,2,3-triazolyl); L is —O—; M and P are each independently C(R²) (e.g., CH); R⁷ is halo (e.g., F); D is N(R^{5c}) (e.g., NH); E is N; and F is C(R^{5a})(e.g., CH); and n is 1. In some embodiments, the compound of Formula (I) is Compound 319 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 2-ethyltriazolyl); L is —N(R³)— (e.g, —N(CH₃)—); M and P are each independently C(R²) (e.g., CH); F is N(R^{5c}) (e.g., NH); E is N; and F is C(R^{5a})(e.g., CH). In some embodiments, the compound of Formula (I) is Compound 320 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1,2,3-triazolyl); L is —O—; M and P are each independently C(R²) (e.g., CH); D is N(R^{5c}) (e.g., NH); E is N; and F is C(R^{5a})(e.g., CH). In some embodiments, the compound of Formula (I) is Compound 321 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1,2,3-triazolyl); L is —N(R³)— (e.g., —NH—); M and P are each independently C(R²) (e.g., CH); D is N(R^{5c}) (e.g., NH); E is N; and F is C(R^{5a})(e.g., CH). In some embodiments, the compound of Formula (I) is Compound 322 or a pharmaceutically acceptable salt, solvate, hydrate, tautomer, or stereoisomer thereof.

In some embodiments, for Formula (I), A is monocyclic heterocyclyl (e.g., 8-azabicyclo[3.2.1]octanyl); B is monocyclic heteroaryl (e.g., 1,2,3-triazolyl); L is —O—; M and P are each independently C(R²) (e.g., CH); D is N(R^{5c}) (e.g., NH); E is N; and F is C(R^{5a})(e.g., CH). In some embodiments, the compound of Formula (I) is Compound 323 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1,2,3-triazolyl); L is —N(R³)— (e.g., —NH—); M and P are each independently C(R²) (e.g., CH); F is N(R^{5c}) (e.g., NH); E is N; and D is C(R^{5a})(e.g., CH). In some embodiments, the compound of Formula (I) is Compound 324 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1,2,3-triazolyl); L is —O—; M and P are each independently C(R²) (e.g., CH); D is N(R^{5c},) (e.g., NH); E is N; and F is C(R^{5a})(e.g., CH). In some embodiments, the compound of Formula (I) is Compound 325 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., methylpyrazolyl); L is —N(R³)— (e.g., —N(CH₃)—); M and P are each independently C(R²) (e.g., CH); D is N(R^{5c}) (e.g., NH); R⁷ is halo (e.g. F); E is N; and F is C(R^{5a})(e.g., CH); and n is 1. In some embodiments, the compound of Formula (I) is Compound 326 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —O—; M and P are each independently C(R²) (e.g., CH); F is N(R^{5c}) (e.g., NH); E is N; and D is C(R^{5a})(e.g., CH). In some embodiments, the compound of Formula (I) is Compound 328, Compound 394, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —N(CH₃)—); M and P are each independently C(R²) (e.g., CH); F is N(R^{5c}) (e.g., NH); E is N; and D is C(R^{5a})(e.g., CH). In some embodiments, the compound of Formula (I) is Compound 329, Compound 393, Compound 475 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 3-methoxypyridazinyl); L is —N(R³)— (e.g., —N(CH₃)); M and P are each independently C(R²) (e.g., CH); D is S; F is N; and E is C(R^{5a})(e.g., CH). In some embodiments, the compound of Formula (I) is Compound 330 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyridazinonyl); L is —N(R³)— (e.g., —N(CH₃)); M and P are each independently C(R²) (e.g., CH); D is S; F is N; and E is C(R^{5a})(e.g., CH). In some embodiments, the compound of Formula (I) is Compound 331 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrimidinonyl); L is —N(R³)— (e.g., —N(CH₃)); M and P are each independently C(R²) (e.g., CH); D is S; F is N; and E is C(R^{5a})(e.g., CH). In some embodiments, the compound of Formula (I) is Compound 332 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyridonyl); L is —N(R³)— (e.g., —N(CH₃)); M and P are each independently C(R²) (e.g., CH); D is S; F is N; and E is C(R^{5a})(e.g., CH). In some embodiments, the compound of Formula (I) is Compound 333 or a pharmaceutically acceptable salt, solvate, hydrate, tautomer, or stereoisomer thereof.

In some embodiments, for Formula (I), A is monocyclic heterocyclyl (e.g., 8-azabicyclo[3.2.1]octanyl); B is monocyclic heteroaryl (e.g., pyrazolyl); L is —O—; M and P are each independently C(R²) (e.g., CH); D is S; F is N; and E is C(R^{5a})(e.g., CH). In some embodiments, the compound of Formula (I) is Compound 334 or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —O—; M and P are each independently C(R²) (e.g., CH); D is S; F is N; and E is C(R^{5a})(e.g., CH). In some embodiments, the compound of Formula (I) is Compound 335 or a pharmaceutically acceptable salt, solvate, hydrate, tautomer, or stereoisomer thereof.

In some embodiments, for Formula (I), A is monocyclic heterocyclyl (e.g., pyrrolidinyl); R¹ is N(R^{B}R^{C})— (e.g., —N(C(CH₃)₃)—); B is monocyclic heteroaryl (e.g., pyrazolyl); L is absent; M and P are each independently C(R²) (e.g., CH); D is S; F is N; and E is C(R^{5a})(e.g., CH). In some embodiments, the compound of Formula (I) is Compound 336, Compound 345, or a pharmaceutically acceptable salt, solvate, hydrate, tautomer, or stereoisomer thereof.

In some embodiments, for Formula (I), A is monocyclic heterocyclyl (e.g., pyrrolidinyl); R¹ is —N(R^{B}R^{C}) (e.g., —NH(C₃H₅)); B is monocyclic heteroaryl (e.g., pyrazolyl); L is absent; M and P are each independently C(R²) (e.g., CH); D is S; F is N; and E is C(R^{5a})(e.g., CH). In some embodiments, the compound of Formula (I) is Compound 337, Compound 346, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —N(CH₃)); M and P are each independently C(R²) (e.g., CH); R⁷ is halo (e.g. F); D is S; F is N; E is C(R^{5a})(e.g., CH); and n is 1. In some embodiments, the compound of Formula (I) is Compound 339, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1-methylpyrazolyl); L is —N(R³)— (e.g., —N(CH₃)); M and P are each independently C(R²) (e.g., CH); D is S; F is N; E is C(R^{5a})(e.g., CH). In some embodiments, the compound of Formula (I) is Compound 340, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., methylpyrazolyl); L is —N(R³)— (e.g., —N(CH₃)); M and P are each independently C(R²) (e.g., CH); D is S; F is N; E is C(R^{5a})(e.g., CH). In some embodiments, the compound of Formula (I) is Compound 341, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., methyloxazolyl); L is —N(R³)— (e.g., —N(CH₃)); M and P are each independently C(R²) (e.g., CH); D is S; F is N; E is C(R^{5a})(e.g., CH). In some embodiments, the compound of Formula (I) is Compound 342, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —NH—)); M and P are each independently C(R²) (e.g., CH); D is S; F is N; E is C(R^{5a})(e.g., CH). In some embodiments, the compound of Formula (I) is Compound 343, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —NCH₃)); M and P are each independently C(R²) (e.g., CH); D is S; F is N; E is C(R^{5a})(e.g., CH). In some embodiments, the compound of Formula (I) is Compound 344, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., methylthiazolyl); L is —O—; M and P are each independently C(R²) (e.g., CH); D is S; F is N; E is C(R^{5a})(e.g., CH. In some embodiments, the compound of Formula (I) is Compound 348, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methylpyridinyl); L is —O—; M and P are each independently C(R²) (e.g., CH); R⁷ is halo (e.g. F); D is S; F is N; E is C(R^{5a})(e.g., CH); and n is 1. In some embodiments, the compound of Formula (I) is Compound 349, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 3-methylpyridazinyl); L is —O—; M and P are each independently C(R²) (e.g., CH); D is S; F is N; E is C(R^{5a})(e.g., CH). In some embodiments, the compound of Formula (I) is Compound 352, or a pharmaceutically acceptable salt, solvate, hydrate, tautomer, or stereoisomer thereof.

In some embodiments, for Formula (I), A is bicyclic heterocyclyl (e.g., 3-oxa-9-azabicyclo[3.3.1]nonanyl); B is monocyclic heteroaryl (e.g., pyrazolyl); L is —N(R³)— (e.g., —NH)); M and P are each independently C(R²) (e.g., CH); D is S; F is N; E is C(R^{5a})(e.g., CH). In some embodiments, the compound of Formula (I) is Compound 353, Compound 354, or a pharmaceutically acceptable salt, solvate, hydrate, tautomer, or stereoisomer thereof.

In some embodiments, for Formula (I), A is bicyclic heterocyclyl (e.g., 3-oxa-9-azabicyclo[3.3.1]nonanyl); B is monocyclic heteroaryl (e.g., pyrazolyl); L is —N(R³)— (e.g., —NCH₃)); M and P are each independently C(R²) (e.g., CH); D is S; F is N; E is C(R^{5a})(e.g., CH). In some embodiments, the compound of Formula (I) is Compound 355, Compound 356, or a pharmaceutically acceptable salt, solvate, hydrate, tautomer, or stereoisomer thereof.

In some embodiments, for Formula (I), A is bicyclic heterocyclyl (e.g., 3-oxa-9-azabicyclo[3.3.1]nonanyl); B is monocyclic heteroaryl (e.g., pyrazolyl); L is —O—; M and P are each independently C(R²) (e.g., CH); D is S; F is N; E is C(R^{5a})(e.g., CH). In some embodiments, the compound of Formula (I) is Compound 357, Compound 358, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., imidazolyl); L is —N(R³)— (e.g., —NCH₃)); M and P are each independently C(R²) (e.g., CH); D is S; F is N; E is C(R^{5a})(e.g., CH). In some embodiments, the compound of Formula (I) is Compound 361, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 3-methoxypyridazinyl); L is —O—; M and P are each independently C(R²) (e.g., CH); D is S; F is N; E is C(R^{5a})(e.g., CH). In some embodiments, the compound of Formula (I) is Compound 362, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 3-fluoropyrazolyl); L is —O—; M and P are each independently C(R²) (e.g., CH); D is S; F is N; E is C(R^{5a})(e.g., CH). In some embodiments, the compound of Formula (I) is Compound 363, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methoxypyridinyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 364, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1-methyl-2-pyridonyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 365, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 3-methylpyridazinyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 366, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-hydroxypyridinyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 367, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 4-methoxypyrimidinyl); L is —O—; M and P are each independently C(R²) (e.g., CH); D is S; F is N; and F is C(R^{5a})(e.g., CH). In some embodiments, the compound of Formula (I) is Compound 368, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methylpyridinyl); L is —O—; M and P are each independently C(R²) (e.g., CH); D is N(R^{5c}) (e.g., NH); E is N; and F is C(R^{5a})(e.g., CH). In some embodiments, the compound of Formula (I) is Compound 369, or a pharmaceutically acceptable salt, solvate, hydrate, tautomer, or stereoisomer thereof.

In some embodiments, for Formula (I), A is bicyclic heterocyclyl (e.g., 3,3-difluoro-8-azabicyclo[3.2.1]octanyl); B is monocyclic heteroaryl (e.g., 2-methylpyridinyl); L is —O—; M and P are each independently C(R²) (e.g., CH); D is N(R^{5c}) (e.g., NH); E is N; and F is C(R^{5a})(e.g., CH). In some embodiments, the compound of Formula (I) is Compound 370, or a pharmaceutically acceptable salt, solvate, hydrate, tautomer, or stereoisomer thereof.

In some embodiments, for Formula (I), A is bicyclic heterocyclyl (e.g., 3,3-difluoro-8-azabicyclo[3.2.1]octanyl); B is monocyclic heteroaryl (e.g., 3-fluoropyrazolyl); L is —O—; M and P are each independently C(R²) (e.g., CH); D is S; F is N; and F is C(R^{5a})(e.g., CH). In some embodiments, the compound of Formula (I) is Compound 371, or a pharmaceutically acceptable salt, solvate, hydrate, tautomer, or stereoisomer thereof.

In some embodiments, for Formula (I), A is bicyclic heterocyclyl (e.g., 3,3-difluoro-8-azabicyclo[3.2.1]octanyl); B is monocyclic heteroaryl (e.g., pyrazolyl); L is —O—; M and P are each independently C(R²) (e.g., CH); D is N(R^{5c}) (e.g., NH); E is N; and F is C(R^{5a})(e.g., CH). In some embodiments, the compound of Formula (I) is Compound 373, or a pharmaceutically acceptable salt, solvate, hydrate, tautomer, or stereoisomer thereof.

In some embodiments, for Formula (I), A is bicyclic heterocyclyl (e.g., 3,3-difluoro-8-azabicyclo[3.2.1]octanyl); B is monocyclic heteroaryl (e.g., pyrazolyl); L is —O—; M and P are each independently C(R²) (e.g., CH); D is S; F is N; and F is C(R^{5a})(e.g., CH). In some embodiments, the compound of Formula (I) is Compound 374, or a pharmaceutically acceptable salt, solvate, hydrate, tautomer, or stereoisomer thereof.

In some embodiments, for Formula (I), A is bicyclic heterocyclyl (e.g., 3,3-difluoro-8-azabicyclo[3.2.1]octanyl); B is monocyclic heteroaryl (e.g., pyrazolyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 376, or a pharmaceutically acceptable salt, solvate, hydrate, tautomer, or stereoisomer thereof.

In some embodiments, for Formula (I), A is bicyclic heterocyclyl (e.g., 3,3-difluoro-8-azabicyclo[3.2.1]octanyl); B is monocyclic heteroaryl (e.g., pyrazolyl); L is —O—; M and P are each independently C(R²) (e.g., CH); R⁷ is halo (e.g., F); D is N(R^{5c}) (e.g., NH); E is N; and F is C(R^{5a})(e.g., CH); and n is 1. In some embodiments, the compound of Formula (I) is Compound 377, or a pharmaceutically acceptable salt, solvate, hydrate, tautomer, or stereoisomer thereof.

In some embodiments, for Formula (I), A is bicyclic heterocyclyl (e.g., 3,3-difluoro-8-azabicyclo[3.2.1]octanyl); B is monocyclic heteroaryl (e.g., pyrazolyl); L is —N(R³)— (e.g., —N(CH₃)—); M and P are each independently C(R²) (e.g., CH); R⁷ is halo (e.g., F); D is N(R^{5c}) (e.g., NH); E is N; and F is C(R^{5a})(e.g., CH); and n is 1. In some embodiments, the compound of Formula (I) is Compound 378, or a pharmaceutically acceptable salt, solvate, hydrate, tautomer, or stereoisomer thereof.

In some embodiments, for Formula (I), A is bicyclic heterocyclyl (e.g., 3,3-difluoro-8-azabicyclo[3.2.1]octanyl); B is monocyclic heteroaryl (e.g., pyrazolyl); L is —N(R³)— (e.g., —N(CH₃)—); M and P are each independently C(R²) (e.g., CH); D is S; F is N; and F is C(R^{5a})(e.g., CH). In some embodiments, the compound of Formula (I) is Compound 379, or a pharmaceutically acceptable salt, solvate, hydrate, tautomer, or stereoisomer thereof.

In some embodiments, for Formula (I), A is bicyclic heterocyclyl (e.g., pyrrolidinyl); R¹ is N(R^{B}R^{C})— (e.g., —N(C(CH₃)₃—)); B is monocyclic heteroaryl (e.g., pyrazolyl); L is absent; M and P are each independently C(R²) (e.g., CH); F is N(R^{5c}) (e.g., NH); E is N; and D is C(R^{5a})(e.g., CH). In some embodiments, the compound of Formula (I) is Compound 380 or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1-methylpyridinyl); L is —O—; M and P are each independently C(R²) (e.g., CH); D is N(R^{5c}) (e.g., NH); E is N; and F is C(R^{5a})(e.g., CH). In some embodiments, the compound of Formula (I) is Compound 381, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1-hydroxypyridinyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 382, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 3-methylpyridazinyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 383, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., N-methylpyridonyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 384, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methoxypyridinyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 385, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1-methylpyrazolyl); L is —N(R³)— (e.g., —N(CH₃)—); M and P are each independently C(R²) (e.g., CH); D is N(R^{5c}) (e.g., NH); E is N; and F is C(R^{5a})(e.g., CH). In some embodiments, the compound of Formula (I) is Compound 386, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —N(C₃H₆)—); M and P are each independently C(R²) (e.g., CH); D is S; F is N; and E is C(R^{5a})(e.g., CH). In some embodiments, the compound of Formula (I) is Compound 389, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —O—; M and P are each independently C(R²) (e.g., CH); R⁷ is halo (e.g. F); D is N(R^{5c}) (e.g., NH); E is N; F is C(R^{5a})(e.g., CH); and n is 1. In some embodiments, the compound of Formula (I) is Compound 404, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., N(CH³)); M and P are each C(R²) (e.g., CH); R⁷ is absent; F is N(R^{5c}) (e.g., NH); E is N; and D is C(R^{5a})(e.g., CH). In some embodiments, the compound of Formula (I) is Compound 409 or a pharmaceutically acceptable salt, solvate, hydrate, tautomer, or stereoisomer thereof.

In some embodiments, for Formula (I), A is monocyclic heterocyclyl (e.g., pyrrolidinyl); R¹ is heteroalkyl (e.g., —CH₂NHCH₃); B is monocyclic heteroaryl (e.g., pyrazolyl); L is absent; M is N; P is C(R²) (e.g., CH); D is S; F is N; and E is C(R^{5a})(e.g., CH). In some embodiments, the compound of Formula (I) is Compound 411 or a pharmaceutically acceptable salt, solvate, hydrate, tautomer, or stereoisomer thereof.

In some embodiments, for Formula (I), A is monocyclic heterocyclyl (e.g., pyrrolidinyl); R¹ is heteroalkyl (e.g., —NHCH₃); B is monocyclic heteroaryl (e.g., pyrazolyl); L is absent; M is N; P is C(R²) (e.g., CH); D is S; F is N; and E is C(R^{5a})(e.g., CH). In some embodiments, the compound of Formula (I) is Compound 412 or a pharmaceutically acceptable salt, solvate, hydrate, tautomer, or stereoisomer thereof.

In some embodiments, for Formula (I), A is monocyclic heterocyclyl (e.g., pyrrolidinyl); R¹ is heteroalkyl (e.g., —N(CH₃)₂); B is monocyclic heteroaryl (e.g., pyrazolyl); L is absent; M is N; P is C(R²) (e.g., CH); D is S; F is N; and E is C(R^{5a})(e.g., CH). In some embodiments, the compound of Formula (I) is Compound 413 or a pharmaceutically acceptable salt, solvate, hydrate, tautomer, or stereoisomer thereof.

In some embodiments, for Formula (I), A is monocyclic heterocyclyl (e.g., piperidinyl); R¹ is heterocyclyl (e.g., cyclopropyl); B is monocyclic heteroaryl (e.g., pyrazolyl); L is absent; M is N; P is C(R²) (e.g., CH); D is S; F is N; and E is C(R^{5a})(e.g., CH). In some embodiments, the compound of Formula (I) is Compound 414, Compound 451, or a pharmaceutically acceptable salt, solvate, hydrate, tautomer, or stereoisomer thereof.

In some embodiments, for Formula (I), A is bicyclic heterocyclyl (e.g., 3-oxa-9-azabicyclo[3.3.1]nonanyl); B is monocyclic heteroaryl (e.g., pyrazolyl); L is —N(R³)— (e.g., —NCH₃)); M and P are each independently C(R²) (e.g., CH); F is N(R^{5c}) (e.g., NH); E is N; D is C(R^{5a})(e.g., CH). In some embodiments, the compound of Formula (I) is Compound 415, or a pharmaceutically acceptable salt, solvate, hydrate, tautomer, or stereoisomer thereof.

In some embodiments, for Formula (I), A is bicyclic heterocyclyl (e.g., pyrrolidinyl); B is monocyclic heteroaryl (e.g., pyrazolyl); R¹ is N(R^{B}R^{C})— (e.g., —NH(C₃H₅)); L is absent; M is N; P is C(R²) (e.g., CH); D is S; F is N; E is C(R^{5a})(e.g., CH). In some embodiments, the compound of Formula (I) is Compound 416, or a pharmaceutically acceptable salt, solvate, hydrate, tautomer, or stereoisomer thereof.

In some embodiments, for Formula (I), A is bicyclic heterocyclyl (e.g., piperidinyl); B is monocyclic heteroaryl (e.g., pyrazolyl); R¹ is heterocyclyl (e.g., cyclopropyl); L is absent; M and P are each independently C(R²) (e.g., CH); D is S; F is N; E is C(R^{5a})(e.g., CH). In some embodiments, the compound of Formula (I) is Compound 417, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., —N(CH₃)—); M and P are each independently C(R²) (e.g., CH); F is N(R^{5c})(e.g., NH); E is N; and D is C(R^{5a})(e.g., CH). In some embodiments, the compound of Formula (I) is Compound 418, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., —NH—); M and P are each independently C(R²) (e.g., CH); F is N(R^{5c})(e.g., NH); E is N; and D is C(R^{5a})(e.g., CH). In some embodiments, the compound of Formula (I) is Compound 419, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —NCH₃)); M is N; P is C(R²) (e.g., CH); D is S; F is N; E is C(R^{5a})(e.g., CH). In some embodiments, the compound of Formula (I) is Compound 420, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., —NCH₃)); M is N; P is C(R²) (e.g., CH); D is S; F is N; E is C(R^{5a})(e.g., CH). In some embodiments, the compound of Formula (I) is Compound 421, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., —NH—)); M is N; P is C(R²) (e.g., CH); D is S; F is N; E is C(R^{5a})(e.g., CH). In some embodiments, the compound of Formula (I) is Compound 422, or a pharmaceutically acceptable salt, solvate, hydrate, tautomer, or stereoisomer thereof.

In some embodiments, for Formula (I), A is bicyclic heterocyclyl (e.g., 1,5-dimethyl-8-azabicyclo[3.2.1]octanyl); B is monocyclic heteroaryl (e.g., pyrazolyl); L is —N(R³)— (e.g., N(CH³)); M is N; P is C(R²) (e.g., CH); R⁷ is absent; D is S; F is N; and E is C(R^{5a})(e.g., CH). In some embodiments, the compound of Formula (I) is Compound 423, Compound 424, Compound 425, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R³)— (e.g., N(CH³)); M is N; P is C(R²) (e.g., CH); R⁷ is absent; D is S; F is N; and E is C(R^{5a})(e.g., CH). In some embodiments, the compound of Formula (I) is Compound 443, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., pyrazolyl); L is —N(R³)— (e.g., N(CH³)); M is N; P is C(R²) (e.g., CH); R⁷ is absent; D is S; F is N; and E is C(R^{5a})(e.g., CH). In some embodiments, the compound of Formula (I) is Compound 449, or a pharmaceutically acceptable salt, solvate, hydrate, tautomer, or stereoisomer thereof.

In some embodiments, for Formula (I), A is monocyclic heterocyclyl (e.g., 8-azabicyclo[3.2.1]octanyl); B is monocyclic heteroaryl (e.g., pyrazolyl); L is —N(R³)— (e.g., —N(CH₃)—); M is N; P is C(R²) (e.g., CH); F is N(R^{5c}) (e.g., NH); E is N; and D is C(R^{5a})(e.g., CH). In some embodiments, the compound of Formula (I) is Compound 479, or a pharmaceutically acceptable salt, solvate, hydrate, tautomer, or stereoisomer thereof.

As generally described herein, each of M and P independently refer to C(R²) or N. In some embodiments, each of M and P are independently C(R²) or N. In some embodiments, M and P are each independently C(R²), e.g., CH. In some embodiments, one of M and P is C(R²), and the other of M and P is N. In some embodiments, M is C(R²). In some embodiments, M is N. In some embodiments, P is C(R²). In some embodiments, P is N. In some embodiments, M is C(R²) (e.g., CH) and P is N. In some embodiments, M is N and P is C(R²) (e.g., CH).

In some embodiments,

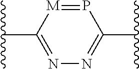

is selected from

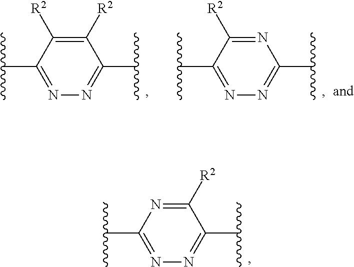

wherein R² is as defined above. In some embodiments, R² is hydrogen.

As generally described herein for Formula (II), each of X and Y independently refer to C, C(R^{5a}), C(R^{5a})(R^{5b}), N, or N(R^{5c}). In some embodiments, each of X and Y are independently C. In some embodiments, each of X and Y are C(R^{5a}). In some embodiments, X and Y are may not both be C(R^{5a}). In some embodiments, one of X and Y is C(R^{5a})(R^{5b}), and the other of X and Y is N(R^{5c}). In some embodiments, X is N(R⁵⁰) and Y is C(R^{5a})(R^{5b}). In some embodiments, X is N(R⁵⁰) and Y is C(R^{5a})(R^{5b}), where R^{5a} and R^{5b}, together with the carbon atom to which they are attached, form an oxo group. In some embodiments, one of X and Y is C(R^{5a}), and the other of X and Y is N. In some embodiments, Y is N and X is C(R^{5a}) (e.g., CH or COCH₃). In some embodiments, X is N and Y is C(R^{5a}) (e.g., CH or COCH₃). In some embodiments, one of X and Y is C(O) and the other of X and Y is NH. In some embodiments, X is NH and Y is C(O). In some embodiments, the bond between X and Y is a single bond. In some embodiments, the bond between X and Y is a double bond.

In some embodiments, when Y is N and X is C(R^{5a}) (e.g., CH), L is not —N(R³)— (e.g., —N(CH₃)—). In some embodiments, when Y is N and X is CH, L is not —N(R³)— (e.g., —N(CH₃)—). In some embodiments, when Y is N and X is C(R^{5a}) (e.g., CH), L is not —N(CH₃)—. In some embodiments, when Y is N and X is CH, L is not —N(CH₃)—. In some embodiments, when Y is N and L is —N(R³), R³ is not C₁-C₆-alkylene. In some embodiments, when Y is N and L is —N(R³), R³ is not CH₃. In some embodiments, when Y is N, X is also N. In some embodiments, when X is C(R^{5a}) (e.g., CH), Y is not N. In some embodiments, when Y comprises N, the bond between X and Y is not a double bond. In some embodiments, when Y comprises N, the bond between X and Y is a single bond. In some embodiments, when Y is N, B is not aryl or heteroaryl. In some embodiments, when Y is N, B is not aryl. In some embodiments, when Y is N, B is not heteroaryl. In some embodiments, when Y is N, B is cycloalkyl or heterocyclyl. In some embodiments, when Y is N, B is cycloalkyl. In some embodiments, when Y is N, B is heterocyclyl.

In some embodiments,

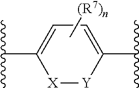

is selected from

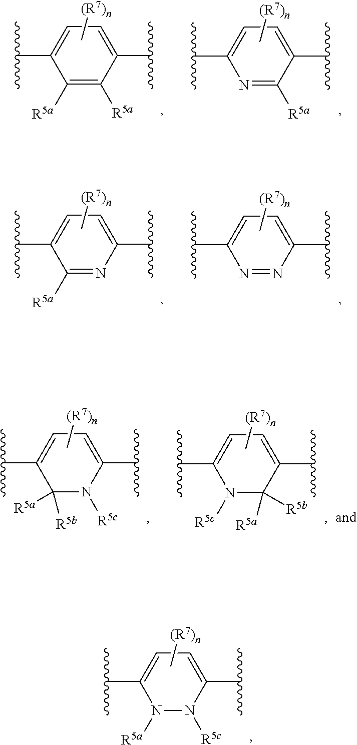

wherein each of R^{5a}, R^{5b}, R^{5c}, R⁷ and n are as defined herein.

In some embodiments,

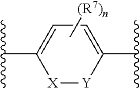

is selected from

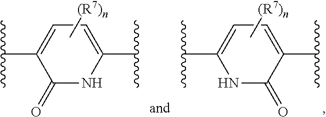

wherein each of R⁷ and n are as defined herein.

In some embodiments, R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁸. In some embodiments, R¹ is C₂-C₆-alkenyl substituted with one or more R⁸. In some embodiments, R¹ is C₂-C₆-alkynyl substituted with one or more R⁸. In some embodiments, R¹ is C₁-C₆-heteroalkyl substituted with one or more R⁸. In some embodiments, R¹ is C₁-C₆-haloalkyl substituted with one or more R⁸. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁸. In some embodiments, R¹ is heterocyclyl substituted with one or more R⁸. In some embodiments, R¹ is aryl substituted with one or more R⁸. In some embodiments, R¹ is C₁-C₆ alkylene-aryl substituted with one or more R⁸. In some embodiments, R¹ is C₁-C₆ alkenylene-aryl substituted with one or more R⁸. In some embodiments, R¹ is C₁-C₆ alkylene-heteroaryl substituted with one or more R⁸. In some embodiments, R¹ is heteroaryl substituted with one or more R⁸.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is —C(O)NR^{B}R^{C}. In some embodiments, R¹ is —C(O)R^{D}. In some embodiments, R¹ is —C(O)OR^{D}. In some embodiments, R¹ is-SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁸.

In some embodiments, R² is hydrogen. In some embodiments, R² is C₁-C₆ alkyl. In some embodiments, R² is C₂-C₆-alkenyl. In some embodiments, R² is C₂-C₆-alkynyl. In some embodiments, R² is C₁-alkyl (e.g., methyl). In some embodiments, R² is methyl. In some embodiments, R² is —OR^{A}. In some embodiments, R² is halo (e.g., fluoro, chloro, bromo, or iodo). In some embodiments, R² is fluoro. In some embodiments, R² is cyano.

In some embodiments, R³ is hydrogen. In some embodiments, R³ is C₁-C₆ alkyl. In some embodiments, R³ is C₁-C₆ haloalkyl. In some embodiments, R³ is C₁-alkyl (e.g., methyl). In some embodiments, R³ is methyl.

In some embodiments, R⁴ is C₁-C₆-alkyl. In some embodiments, R⁴ is C₁-C₆-heteroalkyl. In some embodiments, R⁴ is C₁-C₆-haloalkyl. In some embodiments, R⁴ is cycloalkyl. In some embodiments, R⁴ is halo (e.g., fluoro, chloro, bromo, or iodo). In some embodiments, R⁴ is cyano. In some embodiments, R⁴ is oxo. In some embodiments, R⁴ is —OR^{A}. In some embodiments, R⁴ is —NR^{B}R^{C}. In some embodiments, R⁴ is —C(O)R^{D} or —C(O)OR^{D}.

In some embodiments, R^{5a} and R^{5b} are each independently hydrogen or C₁-C₆-alkyl. In some embodiments, R^{5a} is hydrogen. In some embodiments, R^{5b} is hydrogen. In some embodiments, R^{5a} and R^{5b} are taken together to form an oxo group. In some embodiments, R^{5c} is hydrogen. In some embodiments, R^{5c} is C₁-C₆-alkyl. In some embodiments, R^{5c}, is C₁-C₆-haloalkyl (e.g., —CF₃ or —CHF₂). In some embodiments, R⁵, is —CF₃. In some embodiments, R^{5c}, is —CHF₂. In some embodiments, R^{5c} is —C(O)R^{D} (e.g., —C(O)CH₃). In some embodiments, R^{5c}, is —C(O)CH₃).

In some embodiments, R^{5d}, R^{5e}, and R^{5f} are each independently hydrogen, C₁-C₆-alkyl, halo, or R^{5d}, R^{5e} are taken together to form an oxo group. In some embodiments, R^{5d} is hydrogen. In some embodiments, R^{5e}, is hydrogen. In some embodiments, R^{5f} is hydrogen. In some embodiments, R^{5e} and R^{5f}, are together to form an oxo group.

In some embodiments, R⁷ is C₁-C₆-alkyl. In some embodiments, R⁷ is C₂-C₆-alkenyl. In some embodiments, R⁷ is C₂-C₆-alkynyl. In some embodiments, R⁷ is C₁-C₆-heteroalkyl. In some embodiments, R⁷ is C₁-C₆-haloalkyl. In some embodiments, R⁷ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R⁷ is C₁-C₆-alkyl substituted with one or more R⁹. In some embodiments, R⁷ is C₂-C₆-alkenyl substituted with one or more R⁹. In some embodiments, R⁷ is C₂-C₆-alkynyl substituted with one or more R⁹. In some embodiments, R⁷ is C₁-C₆-heteroalkyl substituted with one or more R⁹. In some embodiments, R⁷ is C₁-C₆-haloalkyl substituted with one or more R⁹. In some embodiments, R⁷ is halo, e.g., fluoro, chloro, bromo, or iodo. In some embodiments, R⁷ is fluoro. In some embodiments, R⁷ is cyano. In some embodiments, R⁷ is oxo. In some embodiments, R⁷ is NR^{B}C(O)R^{D}. In some embodiments, R⁷ is —C(O)NR^{B}R^{C}. In some embodiments, R⁷ is —C(O)R^{D}. In some embodiments, R⁷ is —SR^{E}.

In some embodiments, R⁸ is C₁-C₆-alkyl. In some embodiments, R⁸ is C₂-C₆-alkenyl. In some embodiments, R⁸ is C₂-C₆-alkynyl. In some embodiments, R⁸ is C₁-C₆-heteroalkyl. In some embodiments, R⁸ is C₁-C₆-haloalkyl. In some embodiments, R⁸ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁸ is C₁-C₆-alkyl substituted with one or more R¹¹. In some embodiments, R⁸ is C₂-C₆-alkenyl substituted with one or more R¹¹. In some embodiments, R⁸ is C₂-C₆-alkynyl substituted with one or more R¹¹. In some embodiments, R⁸ is C₁-C₆-haloalkyl substituted with one or more R¹¹. In some embodiments, R⁸ is C₁-C₆-heteroalkyl substituted with one or more R¹¹.

In some embodiments, R⁸ is cycloalkyl. In some embodiments, R⁸ is heterocyclyl. In some embodiments, R⁸ is aryl. In some embodiments, R⁸ is heteroaryl. In some embodiments, R⁸ is unsubstituted cycloalkyl, unsubstituted heterocyclyl, unsubstituted aryl, or unsubstituted heteroaryl. In some embodiments, R⁸ is cycloalkyl substituted with one or more R¹¹. In some embodiments, R⁸ is heterocyclyl substituted with one or more R¹¹. In some embodiments, R⁸ is aryl substituted with one or more R¹¹. In some embodiments, R⁸ is heteroaryl substituted with one or more R¹¹.

In some embodiments, R⁸ is halo (e.g., fluoro, chloro, bromo, or iodo). In some embodiments, R⁸ is cyano. In some embodiments, R⁸ is oxo. In some embodiments, R⁸ is —OR^{A}. In some embodiments, R⁸ is —NR^{B}R^{C}. In some embodiments, R⁸ is —NR^{B}C(O)R^{D}. In some embodiments, R⁸ is —NO₂. In some embodiments, R⁸ is —C(O)NR^{B}R^{C}. In some embodiments, R⁸ is —C(O)R^{D}. In some embodiments, R⁸ is —C(O)OR^{D}. In some embodiments, R⁸ is —SR^{E}. In some embodiments, R⁸ is —S(O)ₓR^{D}.

In some embodiments, R⁹ is C₁-C₆-alkyl. In some embodiments, R⁹ is C₂-C₆-alkenyl. In some embodiments, R⁹ is C₂-C₆-alkynyl. In some embodiments, R⁹ is C₁-C₆-heteroalkyl. In some embodiments, R⁹ is C₁-C₆-haloalkyl. In some embodiments, R⁹ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁹ is C₁-C₆-alkyl substituted with one or more R¹¹. In some embodiments, R⁹ is C₂-C₆-alkenyl substituted with one or more R¹¹. In some embodiments, R⁹ is C₂-C₆-alkynyl substituted with one or more R¹¹. In some embodiments, R⁹ is C₁-C₆-haloalkyl substituted with one or more R¹¹. In some embodiments, R⁹ is C₁-C₆-heteroalkyl substituted with one or more R¹¹.

In some embodiments, R⁹ is cycloalkyl. In some embodiments, R⁹ is heterocyclyl. In some embodiments, R⁹ is aryl. In some embodiments, R⁹ is heteroaryl. In some embodiments, R⁹ is unsubstituted cycloalkyl, unsubstituted heterocyclyl, unsubstituted aryl, or unsubstituted heteroaryl. In some embodiments, R⁹ is cycloalkyl substituted with one or more R¹¹. In some embodiments, R⁹ is heterocyclyl substituted with one or more R¹¹. In some embodiments, R⁹ is aryl substituted with one or more R¹¹. In some embodiments, R⁹ is heteroaryl substituted with one or more R¹¹.

In some embodiments, R⁹ is halo (e.g., fluoro, chloro, bromo, or iodo). In some embodiments, R⁹ is cyano. In some embodiments, R⁹ is oxo. In some embodiments, R⁹ is —OR^{A}. In some embodiments, R⁹ is —NR^{B}R^{C}. In some embodiments, R⁹ is —NR^{B}C(O)R^{D}. In some embodiments, R⁹ is —NO₂. In some embodiments, R⁹ is —C(O)NR^{B}R^{C}. In some embodiments, R⁹ is —C(O)R^{D}. In some embodiments, R⁹ is —C(O)OR^{D}. In some embodiments, R⁹ is —SR^{E}. In some embodiments, R⁹ is —S(O)ₓR^{D}.

In some embodiments, R¹⁰ is C₁-C₆-alkyl. In some embodiments, R¹⁰ is halo (e.g., fluoro, chloro, bromo, or iodo).

In some embodiments, R¹¹ is C₁-C₆-alkyl. In some embodiments, R¹¹ is C₁-C₆-heteroalkyl. In some embodiments, R¹¹ is C₁-C₆-haloalkyl (e.g., —CF₃). In some embodiments, R¹¹ is cycloalkyl. In some embodiments, R¹¹ is heterocyclyl. In some embodiments, R¹¹ is aryl.

In some embodiments, R¹¹ is heteroaryl. In some embodiments, R¹¹ is halo. In some embodiments, R¹¹ is cyano. In some embodiments, R¹¹ is oxo. In some embodiments, R¹¹ is —OR^{A}.

In some embodiments, R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are each independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R¹⁰ (e.g., 1, 2, or 3 R¹⁰).

In some embodiments, R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments R^{D} is hydrogen. In some embodiments, R^{D} is C₁-C₆ alkyl. In some embodiments, R^{D} is hydrogen. In some embodiments, R^{D} is C₁-C₆ alkyl (e.g., methylIn some embodiments, R^{D} is C₁-C₆ heteroalkyl. In some embodiments, R^{D} is C₁-C₆ haloalkyl. In some embodiments, R^{D} is cycloalkyl. In some embodiments, R^{D} is heterocyclyl. In some embodiments, R^{D} is aryl. In some embodiments, R^{D} is heteroaryl. In some embodiments, R^{D} is C₁-C₆ alkylene-aryl (e.g., benzyl). In some embodiments, R^{D} is C₁-C₆ alkylene-heteroaryl.

In some embodiments, m is an integer between 0 and 2 (e.g., 0, 1, or 2). In some embodiments, m is 0. In some embodiments, m is 1. In some embodiments, m is 2. In some embodiments, n is an integer between 0 and 4 (e.g., 0, 1, 2, 3, or 4). In some embodiments, n is 0. In some embodiments, n is 1. In some embodiments, n is 2. In some embodiments, n is 3. In some embodiments, n is 4. In some embodiments, x is an integer between 0 and 2 (e.g., 0, 1, or 2). In some embodiments, x is 0. In some embodiments, x is 1. In some embodiments, x is 2.

In some embodiments, the compound of Formula (II) is a compound of Formula (II-a):

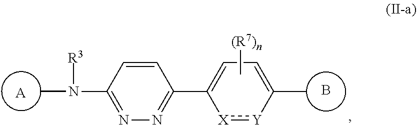

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; M and P are each independently C(R²) or N; X and Y are each independently C(R^{5a}) or N, wherein X and Y may not both be C(R^{5a}); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D} —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; R^{5a} is hydrogen, C₁-C₆-alkyl, C₂-C₆-alkenyl, C₂-C₆-alkynyl, C₁-C₆-heteroalkyl, C₁-C₆-haloalkyl, cycloalkyl, halo, cyano, oxo, —OR^{A1}, —NR^{B}R^{C}, —C(O)R^{D}, or —C(O)OR^{D}; each R^{5c} is hydrogen, C₁-C₆-alkyl, C₁-C₆-haloalkyl, or C(O)R^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹ or two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; RF is hydrogen or C₁-C₆-alkyl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is independently C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; n is 0, 1, 2, 3, or 4; and x is 0, 1, or 2.

In some embodiments, the compound of Formula (II) is a compound of Formula (II-b):

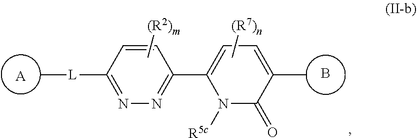

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; R^{5c} is hydrogen, C₁-C₆-alkyl, C₁-C₆-haloalkyl, or C(O)R^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or two R⁷ groups, together with the atoms to which they are attached, form a 4-7-membered cycloalkyl, heterocyclyl, aryl, or heteroaryl, wherein each cycloalkyl, heterocyclyl, aryl, and heteroaryl is optionally substituted with one or more R⁹; R¹ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is O, 1, or 2; n is 0, 1, or 2; and x is O, 1, or 2.

In some embodiments, the compound of Formula (II) is a compound of Formula (II-c):

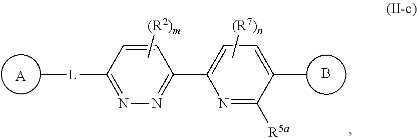

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; R^{5a} is hydrogen, C₁-C₆-alkyl, C₂-C₆-alkenyl, C₂-C₆-alkynyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R^{B}; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, or 2; n is 0, 1, 2, 3, or 4; and x is 1 or 2.

In some embodiments, the compound of Formula (II) is a compound of Formula (II-d):

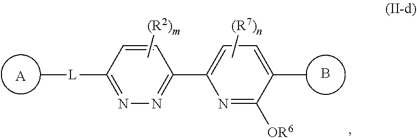

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; R⁶ is C₁-C₆-alkyl, C₂—C₆-alkenyl, C₂-C₆-alkynyl, C₁-C₆-heteroalkyl, C₁-C₆-haloalkyl, cycloalkyl, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆-alkenyl, C₂-C₆-alkynyl, C₁-C₆-heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁸; or R^{B} and R^{C} together with the nitrogen atom to which they are attached form a 3-7-membered heterocyclyl or heteroaryl, wherein each heterocyclyl or heteroaryl is optionally substituted with one or more R^{B}; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, or 2; n is 0, 1, 2, 3, or 4; and x is 1 or 2.

In some embodiments, A is heterocyclyl optionally substituted with one or more R¹. In some embodiments, A is bicyclic heterocyclyl. In some embodiments, A is monocyclic nitrogen-containing heterocyclyl. In some embodiments, A is bicyclic nitrogen-containing heterocyclyl. In some embodiments, A is optionally substituted piperidinyl. In some embodiments, A is optionally substituted azabicyclo[3.2.1]octanyl.

In some embodiments, A is selected from

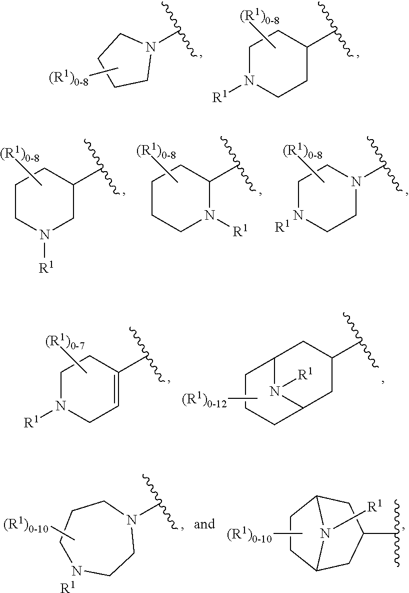

wherein R¹ is as defined herein.

In some embodiments, A is selected from

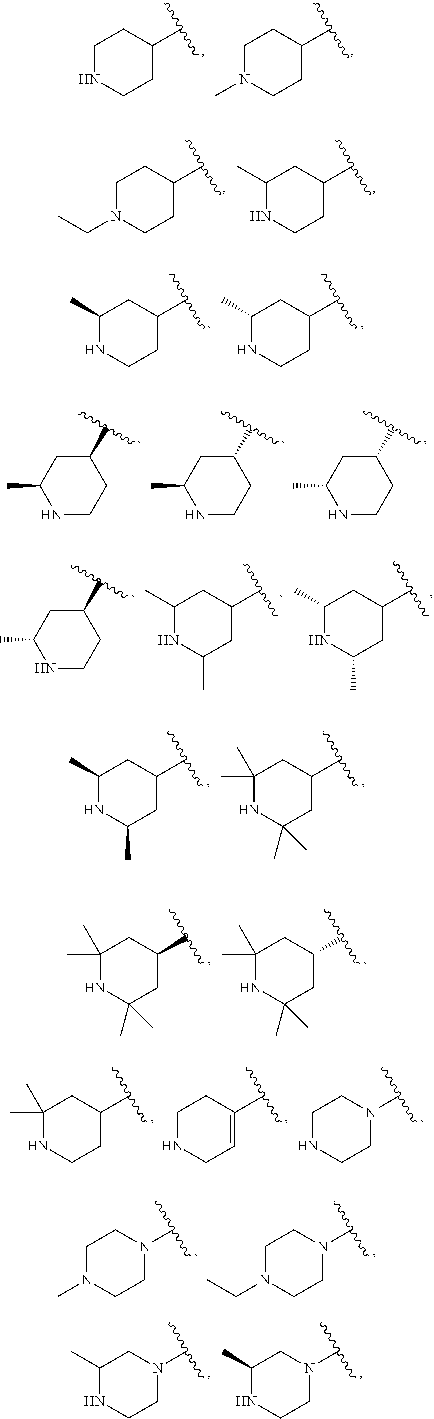

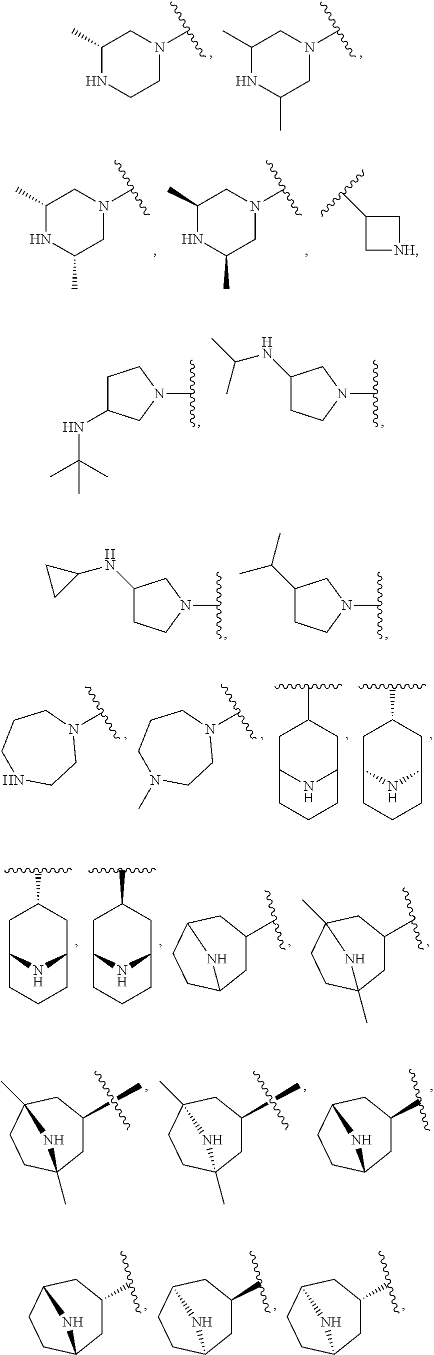

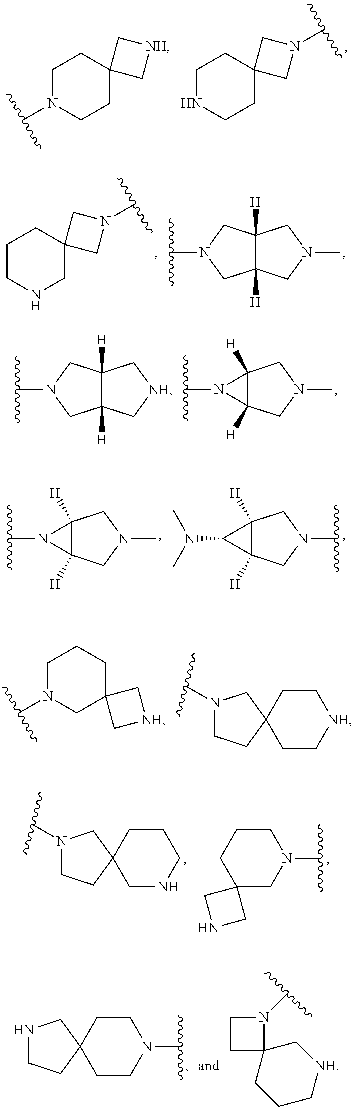

In some embodiments, A is selected from

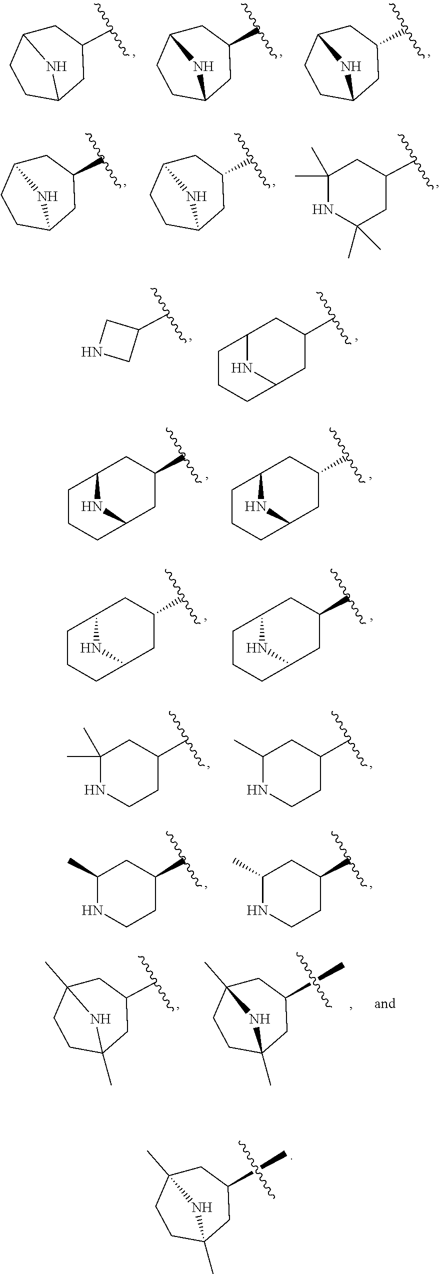

In some embodiments, A is selected from

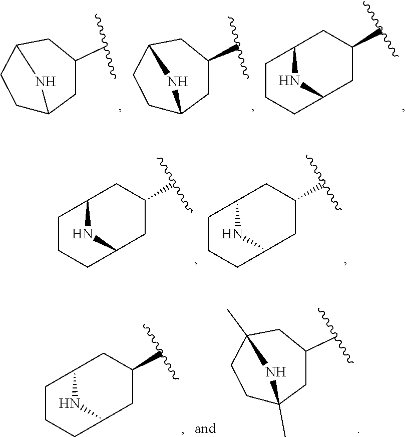

In some embodiments, A is

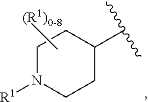

wherein each R¹ is independently hydrogen or C₁-C₆-alkyl. In some embodiments, A is

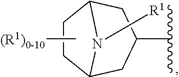

wherein each R¹ is independently hydrogen or C₁-C₆-alkyl. In some embodiments, A is

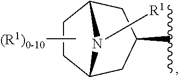

wherein each R¹ is independently hydrogen or C₁-C₆-alkyl. In some embodiments, A is

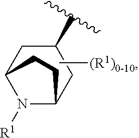

wherein each R¹ is independently hydrogen or C₁-C₆-alkyl. In some embodiments, A is

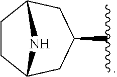 [SMILES: *[C@@H]1C[C@H]2CC[C@@H](C1)N2]

In some embodiments, A is

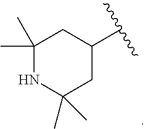 [SMILES: *C1CC(C)(C)NC(C)(C)C1]

In some embodiments, A is

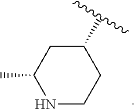 [SMILES: *[C@@H]1CCN[C@H](C)C1]

In some embodiments, A is

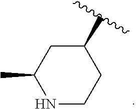 [SMILES: *[C@H]1CCN[C@@H](C)C1]

In some embodiments, A is

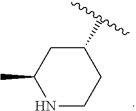 [SMILES: *[C@@H]1CCN[C@@H](C)C1]

In some embodiments, A is

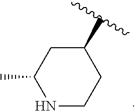 [SMILES: *[C@H]1CCN[C@H](C)C1]

In some embodiments, A is

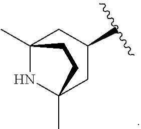

In some embodiments, A is

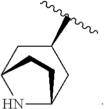

In some embodiments, A is

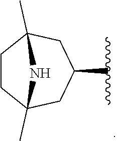 [SMILES: *[C@H]1C[C@]2(C)CC[C@](C)(C1)N2]

In some embodiments, A is

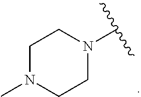 [SMILES: *N1CCN(C)CC1]

In some embodiments, A is

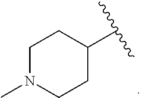 [SMILES: *C1CCN(C)CC1]

In some embodiments, L is absent. In some embodiments, L is oxygen. In some embodiments, L is nitrogen that is optionally substituted with R³. In some embodiments, L is nitrogen substituted with R³. In some embodiments, R³ is C₁-C₆ alkyl. In some embodiments, L is —N(CH₃)—. In some embodiments, L is —NH—.

In some embodiments, B is heteroaryl optionally substituted with one or more R¹. In some embodiments, B is monocyclic heteroaryl. In some embodiments, B is monocyclic nitrogen-containing heteroaryl. In some embodiments, B is optionally substituted pyrazolyl. In some embodiments, B is selected from

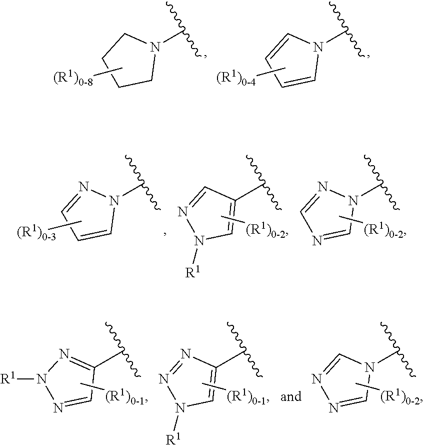

wherein R¹ is as defined herein. In some embodiments, B is selected from

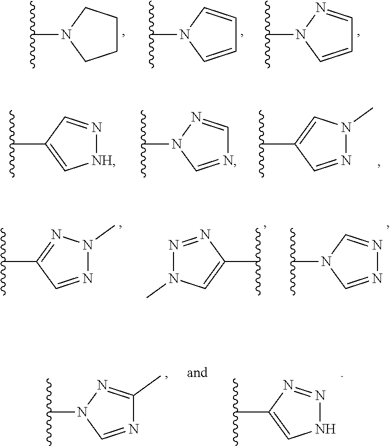

In some embodiments, B is

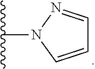 [SMILES: *n1cccn1]

In some embodiments, B is

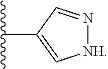 [SMILES: *c1cn[nH]c1]

In some embodiments, B is

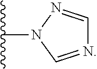 [SMILES: *n1cncn1]

In some embodiments, B is

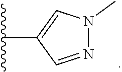 [SMILES: *c1cnn(C)c1]

In some embodiments, B is

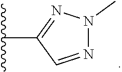 [SMILES: *c1cnn(C)n1]

In some embodiments, B is

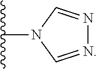 [SMILES: *n1cnnc1]

In some embodiments, B is

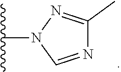 [SMILES: *n1cnc(C)n1]

In some embodiments, B is

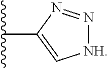 [SMILES: *c1c[nH]nn1]

In some embodiments, B is

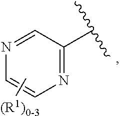

wherein R¹ is as defined herein.

In some embodiments, B is

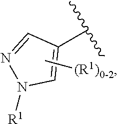

wherein each R¹ is independently hydrogen or C₁-C₆-alkyl. In some embodiments, B is

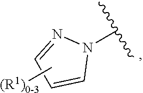

wherein each R¹ is independently hydrogen or C₁-C₆-alkyl. In some embodiments, B is

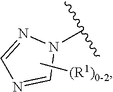

wherein each R¹ is independently hydrogen or C₁-C₆-alkyl. In some embodiments, B is

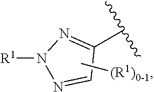

wherein each R¹ is independently hydrogen or C₁-C₆-alkyl. In some embodiments, B is

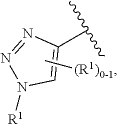

wherein each R¹ is independently hydrogen or C₁-C₆-alkyl. In some embodiments, B is

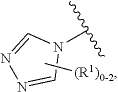

wherein each R¹ is independently hydrogen or C₁-C₆-alkyl. In some In some embodiments, B is

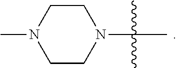 [SMILES: *N1CCN(C)CC1]

In some embodiments, B is

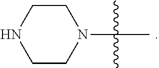 [SMILES: *N1CCNCC1]

In some embodiments, R¹ is C₁-C₆ alkyl (e.g., methyl). In some embodiments, R¹ is methyl. In some embodiments, R² is hydrogen. In some embodiments, R⁷ is hydrogen. In some embodiments, m and n are each 0. In some embodiments, R^{5c} is hydrogen.

In some embodiments, R^{5a} is hydrogen, C₁-C₆-alkyl, C₂-C₆-alkenyl, C₂-C₆-alkynyl, C₁-C₆-heteroalkyl, C₁-C₆-haloalkyl, cycloalkyl, halo, cyano, —OR^{A}, —NR^{B}R^{C}, —C(O)R^{D}, or —C(O)OR^{D}. In some embodiments, R^{5a} is hydrogen. In some embodiments, R^{5a} is C₁-C₆-alkyl. In some embodiments, R⁵, is C₁-C₆-heteroalkyl. In some embodiments, R⁵, is C₁-C₆-haloalkyl. In some embodiments, R⁵, is cycloalkyl. In some embodiments, R⁵, is halo. In some embodiments, R⁵, is cyano. In some embodiments, R^{5a} is —OR^{A}.

In some embodiments, the compound of Formula (II) is selected from a compound in Table 2, or a pharmaceutically acceptable salt thereof

TABLE 2
Exemplary compounds of Formula (II)

| Compound No. | Structure |
|---|---|
| 900 | 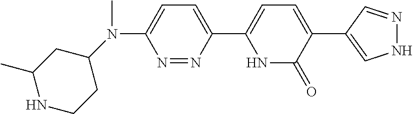 [SMILES: CC1CC(N(C)c2ccc(-c3ccc(-c4cn[nH]c4)c(=O)[nH]3)nn2)CCN1] |
| 901 | 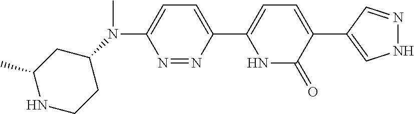 [SMILES: C[C@@H]1C[C@H](N(C)c2ccc(-c3ccc(-c4cn[nH]c4)c(=O)[nH]3)nn2)CCN1] |
| 902 | 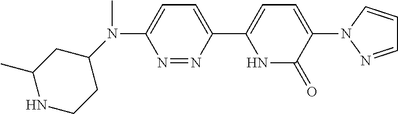 [SMILES: CC1CC(N(C)c2ccc(-c3ccc(-n4cccn4)c(=O)[nH]3)nn2)CCN1] |
| 903 | 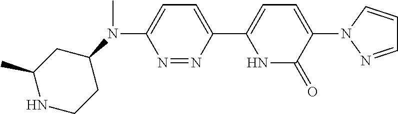 [SMILES: C[C@H]1C[C@@H](N(C)c2ccc(-c3ccc(-n4cccn4)c(=O)[nH]3)nn2)CCN1] |
| 904 | 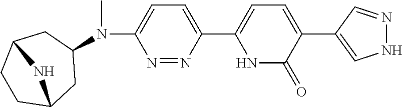 [SMILES: CN(c1ccc(-c2ccc(-c3cn[nH]c3)c(=O)[nH]2)nn1)[C@@H]1C[C@H]2CC[C@@H](C1)N2] |
| 905 | 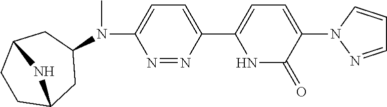 [SMILES: CN(c1ccc(-c2ccc(-n3cccn3)c(=O)[nH]2)nn1)[C@@H]1C[C@H]2CC[C@@H](C1)N2] |
| 906 | 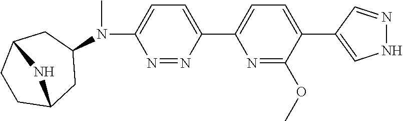 [SMILES: COc1nc(-c2ccc(N(C)[C@@H]3C[C@H]4CC[C@@H](C3)N4)nn2)ccc1-c1cn[nH]c1] |
| 907 | 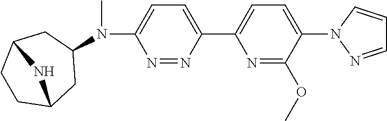 [SMILES: COc1nc(-c2ccc(N(C)[C@@H]3C[C@H]4CC[C@@H](C3)N4)nn2)ccc1-n1cccn1] |
| 908 | 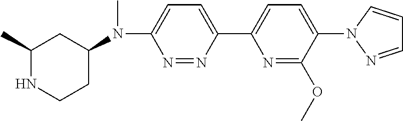 [SMILES: COc1nc(-c2ccc(N(C)[C@H]3CCN[C@@H](C)C3)nn2)ccc1-n1cccn1] |
| 909 | 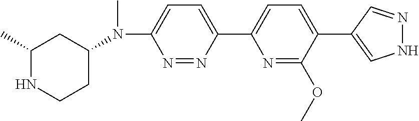 [SMILES: COc1nc(-c2ccc(N(C)[C@@H]3CCN[C@H](C)C3)nn2)ccc1-c1cn[nH]c1] |
| 910 | 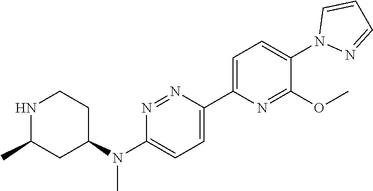 [SMILES: COc1nc(-c2ccc(N(C)[C@@H]3CCN[C@H](C)C3)nn2)ccc1-n1cccn1] |
| 911 | 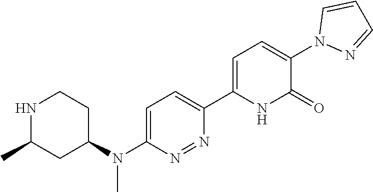 [SMILES: C[C@@H]1C[C@H](N(C)c2ccc(-c3ccc(-n4cccn4)c(=O)[nH]3)nn2)CCN1] |
| 912 | 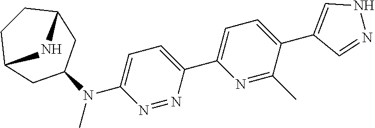 [SMILES: Cc1nc(-c2ccc(N(C)[C@@H]3C[C@H]4CC[C@@H](C3)N4)nn2)ccc1-c1cn[nH]c1] |
| 913 | 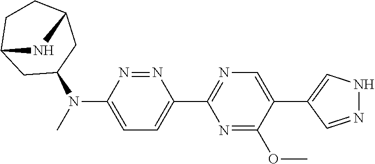 [SMILES: COc1nc(-c2ccc(N(C)[C@@H]3C[C@H]4CC[C@@H](C3)N4)nn2)ncc1-c1cn[nH]c1] |
| 914 | 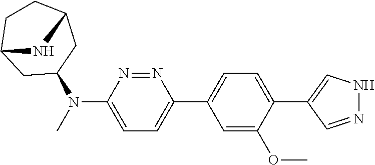 [SMILES: COc1cc(-c2ccc(N(C)[C@@H]3C[C@H]4CC[C@@H](C3)N4)nn2)ccc1-c1cn[nH]c1] |
| 915 | 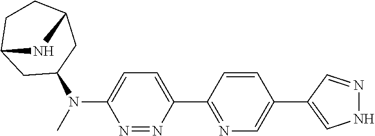 [SMILES: CN(c1ccc(-c2ccc(-c3cn[nH]c3)cn2)nn1)[C@@H]1C[C@H]2CC[C@@H](C1)N2] |
| 916 | 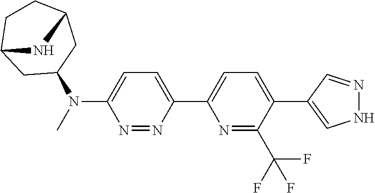 [SMILES: CN(c1ccc(-c2ccc(-c3cn[nH]c3)c(C(F)(F)F)n2)nn1)[C@@H]1C[C@H]2CC[C@@H](C1)N2] |
| 917 | 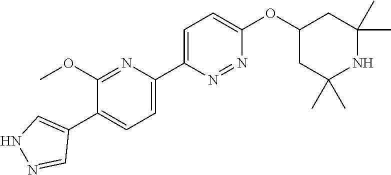 [SMILES: COc1nc(-c2ccc(OC3CC(C)(C)NC(C)(C)C3)nn2)ccc1-c1cn[nH]c1] |
| 918 | 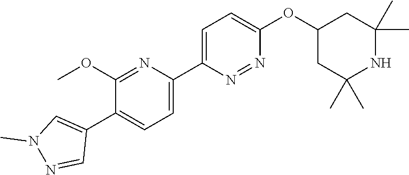 |
| 919 | 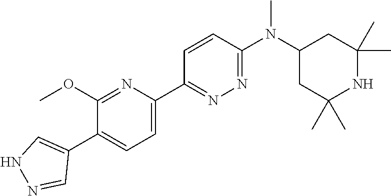 [SMILES: COc1nc(-c2ccc(N(C)C3CC(C)(C)NC(C)(C)C3)nn2)ccc1-c1cn[nH]c1] |
| 920 | 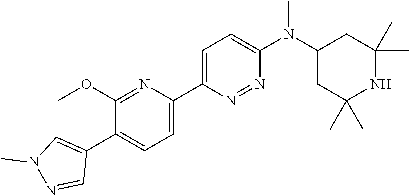 |
| 921 | 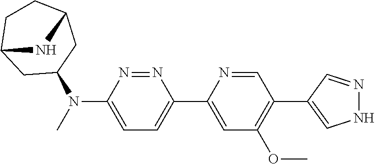 [SMILES: COc1cc(-c2ccc(N(C)[C@@H]3C[C@H]4CC[C@@H](C3)N4)nn2)ncc1-c1cn[nH]c1] |
| 922 | 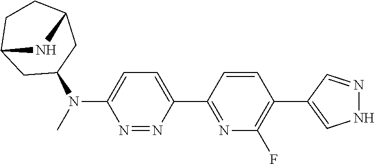 [SMILES: CN(c1ccc(-c2ccc(-c3cn[nH]c3)c(F)n2)nn1)[C@@H]1C[C@H]2CC[C@@H](C1)N2] |
| 923 | 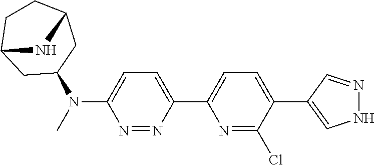 [SMILES: CN(c1ccc(-c2ccc(-c3cn[nH]c3)c(Cl)n2)nn1)[C@@H]1C[C@H]2CC[C@@H](C1)N2] |
| 924 | 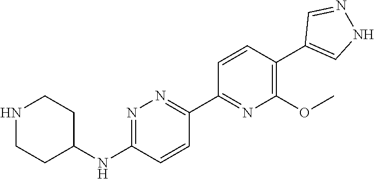 [SMILES: COc1nc(-c2ccc(NC3CCNCC3)nn2)ccc1-c1cn[nH]c1] |
| 925 | 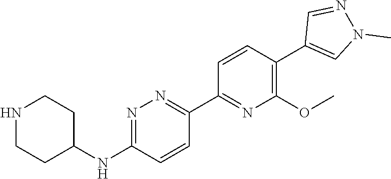 [SMILES: COc1nc(-c2ccc(NC3CCNCC3)nn2)ccc1-c1cnn(C)c1] |
| 926 | 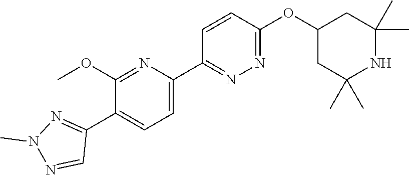 |
| 927 | 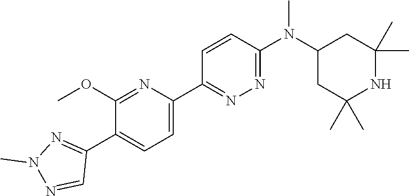 |
| 928 | 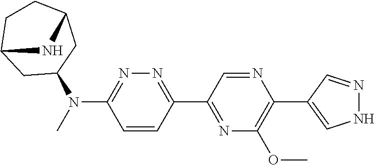 [SMILES: COc1nc(-c2ccc(N(C)[C@@H]3C[C@H]4CC[C@@H](C3)N4)nn2)cnc1-c1cn[nH]c1] |
| 929 | 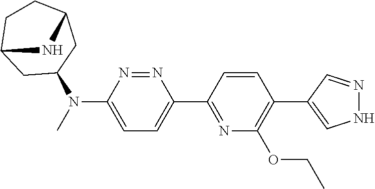 [SMILES: CCOc1nc(-c2ccc(N(C)[C@@H]3C[C@H]4CC[C@@H](C3)N4)nn2)ccc1-c1cn[nH]c1] |
| 930 | 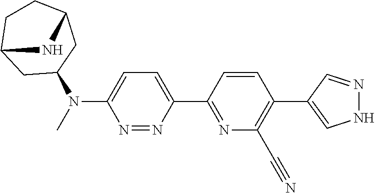 [SMILES: CN(c1ccc(-c2ccc(-c3cn[nH]c3)c(C#N)n2)nn1)[C@@H]1C[C@H]2CC[C@@H](C1)N2] |
| 931 | 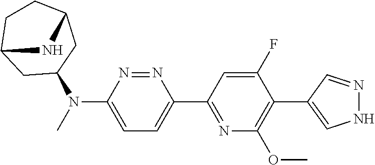 [SMILES: COc1nc(-c2ccc(N(C)[C@@H]3C[C@H]4CC[C@@H](C3)N4)nn2)cc(F)c1-c1cn[nH]c1] |
| 932 | 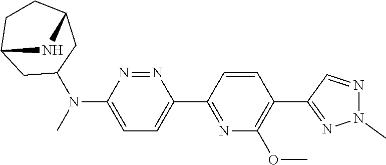 [SMILES: COc1nc(-c2ccc(N(C)C3C[C@H]4CC[C@@H](C3)N4)nn2)ccc1-c1cnn(C)n1] |
| 933 | 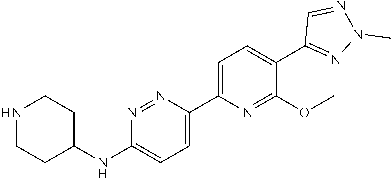 [SMILES: COc1nc(-c2ccc(NC3CCNCC3)nn2)ccc1-c1cnn(C)n1] |
| 934 | 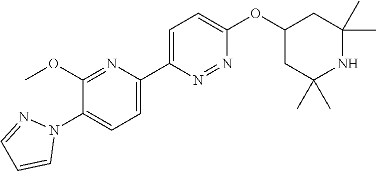 [SMILES: COc1nc(-c2ccc(OC3CC(C)(C)NC(C)(C)C3)nn2)ccc1-n1cccn1] |
| 935 | 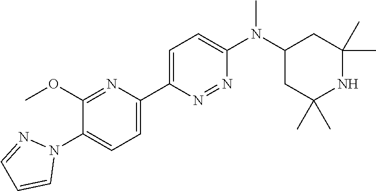 [SMILES: COc1nc(-c2ccc(N(C)C3CC(C)(C)NC(C)(C)C3)nn2)ccc1-n1cccn1] |
| 936 | 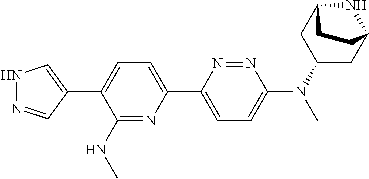 |
| 937 | 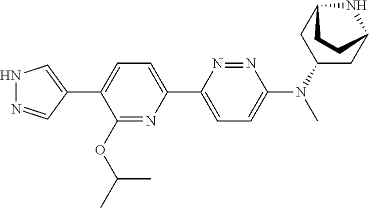 |
| 938 | 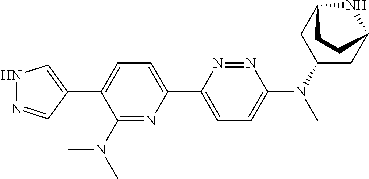 |
| 939 | 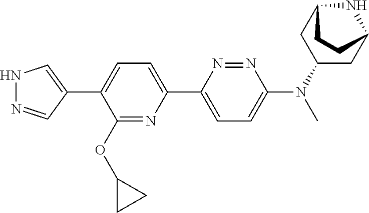 |
| 940 | 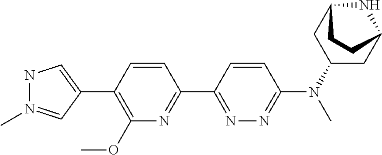 |
| 941 | 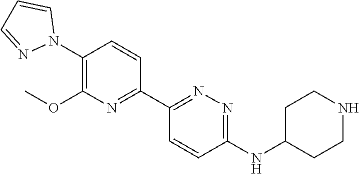 [SMILES: COc1nc(-c2ccc(NC3CCNCC3)nn2)ccc1-n1cccn1] |
| 942 | 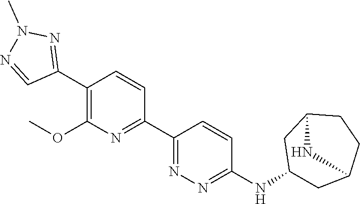 [SMILES: COc1nc(-c2ccc(N[C@@H]3C[C@H]4CC[C@@H](C3)N4)nn2)ccc1-c1cnn(C)n1] |
| 943 | 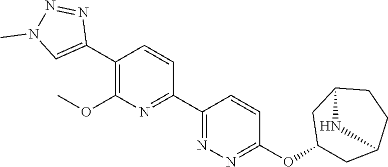 [SMILES: COc1nc(-c2ccc(O[C@@H]3C[C@H]4CC[C@@H](C3)N4)nn2)ccc1-c1cn(C)nn1] |
| 944 | 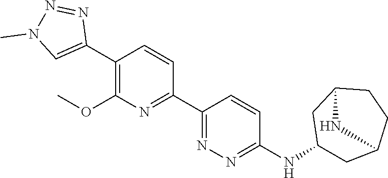 [SMILES: COc1nc(-c2ccc(N[C@@H]3C[C@H]4CC[C@@H](C3)N4)nn2)ccc1-c1cn(C)nn1] |
| 945 | 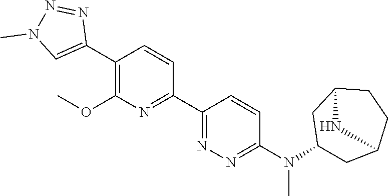 [SMILES: COc1nc(-c2ccc(N(C)[C@@H]3C[C@H]4CC[C@@H](C3)N4)nn2)ccc1-c1cn(C)nn1] |
| 946 | 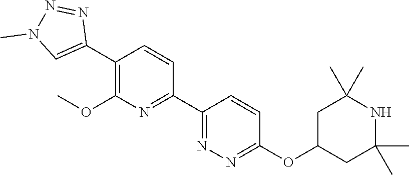 |
| 947 | 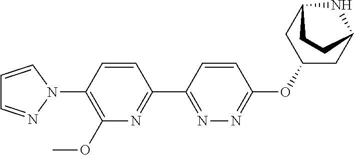 |
| 948 | 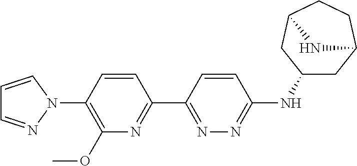 [SMILES: COc1nc(-c2ccc(N[C@@H]3C[C@H]4CC[C@@H](C3)N4)nn2)ccc1-n1cccn1] |
| 949 | 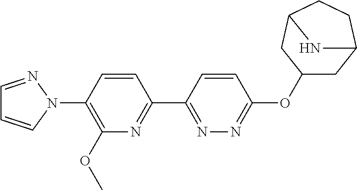 [SMILES: COc1nc(-c2ccc(OC3CC4CCC(C3)N4)nn2)ccc1-n1cccn1] |
| 950 | 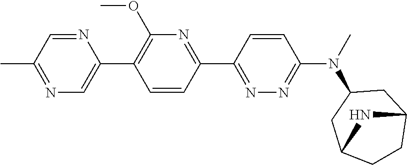 [SMILES: COc1nc(-c2ccc(N(C)[C@@H]3C[C@H]4CC[C@@H](C3)N4)nn2)ccc1-c1cnc(C)cn1] |
| 951 | 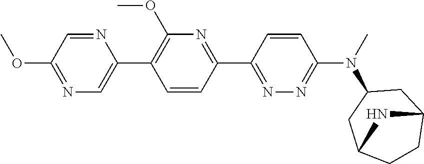 [SMILES: COc1cnc(-c2ccc(-c3ccc(N(C)[C@@H]4C[C@H]5CC[C@@H](C4)N5)nn3)nc2OC)cn1] |
| 952 | 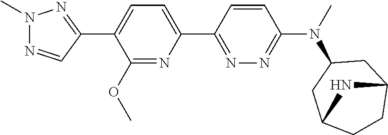 [SMILES: COc1nc(-c2ccc(N(C)[C@@H]3C[C@H]4CC[C@@H](C3)N4)nn2)ccc1-c1cnn(C)n1] |
| 953 | 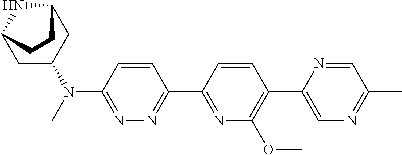 |
| 954 | 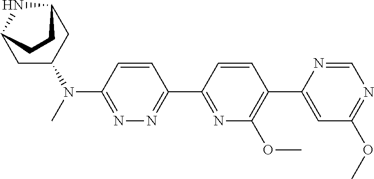 |
| 955 | 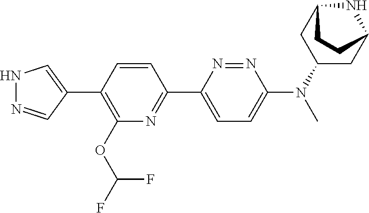 |
| 956 | 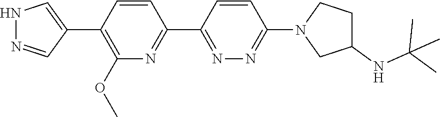 [SMILES: COc1nc(-c2ccc(N3CCC(NC(C)(C)C)C3)nn2)ccc1-c1cn[nH]c1] |
| 957 | 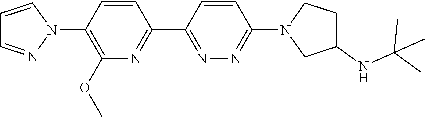 [SMILES: COc1nc(-c2ccc(N3CCC(NC(C)(C)C)C3)nn2)ccc1-n1cccn1] |
| 958 | 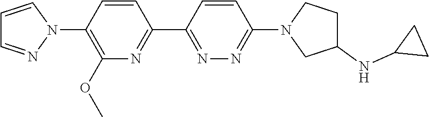 [SMILES: COc1nc(-c2ccc(N3CCC(NC4CC4)C3)nn2)ccc1-n1cccn1] |
| 959 | 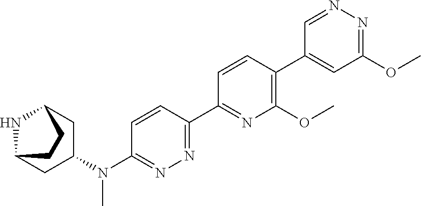 |
| 960 | 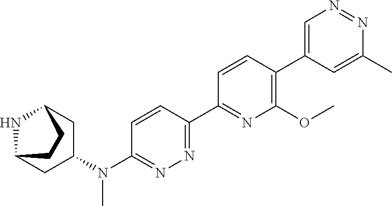 |
| 961 | 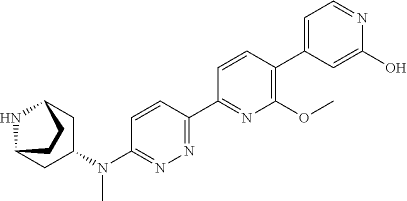 |
| 962 | 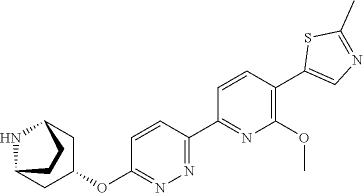 |
| 963 | 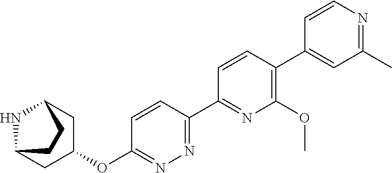 |
| 964 | 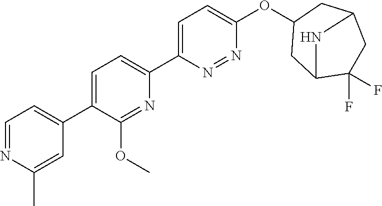 [SMILES: COc1nc(-c2ccc(OC3CC4CC(F)(F)C(C3)N4)nn2)ccc1-c1ccnc(C)c1] |
| 965 | 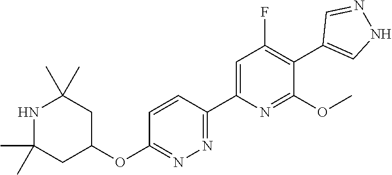 [SMILES: COc1nc(-c2ccc(OC3CC(C)(C)NC(C)(C)C3)nn2)cc(F)c1-c1cn[nH]c1] |
| 966 | 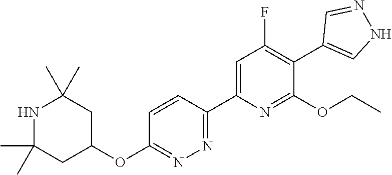 [SMILES: CCOc1nc(-c2ccc(OC3CC(C)(C)NC(C)(C)C3)nn2)cc(F)c1-c1cn[nH]c1] |
| 967 | 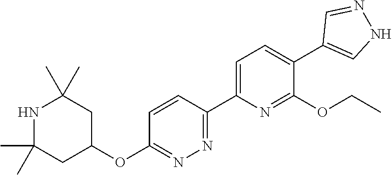 [SMILES: CCOc1nc(-c2ccc(OC3CC(C)(C)NC(C)(C)C3)nn2)ccc1-c1cn[nH]c1] |
| 968 | 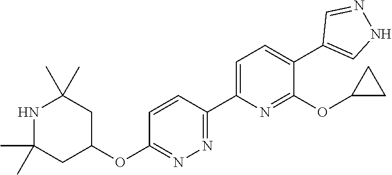 [SMILES: CC1(C)CC(Oc2ccc(-c3ccc(-c4cn[nH]c4)c(OC4CC4)n3)nn2)CC(C)(C)N1] |
| 969 | 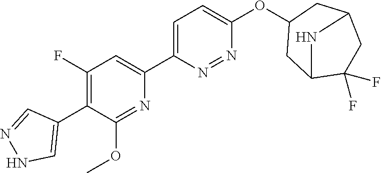 [SMILES: COc1nc(-c2ccc(OC3CC4CC(F)(F)C(C3)N4)nn2)cc(F)c1-c1cn[nH]c1] |
| 970 | 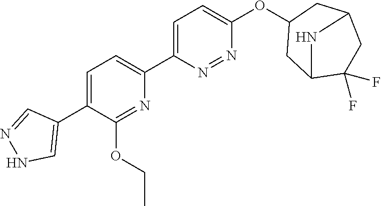 [SMILES: CCOc1nc(-c2ccc(OC3CC4CC(F)(F)C(C3)N4)nn2)ccc1-c1cn[nH]c1] |
| 971 | 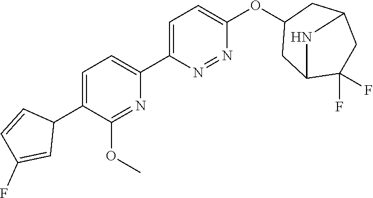 [SMILES: COc1nc(-c2ccc(OC3CC4CC(F)(F)C(C3)N4)nn2)ccc1C1C=CC(F)=C1] |
| 972 | 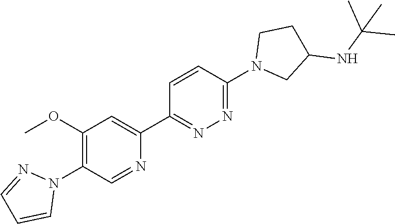 [SMILES: COc1cc(-c2ccc(N3CCC(NC(C)(C)C)C3)nn2)ncc1-n1cccn1] |
| 973 | 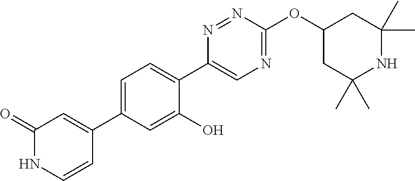 [SMILES: CC1(C)CC(Oc2ncc(-c3ccc(-c4cc[nH]c(=O)c4)cc3O)nn2)CC(C)(C)N1] |
| 974 | 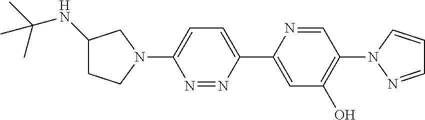 [SMILES: CC(C)(C)NC1CCN(c2ccc(-c3cc(O)c(-n4cccn4)cn3)nn2)C1] |
| 975 | 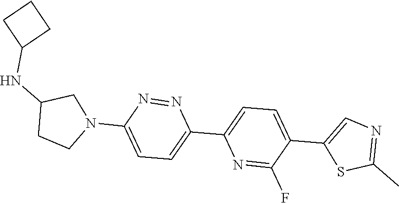 [SMILES: Cc1ncc(-c2ccc(-c3ccc(N4CCC(NC5CCC5)C4)nn3)nc2F)s1] |
| 976 | 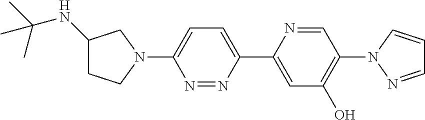 [SMILES: CC(C)(C)NC1CCN(c2ccc(-c3cc(O)c(-n4cccn4)cn3)nn2)C1] |
| 977 | 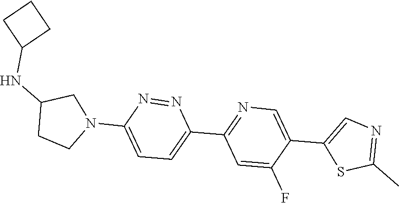 [SMILES: Cc1ncc(-c2cnc(-c3ccc(N4CCC(NC5CCC5)C4)nn3)cc2F)s1] |

In some embodiments, the compound of Formula (III) is a compound of Formula (III-a):

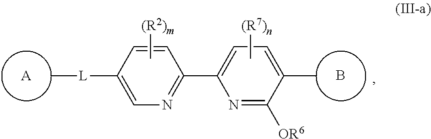

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D},—NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; R⁶ is C₁-C₆-alkyl, C₂-C₆-alkenyl, C₂-C₆-alkynyl, C₁-C₆-heteroalkyl, C₁-C₆-haloalkyl, cycloalkyl, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, or 2; n is 0, 1, or 2; and x is 1 or 2.

In some embodiments, the compound of Formula (III) is a compound of Formula (III-b):

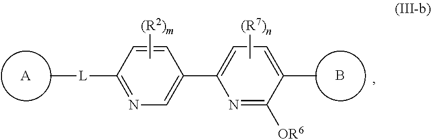

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; R⁶ is C₁-C₆-alkyl, C₂-C₆-alkenyl, C₂-C₆-alkynyl, C₁-C₆-heteroalkyl, C₁-C₆-haloalkyl, cycloalkyl, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, or 2; n is 0, 1, or 2; and x is 1 or 2.

In some embodiments, the compound of Formula (III) is a compound of Formula (III-c):

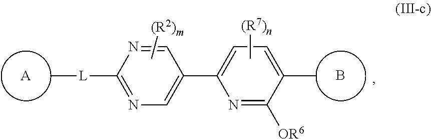

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; R⁶ is C₁-C₆-alkyl, C₂-C₆-alkenyl, C₂-C₆-alkynyl, C₁-C₆-heteroalkyl, C₁-C₆-haloalkyl, cycloalkyl, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, or 2; n is 0, 1, or 2; and x is 1 or 2.

In some embodiments, the compound of Formula (III) is a compound of Formula (III-d):

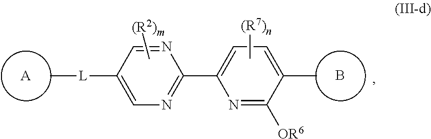

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; R⁶ is C₁-C₆-alkyl, C₂-C₆-alkenyl, C₂-C₆-alkynyl, C₁-C₆-heteroalkyl, C₁-C₆-haloalkyl, cycloalkyl, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, or 2; n is 0, 1, or 2; and x is 1 or 2.

In some embodiments, the compound of Formula (III) is a compound of Formula (III-e):

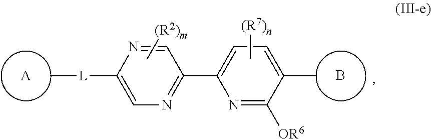

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; R⁶ is C₁-C₆-alkyl, C₂-C₆-alkenyl, C₂-C₆-alkynyl, C₁-C₆-heteroalkyl, C₁-C₆-haloalkyl, cycloalkyl, —C(O)R^{D}, or —C(O)OR^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; m is 0, 1, or 2; n is 0, 1, or 2; and x is 1 or 2.

In some embodiments, the compound of Formula (III) is a compound of Formula (III-f):

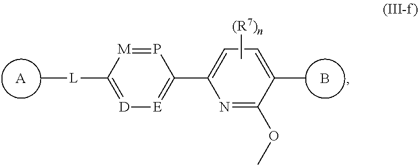

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, —O—, —C(O)—, —N(R³)—; D, E, M, and P are each independently C(R²) or N, wherein at least one of D, E, M, and P is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; n is 0, 1, or 2; and x is 1 or 2.

In some embodiments, the compound of Formula (III) is a compound of Formula (III-g):

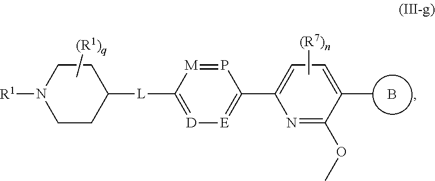

or a pharmaceutically acceptable salt, solvate, hydrate, tautomer, or stereoisomer thereof, wherein B is cycloalkyl, heterocyclyl, aryl, or heteroaryl, each of which is optionally substituted with one or more R¹; L is absent, —O—, —C(O)—, —N(R³)—; D, E, M, and P are each independently C(R²) or N, wherein at least one of D, E, M, and P is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; n is 0, 1, or 2; q is an integer between 0 and 9; and x is 1 or 2.

In some embodiments, the compound of Formula (III) (e.g., a compound of Formula (II-a), (III-b), (III-c), (III-d), (III-e), (III-f), or (III-g)), or a pharmaceutically acceptable salt thereof, is a compound depicted in Table 3.

TABLE 3
Exemplary compounds of Formula (III)

| Compound No. | Structure |
|---|---|
| 978 | 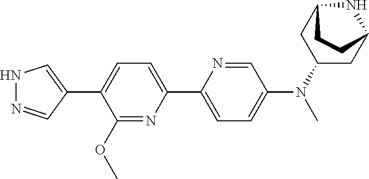 |
| 979 | 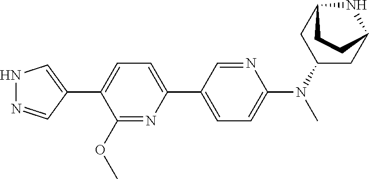 |
| 980 | 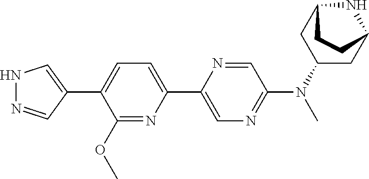 |
| 981 | 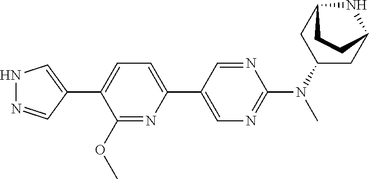 |
| 982 | 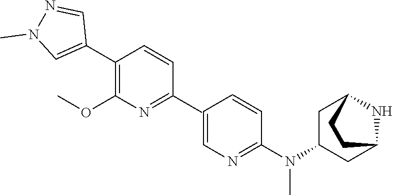 |
| 983 | 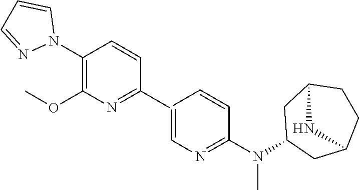 |
| 984 | 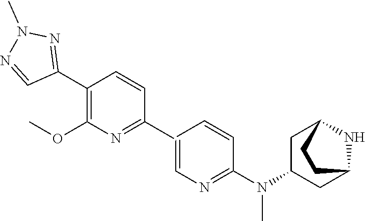 |
| 985 | 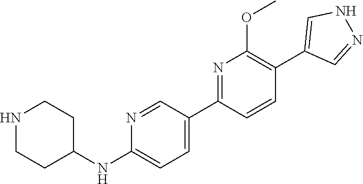 [SMILES: COc1nc(-c2ccc(NC3CCNCC3)nc2)ccc1-c1cn[nH]c1] |
| 986 | 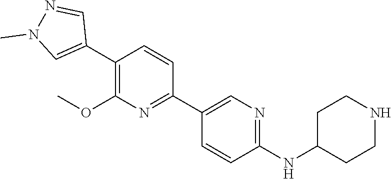 [SMILES: COc1nc(-c2ccc(NC3CCNCC3)nc2)ccc1-c1cnn(C)c1] |
| 987 | 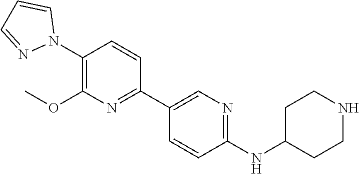 [SMILES: COc1nc(-c2ccc(NC3CCNCC3)nc2)ccc1-n1cccn1] |
| 988 | 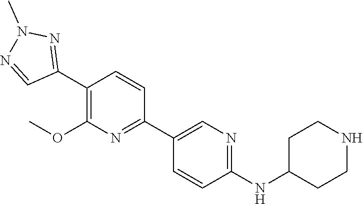 [SMILES: COc1nc(-c2ccc(NC3CCNCC3)nc2)ccc1-c1cnn(C)n1] |
| 989 | 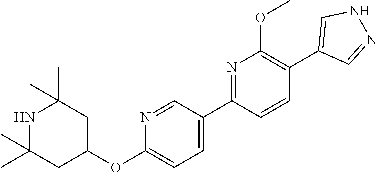 [SMILES: COc1nc(-c2ccc(OC3CC(C)(C)NC(C)(C)C3)nc2)ccc1-c1cn[nH]c1] |
| 990 | 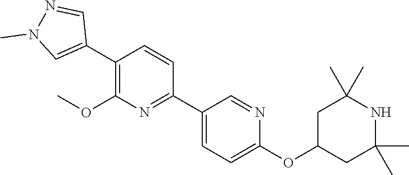 |
| 991 | 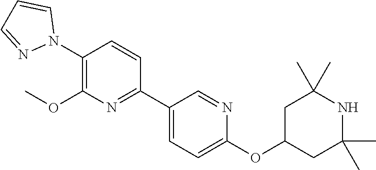 [SMILES: COc1nc(-c2ccc(OC3CC(C)(C)NC(C)(C)C3)nc2)ccc1-n1cccn1] |
| 992 | 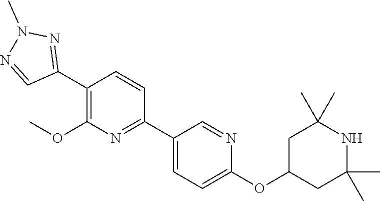 [SMILES: COc1nc(-c2ccc(OC3CC(C)(C)NC(C)(C)C3)nc2)ccc1-c1cnn(C)n1] |
| 993 | 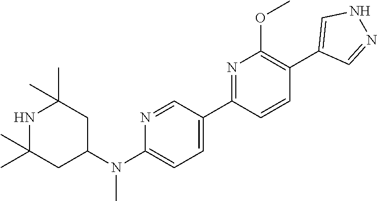 [SMILES: COc1nc(-c2ccc(N(C)C3CC(C)(C)NC(C)(C)C3)nc2)ccc1-c1cn[nH]c1] |
| 994 | 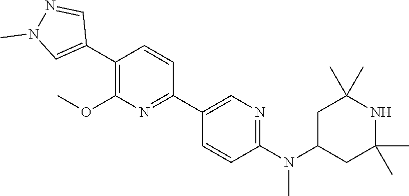 |
| 995 | 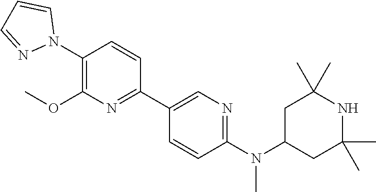 [SMILES: COc1nc(-c2ccc(N(C)C3CC(C)(C)NC(C)(C)C3)nc2)ccc1-n1cccn1] |
| 996 | 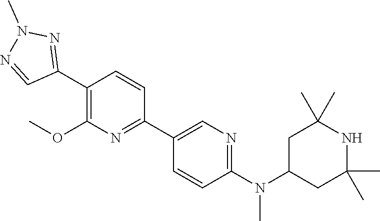 [SMILES: COc1nc(-c2ccc(N(C)C3CC(C)(C)NC(C)(C)C3)nc2)ccc1-c1cnn(C)n1] |
| 997 | 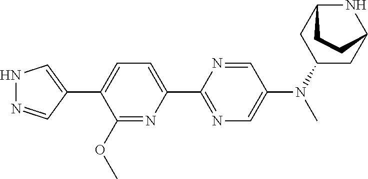 |
| 998 | 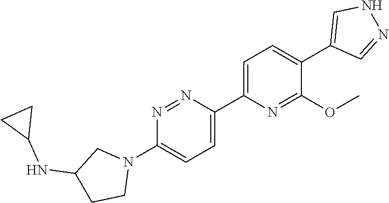 [SMILES: COc1nc(-c2ccc(N3CCC(NC4CC4)C3)nn2)ccc1-c1cn[nH]c1] |
| 999 | 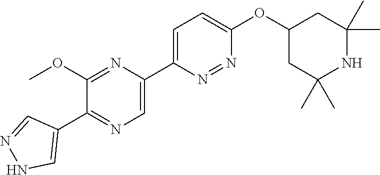 [SMILES: COc1nc(-c2ccc(OC3CC(C)(C)NC(C)(C)C3)nn2)cnc1-c1cn[nH]c1] |
| 1000 | 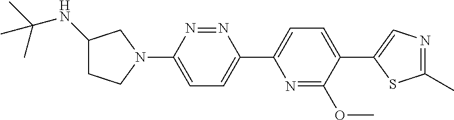 [SMILES: COc1nc(-c2ccc(N3CCC(NC(C)(C)C)C3)nn2)ccc1-c1cnc(C)s1] |
| 1001 | 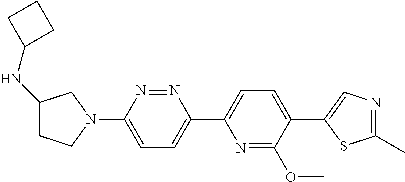 [SMILES: COc1nc(-c2ccc(N3CCC(NC4CCC4)C3)nn2)ccc1-c1cnc(C)s1] |
| 1002 | 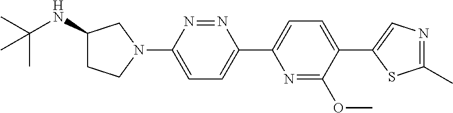 [SMILES: COc1nc(-c2ccc(N3CC[C@@H](NC(C)(C)C)C3)nn2)ccc1-c1cnc(C)s1] |
| 1003 | 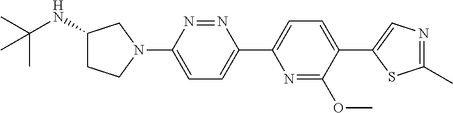 [SMILES: COc1nc(-c2ccc(N3CC[C@H](NC(C)(C)C)C3)nn2)ccc1-c1cnc(C)s1] |
| 1004 | 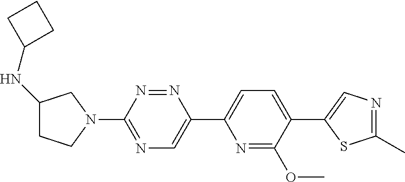 [SMILES: COc1nc(-c2cnc(N3CCC(NC4CCC4)C3)nn2)ccc1-c1cnc(C)s1] |
| 1005 | 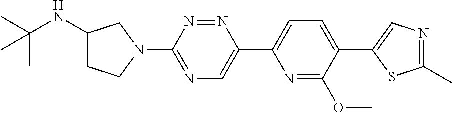 [SMILES: COc1nc(-c2cnc(N3CCC(NC(C)(C)C)C3)nn2)ccc1-c1cnc(C)s1] |
| 1006 | 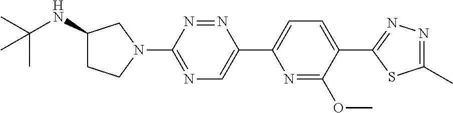 [SMILES: COc1nc(-c2cnc(N3CC[C@@H](NC(C)(C)C)C3)nn2)ccc1-c1nnc(C)s1] |
| 1007 | 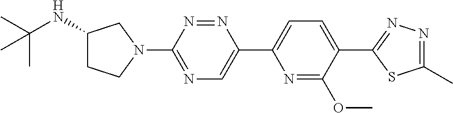 [SMILES: COc1nc(-c2cnc(N3CC[C@H](NC(C)(C)C)C3)nn2)ccc1-c1nnc(C)s1] |
| 1008 | 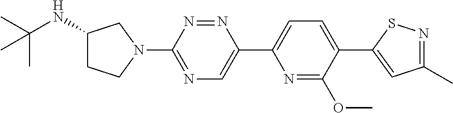 [SMILES: COc1nc(-c2cnc(N3CC[C@H](NC(C)(C)C)C3)nn2)ccc1-c1cc(C)ns1] |
| 1009 | 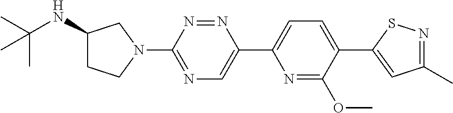 [SMILES: COc1nc(-c2cnc(N3CC[C@@H](NC(C)(C)C)C3)nn2)ccc1-c1cc(C)ns1] |
| 1010 | 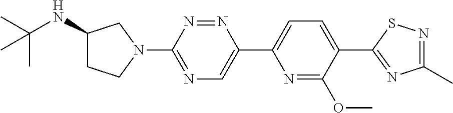 [SMILES: COc1nc(-c2cnc(N3CC[C@@H](NC(C)(C)C)C3)nn2)ccc1-c1nc(C)ns1] |
| 1011 | 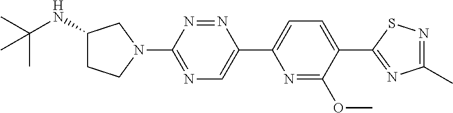 [SMILES: COc1nc(-c2cnc(N3CC[C@H](NC(C)(C)C)C3)nn2)ccc1-c1nc(C)ns1] |

### Pharmaceutical Compositions, Kits, and Administration

The present invention provides pharmaceutical compositions comprising a compound of Formula (I), (II), or (III) e.g., a compound of Formula (I), (II), or (III) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I), (II), or (III) or a pharmaceutically acceptable salt thereof, and optionally a pharmaceutically acceptable excipient. In certain embodiments, the compound of Formula (I), (II), or (III) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I), (II), or (III) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I), (II), or (III) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents.

Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I), (II), or (III) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I), (II), or (III) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I), (II), or (III). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I), (II), or (III). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I), (II), or (III), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAMJ5, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASH1L-IT1, ASNSD1, ASPM, ATAD5, ATF1, ATG4A, ATG16L2, ATM, ATN1, ATP11C, ATP6V1G3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOX1, BCSIL, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf111, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENPI, CENPT, CENTB2, CENTG2, CEPJ10, CEP170, CEP192, CETP, CFB, CFTR, CFH, CGN, CGNL1, CHAF1A, CHD9, CHIC2, CHL1, CHN1, CHM, CLEC16A, CLIC2, CLCN1, CLINT1, CLK1, CLPB, CLPM1, CMIP, CMYA5, CNGA3, CNOT1, CNOT7, CNTN6, COG3, COL11A1, COL11A2, COL12A1, COL14A1, COL15A1, COL17A1, COL19A1, COLLA1, COL1A2, COL2A, COL3A1, COL4A1, COL4A2, COL4A5, COL4A6, COL5A2, COL6A1, COL7A, COL9A, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF1, DNAH3, DNAH8, DNAI1, DNAJA4, DNAJC13, DNAJC7, DNMT1, DNTTIP2, DOCK4, DOCK5, DOCK10, DOCK11, DOT1L, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBNl, FBX015, FBX018, FBXO38, FCGBP, FECH, FEZ2, FGA, FGD6, FGFR2, FGFRIOP, FGFRIOP2, FGFR2, FGG, FGR, FIX, FKBP3, FLI1, FLJ35848, FLJ36070, FLNA, EN1, FNBP1L, FOLH1, FOSL1, FOSL2, FOXK1, FOAM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GH1, GHR, GHV, GJA1, GLA, GLT8D1, GNA11, GNAQ, GNAS, GNB5, GOLGB1, GOLTIA, GOLTIB, GPATCHI, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXCO, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF1, IKZF3, IL1R2, IL5RA, IL7RA, IMMT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, 1TGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIFS, KIF16B, KIFSA, KIFSB, KIF9, KIN, KIR2DL5B, KIR3DL2, KIR3DL3, KIT, KLF3, KLF5, KLF7, KLF10, KLF12, KLF16, KLHL20, KLK12, KLKB1, KMT2A, KMT2B, KPNAS, KRAS, KREMEN1, KRIT1, KRT5, KRTCAP2, KYNU, L1CAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCH5, MATN2, MBD3, MCF2L2, MCM6, MDGA2, MDM4, ASXL1, FUS, SPR54, MECOM, MEF2C, MEF2D, MEGF10, MEGF11, MEMO1, MET, MGA, MGAM, MGAT4A, MGAT5, MGC16169, MGC34774, MKKS, MIB1, MIER2, MITF, MKL2, MLANA, MLH1, MLL5, MLX, MME, MPDZ, MPI, MRAP2, MRPLI1, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEKS, NEK11, NF1, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NR1D1, NR1H3, NR1H4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPA1, OPN4, OPTN, OSBPL11, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3RI, PIP5KIA, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPPIR12A, PPP3CB, PPP4C, PPP4R1L, PPP4R2, PRAME, PRC1, PRDM1, PREX1, PREX2, PRIM1, PRIM2, PRKAR1A, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUS10, PVRL2, PYGM, QRSL1, RAB11FIP2, RAB23, RAF1, RALBP1, RALGDS, RB1CC1, RBL2, RBM39, RBM45, RBPJ, RBSN, REC8, RELB, RFC4, RFT1, RFTN1, RHOA, RHPN2, RIF1, RIT1, RLN3, RMND5B, RNF11, RNF32, RNFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRP1B, RSK2, RTEL1, RTF1, RUFY1, RUNX1, RUNX2, RXRA, RYR3, SAAL1, SAE1, SALL4, SAT1, SATB2, SBCAD, SCN1A, SCN2A, SCN3A, SCN4A, SCN5A, SCN8A, SCNA, SCN11A, SCO1, SCYL3, SDC1, SDK1, SDK2, SEC24A, SEC24D, SEC31A, SEL1L, SENP3, SENP6, SENP7, SERPINA1, SETD3, SETD4, SETDB1, SEZ6, SFRS12, SGCE, SGOL2, SGPL1, SH2D1A, SH3BGRL2, SH3PXD2A, SH3PXD2B, SH3RF2, SH3TC2, SHOC2, SIPA1L2, SIPA1L3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX, SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SPI10, SPAG9, SPATA13, SPATA4, SPATS1, SPECC1L, SPDEF, SPI1, SPINK5, SPP2, SPTA1, SRF, SRM, SRP72, SSX3, SSX5, SSX9, STAG1, STAG2, STAMBPLI, STARD6, STAT1, STAT3, STAT5A, STAT5B, STAT6, STK17B, STX3, STXBP1, SUCLG2, SULF2, SUPT6H, SUPT16H, SV2C, SYCP2, SYT6, SYCP1, SYTL3, SYTL5, TAF2, TARDBP, TBC1D3G, TBC1D8B, TBC1D26, TBC1D29, TBCEL, TBK1, TBP, TBPL1, TBR1, TBX, TCEB3, TCF3, TCF4, TCF7L2, TCFL5, TCF12, TCP11L2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM156, TMEM194A, TMF1, TMPRSS6, TNFRSF10A, TNFRSF10B, TNFRSF8, TNK2, TNKS, TNKS2, TOM1L1, TOM1L2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLL5, TTLL9, TTN, TTPAL, TTR, TUSC3, TXNDC10, UBE3A, UCK1, UGT1A1, UHRF1BP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VT11A, VT11B, VWA3B, WDFY2, WDR16, WDR17, WDR26, WDR44, WDR67, WDTC1, WRN, WRNIP1, WT1, WWC3, XBP1, XRN1, XRN2, XX-FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA11P, ZNF721, ABCA5, ABHD10, ABHD13, ABHD2, ABHD6, AC000120.3, KRIT1, AC004076.1, ZNF772, AC004076.9, ZNF772, AC004223.3, RAD51D, AC004381.6, AC006486.1, ERF, AC007390.5, AC007780.1, PRKAR1A, AC007998.2, INO80C, AC009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDD1A, AC073610.3, ARF3, AC074091.1, GPN1, AC079447.1, LIPT1, AC092587.1, AC079594.2, TRIM59, AC091060.1, C18orf21, AC092143.3, MC1R, AC093227.2, ZNF607, AC093512.2, ALDOA, AC098588.1, ANAPC10, AC107871.1, CALML4, ACI14490.2, ZMYM6, AC138649.1, NIPA1, AC138894.1, CLN3, AC139768.1, AC242426.2, CHD1L, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKR1A1, AKT1, AKT1S1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDH1B1, ALDH3A1, ALDH3B2, ALDOA, ALKBH2, ALPL, AMD1, AMICA1, AMN1, AMOTL2, AMYJB, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3, RP11-127I20.7, ANKS6, ANKZF1, ANPEP, ANXA11, ANXA2, ANXA8L2, AL603965.1, AOC3, AP000304.12, CRYZL1, AP000311.1, CRYZL1, AP000893.2, RAB30, AP001267.5, ATP5MG, AP002495.2, AP003175.1, OR2AT4, AP003419.1, CLCF1, AP005263.1, ANKRD12, AP006621.5, AP006621.1, AP1G1, AP3M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5F1A, ATP5G2, ATP5J, ATP5MD, ATP5PF, ATP6AP2, ATP6V0B, ATP6V1C1, ATP6V1D, ATP7B, ATXN1, ATXA1L, IST1, ATXA3, ATXA7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCSIL, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOC1S1, RP11-644F5.10, BLOC1S6, AC090527.2, BLOC1S6, RP11-96020.4, BLVRA, BMF, BOLA1, BORCS8-MEF2B, BORCS8, BRCA1, BRD1, BRDT, BRINP3, BROX, BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, C1D, C1GALT1, C1QB, C1QTNF1, C1S, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CA5B, CABYR, CALCA, CALCOCO1, CALCOCO2, CALM1, CALM3, CALML4, RP11-315D16.2, CALN1, CALU, CANT1, CANX, CAP1, CAPN12, CAPS2, CARD8, CARHSP1, CARNS1, CASC1, CASP3, CASP7, CBFA2T2, CBS, CBY1, CCBL1, CCBL2, RBMXL1, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB1IP1, CCNC, CCND3, CCNG1, CCP110, CCR9, CCT7, CCT8, CD151, CD1D, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKN1A, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHD1L, CHD8, CHFR, ZNF605, CHIA, CHID1, CHL1, CHM, CHMP1A, CHMP3, RNF103-CHMP3, CHRNA2, CIDEC, CIRBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPS5, COPS7B, COQ8A, CORO6, COTL1, COX14, RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPT1C, CREB3L2, CREM, CRP, CRYZ, CS, AC073896.1, CS, RP11-977G19.10, CSAD, CSDE1, CSF2RA, CSGALNACT, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAF11, DCAF8, PEX19, DCLRE1C, DCTD, DCTN1, DCTN4, DCUN1D2, DDR1, DDX11, DDX19B, AC012184.2, DDX19B, RP11-529K1.3, DDX25, DDX39B, ATP6V1G2-DDX39B, SNORD84, DDX42, DDX60L, DEDD, DEDD2, DEFA1, DEFA1B, DEFA1B, DEFA3, DENND1C, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKNV, DMTF1, DMTN, DNAJC14, DNAJC19, DNAL1, DNASE1L1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RPL1-500M8.7, DZIP1L, E2F6, ECHDC1, ECSIT, ECT2, EDC3, EDEM1, EDEM2, MMP24-AS1, RP4-61404.11, EEF1AKNMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL1, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRFI1, ESR2, RP11-544I20.2, ESRRA, ESRRB, ESRRG, ETFA, ETFRF1, ETV1, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANCI, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOAM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT11, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPCI, GJB1, GJB6, GLB1L, GLI1, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLT1B, GPBP1L1, GPER1, GPR116, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCY1B3, GUK1, GULP1, GYPC, GYS1, GZF1, HAGH, HAO2, HAPLN3, HAVCR1, HAX1, HBG2, AC104389.4, HBG2, AC104389.4, HBE1, HBG2, AC104389.4, HBE1, OR51B5, HBG2, HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3, HOXB4, HOXC4, HOXD3, HOXD3, HOXD4, HPCAL1, HPS4, HPS5, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, IL11, IL18BP, IL18RAP, IL1RAP, IL1RL1, IL18R1, IL1RN, IL32, IL4I1, NUP62, AC011452.1, IL4I1, NUP62, CTC-326K19.6, IL6ST, ILVBL, IMMP1L, IMPDH1, INCA1, ING1, INIP, INPP1, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, IST1, ISYNA1, ITFG2, ITGB1BP1, ITGB7, ITIH4, RP5-966M1.6, ITPRIPL1, JADE1, JAK2, JARID2, JDP2, KANK1, KANK1, RP11-31F19.1, KANK2, KANSL1L, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDM1B, KDM4A, AL451062.3, KHNYN, KIAA0040, KIAA0125, KIAA0196, KIAA0226L, PPP1R2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLCJ, APOPT1, AL139300.1, KLC4, KLHDC4, KLHDC8A, KLHL13, KLHL18, KLHL2, KLHL24, KLHL7, KLK11, KLK2, KLK5, KLK6, KLK7, KNOP1, KRBA2, AC135178.2, KRBA2, RP11-849F2.7, KRIT1, KRT15, KRT8, KTN1, KXD1, KYAT3, RBMX1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RP11-161M6.2, LMO1, LMO3, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MAD1L1, MAD1L1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3, SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGF10, MEI1, MEIS2, MELK, MET, METTL13, METTL23, MFF, MFN2, MFSD2A, MGST3, MIB2, MICAL1, MICAL3, MICOS10, NBL1, MICOS10-NBL1, MID1, MINA, MINOSI-NBL1, MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MPI, MPP1, MPP2, MPPE1, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRVI1, MS4A1, MS4A15, MS4A3, MS4A6E, MS4A7, MS4A14, MSANTD3, MSANTD4, MSH5, MSH5-SAPCD1, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRF1, MTRR, MTUS2, MUTYH, MVK, MX1, MX2, MYH10, MYL12A, MYB, MYD88, MYL5, MYLIP, MYNN, MYO15A, MYO1B, MYOM2, MZF1, N4BP2L2, NAA60, NAB1, NAE1, NAGK, NAP1L1, NAP1L4, NAPG, NARFL, NARG2, NAT1, NAT10, NBPF11, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOL10, NOL8, NONO, NPAS1, NPIPA8, RP11-1212A22.1, NPIPB3, NPIPB4, NPIPB9, NPL, NPM1, NPPA, NQO2, NR1H3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RP11-197N18.2, OLA1, OPRL1, OPTN, OR2H1, ORAI2, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARPH11, PARVG, PASK, PAX6, PBRM1, PBXIP1, PCBP3, PCBP4, AC115284.1, PCBP4, RP11-155D18.14, RP11-155D18.12, PCGF3, PCGF5, PCNP, PCSK9, PDCD10, PDCD6, AHRR, PDDC1, PDGFRB, PDIA6, PDIK1L, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4, FUT4, PKD2, PKIA, PKIG, PKM, PKN2, PLA1A, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKT1S1, PNMT, PNPLA4, PNPLA8, PNPO, PNRC1, POC1B, POFUT1, POLB, POLD1, POLH, POLI, POLL, POLR1B, POM121, POM121C, AC006014.7, POM121C, AC211429.1, POMC, POMT1, POP1, PORCN, POU5F1, PSORS1C3, PPARD, PPARG, PPHLN1, PPIL3, PPIL4, PPM1A, PPM1B, AC013717.1, PPP1CB, PPP1R11, PPP1R13L, PPP1R26, PPP1R9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF1, PSORS1C1, PSPH, PSRC1, PTBP3, PTHLH, PTK2, PTPDC1, PTPRM, PUF60, PUM2, PUS1, PUS10, PXN, PXYLP1, PYCR1, QRICH1, R3HCC1L, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAP1, RAD17, RAD51L3-RFFL, RAD51D, RAD52, RAE1, RAI14, RAI2, RALBP1, RAN, RANGAP1, RAP1A, RAP1B, RAP1GAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RP11-212D19.4, RBMS2, RBMY1E, RBPJ, RBPMS, RBSN, RCBTB2, RCC1, RCC1, SNHG3, RCCD1, RECQL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-C17orf49, RNF111, RNF123, RNF13, RNF14, RNF185, RNF216, RNF24, RNF32, RNF34, RNF38, RNF4, RNF44, RNH1, RNMT, RNPSa, RO60, ROPN1, ROPN1B, ROR2, RP1-102H19.8, C6orf163, RP1-283E3.8, CDK11A, RP11-120M18.2, PRKAR1A, RP11-133K1.2, PAK6, RP11-164J13.1, CAPN3, RP11-21J18.1, ANKRD12, RP11-322E11.6, INO80C, RP11-337C18.10, CHD1L, RP11-432B6.3, TRIM59, RP11-468E2.4, IRF9, RP11-484M3.5, UPK1B, RP11-517H2.6, CCR6, RP11-613M10.9, SLC25A51, RP11-659G9.3, RAB30, RP11-691N7.6, CTNND1, RP11-849H4.2, RP11-896J10.3, NKX2-1, RP11-96O20.4, SQRDL, RP11-986E7.7, SERPINA3, RP4-769N13.6, GPRASP1, RP4-769N13.6, GPRASP2, RP4-798P15.3, SEC16B, RP5-1021I20.4, ZNF410, RP6-109B7.3, FLJ27365, RPE, RPH3AL, RPL15, RPL17, RPL17-C18orf32, RPL17, RPL23A, RPL36, HSD11B1L, RPP38, RPS20, RPS27A, RPS3A, RPS6KA3, RPS6KC1, RPS6KL1, RPUSD1, RRAGD, RRAS2, RRBP1, RSL1D1, RSRC2, RSRP1, RUBCNL, RUNX1T1, RUVBL2, RWDD1, RWDD4, S100A13, AL162258.1, S100A13, RP1-178F15.5, S100A16, S100A4, S100A3, S100A6, S100PBP, SAA1, SACM1L, SAMD4B, SAR1A, SARAF, SARNP, RP11-762I7.5, SCAMP5, SCAP, SCAPER, SCFD1, SCGB3A2, SCIN, SCML1, SCNN1D, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISA5, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLC10A3, SLC12A9, SLC14A1, SLC16A6, SLC1A2, SLC1A6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A10, SLC6A1, SLC6A12, SLC6A9, SLC7A2, SLC7A6, SLC7A7, SLCO1A2, SLCO1C1, SLCO2B1, SLFN11, SLFN12, SLFNL1, SLMO1, SLTM, SLU7, SMAD2, SMAP2, SMARCA2, SMARCE1, AC073508.2, SMARCE1, KRT222, SMC6, SMG7, SMIM22, SMOX, SMPDL3A, SMTN, SMUJ, SMUG1, SNAP25, SNCA, SNRK, SNRPC, SNRPD1, SNRPD2, SNRPN, SNRPN, SNURF, SNUPN, SNX11, SNX16, SNX17, SOATI, SOHLH2, CCDC169-SOHLH2, CCDC169, SORBS1, SORBS2, SOX5, SP2, SPART, SPATA20, SPATA21, SPATS2, SPATS2L, SPDYE2, SPECC1, SPECC1L, SPECC1L-ADORA2A, SPECC1L-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX2IP, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, STIL, STK25, STK33, STK38L, STK40, STMN1, STON1, STON1-GTF2A1L, STRAP, STRBP, STRC, AC011330.5, STRC, CATSPER2, STRC, CATSPER2, AC011330.5, STRC, STRCP1, STT3A, STX16-NPEPL1, NPEPL1, STX5, STX6, STX8, STXBP6, STYK1, SULT1A1, SULT1A2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAF1C, TAF6, AC073842.2, TAF6, RP11-506M12.1, TAF9, TAGLN, TANK, TAPSAR1, PSMB9, TAPT1, TATDN1, TAZ, TBC1D1, TBC1D12, HELLS, TBC1D15, TBC1D3H, TBC1D3G, TBC1D5, TBC1D5, SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCP10L, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1, TF, TFP1, TGIF1, THAP6, THBS3, THOC5, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2-CTNND1, RP11-691N7.6, CTNND1, TNFAIP2, TNFAIP8L2, SCNM1, TNFRSF10C, TNFRSF19, TNFRSF8, TNFSF12-TNFSF13, TNFSF12, TNFSF13, TNFSF12-TNFSF13, TNFSF13, TNIP1, TNK2, TNNT1, TNRC18, TNS3, TOB2, TOM1L1, TOP1MT, TOP3B, TOX2, TP53, RP11-199F11.2, TP53I11, TP53INP2, TPCN1, TPM3P9, AC022137.3, TPT1, TRA2B, TRAF2, TRAF3, TRAPPC12, TRAPPC3, TREH, TREX1, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMT10B, TRMT2B, TRMT2B-AS1, TRNT1, TRO, TROVE2, TRPS1, TRPT1, TSC2, TSGA10, TSPAN14, TSPAN3, TSPAN4, TSPAN5, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE2I, UBE2J2, UBE3A, UBL7, UBXN11, UBXN7, UGDH, UGGT1, UGP2, UMAD1, AC007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWA5A, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGE1A, XAGE1B, XKR9, XPNPEP1, XRCC3, XRN2, XXYLT1, YIF1A, YIF1B, YIPF1, YIPF5, YPEL5, YWHAB, YWHAZ, YY1AP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMAT5, ZMIZ2, ZMYM6, ZMYNDJ1, ZNF10, AC026786.1, ZNF133, ZNF146, ZNF16, ZNFJ77, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXOJ6, ZNF415, ZNF418, ZNF43, ZNF433-AS1, AC008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158I13.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOAM1 gene.

Exemplary genes that may be modulated by the compounds of Formula (I), (II), or (III) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacuge (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacage (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugageg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugcgga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugagcg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagege (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugaggc (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccueg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggeg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugagge (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugageg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgcgcgcg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgegguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacace (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguaguge (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugcg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugegge (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugcgcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccuge (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucguce (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugagge (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucgc (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugcg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggece (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagagc (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550).

Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugeguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUgucegcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugeguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I), (II), or (III) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C1/C2. Human genes encoding hnRNPs include HNRNPAO, HNRNPA1, HNRNPAIL1, HNRNPAIL2, HNRNPA3, HNRNPA2B1, HNRNPAB, HNRNPBI, HNRNPC, HNRNPCLI, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I), (II), or (III) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I), (II), or (III) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I), (II), or (III) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), or (III) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I), (II), or (III). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I), (II), or (III) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I), (II), or (III) or a pharmaceutically acceptable salt, solvate, hydrate, tautomer, stereoisomer, or composition thereof In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I), (II), or (III) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I), (II), or (III), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I), (II), or (III), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I), (II), or (III) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I), (II), or (III) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I), (II), or (III) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I), (II), or (III), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I), (II), or (III) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I), (II), or (III) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I), (II), or (III) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I), (II), or (III) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I), (II), or (III) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I), (II), or (III) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I), (II), or (III) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I), (II), or (III) increases expression of the haploinsufficient gene locus. In an embodiment, a compound of Formula (I), (II), or (III) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I), (II), or (III), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I), (II), or (III) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I), (II), or (III), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I), (II), or (III) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I), (II), or (III), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I), (II), or (III) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I), (II), or (III) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), or (III) or a pharmaceutically acceptable salt thereof, e.g., as described herein, has a cell permeability measurement (Papp) of <2×10⁻⁶ cm s⁻¹. In an embodiment, a compound of Formula (I), (II), or (III) or a pharmaceutically acceptable salt thereof, e.g., as described herein, has a cell permeability measurement (Papp) of between 2-6×10⁻⁶ cm s⁻¹. In an embodiment, a compound of Formula (I), (II), or (III) or a pharmaceutically acceptable salt thereof, e.g., as described herein, has a cell permeability measurement (Papp) of Papp greater than 6×10⁻⁶ cm s⁻¹. In an embodiment, a compound of Formula (I), (II), or (III) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), or (III) or a pharmaceutically acceptable salt thereof, e.g., as described herein, has a cell efflux ratio of less than 1.5. In an embodiment, a compound of Formula (I), (II), or (III) or a pharmaceutically acceptable salt thereof, e.g., as described herein, has a cell efflux ratio of between 1.5 and 5. In an embodiment, a compound of Formula (I), (II), or (III) or a pharmaceutically acceptable salt thereof, e.g., as described herein, has a cell efflux ratio greater than 5. In an embodiment, a compound of Formula (I), (II), or (III) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, modulates the expression of a target protein (e.g., HTT or MYB) in a reference cell or sample. In an embodiment, a compound of Formula (I), (II), or (III) or a pharmaceutically acceptable salt thereof, e.g., as described herein, increases the expression of a target protein (e.g., HTT or MYB) in a reference cell or sample. In an embodiment, a compound of Formula (I), (II), or (III) or a pharmaceutically acceptable salt thereof, e.g., as described herein, decreases the expression of a target protein (e.g., HTT or MYB) in a reference cell or sample. The effect of an exemplary compound of Formula (I), (II), or (III) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I), (II), or (III) or a pharmaceutically acceptable salt thereof, e.g., as described herein, has a protein abundance response less than 100 nM. In an embodiment, a compound of Formula (I), (II), or (III) or a pharmaceutically acceptable salt thereof, e.g., as described herein, has a protein abundance response between 100-1000 nM. In an embodiment, a compound of Formula (I), (II), or (III) or a pharmaceutically acceptable salt thereof, e.g., as described herein, has a protein abundance response greater than 1000 nM. In an embodiment, a compound of Formula (I), (II), or (III) or a pharmaceutically acceptable salt thereof, e.g., as described herein, has a protein abundance response greater than 10 uM. In an embodiment, a compound of Formula (I), (II), or (III) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, modulates the viability of a target cell in a subject or sample. In an embodiment, a compound of Formula (I), (II), or (III) or a pharmaceutically acceptable salt thereof, e.g., as described herein, increases the viability of a target cell in a subject or sample. In an embodiment, a compound of Formula (I), (II), or (III) or a pharmaceutically acceptable salt thereof, e.g., as described herein, decreases the viability of a target cell in a subject or sample. In an embodiment, a compound of Formula (I), (II), or (III) or a pharmaceutically acceptable salt thereof, e.g., as described herein, does not impact the viability of a cell (e.g., is non-toxic) in a subject or sample. The effect an exemplary compound of Formula (I), (II), or (III) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I), (II), or (III) or a pharmaceutically acceptable salt thereof, e.g., as described herein, is tolerated by a target cell at a concentration of less than 100 nM. In an embodiment, a compound of Formula (I), (II), or (III) or a pharmaceutically acceptable salt thereof, e.g., as described herein, is tolerated by a target cell at a concentration of between 100-1000 nM. In an embodiment, a compound of Formula (I), (II), or (III) or a pharmaceutically acceptable salt thereof, e.g., as described herein, is tolerated by a target cell at a concentration of greater than 1000 nM. In an embodiment, a compound of Formula (I), (II), or (III) or a pharmaceutically acceptable salt thereof, e.g., as described herein, is tolerated by a target cell at a concentration of greater than 10 uM.

In some embodiments, a compound of Formula (I), (II), or (III) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,brain}) may be defined as the ratio of unbound brain-free compound concentration to unbound plasma concentration. It is calculated using the following equation:

$${K}_{p , uu , brain} = \frac{{f}_{u , brain} \times {C}_{brain}}{{f}_{u , plasma} \times {C}_{plasma}}$$

C_{brain} and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I), (II), or (III) or a pharmaceutically acceptable salt thereof, e.g., as described herein, has a Kp value of greater than 5. In an embodiment, a compound of Formula (I), (II), or (III) or a pharmaceutically acceptable salt thereof, e.g., as described herein, has a Kp value between 1 and 5. In an embodiment, a compound of Formula (I), (II), or (III) or a pharmaceutically acceptable salt thereof, e.g., as described herein, has a Kp value between 0.2-1. In an embodiment, a compound of Formula (I), (II), or (III) or a pharmaceutically acceptable salt thereof, e.g., as described herein, has a Kp value of less than 0.2. In an embodiment, a compound of Formula (I), (II), or (III) or a pharmaceutically acceptable salt thereof, e.g., as described herein, has a Kpuu value of greater than 2.5. In an embodiment, a compound of Formula (I), (II), or (III) or a pharmaceutically acceptable salt thereof, e.g., as described herein, has a Kpuu value between 0.5-2.5. In an embodiment, a compound of Formula (I), (II), or (III) or a pharmaceutically acceptable salt thereof, e.g., as described herein, has a Kpuu value between 0.1-0.5. In an embodiment, a compound of Formula (I), (II), or (III) or a pharmaceutically acceptable salt thereof, e.g., as described herein, has a Kpuu value of less than 0.1. In an embodiment, a compound of Formula (I), (II), or (III) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I), (II), or (III) or a pharmaceutically acceptable salt thereof, e.g., as described herein, exhibits selectivity for HTT, e.g., an HTT-related nucleic acid sequence. In an embodiment, a compound of Formula (I), (II), or (III) or a pharmaceutically acceptable salt thereof, e.g., as described herein, exhibits selectivity for SMN2, e.g., an SMN2-related nucleic acid sequence. In an embodiment, a compound of Formula (I), (II), or (III) or a pharmaceutically acceptable salt thereof, e.g., as described herein, exhibits selectivity for Target C, e.g., a Target C-related nucleic acid sequence. In an embodiment, a compound of Formula (I), (II), or (III) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I), (II), or (III) or a pharmaceutically acceptable salt thereof, e.g., as described herein, has a 3-fold greater selectivity for HTT over MYB. In an embodiment, a compound of Formula (I), (II), or (III) or a pharmaceutically acceptable salt thereof, e.g., as described herein, has a 3-fold greater selectivity for MYB over HTT. In an embodiment, a compound of Formula (I), (II), or (III) or a pharmaceutically acceptable salt thereof, e.g., as described herein, has a 10-fold greater selectivity for HTT over MYB. In an embodiment, a compound of Formula (I), (II), or (III) or a pharmaceutically acceptable salt thereof, e.g., as described herein, has a 10-fold greater selectivity for MYB over HTT. In an embodiment, a compound of Formula (I), (II), or (III) or a pharmaceutically acceptable salt thereof, e.g., as described herein, has a 3-fold greater selectivity for HTT over SMN2. In an embodiment, a compound of Formula (I), (II), or (III) or a pharmaceutically acceptable salt thereof, e.g., as described herein, has a 3-fold greater selectivity for SMN2 over HTT. In an embodiment, a compound of Formula (I), (II), or (III) or a pharmaceutically acceptable salt thereof, e.g., as described herein, has a 10-fold greater selectivity for HTT over SMN2. In an embodiment, a compound of Formula (I), (II), or (III) or a pharmaceutically acceptable salt thereof, e.g., as described herein, has a 10-fold greater selectivity for SMN2 over HTT. In an embodiment, a compound of Formula (I), (II), or (III) or a pharmaceutically acceptable salt thereof, e.g., as described herein, has a 3-fold greater selectivity for MYB over SMN2. In an embodiment, a compound of Formula (I), (II), or (III) or a pharmaceutically acceptable salt thereof, e.g., as described herein, has a 3-fold greater selectivity for SMN2 over MYB. In an embodiment, a compound of Formula (I), (II), or (III) or a pharmaceutically acceptable salt thereof, e.g., as described herein, has a 10-fold greater selectivity for MYB over SMN2. In an embodiment, a compound of Formula (I), (II), or (III) or a pharmaceutically acceptable salt thereof, e.g., as described herein, has a 10-fold greater selectivity for SMN2 over MYB. In an embodiment, a compound of Formula (I), (II), or (III) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I), (II), or (III), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (δ) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 m, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH-C18 (C18, Ø4.6×50 mm, 3 μm, 120 Å, 40° C.) operating in ESI(+) ionization mode; flow rate=1.2 mL/min. Mobile phase A: Water/5 mM NH₄HCO₃, Mobile phase B: CH₃CN.)

Analytical chiral HPLC: Analytical chiral HPLC was performed on a Agilent 1260 using column: CHIRALPAK IG-3, CHIRALPAK IC-3 or CHIRALPAK OJ-3, with flow rate=1.2 mL/min. Mobile phase=MTBE(DEA):EtOH=50:50).

Preparative HPLC purification: prep-HPLC purification was performed using one of the following HPLC conditions:

Condition 1: Waters-2545, Column: X-Select CSH (C18 OBD 130 Å, 5 μm, 30 mm×150 mm). Mobile Phase A: water (10 mmol/L NH₄HCO₃), Mobile Phase B: acetonitrile, Gradient:5% B to 50% B in 8 min.

Condition 2: Shimadzu, Column: XBridge Prep C18 OBD Column, 5 μm, 19 mm×150 mm; Mobile Phase A: water (10 mmol/L NH₄HCO₃), Mobile Phase B: methanol, Gradient 1: 30% B up to 50% in 10 min. Gradient 2: 25% B to 55% B in 7 min.

Condition 3: Shimadzu, Column: Xselect CSH OBD Column, 30 mm×150 mm, 5 um, Mobile Phase A: Water (10 mmol/L NH₄HCO₃), Mobile Phase B: acetonitrile, Gradient 1: 10% Phase B up to 60% in 8 min; Gradient 2: 5% Phase B up to 40% in 8 min. Gradient 3: 5% to 40% in 6 min. Gradient 4: 10% B to 50% B in 8 min.

Condition 4: Shimadzu, Column: XBridge Prep OBD C18 Column, 30×150 mm, 5 μm; Mobile Phase A: water (10 mmol/L NH₄HCO₃), Mobile Phase B: acetonitrile; Gradient 1: 10 B to 44 B in 8 min; Gradient 2: 3 B to 33 B in 6 min; Gradient 3: 5 B to 35 B in 8 min; Gradient 4: 5 B to 24 B in 8 min; Gradient 5: 5 B to 43 B in 6 min; Gradient 6: hold 5 B in 2 min, up to 55 B in 6 min; Gradient 7: 5% B to 36% B in 8 min; Gradient 8: 10% B up to 65% B in 8 min; Gradient 9: 5% B to 32% B in 8 min; Gradient 10: 5% B to 50% B in 8 min; Gradient 11: 5% B to 42% B in 10 min. Gradient 12: 5% B to 45% B in 8 min. Gradient 13: 5% B to 65% B in 8 min. Gradient 14: 10% B to 65% B in 8 min. Gradient 15: 10% B to 55% B in 8 min.

Condition 5: Column: Xselect CSH OBD Column 30*150 mm 5 um, n; Mobile Phase A: water (10 mmol/L NH₄HCO₃); Mobile Phase B: acetonitrile; Flow rate: 60 mL/min; Gradient 1: 10 B to 35 B in 10 min. Gradient 2: 5% B to 65% B.

Condition 6: Column, Xselect CSH OBD Column 30×150 mm 5 μm; Mobile Phase A: water (0.1% HCl); Mobile Phase B: acetonitrile; Gradient 1: Hold 3% phase B for 2 min, then ramp up to 23% over 6 min.

Condition 7: Column, YMC-Actus Triart C18, 30×150 mm, 5 um; Mobile Phase A: water (10 mmol/L NH₄HCO₃); Mobile Phase B: acetonitrile; Gradient 1: 5% B to 50% B in 8 min; Gradient 2: 5% B to 45% B in 8 min; Gradient 3: 5% B to 65% B in 8 min; Gradient 4: 15% B to 50% B in 8 min. Gradient 5: 10% B to 60% B in 8 min. Gradient 6: 5% B to 55% B in 8 min. Gradient 7: 10% B to 70% B in 8 min. Gradient 8: 20% B to 70% B in 8 min. Gradient 9: 5% B to 35% B in 8 min. Gradient 10: 10% B to 52% B in 8 min.

Condition 8: Column: Xselect CSH OBD Column 30×150 mm 5 um; Mobile Phase A: water (0.05% HCl), Mobile Phase B: acetonitrile; Flow rate: 60 mL/min; Gradient 1: 3% B to 3% B in 2 min; Gradient 2: 3% B to 23% B in 8 min; Gradient 3: 5% B to 40% B in 8 min; Gradient 4: 3% B to 35% B in 8 min; Gradient 5: 3% B to 43% B in 8 min. Gradient 6: 5% B to 45% B in 8 min.

Condition 9: Column: XBridge Shield RP18 OBD Column, 19×150 mm, 5 μm; Mobile Phase A: water (0.05% TFA), Mobile Phase B: acetonitrile; Flow rate: 20 mL/min; Gradient 1: 5% B to 22% B in 8 min.

Condition 10: Column: SunFire Prep C18 OBD Column, 19×150 mm, 5 μm 10 nm; Mobile Phase A: water (0.05% TFA), Mobile Phase B: acetonitrile; Flow rate: 20 mL/min; Gradient 1: 10% B to 55% B in 6.9 min.

Condition 11: Column: XBridge Shield RP18 OBD Column, 19×150 mm, 5 μm; Mobile Phase A: water (0.05% NH₃H₂O), Mobile Phase B: acetonitrile; Flow rate: 20 mL/min; Gradient 1: 36% B to 54% B in 8 min. Gradient 2: 55% B to 85% B in 8 min.

Condition 12: Column: XBridge Prep OBD C18 Column, 30×150 mm 5 μm; mobile phase: A: Water (0.05% HCl) and B: acetonitrile. Gradient 1: 5% B to 35% in 6 min. Gradient 2: 5% B to 40% B in 8 min.

Condition 13: SunFire Prep C18 OBD Column, 19×150 mm, 5 μm 10 nm; Mobile Phase A: water (0.05% HCl), Mobile Phase B: acetonitrile; Flow rate: 20 mL/min; Gradient 1: 15% B to 25% B in 7 min. Gradient 2: 10% B to 20% B in 7 min.

Condition 14: Column: Phenomenex luna C18 80×40 mm×3 μm; Mobile Phase A: Water (HCl) Mobile Phase B: CH₃CN; Gradient 1: 1% B to 40% B in 7 min; Gradient 2: 5% B to 35% B in 8 min; Gradient 3: 10% B to 30% B in 7 min; Gradient 4: 10% B to 50% B in 8 min; Gradient 5: 1% B to 30% B in 8 min; Gradient 6: 24% B to 45% B in 8 min; Gradient 7: 5% B to 45% B in 8 min; Gradient 8: 5% B to 20% B in 8 min; Gradient 9: 20% B to 40% B in 8 min; Gradient 10: 10% B to 40% B in 8 min.

Preparative chiral HPLC: purification by chiral HPLC was performed on a Gilson-GX 281 using column: CHIRALPAK 1G-3, CHIRALPAK IC-3 or CHIRALPAK OJ-3.

Condition 1: Column, XBridge Prep OBD C18 Column, 30×150 mm; Mobile Phase A, water (10 mmol/L NH₄HCO₃), Mobile Phase B: acetonitrile; Gradient 1: 5% B to 50% B in 8 min.

Condition 2: Column: YMC-Actus Triart C18, 30×150 mm, 5 μm; Mobile phase: A: water (10.0 mmol/L NH₄HCO₃) and B: acetonitrile. Gradient 1: 5% B to 50% B in 8 min.

Condition 3: Column: Xselect CSH OBD Column 30×150 mm 5 μm; Mobile Phase A: Water (0.05% HCl), Mobile Phase B: acetonitrile. Gradient 1: 5% B to 40% B in 8 min. Gradient 2: Hold 3% B for 2 min, up to 25% B in 6 min. Gradient 3: Hold 3% B for 2 min, up to 43% B in 6 min.

### General Synthetic Scheme

Compounds of the present disclosure may be prepared using a synthetic protocol illustrated in any one of Schemes A-D.

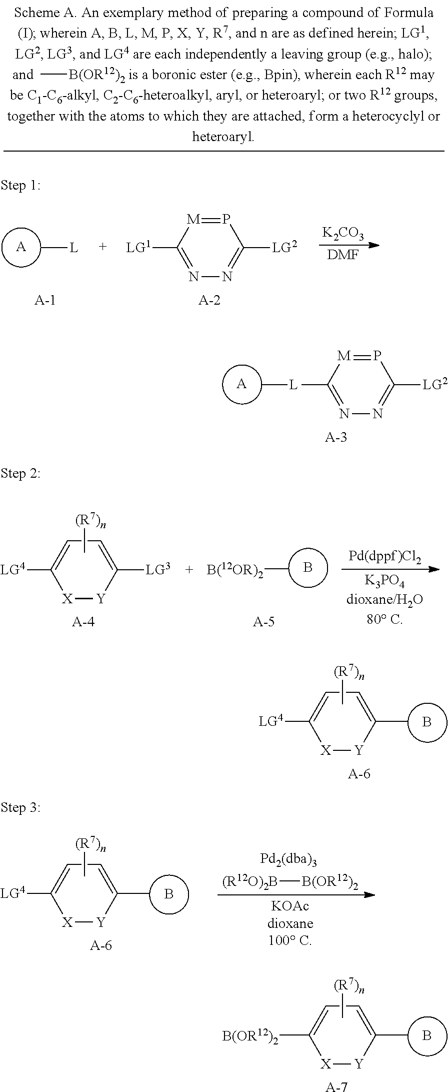

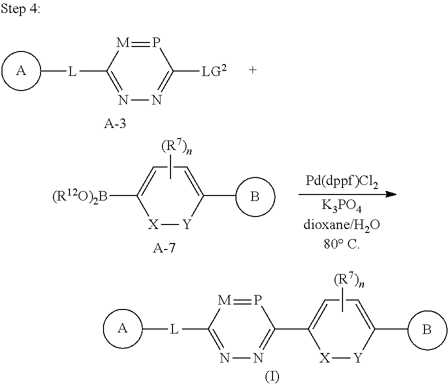

An exemplary method of preparing a compound of Formula (I) is provided in Scheme A. In this scheme, A-3 is prepared in Step 1 by incubating A-1 with A-2 in the presence of a base, for example, potassium carbonate (K₂CO₃) or sodium hydride (NaH) in N,N-dimethylformamide (DMF) or another suitable reagent. In some instances, A-3 is prepared by heating the reaction mixture to a suitable temperature, for example, 100° C. In Step 2, A-6 is prepared by incubating A-4 with A-5. Step 2 may be carried out in the presence of 1,1′-bis(diphenylphosphino)-ferrocene)palladium(II) dichloride (Pd(dppf)Cl₂), and tripotassium phosphate (K₃PO₄) or a similar reagent, for example, potassium carbonate (K₂CO₃). Alternative catalysts to Pd(dppf)Cl₂ may also be used, such as a suitable palladium catalyst (e.g., a catalyst suitable for a Suzuki reaction), for example, tetrakis(triphenylphosphine)-palladium(0) (Pd(PPh₃)₄). The coupling of A-4 and A-5 may be carried out in a mixture of dioxane and water, or a similar solvent or mixture, and heated to 80° C. or temperature sufficient to provide A-6, for example, 100° C.

In Step 3, A-7 is prepared by incubating A-6 with a reagent suitable to displace LG⁴ with a boronic ester group, such as (R¹²O)₂B-B(OR¹²)₂(e.g., bis(pinacolato)diboron (B₂pin₂)). Other common reagents for installing boronic ester groups (e.g., pinacol borane) can also be used. This reaction may involve the use of tris(dibenzylideneacetone)dipalladium(0) (Pd₂(dba)₃), and potassium acetate (KOAc) or a suitable alternative, for example, tripotassium phosphate (K₃PO₄). Step 3 may also be carried out using an alternative catalyst to Pd₂(dba)₃, such as another palladium catalyst, for example, [1,1′-bis(di-tert-butylphosphino)ferrocene]dichloropalladium(II) (Pd(dtbpf)Cl₂) or chloro(2-dicyclohexylphosphino-2′,4′,6′-triisopropyl-1,1′-biphenyl)[2-(2′-amino-1,1′-biphenyl)]palladium(II) (XPhos-Pd-G2). The reaction may be conducted in dioxane or a similar solvent, at 100° C. or a temperature sufficient to provide Fragment A-7, for example, 80° C., 90° C., 110° C., or 120° C. The reaction may be conducted in a microwave reactor.

A-3 and A-7 are coupled to provide a compound of Formula (I) in Step 4. This coupling reaction may be conducted in the presence of Pd(dppf)Cl₂, and K₃PO₄ or a similar reagent, for example tripotassium carbonate (K₃PO₄). As in Step 2, alternative catalysts to Pd(dppf)Cl₂ may be used, such as any suitable palladium catalyst, for example, tetrakis(triphenylphosphine)-palladium(0) (Pd(PPh₃)₄) or chloro(2-dicyclohexylphosphino-2′,4′,6′-triisopropyl-1,1′-biphenyl)[2-(2′-amino-1,1′-biphenyl)]palladium(II) (XPhos-Pd-G2). The reaction of Step 4 is conducted in dioxane or a mixture of dioxane and water, or other suitable solvents, and the mixture is heated to 80° C. or a temperature sufficient to provide the compound of Formula (I) or a precursor to the compound of Formula (I) with one or more protecting group(s), for example, 100° C. Compounds of Formula (I) may be purified using standard techniques and characterized using any method known in the art, such as nuclear magnetic resonance spectroscopy (NMR) or mass spectrometry (MS).

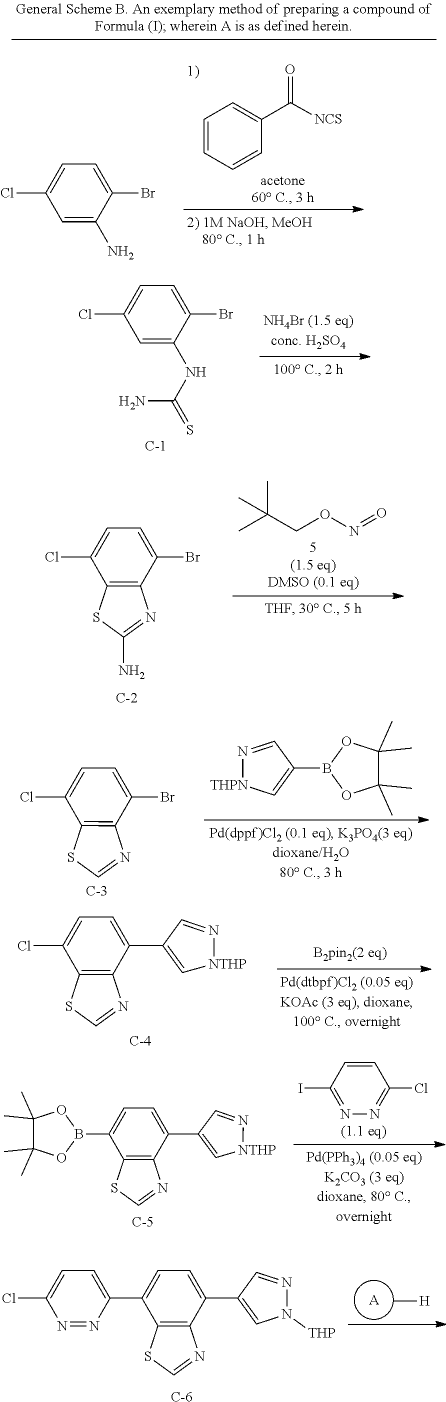

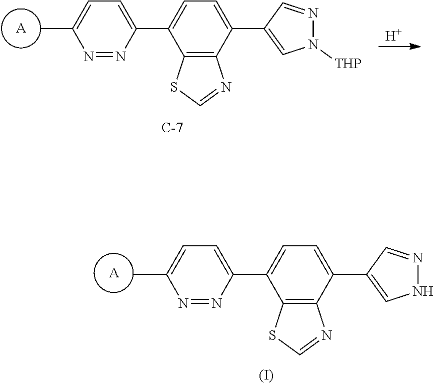

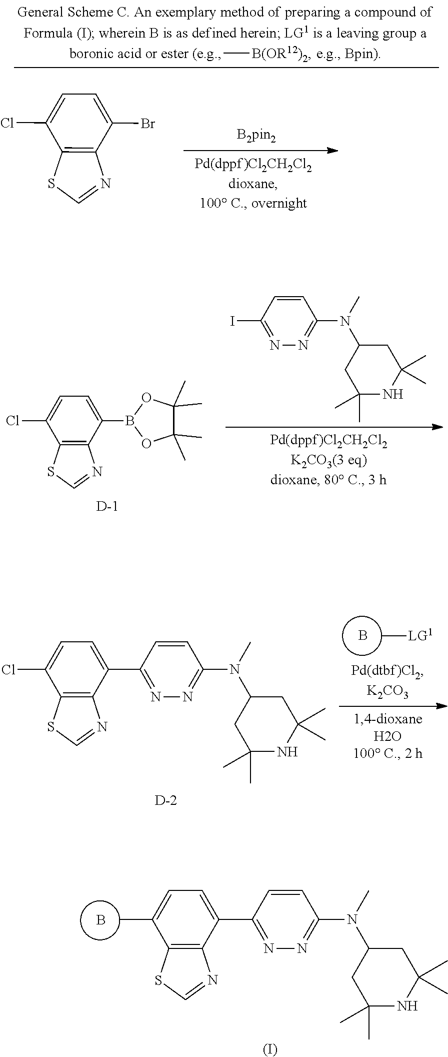

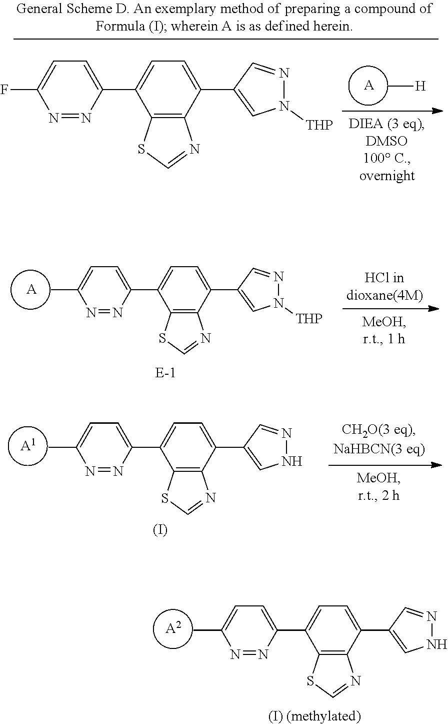

The synthesis of compounds of Formulas (I), (II), and (III) can be carried out according to protocols outlined in WO 2021/174176 or WO 2021/174170, each of which is incorporated herein by reference in its entirety.

### Example 69: Synthesis of Compound 312

### Synthesis of Intermediate C1

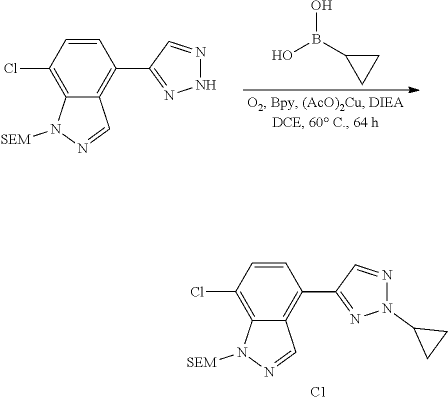

To a stirred mixture of 7-chloro-4-(1H-1,2,3-triazol-5-yl)-1-((2-(trimethylsilyl)ethoxy) methyl)-1H-indazole (700.0 mg, 2.001 mmol, 1.0 equiv) and cyclopropylboronic acid (257.8 mg, 3.002 mmol, 1.5 equiv) in dichloroethane (14 mL) was added bipyridyl (62.5 mg, 0.400 mmol, 0.2 equiv) and Cu(OAc)₂ (72.7 mg, 0.400 mmol, 0.2 equiv) at room temperature under O₂ atmosphere. The resulting mixture was stirred for 16 hr at 60° C. under O₂ atmosphere. The resulting mixture was concentrated under vacuum. The residue was purified by silica gel column chromatography, eluted with petroleum ether/ethyl acetate (1:1) to afford 7-chloro-4-(2-cyclopropyl-2H-1,2,3-triazol-4-yl)-1-((2-(trimethylsilyl)ethoxy)methyl)-1H-indazole (230 mg, C1) as a solid that was taken to the next step without further purification. LCMS (ES, m/z): 390 [M+H]⁺

### Synthesis of Intermediate C2

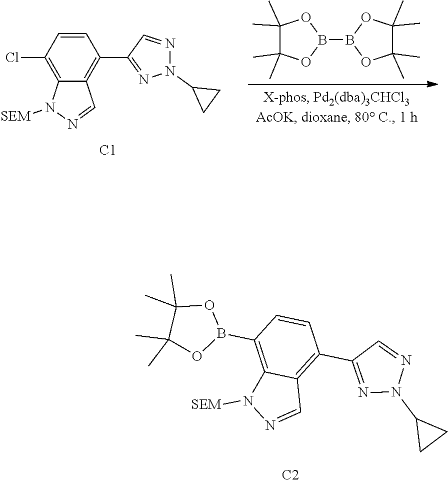

To a stirred mixture of 7-chloro-4-(2-cyclopropyl-2H-1,2,3-triazol-4-yl)-1-((2-(trimethylsilyl)ethoxy)methyl)-1H-indazole (108.0 mg, 0.277 mmol, 1.0 equiv), bis(pinacolato)diboron (105.5 mg, 0.416 mmol, 1.5 equiv) and potassium acetate (81.5 mg, 0.831 mmol, 3.0 equiv) in dioxane (2.2 mL) were added X-phos (13.2 mg, 0.028 mmol, 0.1 equiv) and Pd₂(dba)₃CHCl₃ (28.7 mg, 0.028 mmol, 0.1 equiv) at room temperature under hydrogen atmosphere. The resulting mixture was stirred for 1 hr at 80° C. under a nitrogen atmosphere. The resulting mixture was concentrated under reduced pressure. The residue was purified by silica gel column chromatography, eluted with petroleum ether/ethyl acetate (5:1) to afford 4-(2-cyclopropyl-2H-1,2,3-triazol-4-yl)-7-(4,4,5,5-tetramethyl-1,3,2-dioxaborolan-2-yl)-1-((2-(trimethylsilyl)ethoxy)methyl)-1H-indazole (125 mg, C2) as an oil that was taken to the next step without further purification. LCMS (ES, m/z): 482 [M+H]⁺

### Synthesis of Intermediate C3

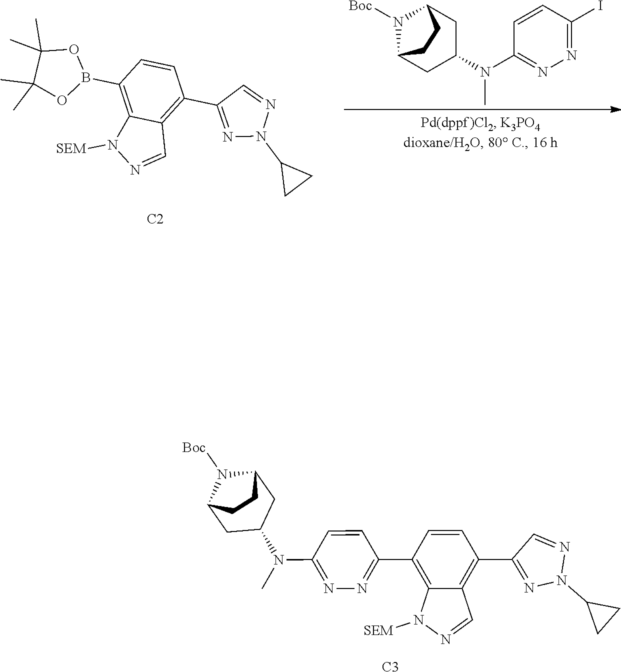

To a stirred mixture of 4-(2-cyclopropyl-2H-1,2,3-triazol-4-yl)-7-(4,4,5,5-tetramethyl-1,3,2-dioxaborolan-2-yl)-1-((2-(trimethylsilyl)ethoxy)methyl)-1H-indazole (125.0 mg, 0.166 mmol, 1.0 equiv) and tert-butyl (1R,3s,5S)-3-((6-iodopyridazin-3-yl)(methyl)amino)-8-azabicyclo[3.2.1]octane-8-carboxylate (73.8 mg, 0.166 mmol, 1 equiv) in dioxane/H₂O (1.2 mL/0.12 mL) were added K₃PO₄ (105.8 mg, 0.498 mmol, 3.0 equiv) and Pd(dppf)Cl₂ (12.2 mg, 0.017 mmol, 0.1 equiv) in portions at room temperature under nitrogen atmosphere. The resulting mixture was stirred for 1 hr at 80° C. under a nitrogen atmosphere. The resulting mixture was concentrated under reduced pressure. The residue was purified by silica gel column chromatography, eluted with petroleum ether/ethyl acetate (1:1) to afford tert-butyl (1R,3s,5S)-3-((6-(4-(2-cyclopropyl-2H-1,2,3-triazol-4-yl)-1-((2-(trimethylsilyl)ethoxy)methyl)-1H-indazol-7-yl)pyridazin-3-yl)(methyl)amino)-8-azabicyclo[3.2.1]octane-8-carboxylate (40 mg, C3) as an oil that was taken to the next step without further purification. LCMS (ES, m/z): 672 [M+H]⁺

### Synthesis of Compound 312

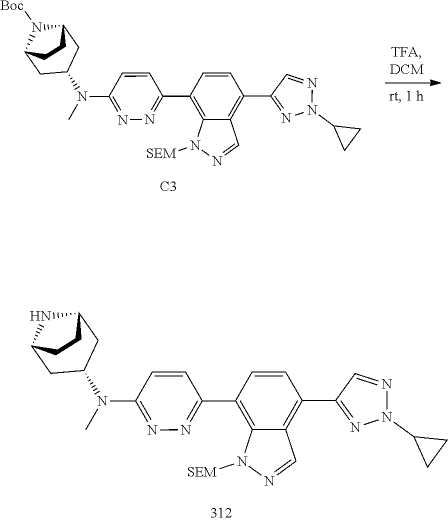

To a stirred solution of tert-butyl (1R,3s,5S)-3-((6-(4-(2-cyclopropyl-2H-1,2,3-triazol-4-yl)-1-((2-(trimethylsilyl)ethoxy)methyl)-1H-indazol-7-yl)pyridazin-3-yl)(methyl)amino)-8-azabicyclo[3.2.1]octane-8-carboxylate (45.0 mg, 0.067 mmol, 1.0 equiv) in dichloromethane (1.0 mL) was added trifluoroacetic acid (0.1 mL) dropwise portions at room temperature. The resulting mixture was stirred for 2 hr at room temperature. The resulting mixture was concentrated under reduced pressure. The crude product was purified by Prep-HPLC with the following conditions (NH₃H₂O) to afford (1R,3s,5S)-N-(6-(4-(2-cyclopropyl-2H-1,2,3-triazol-4-yl)-1H-indazol-7-yl)pyridazin-3-yl)-N-methyl-8-azabicyclo[3.2.1]octan-3-amine (10.3 mg, Compound 312) as a solid. LCMS (ES, m/z): 442 [M+H]⁺¹H NMR (400 MHz, DMSO-d₆) δ 13.20 (s, 1H), 8.75-8.72 (m, 1H), 8.66-8.63 (m, 1H), 8.51 (s, 1H), 8.28 (d, J=9.8 Hz, 1H), 8.03 (d, J=7.7 Hz, 1H), 7.76 (d, J=7.6 Hz, 1H), 7.42 (d, J=9.8 Hz, 1H), 5.20 (td, J=11.3, 5.7 Hz, 1H), 4.29 (tt, J=7.5, 3.8 Hz, 1H), 4.20-4.09 (m, 2H), 3.03 (s, 3H), 2.24-2.12 (m, 2H), 2.10-2.05 (m, 4H), 1.91-1.75 (m, 2H), 1.35 (p, J=4.9, 4.4 Hz, 2H), 1.19 (td, J=7.6, 5.1 Hz, 2H).

### Example 70: Synthesis of Compound 313

### Synthesis of Intermediate C4

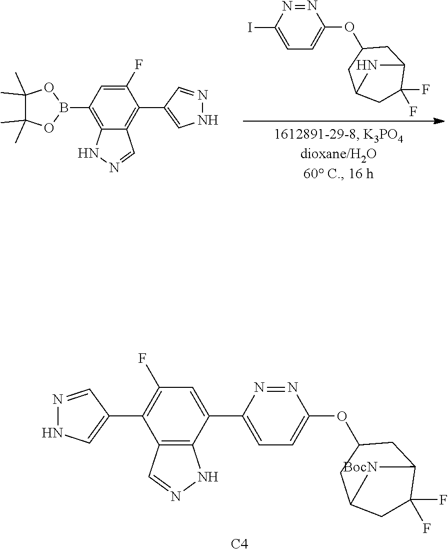 [SMILES: CC1(C)OB(c2cc(F)c(-c3cn[nH]c3)c3cn[nH]c23)OC1(C)C ; FC1(F)CC2CC(Oc3ccc(I)nn3)CC1N2 ; CC(C)(C)OC(=O)N1C2CC(Oc3ccc(-c4cc(F)c(-c5cn[nH]c5)c5cn[nH]c45)nn3)CC1C(F)(F)C2 ; "C4"]

To a stirred solution of 5-fluoro-4-(1H-pyrazol-4-yl)-7-(4,4,5,5-tetramethyl-1,3,2-dioxaborolan-2-yl)-1H-indazole (40 mg, 0.122 mmol, 1.00 eq.) and tert-butyl 6,6-difluoro-3-[(6-iodopyridazin-3-yl)oxy]-8-azabicycleo [3.2.1]octane-8-carboxylate (63 mg, 0.134 mmol, 1.1 eq.) in dioxane (4 mL) and water (0.8 mL) were added tripotassium phosphate (78 mg, 0.366 mmol, 3 eq.) and Pd-PEPPSI-IPentCl 2-methylpyridine (o-picoline (5 mg, 0.006 mmol, 0.2 eq.) dropwise for 16 hr at 80° C. under a nitrogen atmosphere. The mixture was allowed to cool to 25° C. The resulting mixture was extracted with ethyl acetate (2×10 mL). The combined organic layers were washed with water (3×15 mL) and dried over anhydrous sodium sulfate. After filtration, the filtrate was concentrated under reduced pressure. The residue was purified by silica gel column chromatography, eluted with petroleum ether/ethyl acetate (1:3) to afford tert-butyl 6,6-difluoro-3-({6-[5-fluoro-4-(1H-pyrazol-4-yl)-1H-indazol-7-yl] pyridazin-3-yl} oxy)-8-azabicyclo [3.2.1] octane-8-carb oxy late (30 mg, C4) as a solid that was taken to the next step without further purification. LCMS (ES, m/z): 542 [M+H]

### Synthesis of Compound 313

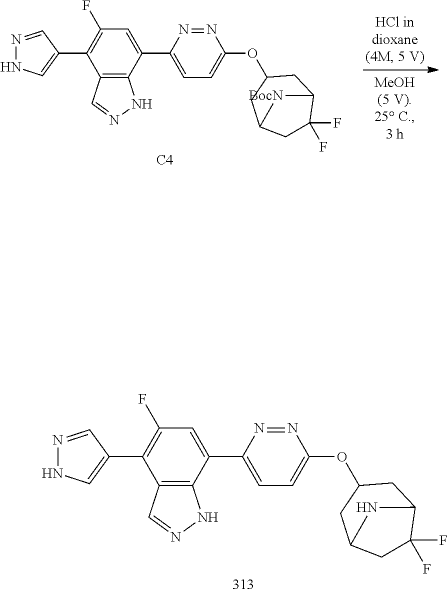

A solution of tert-butyl 6,6-difluoro-3-({6-[5-fluoro-4-(1H-pyrazol-4-yl)-1H-indazol-7-yl]pyridazin-3-yl} oxy)-8-azabicyclo[3.2.1]octane-8-carboxylate (30 mg, 0.055 mmol, 1.00 eq.) and hydrochloric acid (gas) in 1,4-dioxane (4M, 6 mL) in methanol (6 mL) was stirred for 3 hr at 25° C. The resulting mixture was concentrated under reduced pressure. The crude product was purified by preparative HPLC (Condition 4, Gradient 1) to afford 7-[6-({6,6-difluoro-8-azabicyclo[3.2.1]octan-3-yl}oxy) pyridazin-3-yl]-5-fluoro-4-(1H-pyrazol-4-yl)-1H-indazole (7.3 mg, Compound 313) as a solid. LCMS (ES, m/z): 442 [M+H] ¹H NMR (400 MHz, DMSO-d₆) δ 13.36 (s, 1H), 13.29 (s, 1H), 8.53 (d, J=1.5 Hz, 1H), 8.46 (d, J=9.0 Hz, 2H), 8.17 (s, 1H), 8.06 (d, J=12.4 Hz, 1H), 7.37 (d, J=9.4 Hz, 1H), 5.75 (dd, J=11.3, 5.7 Hz, 1H), 3.67 (d, J=7.4 Hz, 1H), 3.53 (d, J=12.8 Hz, 1H), 2.98 (s, 1H), 2.37 (s, 2H), 2.24 (q, J=13.4 Hz, 1H), 1.78 (d, J=11.1 Hz, 2H). ¹⁹F NMR (376 MHz, DMSO-d₆) δ-85.76,-110.67, -125.09.

### Example 71: Synthesis of Compound 315

### Synthesis of Intermediates C5 and C6

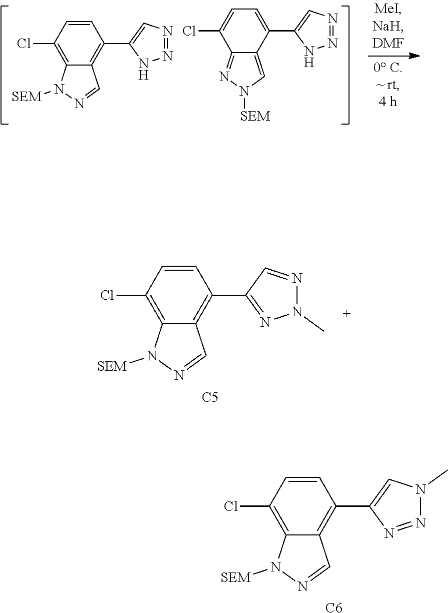

To a solution of 7-chloro-4-(1H-1,2,3-triazol-5-yl)-1-((2-(trimethylsilyl)ethoxy)methyl)-1H-indazole (1.0 g, 2.858 mmol, 1 equiv) in dimethylformamide (10 mL) was added NaH (0.2 g, 4.287 mmol, 1.5 equiv, 60% in oil) at 0° C. The mixture was stirred for 15 min. Then to the mixture was added methyl iodide (0.5 g, 3.430 mmol, 1.2 equiv). The mixture was allowed to warm to room temperature and stirred for 1 hr. The reaction mixture was quenched by water (10 mL) and extracted with ethyl acetate (3×25 mL). The combined organic layers were washed with water (1×50 mL) and brine (1×50 mL), dried over anhydrous Na₂SO₄. After filtration, the filtrate was concentrated under reduced pressure. The residue was purified by silica gel column chromatography, eluted with petroleum ether/ethyl acetate (3:1) to afford 7-chloro-4-(2-methyl-2H-1,2,3-triazol-4-yl)-1-((2-(trimethylsilyl)ethoxy)methyl)-1H-indazole (546 mg, C5) and eluted with dichloromethane/methanol (10/1) to afford 7-chloro-4-(1-methyl-1H-1,2,3-triazol-4-yl)-1-((2-(trimethylsilyl)ethoxy)methyl)-1H-indazole (346 mg, C6) as solids that were taken to the next step without further purification LCMS (ES, m/z): 364 [M+H]⁺

### Synthesis of Intermediate C7

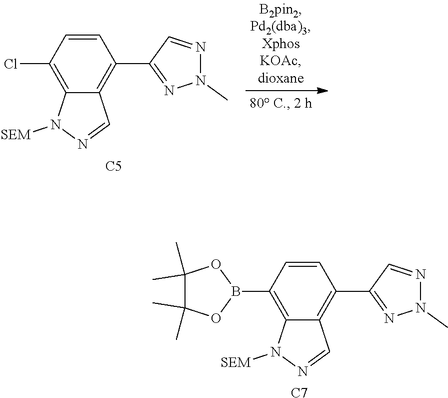

To a stirred mixture of 7-chloro-4-(2-methyl-2H-1,2,3-triazol-4-yl)-1-((2-(trimethylsilyl)ethoxy)methyl)-1H-indazole (600 mg, 1.649 mmol, 1.00 equiv), potassium acetate (485 mg, 4.947 mmol, 3 equiv) and bis(pinacolato)diboron (544 mg, 2.144 mmol, 1.3 equiv) in dioxane (12 mL) was added X-Phos (79 mg, 0.165 mmol, 0.1 equiv) and Pd₂(dba)₃ (95 mg, 0.165 mmol, 0.1 equiv) at room temperature under a nitrogen atmosphere. The resulting mixture was stirred for 2 hr at 80° C. under a nitrogen atmosphere. The resulting mixture was concentrated under vacuum. The residue was purified by silica gel column chromatography, eluted with PE/EA (1:1) to afford 4-(2-methyl-2H-1,2,3-triazol-4-yl)-7-(4,4,5,5-tetramethyl-1,3,2-dioxaborolan-2-yl)-1-((2-(trimethylsilyl)ethoxy)methyl)-1H-indazole (420 mg, C7) as a solid that was taken to the next step without further purification. LCMS (ES, m/z): 456 [M+H]⁺

### Synthesis of Intermediate C8

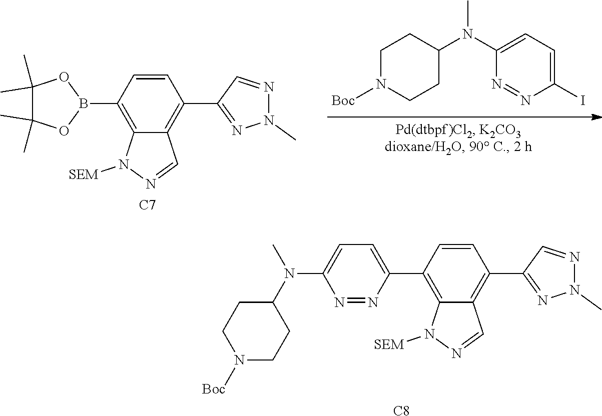

To a stirred mixture of 4-(2-methyl-2H-1,2,3-triazol-4-yl)-7-(4,4,5,5-tetramethyl-1,3,2-dioxaborolan-2-yl)-1-((2-(trimethylsilyl)ethoxy)methyl)-1H-indazole (70 mg, 0.154 mmol, 1.00 equiv), K₃PO₄ (65 mg, 0.308 mmol, 2 equiv) and tert-butyl 4-[(6-iodopyridazin-3-yl)(methyl)amino]piperidine-1-carboxylate (58 mg, 0.139 mmol, 0.9 equiv) in H₂O (0.4 mL) and dioxane (2 mL) were added Pd(dppf)Cl₂·CH₂Cl₂ (6 mg, 0.008 mmol, 0.05 equiv) at room temperature under nitrogen atmosphere. The resulting mixture was stirred for 1 hr at 90° C. under nitrogen atmosphere. The resulting mixture was concentrated under vacuum. The residue was purified by silica gel column chromatography, eluted with petroleum ether/ethyl acetate (1:1) to afford tert-butyl 4-(methyl (6-(4-(2-methyl-2H-1,2,3-triazol-4-yl)-1-((2-(trimethylsilyl)ethoxy)methyl)-1H-indazol-7-yl)pyridazin-3-yl)amino)piperidine-1-carboxylate (40 mg, C8) as a solid that was taken to the next step without further purification. LCMS (ES, m/z): 620 [M+H]⁺

### Synthesis of Compound 315

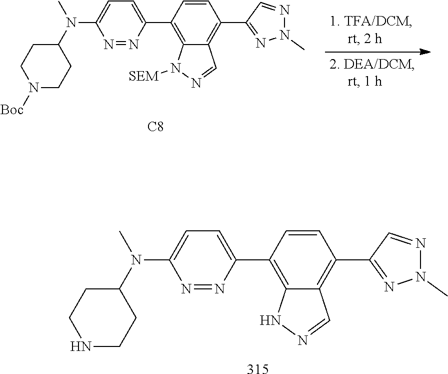

A solution of tert-butyl 4-[methyl({6-[4-(2-methyl-1,2,3-triazol-4-yl)-1-{[2-(trimethylsilyl)ethoxy] methyl}indazol-7-yl]pyridazin-3-yl})amino]piperidine-1-carboxylate (70 mg, 0.113 mmol, 1.00 equiv) in dichloromethane (2 mL) and trifluoroacetic acid (0.5 mL) was stirred for 1 hr at room temperature. The resulting mixture was concentrated under vacuum. The residue was dissolved in dichloromethane (1 mL), followed by the addition of ethylenediamine (135.74 mg, 2.260 mmol, 20 equiv). The resulting mixture was stirred for 1 hr at room temperature. The resulting mixture was concentrated under vacuum. The residue was purified by Prepparative HPLC (Condition 13, Gradient 1) to afford N-methyl-6-[4-(2-methyl-1,2,3-triazol-4-yl)-1H-indazol-7-yl]-N-(piperidin-4-yl)pyridazin-3-amine (9.7 mg, Compound 315) as a solid. LCMS (ES, m/z): 390 [M+H]⁺¹H NMR (400 MHz, Methanol-d₄) δ 8.85 (s, 1H), 8.76 (d, J=10.0 Hz, 1H), 8.32 (d, J=15.4 Hz, 2H), 8.21 (d, J=7.7 Hz, 1H), 7.84 (d, J=7.7 Hz, 1H), 4.77 (s, 1H), 4.36 (s, 3H), 3.64 (d, J=12.8 Hz, 2H), 3.42-3.34 (m, 4H), 2.38-2.26 (m, 2H), 2.20 (m, 2H).

### Example 72: Synthesis of Compound 317

### Synthesis of Intermediate C9

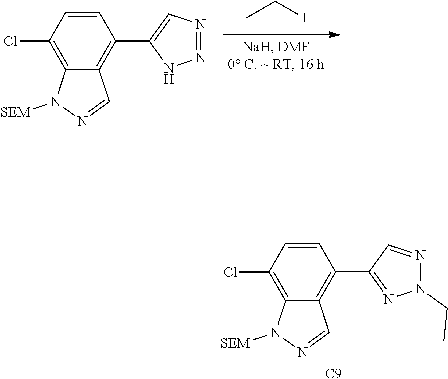

To a stirred mixture of 7-chloro-4-(3H-1,2,3-triazol-4-yl)-1-{[2-(trimethylsilyl)ethoxy]methyl}indazole (500 mg, 1.429 mmol, 1.00 equiv) in DMF (10 mL) was added 60% NaH (86 mg, 2.143 mmol, 1.5 equiv) and ethyl iodide (334 mg, 2.143 mmol, 1.5 equiv) at room temperature under N₂ atmosphere. The resulting mixture was stirred for 1 hr at room temperature under N₂ atmosphere. The reaction was quenched with saturated NH₄Cl (10 mL) at room temperature. The resulting mixture was extracted with ethyl acetate (10 mL×3). The combined organic layers were washed with H₂O and brine and dried over anhydrous Na₂SO₄. After filtration, the filtrate was concentrated under reduced pressure. The residue was purified by silica gel column chromatography, eluted with PE/EA (2/1) to afford 7-chloro-4-(2-ethyl-1,2,3-triazol-4-yl)-1-{[2-(trimethylsilyl)ethoxy]methyl}indazole (200 mg, C9) as a solid that was taken to the next step without further purification. LCMS (ES, m/z): 378 [M+H]⁺

### Synthesis of Intermediate C10

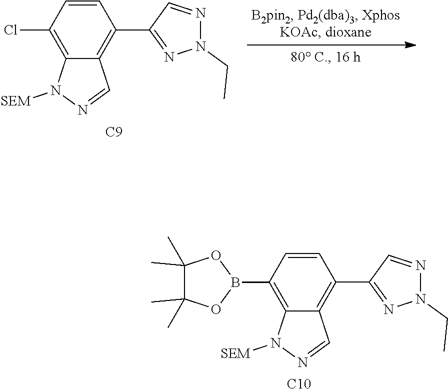 [SMILES: CCn1ncc(-c2ccc(Cl)c3c2cnn3COCC[Si](C)(C)C)n1 ; "C9" ; CCn1ncc(-c2ccc(B3OC(C)(C)C(C)(C)O3)c3c2cnn3COCC[Si](C)(C)C)n1 ; "C10"]

To a stirred solution of 7-chloro-4-(2-ethyl-1,2,3-triazol-4-yl)-1-{[2-(trimethylsilyl)ethoxy]methyl} indazole (140 mg, 0.370 mmol, 1.00 equiv) and B₂Pin₂ (141 mg, 0.555 mmol, 1.5 equiv) in dioxane (3 mL) were added Xphos (18 mg, 0.037 mmol, 0.1 equiv) and Pd₂(dba)₃ (34 mg, 0.037 mmol, 0.1 equiv) under N₂ atmosphere. The resulting mixture was stirred for 16 h at 80° C. under N₂ atmosphere. The resulting mixture was concentrated under reduced pressure. The residue was purified by silica gel column chromatography, eluted with DCM/MeOH (10/1) to afford 4-(2-ethyl-1,2,3-triazol-4-yl)-7-(4,4,5,5-tetramethyl-1,3,2-dioxaborolan-2-yl)-1-{[2-(trimethylsilyl)ethoxy]methyl}indazole (120 mg, crude) as a solid that was taken to the next step without further purification. LCMS (ES, m/z): 470 [M+H]⁺

### Synthesis of Intermediate C11

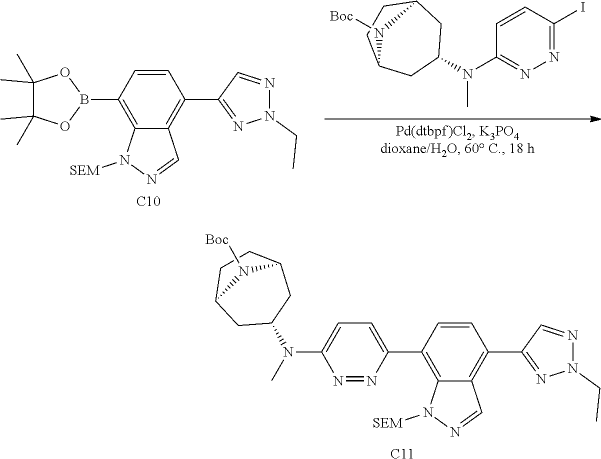 [SMILES: CCn1ncc(-c2ccc(B3OC(C)(C)C(C)(C)O3)c3c2cnn3COCC[Si](C)(C)C)n1 ; CN(c1ccc(I)nn1)[C@@H]1C[C@H]2CC[C@@H](C1)N2C(=O)OC(C)(C)C ; "C10" ; CCn1ncc(-c2ccc(-c3ccc(N(C)[C@@H]4C[C@H]5CC[C@@H](C4)N5C(=O)OC(C)(C)C)nn3)c3c2cnn3COCC[Si](C)(C)C)n1 ; "C11"]

To a stirred solution of 4-(2-ethyl-1,2,3-triazol-4-yl)-7-(4,4,5,5-tetramethyl-1,3,2-dioxaborolan-2-yl)-1-{[2-(trimethylsilyl)ethoxy]methyl}indazole (120 mg crude, 0.107 mmol, 1.00 equiv) and tert-butyl 3-[(6-iodopyridazin-3-yl)(methyl)amino]-8-azabicyclo[3.2.1]octane-8-carboxylate (48 mg, 0.107 mmol, 1.0 equiv) in dioxane (1 mL) and H₂O (1 mL) were added K₂CO₃ (68 mg, 0.493 mmol, 4.60 equiv) and Pd(dppf)Cl₂·CH₂Cl₂ (9 mg, 0.011 mmol, 0.1 equiv) under N2 atmosphere. The resulting mixture was stirred for 1 hr at 80° C. under N₂ atmosphere. The resulting mixture was concentrated under reduced pressure. The residue was purified by silica gel column chromatography, eluted with PE/EA (1:1) to afford tert-butyl 3-({6-[4-(2-ethyl-1,2,3-triazol-4-yl)-1-{[2-(trimethylsilyl)ethoxy]methyl}indazol-7-yl]pyridazin-3-yl}(methyl)amino)-8-azabicyclo[3.2.1]octane-8-carboxylate (30 mg, C11) as a solid that was taken to the next step without further purification. LCMS (ES, m/z): 660 [M+H]⁺

### Synthesis of Compound 317

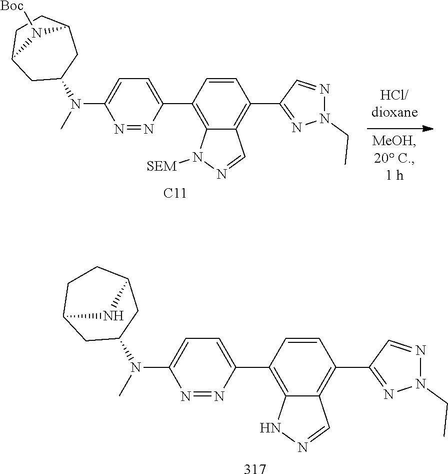

To a stirred mixture of tert-butyl 3-({6-[4-(2-ethyl-1,2,3-triazol-4-yl)-1-{[2-(trimethylsilyl)ethoxy] methyl}indazol-7-yl]pyridazin-3-yl}(methyl)amino)-8-azabicyclo[3.2.1]octane-8-carboxylate (30 mg, 0.045 mmol, 1.00 equiv) in DCM (1 mL) was added TFA (0.3 mL). The mixture was stirred for 3 hr at 20° C. under N₂ atmosphere. The resulting mixture was concentrated under vacuum. To the above mixture was added 1,2-ethylenediamine (0.01 mL) and DCM (1 mL). The resulting mixture was stirred for 1h at room temperature under N₂ atmosphere. The resulting mixture was concentrated under reduced pressure. The crude product was purified by Prep-HPLC (Condition 13, Gradient 2) to afford N-{6-[4-(2-ethyl-1,2,3-triazol-4-yl)-1H-indazol-7-yl]pyridazin-3-yl}-N-methyl-8-azabicyclo[3.2.1]octan-3-amine (14.2 mg, Compound 317) as a solid. LCMS (ES, m/z): 430 [M+H]+ 1H NMR (400 MHz, Methanol-d₄) δ 8.88 (s, 1H), 8.75 (d, J=10.0 Hz, 1H), 8.35 (s, 1H), 8.29-8.19 (m, 2H), 7.87 (d, J=7.7 Hz, 1H), 4.65 (q, J=7.4 Hz, 2H), 4.29 (s, 2H), 2.46-2.42 (m, 2H), 2.38-2.34 (m, 2H), 2.28 (s, 1H), 2.14 (d, J=13.8 Hz, 2H), 1.68 (t, J=7.4 Hz, 3H).

### Example 73: Synthesis of Compound 318

### Synthesis of Intermediate C12

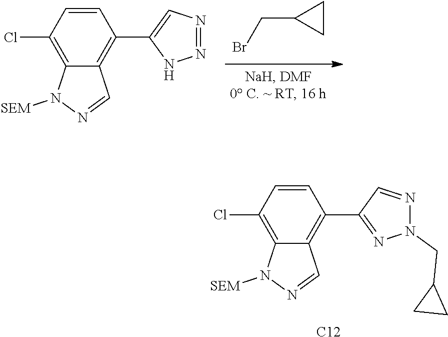

To a stirred mixture of 7-chloro-4-(3H-1,2,3-triazol-4-yl)-2-{[2-(trimethylsilyl)ethoxy]methyl}indazole (500 mg, 1.429 mmol, 1.00 equiv) in DMF (10 mL) was added NaH (86 mg, 2.143 mmol, 1.5 equiv) and (bromomethyl)cyclopropane (289 mg, 2.143 mmol, 1.5 equiv) at room temperature under N₂ atmosphere. The resulting mixture was stirred for 1 h at room temperature under N₂ atmosphere. The reaction was quenched with saturated NH₄Cl (10 mL) at room temperature. The resulting mixture was extracted with ethyl acetate (10 mL×3). The combined organic layers were washed with H₂O and brine and dried over anhydrous Na₂SO₄. After filtration, the filtrate was concentrated under reduced pressure. The residue was purified by silica gel column chromatography, eluted with PE/EA (2:1) to afford 7-chloro-4-[2-(cyclopropylmethyl)-1,2,3-triazol-4-yl]-1-{[2-(trimethylsilyl)ethoxy]methyl}indazole (190 mg, C12) as a solid that was taken to the next step without further purification. LCMS (ES, m/z): 404 [M+H]⁺

### Synthesis of Intermediate C13

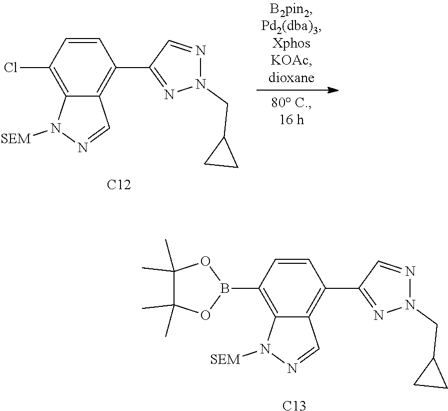 [SMILES: C[Si](C)(C)CCOCn1ncc2c(-c3cnn(CC4CC4)n3)ccc(Cl)c21 ; "C12" ; CC1(C)OB(c2ccc(-c3cnn(CC4CC4)n3)c3cnn(COCC[Si](C)(C)C)c23)OC1(C)C ; "C13"]

To a stirred mixture of 7-chloro-4-[2-(cyclopropylmethyl)-1,2,3-triazol-4-yl]-1-{[2-(trimethylsilyl) ethoxy]methyl}indazole (125 mg, 0.309 mmol, 1.00 equiv), potassium acetate (61 mg, 0.618 mmol, 2 equiv) and bis(pinacolato)diboron (118 mg, 0.464 mmol, 1.5 equiv) in dioxane (2 mL) was added Xphos (15 mg, 0.031 mmol, 0.1 equiv) and Pd₂(dba)₃ (28 mg, 0.031 mmol, 0.1 equiv) at room temperature under nitrogen atmosphere. The resulting mixture was stirred for 2 hr at 80° C. under nitrogen atmosphere. The resulting mixture was concentrated under vacuum. The residue was purified by silica gel column chromatography, eluted with PE/EA (2:1) to afford 4-[2-(cyclopropylmethyl)-1,2,3-triazol-4-yl]-7-(4,4,5,5-tetramethyl-1,3,2-dioxaborolan-2-yl)-1-{[2-(trimethylsilyl)ethoxy]methyl}indazole (120 mg, C13) as a solid that was taken to the next step without further purification. LCMS (ES, m/z): 496 [M+H]⁺

### Synthesis of Intermediate C14

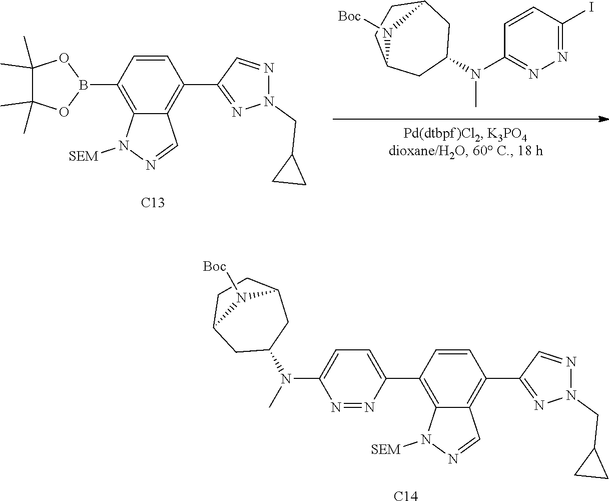 [SMILES: CC1(C)OB(c2ccc(-c3cnn(CC4CC4)n3)c3cnn(COCC[Si](C)(C)C)c23)OC1(C)C ; CN(c1ccc(I)nn1)[C@@H]1C[C@H]2CC[C@@H](C1)N2C(=O)OC(C)(C)C ; "C13" ; CN(c1ccc(-c2ccc(-c3cnn(CC4CC4)n3)c3cnn(COCC[Si](C)(C)C)c23)nn1)[C@@H]1C[C@H]2CC[C@@H](C1)N2C(=O)OC(C)(C)C ; "C14"]

To a stirred solution of 4-[2-(cyclopropylmethyl)-1,2,3-triazol-4-yl]-7-(4,4,5,5-tetramethyl-1,3,2-dioxaborolan-2-yl)-1-{[2-(trimethylsilyl)ethoxy]methyl}indazole (120 mg crude, 0.073 mmol, 1.0 equiv) and tert-butyl 3-[(6-iodopyridazin-3-yl)(methyl)amino]-8-azabicyclo[3.2.1]octane-8-carboxylate (32 mg, 0.073 mmol, 1.00 equiv) in dioxane (2 mL) and H₂O (0.4 mL) were added K₂CO₃ (30 mg, 0.219 mmol, 3.0 equiv) and Pd(dppf)Cl₂·CH₂Cl₂ (6 mg, 0.007 mmol, 0.10 equiv) at room temperature under N₂ atmosphere. The resulting mixture was stirred for 1 h at 80° C. under N₂ atmosphere. The resulting mixture was concentrated under reduced pressure. The residue was purified by silica gel column chromatography, eluted with PE/EA (1/1) to afford tert-butyl 3-[(6-{4-[2-(cyclopropylmethyl)-1,2,3-triazol-4-yl]-1-{[2-(trimethyl silyl)ethoxy]methyl}indazol-7-yl}pyridazin-3-yl)(methyl)amino]-8-azabicyclo[3.2.1]octane-8-carboxylate (20 mg, C14) as a solid that was taken to the next step without further purification. LCMS (ES, m/z): 708 [M+Na]⁺

### Synthesis of Compound 318

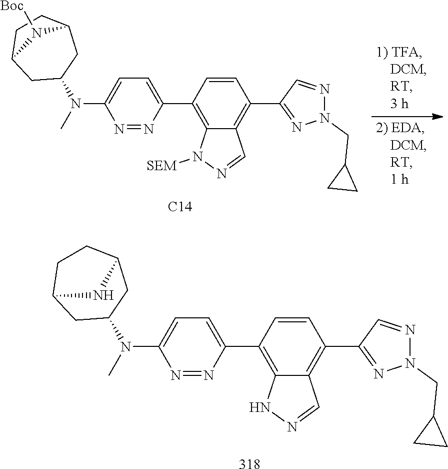 [SMILES: CN(c1ccc(-c2ccc(-c3cnn(CC4CC4)n3)c3cnn(COCC[Si](C)(C)C)c23)nn1)[C@@H]1C[C@H]2CC[C@@H](C1)N2C(=O)OC(C)(C)C ; "C14" ; CN(c1ccc(-c2ccc(-c3cnn(CC4CC4)n3)c3cn[nH]c23)nn1)[C@@H]1C[C@H]2CC[C@@H](C1)N2 ; "318"]

To a stirred mixture of tert-butyl 3-[(6-{4-[2-(cyclopropylmethyl)-1,2,3-triazol-4-yl]-1-{[2-(trimethyl silyl)ethoxy]methyl}indazol-7-yl}pyridazin-3-yl)(methyl)amino]-8-azabicyclo[3.2.1]octane-8-carboxylate (20 mg, 0.029 mmol, 1.00 equiv) in DCM (0.5 mL) was added TFA (0.1 mL). The mixture was stirred for 3 hr at room temperature under N₂ atmosphere. The resulting mixture was concentrated under vacuum. To the above mixture was added 1,2-ethylenediamine (0.01 mL). The resulting mixture was stirred for 1 h at room temperature under N₂ atmosphere. The resulting mixture was concentrated under reduced pressure. The crude product was purified by preparative HPLC (Condition 13, Gradient 2) to afford N-(6-{4-[2-(cyclopropylmethyl)-1,2,3-triazol-4-yl]-1H-indazol-7-yl}pyridazin-3-yl)-N-methyl-8-azabicyclo[3.2.1]octan-3-amine (5.0 mg, Compound 318) as a solid. LCMS (ES, m/z): 456 [M+H]+ 1H NMR (400 MHz, Methanol-d₄) δ 8.89 (s, 1H), 8.75 (d, J=10.0 Hz, 1H), 8.36 (s, 1H), 8.24 (m 2H), 7.87 (d, J=7.7 Hz, 1H), 4.47 (d, J=7.3 Hz, 2H), 4.29 (s, 2H), 3.28 (s, 2H), 2.44 (d, J=12.6 Hz, 2H), 2.37 (d, J=10.3 Hz, 2H), 2.28 (s, 1H), 2.14 (d, J=14.1 Hz, 2H), 1.52 (s, 1H), 0.71 (d, J=7.6 Hz, 2H), 0.58 (s, 2H).

### Example 74: Synthesis of Compound 319

### Synthesis of Compound 319

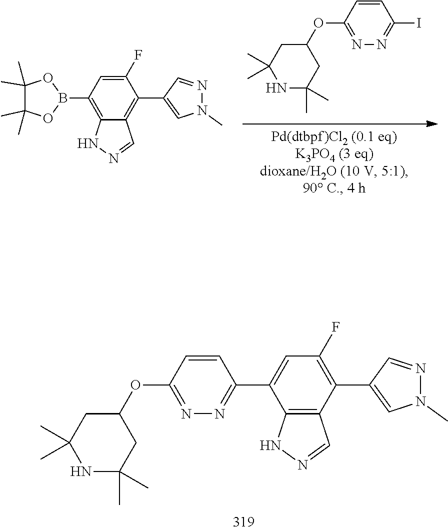

To a stirred solution of 5-fluoro-4-(1-methylpyrazol-4-yl)-7-(4,4,5,5-tetramethyl-1,3,2-dioxaborolan-2-yl)-1H-indazole (100 mg, 0.292 mmol, 1.00 eq.) and 3-iodo-6-[(2,2,6,6-tetramethylpiperidin-4-yl)oxy]pyridazine (126.68 mg, 0.350 mmol, 1.2 eq.) in dioxane (5 mL) and water (1 mL) were added Pd(dtbpf)Cl₂ (19.05 mg, 0.029 mmol, 0.1 eq.) and tripotassium phosphate (124.07 mg, 0.584 mmol, 2 eq.) dropwise for 4 hr at 90° C. under a nitrogen atmosphere. The mixture was allowed to cool down to 25° C. The resulting mixture was extracted with ethyl acetate (3×20 mL). The combined organic layers were washed with water (3×10 mL), dried over anhydrous sodium sulfate. After filtration, the filtrate was concentrated under reduced pressure. The crude product was purified by preparative HPLC with the following conditions (Condition 4, Gradient 13). This resulted in 5-fluoro-4-(1-methylpyrazol-4-yl)-7-{6-[(2,2,6,6-tetramethylpiperidin-4-yl)oxy]pyridazin-3-yl}-1H-indazole (13.1 mg, Compound 319) as a solid. LCMS (ES, m/z): 450 [M+H] ¹H NMR (400 MHz, DMSO-d₆): δ 13.30 (s, 1H), 8.53 (s, 1H), 8.45 (d, J=11.2 Hz, 2H), 8.11 (d, J=2.1 Hz, 1H), 8.06 (d, J=12.4 Hz, 1H), 7.35 (d, J=9.3 Hz, 1H), 5.85-5.75 (m, 1H), 3.99 (s, 3H), 2.13 (dd, J=11.9, 4.1 Hz, 2H), 1.40 (s, 1H), 1.31 (t, J=11.5 Hz, 2H), 1.26 (s, 6H), 1.13 (s, 6H). ¹⁹F NMR (400 MHz, DMSO-d₆) δ-125.03.

### Example 75: Synthesis of Compound 320

### Synthesis of Compound 320

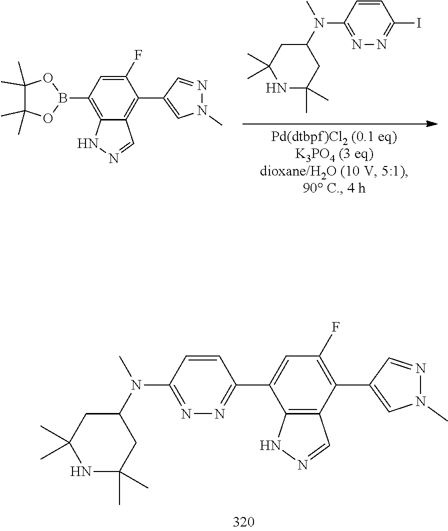

To a stirred solution of 5-fluoro-4-(1-methylpyrazol-4-yl)-7-(4,4,5,5-tetramethyl-1,3,2-dioxaborolan-2-yl)-1H-indazole (100 mg, 0.292 mmol, 1.00 equiv) and Pd(dppf)Cl₂ (21.38 mg, 0.029 mmol, 0.1 equiv) in dioxane (2 mL, 23.608 mmol, 80.78 equiv) were added 6-iodo-N-methyl-N-(2,2,6,6-tetramethylpiperidin-4-yl)pyridazin-3-amine (131.25 mg, 0.350 mmol, 1.2 equiv) and K₃PO₄ (61.98 mg, 0.876 mmol, 3 equiv) dropwise for 4 hr at 100° C. under N₂ atmosphere. The mixture was allowed to cool down to 25° C. The resulting mixture was extracted with ethyl acetate (2×10 mL). The combined organic layers were washed with H₂O (3×10 mL), dried over anhydrous Na₂SO₄. After filtration, the filtrate was concentrated under reduced pressure. The crude product was purified by preparative HPLC (Condition 4, Gradient 10). This resulted in 6-[5-fluoro-4-(1-methylpyrazol-4-yl)-1H-indazol-7-yl]-N-methyl-N-(2,2,6,6-tetramethylpiperidin-4-yl)pyridazin-3-amine (18.4 mg, Compound 320) as a solid. LCMS (ES, m/z): 463 [M+H] 1H NMR (400 MHz, DMSO-d₆) δ 13.19 (s, 1H), 8.49 (s, 1H), 8.43 (s, 1H), 8.21 (d, J=9.8 Hz, 1H), 8.08 (d, J=2.0 Hz, 1H), 7.93 (d, J=12.6 Hz, 1H), 7.26 (d, J=9.8 Hz, 1H), 5.11 (s, 1H), 3.98 (s, 3H), 3.01 (s, 3H), 1.56 (dd, J=12.0, 3.7 Hz, 2H), 1.47 (t, J=12.0 Hz, 2H), 1.29 (s, 6H), 1.11 (s, 6H). ¹⁹F NMR (376 MHz, DMSO-d₆) δ-125.04.

### Example 76: Synthesis of Compound 321

### Synthesis of Intermediate C15

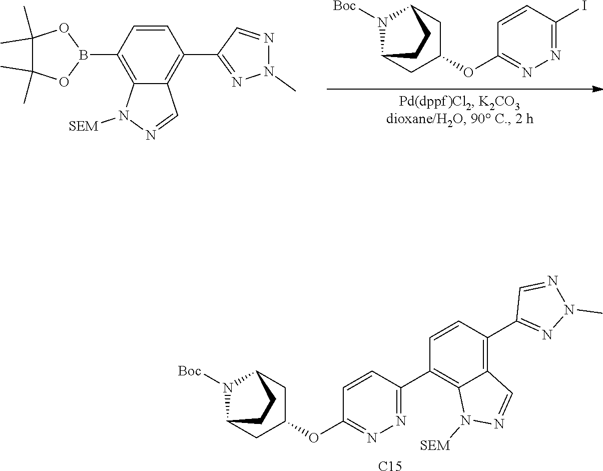

To a stirred mixture of 4-(2-methyl-2H-1,2,3-triazol-4-yl)-7-(4,4,5,5-tetramethyl-1,3,2-dioxaborolan-2-yl)-1-((2-(trimethylsilyl)ethoxy)methyl)-1H-indazole (90.0 mg, 0.198 mmol, 1.00 equiv), K₂CO₃ (54.6 mg, 0.396 mmol, 2 equiv) and tert-butyl (1R,3s,5S)-3-((6-iodopyridazin-3-yl)oxy)-8-azabicyclo[3.2.1]octane-8-carboxylate (85.2 mg, 0.198 mmol, 1 equiv) in dioxane (2 mL) and H₂O (0.4 mL) was added Pd(dppf)Cl₂·CH₂Cl₂ (8.1 mg, 0.010 mmol, 0.05 equiv) at room temperature under nitrogen atmosphere. The resulting mixture was stirred for 1 h at 90° C. under nitrogen atmosphere. The resulting mixture was concentrated under vacuum. The residue was purified by silica gel column chromatography, eluted with PE/EA (½) to afford tert-butyl (1R,3s,5S)-3-((6-(4-(2-methyl-2H-1,2,3-triazol-4-yl)-1-((2-(trimethylsilyl) ethoxy)methyl)-1H-indazol-7-yl)pyridazin-3-yl)oxy)-8-azabicyclo[3.2.1]octane-8-carboxylate (70.0 mg, C15) as a solid that was taken to the next step without further purification. LCMS (ES, m/z): 633[M+H]⁺

### Synthesis of Compound 321

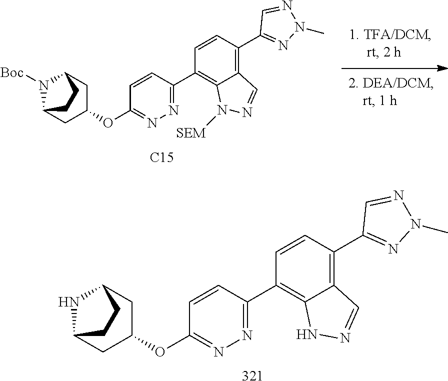

A solution of tert-butyl tert-butyl (1R,3s,5S)-3-((6-(4-(2-methyl-2H-1,2,3-triazol-4-yl)-1-((2-(tri-methylsilyl)ethoxy)methyl)-1H-indazol-7-yl)pyridazin-3-yl)oxy)-8-azabicyclo[3.2.1]octane-8-carboxylate (70.0 mg, 0.111 mmol, 1.00 equiv) in DCM (2 mL) and TFA (0.4 mL) was stirred for 1 h at room temperature. The resulting mixture was concentrated under vacuum. The residue was dissolved in DCM (1.0 mL) followed by the addition of ethylenediamine (133.0 mg, 2.220 mmol, 20 equiv). The mixture was stirred for 1 h at room temperature. The resulting mixture was concentrated under vacuum. The residue was purified by preparative HPLC (Condition 13, Gradient 2) to afford 7-{6-[(1R,3S,5S)-8-azabicyclo[3.2.1]octan-3-yloxy]pyridazin-3-yl}-4-(2-methyl-1,2,3-triazol-4-yl)-1H-indazole (10.6 mg, Compound 321) as a solid. LCMS (ES, m/z): 403 [M+H]⁺¹H NMR (400 MHz, Methanol-d4) δ 8.82 (s, 1H), 8.47 (d, J=9.4 Hz, 1H), 8.30 (s, 1H), 8.12 (d, J=7.7 Hz, 1H), 7.80 (d, J=7.6 Hz, 1H), 7.37 (d, J=9.4 Hz, 1H), 5.81 (m, 1H), 4.35 (s, 3H), 4.26 (s, 2H), 2.72-2.62 (m, 2H), 2.33-2.22 (m, 4H), 2.09 (m, 2H).

### Example 77: Synthesis of Compound 322

### Synthesis of Intermediate C16

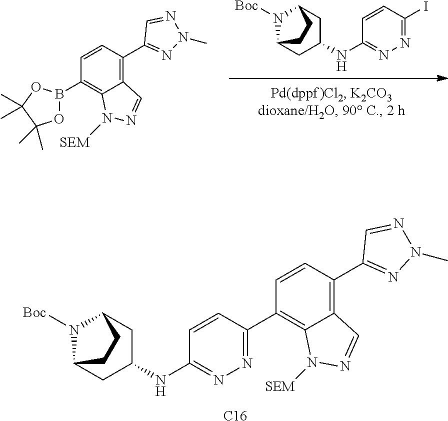

To a stirred mixture of 4-(2-methyl-2H-1,2,3-triazol-4-yl)-7-(4,4,5,5-tetramethyl-1,3,2-dioxaborolan-2-yl)-1-((2-(trimethylsilyl)ethoxy)methyl)-1H-indazole (70.0 mg, 0.154 mmol, 1.00 equiv), K₂CO₃ (42.5 mg, 0.308 mmol, 2 equiv) and tert-butyl (1R,3s,5S)-3-((6-iodopyridazin-3-yl)amino)-8-azabicyclo[3.2.1]octane-8-carboxylate (59.5 mg, 0.139 mmol, 0.9 equiv) in dioxane (2 mL) and H₂O (0.4 mL) was added Pd(dppf)Cl₂·CH₂Cl₂ (6.3 mg, 0.008 mmol, 0.05 equiv) at room temperature under nitrogen atmosphere. The resulting mixture was stirred for 2 hr at 90° C. under nitrogen atmosphere. The resulting mixture was concentrated under vacuum. The residue was purified by silica gel column chromatography, eluted with PE/EA (1:1) to afford tert-butyl (1R,3s,5S)-3-((6-(4-(2-methyl-2H-1,2,3-triazol-4-yl)-1-((2-(trimethylsilyl) ethoxy)methyl)-1H-indazol-7-yl)pyridazin-3-yl)amino)-8-azabicyclo[3.2.1]octane-8-carboxylate (43.0 mg, C16) as a solid that was taken to the next step without further purification. LCMS (ES, m/z): 632[M+H]⁺

### Synthesis of Compound 322

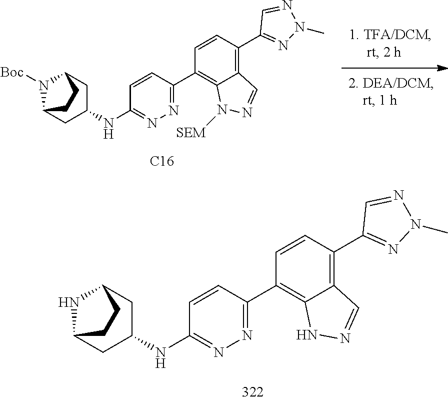

A solution of tert-butyl (1R,3s,5S)-3-((6-(4-(2-methyl-2H-1,2,3-triazol-4-yl)-1-((2-(trimethylsilyl) ethoxy)methyl)-1H-indazol-7-yl)pyridazin-3-yl)amino)-8-azabicyclo[3.2.1]octane-8-carboxylate (43.0 mg, 0.068 mmol, 1.00 equiv) in DCM (1.0 mL) and TFA (0.3 mL) was stirred for 2 hr at room temperature. The resulting mixture was concentrated under vacuum. The residue was dissolved in DCM (1.0 mL) followed by the addition of ethylenediamine (81.8 mg, 1.360 mmol, 20 equiv). The mixture was stirred for 1 h at room temperature. The resulting mixture was concentrated under vacuum. The residue was purified by preparative HPLC (Condition 13, Gradient 2) to afford 6-[4-(2-methyl-1,2,3-triazol-4-yl)-1H-indazol-7-yl]-N-{2-[(2S)-pyrrolidin-2-yl]ethyl}pyridazin-3-amine (5.5 mg, 20.75%) as a light yellow solid. LCMS (ES, m/z): 402 [M+H]⁺¹H NMR (400 MHz, DMSO-d₆) δ 8.74 (s, 1H), 8.57-8.48 (m, 2H), 8.10 (d, J=7.7 Hz, 1H), 7.81 (d, J=7.6 Hz, 1H), 7.70 (d, J=9.8 Hz, 1H), 4.34 (m, 2H), 4.29 (s, 3H), 4.13-4.06 (m, 2H), 2.55 (s, 2H), 2.22 (m, 4H), 2.07-1.99 (m, 2H), 1.93 (m, 2H).

### Example 78: Synthesis of Compound 323

### Synthesis of Intermediate C17

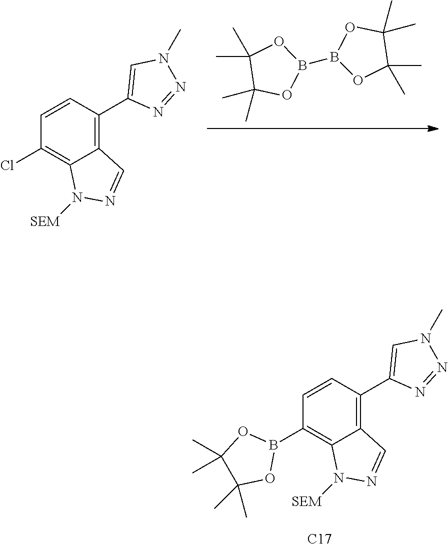

To a stirred mixture of 7-chloro-4-(1-methyl-1H-1,2,3-triazol-4-yl)-1-((2-(trimethylsilyl)ethoxy) methyl)-1H-indazole (440.0 mg, 1.209 mmol, 1.00 equiv), KOAc (356.0 mg, 3.627 mmol, 3 equiv) and bis(pinacolato)diboron (460.5 mg, 1.814 mmol, 1.5 equiv) in dioxane (8.8 mL) was added Pd(dppf)Cl₂·CH₂Cl₂ (44.2 mg, 0.060 mmol, 0.05 equiv) at room temperature under nitrogen atmosphere. The resulting mixture was stirred for 16 hr at 110° C. under nitrogen atmosphere. The resulting mixture was concentrated under vacuum. The residue was purified by silica gel column chromatography, eluted with PE/EA (1:1) to afford 4-(1-methyl-1H-1,2,3-triazol-4-yl)-7-(4,4,5,5-tetramethyl-1,3,2-dioxaborolan-2-yl)-1-((2-(trimethylsilyl)ethoxy)methyl)-1H-indazole (400.0 mg, C17) as a solid that was taken to the next step without further purification. LCMS (ES, m/z): 456[M+H]⁺

### Synthesis of Intermediate C18

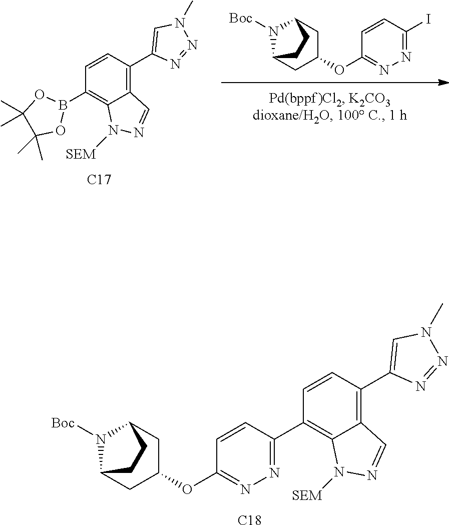

To a stirred mixture of 4-(1-methyl-1H-1,2,3-triazol-4-yl)-7-(4,4,5,5-tetramethyl-1,3,2-dioxaborolan-2-yl)-1-((2-(trimethylsilyl)ethoxy)methyl)-1H-indazole (120.0 mg, 0.263 mmol, 1.00 equiv), K₂CO₃ (109.2 mg, 0.789 mmol, 3 equiv) and tert-butyl (1R,3S,5S)-3-[(6-iodopyridazin-3-yl)oxy]-8-azabicyclo[3.2.1]octane-8-carboxylate (113.6 mg, 0.263 mmol, 1 equiv) in dioxane (2.0 mL) and H₂O (0.4 mL) was added Pd(dppf)Cl₂·CH₂Cl₂ (10.7 mg, 0.013 mmol, 0.05 equiv) at room temperature under a nitrogen atmosphere. The resulting mixture was stirred for 2 h at 90° C. under a nitrogen atmosphere. The resulting mixture was concentrated under vacuum. The residue was purified by silica gel column chromatography, eluted with ethylacetate to afford tert-butyl (1R,3s,5S)-3-((6-(4-(1-methyl-1H-1,2,3-triazol-4-yl)-1-((2-(trimethylsilyl)ethoxy)methyl)-1H-indazol-7-yl)pyridazin-3-yl)oxy)-8-azabicyclo[3.2.1]octane-8-carboxylate (60.0 mg, C18) as a solid that was taken to the next step without further purification. LCMS (ES, m/z): 633 [M+H]⁺

### Synthesis of Compound 323

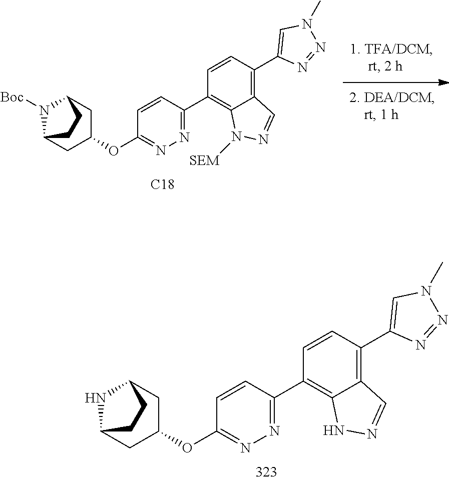

A solution of tert-butyl (1R,3s,5S)-3-((6-(4-(1-methyl-1H-1,2,3-triazol-4-yl)-1-((2-(trimethylsilyl) ethoxy)methyl)-1H-indazol-7-yl)pyridazin-3-yl)oxy)-8-azabicyclo[3.2.1]octane-8-carboxylate (60.0 mg, 0.095 mmol, 1.00 equiv) in DCM (1 mL) and TFA (0.3 mL) was stirred for 1 hr at room temperature. The resulting mixture was concentrated under vacuum. The residue was dissolved in DCM (1 mL) followed by the addition of ethylenediamine (114.0 mg, 1.9 mmol, 20 equiv). The mixture was stirred for 1 hr at room temperature. The resulting mixture was concentrated under vacuum. The residue was purified by Prep-HPLC (Condition 13, Gradient 2) to afford 7-{6-[(1R,3S,5S)-8-azabicyclo[3.2.1]octan-3-yloxy]pyridazin-3-yl}-4-(1-methyl-1,2,3-triazol-4-yl)-1H-indazole (30.8 mg, Compound 323) as a solid. LCMS (ES, m/z): 403 [M+H]+ 1H NMR (400 MHz, Methanol-d₄) δ 8.84 (s, 1H), 8.66 (s, 1H), 8.54 (d, J=9.5 Hz, 1H), 8.16 (d, J=7.7 Hz, 1H), 7.82 (d, J=7.7 Hz, 1H), 7.44 (d, J=9.4 Hz, 1H), 5.80 (m, 1H), 4.26 (d, J=4.8 Hz, 3H), 2.67 (m, 2H), 2.28 (d, J=2.5 Hz, 4H), 2.10 (m, 2H).

### Example 79: Synthesis of Compound 324

### Synthesis of Intermediate C19

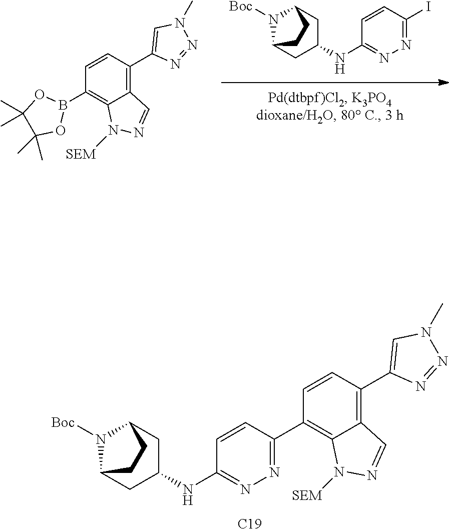

To a stirred mixture of 4-(1-methyl-1H-1,2,3-triazol-4-yl)-7-(4,4,5,5-tetramethyl-1,3,2-dioxaborolan-2-yl)-1-((2-(trimethylsilyl)ethoxy)methyl)-1H-indazole (72.0 mg, 0.158 mmol, 1.00 equiv), K₂CO₃ (65.6 mg, 0.474 mmol, 3 equiv) and tert-butyl (1R,3s,5S)-3-((6-iodopyridazin-3-yl)amino)-8-azabicyclo[3.2.1]octane-8-carboxylate (68.0 mg, 0.158 mmol, 1 equiv) in dioxane (2.0 mL) and H₂O (0.4 mL) was added Pd(dppf)Cl₂·CH₂Cl₂ (6.4 mg, 0.008 mmol, 0.05 equiv) at room temperature under nitrogen atmosphere. The resulting mixture was stirred for 1 hr at 90° C. under a nitrogen atmosphere. The resulting mixture was concentrated under vacuum. The residue was purified by silica gel column chromatography, eluted with PE/EA (1:1) to afford tert-butyl (1R,3s,5S)-3-((6-(4-(1-methyl-1H-1,2,3-triazol-4-yl)-1-((2-(trimethylsilyl) ethoxy)methyl)-1H-indazol-7-yl)pyridazin-3-yl)amino)-8-azabicyclo[3.2.1]octane-8-carboxylate (48.0 mg, C19) as a solid that was taken to the next step without further purification. LCMS (ES, m/z): 632[M+H]⁺

### Synthesis of Compound 324

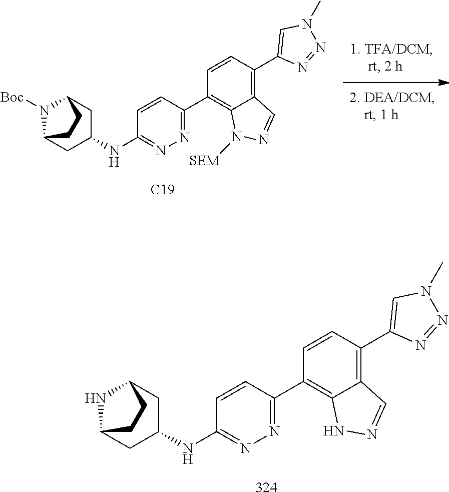

A solution of tert-butyl (1R,3s,5S)-3-((6-(4-(1-methyl-1H-1,2,3-triazol-4-yl)-1-((2-(trimethylsilyl) ethoxy)methyl)-1H-indazol-7-yl)pyridazin-3-yl)amino)-8-azabicyclo[3.2.1]octane-8-carboxylate (48.0 mg, 0.076 mmol, 1.00 equiv) in DCM (1.0 mL) and TFA (0.3 mL) was stirred for 2 hr at room temperature. The resulting mixture was concentrated under vacuum. The residue was dissolved in DCM (1.0 mL) followed by the addition of ethylenediamine (91.3 mg, 1.520 mmol, 20 equiv). The mixture was stirred for 1 hr at room temperature. The resulting mixture was concentrated under vacuum. The residue was purified by Prep-HPLC (Condition 13, Gradient 2) to afford (1R,3s,5S)-N-(6-(4-(1-methyl-1H-1,2,3-triazol-4-yl)-1H-indazol-7-yl)pyridazin-3-yl)-8-azabicyclo[3.2.1]octan-3-amine (12.0 mg, Compound 324) as a solid. LCMS (ES, m/z): 402 [M+H]⁺¹H NMR (400 MHz, Methanol-d4) δ 8.81 (s, 1H), 8.67 (s, 1H), 8.61 (d, J=9.9 Hz, 1H), 8.14 (d, J=7.7 Hz, 1H), 7.82 (d, J=7.7 Hz, 1H), 7.74 (s, 1H), 4.54 (s, 1H), 4.26 (d, J=8.9 Hz, 5H), 3.33 (m, 17H), 2.44 (d, J=11.8 Hz, 4H), 2.24 (d, J=10.1 Hz, 2H), 2.10 (m, 2H).

### Example 80: Synthesis of Compound 325

### Synthesis of Intermediate C20

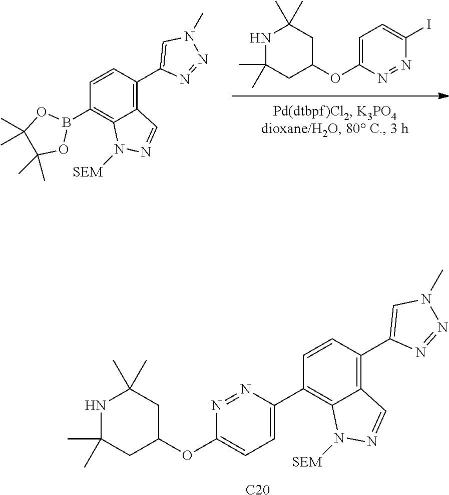

To a stirred mixture of 4-(1-methyl-1H-1,2,3-triazol-4-yl)-7-(4,4,5,5-tetramethyl-1,3,2-dioxaborolan-2-yl)-1-((2-(trimethylsilyl)ethoxy)methyl)-1H-indazole (74.0 mg, 0.162 mmol, 1.00 equiv), K₂CO₃ (67.4 mg, 0.486 mmol, 3 equiv) and 3-iodo-6-[(2,2,6,6-tetramethylpiperidin-4-yl)oxy]pyridazine (58.7 mg, 0.162 mmol, 1 equiv) in dioxane (2.0 mL) and H₂O (0.4 mL) was added Pd(dppf)Cl₂·CH₂Cl₂ (6.6 mg, 0.008 mmol, 0.05 equiv) at room temperature under nitrogen atmosphere. The resulting mixture was stirred for 1 hr at 90° C. under nitrogen atmosphere. The resulting mixture was concentrated under vacuum. The residue was purified by silica gel column chromatography, eluted with PE/EA (1:1) to afford 4-(1-methyl-1H-1,2,3-triazol-4-yl)-7-(6-((2,2,6,6-tetramethylpiperidin-4-yl)oxy)pyridazin-3-yl)-1-((2-(trimethylsilyl)ethoxy)methyl)-1H-indazole (50.0 mg, C20) as a solid that was taken to the next step without further purification. LCMS (ES, m/z): 563[M+H]⁺

### Synthesis of Compound 325

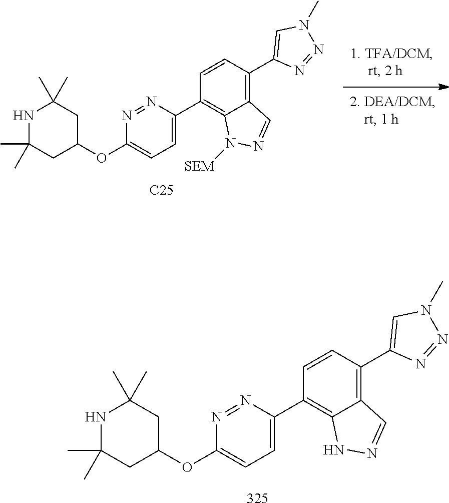

A solution of 4-(1-methyl-1H-1,2,3-triazol-4-yl)-7-(6-((2,2,6,6-tetramethylpiperidin-4-yl)oxy) pyridazin-3-yl)-1-((2-(trimethylsilyl)ethoxy)methyl)-1H-indazole (50.0 mg, 0.089 mmol, 1.00 equiv) in DCM (1.0 mL) and TFA (0.3 mL) was stirred for 2 hr at room temperature under nitrogen atmosphere. The resulting mixture was concentrated under vacuum. The residue was dissolved in DCM (1.0 mL) followed by the addition of ethylenediamine (106.8 mg, 1.780 mmol, 20 equiv). The mixture was stirred for 1 hr at room temperature. The resulting mixture was concentrated under vacuum. The residue was purified by preparative HPLC (Condition 2, Gradient 2) to afford 4-(1-methyl-1H-1,2,3-triazol-4-yl)-7-(6-((2,2,6,6-tetramethylpiperidin-4-yl)oxy)pyridazin-3-yl)-1H-indazole (4.7 mg, Compound 325) as a solid. LCMS (ES, m/z): 402 [M+H] ¹H NMR (400 MHz, Methanol-d₄) δ 8.71 (s, 1H), 8.62 (s, 1H), 8.38 (d, J=9.4 Hz, 1H), 8.09 (d, J=7.7 Hz, 1H), 7.80 (d, J=7.7 Hz, 1H), 7.26 (d, J=9.4 Hz, 1H), 5.88 (s, 1H), 4.26 (s, 3H), 2.68 (s, 1H), 2.30 (m, 2H), 1.48 (m, 1H), 1.42 (s, 6H), 1.34 (m, 1H), 1.29 (s, 6H).

### Example 81: Synthesis of Compound 326

### Synthesis of Compound 326

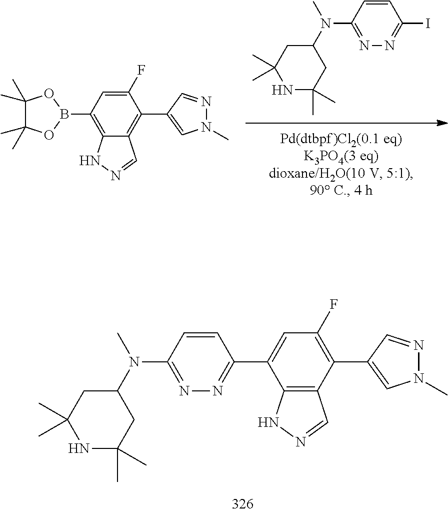

To a stirred solution of 5-fluoro-4-(1-methylpyrazol-4-yl)-7-(4,4,5,5-tetramethyl-1,3,2-dioxaborolan-2-yl)-1H-indazole (100 mg, 0.292 mmol, 1.00 eq.) and Pd(dppf)Cl₂ (21.38 mg, 0.029 mmol, 0.1 eq.) in dioxane (2 mL, 23.608 mmol, 80.78 eq.) were added 6-iodo-N-methyl-N-(2,2,6,6-tetramethylpiperidin-4-yl)pyridazin-3-amine (131.25 mg, 0.350 mmol, 1.2 eq.) and tripotassium phosphate (61.98 mg, 0.876 mmol, 3 eq.) dropwise for 4 hr at 100° C. under a nitrogen atmosphere. The mixture was allowed to cool down to 25° C. The resulting mixture was extracted with ethyl acetate (2×10 mL). The combined organic layers were washed with water (3×10 mL), dried over anhydrous sodium sulfate. After filtration, the filtrate was concentrated under reduced pressure. The crude product was purified by preparative HPLC (Condition 4, Gradient 10). This resulted in 6-[5-fluoro-4-(1-methylpyrazol-4-yl)-1H-indazol-7-yl]-N-methyl-N-(2,2,6,6-tetramethylpiperidin-4-yl)pyridazin-3-amine (18.4 mg, Compound 326) as a solid. LCMS (ES, m/z): 463 [M+H] ¹H NMR (400 MHz, DMSO-d₆) δ 13.19 (s, 1H), 8.49 (s, 1H), 8.43 (s, 1H), 8.21 (d, J=9.8 Hz, 1H), 8.08 (d, J=2.0 Hz, 1H), 7.93 (d, J=12.6 Hz, 1H), 7.26 (d, J=9.8 Hz, 1H), 5.11 (s, 1H), 3.98 (s, 3H), 3.01 (s, 3H), 1.56 (dd, J=12.0, 3.7 Hz, 2H), 1.47 (t, J=12.0 Hz, 2H), 1.29 (s, 6H), 1.11 (s, 6H). ¹⁹F NMR (376 MHz, DMSO-d₆) δ-125.04.

### Example 82: Synthesis of Compound 331

### Synthesis of Intermediate C26

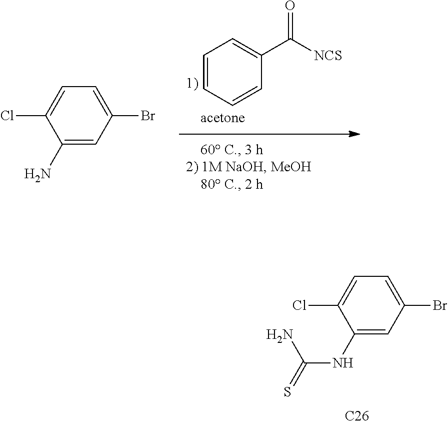

Benzoyl isothiocyanate (3.95 g, 24.21 mmol, 1 equiv) was added dropwise to a stirred solution of 5-bromo-2-chloroaniline (5 g, 24.21 mmol, 1.0 equiv) in acetone (50.0 mL). The reaction mixture was heated to reflux for 3 h. Then poured into ice water, and stirred for an additional 30 min. The precipitate was collected by filtration and washed with more water. This crude material was dissolved in MeOH (50.0 mL) and treated with 1N NaOH (10.0 mL). The reaction mixture was heated to 80° C. for 2 h. After cooling, the reaction mixture was poured into ice water and sufficient aqueous 1N HCl was added to produce a neutra solution. The precipitates from the neutral solution and was collected by filtration and dried to afford 5-bromo-2-chlorophenylthiourea (5 g, C26) as a solid that was taken to the next step without further purification. LCMS (ES, m/z): 265 [M+H]⁺

### Synthesis of Intermediate C27

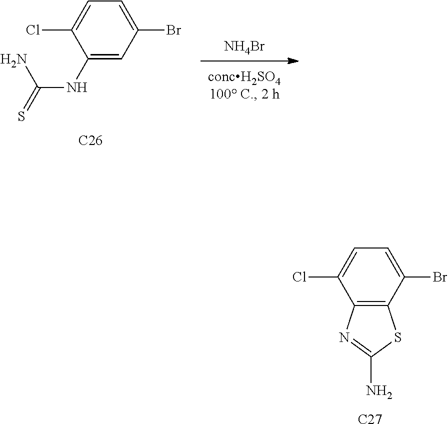 [SMILES: NC(=S)Nc1cc(Br)ccc1Cl ; "NH4Br" ; "C26" ; Nc1nc2c(Cl)ccc(Br)c2s1 ; "C27"]

To a stirred solution of 5-bromo-2-chlorophenylthiourea (5 g, 18.83 mmol, 1.0 equiv) in concentrated H₂SO₄ (15.00 mL) was added amine hydrobromide (1.84 g, 18.82 mmol, 1 equiv) over 1 h, and then the reaction mixture was heated at 100° C. for 2 h. The reaction mixture was cooled to room temperature and then poured into ice water (150 ml), at which point a white precipitate was observed. The acidic was taken to pH-7 using aqueous ammonium hydroxide solution. The solid was collected by filtration, washed with water and dried in vacuum to afford 7-bromo-4-chloro-1,3-benzothiazol-2-amine (4 g, C27) as a solid that was taken to the next step without further purification. LCMS (ES, m/z): 265 [M+H]⁺

### Synthesis of Intermediate C28

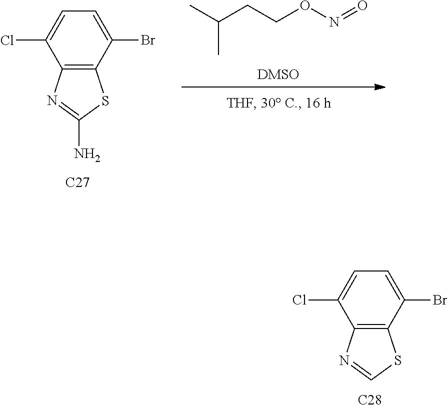

A solution of 7-bromo-4-chloro-1,3-benzothiazol-2-amine (4 g, 15.17 mmol, 1.0 equiv) in THF (30 mL) was added dropwise over 20 min to a solution of isopentyl nitrite (2.35 g, 22.77 mmol, 1.5 equiv) and DMSO (0.12 g, 1.52 mmol, 0.1 equiv) in THF (10.00 mL) at 30° C. The mixture was stirred at 30° C. for 16 h. The mixture was diluted with EtOAc (10 mL) and water (10 mL), the aqueous layer was extracted with EtOAc (2×10 mL). The organic layers were combined and washed with saturated brine (10 mL), dried over anhydrous sodium sulfate and concentrated to give a residue. The residue was purified by flash column chromatography (80 g silica gel column, 20% EA in PE) to afford 7-bromo-4-chloro-1,3-benzothiazole (2 g, C28) as a solid that was taken to the next step without further purification. LCMS (ES, m/z): 247 [M+H]⁺

### Synthesis of Intermediate C29

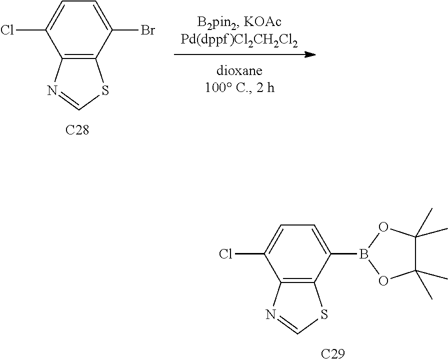 [SMILES: Clc1ccc(Br)c2scnc12 ; "B2pin2, KOAcPd(dppf)Cl2CH2Cl2" ; "C28" ; CC1(C)OB(c2ccc(Cl)c3ncsc23)OC1(C)C ; "C29"]

To a solution of 7-bromo-4-chloro-1,3-benzothiazole (2 g, 8.04 mmol, 1.0 equiv) and 4,4,5,5-tetramethyl-2-(tetramethyl-1,3,2-dioxaborolan-2-yl)-1,3,2-dioxaborolane (3.07 g, 12.07 mmol, 1.5 equiv) in 1,4-dioxane (20 mL) were added KOAc (2.37 g, 24.144 mmol, 3 equiv) and Pd(dppf)Cl₂·CH₂Cl₂ (0.66 g, 0.80 mmol, 0.1 equiv). After stirring for 2 hat 100° C. under a nitrogen atmosphere, the resulting mixture was filtered and the filtrated was concentrated under reduced pressure to afford 4-chloro-7-(4,4,5,5-tetramethyl-1,3,2-dioxaborolan-2-yl)-1,3-benzothiazole (2 g, C29) as an oil that was taken to the next step without further purification. LCMS (ES, m/z): 295 [M+H]⁺

### Synthesis of Intermediate C30

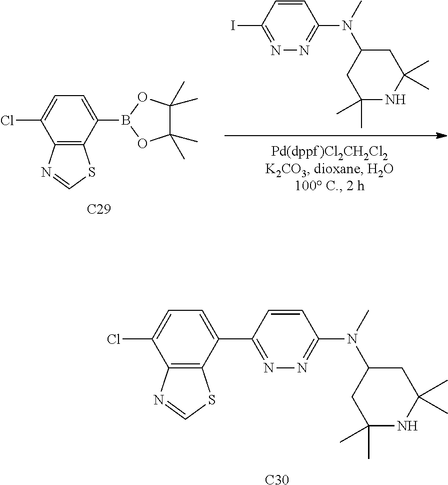 [SMILES: CC1(C)OB(c2ccc(Cl)c3ncsc23)OC1(C)C ; CN(c1ccc(I)nn1)C1CC(C)(C)NC(C)(C)C1 ; "C29" ; CN(c1ccc(-c2ccc(Cl)c3ncsc23)nn1)C1CC(C)(C)NC(C)(C)C1 ; "C30"]

To a solution of 4-chloro-7-(4,4,5,5-tetramethyl-1,3,2-dioxaborolan-2-yl)-1,3-benzothiazole (2 g, 6.76 mmol, 1.0 equiv) and 6-iodo-N-methyl-N-(2,2,6,6-tetramethylpiperidin-4-yl)pyridazin-3-amine (2.53 g, 6.76 mmol, 1 equiv) in 1,4-dioxane (20 mL, 227.0 mmol, 33.55 equiv) and H₂O (5 mL, 277.54 mmol, 41.02 equiv) were added K₂CO₃ (2.81 g, 20.298 mmol, 3 equiv) and Pd(dppf)Cl₂·CH₂Cl₂ (0.55 g, 0.67 mmol, 0.1 equiv). After stirring for 2 hr at 100° C. under a nitrogen atmosphere, the resulting mixture was concentrated under reduced pressure. The residue was purified by Prep-TLC/silica gel column chromatography, eluted with CH₂Cl₂/MeOH (9/1) to afford 6-(4-chloro-1,3-benzothiazol-7-yl)-N-methyl-N-(2,2,6,6-tetramethylpiperidin-4-yl)pyridazin-3-amine) (1.8 g, C30) as a solid that was taken to the next step without further purification. LCMS (ES, m/z): 415 [M+H]⁺

### Synthesis of Compound 331

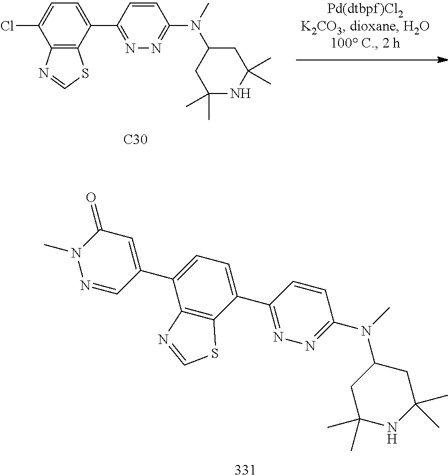

To a solution of 6-(4-chloro-1,3-benzothiazol-7-yl)-N-methyl-N-(2,2,6,6-tetramethylpiperidin-4-yl) pyridazin-3-amine (80 mg, 0.19 mmol, 1.0 equiv) and 1-methyl-6-oxopyridazin-4-ylboronic acid (59.21 mg, 0.38 mmol, 2 equiv) in 1,4-dioxane (2 mL, 22.70 mmol, 118.04 equiv) and H₂O (0.5 mL, 27.75 mmol, 144.31 equiv) were added K₂CO₃ (79.74 mg, 0.56 mmol, 3 equiv) and H₂O (0.5 mL, 27.75 mmol, 144.31 equiv). After stirring for 2 h at 100° C. under a nitrogen atmosphere, the resulting mixture was concentrated under reduced pressure. The residue was purified by Prep-TLC/silica gel column chromatography, eluted with CH₂Cl₂/MeOH (9/1) to afford crude product. The crude product was purified by prep-PLC (Condition 7, Gradient 10) to afford 2-methyl-5-(7-{6-[methyl(2,2,6,6-tetramethylpiperidin-4-yl)amino]pyridazin-3-yl}-1,3-benzothiazol-4-yl) pyridazin-3-one (12.5 mg, Compound 331) as a solid.

Compounds 330, 332, 333, 340-342, and 405-407 were synthesized using the general synthetic scheme of Compound 331. Corresponding data for Compounds 330-333, 340-342, and 405-408 are shown in Table 6.

TABLE 6
Exemplary compounds

| Com- | |
|---|---|
| pound | Analysis data |
| 330 | LCMS (ESI, m/z): 489 [M + H]. |
| | ¹H-NMR (400 MHz, DMSO-d₆) δ 9.60 (s, 1H), 9.52 (d, J = 1.8 |
| | Hz, 1H), 8.35 (d, J = 9.9 Hz, 1H), 8.29 (d, J = 8.1 Hz, 1H), 8.06 |
| | (d, J = 7.9 Hz, 1H), 7.81 (d, J = 1.8 Hz, 1H), 7.30 (d, J = 9.8 |
| | Hz, 1H), 5.05 (s, 1H), 4.13 (s, 3H), 3.03 (s, 3H), 1.56 (dd, J = |
| | 12.1, 3.7 Hz, 2H), 1.49 (d, J = 12.1 Hz, 2H), 1.29 (s, 6H), 1.11 |
| | (s, 6H). |
| 331 | LCMS (ESI, m/z): 490 [M + H]. |
| | ¹H-NMR (400 MHz, DMSO-d₆) δ 9.60 (s, 1H), 8.53 (d, J = |
| | 2.2 Hz, 1H), 8.34 (d, J = 9.8 Hz, 1H), 8.26 (d, J = 8.1 Hz, |
| | 1H), 7.99 (d, J = 7.9 Hz, 1H), 7.52 (d, J = 2.2 Hz, 1H), 7.29 |
| | (d, J = 9.8 Hz, 1H), 5.05 (s, 1H), 3.75 (s, 3H), 3.02 (s, 3H), |
| | 1.56 (dd, J = 12.0, 3.6 Hz, 2H), 1.47 (t, J = 12.0 Hz, 2H), 1.29 |
| | (s, 6H), 1.11 (s, 6H). |
| 332 | LCMS (ESI, m/z): 489 [M + H]. |
| | ¹H-NMR (400 MHz, DMSO-d₆) δ 9.65 (s, 1H), 8.63 (s, 1H), |
| | 8.51 (d, J = 8.1 Hz, 1H), 8.34-8.24 (m, 2H), 8.08 (d, J = 0.9 |
| | Hz, 1H), 7.30 (d, J = 9.8 Hz, 1H), 5.06 (s, 1H), 3.49 (s, 3H), |
| | 3.03 (s, 3H), 1.61-1.52 (m, 2H), 1.47 (t, J = 12.0 Hz, 2H), |
| | 1.29 (s, 6H), 1.11 (s, 6H). |
| 333 | LCMS (ESI, m/z): 489 [M + H]. |
| | ¹H-NMR (400 MHz, DMSO-d₆) δ 9.54 (s, 1H), 8.31 (d, J = 9.8 |
| | Hz, 1H), 8.21 (d, J = 8.0 Hz, 1H), 7.85 (d, J = 7.9 Hz, 1H), 7.80 |
| | (d, J = 7.1 Hz, 1H), 7.29 (d, J = 9.8 Hz, 1H), 6.98 (d, J = 1.9 |
| | Hz, 1H), 6.80 (dd, J = 7.0, 2.1 Hz, 1H), 5.04 (s, 1H), 3.51 (s, |
| | 3H), 3.02 (s, 3H), 1.56 (dd, J = 12.0, 3.6 Hz, 2H), 1.47 (t, J = |
| | 12.0 Hz, 2H), 1.29 (s, 6H), 1.11 (s, 6H). |
| 340 | LCMS (ESI, m/z): 462 [M + H]. |
| | ¹H NMR (400 MHz, DMSO-d₆) δ 9.51 (s, 1H), 8.66 (s, 1H), |
| | 8.32 (s, 1H), 8.26 (d, J = 9.8 Hz, 1H), 8.10 (d, J = 8.1 Hz, 1H), |
| | 7.95 (d, J = 7.9 Hz, 1H), 7.27 (d, J = 9.7 Hz, 1H), 5.01 (s, 1H), |
| | 3.96 (s, 3H), 3.01 (s, 3H), 1.56 (dd, J = 12.3, 3.6 Hz, 2H), 1.47 |
| | (t, J = 11.9 Hz, 2H), 1.29 (s, 6H), 1.11 (s, 6H). |
| 341 | LCMS (ESI, m/z): 462 [M + H]. |
| | ¹H-NMR (400 MHz, DMSO-d₆) δ 12.76 (s, 1H), 9.44 (s, 1H), |
| | 8.25 (d, J = 9.8 Hz, 1H), 8.13 (d, J = 8.0 Hz, 1H), 7.96 (s, 1H), |
| | 7.70-7.60 (m, 1H), 7.28 (d, J = 9.8 Hz, 1H), 5.02 (s, 1H), 3.01 |
| | (s, 3H), 2.41 (d, J = 12.8 Hz, 3H), 1.56 (d, J = 9.8 Hz, 2H), 1.47 |
| | (t, J = 12.0 Hz, 2H), 1.29 (s, 6H), 1.11 (s, 6H). |
| 342 | LCMS (ESI, m/z): 463 [M + H]. |
| | ¹H-NMR (400 MHz, DMSO-d₆) δ 9.61 (s, 1H), 8.29 (d, J = 9.8 |
| | Hz, 1H), 8.22 (d, J = 8.1 Hz, 1H), 8.05 (s, 1H), 7.97 (d, J = 8.0 |
| | Hz, 1H), 7.29 (d, J = 9.8 Hz, 1H), 5.05 (s, 1H), 3.02 (s, 3H), |
| | 2.58 (s, 3H), 1.57 (dd, J = 12.1, 3.7 Hz, 2H), 1.49 (t, J = 12.2 |
| | Hz, 2H), 1.30 (s, 6H), 1.13 (s, 6H). |
| 405 | LCMS (ESI, m/z): 489 [M + H]. |
| | ¹H NMR (400 MHz, DMSO-d₆) δ 9.55 (s, 1H), 8.36-8.27 (m, |
| | 2H), 8.24 (d, J = 8.0 Hz, 1H), 7.91 (d, J = 7.9 Hz, 1H), 7.56 (d, |
| | J = 5.5 Hz, 1H), 7.41 (s, 1H), 7.30 (d, J = 9.9 Hz, 1H), 5.05 (s, |
| | 1H), 3.94 (s, 3H), 3.02 (s, 3H), 1.56 (dd, J = 11.9, 3.6 Hz, 2H), |
| | 1.47 (t, J = 12.0 Hz, 2H), 1.29 (s, 6H), 1.11 (s, 6H). |
| 406 | LCMS (ESI, m/z): 489 [M + H]. |
| | ¹H-NMR (400 MHz, DMSO-d₆) δ 11.90 (s, 1H), 9.51 (s, 1H), |
| | 8.28 (d, J = 9.9 Hz, 1H), 8.23 (d, J = 2.7 Hz, 1H), 8.19-8.09 |
| | (m, 2H), 7.79 (d, J = 8.0 Hz, 1H), 7.28 (d, J = 9.8 Hz, 1H), 6.50 |
| | (d, J = 9.6 Hz, 1H), 5.03 (s, 1H), 3.01 (s, 3H), 1.56 (dd, J = |
| | 12.1, 3.6 Hz, 2H), 1.47 (t, J = 12.0 Hz, 2H), 1.29 (s, 6H), 1.11 |
| | (s, 6H). |
| 407 | LCMS (ESI, m/z): 489 [M + H]. |
| | ¹H-NMR (400 MHz, DMSO-d₆) δ 9.52 (s, 1H), 8.44 (d, J = 2.6 |
| | Hz, 1H), 8.30 (d, J = 9.8 Hz, 1H), 8.18 (d, J = 8.0 Hz, 1H), 8.12 |
| | (dd, J = 9.4, 2.7 Hz, 1H), 7.77 (d, J = 7.9 Hz, 1H), 7.28 (d, J = |
| | 9.8 Hz, 1H), 6.55 (d, J = 9.5 Hz, 1H), 5.03 (s, 1H), 3.57 (s, 3H), |
| | 3.01 (s, 3H), 1.55 (dd, J = 12.0, 3.6 Hz, 2H), 1.46 (t, J = 12.0 |
| | Hz, 2H), 1.29 (s, 6H), 1.11 (s, 6H). |

### Example 83: Synthesis of Compound 335

### Synthesis of Compound 335

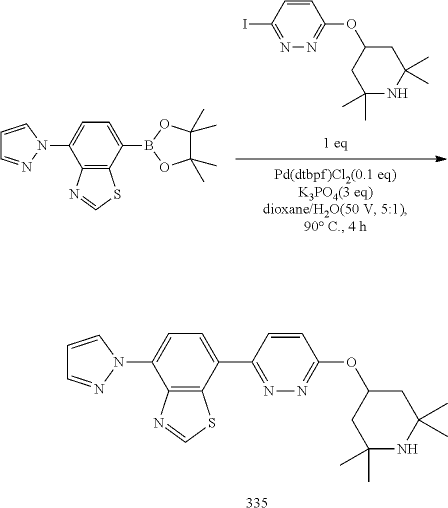 [SMILES: CC1(C)OB(c2ccc(-n3cccn3)c3ncsc23)OC1(C)C ; CC1(C)CC(Oc2ccc(I)nn2)CC(C)(C)N1 ; CC1(C)CC(Oc2ccc(-c3ccc(-n4cccn4)c4ncsc34)nn2)CC(C)(C)N1 ; "335"]

A solution of 4-(pyrazol-1-yl)-7-(4,4,5,5-tetramethyl-1,3,2-dioxaborolan-2-yl)-1,3-benzothiazole (70 mg, 0.210 mmol, 1.00 eq.), 3-iodo-6-[(2,2,6,6-tetramethylpiperidin-4-yl)oxy]pyridazine (75.73 mg, 0.210 mmol, 1 eq.), Pd(dtbpf)Cl₂ (13.66 mg, 0.021 mmol, 0.1 eq.) and tripotassium phosphate (133.51 mg, 0.630 mmol, 3 eq.) in 1,4-dioxane (3.5 mL) and water (0.7 mL) was stirred for 4 hr at 90° C. under a nitrogen atmosphere. The mixture was allowed to cool down to room temperature. The resulting mixture was diluted with water (30 mL). The resulting mixture was extracted with ethyl acetate (3×30 mL), dried over anhydrous sodium sulfate. After filtration, the filtrate was concentrated under reduced pressure. The residue was purified by silica gel column chromatography, eluted with petroleum ether/ethyl acetate (1:1). The crude product was purified by preparative HPLC (Condition 4, Gradient 14) to afford 4-(pyrazol-1-yl)-7-{6-[(2,2,6,6-tetramethylpiperidin-4-yl)oxy]pyridazin-3-yl}-1,3-benzothiazole (22.1 mg, 24.23%) as a solid. LCMS (ES, m/z): 435 [M+H] ¹H NMR (400 MHz, DMSO-d₆) δ 9.65 (s, 1H), 9.23 (d, J=2.6 Hz, 1H), 8.56 (d, J=9.4 Hz, 1H), 8.41 (d, J=8.4 Hz, 1H), 8.26 (d, J=8.3 Hz, 1H), 7.91 (d, J=1.7 Hz, 1H), 7.39 (d, J=9.3 Hz, 1H), 6.67 (t, J=2.2 Hz, 1H), 5.79 (tt, J=11.2, 4.2 Hz, 1H), 2.51-2.45 (m, 2H), 2.13 (dd, J=11.9, 4.1 Hz, 2H), 1.28 (s, 6H), 1.13 (s, 6H).

### Example 84: Synthesis of Compound 337

### Synthesis of Intermediate C31

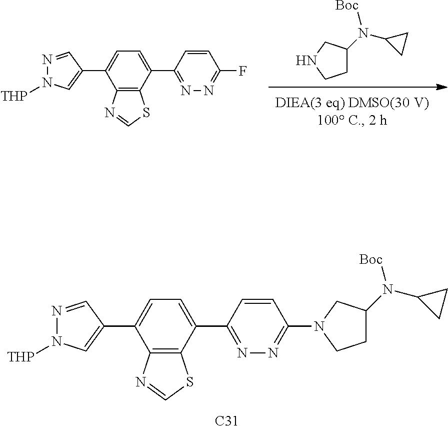

A mixture of 7-(6-fluoropyridazin-3-yl)-4-[1-(oxan-2-yl) pyrazol-4-yl]-1,3-benzothiazole (100.00 mg, 0.2 mmol, 1.00 eq.), tert-butyl N-cyclopropyl-N-(pyrrolidin-3-yl) carbamate (118.67 mg, 0.5 mmol, 2.00 eq.) and N,N′-diisopropylethylamine (101.50 mg, 0.7 mmol, 3.00 eq.) in dimethylsulfoxide (5.0 mL) was stirred for 2 hr at 100° C. The mixture was allowed to cool down to room temperature. The resulting mixture was diluted with water (20.0 mL). The precipitated solids were collected by filtration and washed with water (2×20.0 mL). The resulting mixture was concentrated under vacuum to afford tert-butyl N-cyclopropyl-N-[1-(6-{4-[1-(oxan-2-yl) pyrazol-4-yl]-1,3-benzothiazol-7-yl} pyridazin-3-yl) pyrrolidin-3-yl] carbamate (230 mg, C31) as a solid that was taken to the next step without further purification. LCMS (ES, m/z): 588 [M+H]⁺

### Synthesis of Compound 337

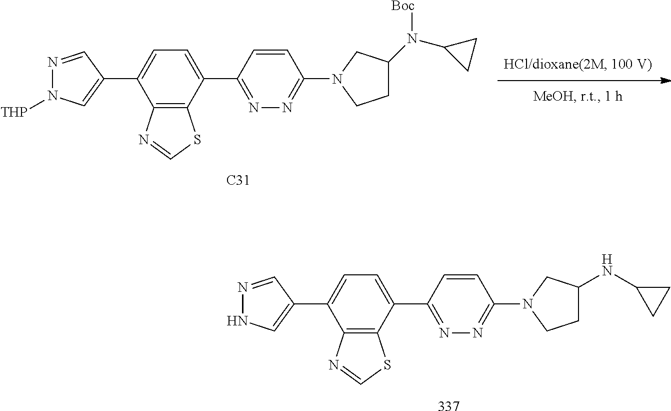

A solution of N-tert-butyl-1-(6-{4-[1-(oxan-2-yl) pyrazol-4-yl]-1,3-benzothiazol-7-yl}pyridazin-3-yl) pyrrolidin-3-amine (120.00 mg, 0.2 mmol, 1.00 eq.) in hydrochloric acid (gas) in 1,4-dioxane (1.2 mL) and methanol (3.6 mL) was stirred for 1 hr at room temperature. The resulting mixture was concentrated under vacuum. The crude product was purified by preparative HPLC (Condition 7, Gradient 3) to afford N-tert-butyl-1-{6-[4-(1H-pyrazol-4-yl)-1,3-benzothiazol-7-yl] pyridazin-3-yl} pyrrolidin-3-amine (26 mg, 26%) as a solid. LCMS (ES, m/z): 404 [M+H]+¹H NMR (400 MHz, DMSO-d₆) δ 13.08 (s, 1H), 9.51 (s, 1H), 8.68 (s, 1H), 8.40 (s, 1H), 8.27 (d, J=9.7 Hz, 1H), 8.11 (d, J=8.1 Hz, 1H), 7.98 (d, J=8.0 Hz, 1H), 7.05 (d, J=9.6 Hz, 1H), 3.73 (d, J=8.8 Hz, 1H), 3.65 (d, J=8.1 Hz, 1H), 3.54 (d, J=5.4 Hz, 2H), 3.5-3.4 (m, 1H), 2.15 (dd, J=7.5, 4.5 Hz, 2H), 1.95 (dd, J=12.3, 6.3 Hz, 1H), 0.43 (dd, J=6.5, 1.7 Hz, 2H), 0.31-0.23 (m, 2H).

### Example 85: Synthesis of Compound 345

### Synthesis of Intermediate C32

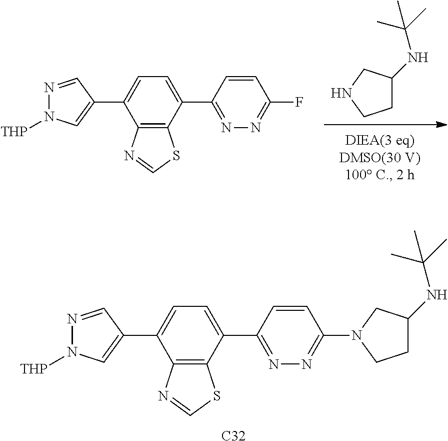

A mixture of 7-(6-fluoropyridazin-3-yl)-4-[1-(oxan-2-yl) pyrazol-4-yl]-1,3-benzothiazole (100.00 mg, 0.2 mmol, 1.00 eq.), N-tert-butylpyrrolidin-3-amine (41.02 mg, 0.2 mmol, 1.10 eq.) and N,N′-diisopropylethylamine (101.65 mg, 0.7 mmol, 3.00 eq.) in dimethylsulfoxide (5.0 mL) was stirred for 2 hr at 100° C. The mixture was allowed to cool down to room temperature. The resulting mixture was diluted with water (20.0 mL). The precipitated solids were collected by filtration and washed with water (2×20.0 mL). The resulting mixture was concentrated under vacuum to afford N-tert-butyl-1-(6-{4-[1-(oxan-2-yl) pyrazol-4-yl]-1,3-benzothiazol-7-yl}pyridazin-3-yl) pyrrolidin-3-amine (120 mg, C32) as a solid that was taken to the next step without further purification. LCMS (ES, m/z): 504 [M+H]⁺

### Synthesis of Compound 345

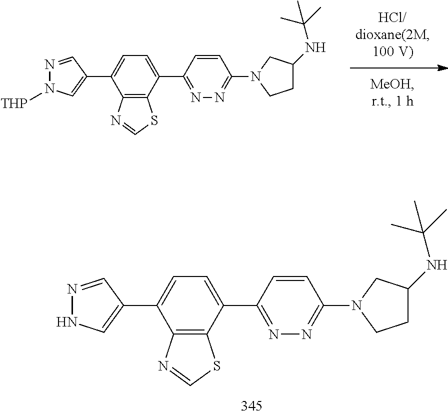

A solution of N-tert-butyl-1-(6-{4-[1-(oxan-2-yl) pyrazol-4-yl]-1,3-benzothiazol-7-yl}pyridazin-3-yl) pyrrolidin-3-amine (120.00 mg, 0.2 mmol, 1.00 eq.) in hydrochloric acid (gas) in 1,4-dioxane (1.2 mL) and methanol (3.6 mL) was stirred for 1 hr at room temperature. The resulting mixture was concentrated under vacuum. The crude product was purified by preparative HPLC (Condition 7, Gradient 3) to afford N-tert-butyl-1-{6-[4-(1H-pyrazol-4-yl)-1,3-benzothiazol-7-yl] pyridazin-3-yl} pyrrolidin-3-amine (26.20 mg, Compound 345) as a solid. LCMS (ES, m/z): 420 [M+H] ¹H NMR (400 MHz, DMSO-d₆) δ 13.09 (s, 1H), 9.51 (s, 1H), 8.68 (s, 1H), 8.40 (s, 1H), 8.26 (d, J=9.7 Hz, 1H), 8.11 (d, J=8.1 Hz, 1H), 7.98 (d, J=7.9 Hz, 1H), 7.04 (d, J=9.6 Hz, 1H), 3.84 (s, 1H), 3.70 (s, 1H), 3.52 (dt, J=26.9, 8.1 Hz, 2H), 3.13 (dd, J=10.2, 7.2 Hz, 1H), 2.20 (d, J=6.2 Hz, 1H), 1.79 (dt, J=11.9, 8.5 Hz, 1H), 1.73 (s, 1H), 1.11 (s, 9H).

### Example 86: Synthesis of Compound 346

### Synthesis of Intermediate C33

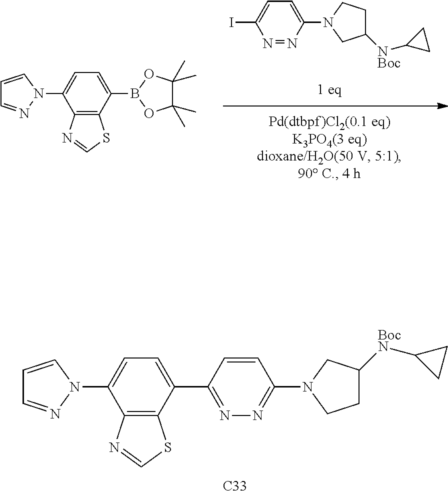 [SMILES: CC(C)(C)OC(=O)N(C1CC1)C1CCN(c2ccc(I)nn2)C1 ; CC1(C)OB(c2ccc(-n3cccn3)c3ncsc23)OC1(C)C ; CC(C)(C)OC(=O)N(C1CC1)C1CCN(c2ccc(-c3ccc(-n4cccn4)c4ncsc34)nn2)C1 ; "C33"]

A solution of 4-(pyrazol-1-yl)-7-(4,4,5,5-tetramethyl-1,3,2-dioxaborolan-2-yl)-1,3-benzothiazole (60 mg, 0.183 mmol, 1.00 eq.), tert-butyl N-cyclopropyl-N-[1-(6-iodopyridazin-3-yl)pyrrolidin-3-yl]carbamate (78.90 mg, 0.183 mmol, 1.0 eq.), Pd(DtBPF)Cl₂ (11.95 mg, 0.018 mmol, 0.1 eq.) and tripotassium phosphate (116.77 mg, 0.549 mmol, 3 eq.) in 1,4-dioxane (2.5 mL) and water (0.5 mL) was stirred for 4 hr at 90° C. under a nitrogen atmosphere. The mixture was allowed to cool down to room temperature. The resulting mixture was diluted with water (30 mL). The resulting mixture was extracted with ethyl acetate (3×30 mL) and dried over anhydrous sodium sulfate. After filtration, the filtrate was concentrated under reduced pressure. The residue was purified by silica gel column chromatography, eluted with petroleum ether/ethyl acetate (1:1) to afford tert-butyl N-cyclopropyl-N-(1-{6-[4-(pyrazol-1-yl)-1,3-benzothiazol-7-yl]pyridazin-3-yl}pyrrolidin-3-yl)carbamate (40 mg, C33) as a solid that was taken to the next step without further purification. LCMS (ES, m/z): 504 [M+H]⁺

### Synthesis of Compound 346

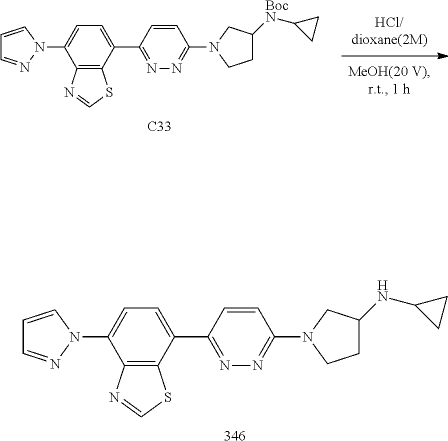 [SMILES: CC(C)(C)OC(=O)N(C1CC1)C1CCN(c2ccc(-c3ccc(-n4cccn4)c4ncsc34)nn2)C1 ; "C33" ; c1cnn(-c2ccc(-c3ccc(N4CCC(NC5CC5)C4)nn3)c3scnc23)c1 ; "346"]

A solution of tert-butyl N-cyclopropyl-N-(1-{6-[4-(pyrazol-1-yl)-1,3-benzothiazol-7-yl]pyridazin-3-yl}pyrrolidin-3-yl)carbamate (40 mg, 0.078 mmol, 1.00 eq.) in hydrochloric acid (gas)in 1,4-dioxane (0.8 mL) and methanol (0.8 mL) was stirred for 1 hr at room temperature. The resulting mixture was concentrated under reduced pressure. The crude product was purified by preparative HPLC (Condition 7, Gradient 8) to afford N-cyclopropyl-1-{6-[4-(pyrazol-1-yl)-1,3-benzothiazol-7-yl]pyridazin-3-yl}pyrrolidin-3-amine (12 mg, Compound 346) as a solid. LCMS (ES, m/z): 504 [M+H] ¹H-NMR (400 MHz, DMSO-d₆) δ 9.62 (s, 1H), 9.18 (d, J=2.5 Hz, 1H), 8.32-8.24 (m, 2H), 8.20 (d, J=8.3 Hz, 1H), 7.89 (d, J=1.7 Hz, 1H), 7.08 (d, J=9.6 Hz, 1H), 6.65 (dd, J=2.5, 1.8 Hz, 1H), 3.75 (dd, J=10.4, 5.7 Hz, 1H), 3.66 (s, 1H), 3.54 (dt, J=11.1, 5.9 Hz, 2H), 3.43-3.42 (m, 1H), 2.15 (qd, J=7.7, 6.7, 4.8 Hz, 2H), 1.95 (dq, J=12.8, 6.6 Hz, 1H), 0.49-0.37 (m, 2H), 0.35-0.20 (m, 2H).

### Example 87: Synthesis of Compound 347

### Synthesis of Intermediate C34

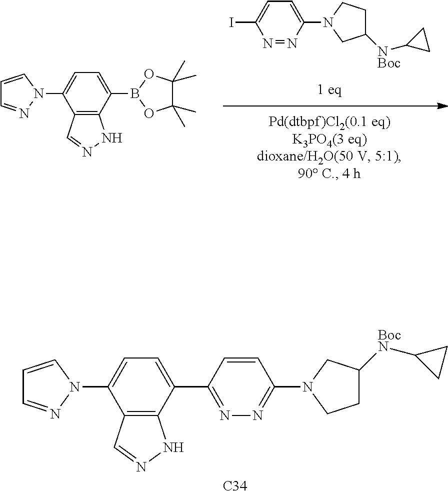 [SMILES: CC(C)(C)OC(=O)N(C1CC1)C1CCN(c2ccc(I)nn2)C1 ; CC1(C)OB(c2ccc(-n3cccn3)c3cn[nH]c23)OC1(C)C ; CC(C)(C)OC(=O)N(C1CC1)C1CCN(c2ccc(-c3ccc(-n4cccn4)c4cn[nH]c34)nn2)C1 ; "C34"]

A solution of 4-(pyrazol-1-yl)-7-(4,4,5,5-tetramethyl-1,3,2-dioxaborolan-2-yl)-1H-indazole (60 mg, 0.193 mmol, 1.00 eq.), tert-butyl N-cyclopropyl-N-[1-(6-iodopyridazin-3-yl)pyrrolidin-3-yl]carbamate (83.24 mg, 0.193 mmol, 1 eq.), Pd(dtbpf)Cl₂ (12.61 mg, 0.019 mmol, 0.1 eq.) and tripotassium phosphate (123.19 mg, 0.579 mmol, 3 eq.) in 1,4-dioxane (2.5 mL) and water (0.5 mL) was stirred for 4 hr at 90° C. under a nitrogen atmosphere. The mixture was allowed to cool down to room temperature. The resulting mixture was diluted with water (30 mL). The resulting mixture was extracted with ethyl acetate (3×30 mL) and dried over anhydrous sodium sulfate. After filtration, the filtrate was concentrated under reduced pressure. The residue was purified by silica gel column chromatography, eluted with petroleum ether/ethyl acetate (1:1) to afford tert-butyl N-cyclopropyl-N-(1-{6-[4-(pyrazol-1-yl)-1H-indazol-7-yl]pyridazin-3-yl}pyrrolidin-3-yl)carbamate (20 mg, C34) as a solid that was taken to the next step without further purification. LCMS (ES, m/z): 487 [M+H]⁺

### Synthesis of Compound 347

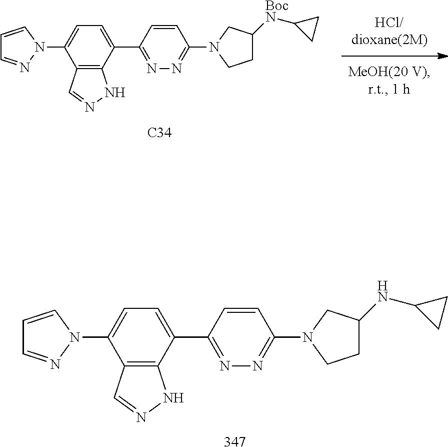 [SMILES: CC(C)(C)OC(=O)N(C1CC1)C1CCN(c2ccc(-c3ccc(-n4cccn4)c4cn[nH]c34)nn2)C1 ; "C34" ; c1cnn(-c2ccc(-c3ccc(N4CCC(NC5CC5)C4)nn3)c3[nH]ncc23)c1 ; "347"]

A solution of tert-butyl N-cyclopropyl-N-(1-{6-[4-(pyrazol-1-yl)-1H-indazol-7-yl]pyridazin-3-yl}pyrrolidin-3-yl)carbamate (20 mg, 0.040 mmol, 1.00 eq.) in hydrochloric acid (gas)in 1,4-dioxane (0.4 mL) and methanol (0.4 mL) was stirred for 1 hr at room temperature. The resulting mixture was concentrated under reduced pressure. The crude product was purified by preparative HPLC (Condition 4, Gradient 15) to afford N-cyclopropyl-1-{6-[4-(pyrazol-1-yl)-1H-indazol-7-yl]pyridazin-3-yl}pyrrolidin-3-amine (3.9 mg, Compound 347) as a solid. LCMS (ES, m/z): 387 [M+H]+H-NMR (400 MHz, DMSO-d₆) δ 13.30 (s, 1H), 8.70 (d, J=2.5 Hz, 1H), 8.63 (d, J=1.7 Hz, 1H), 8.21 (d, J=9.6 Hz, 1H), 8.00 (d, J=8.1 Hz, 1H), 7.92 (d, J=1.7 Hz, 1H), 7.61 (d, J=8.0 Hz, 1H), 7.06 (dd, J=9.6, 1.3 Hz, 1H), 6.66 (t, J=2.2 Hz, 1H), 3.76 (dd, J=10.6, 5.9 Hz, 1H), 3.65 (t, J=7.4 Hz, 1H), 3.54 (td, J=9.7, 7.7, 4.1 Hz, 2H), 3.44 (d, J=4.5 Hz, 1H), 2.14 (dt, J=6.6, 3.3 Hz, 2H), 2.02-1.89 (m, 1H), 0.49-0.37 (m, 2H), 0.33-0.20 (m, 2H).

### Example 88: Synthesis of Compound 348

### Synthesis of Intermediate C35

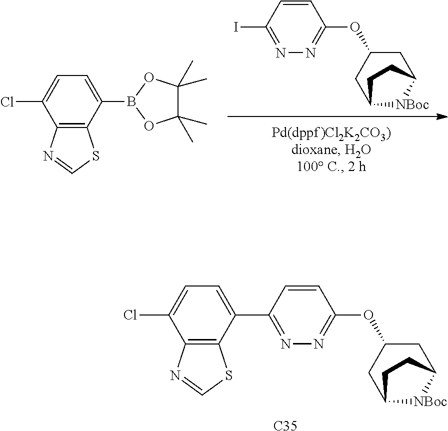

To a solution of 4-chloro-7-(4,4,5,5-tetramethyl-1,3,2-dioxaborolan-2-yl)-1,3-benzothiazole (2 g, 6.76 mmol, 1.0 equiv) and tert-butyl (exo)-3-[(6-iodopyridazin-3-yl)oxy]-8-azabicyclo[3.2.1]octane-8-carboxylate (2.92 g, 6.76 mmol, 1 equiv) in 1,4-dioxane (20 mL) and H₂O (5 mL) were added K₂CO₃ (2.81 g, 20.298 mmol, 3 equiv) and Pd(dppf)Cl₂·CH₂Cl₂ (0.55 g, 0.67 mmol, 0.1 equiv). After stirring for 2 h at 100° C. under a nitrogen atmosphere, the resulting mixture was concentrated under reduced pressure. The residue was purified by silica gel column chromatography, eluted with PE/EA (1/1) to afford tert-butyl (exo)-3-{[6-(4-chloro-1,3-benzothiazol-7-yl) pyridazin-3-yl]oxy}-8-azabicyclo[3.2.1]octane-8-carboxylate (1.5 g, C35) as a solid that was taken to the next step without further purification. LCMS (ES, m/z): 473 [M+H]

### Synthesis of Intermediate C36

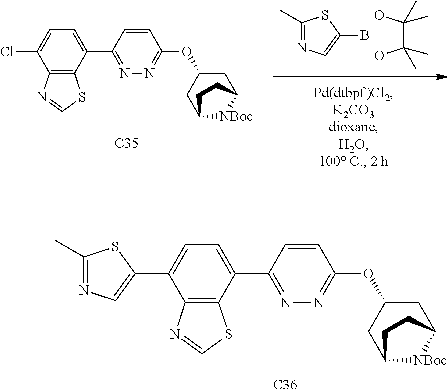

To a solution of tert-butyl (1R,3R,5S)-3-{[6-(4-chloro-1,3-benzothiazol-7-yl)pyridazin-3-yl]oxy}-8-azabicyclo[3.2.1]octane-8-carboxylate (100 mg, 0.21 mmol, 1.0 equiv) and 2-methyl-5-(4,4,5,5-tetramethyl-1,3,2-dioxaborolan-2-yl)-1,3-thiazole (95.19 mg, 0.42 mmol, 2 equiv) in 1,4-dioxane (2 mL) and H₂O (0.5 mL) were added K₂CO₃ (87.66 mg, 0.63 mmol, 3 equiv) and Pd(DtBPF)Cl₂ (13.78 mg, 0.021 mmol, 0.1 equiv). After stirring for 2 hr at 100° C. under a nitrogen atmosphere, the resulting mixture was concentrated under reduced pressure. The residue was purified by Prep-TLC/silica gel column chromatography, eluted with PE/EA (7:3) to afford tert-butyl (1R,3R,5S)-3-({6-[4-(2-methyl-1,3-thiazol-5-yl)-1,3-benzothiazol-7-yl] pyridazin-3-yl}oxy)-8-azabicyclo[3.2.1]octane-8-carboxylate (80 mg, C36) as a solid that was taken to the next step without further purification. LCMS (ES, m/z): 536 [M+H]⁺

### Synthesis of Compound 348

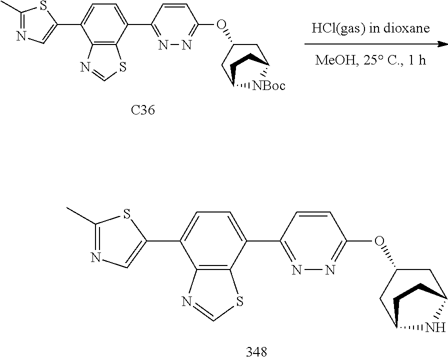

To a solution of tert-butyl (exo)-3-({6-[4-(2-methyl-1,3-thiazol-5-yl)-1,3-benzothiazol-7-yl]pyridazin-3-yl}oxy)-8-azabicyclo[3.2.1]octane-8-carboxylate (80 mg) in HCl(gas)in 1,4-dioxane (1 mL) and MeOH (1 mL). After stirring for 1h at 25° C. under a nitrogen atmosphere, the resulting mixture was concentrated under reduced pressure. The crude product was purified by preparative HPLC (Condition 7, Gradient 8) to afford 7-{6-[(exo)-8-azabicyclo[3.2.1]octan-3-yloxy]pyridazin-3-yl}-4-(2-methyl-1,3-thiazol-5-yl)-1,3-benzothiazole (4.7 mg, Compound 348) as a solid. Compounds 349, 362, 363, 368, 403, and 408 were synthesized using the general synthetic scheme of Compound 348. Corresponding data for Compounds 348, 349, 362, 363, 403, and 408 are shown in Table 7 below.

TABLE 7
Exemplary compounds

| Com- | |
|---|---|
| pound | Analysis data |
| 348 | LCMS (ESI, m/z): 436 [M + H]. |
| | ¹H-NMR (400 MHz, DMSO-d₆) δ 9.62 (s, 1H), 8.61 (s, |
| | 1H), 8.56 (d, J = 9.5 Hz, 1H), 8.29 (d, J = 8.2 Hz, 1H), |
| | 8.18 (d, J = 8.0 Hz, 1H), 7.36 (d, J = 9.4 Hz, 1H), 5.62 (tt, |
| | J = 10.9, 6.0 Hz, 1H), 3.53 (s, 2H), 2.74 (s, 3H), 2.25 - |
| | 2.15 (m, 2H), 1.76 (d, J = 2.0 Hz, 4H), 1.63 (td, J = 11.5, |
| | 2.9 Hz, 2H). |
| 349 | LCMS (ESI, m/z): 430 [M + H]. |
| | ¹H-NMR (400 MHz, DMSO-d₆) δ 9.60 (s, 1H), 8.62- |
| | 8.55 (m, 2H), 8.37 (d, J = 8.0 Hz, 1H), 7.93 (d, J = 7.9 Hz, |
| | 1H), 7.82 (d, J = 1.6 Hz, 1H), 7.75 (dd, J = 5.2, 1.7 Hz, |
| | 1H), 7.38 (d, J = 9.4 Hz, 1H), 5.63 (tt, J = 11.0, 6.0 Hz, |
| | 1H), 3.55 (s, 2H), 2.59 (s, 3H), 2.21 (ddd, J = 12.2, 6.1, |
| | 2.9 Hz, 2H), 1.77 (d, J = 2.5 Hz, 4H), 1.64 (td, J = 11.5, |
| | 3.0 Hz, 2H). |
| 362 | LCMS (ESI, m/z): 447 [M + H]. |
| | ¹H NMR (400 MHz, DMSO-d₆) δ 9.65 (s, 1H), 9.57-9.49 |
| | (m, 1H), 8.64-8.53 (m, 1H), 8.41 (d, J = 8.0 Hz, 1H), |
| | 8.10 (d, J = 7.9 Hz, 1H), 7.87-7.78 (m, 1H), 7.43-7.29 |
| | (m, 1H), 5.63 (tq, J = 11.0, 5.5, 5.1 Hz, 1H), 4.13 (s, 3H), |
| | 3.53 (s, 2H), 2.21 (ddd, J = 12.0, 6.0, 3.1 Hz, 2H), 1.80- |
| | 1.71 (m, 4H), 1.64 (td, J = 11.6, 2.9 Hz, 2H). |
| 363 | LCMS (ESI, m/z): 423 [M + H]. |
| | ¹H NMR (400 MHz, DMSO-d₆) δ 9.62 (s, 1H), 9.10 (t, |
| | J = 2.7 Hz, 1H), 8.52 (d, J = 9.5 Hz, 1H), 8.33 (d, J = 8.5 |
| | Hz, 1H), 8.06 (d, J = 8.4 Hz, 1H), 7.40 (d, J = 9.4 Hz, 1H), |
| | 6.43 (dd, J = 5.7, 2.7 Hz, 1H), 5.65 (tt, J = 11.0, 5.9 Hz, |
| | 1H), 4.03 (s, 2H), 2.41 (ddd, J = 13.6, 5.7, 2.9 Hz, 2H), |
| | 2.08-1.84 (m, 6H). |
| 368 | LCMS (ESI, m/z): 447 [M + H]. |
| | ¹H NMR (400 MHz, DMSO-d₆) δ 9.70 (s, 1H), 8.98 (d, |
| | J = 1.1 Hz, 1H), 8.62-8.55 (m, 2H), 8.46-8.37 (m, 2H), |
| | 7.40 (d, J = 9.3 Hz, 1H), 5.64 (tt, J = 10.9, 5.9 Hz, 1H), |
| | 4.03 (s, 3H), 3.53 (s, 2H), 2.21 (dt, J = 12.1, 4.6 Hz, 2H), |
| | 1.76 (d, J = 2.5 Hz, 4H), 1.64 (td, J = 11.5, 3.0 Hz, 2H). |
| 403 | LCMS (ESI, m/z): 373 [M + H]. |
| | ¹H NMR (400 MHz, DMSO-d₆) δ 9.60 (s, 1H), 8.53 (d, |
| | J = 9.5 Hz, 1H), 8.26 (d, J = 8.3 Hz, 1H), 7.85 (d, J = 8.2 |
| | Hz, 1H), 7.37 (d, J = 9.4 Hz, 1H), 5.61 (tt, J = 11.0, 5.9 |
| | Hz, 1H), 3.52 (s, 2H), 2.19 (ddd, J= 11.8, 5.7, 2.8 Hz, 2H), |
| | 1.79-1.70 (m, 4H), 1.62 (td, J = 11.5, 3.0 Hz, 2H). |
| 408 | LCMS (ESI, m/z): 431 [M + H]. |
| | ¹H NMR (400 MHz, DMSO-d₆) δ 9.70-9.61 (m, 2H), |
| | 8.62 (d, J = 9.4 Hz, 1H), 8.43 (d, J = 7.9 Hz, 1H), 8.15 (d, |
| | J = 2.1 Hz, 1H), 8.08 (d, J = 7.8 Hz, 1H), 7.40 (d, J = 9.3 |
| | Hz, 1H), 5.64 (tt, J = 10.9, 5.9 Hz, 1H), 3.53 (s, 2H), 2.75 |
| | (s, 3H), 2.21 (ddd, J = 12.4, 5.9, 2.7 Hz, 2H), 1.76 (s, 4H), |
| | 1.64 (td, J = 11.4, 2.9 Hz, 2H). |

### Example 89: Synthesis of Compound 350

### Synthesis of Intermediate C37

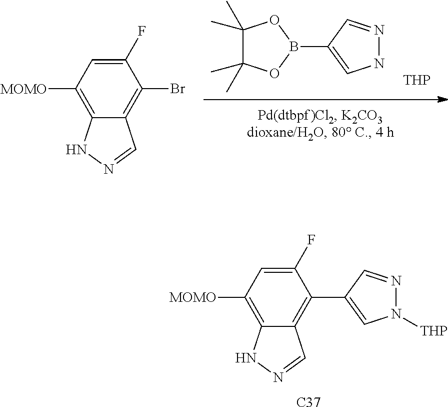

To a stirred solution of 4-bromo-5-fluoro-7-(methoxymethoxy)-1H-indazole (4.0 g, 14.54 mmol, 1.00 equiv) and 1-(oxan-2-yl)-4-(4,4,5,5-tetramethyl-1,3,2-dioxaborolan-2-yl)pyrazole (4.85 g, 17.44 mmol, 1.20 equiv) in dioxane (40.0 mL) and H₂O (8.0 mL) were added Pd(dtbpf)Cl₂ (1.90 g, 2.90 mmol, 0.20 equiv) and K₂CO₃ (6.03 g, 43.62 mmol, 3.00 equiv) dropwise for 4 h at 80° C. under N₂ atmosphere. The mixture was allowed to cool down to 25° C. The resulting mixture was extracted with ethyl acetate (3×50 mL). The combined organic layers were washed with H₂O (1×50 mL), dried over anhydrous Na₂SO₄. The residue was purified by silica gel column chromatography, eluted with PE/EA (3:1) to afford 5-fluoro-7-(methoxymethoxy)-4-[1-(oxan-2-yl)pyrazol-4-yl]-1H-indazole (3.0 g, C37) as a solid that was taken to the next step without further purification. LCMS (ES, m/z): 347 [M+H]

### Synthesis of Intermediate C38

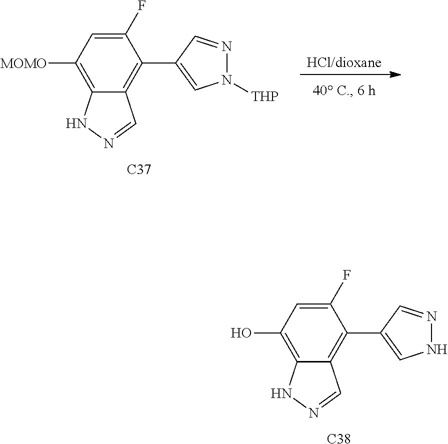

A solution 5-fluoro-7-(methoxymethoxy)-4-[1-(oxolan-2-yl)pyrazol-4-yl]-1H-indazole (2.6 g, 7.82 mmol, 1.00 equiv) and HCl(gas)in 1,4-dioxane (6.0 mL, 197.47 mmol, 25.24 equiv) in MeOH (6.0 mL) was stirred for 6 hr at 40° C. The resulting mixture was concentrated under reduced pressure. The residue was purified by reverse flash chromatography with the following conditions: column, silica gel; mobile phase, MeCN in water (10 mmol/L NH₄HCO₃), 10% to 50% gradient in 10 min; detector, UV 254 nm to afford 5-fluoro-4-(1H-pyrazol-4-yl)-1H-indazol-7-ol (1.62 g, C38) as a solid. LCMS (ES, m/z): 219 [M+H]

### Synthesis of Intermediate C39

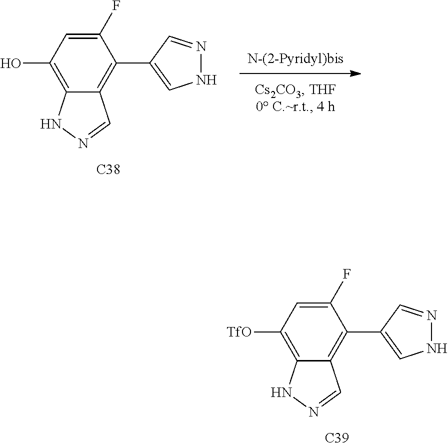 [SMILES: Oc1cc(F)c(-c2cn[nH]c2)c2cn[nH]c12 ; "N-(2-Pyridyl)bis" ; "C38" ; O=S(=O)(Oc1cc(F)c(-c2cn[nH]c2)c2cn[nH]c12)C(F)(F)F ; "C39"]

A solution of 5-fluoro-4-(1H-pyrazol-4-yl)-1H-indazol-7-ol (1.42 g, 6.50 mmol, 1.00 equiv) in THE (10 mL) was treated with Cs₂CO₃ (4.24 g, 13.01 mmol, 2.00 equiv) for 30 min at 0° C. under nitrogen atmosphere followed by the addition of 1,1,1-trifluoro-N-phenyl-N-trifluoromethanesulfonylmethanesulfonamide (2.56 g, 7.15 mmol, 1.10 equiv) dropwise for 3.5 h at 25° C. The resulting mixture was extracted with ethyl acetate (3×50 mL). The combined organic layers were washed with H₂O (1×50 mL) and dried over anhydrous Na₂SO₄. After filtration, the filtrate was concentrated under reduced pressure. The residue was purified by reverse flash chromatography with the following conditions: column, silica gel; mobile phase, MeCN in water (10 mmol/L NH₄HCO₃), 10% to 50% gradient in 10 min; detector, UV 254 nm. This resulted in 5-fluoro-4-(1H-pyrazol-4-yl)-1H-indazol-7-yl trifluoromethanesulfonate (1.22 g, 46.0%) as a solid that was taken to the next step without further purification. LCMS (ES, m/z): 351 [M+H]⁺

### Synthesis of Intermediate C40

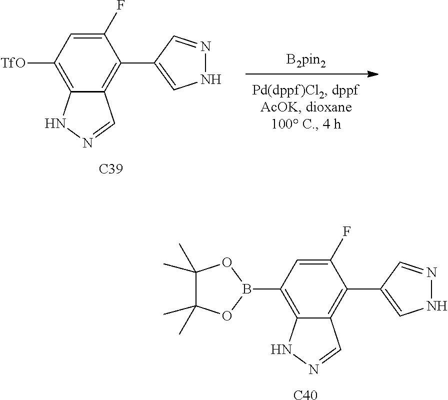 [SMILES: O=S(=O)(Oc1cc(F)c(-c2cn[nH]c2)c2cn[nH]c12)C(F)(F)F ; "B2pin2" ; "C39" ; CC1(C)OB(c2cc(F)c(-c3cn[nH]c3)c3cn[nH]c23)OC1(C)C ; "C40"]

To a stirred solution 5-fluoro-4-(1H-pyrazol-4-yl)-1H-indazol-7-yl trifluoromethanesulfonate (800.0 mg, 2.284 mmol, 1.00 equiv) and bis(pinacolato)diboron (870.0 mg, 3.42 mmol, 1.50 equiv) in dioxane (8.0 mL) were added Pd(dppf)Cl₂ (334.2 mg, 0.45 mmol, 0.20 equiv) and dppf (504.6 mg, 0.91 mmol, 0.40 equiv) and potassium acetate (672.5 mg, 6.85 mmol, 3.00 equiv) dropwise for 4 hr at 100° C. under N₂ atmosphere. The mixture was allowed to cool down to 25° C. The resulting mixture was extracted with ethyl acetate (2×50 mL). The combined organic layers were washed with H₂O (1×50 mL), dried over anhydrous Na₂SO₄. After filtration, the filtrate was concentrated under reduced pressure. The residue was purified by silica gel column chromatography, eluted with PE/EA (1:1) to afford 5-fluoro-4-(1H-pyrazol-4-yl)-7-(4,4,5,5-tetramethyl-1,3,2-dioxaborolan-2-yl)-1H-indazole (600.0 mg, C40) as a solid that was taken to the next step without further purification. LCMS (ES, m/z): 329 [M+H]⁺

### Synthesis of Intermediate C41

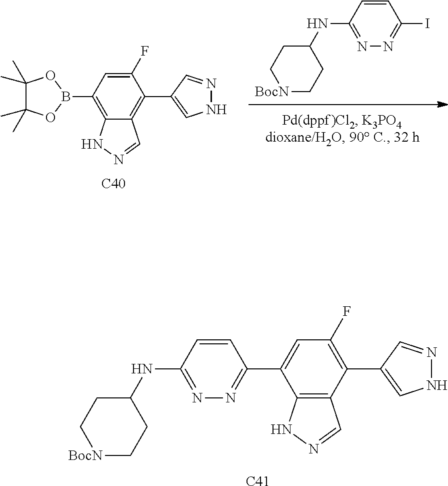 [SMILES: CC1(C)OB(c2cc(F)c(-c3cn[nH]c3)c3cn[nH]c23)OC1(C)C ; CC(C)(C)OC(=O)N1CCC(Nc2ccc(I)nn2)CC1 ; "C40" ; CC(C)(C)OC(=O)N1CCC(Nc2ccc(-c3cc(F)c(-c4cn[nH]c4)c4cn[nH]c34)nn2)CC1 ; "C41"]

To a stirred solution of 5-fluoro-4-(1H-pyrazol-4-yl)-7-(4,4,5,5-tetramethyl-1,3,2-dioxaborolan-2-yl)-1H-indazole (100.0 mg, 0.30 mmol, 1.00 equiv) and tert-butyl 4-[(6-iodopyridazin-3-yl)amino]piperidine-1-carboxylate (147.8 mg, 0.36 mmol, 1.20 equiv) in dioxane (2.0 mL, 23.60 mmol, 77.47 equiv) and H₂O (0.40 mL, 22.203 mmol, 72.86 equiv) were added Pd(dppf)Cl₂ (44.60 mg, 0.061 mmol, 0.20 equiv) and K₃PO₄ (194.0 mg, 0.91 mmol, 3 equiv) dropwise for 32 hr at 90° C. under N2 atmosphere. The mixture was allowed to cool down to 25° C. The resulting mixture was extracted with ethyl acetate (2×30 mL). The combined organic layers were washed with H₂O (1×30 mL), dried over anhydrous Na₂SO₄. After filtration, the filtrate was concentrated under reduced pressure. The residue was purified by silica gel column chromatography, eluted with PE/EA (5:1) to afford tert-butyl 4-({6-[5-fluoro-4-(1H-pyrazol-4-yl)-1H-indazol-7-yl] pyridazin-3-yl}amino)piperidine-1-carboxylate (95.0 mg, C41) as a solid that was taken to the next step without further purification. LCMS (ES, m/z): 479 [M+H]

### Synthesis of Compound 350

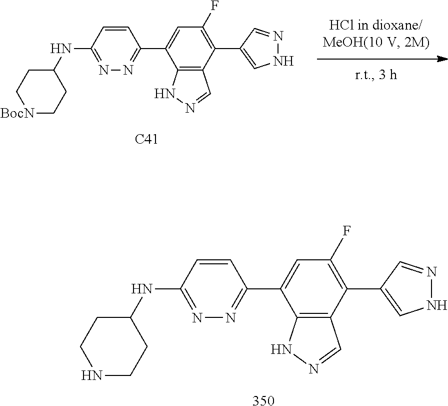 [SMILES: CC(C)(C)OC(=O)N1CCC(Nc2ccc(-c3cc(F)c(-c4cn[nH]c4)c4cn[nH]c34)nn2)CC1 ; "C41" ; Fc1cc(-c2ccc(NC3CCNCC3)nn2)c2[nH]ncc2c1-c1cn[nH]c1 ; "350"]

A solution tert-butyl 4-({6-[5-fluoro-4-(1H-pyrazol-4-yl)-1H—in dazol-7-yl]pyridazin-3-yl}amino)piperidine-1-carboxylate (60.0 mg, 0.12 mmol, 1.00 equiv) and HCl(gas)in 1,4-dioxane (2.0 mL, 65.82 mmol, 524.98 equiv) in methanol (2.0 mL, 49.39 mmol, 393.97 equiv) was stirred for 3 hr at 25° C. The resulting mixture was concentrated under reduced pressure. The crude product was purified by preparative HPLC (Condition 8, Gradient 5). This resulted in 6-[5-fluoro-4-(1H-pyrazol-4-yl)-1H-indazol-7-yl]-N-(piperidin-4-yl)pyridazin-3-amine hydrochloride (14.4 mg, Compound 350) as a solid. LCMS (ES, m/z): 379 [M+H] ¹H NMR (400 MHz, DMSO-d₆) δ 13.14 (s, 1H), 9.53 (s, 1H), 8.97 (s, 2H), 8.62 (s, 1H), 8.54 (s, 1H), 8.37-8.32 (m, 2H), 8.06 (d, J=12.2 Hz, 1H), 7.69 (s, 1H), 4.23 (s, 1H), 3.42 (d, J=12.7 Hz, 2H), 3.05 (s, 2H), 2.20 (d, J=13.2 Hz, 2H), 1.86 (q, J=10.5 Hz, 2H). ¹⁹F NMR (376 MHz, DMSO-d₆) δ-124.63.

### Example 90: Synthesis of Compound 354

### Synthesis of Intermediate C42

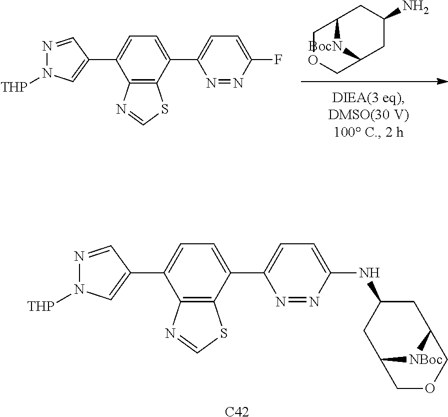

A solution of 7-(6-fluoropyridazin-3-yl)-4-[1-(oxan-2-yl)pyrazol-4-yl]-1,3-benzothiazole (200 mg, 0.514 mmol, 1.00 eq.), tert-butyl (endo)-7-amino-3-oxa-9-azabicyclo[3.3.1]nonane-9-carboxylate (125 mg, 0.514 mmol, 1 eq.) and N,N-diisopropylethylamine (199 mg, 1.542 mmol, 3.00 eq.) in dimethyl sulfoxide (6 mL) was stirred for 2 hr at 100° C. The mixture was allowed to cool to room temperature. The resulting mixture was poured into water (30 mL). The precipitated solids were collected by filtration and washed with hexane (2×30 mL). The resulting mixture was concentrated under vacuum to afford tert-butyl (endo)-7-[(6-{4-[1-(oxan-2-yl)pyrazol-4-yl]-1,3-benzothiazol-7-yl}pyridazin-3-yl)amino]-3-oxa-9-azabicyclo[3.3.1]nonane-9-carboxylate (200 mg, C42) as solid that was taken to the next step without further purification. LCMS (ESI, m/z): 604 [M+H].

### Synthesis of Compound 354

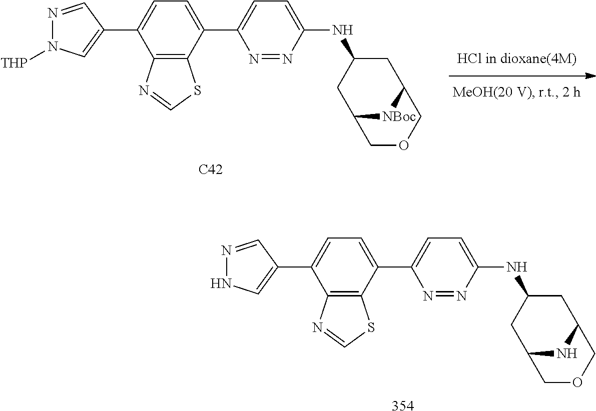

A mixture of tert-butyl (endo)-7-[(6-{4-[1-(oxan-2-yl)pyrazol-4-yl]-1,3-benzothiazol-7-yl}pyridazin-3-yl)amino]-3-oxa-9-azabicyclo[3.3.1]nonane-9-carboxylate (80 mg, 0.130 mmol, 1.00 eq.) in hydrochloric acid(gas)in 1,4-dioxane (1.6 mL) and methanol (1.6 mL) was stirred for 2 hr at room temperature. The resulting mixture was concentrated under vacuum. The crude product was purified by preparative HPLC (Condition 7, Gradient 2) to afford (endo)-N-{6-[4-(1H-pyrazol-4-yl)-1,3-benzothiazol-7-yl]pyridazin-3-yl}-3-oxa-9-azabicyclo[3.3.1]nonan-7-amine (15.4 mg, Compound 354) as a solid.

Compounds 353, 357, and 358 were synthesized using an analogous method. Data for compounds 353, 354, 357, and 358 are shown in Table 8 below.

TABLE 8
Exemplary compounds

| Compound | Analysis data |
|---|---|
| 353 | LCMS (ESI, m/z): 420 [M + H]. |
| | ¹H NMR (400 MHz, DMSO-d₆) δ 13.07 (s, 1H), 9.51 (s, 1H), 8.66 |
| | (s, 1H), 8.40 (s, 1H), 8.13 (d, J = 9.6 Hz, 1H), 8.04 (d, J = 8.1 Hz, |
| | 1H), 7.97 (d, J = 8.0 Hz, 1H), 6.94 (d, J = 9.5 Hz, 1H), 6.85 (d, J = |
| | 8.2 Hz, 1H), 5.24 (s, 1H), 3.83 (d, J = 10.8 Hz, 2H), 3.75 (dt, J = |
| | 10.9, 2.3 Hz, 2H), 2.91 (s, 2H), 2.21 (dd, J = 12.3, 5.7 Hz, 2H), 1.65 |
| | (td, J = 12.2, 4.0 Hz, 2H). |
| 354 | LCMS (ESI, m/z): 420 [M + H]. |
| | ¹H NMR (400 MHz, DMSO-d₆) δ 13.08 (s, 1H), 9.50 (s, 1H), 8.64 |
| | (s, 1H), 8.44 (s, 1H), 8.17 (d, J = 9.6 Hz, 1H), 8.07 (d, J = 8.1 Hz, |
| | 1H), 7.97 (d, J = 7.9 Hz, 1H), 7.22 (d, J = 9.3 Hz, 1H), 7.02 (d, J = |
| | 9.5 Hz, 1H), 4.52 (s, 1H), 3.76-3.60 (m, 4H), 2.94 (d, J = 7.7 Hz, |
| | 2H), 2.34-2.24 (m, 2H), 1.59 (ddd, J = 13.6, 6.2, 2.4 Hz, 2H). |
| 357 | LCMS (ESI, m/z): 421 [M + H]. |
| | ¹H NMR (400 MHz, DMSO-d₆) δ 13.12 (s, 1H), 9.55 (s, 1H), 8.71 |
| | (s, 1H), 8.53 (d, J = 9.4 Hz, 1H), 8.44 (s, 1H), 8.23 (d, J = 8.0 Hz, |
| | 1H), 8.04 (d, J = 7.9 Hz, 1H), 7.34 (d, J = 9.3 Hz, 1H), 6.41 (tt, J = |
| | 11.2, 6.1 Hz, 1H), 3.81 (d, J = 10.9 Hz, 2H), 3.73 (d, J = 10.7 Hz, |
| | 2H), 3.06-3.00 (m, 2H), 2.38 (dd, J = 12.0, 6.4 Hz, 2H), 1.85 (td, |
| | J = 11.7, 4.7 Hz, 2H). |
| 358 | LCMS (ESI, m/z): 421 [M + H]. |
| | ¹H NMR (400 MHz, DMSO-d₆) δ 13.13 (s, 1H), 9.55 (s, 1H), 8.69 |
| | (s, 1H), 8.54 (d, J = 9.4 Hz, 1H), 8.46 (s, 1H), 8.24 (d, J = 8.1 Hz, |
| | 1H), 8.04 (d, J = 7.9 Hz, 1H), 7.33 (d, J = 9.3 Hz, 1H), 5.66-5.54 |
| | (m, 1H), 3.57 (dd, J = 10.7, 2.1 Hz, 2H), 3.52 (d, J = 10.5 Hz, 2H), |
| | 3.06 (d, J = 9.3 Hz, 2H), 1.73 (ddd, J = 12.3, 8.6, 2.9 Hz, 2H). |

### Example 91: Synthesis of Compound 356

### Synthesis of Intermediate C43

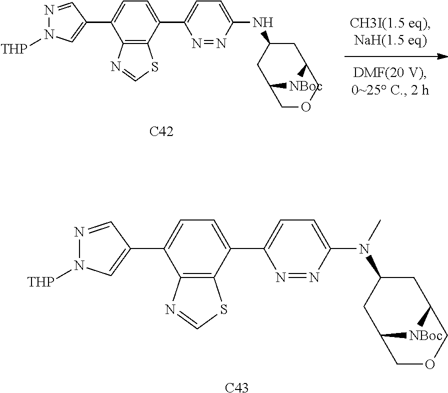

To a stirred solution of tert-butyl (endo)-7-[(6-{4-[1-(oxan-2-yl)pyrazol-4-yl]-1,3-benzothiazol-7-yl}pyridazin-3-yl)amino]-3-oxa-9-azabicyclo[3.3.1]nonane-9-carboxylate (120 mg, 0.195 mmol, 1.00 eq.) in dimethyl formamide (2.4 mL) were added sodium hydride (12 mg, 0.292 mmol, 1.5 eq.) in portions at 0° C. To the above mixture was added methyl iodide (41 mg, 0.292 mmol, 1.5 eq.) dropwise at 0° C. The resulting mixture was stirred for additional 1 hr at room temperature. The resulting mixture was poured into water (20 mL). The resulting mixture was extracted with ethyl acetate (3×60 mL). The combined organic layers were washed with brine (2×20 mL), dried over anhydrous sodium sulfate. After filtration, the filtrate was concentrated under reduced pressure. The residue was purified by silica gel column chromatography, eluted with petroleum ether/ethyl acetate(1:1) to afford tert-butyl (endo)-7-[methyl(6-{4-[1-(oxan-2-yl)pyrazol-4-yl]-1,3-benzothiazol-7-yl}pyridazin-3-yl)amino]-3-oxa-9-azabicyclo[3.3.1]nonane-9-carboxylate (80 mg, c43) as a solid that was taken to the next step without further purification. LCMS (ESI, m/z): 618 [M+H].

### Synthesis of Compound 356

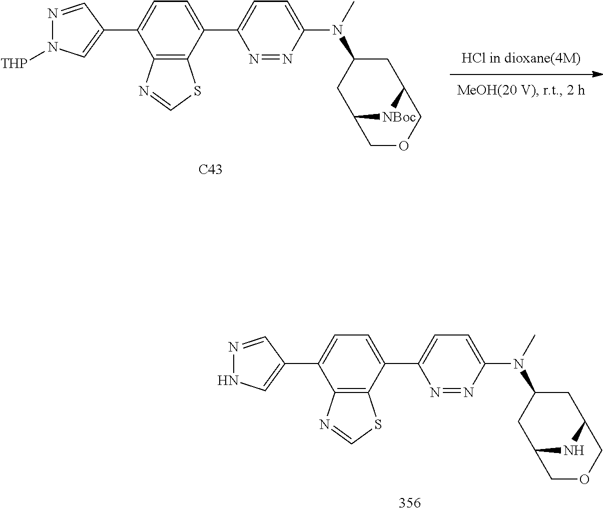

A mixture of tert-butyl (endo)-7-[methyl(6-{4-[1-(oxan-2-yl)pyrazol-4-yl]-1,3-benzothiazol-7-yl}pyridazin-3-yl)amino]-3-oxa-9-azabicyclo[3.3.1]nonane-9-carboxylate (80 mg, 0.127 mmol, 1.00 eq.) in hydrochloric acid(gas)in 1,4-dioxane (1.6 mL) and methanol (1.6 mL) was stirred for 2 hr at room temperature. The resulting mixture was concentrated under vacuum. The crude product was purified by Prep-HPLC (Condition 7, Gradient 1) to afford (endo)-N-methyl-N-{6-[4-(1H-pyrazol-4-yl)-1,3-benzothiazol-7-yl]pyridazin-3-yl}-3-oxa-9-azabicyclo[3.3.1]nonan-7-amine (31.4 mg, 57%) as a solid. LCMS (ESI, m/z): 434 [M+H].

Compound 355 was synthesized using an analogous method with the use of tert-butyl (exo)-7-[(6-{4-[1-(oxan-2-yl)pyrazol-4-yl]-1,3-benzothiazol-7-yl}pyridazin-3-yl)amino]-3-oxa-9-azabicyclo[3.3.1]nonane-9-carboxylate. LCMS (ESI, m/z): 434 [M+H]. ¹H NMR (400 MHz, DMSO-d₆) δ 13.09 (s, 1H), 9.51 (s, 1H), 8.63 (s, 1H), 8.44 (s, 1H), 8.27 (d, J=9.8 Hz, 1H), 8.09 (d, J=8.0 Hz, 1H), 7.99 (d, J=7.9 Hz, 1H), 7.16 (d, J=9.8 Hz, 1H), 5.95 (s, 1H), 3.85 (d, J=10.9 Hz, 2H), 3.76 (d, J=10.9 Hz, 2H), 3.00 (s, 3H), 2.98 (d, J=15.5 Hz, 2H), 2.11-1.99 (m, 2H), 1.80 (dd, J=12.6, 5.8 Hz, 2H).

### Example 92: Synthesis of Compound 359

### Synthesis of Intermediate C44

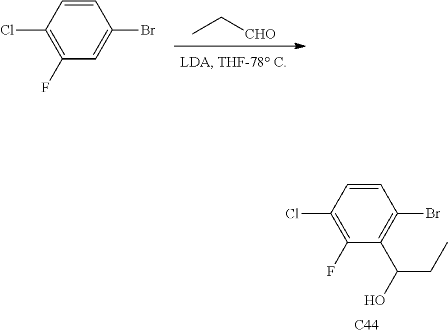

To a stirred solution of diisopropylamine (14.49 g, 143.22 mmol, 2.0 equiv) in THE (600 mL) was added n-BuLi (60.30 mL, 150.39 mmol, 2.1 equiv) dropwise at −80° C. under nitrogen atmosphere. The resulting mixture was stirred for 1 h at −30° C. under nitrogen atmosphere. The mixture was added 4-bromo-1-chloro-2-fluorobenzene (15.00 g, 71.61 mmol, 1.0 equiv) at -78° C. and stirred at −30° C. for 1 hr. The mixture was added propionaldehyde (8.32 g, 143.22 mmol, 2.0 equiv) at −78° C. and stirred at −30° C. for 1 hr. The reaction was quenched with saturated aqueous NH₄Cl at −30° C. The resulting mixture was extracted with EtOAc (3×600 mL). The combined organic layers were washed with water (2×800 mL) and dried over anhydrous Na₂SO₄. After filtration, the filtrate was concentrated under reduced pressure. The residue was purified by silica gel column chromatography, eluted with PE/EA (3:1) to afford 1-(6-bromo-3-chloro-2-fluorophenyl)ethanol (9.5 g, C44) as a solid that was taken to the next step without further purification. ¹H NMR (400 MHz, Chloroform-d) δ 7.33 (dd, J=8.7, 1.7 Hz, 1H), 7.21 (dd, J=8.6, 7.6 Hz, 1H), 5.11 (dddd, J=9.3, 7.9, 6.6, 1.1 Hz, 1H), 2.39 (dd, J=9.3, 4.4 Hz, 1H), 2.13-1.81 (m, 2H), 1.06-0.93 (m, 3H).

### Synthesis of Intermediate C45

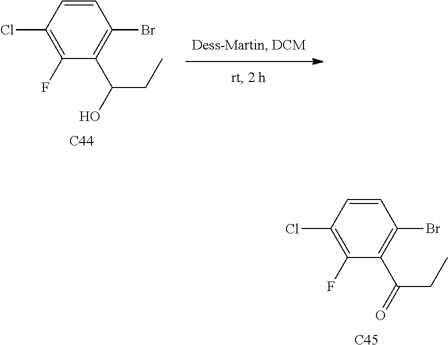

A mixture of 1-(6-bromo-3-chloro-2-fluorophenyl)propan-1-ol (9.5 g, 35.53 mmol, 1.0 equiv) and Dess-Martin periodinane (22.59 g, 53.30 mmol, 1.5 equiv) in DCM (200 mL) was stirred for 2 hr at room temperature. The reaction was quenched with saturated aqueous Na₂CO₃ at room temperature. The resulting mixture was washed with water (3×200 mL). The organic layer was dried over anhydrous Na₂SO₄. After filtration, the filtrate was concentrated under reduced pressure. The residue was purified by silica gel column chromatography, eluted with PE/EA (10:1) to afford 1-(6-bromo-3-chloro-2-fluorophenyl)propan-1-one (10.0 g, C45) as an oil that was taken to the next step without further purification.

### Synthesis of Intermediate C46

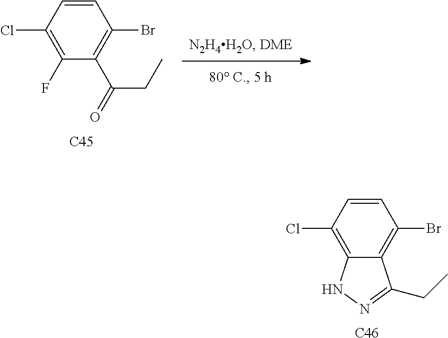

To a stirred solution of 1-(6-bromo-3-chloro-2-fluorophenyl)propan-1-one (10.0 g, 37.88 mmol, 1.0 equiv) in dimethoxymethane (100 mL) was added N₂H₄·H₂O (50 mL) at room temperature. The resulting mixture was stirred for 5 hr at 80° C. The resulting mixture was diluted with water (400 mL) and extracted with ethyl acetate (2×400 mL). The combined organic layers were washed with water (600 mL) and brine (600 mL), dried over anhydrous Na₂SO₄. After filtration, the filtrate was concentrated under reduced pressure. The residue was purified by silica gel column chromatography, eluted with PE/EA(2/1) to afford 4-bromo-7-chloro-3-ethyl-1H-indazole (5.6 g, C46) as a solid that was taken to the next step without further purification.

### Synthesis of Intermediate C47

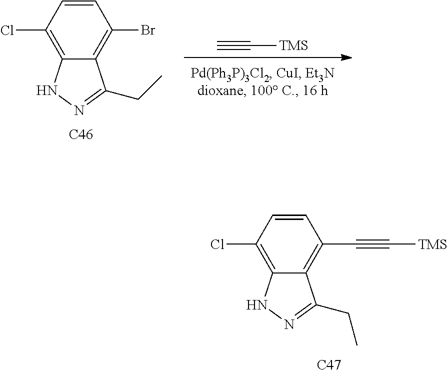

To a stirred solution of 4-bromo-7-chloro-3-ethyl-1H-indazole (5.1 g, 19.77 mmol, 1.0 equiv) and trimethylsilylacetylene (2.93 g, 29.655 mmol, 1.5 equiv) in dioxane (100 mL) was added Et₃N (5.99 g, 59.310 mmol, 3.0 equiv) and CuI (0.38 g, 1.977 mmol, 0.1 equiv) and Pd(PPh₃)₂Cl₂ (1.61 g, 1.977 mmol, 0.1 equiv) at rt under N₂ atmosphere. The resulting mixture was stirred for 16 hr at 100° C. under N₂ atmosphere. The resulting mixture was diluted with water (200 mL) and extracted with EA (2×200 mL). The combined organic layers were washed with water (400 mL) and brine (400 mL), dried over anhydrous Na₂SO₄. After filtration, the filtrate was concentrated under reduced pressure. The residue was purified by silica gel column chromatography, eluted with PE/EA (2/1) to afford 7-chloro-3-ethyl-4-((trimethylsilyl)ethynyl)-1H-indazole (4.72 g, C47) as a solid that was taken to the next step without further purification. LCMS (ES, m/z): 277 [M+H]⁺

### Synthesis of Intermediate C48

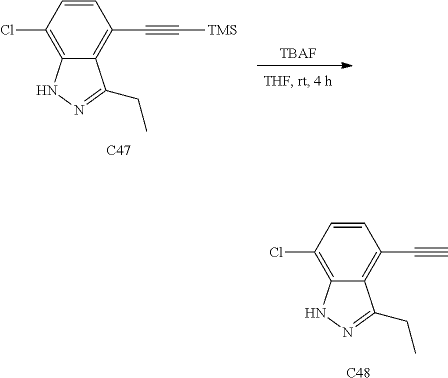 [SMILES: CCc1n[nH]c2c(Cl)ccc(C#C[Si](C)(C)C)c12 ; "TBAF" ; "C47" ; C#Cc1ccc(Cl)c2[nH]nc(CC)c12 ; "C48"]

To a stirred solution of 7-chloro-3-ethyl-4-((trimethylsilyl)ethynyl)-1H-indazole (3.50 g, 12.681 mmol, 1.0 equiv) in THE (70 mL) was added TBAF (0.66 g, 2.536 mmol, 0.2 equiv) at room temperature. The resulting mixture was stirred for 4 hr at room temperature. The resulting mixture was diluted with water (100 mL) and extracted with EA (2×100 mL). The combined organic layers were washed with water (200 mL) and brine (200 mL), dried over anhydrous Na₂SO₄. After filtration, the filtrate was concentrated under reduced pressure. The residue was purified by silica gel column chromatography, eluted with PE/EA (2/1) to afford 7-chloro-3-ethyl-4-ethynyl-1H-indazole (1.9 g, C48) as a solid that was taken to the next step without further purification. LCMS (ES, m/z): 205 [M+H]⁺

### Synthesis of Intermediate C49

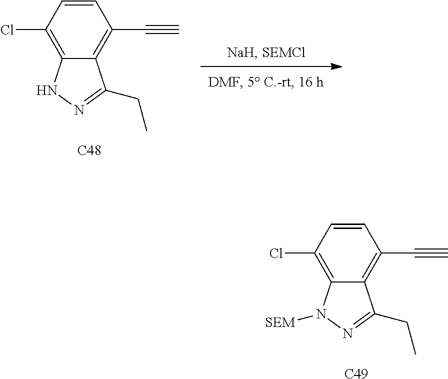

To a stirred mixture of 7-chloro-3-ethyl-4-ethynyl-1H-indazole (2.6 g, 12.704 mmol, 1.0 equiv) in THE (52.0 mL) was added NaH (609.7 mg, 25.408 mmol, 2.0 equiv) at 5° C. under nitrogen atmosphere. The resulting mixture was stirred for 30 min at 5° C. under nitrogen atmosphere. To the above mixture was added 2-(trimethylsilyl)ethoxymethyl chloride (2541.6 mg, 15.245 mmol, 1.2 equiv) dropwise at 5° C. The resulting mixture was stirred for an additional 16 hr at room temperature. The reaction was quenched with ice water at 0° C. The resulting mixture was extracted with EtOAc (1×100 mL). The combined organic layers were washed with water (1×100 mL), dried over anhydrous Na₂SO₄. After filtration, the filtrate was concentrated under reduced pressure. The residue was purified by silica gel column chromatography, eluted with PE/EA (5:1) to afford 7-chloro-3-ethyl-4-ethynyl-1-{[2-(trimethylsilyl)ethoxy]methyl}indazole (750 mg, C49) as a solid that was taken to the next step without further purification. LCMS (ES, m/z): 335 [M+H]⁺

### Synthesis of Intermediate C50

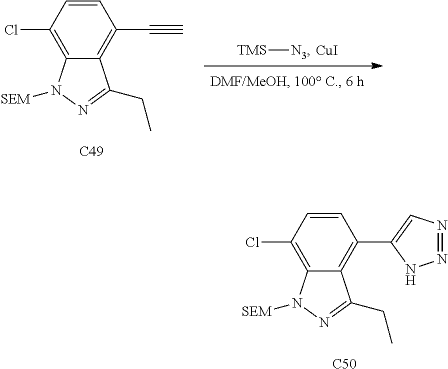

To a stirred mixture of 7-chloro-3-ethyl-4-ethynyl-1-{[2-(trimethylsilyl)ethoxy]methyl}indazole (700.0 mg, 2.090 mmol, 1.0 equiv) in DMF (6.3 mL) and MeOH (0.7 mL) were added CuI (39.8 mg, 0.209 mmol, 0.1 equiv) and trimethylsilyl azide (481.5 mg, 4.180 mmol, 2.0 equiv) at room temperature. The resulting mixture was stirred for 6 h at 100° C. under nitrogen atmosphere. The resulting mixture was diluted with water (20 mL). The resulting mixture was extracted with ethyl acetate (2×20 mL). The combined organic layers were washed with water (2×20 mL), dried over anhydrous Na₂SO₄. After filtration, the filtrate was concentrated under reduced pressure. The residue was purified by silica gel column chromatography, eluted with PE/EA (3:1) to afford 7-chloro-3-ethyl-4-(3H-1,2,3-triazol-4-yl)-1-{[2-(trimethylsilyl)ethoxy]methyl}indazole (380 mg, C50) as a solid that was taken to the next step without further purification. LCMS (ES, m/z): 378 [M+H]⁺

### Synthesis of Intermediate C51

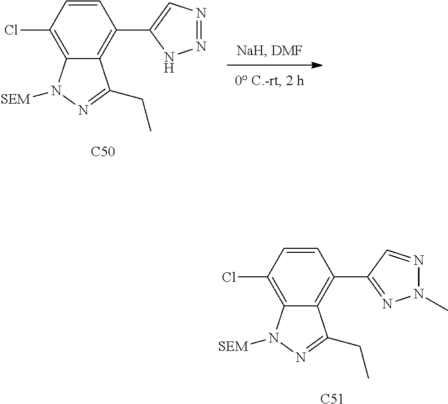

To a stirred mixture of 7-chloro-3-ethyl-4-(3H-1,2,3-triazol-4-yl)-1-{[2-(trimethylsilyl)ethoxy]methyl}indazole (370.0 mg, 0.979 mmol, 1.0 equiv) in DMF (5.2 mL) was added NaH (35.2 mg, 1.468 mmol, 1.5 equiv) at 0° C. The resulting mixture was stirred for 10 min at 0° C. To the above mixture was added methyl iodide (208.4 mg, 1.468 mmol, 1.5 equiv) dropwise at 0° C. The resulting mixture was stirred for additional 2 hr at room temperature. The reaction was quenched with ice water The resulting mixture was extracted with EtOAc (2×10 mL). The combined organic layers were washed with water (1×20 mL), dried over anhydrous Na₂SO₄. After filtration, the filtrate was concentrated under reduced pressure. The residue was purified by silica gel column chromatography, eluted with PE/EA (3:1) to afford 7-chloro-3-ethyl-4-(2-methyl-1,2,3-triazol-4-yl)-1-{[2-(trimethylsilyl)ethoxy]methyl}indazole (250 mg, C51) as a solid that was taken to the next step without further purification. LCMS (ES, m/z): 392 [M−H]⁻

### Synthesis of Intermediate C52

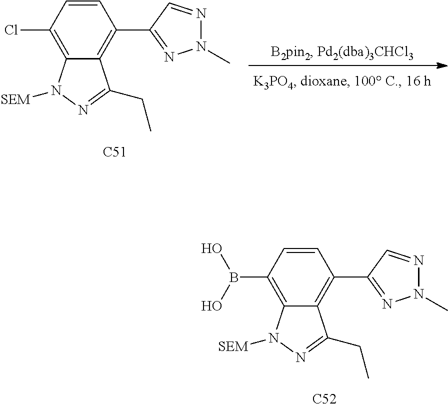 [SMILES: CCc1nn(COCC[Si](C)(C)C)c2c(Cl)ccc(-c3cnn(C)n3)c12 ; "B2pin2, Pd2(dba)3CHCl3" ; "C51" ; CCc1nn(COCC[Si](C)(C)C)c2c(B(O)O)ccc(-c3cnn(C)n3)c12 ; "C52"]

To a stirred mixture of 7-chloro-3-ethyl-4-(2-methyl-1,2,3-triazol-4-yl)-1-{[2-(trimethylsilyl)ethoxy]methyl}indazole (200.0 mg, 0.510 mmol, 1.0 equiv) and B₂pin2 (194.3 mg, 0.765 mmol, 1.5 equiv) in dioxane (4 mL) were added potassium acetate (100.1 mg, 1.020 mmol, 2.0 equiv) at room temperature under nitrogen atmosphere. The resulting mixture was stirred for 16 h at 100° C. under nitrogen atmosphere. The resulting mixture was filtered, the filter cake was washed with EA (4 mL). The filtrate was concentrated under reduced pressure. The resulting mixture was concentrated under vacuum. The residue was purified by silica gel column chromatography, eluted with PE/EA (1/1) to afford 3-ethyl-4-(2-methyl-1,2,3-triazol-4-yl)-1-{[2-(trimethylsilyl)ethoxy]methyl}indazol-7-ylboronic acid (140 mg, C52) as a solid that was taken to the next step without further purification. LCMS (ES, m/z): 402 [M+H]⁺

### Synthesis of Intermediate C53

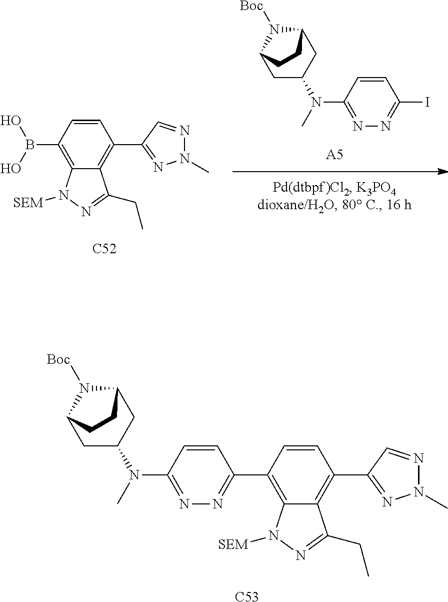

To a stirred mixture of 3-ethyl-4-(2-methyl-1,2,3-triazol-4-yl)-1-{[2-(trimethylsilyl)ethoxy]methyl}indazol-7-ylboronic acid (120.0 mg, 0.299 mmol, 1.0 equiv) and tert-butyl (1R,3S,5S)-3-[(6-iodopyridazin-3-yl)(methyl)amino]-8-azabicyclo[3.2.1]octane-8-carboxylate (132.8 mg, 0.299 mmol, 1.0 equiv) in dioxane (1.6 mL) and H₂O (0.16 mL) were added K₃PO₄ (190.4 mg, 0.897 mmol, 3.0 equiv) and Pd(dppf)Cl₂·CH₂Cl₂ (24.4 mg, 0.030 mmol, 0.1 equiv) at room temperature under nitrogen atmosphere. The resulting mixture was stirred for 16 h at 80 degrees C. under nitrogen atmosphere. The resulting mixture was filtered, the filter cake was washed with ethyl acetate (1×5 mL). The filtrate was concentrated under reduced pressure. The residue was purified by Prep-TLC (PE/EA 1:1) to afford tert-butyl (1R,3S,5S)-3-({6-[3-ethyl-4-(2-methyl-1,2,3-triazol-4-yl)-1-{[2-(trimethylsilyl)ethoxy]methyl}indazol-7-yl]pyridazin-3-yl}(methyl)amino)-8-azabicyclo[3.2.1]octane-8-carboxylate (40 mg, C53) as an oil that was taken to the next step without further purification. LCMS (ES, m/z): 674 [M+H]⁺

### Synthesis of Compound 359

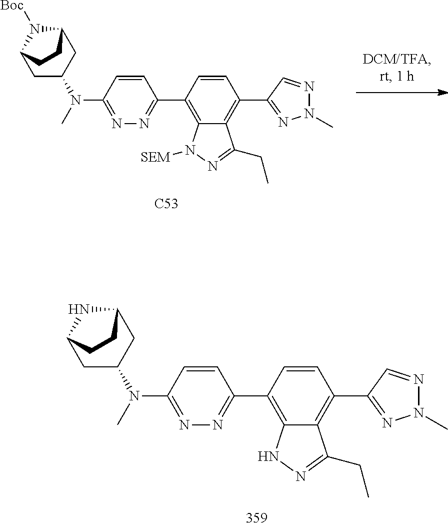

To a stirred mixture of tert-butyl (1R,3S,5S)-3-({6-[3-ethyl-4-(2-methyl-1,2,3-triazol-4-yl)-1-{[2-(trimethylsilyl)ethoxy]methyl}indazol-7-yl]pyridazin-3-yl}(methyl)amino)-8-azabicyclo[3.2.1]octane-8-carboxylate (40 mg, 0.059 mmol, 1.0 equiv) in dichloromethane (1 mL) was added TFA (0.33 mL) dropwise at room temperature. The resulting mixture was stirred for 1 h at room temperature. The resulting mixture was concentrated under reduced pressure. The crude product (mg) was purified by Prep-HPLC (Condition 11, Gradient 2) to afford (1R,3S,5S)-N-{6-[3-ethyl-4-(2-methyl-1,2,3-triazol-4-yl)-1H-indazol-7-yl]pyridazin-3-yl}-N-methyl-8-azabicyclo[3.2.1]octan-3-amine (6.7 mg, Compound 359) as a solid. LCMS (ES, m/z): 444 [M+H]+ 1H NMR (400 MHz, DMSO-d₆) δ 12.82 (s, 1H), 8.16 (d, J=9.8 Hz, 1H), 8.10 (s, 1H), 7.93 (d, J=7.6 Hz, 1H), 7.27 (dd, J=8.6, 5.6 Hz, 2H), 5.05 (d, J=7.6 Hz, 1H), 4.28 (s, 3H), 3.51 (s, 2H), 2.98 (s, 3H), 2.90 (q, J=7.5 Hz, 2H), 1.88-1.78 (m, 2H), 1.78-1.72 (m, 4H), 1.56 (dt, J=11.8, 3.4 Hz, 2H), 0.99 (t, J=7.4 Hz, 3H).

### Example 93: Synthesis of Compound 360

### Synthesis of Intermediate C54

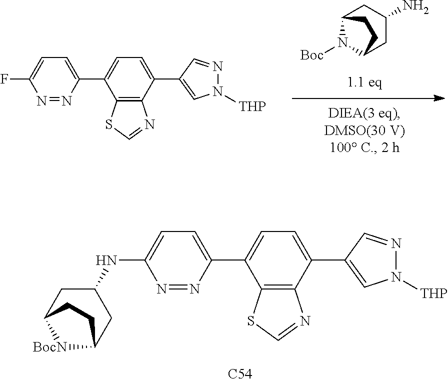

A mixture of 7-(6-fluoropyridazin-3-yl)-4-[1-(oxan-2-yl) pyrazol-4-yl]-1,3-benzothiazole (300 mg, 0.78 mmol, 1.00 equiv), tert-butyl (exo)-3-amino-8-azabicyclo [3.2.1] octane-8-carboxylate (196 mg, 0.86 mmol, 1.10 equiv) and DIEA (305 mg, 2.36 mmol, 3.00 equiv) in DMSO (9.0 mL) was stirred for 2 hr at 100° C. The mixture was allowed to cool down to room temperature. The resulting mixture was diluted with water (20 mL). The precipitated solids were collected by filtration and washed with water (2×20 mL). The resulting mixture was concentrated under vacuum to afford tert-butyl (exo)-3-[(6-{4-[1-(oxan-2-yl) pyrazol-4-yl]-1,3-benzothiazol-7-yl} pyridazin-3-yl) amino]-8-azabicyclo [3.2.1] octane-8-carboxylate (412 mg, C54) as a solid that was taken to the next step without further purification. LCMS (ES, m/z): 588 [M+H]⁺

### Synthesis of Intermediate C55

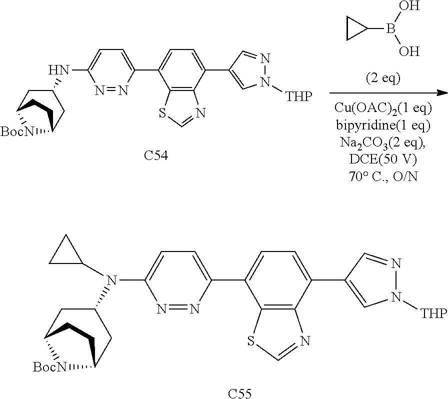

A mixture of tert-butyl (exo)-3-[(6-{4-[1-(oxan-2-yl) pyrazol-4-yl]-1,3-benzothiazol-7-yl}pyridazin-3-yl) amino]-8-azabicyclo [3.2.1] octane-8-carboxylate (410 mg, 0.69 mmol, 1.00 equiv), cyclopropylboronic acid (120 mg, 1.39 mmol, 2.00 equiv), bipyridyl (109 mg, 0.69 mmol, 1.00 equiv), Cu(OAc)₂ (127 mg, 0.69 mmol, 1.00 equiv) and Na₂CO₃ (148 mg, 1.39 mmol, 2.00 equiv) in dichloroethane (20 mL) was stirred over night at 70° C. under oxygen atmosphere. The mixture was allowed to cool down to room temperature. The resulting mixture was diluted with water (50 mL). The resulting mixture was extracted with EA (2×50 mL), dried over anhydrous Na₂SO₄. After filtration, the filtrate was concentrated under reduced pressure. The residue was purified by silica gel column chromatography, eluted with PE/EA (1:2) to afford tert-butyl (exo)-3-[cyclopropyl(6-{4-[1-(oxan-2-yl) pyrazol-4-yl]-1,3-benzothiazol-7-yl}pyridazin-3-yl) amino]-8-azabicyclo [3.2.1] octane-8-carboxylate (120 mg, C55) as an oil that was taken to the next step without further purification. LCMS (ES, m/z): 628 [M+H]⁺

### Synthesis of Compound 360

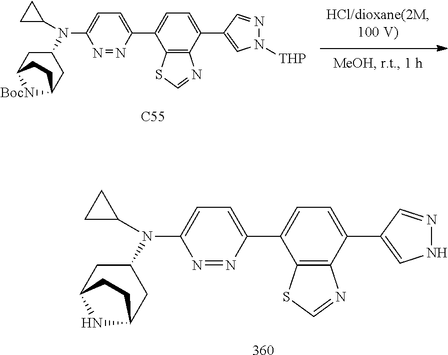

A solution of tert-butyl (exo)-3-[cyclopropyl(6-{4-[1-(oxan-2-yl) pyrazol-4-yl]-1,3-benzothiazol-7-yl} pyridazin-3-yl) amino]-8-azabicyclo [3.2.1] octane-8-carboxylate (120 mg, 0.19 mmol, 1.00 equiv) in HCl(gas)in 1,4-dioxane (0.8 mL) and methanol (2.3 mL) was stirred for 1 hr at room temperature. The resulting mixture was concentrated under vacuum. The crude product was purified by chiral preparative HPLC (Condition 2, Gradient 1) to afford (exo)-N-cyclopropyl-N-{6-[4-(1H-pyrazol-4-yl)-1,3-benzothiazol-7-yl] pyridazin-3-yl}-8-azabicyclo [3.2.1] octan-3-amine) as a solid. LCMS (ES, m/z): 444 [M+H]⁺¹H NMR (400 MHz, DMSO-d₆) δ 13.01 (s, 1H), 9.49 (s, 1H), 8.52 (s, 2H), 8.25 (d, J=9.7 Hz, 1H), 8.09 (d, J=8.0 Hz, 1H), 7.98 (d, J=8.0 Hz, 1H), 7.48 (d, J=9.7 Hz, 1H), 4.90 (tt, J=11.7, 5.4 Hz, 1H), 3.52 (s, 2H), 2.11 (td, J=12.1, 3.0 Hz, 2H), 1.98 (s, 1H), 1.80-1.72 (m, 6H), 1.01 (td, J=6.9, 4.9 Hz, 2H), 0.72-0.64 (m, 2H).

### Example 94: Synthesis of Compound 361

### Synthesis of Compound 361

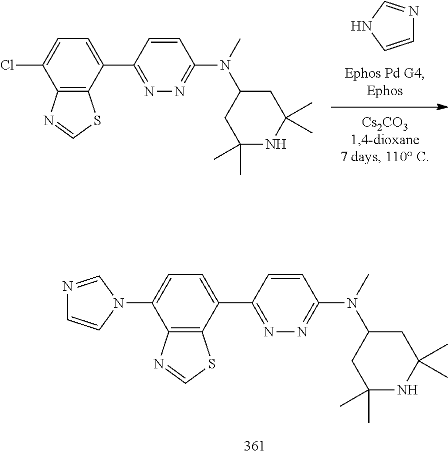 [SMILES: CN(c1ccc(-c2ccc(Cl)c3ncsc23)nn1)C1CC(C)(C)NC(C)(C)C1 ; c1c[nH]cn1 ; CN(c1ccc(-c2ccc(-n3ccnc3)c3ncsc23)nn1)C1CC(C)(C)NC(C)(C)C1]

To a solution of 6-(4-chloro-1,3-benzothiazol-7-yl)-N-methyl-N-(2,2,6,6-tetramethylpiperidin-4-yl) pyridazin-3-amine (70 mg, 0.168 mmol, 1.0 equiv) and imidazole (34.37 mg, 0.504 mmol, 3.0 equiv) in 1,4-dioxane (1.4 mL, 15.864 mmol, 94.43 equiv) and EPhos Pd G4 (15.46 mg, 0.017 mmol, 0.1 equiv) were added EPhos (18. mg, 0.034 mmol, 0.20 equiv) and Cs₂CO₃ (38.44 mg, 0.504 mmol, 3 equiv). After stirring for 7 days at 110° C. under a nitrogen atmosphere, the resulting mixture was concentrated under reduced pressure. The crude product was purified by chiral preparative HPLC (Condition 3, Gradient 1) to afford 6-[4-(imidazol-1-yl)-1,3-benzothiazol-7-yl]-N-methyl-N-(2,2,6,6-tetramethylpiperidin-4-yl)pyridazin-3-amine (1.1 mg, Compound 361) as a solid. LCMS (ES, m/z): 448 [M+H-HCl]+¹H NMR (400 MHz, DMSO-d₆) δ 9.82 (d, J=7.7 Hz, 1H), 9.69 (s, 1H), 9.26 (d, J=17.9 Hz, 1H), 8.50-8.39 (m, 3H), 8.31 (s, 1H), 8.11 (dd, J=8.2, 1.6 Hz, 1H), 7.99 (d, J=4.0 Hz, 1H), 7.48 (dd, J=9.6, 2.2 Hz, 1H), 5.24 (s, 1H), 3.06 (s, 3H), 2.06 (td, J=12.6, 6.0 Hz, 2H), 1.79 (d, J=13.0 Hz, 2H), 1.58 (s, 6H), 1.51 (d, J=2.4 Hz, 6H).

### Example 95: Synthesis of Compound 364

### Synthesis of Intermediate C56

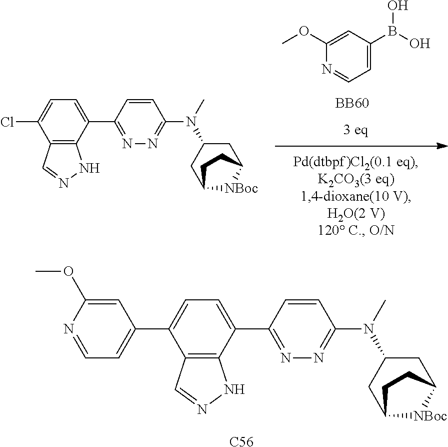

A solution of tert-butyl (exo)-3-{[6-(4-chloro-1H-indazol-7-yl)pyridazin-3-yl](methyl)amino}-8-azabicyclo[3.2.1]octane-8-carboxylate (50 mg, 0.107 mmol, 1.00 equiv), 2-methoxypyridin-4-ylboronic acid (49 mg, 0.321 mmol, 3 equiv), Pd(DtBPF)Cl₂ (7 mg, 0.011 mmol, 0.1 equiv) and K₂CO₃ (44 mg, 0.321 mmol, 3 equiv) in 1,4-dioxane (0.5 mL) and H₂O (0.1 mL) was stirred for overnight at 120° C. under nitrogen atmosphere. The mixture was allowed to cool down to room temperature. The resulting mixture was extracted with EA (3×5 mL), dried over anhydrous Na₂SO₄. After filtration, the filtrate was concentrated under reduced pressure. The residue was purified by silica gel column chromatography, eluted with PE/EA (1:1) to afford tert-butyl (exo)-3-({6-[4-(2-methoxypyridin-4-yl)-1H-indazol-7-yl]pyridazin-3-yl}(methyl)amino)-8-azabicyclo[3.2.1]octane-8-carboxylate (47 mg, C56) as a solid that was taken to the next step without further purification. LCMS (ES, m/z): 542 [M+H]⁺

### Synthesis of Compound 364

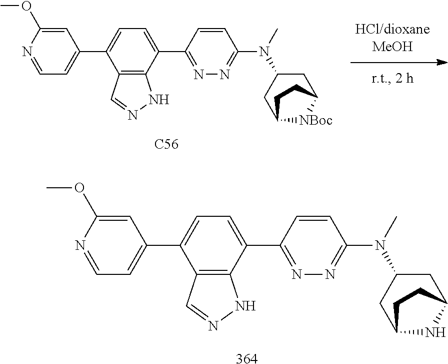

A solution of tert-butyl (exo)-3-({6-[4-(2-methoxypyridin-4-yl)-1H-indazol-7-yl]pyridazin-3-yl}(methyl)amino)-8-azabicyclo[3.2.1]octane-8-carboxylate (47 mg, 0.087 mmol, 1.00 equiv) in MeOH (1 mL) and HCl(gas)in 1,4-dioxane (1 mL) was stirred for 2 h at room temperature. The resulting mixture was concentrated under reduced pressure. The crude product was purified by preparative HPLC (Condition 4, Gradient 13) to afford (exo)-N-{6-[4-(2-methoxypyridin-4-yl)-1H-indazol-7-yl]pyridazin-3-yl}-N-methyl-8-azabicyclo[3.2.1]octan-3-amine (10.1 mg, Compound 364) as a solid. LCMS (ES, m/z): 442 [M+H]⁺¹H NMR (400 MHz, DMSO-d₆) δ 13.30 (s, 1H), 8.37-8.29 (m, 2H), 8.20 (d, J=9.8 Hz, 1H), 8.01 (d, J=7.7 Hz, 1H), 7.48 (d, J=7.6 Hz, 1H), 7.42 (dd, J=5.3, 1.5 Hz, 1H), 7.27 (d, J=9.8 Hz, 1H), 7.18 (d, J=1.6 Hz, 1H), 5.08 (s, 1H), 3.95 (s, 3H), 3.51 (s, 2H), 2.99 (s, 3H), 1.83 (td, J=11.9, 2.9 Hz, 2H), 1.77 (d, J=2.4 Hz, 4H), 1.58 (dt, J=13.3, 4.6 Hz, 2H).

### Example 96: Synthesis of Compound 365

### Synthesis of Intermediate C57

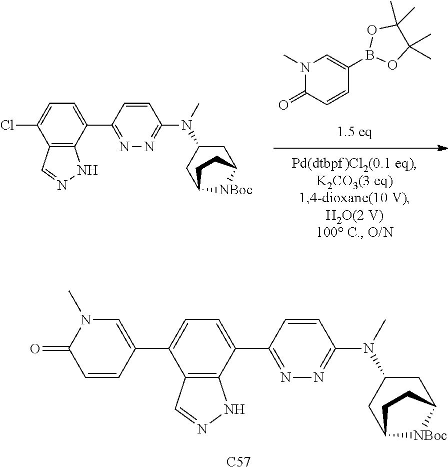

A solution of tert-butyl (exo)-3-{[6-(4-chloro-1H-indazol-7-yl)pyridazin-3-yl](methyl)amino}-8-azabicyclo[3.2.1]octane-8-carboxylate (60 mg, 0.128 mmol, 1.00 equiv) and 1-methyl-5-(4,4,5,5-tetramethyl-1,3,2-dioxaborolan-2-yl)pyridin-2-one (45 mg, 0.192 mmol, 1.5 equiv), Pd(DtBPF)Cl₂ (8 mg, 0.013 mmol, 0.1 equiv) and K₂CO₃ (53 mg, 0.384 mmol, 3 equiv) in 1,4-dioxane (0.6 mL) and H₂O (0.15 mL) was stirred for overnight at 100° C. under nitrogen atmosphere. The mixture was allowed to cool down to room temperature. The resulting mixture was extracted with ethyl acetate (3×5 mL), dried over anhydrous Na₂SO₄. After filtration, the filtrate was concentrated under reduced pressure. The residue was purified by silica gel column chromatography, eluted with PE/EA (1:1) to afford tert-butyl (exo)-3-[methyl({6-[4-(1-methyl-6-oxopyridin-3-yl)-1H-indazol-7-yl]pyridazin-3-yl})amino]-8-azabicyclo[3.2.1]octane-8-carboxylate (63 mg, C57) as a solid that was taken to the next step without further purification. LCMS (ES, m/z): 542 [M+H]⁺

### Synthesis of Compound 365

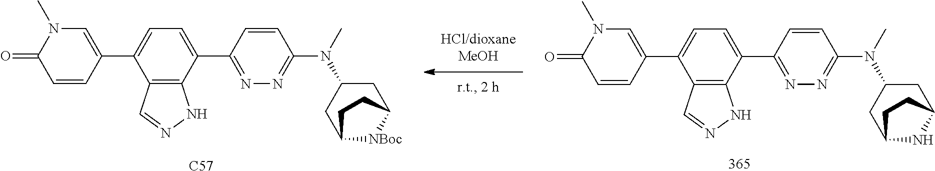

A solution of tert-butyl (exo)-3-[methyl({6-[4-(1-methyl-6-oxopyridin-3-yl)-1H-indazol-7-yl]pyridazin-3-yl})amino]-8-azabicyclo[3.2.1]octane-8-carboxylate (63 mg, 0.116 mmol, 1.00 equiv) in MeOH (1 mL), HCl(gas)in 1,4-dioxane (1 mL) was stirred for 2 hr at room temperature. The resulting mixture was concentrated under reduced pressure. The crude product was purified by preparative HPLC (Condition 7, Gradient 2) to afford 5-(7-{6-[(exo)-8-azabicyclo[3.2.1]octan-3-yl(methyl)amino]pyridazin-3-yl}-1H-indazol-4-yl)-1-methylpyridin-2-one (10.4 mg, Compound 365) as a solid. LCMS (ES, m/z): 442 [M+H] ¹H NMR (400 MHz, DMSO-d₆) δ 13.18 (s, 1H), 8.37 (s, 1H), 8.22 (d, J=2.7 Hz, 1H), 8.17 (d, J=9.8 Hz, 1H), 7.98-7.89 (m, 2H), 7.29 (d, J=15.4, 8.7 Hz, 1H), 7.26 (d, 1H) 6.58 (d, J=9.4 Hz, 1H), 5.10-5.03 (m, 1H), 3.59 (s, 3H), 3.51 (s, 2H), 2.98 (s, 3H), 2.17 (s, 1H), 1.82 (dd, 2H), 1.76 (s, 4H) 1.61-1.52 (m, 2H).

### Example 97: Synthesis of Compound 369

### Synthesis of Intermediate C58

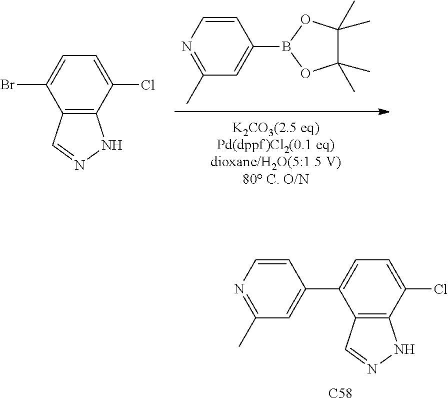

A mixture of 4-bromo-7-chloro-1H-indazole (200.00 mg, 0.8 mmol, 1.00 equiv), 2-methyl-4-(4,4,5,5-tetramethyl-1,3,2-dioxaborolan-2-yl) pyridine (189.29 mg, 0.8 mmol, 1.00 equiv), Pd(dppf)Cl₂ (63.22 mg, 0.08 mmol, 0.10 equiv) and K₂CO₃ (298.53 mg, 2.1 mmol, 2.50 equiv) in dioxane (5.0 mL) and H₂O (1.0 mL) was stirred overnight at 80° C. under nitrogen atmosphere. The mixture was allowed to cool down to 25° C. The resulting mixture was diluted with water (50.0 mL). The resulting mixture was extracted with EA (2×50.0 mL), dried over anhydrous Na₂SO₄. After filtration, the filtrate was concentrated under reduced pressure. The residue was purified by silica gel column chromatography, eluted with PE/EA (1:2) to afford 7-chloro-4-(2-methylpyridin-4-yl)-1H-indazole (220.00 mg, C58) as a solid that was taken to the next step without further purification. LCMS (ES, m/z): 244 [M+H]⁺

### Synthesis of Intermediate C59

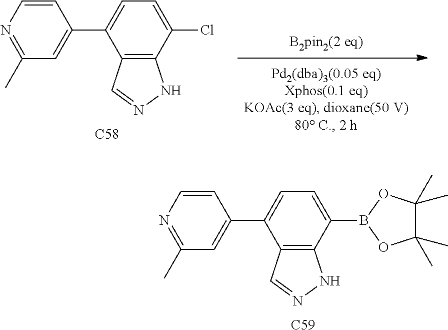 [SMILES: Cc1cc(-c2ccc(Cl)c3[nH]ncc23)ccn1 ; "C58" ; Cc1cc(-c2ccc(B3OC(C)(C)C(C)(C)O3)c3[nH]ncc23)ccn1 ; "C59"]

A mixture of 7-chloro-4-(2-methylpyridin-4-yl)-1H-indazole (230.00 mg, 0.9 mmol, 0.62 equiv), bis(pinacolato)diboron (383.48 mg, 1.5 mmol, 2.00 equiv), Pd₂(dba)₃ (34.57 mg, 0.03 mmol, 0.05 equiv), XPhos (35.99 mg, 0.07 mmol, 0.10 equiv) and potassium acetate (222.31 mg, 2.2 mmol, 3.00 equiv) in dioxane (11.5 mL) was stirred for 2 hr at 80° C. under nitrogen atmosphere. The mixture was allowed to cool down to 25° C. The resulting mixture was diluted with water (50.0 mL). The resulting mixture was extracted with ethyl acetate (2×50.0 mL), dried over anhydrous Na₂SO₄. After filtration, the filtrate was concentrated under reduced pressure to afford crude 4-(2-methylpyridin-4-yl)-7-(4,4,5,5-tetramethyl-1,3,2-dioxaborolan-2-yl)-1H-indazole (602.00 mg, C59) as a semi-solid that was taken to the next step without further purification. LCMS (ES, m/z): 336 [M+H]⁺

### Synthesis of Intermediate C60

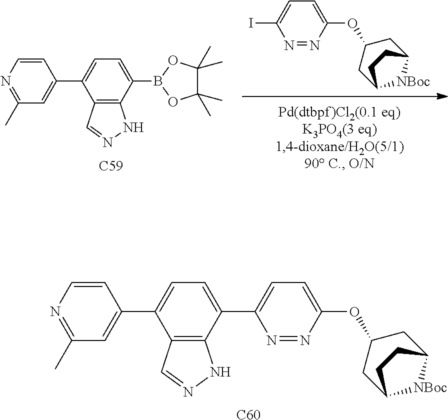

A mixture of 4-(2-methylpyridin-4-yl)-7-(4,4,5,5-tetramethyl-1,3,2-dioxaborolan-2-yl)-1H-indazole (65 mg, 0.194 mmol, 1.39 equiv), tert-butyl (exo)-3-[(6-iodopyridazin-3-yl) oxy]-8-azabicyclo [3.2.1] octane-8-carboxylate (60 mg, 0.139 mmol, 1.00 equiv), Pd(DtBPF)Cl₂ (9.07 mg, 0.014 mmol, 0.1 equiv) and K₃PO₄ (88.59 mg, 0.417 mmol, 3 equiv) in dioxane (1.0 mL) and H₂O (0.2 mL) was stirred for 16 hr at 90° C. under nitrogen atmosphere. The mixture was allowed to cool down to 25° C. The resulting mixture was diluted with water (50.0 mL). The resulting mixture was extracted with EA (2×50.0 mL), dried over anhydrous Na₂SO₄. After filtration, the filtrate was concentrated under reduced pressure. The residue was purified by silica gel column chromatography, eluted with PE/EA (1:5) to afford tert-butyl (exo)-3-({6-[4-(2-methylpyridin-4-yl)-1H-indazol-7-yl] pyridazin-3-yl} oxy)-8-azabicyclo [3.2.1] octane-8-carboxylate (63.00 mg, C60) as a solid that was taken to the next step without further purification. LCMS (ES, m/z): 513 [M+H]⁺

### Synthesis of Compound 369

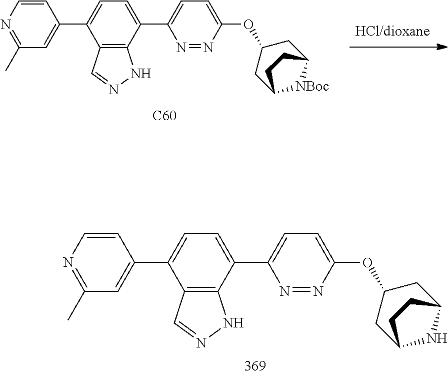

A solution of tert-butyl (exo)-3-({6-[4-(2-methylpyridin-4-yl)-1H-indazol-7-yl] pyridazin-3-yl} oxy)-8-azabicyclo [3.2.1] octane-8-carboxylate (40.00 mg, 0.07 mmol, 1.00 equiv) in HCl(gas)in 1,4-dioxane (0.4 mL) and methanol (1.2 mL) was stirred for 1 hr at room temperature. The resulting mixture was concentrated under vacuum. The crude product was purified by preparative chiral HPLC (Condition 3, Gradient 3) to afford 7-{6-[(exo)-8-azabicyclo [3.2.1] octan-3-yloxy] pyridazin-3-yl}-4-(2-methylpyridin-4-yl)-1H-indazole (2.00 mg, Compound 369) as a solid. LCMS (ES, m/z): 413 [M+H]⁺¹H NMR (400 MHz, DMSO-d₆) δ 13.57 (s, 1H), 9.09 (d, J=32.1 Hz, 2H), 8.88 (d, J=6.1 Hz, 1H), 8.55 (d, J=10.6 Hz, 2H), 8.32 (s, 1H), 8.24 (d, J=7.6 Hz, 2H), 7.75 (d, J=7.7 Hz, 1H), 7.48 (d, J=9.3 Hz, 1H), 5.69 (dq, J=11.0, 5.5 Hz, 1H), 4.17 (s, 2H), 3.76 (s, 2H), 2.86-2.81 (m, 3H), 2.05 (d, J=11.2 Hz, 6H).

### Example 98: Synthesis of Compound 370

### Synthesis of Intermediate C61

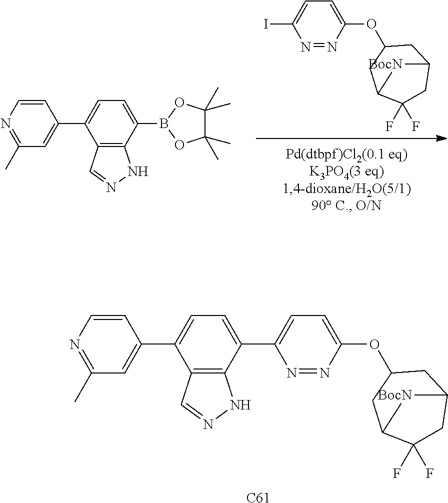 [SMILES: Cc1cc(-c2ccc(B3OC(C)(C)C(C)(C)O3)c3[nH]ncc23)ccn1 ; CC(C)(C)OC(=O)N1C2CC(Oc3ccc(I)nn3)CC1C(F)(F)C2 ; Cc1cc(-c2ccc(-c3ccc(OC4CC5CC(F)(F)C(C4)N5C(=O)OC(C)(C)C)nn3)c3[nH]ncc23)ccn1 ; "C61"]

A mixture of tert-butyl 6,6-difluoro-3-[(6-iodopyridazin-3-yl) oxy]-8-azabicyclo [3.2.1]octane-8-carboxylate (60.00 mg, 0.12 mmol, 1.00 equiv), 4-(2-methylpyridin-4-yl)-7-(4,4,5,5-tetramethyl-1,3,2-dioxaborolan-2-yl)-1H-indazole (60.32 mg, 0.180 mmol, 1.40 eq.), Pd(DtBPF)Cl₂ (8.20 mg, 0.01 mmol, 0.10 eq.) and tripotassium phosphate (81.77 mg, 0.3 mmol, 3.00 eq.) in dioxane (1.0 mL) and water (0.2 mL) was stirred for 16 hr at 90° C. under a nitrogen atmosphere. The mixture was allowed to cool to 25° C. The resulting mixture was diluted with water (50.0 mL). The resulting mixture was extracted with ethyl acetate (2×50.0 mL), dried over anhydrous sodium sulfate. After filtration, the filtrate was concentrated under reduced pressure. The residue was purified by silica gel column chromatography, eluted with petroleum ether/ethyl acetate (1:5) to afford tert-butyl 6,6-difluoro-3-({6-[4-(2-methylpyridin-4-yl)-1H-indazol-7-yl] pyridazin-3-yl} oxy)-8-azabicyclo [3.2.1] octane-8-carboxylate (48.00 mg, C61) as a solid that was taken to the next step without further purification. LCMS (ES, m/z): 549 [M+H]

### Synthesis of Compound 370

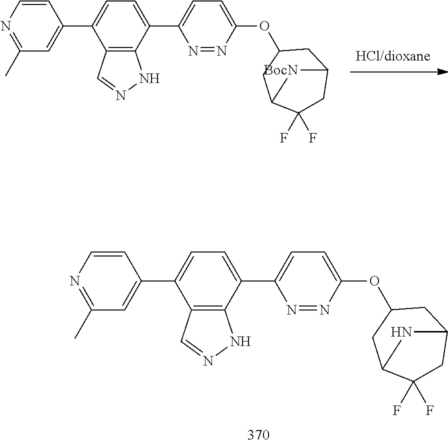 [SMILES: Cc1cc(-c2ccc(-c3ccc(OC4CC5CC(F)(F)C(C4)N5C(=O)OC(C)(C)C)nn3)c3[nH]ncc23)ccn1 ; Cc1cc(-c2ccc(-c3ccc(OC4CC5CC(F)(F)C(C4)N5)nn3)c3[nH]ncc23)ccn1 ; "370"]

A solution of tert-butyl 6,6-difluoro-3-({6-[4-(2-methylpyridin-4-yl)-1H-indazol-7-yl]pyridazin-3-yl} oxy)-8-azabicyclo [3.2.1] octane-8-carboxylate (40.00 mg, 0.07 mmol, 1.00 equiv) in hydrochloric acid(gas)in 1,4-dioxane (0.4 mL) and methanol (1.2 mL) was stirred for 1 hr at room temperature. The resulting mixture was concentrated under vacuum. The crude product was purified by preparative chiral HPLC (Condition 3, Gradient 1) to afford 7-(6-{6,6-difluoro-8-azabicyclo [3.2.1] octan-3-yl] oxy} pyridazin-3-yl)-4-(2-methylpyridin-4-yl)-1H-indazole (8.60 mg, Compound 370) as a solid. LCMS (ES, m/z): 449 [M+H] ⁺¹H NMR (400 MHz, DMSO-d₆) δ 13.60 (s, 1H), 10.62 (s, 1H), 10.19 (s, 1H), 8.90 (d, J 6.2 Hz, 1H), 8.61-8.53 (m, 2H), 8.39 (s, 1H), 8.31 (dd, J 6.2, 1.9 Hz, 1H), 8.24 (d, J 7.8 Hz, 1H), 7.77 (d, J 7.6 Hz, 1H), 7.51 (d, J 9.4 Hz, 1H), 5.77 (tt, J 11.1, 6.2 Hz, 1H), 4.51 (d, J 12.2 Hz, 1H), 4.39 (s, 1H), 2.87 (s, 3H), 2.83-2.70 (m, 3H), 2.57 (s, 1H), 2.39 (d, J 11.9 Hz, 1H), 2.26 (t, J 12.7 Hz, 1H).

### Example 99: Synthesis of Compound 371

### Synthesis of Intermediate C62

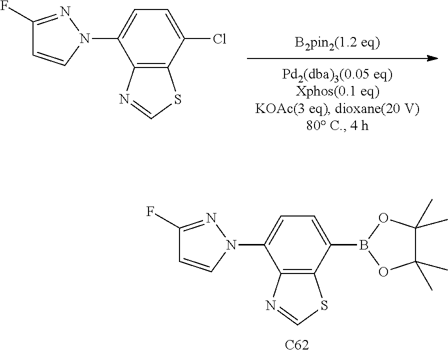 [SMILES: Fc1ccn(-c2ccc(Cl)c3scnc23)n1 ; CC1(C)OB(c2ccc(-n3ccc(F)n3)c3ncsc23)OC1(C)C ; "C62"]

A mixture of 7-chloro-4-(3-fluoropyrazol-1-yl)-1,3-benzothiazole (70.00 mg, 0.2 mmol, 1.00 eq.), bis(pinacolato)diboron (84.09 mg, 0.3 mmol, 1.20 eq.), Pd₂(dba)₃ (14.28 mg, 0.01 mmol, 0.05 eq.), XPhos (13.15 mg, 0.02 mmol, 0.10 eq.) and potassium acetate (81.24 mg, 0.8 mmol, 3.00 equiv) in dioxane (4.0 mL) was stirred overnight at 80° C. under a nitrogen atmosphere. The mixture was allowed to cool down to 25° C. The resulting mixture was diluted with water (50.0 mL). The resulting mixture was extracted with ethyl acetate (2×50.0 mL), dried over anhydrous sodium sulfate. After filtration, the filtrate was concentrated under reduced pressure to afford crude 4-(3-fluoropyrazol-1-yl)-7-(4,4,5,5-tetramethyl-1,3,2-dioxaborolan-2-yl)-1,3-benzothiazole (150.00 mg, C62) as an oil that was taken to the next step without further purification. LCMS (ES, m/z): 346 [M+H]⁺

### Synthesis of Intermediate C63

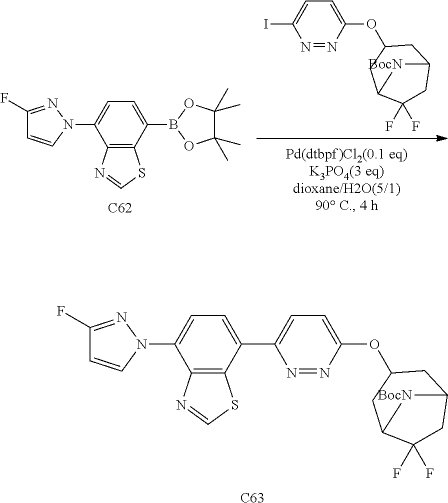 [SMILES: CC1(C)OB(c2ccc(-n3ccc(F)n3)c3ncsc23)OC1(C)C ; CC(C)(C)OC(=O)N1C2CC(Oc3ccc(I)nn3)CC1C(F)(F)C2 ; "C62" ; CC(C)(C)OC(=O)N1C2CC(Oc3ccc(-c4ccc(-n5ccc(F)n5)c5ncsc45)nn3)CC1C(F)(F)C2 ; "C63"]

A mixture of 4-(3-fluoropyrazol-1-yl)-7-(4,4,5,5-tetramethyl-1,3,2-dioxaborolan-2-yl)-1,3-benzothiazole (150.00 mg, 0.4 mmol, 2.71 eq.), tert-butyl 6,6-difluoro-3-[(6-iodopyridazin-3-yl) oxy]-8-azabicyclo [3.2.1] octane-8-carboxylate (75.00 mg, 0.1 mmol, 1.00 eq.), Pd(DtBPF)Cl₂ (10.46 mg, 0.01 mmol, 0.10 eq.) and tripotassium phosphate (102.21 mg, 0.4 mmol, 3.00 eq.) in dioxane (6.0 mL) and water (1.2 mL) was stirred for 16 hr at 90° C. under a nitrogen atmosphere. The mixture was allowed to cool to 25° C. The resulting mixture was diluted with water (50.0 mL). The resulting mixture was extracted with ethyl acetate (2×50.0 mL), dried over anhydrous sodium sulfate. After filtration, the filtrate was concentrated under reduced pressure. The residue was purified by silica gel column chromatography, eluted with petroleum ether/ethyl acetate (1:1) to afford tert-butyl 6,6-difluoro-3-({6-[4-(3-fluoropyrazol-1-yl)-1,3-benzothiazol-7-yl]pyridazin-3-yl} oxy)-8-azabicyclo [3.2.1] octane-8-carboxylate (60.00 mg, C63) as a solid. LCMS (ES, m/z): 559 [M+H]⁺

### Synthesis of Compound 371

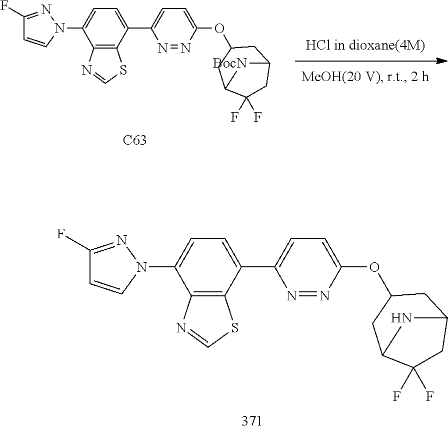 [SMILES: CC(C)(C)OC(=O)N1C2CC(Oc3ccc(-c4ccc(-n5ccc(F)n5)c5ncsc45)nn3)CC1C(F)(F)C2 ; "C63" ; Fc1ccn(-c2ccc(-c3ccc(OC4CC5CC(F)(F)C(C4)N5)nn3)c3scnc23)n1 ; "371"]

A solution of tert-butyl 6,6-difluoro-3-({6-[4-(3-fluoropyrazol-1-yl)-1,3-benzothiazol-7-yl]pyridazin-3-yl} oxy)-8-azabicyclo [3.2.1] octane-8-carboxylate (30.00 mg, 0.05 mmol, 1.00 eq.) in hydrochloric acid(gas)in 1,4-dioxane (0.5 mL) and methanol (1.5 mL) was stirred for 1 hr at room temperature. The resulting mixture was concentrated under vacuum. The crude product was purified by preparative chiral HPLC (Condition 2, Gradient 1) to afford 7-[6-({6,6-difluoro-8-azabicyclo [3.2.1] octan-3-yl} oxy) pyridazin-3-yl]-4-(3-fluoropyrazol-1-yl)-1,3-benzothiazole (1.80 mg, Compound 371) as a solid. LCMS (ES, m/z): 459 [M+H]⁺¹H NMR (400 MHz, DMSO-d₆) δ 9.66 (s, 1H), 9.14 (t, J=2.7 Hz, 1H), 8.56 (d, J=9.5 Hz, 1H), 8.40 (d, J=8.4 Hz, 1H), 8.09 (d, J=8.4 Hz, 1H), 7.41 (d, J=9.4 Hz, 1H), 6.46 (dd, J=5.7, 2.8 Hz, 1H), 5.75 (td, J=10.7, 5.4 Hz, 1H), 3.67 (d, J=7.2 Hz, 1H), 3.52 (d, J=13.1 Hz, 1H), 2.98 (s, 1H), 2.36-2.32 (m, 3H), 1.74 (dt, J=34.1, 11.1 Hz, 2H).

### Example 101: Synthesis of Compound 373

### Synthesis of Intermediate C67

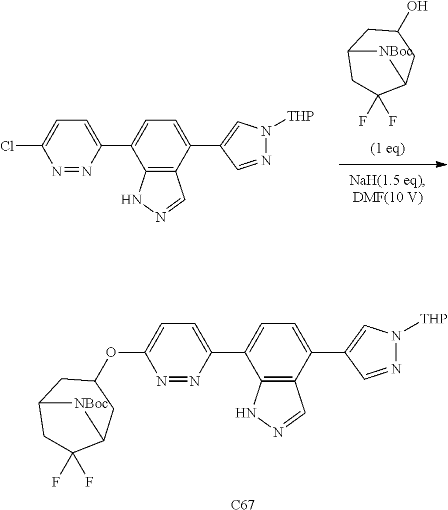

To a solution of tert-butyl 6,6-difluoro-3-hydroxy-8-azabicyclo[3.2.1]octane-8-carboxylate (55 mg, 0.210 mmol, 1 equiv) in DMF was added sodium hydride (60% in oil, 3 mg) at 0° C. The mixture was stirred for 15 min. 7-(6-chloropyridazin-3-yl)-4-[1-(oxan-2-yl)pyrazol-4-yl]-1H-indazole (80 mg, 0.210 mmol, 1.00 equiv) was added and the mixture was allowed to warm to RT and stirred for 2 h. The reaction mixture was quenched by water and extracted with DCM (3×25 mL). The resulting mixture was concentrated under reduced pressure. This resulted in tert-butyl 6,6-difluoro-3-[(6-{4-[1-(oxan-2-yl)pyrazol-4-yl]-1H-indazol-7-yl}pyridazin-3-yl)oxy]-8-azabicyclo[3.2.1]octane-8-carboxylate (80 mg, C67) as a solid that was taken to the next step without further purification. LCMS (ES, m/z): 608 [M+H]⁺

### Synthesis of Compound 373

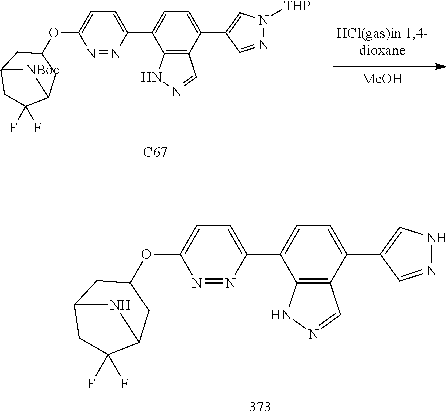

Into a 50 mL round-bottom flask were added tert-butyl 6,6-difluoro-3-[(6-{4-[1-(oxan-2-yl)pyrazol-4-yl]-1H-indazol-7-yl}pyridazin-3-yl)oxy]-8-azabicyclo[3.2.1]octane-8-carboxylate (80 mg, 0.132 mmol, 1.00 equiv) and HCl(gas)in 1,4-dioxane (2 mL), MeOH (2 mL) at room temperature. The resulting mixture was stirred for 30 min at room temperature under nitrogen atmosphere. The resulting mixture was concentrated under reduced pressure. The residue was dissolved in DMF (5 mL). The residue was purified by reverse flash chromatography with the following conditions: Column: YMC-Actus Triart C18, 30×150 mm, 5 μm; Mobile Phase A: water (0.05% HCl), Mobile Phase B: ACN; Flow rate: 60 mL/min; Gradient: 5% B to 33% B in 8 min, 33% B; Wave Length: 220 nm; RT1(min):7.03) to afford 7-[6-({6,6-difluoro-8-azabicyclo[3.2.1]octan-3-yl}oxy)pyridazin-3-yl]-4-(1H-pyrazol-4-yl)-1H-indazole (5.8 mg, Compound 373) as a solid. LCMS (ES, m/z): 424 [M+H]⁺¹H NMR (400 MHz, DMSO-d6) δ 13.22 (s, 2H), 8.55 (s, 1H), 8.53 (s, 1H), 8.42 (d, J=9.4 Hz, 1H), 8.22 (s, 1H), 8.00 (d, J=7.8 Hz, 1H), 7.51 (d, J=7.6 Hz, 1H), 7.34 (d, J=9.3 Hz, 1H), 5.75 (tt, J=11.1, 6.4 Hz, 1H), 3.67 (d, J=7.5 Hz, 1H), 3.52 (d, J=13.1 Hz, 1H), 2.98 (s, 1H), 2.45-2.40 (m, 1H), 2.38-2.33 (m, 1H), 2.24 (q, J=14.1, 13.6 Hz, 1H), 1.76-1.67 (m, 1H)⁻

### Example 102: Synthesis of Compound 374

### Synthesis of Intermediate C68

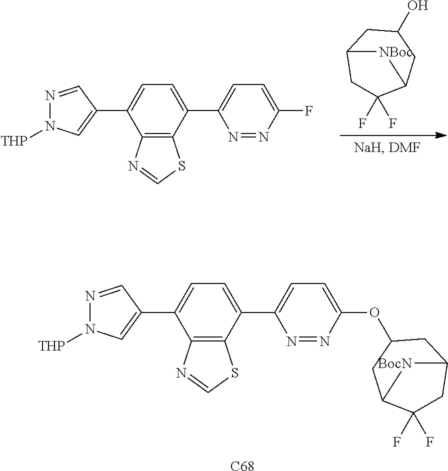

To a solution of tert-butyl 6,6-difluoro-3-hydroxy-8-azabicyclo[3.2.1]octane-8-carboxylate (55 mg, 0.210 mmol, 1 eq.) in dimethylformamide was added sodium hydride (60% in oil, 3 mg) at 0° C. The mixture was stirred for 15 min. 7-(6-chloropyridazin-3-yl)-4-[1-(oxan-2-yl)pyrazol-4-yl]-1H-indazole (80 mg, 0.210 mmol, 1.00 eq.) was added and the mixture was allowed to warm to room temperature and stirred for 2 hr. The reaction mixture was quenched by water and extracted with dichloromethane (3×25 mL). The resulting mixture was concentrated under reduced pressure. This resulted in tert-butyl 6,6-difluoro-3-[(6-{4-[1-(oxan-2-yl)pyrazol-4-yl]-1H-indazol-7-yl}pyridazin-3-yl)oxy]-8-azabicyclo[3.2.1]octane-8-carboxylate (80 mg, C68) as a solid that was taken to the next step without further purification. LCMS (ES, m/z): 608 [M+H]⁺

### Synthesis of Compound 374

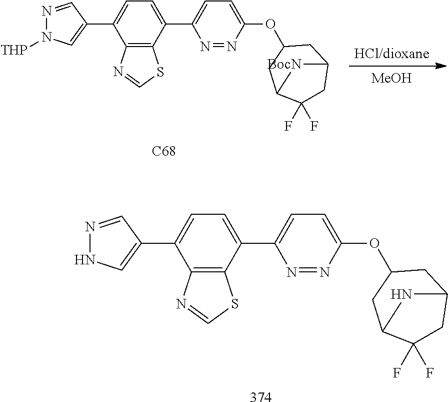

To a 50 mL round-bottom flask were added tert-butyl 6,6-difluoro-3-[(6-{4-[1-(oxan-2-yl)pyrazol-4-yl]-1H-indazol-7-yl}pyridazin-3-yl)oxy]-8-azabicyclo[3.2.1]octane-8-carboxylate (80 mg, 0.132 mmol, 1.00 eq.) and hydrochloric acid (gas) in 1,4-dioxane (2 mL), methanol (2 mL) at room temperature. The resulting mixture was stirred for 30 min at room temperature under a nitrogen atmosphere. The resulting mixture was concentrated under reduced pressure. The residue was dissolved in dimethylformamide (5 mL). The residue was purified by preparative HPLC (Condition 7, Gradient 5) to afford 7-[6-({6,6-difluoro-8-azabicyclo[3.2.1]octan-3-yl}oxy)pyridazin-3-yl]-4-(1H-pyrazol-4-yl)-1H-indazole (5.8 mg, Compound 374) as a solid. ¹H NMR (400 MHz, DMSO-d6) δ 13.22 (s, 2H), 8.55 (s, 1H), 8.53 (s, 1H), 8.42 (d, J=9.4 Hz, 1H), 8.22 (s, 1H), 8.00 (d, J=7.8 Hz, 1H), 7.51 (d, J=7.6 Hz, 1H), 7.34 (d, J=9.3 Hz, 1H), 5.75 (tt, J=11.1, 6.4 Hz, 1H), 3.67 (d, J=7.5 Hz, 1H), 3.52 (d, J=13.1 Hz, 1H), 2.98 (s, 1H), 2.45-2.40 (m, 1H), 2.38-2.33 (m, 1H), 2.24 (q, J=14.1, 13.6 Hz, 1H), 1.76-1.67 (m, 1H)⁻

### Example 103: Synthesis of Compound 375

### Synthesis of Intermediate C69

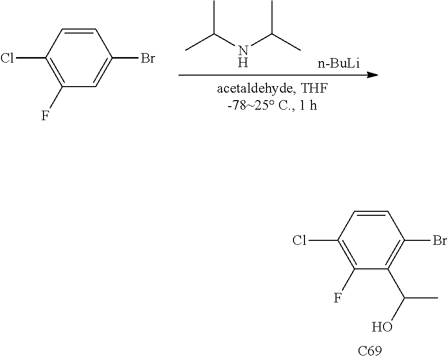

To a stirred solution of diisopropylamine (19.29 g, 190.986 mmol, 2.0 equiv) in THE (266 mL) was added n-BuLi (76.4 mL, 190.986 mmol, 2.0 equiv) (2.5 M) dropwise at −30° C. under N₂ atmosphere. The resulting mixture was stirred for 30 min at −30° C. To the above mixture was added 4-bromo-1-chloro-2-fluorobenzene (20.00 g, 95.493 mmol, 1.0 equiv) in THE (50 mL) dropwise at −30° C. under N₂ atmosphere. The resulting mixture was stirred for additional 30 min at −30° C. under N₂ atmosphere. Followed by the addition of acetaldehyde (12.6 g, 286.479 mmol, 3.0 equiv), the resulting mixture was stirred for additional 1 hr at −30° C. The reaction was quenched with saturated aqueous NH₄Cl. The resulting mixture was extracted with EA (2×400 mL). The combined organic layers were washed with water (700 mL) and brine (700 mL), dried over anhydrous Na₂SO₄. After filtration, the filtrate was concentrated under reduced pressure. The residue was purified by silica gel column chromatography, eluted with PE/EA (4/1) to afford 1-(6-bromo-3-chloro-2-fluorophenyl)ethanol (13.0 g, C69) as an oil that was taken to the next step without further purification.

### Synthesis of Intermediate C70

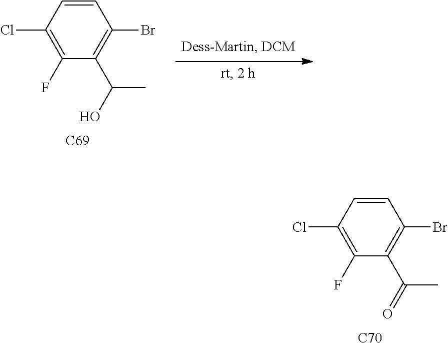

To a stirred mixture of 1-(6-bromo-3-chloro-2-fluorophenyl)ethanol (13.0 g, 51.282 mmol, 1.0 equiv) in DCM (260 mL) was added Dess-Martin periodinane (32.6 g, 76.923 mmol, 1.5 equiv) in portions at room temperature. The resulting mixture was stirred for 2 hr at room temperature. The resulting mixture was diluted with water (300 mL) and extracted with EA (300 mL). The combined organic layers were washed with Na₂SO₃ (aq.) (2×300 mL), water (300 mL) and brine (300 mL), dried over anhydrous Na₂SO₄. After filtration, the filtrate was concentrated under reduced pressure. The residue was purified by silica gel column chromatography, eluted with PE/EA (4/1) to afford 1-(6-bromo-3-chloro-2-fluorophenyl)ethanone (11.6 g, C70) as a solid that was taken to the next step without further purification. LCMS (ES, m/z): 249.2[M−H]⁻

### Synthesis of Intermediate C71

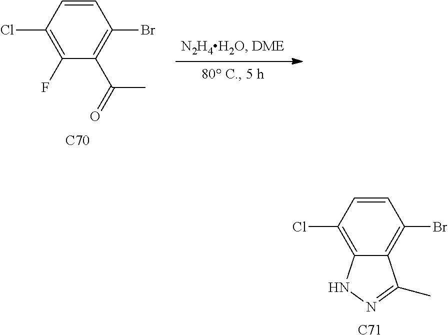

To a stirred solution of 1-(6-bromo-3-chloro-2-fluorophenyl)ethanone (11.6 g, 46.127 mmol, 1.0 equiv) in DME (100 mL) was added N₂H₄·H₂O (50 mL) at room temperature. The resulting mixture was stirred for 2 hr at 80° C. The resulting mixture was diluted with water (400 mL) and extracted with EA (2×400 mL). The combined organic layers were washed with water (600 mL) and brine (600 mL), dried over anhydrous Na₂SO₄. After filtration, the filtrate was concentrated under reduced pressure. The residue was purified by silica gel column chromatography, eluted with PE/EA(2/1) to afford 4-bromo-7-chloro-3-methyl-1H-indazole (6.8 g, C71) as a solid that was taken to the next step without further purification. LCMS (ES, m/z): 245.0 [M+H]⁺

### Synthesis of Intermediate C72

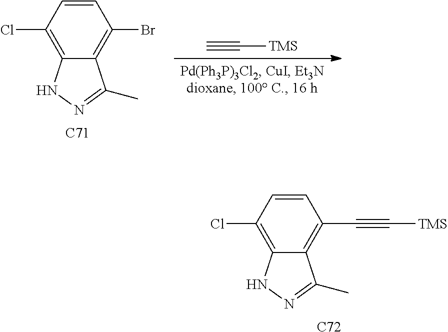

To a stirred solution of 4-bromo-7-chloro-3-methyl-1H-indazole (6.80 g, 27.699 mmol, 1.0 equiv) and trimethylsilylacetylene (4.10 g, 41.549 mmol, 1.5 equiv) in dioxane (80 mL) was added Et₃N (8.40 g, 83.097 mmol, 3.0 equiv) and CuI (0.53 g, 2.770 mmol, 0.1 equiv) and Pd(PPh₃)₂Cl₂ (1.94 g, 2.770 mmol, 0.1 equiv) at rt under N₂ atmosphere. The resulting mixture was stirred for 16 h at 100° C. under N₂ atmosphere. The resulting mixture was diluted with water (200 mL) and extracted with EA (2×200 mL). The combined organic layers were washed with water (400 mL) and brine (400 mL), dried over anhydrous Na₂SO₄. After filtration, the filtrate was concentrated under reduced pressure. The residue was purified by silica gel column chromatography, eluted with PE/EA (2/1) to afford 7-chloro-3-methyl-4-[2-(trimethylsilyl)ethynyl]-1H-indazole (7.2 g, C72) as a solid that was taken to the next step without further purification. LCMS (ES, m/z): 263.1 [M+H]⁺

### Synthesis of Intermediate C73

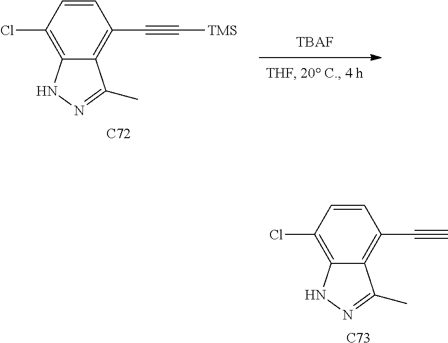 [SMILES: Cc1n[nH]c2c(Cl)ccc(C#C[Si](C)(C)C)c12 ; "TBAF" ; "C72" ; C#Cc1ccc(Cl)c2[nH]nc(C)c12 ; "C73"]

To a stirred solution of 7-chloro-3-methyl-4-[2-(trimethylsilyl)ethynyl]-1H-indazole (7.2 g, 27.396 mmol, 1.0 equiv) in THF (72 mL) was added TBAF (1.43 g, 5.479 mmol, 0.2 equiv) at room temperature. The resulting mixture was stirred for 4 hr at room temperature. The resulting mixture was diluted with water (200 mL) and extracted with EA (2×200 mL). The combined organic layers were washed with water (400 mL) and brine (400 mL), dried over anhydrous Na₂SO₄. After filtration, the filtrate was concentrated under reduced pressure. The residue was purified by silica gel column chromatography, eluted with PE/EA (2/1) to afford 7-chloro-4-ethynyl-3-methyl-1H-indazole (4.3 g, C73) as a solid that was taken to the next step without further purification. LCMS (ES, m/z): 191.1 [M+H]⁺

### Synthesis of Intermediate C74

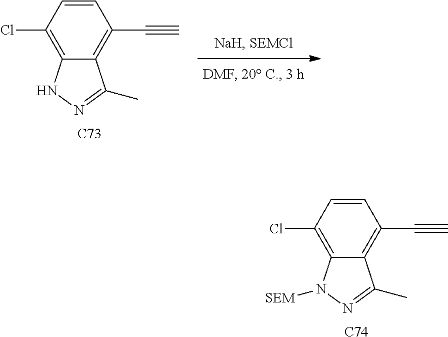

To a solution of 7-chloro-4-ethynyl-3-methyl-1H-indazole (4.2 g, 22.032 mmol, 1.0 equiv) in DMF (80 mL) was added NaH (1.32 g, 33.048 mmol, 1.5 equiv) at 0° C. The mixture was stirred for 10 min. SEMCl (5.51 g, 33.048 mmol, 1.5 equiv) was added and the mixture was allowed to warm to room temperature and stirred for 3 hr. The reaction mixture was quenched by NH₄Cl. The resulting mixture was diluted with water (200 mL) and extracted with EA (2×200 mL). The combined organic layers were washed with water (2×300 mL), brine (200 mL) and dried over anhydrous Na₂SO₄. After filtration, the filtrate was concentrated under reduced pressure. The residue was purified by silica gel column chromatography, eluted with PE/EA (4:1) to afford 7-chloro-4-ethynyl-3-methyl-1-{[2-(trimethylsilyl)ethoxy]methyl}indazole (1.25 g, C74) as a solid that was taken to the next step without further purification. LCMS (ES, m/z): 321.2 [M+H]⁺

### Synthesis of Intermediate C75

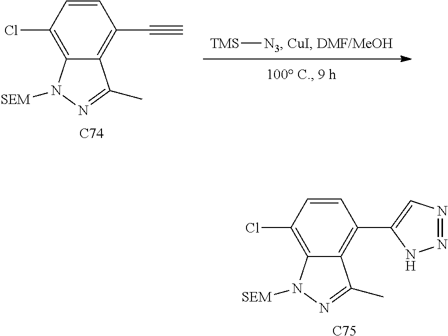 [SMILES: C#Cc1ccc(Cl)c2c1c(C)nn2COCC[Si](C)(C)C ; C[Si](C)(C)N=[N+]=[N-] ; "CuI, DMF/MeOH" ; "C74" ; Cc1nn(COCC[Si](C)(C)C)c2c(Cl)ccc(-c3cnn[nH]3)c12 ; "C75"]

To a stirred mixture of 7-chloro-4-ethynyl-3-methyl-1-{[2-(trimethylsilyl)ethoxy]methyl}indazole (1.15 g, 3.584 mmol, 1.0 equiv) in DMF (26.0 mL) and MeOH (2.8 mL) were added CuI (68.3 mg, 0.358 mmol, 0.1 equiv) and azidotrimethylsilane (0.83 g, 7.168 mmol, 2.0 equiv) at room temperature under N₂ atmosphere. The resulting mixture was stirred for 9 hr at 100° C. under N₂ atmosphere. The resulting mixture was diluted with water (100 mL) and extracted with EA (2×100 mL). The combined organic layers were washed with water (150 mL) and brine (150 mL), dried over anhydrous Na₂SO₄. After filtration, the filtrate was concentrated under reduced pressure. The residue was purified by silica gel column chromatography, eluted with PE/EA (2:1) to afford 7-chloro-3-methyl-4-(3H-1,2,3-triazol-4-yl)-1-{[2-(trimethylsilyl)ethoxy]methyl}indazole (940.0 mg, C75) as an oil that was taken to the next step without further purification. LCMS (ES, m/z): 364.1[M+H]⁺

### Synthesis of Intermediate C76

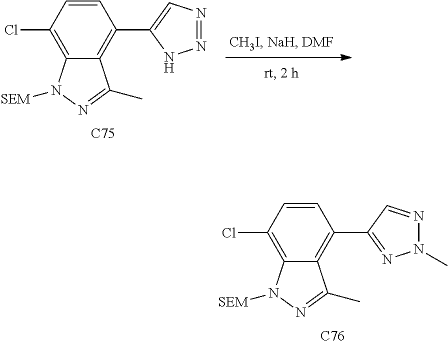

To a solution of 7-chloro-3-methyl-4-(3H-1,2,3-triazol-4-yl)-1-{[2-(trimethylsilyl)ethoxy]methyl}indazole (880.0 mg, 2.418 mmol, 1.0 equiv) in DMF (18 mL) was added NaH (145.1 mg, 3.627 mmol, 1.5 equiv) at 0° C. The mixture was stirred for 15 min at 0° C. CH₃I (514.8 mg, 3.627 mmol, 1.5 equiv) was added and the mixture was allowed to warm to room temperature and stirred for 2 hr. The resulting mixture was quenched with ice water and extracted with EA (2×50 mL). The combined organic layers were washed with water (100 mL) and brine (100 mL), dried over anhydrous Na₂SO₄. After filtration, the filtrate was concentrated under reduced pressure. The residue was purified by silica gel column chromatography, eluted with PE/EA (3:1) to afford 7-chloro-3-methyl-4-(2-methyl-1,2,3-triazol-4-yl)-1-{[2-(trimethylsilyl)ethoxy]methyl}indazole (330.0 mg, C76) as an oil that was taken to the next step without further purification. LCMS (ES, m/z): 378.2[M−H]⁻

### Synthesis of Intermediate C77

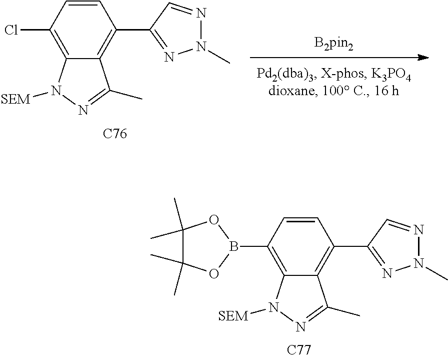

To a stirred mixture of 7-chloro-3-methyl-4-(2-methyl-1,2,3-triazol-4-yl)-1-{[2-(trimethylsilyl)ethoxy]methyl}indazole (280.0 mg, 0.741 mmol, 1.0 equiv) and 4,4,5,5-tetramethyl-2-(tetramethyl-1,3,2-dioxaborolan-2-yl)-1,3,2-dioxaborolane (282.2 mg, 1.111 mmol, 1.5 equiv) in dioxane (6 mL) were added K₃PO₄ (314.51 mg, 1.482 mmol, 2.0 equiv), X-phos (70.6 mg, 0.148 mmol, 0.2 equiv) and Pd₂(dba)₃ (67.8 mg, 0.074 mmol, 0.1 equiv) at room temperature under N₂ atmosphere. The resulting mixture was stirred for 16 hr at 100° C. under N₂ atmosphere. The resulting mixture was concentrated under vacuum. The residue was purified by silica gel column chromatography, eluted with PE/EA (3/1) to afford 3-methyl-4-(2-methyl-1,2,3-triazol-4-yl)-7-(4,4,5,5-tetramethyl-1,3,2-dioxaborolan-2-yl)-1-{[2-(trimethylsilyl)ethoxy]methyl}indazole (180.0 mg, C77) as an oil that was taken to the next step without further purification. LCMS (ES, m/z): 470.3[M+H]⁺

### Synthesis of Intermediate C78

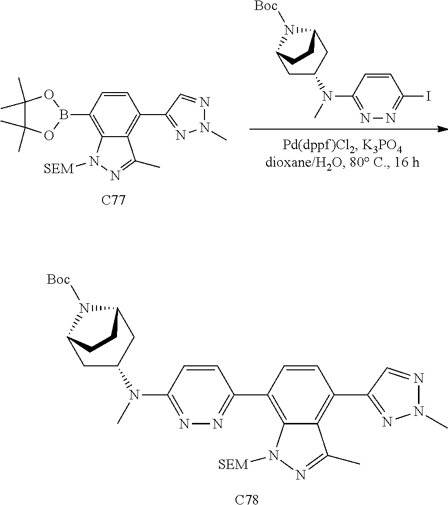

To a stirred solution of 3-methyl-4-(2-methyl-1,2,3-triazol-4-yl)-7-(4,4,5,5-tetramethyl-1,3,2-dioxaborolan-2-yl)-1-{[2-(trimethylsilyl)ethoxy]methyl}indazole (150.0 mg, 0.320 mmol, 1.0 equiv) and tert-butyl (1R,3S,5S)-3-[(6-iodopyridazin-3-yl)(methyl)amino]-8-azabicyclo[3.2.1]octane-8-carboxylate (141.9 mg, 0.320 mmol, 1.0 equiv) in dioxane/H₂O (2.3 mL, 5/1) was added K₃PO₄ (203.5 mg, 0.960 mmol, 3.0 equiv) (132.47 mg, 0.960 mmol, 3.0 equiv) and Pd(dppf)Cl₂·CH₂Cl₂ (26.0 mg, 0.032 mmol, 0.1 equiv) at room temperature under N₂ atmosphere. The resulting mixture was stirred for 2 hr at 80° C. under N₂ atmosphere. The resulting mixture was concentrated under reduced pressure. The residue was purified by silica gel column chromatography, eluted with PE/EA (1:1) to afford tert-butyl (1R,3S,5S)-3-[methyl({6-[3-methyl-4-(2-methyl-1,2,3-triazol-4-yl)-1-{[2-(trimethylsilyl)ethoxy]methyl}indazol-7-yl]pyridazin-3-yl})amino]-8-azabicyclo[3.2.1]octane-8-carboxylate (66.0 mg, C78) as an oil that was taken to the next step without further purification. LCMS (ES, m/z): 660.5[M+H]⁺

### Synthesis of Compound 375

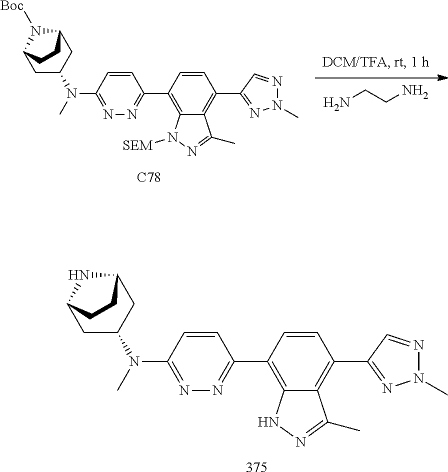

To a stirred solution of tert-butyl (1R,3S,5S)-3-[methyl({6-[3-methyl-4-(2-methyl-1,2,3-triazol-4-yl)-1-{[2-(trimethylsilyl)ethoxy]methyl}indazol-7-yl]pyridazin-3-yl})amino]-8-azabicyclo[3.2.1]octane-8-carboxylate (66.0 mg, 0.100 mmol, 1.0 equiv) in DCM (1.2 mL) was added TFA (0.4 mL) at room temperature under N₂ atmosphere. The resulting mixture was stirred for 1 hr at room temperature. The resulting mixture was concentrated under vacuum. The resulting was added ethylenediamine (132 mg) and stirred for 1 hr at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 20% to 55% gradient in 7 min; detector, UV 254 nm) to afford (1R,3S,5S)-N-methyl-N-{6-[3-methyl-4-(2-methyl-1,2,3-triazol-4-yl)-1H-indazol-7-yl]pyridazin-3-yl}-8-azabicyclo[3.2.1]octan-3-amine (21.3 mg, Compound 375) as a solid. LCMS (ES, m/z): 430.2[M+H] ¹H NMR (400 MHz, DMSO-d₆) δ 12.80 (s, 1H), 8.17 (d, J=9.8 Hz, 1H), 8.12 (s, 1H), 7.93 (d, J=7.6 Hz, 1H), 7.29 (d, J=7.5 Hz, 1H), 7.25 (d, J=9.8 Hz, 1H), 5.06 (t, J=5.9 Hz, 1H), 4.28 (s, 3H), 3.51-3.50 (m, 2H), 2.98 (s, 3H), 2.42 (s, 3H), 1.88-1.73 (m, 6H), 1.57 (dt, J=11.9, 4.3 Hz, 2H).

### Example 104: Synthesis of Compound 376

### Synthesis of Intermediate C79

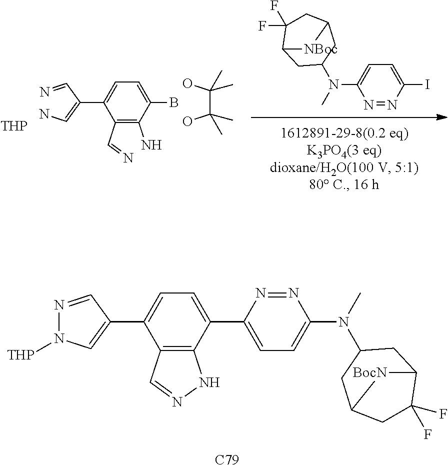

To a stirred solution of 4-[1-(oxan-2-yl)pyrazol-4-yl]-7-(4,4,5,5-tetramethyl-1,3,2-dioxaborolan-2-yl)-1H-indazole (50 mg, 0.127 mmol, 1 eq.) and tert-butyl 6,6-difluoro-3-[(6-iodopyridazin-3-yl) (methyl) amino]-8-azabicyclo[3.2.1]octane-8-carboxylate (91.36 mg, 0.191 mmol, 1.5 equiv) in dioxane (5 mL) and water (1 mL) were added Pd-PEPPSI-IPentC1 2-methylpyridine (o-picoline (10.67 mg, 0.013 mmol, 0.1 eq.) and tripotassium phosphate (80.75 mg, 0.381 mmol, 3 eq.) dropwise for 16 hr at 80° C. under a nitrogen atmosphere. The mixture was allowed to cool to room temperature. The resulting mixture was extracted with ethyl acetate (2×20 mL). The combined organic layers were washed with water (3×20 mL), dried over anhydrous sodium sulfate. After filtration, the filtrate was concentrated under reduced pressure. The residue was purified by silica gel column chromatography, eluted with PE/EA (1:1) to afford tert-butyl 6,6-difluoro-3-[methyl (6-{4-[1-(oxan-2-yl)pyrazol-4-yl]-1H-indazol-7-yl}pyridazin-3-yl)amino]-8-azabicyclo[3.2.1]octane-8-carboxylate (70 mg, C79) as a solid that was taken to the next step without further purification. LCMS (ES, m/z): 621 [M+H]

### Synthesis of Compound 376

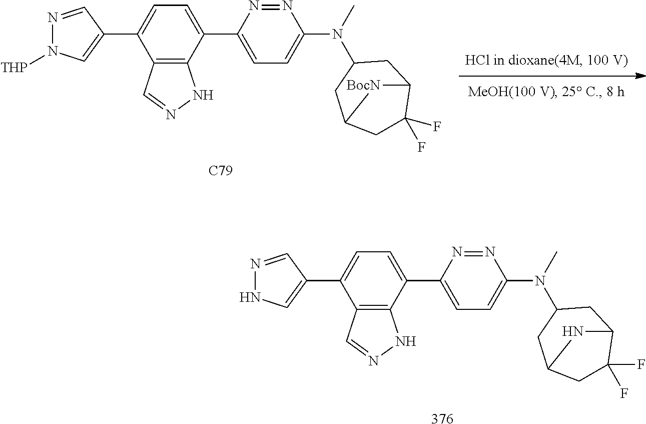

A solution of tert-butyl 6,6-difluoro-3-[methyl (6-{4-[1-(oxan-2-yl)pyrazol-4-yl]-1H-indazol-7-yl} pyridazin-3-yl)amino]-8-azabicyclo[3.2.1]octane-8-carboxylate (60 mg) and hydrochloric acid (gas)in 1,4-dioxane (6 mL) in methanol (6 mL) was stirred for 8 hr at 25° C. The resulting mixture was concentrated under reduced pressure. The crude product was purified by Prep-HPLC (Condition 7, Gradient 5) to afford 6,6-difluoro-N-methyl-N-{6-[4-(1H-pyrazol-4-yl)-1H-indazol-7-yl]pyridazin-3-yl}-8-azabicyclo[3.2.1]octan-3-amine (28.3 mg, Compound 376) as a solid. LCMS (ES, m/z): 437 [M+H] ¹H NMR (400 MHz, DMSO-d₆) δ 13.18 (s, 1H), 13.05 (s, 1H), 8.51 (d, J=1.6 Hz, 1H), 8.42 (s, 1H), 8.19 (d, J=9.8 Hz, 1H), 8.18 (s, 1H), 7.89 (d, J=7.8 Hz, 1H), 7.47 (d, J=7.6 Hz, 1H), 7.22 (d, J=9.8 Hz, 1H), 5.19 (s, 1H), 3.66 (s, 1H), 3.47 (d, J=13.4 Hz, 1H), 3.03 (s, 3H), 2.91 (s, 1H), 2.36 (s, 1H), 2.34 (s, 1H) 2.01-1.86 (m, 1H), 1.82 (s, 2H), 1.73 (dd, J=12.1, 5.2 Hz, 1H). ¹⁹F NMR (376 MHz, DMSO-d₆) δ-85.12, -85.72, -111.20, -111.80.

### Example 105: Synthesis of Compound 378

### Synthesis of Intermediate C80

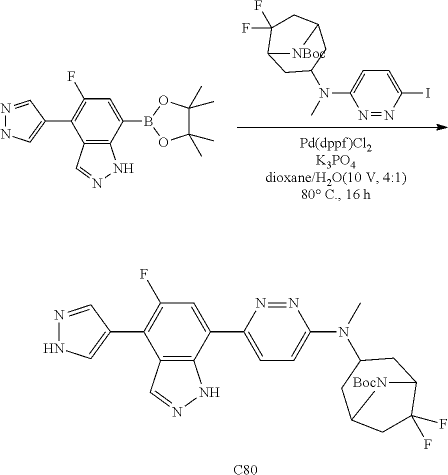 [SMILES: CC1(C)OB(c2cc(F)c(-c3cn[nH]c3)c3cn[nH]c23)OC1(C)C ; CN(c1ccc(I)nn1)C1CC2CC(F)(F)C(C1)N2C(=O)OC(C)(C)C ; CN(c1ccc(-c2cc(F)c(-c3cn[nH]c3)c3cn[nH]c23)nn1)C1CC2CC(F)(F)C(C1)N2C(=O)OC(C)(C)C ; "C80"]

To a stirred solution of 5-fluoro-4-(1H-pyrazol-4-yl)-7-(4,4,5,5-tetramethyl-1,3,2-dioxaborolan-2-yl)-1H-indazole (50 mg, 0.152 mmol, 1.00 eq.) and tert-butyl 6,6-difluoro-3-[(6-iodopyridazin-3-yl)(methyl) amino]-8-azabicyclo[3.2.1]octane-8-carboxylate (87.82 mg, 0.182 mmol, 1.2 eq.) in dioxane (10 mL) and water (2.5 mL) were added Pd(dppf)Cl₂ CH₂C12 (24.82 mg, 0.030 mmol, 0.2 equiv) and tripotassium phosphate (97.03 mg, 0.456 mmol, 3 eq.) dropwise for 16 hr at 80° C. under a nitrogen atmosphere. The mixture was allowed to cool to 25° C. The resulting mixture was extracted with ethyl acetate (3×10 mL). The combined organic layers were washed with water (3×15 mL), dried over anhydrous sodium sulfate. After filtration, the filtrate was concentrated under reduced pressure. The residue was purified by silica gel column chromatography, eluted with petroleum ether/ethyl acetate (3:1) to afford tert-butyl6,6-difluoro-3-({6-[5-fluoro-4-(1H-pyrazol-4-yl)-1H-indazol-7-yl]pyridazin-3-yl}(methyl)amino)-8-azabicyclo[3.2.1]octane-8-carboxylate (40 mg, C80) as a solid that was taken to the next step without further purification. LCMS (ES, m/z): 555 [M+H]

### Synthesis of Compound 378

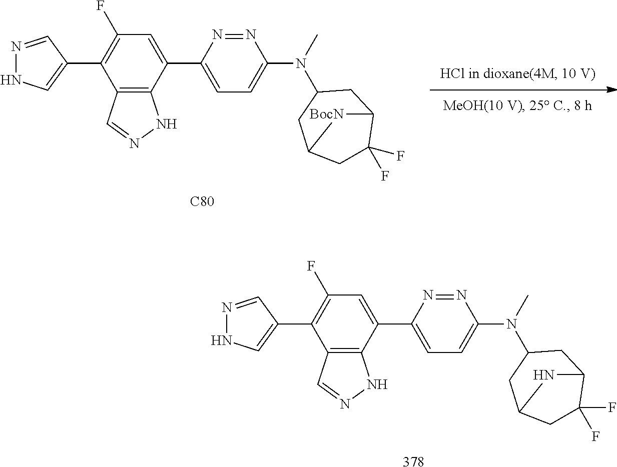 [SMILES: CN(c1ccc(-c2cc(F)c(-c3cn[nH]c3)c3cn[nH]c23)nn1)C1CC2CC(F)(F)C(C1)N2C(=O)OC(C)(C)C ; "C80" ; CN(c1ccc(-c2cc(F)c(-c3cn[nH]c3)c3cn[nH]c23)nn1)C1CC2CC(F)(F)C(C1)N2 ; "378"]

A solution of tert-butyl 6,6-difluoro-3-({6-[5-fluoro-4-(1H-pyrazol-4-yl)-1H-indazol-7-yl]pyridazin-3-yl}(methyl) amino)-8-azabicyclo[3.2.1]octane-8-carboxylate (60 mg, 0.108 mmol, 1.00 equiv) and hydrochloric acid (gas)in 1,4-dioxane (5 mL) in methanol (5 mL) was stirred for 8 hr at 25° C. The resulting mixture was concentrated under reduced pressure. The crude product was purified by preparative HPLC (Condition 4, Gradient 11) to afford6,6-difluoro-N-{6-[5-fluoro-4-(1H-pyrazol-4-yl)-1H-indazol-7-yl]pyridazin-3-yl}-N-methyl-8-azabicyclo[3.2.1]octan-3-amine (6.1 mg, Compound 378) as a solid. LCMS (ES, m/z): 455 [M+H] 1H NMR (400 MHz, DMSO-d₆) δ 13.32 (s, 1H), 13.17 (s, 1H), 8.49 (d, J=1.6 Hz, 1H), 8.43 (s, 1H), 8.23 (d, J=9.8 Hz, 1H), 8.15 (s, 1H), 7.93 (d, J=12.6 Hz, 1H), 7.22 (d, J=9.8 Hz, 1H), 5.22 (s, 1H), 3.66 (s, 1H), 3.48-3.45 (m, 1H), 3.04 (s, 3H), 2.91 (s, 1H), 2.36 (s, 2H), 1.96 (t, J=12.5 Hz, 1H), 1.91-1.80 (m, 2H), 1.73 (d, J=12.5 Hz, 1H) ¹⁹F NMR (376 MHz, DMSO-d₆) δ-85.44 (d, J=226.1 Hz), -111.48 (d, J=226.2 Hz), -125.13, -220.59.

### Example 106: Synthesis of Compound 379

### Synthesis of Intermediate C81

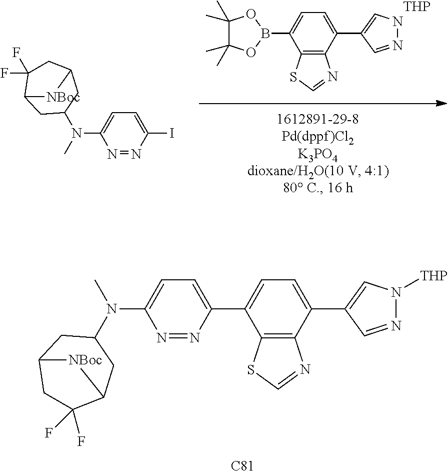

To a stirred solution of tert-butyl 6,6-difluoro-3-[(6-iodopyridazin-3-yl)(methyl)amino]-8-azabicyclo[3.2.1]octane-8-carboxylate (50 mg, 0.104 mmol, 1.00 eq.) and 4-[1-(oxan-2-yl)pyrazol-4-yl]-7-(4,4,5,5-tetramethyl-1,3,2-dioxaborolan-2-yl)-1,3-benzothiazole (64.23 mg, 0.156 mmol, 1.5 equiv) in 1,4-dioxane (5 mL) and water (1.25 mL) were added Pd-PEPPSI-IPentCl 2-methylpyridine (o-picoline (17.51 mg, 0.021 mmol, 0.2 equiv) and tripotassium phosphate (66.29 mg, 0.312 mmol, 3 equiv) dropwise for 16 hr at 80° C. under a nitrogen atmosphere. The mixture was allowed to cool to 25° C. The resulting mixture was extracted with ethyl acetate (3×10 mL). The combined organic layers were washed with water (3×15 mL), dried over anhydrous sodium sulfate. After filtration, the filtrate was concentrated under reduced pressure. The residue was purified by silica gel column chromatography, eluted with dichloromethane/methanol(15:1) to afford tert-butyl 6,6-difluoro-3-[methyl(6-{4-[(1E)-1-(oxan-2-ylamino)prop-1-en-2-yl]-1,3-benzothiazol-7-yl}pyridazin-3-yl)amino]-8-azabicyclo[3.2.1]octane-8-carboxylate (70 mg, C81) as a solid that was taken to the next step without further purification. LCMS (ES, m/z): 638 [M+H]

### Synthesis of Compound 379

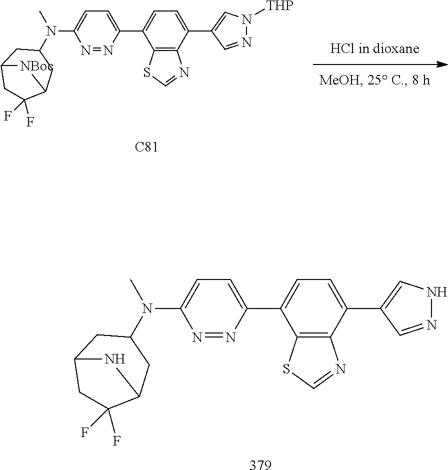

A solution of tert-butyl 6,6-difluoro-3-[methyl(6-{4-[1-(oxan-2-yl)pyrazol-4-yl]-1,3-benzothiazol-7-yl}pyridazin -3-yl)amino]-8-azabicyclo[3.2.1]octane-8-carboxylate (50 mg, 1 eq.) and hydrochloric acid(gas)in 1,4-dioxane (5 mL) in methanol (5 mL) was stirred for 8 hr at 25° C. The resulting mixture was concentrated under reduced pressure. The crude product was purified by preparative HPLC (Condition 7, Gradient 2) to afford 6,6-difluoro-N-methyl-N-{6-[4-(1H-pyrazol-4-yl)-1,3-benzothiazol-7-yl] pyridazin-3-yl}-8-azabicyclo [3.2.1]octan-3-amine (13 mg, Compound 379) as a solid. LCMS (ES, m/z): 454 [M+H] ¹H NMR 400 MHz, DMSO-d₆) δ 13.09 (s, 1H), 9.51 (s, 1H), 8.68 (s, 1H), 8.41 (s, 1H), 8.29 (d, J=9.9 Hz, 1H), 8.11 (d, J=8.1 Hz, 1H), 7.99 (d, J=8.0 Hz, 1H), 7.22 (d, J=9.8 Hz, 1H), 5.19 (s, 1H), 3.66 (s, 1H), 3.47 (d, J=13.0 Hz, 1H), 3.02 (s, 3H), 2.92 (s, 1H), 2.37 (d, J=13.9 Hz, 2H), 1.96 (t, J=11.4 Hz, 1H), 1.85-1.82 (m, 2H), 1.77-1.69 (m, 1H). ¹⁹F NMR (376 MHz, DMSO-d₆) δ-85.40 (d, J=226.0 Hz), -111.43 (d, J=226.4 Hz), -212.86.

### Example 107: Synthesis of Compound 380

### Synthesis of Intermediate C82

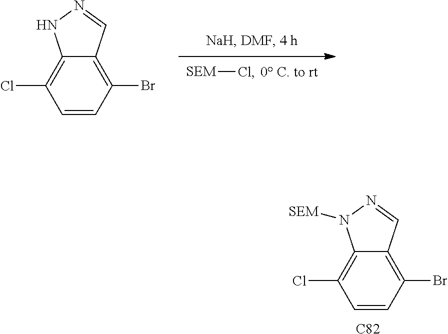 [SMILES: Clc1ccc(Br)c2cn[nH]c12 ; C[Si](C)(C)CCOCCl ; C[Si](C)(C)CCOCn1ncc2c(Br)ccc(Cl)c21 ; "C82"]

To a stirred solution of 4-bromo-7-chloro-1H-indazole (2 g, 8.640 mmol, 1.00 equiv) in DM F(20 mL) was added NaH (0.3 g, 12.960 mmol, 1.5 equiv) in portions at 0° C. under nitrogen at mosphere. The resulting mixture was stirred for 30 min at 0° C. under nitrogen atmosphere. To th e above mixture was added SEM-C1 (2.16 g, 12.960 mmol, 1.5 equiv) dropwise at 0° C. The resulting mixture was stirred for 4 h at room temperature and quenched by the addition of water (4 0 mL) at 0° C. The resulting mixture was extracted with EtOAc (3×20 mL). The combined organic layers were washed with water (100 mL), dried over anhydrous Na₂SO₄. After filtration, the filtrate was concentrated under reduced pressure. The residue was purified by silica gel column chromatography, eluted with PE/EA (10:1) to afford 4-bromo-7-chloro-1-{[2-(trimethylsilyl)ethox y]methyl}indazole (1.2 g, 38.39%) as oil that was taken to the next step without further purification. LCMS (ES, m/z): 361 [M+H]⁺

### Synthesis of Intermediate C83

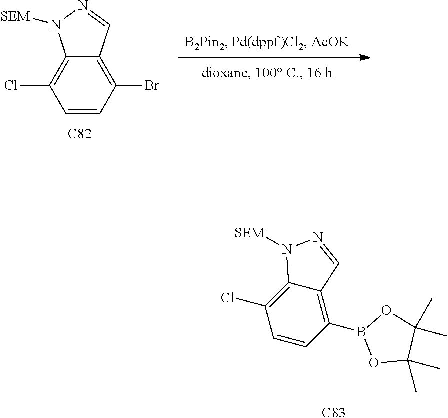 [SMILES: C[Si](C)(C)CCOCn1ncc2c(Br)ccc(Cl)c21 ; "C82" ; CC1(C)OB(c2ccc(Cl)c3c2cnn3COCC[Si](C)(C)C)OC1(C)C ; "C83"]

Into a 40 mL vial were added 4-bromo-7-chloro-1-{[2-(trimethylsilyl)ethoxy]methyl}indazole (600 mg, 1.659 mmol, 1.0 equiv), dioxane (10 ml), 4,4,5,5-tetramethyl-2-(tetramethyl-1,3,2-dioxaborolan-2-yl)-1,3,2-dioxaborolane (505.44 mg, 1.991 mmol, 1.2 equiv), potassium acetate (4 88.35 mg, 4.977 mmol, 3.0 equiv) and Pd(dppf)Cl₂ (135.12 mg, 0.166 mmol, 0.1 equiv) at room temperature under nitrogen atmosphere. The resulting mixture was stirred for 16 hr at 100° C. under nitrogen atmosphere. The mixture was concentrated under reduced pressure. The residue was purified by silica gel column chromatography, eluted with PE/EA (3:1) to afford 7-chloro-4-(4, 4,5,5-tetramethyl-1,3,2-dioxaborolan-2-yl)-1-{[2-(trimethylsilyl)ethoxy]methyl}indazole (400 m g, C83) as oil that was taken to the next step without further purification. LCMS (ES, m/z): 409 [M+H]⁺

### Synthesis of Intermediate C84

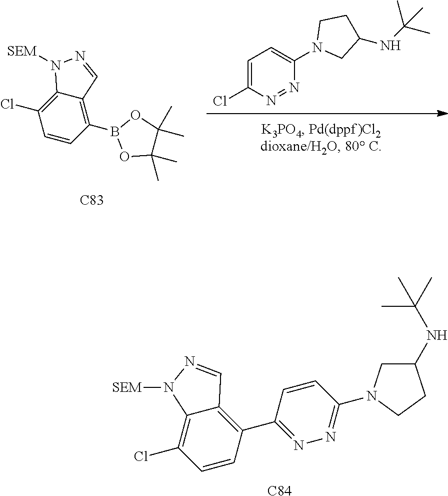 [SMILES: CC1(C)OB(c2ccc(Cl)c3c2cnn3COCC[Si](C)(C)C)OC1(C)C ; CC(C)(C)NC1CCN(c2ccc(Cl)nn2)C1 ; "C83" ; CC(C)(C)NC1CCN(c2ccc(-c3ccc(Cl)c4c3cnn4COCC[Si](C)(C)C)nn2)C1 ; "C84"]

Into a 40 mL vial were added 7-chloro-4-(4,4,5,5-tetramethyl-1,3,2-dioxaborolan-2-yl)-1-{[2-(trimethylsilyl)ethoxy]methyl}indazole (150 mg, 0.367 mmol, 1.00 equiv), dioxane (2 mL), water (0.4 mL),N-tert-butyl-1-(6-chloropyridazin-3-yl)pyrrolidin-3-amine (93.48 mg, 0.367 mmol, 1.0 equiv), K₃PO₄ (155.77 mg, 0.734 mmol, 2.0 equiv) and Pd(dppf)Cl₂ (29.89 mg, 0.037 mmol, 0.1 equiv) at room temperature under nitrogen atmosphere. The resulting mixture was stirred for 16 h at 80° C. under nitrogen atmosphere. The mixture was concentrated under reduced pressure. The residue was purified by silica gel column chromatography, eluted with CH₂Cl₂/MeOH (8:1) to afford N-tert-butyl-1-[6-(7-chloro-1-{[2-(trimethylsilyl)ethoxy]methyl}indazol-4-yl)pyridazin-3-yl]pyrrolidin-3-amine (120 mg, C84) as a solid that was taken to the next step without further purification. LCMS (ES, m/z): 501 [M+H]⁺

### Synthesis of Intermediate C85

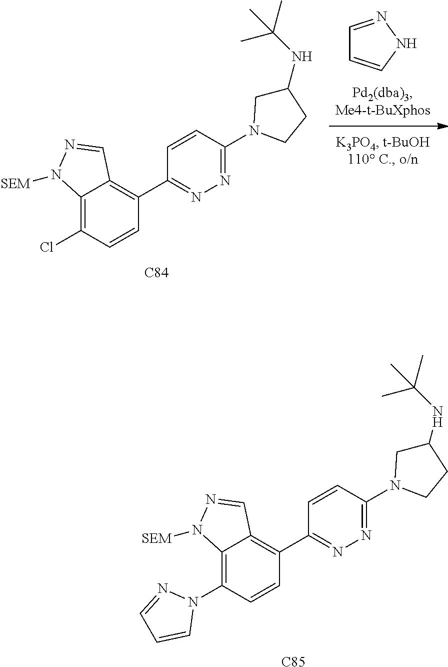 [SMILES: CC(C)(C)NC1CCN(c2ccc(-c3ccc(Cl)c4c3cnn4COCC[Si](C)(C)C)nn2)C1 ; c1cn[nH]c1 ; "C84" ; CC(C)(C)NC1CCN(c2ccc(-c3ccc(-n4cccn4)c4c3cnn4COCC[Si](C)(C)C)nn2)C1 ; "C85"]

Into an 8 mL vial were added N-tert-butyl-1-[6-(7-chloro-1-{[2-(trimethylsilyl)ethoxy]meth yl}indazol-4-yl)pyridazin-3-yl]pyrrolidin-3-amine (100 mg, 0.200 mmol, 1.00 equiv), t-BuOH (1 mL), pyrazole (16.30 mg, 0.240 mmol, 1.2 equiv), K₃PO₄ (84.71 mg, 0.400 mmol, 2.0 equiv), di-tert-butyl([2,3,4,5-tetramethyl-6-[2,4,6-tris(propan-2-yl)phenyl]phenyl])phosphane (19.19 mg, 0.040 mmol, 0.2 equiv) and Pd₂(dba)₃ (18.27 mg, 0.020 mmol, 0.1 equiv) at room temperature un der nitrogen atmosphere. The resulting mixture was stirred overnight at 110° C. under nitrogen at mosphere. The mixture was concentrated under reduced pressure. The residue was purified by silica gel column chromatography, eluted with CH₂Cl₂/MeOH (7:1) to afford N-tert-butyl-1-{6-[7-(pyrazol-1-yl)-1-{[2-(trimethylsilyl)ethoxy]methyl}indazol-4-yl]pyridazin-3-yl}pyrrolidin-3-amine (90 mg, C85) as solid that was taken to the next step without further purification. LCMS (ES, m/z): 533 [M+H]⁺

### Synthesis of Compound 380

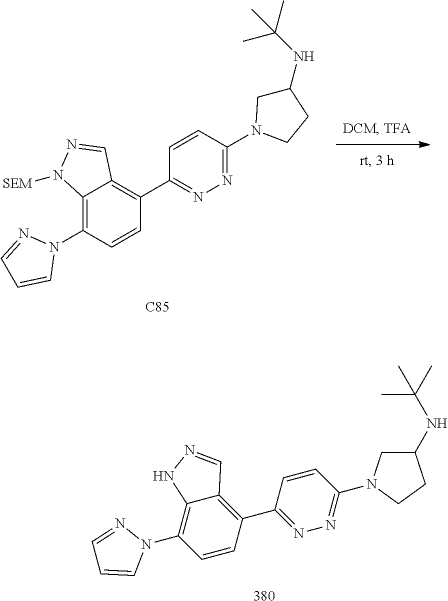 [SMILES: CC(C)(C)NC1CCN(c2ccc(-c3ccc(-n4cccn4)c4c3cnn4COCC[Si](C)(C)C)nn2)C1 ; "C85" ; CC(C)(C)NC1CCN(c2ccc(-c3ccc(-n4cccn4)c4[nH]ncc34)nn2)C1 ; "380"]

Into an 8 mL vial were added N-tert-butyl-1-{6-[7-(pyrazol-1-yl)-1-{[2-(trimethylsilyl)ethoxy]methyl}indazol-4-yl]pyridazin-3-yl}pyrrolidin-3-amine (80 mg, 0.150 mmol, 1.0 equiv), DC M (1 mL) and TFA (1 mL, 13.463 mmol, 89.66 equiv) at room temperature. The resulting mixture was stirred for 3 hr at room temperature. The resulting mixture was concentrated under reduced pressure. The residue product was purified by reverse phase flash with the following conditions (Column: Weich Ultimate XB-C18 50×250 mm 10 μm; Mobile Phase A: water (0.1% NH₃·H₂O), Mobile Phase B: ACN; Flow rate: 90 mL/min; Gradient: 15% B to 51% B in 12 min, 47% B; Wave Length: 220 nm; RT1(min):10.05;) to afford N-tert-butyl-1-{6-[7-(pyrazol-1-yl)-1H-indazol-4-yl]pyridazin-3-yl}pyrrolidin-3-amine (18 mg, Compound 380) as a solid. LCMS (ES, m/z): 403 [M+H]+ 1H NMR (300 MHz, DMSO-d6) δ 8.75 (s, 2H), 8.07 (d, J=9.6 Hz, 1H), 7.92 (d, J=1.8 Hz, 1H), 7.86 (d, J=7.9 Hz, 1H), 7.69 (d, J=8.0 Hz, 1H), 7.00 (d, J=9.6 Hz, 1H), 6.68 (t, J=2.2 Hz, 1H), 3.84 (d, J=9.4 Hz, 1H), 3.69 (s, 1H), 3.58 (s, 1H), 3.48 (d, J=9.0 Hz, 1H), 3.14 (s, 1H), 2.21 (s, 1H), 1.80 (s, 1H), 1.12 (s, 9H).

### Example 108: Synthesis of Compound 381

### Synthesis of Intermediate C86

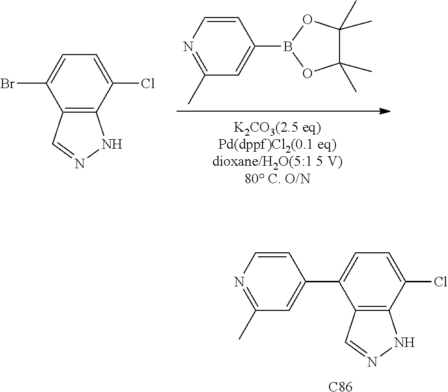 [SMILES: Clc1ccc(Br)c2cn[nH]c12 ; Cc1cc(B2OC(C)(C)C(C)(C)O2)ccn1 ; Cc1cc(-c2ccc(Cl)c3[nH]ncc23)ccn1 ; "C86"]

A mixture of 4-bromo-7-chloro-1H-indazole (200.00 mg, 0.8 mmol, 1.00 eq.), 2-methyl-4-(4,4,5,5-tetramethyl-1,3,2-dioxaborolan-2-yl) pyridine (189.29 mg, 0.8 mmol, 1.00 eq.), Pd(dppf)Cl₂ (63.22 mg, 0.08 mmol, 0.10 eq.) and potassium carbonate (298.53 mg, 2.1 mmol, 2.50 eq.) in dioxane (5.0 mL) and water (1.0 mL) was stirred overnight at 80° C. under a nitrogen atmosphere. The mixture was allowed to cool to 25° C. The resulting mixture was diluted with water (50.0 mL). The resulting mixture was extracted with ethyl acetate (2×50.0 mL), dried over anhydrous sodium sulfate. After filtration, the filtrate was concentrated under reduced pressure. The residue was purified by silica gel column chromatography, eluted with PE/EA (1:2) to afford 7-chloro-4-(2-methylpyridin-4-yl)-1H-indazole (220.00 mg, C86) as a solid that was taken to the next step without further purification. LCMS (ES, m/z): 244 [M+H]⁺

### Synthesis of Intermediate C87

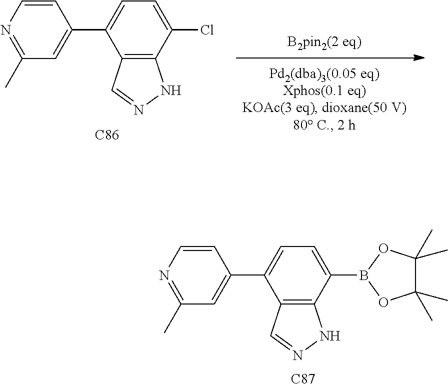 [SMILES: Cc1cc(-c2ccc(Cl)c3[nH]ncc23)ccn1 ; "C86" ; Cc1cc(-c2ccc(B3OC(C)(C)C(C)(C)O3)c3[nH]ncc23)ccn1 ; "C87"]

A mixture of 7-chloro-4-(2-methylpyridin-4-yl)-1H-indazole (230.00 mg, 0.9 mmol, 0.62 eq.), bis(pinacolato)diboron (383.48 mg, 1.5 mmol, 2.00 eq.), Pd₂(dba)₃ (34.57 mg, 0.03 mmol, 0.05 eq.), XPhos (35.99 mg, 0.07 mmol, 0.10 eq.) and potassium acetate (222.31 mg, 2.2 mmol, 3.00 eq.) in dioxane (11.5 mL) was stirred for 2 hr at 80° C. under a nitrogen atmosphere. The mixture was allowed to cool to 25° C. The resulting mixture was diluted with water (50.0 mL). The resulting mixture was extracted with ethyl acetate (2×50.0 mL), dried over anhydrous sodium sulfate. After filtration, the filtrate was concentrated under reduced pressure to afford crude 4-(2-methylpyridin-4-yl)-7-(4,4,5,5-tetramethyl-1,3,2-dioxaborolan-2-yl)-1H-indazole (602.00 mg, C87) as a semi-solid that was taken to the next step without further purification. LCMS (ES, m/z): 336 [M+H]⁺

### Synthesis of Intermediate C88

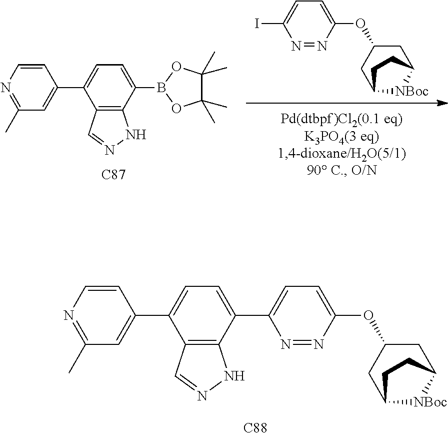

A mixture of 4-(2-methylpyridin-4-yl)-7-(4,4,5,5-tetramethyl-1,3,2-dioxaborolan-2-yl)-1H-indazole (65 mg, 0.194 mmol, 1.39 eq.), tert-butyl (exo)-3-[(6-iodopyridazin-3-yl) oxy]-8-azabicyclo [3.2.1] octane-8-carboxylate (60 mg, 0.139 mmol, 1.00 eq.), Pd(DtBPF)Cl₂ (9.07 mg, 0.014 mmol, 0.1 eq.) and tripotassium phosphate (88.59 mg, 0.417 mmol, 3 eq.) in dioxane (1.0 mL) and water (0.2 mL) was stirred for 16 hr at 90° C. under a nitrogen atmosphere. The mixture was allowed to cool down to 25° C. The resulting mixture was diluted with water (50.0 mL). The resulting mixture was extracted with ethyl acetate (2×50.0 mL), dried over anhydrous sodium sulfate. After filtration, the filtrate was concentrated under reduced pressure. The residue was purified by silica gel column chromatography, eluted with petroleum ether/ethyl acetate (1:5) to afford tert-butyl (exo)-3-({6-[4-(2-methylpyridin-4-yl)-1H-indazol-7-yl] pyridazin-3-yl} oxy)-8-azabicyclo [3.2.1] octane-8-carboxylate (63.00 mg, C88) as a solid that was taken to the next step without further purification. LCMS (ES, m/z): 513 [M+H]⁺

### Synthesis of Compound 381

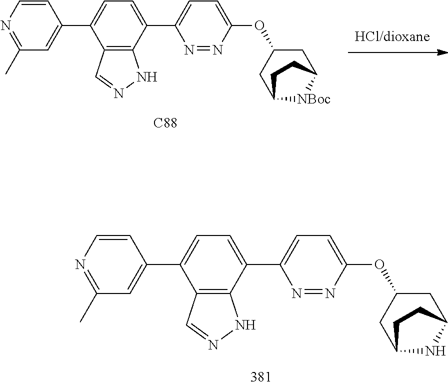

A solution of tert-butyl (exo)-3-({6-[4-(2-methylpyridin-4-yl)-1H-indazol-7-yl] pyridazin-3-yl} oxy)-8-azabicyclo [3.2.1] octane-8-carboxylate (40.00 mg, 0.07 mmol, 1.00 eq.) in hydrochloric acid(gas)in 1,4-dioxane (0.4 mL) and methanol (1.2 mL) was stirred for 1 hr at room temperature. The resulting mixture was concentrated under vacuum. The crude product was purified by preparative chiral HPLC (Condition 3, Gradient 2) to afford 7-{6-[(exo)-8-azabicyclo [3.2.1] octan-3-yloxy] pyridazin-3-yl}-4-(2-methylpyridin-4-yl)-1H-indazole (2.00 mg, C88) as a solid. LCMS (ES, m/z): 413 [M+H]⁺¹H NMR (400 MHz, DMSO-d₆) δ 13.57 (s, 1H), 9.09 (d, J=32.1 Hz, 2H), 8.88 (d, J=6.1 Hz, 1H), 8.55 (d, J=10.6 Hz, 2H), 8.32 (s, 1H), 8.24 (d, J=7.6 Hz, 2H), 7.75 (d, J=7.7 Hz, 1H), 7.48 (d, J=9.3 Hz, 1H), 5.69 (dq, J=11.0, 5.5 Hz, 1H), 4.17 (s, 2H), 3.76 (s, 2H), 2.86-2.81 (m, 3H), 2.05 (d, J=11.2 Hz, 6H).

### Example 109: Synthesis of Compound 382

### Synthesis of Intermediate C89

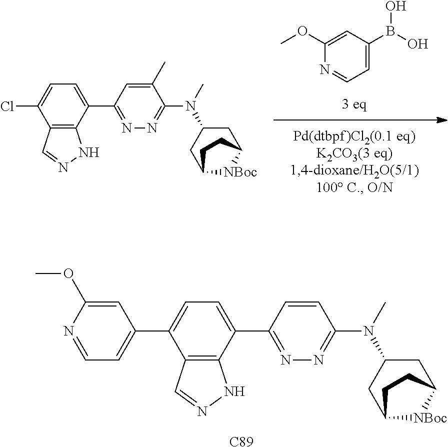

A solution of tert-butyl (exo)-3-{[6-(4-chloro-1H-indazol-7-yl)pyridazin-3-yl](methyl)amino}-8-azabicyclo[3.2.1]octane-8-carboxylate (40 mg, 0.085 mmol, 1.00 eq.), 2-methoxypyridin-4-ylboronic acid (39 mg, 0.255 mmol, 3 eq.), Pd(DtBPF)Cl₂ (5 mg, 0.008 mmol, 0.1 eq.) and potassium carbonate (35 mg, 0.255 mmol, 3 eq.) in 1,4-dioxane (0.5 mL) and water (0.1 mL) was stirred for 2 hr at 100° C. under a nitrogen atmosphere. The mixture was allowed to cool down to room temperature. The resulting mixture was extracted with ethyl acetate (3×5 mL), dried over anhydrous sodium sulfate. After filtration, the filtrate was concentrated under reduced pressure. The residue was purified by silica gel column chromatography, eluted with petroleum ether/ethyl acetate (1:1) to afford tert-butyl (exo)-3-({6-[4-(2-methoxypyridin-4-yl)-1H-indazol-7-yl]pyridazin-3-yl}(methyl)amino)-8-azabicyclo[3.2.1]octane-8-carboxylate as a solid that was taken to the next step without further purification. LCMS (ES, m/z):542 [M+H]⁺

### Synthesis of Compound 382

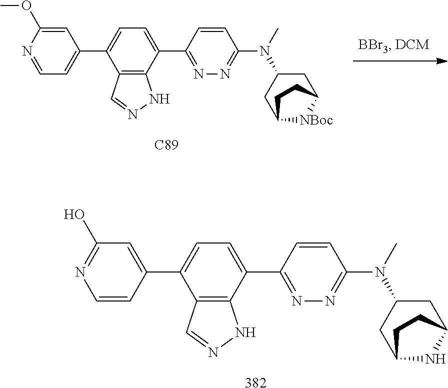

A solution of tert-butyl (exo)-3-({6-[4-(2-methoxypyridin-4-yl)-1H-indazol-7-yl]pyridazin-3-yl}(methyl)amino)-8-azabicyclo[3.2.1]octane-8-carboxylate (28 mg, 0.052 mmol, 1.00 eq.) and boron tribromide (0.02 mL, 0.260 mmol, 5 eq.) in dichloromethane (1 mL) was stirred for 16 hr at 80° C. The mixture was allowed to cool to room temperature. The reaction was quenched by the addition of methanol (5 mL) at room temperature. The mixture was adjusted to pH 6 with saturated sodium bicarbonate (aqueous). The resulting solution was decolorized by the addition of active carbon. The crude product was purified by preparative HPLC (Condition 4, Gradient 7) to afford 4-(7-{6-[(exo)-8-azabicyclo[3.2.1]octan-3-yl(methyl)amino]pyridazin-3-yl}-1H-indazol-4-yl)pyridin-2-ol (2.4 mg, Compound C82) as a solid. LCMS (ES, m/z): 428 [M+H]+¹H NMR (400 MHz, DMSO-d₆) δ 13.30 (s, 1H), 12.15-11.30 (s, 1H), 8.25 (s, 1H), 8.19 (d, J=9.9 Hz, 1H), 7.98 (d, J=7.8 Hz, 1H), 7.54 (d, J=6.8 Hz, 1H), 7.43 (d, J=7.6 Hz, 1H), 7.27 (d, J=9.8 Hz, 1H), 6.66 (d, J=1.8 Hz, 1H), 6.60 (dd, J=6.7, 1.9 Hz, 1H), 5.11-5.03 (m, 1H), 3.51 (s, 2H), 2.99 (s, 3H), 1.88-1.78 (m, 2H), 1.76 (s, 4H), 1.57 (d, J=11.1 Hz, 2H).

### Example 110: Synthesis of Compound 383

### Synthesis of Intermediate C90

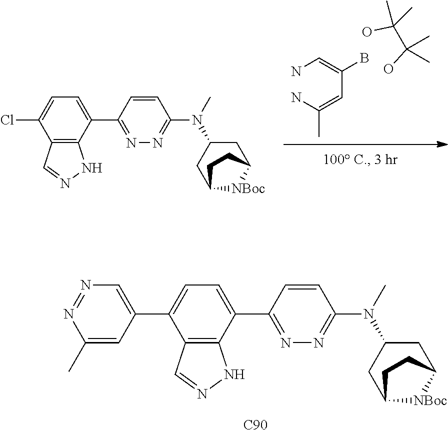

A solution of tert-butyl (xeo)-3-{[6-(4-chloro-1H-indazol-7-yl)pyridazin-3-yl](methyl)amino}-8-azabicyclo[3.2.1]octane-8-carboxylate (50 mg, 0.107 mmol, 1.00 equiv) and 3-methyl-5-(4,4,5,5-tetramethyl-1,3,2-dioxaborolan-2-yl)pyridazine (35 mg, 0.161 mmol, 1.5 equiv), Pd-PEPPSI-IPentCl 2-methylpyridine (o-picoline (9 mg, 0.011 mmol, 0.1 equiv) in 1,4-dioxane (0.5 mL) was stirred for 3 hr at 100° C. under nitrogen atmosphere. The mixture was allowed to cool down to room temperature. The resulting mixture was extracted with EA (3×5 mL). dried over anhydrous Na₂SO₄. After filtration, the filtrate was concentrated under reduced pressure. The residue was purified by silica gel column chromatography, eluted with PE/EA (1:1) to afford tert-butyl (exo)-3-[methyl({6-[4-(6-methylpyridazin-4-yl)-1H-indazol-7-yl]pyridazin-3-yl})amino]-8-azabicyclo[3.2.1]octane-8-carboxylate (55 mg, C90) as a solid that was taken to the next step without further purification. LCMS (ES, m/z): 527 [M+H]⁺

### Synthesis of Compound 383

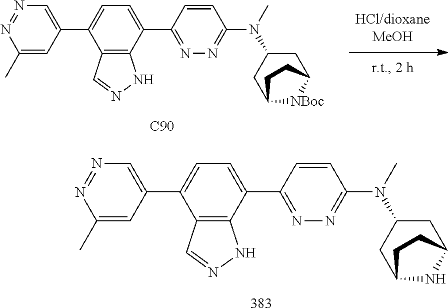

A solution of tert-butyl (exo)-3-[methyl({6-[4-(6-methylpyridazin-4-yl)-1H-indazol-7-yl]pyridazin-3-yl})amino]-8-azabicyclo[3.2.1]octane-8-carboxylate (55 mg, 0.104 mmol, 1.00 eq.) in methanol (1 mL), hydrochloric acid(gas)in 1,4-dioxane (1 mL) was stirred for 2 hr at room temperature. The resulting mixture was concentrated under reduced pressure. The crude product was purified by preparative HPLC (Condition 4, Gradient 10) to afford (exo)-N-methyl-N-{6-[4-(6-methylpyridazin-4-yl)-1H-indazol-7-yl]pyridazin-3-yl}-8-azabicyclo[3.2.1]octan-3-amine (12.8 mg, Compound 383) as a solid. LCMS (ES, m/z): 427 [M+H] ¹H NMR (400 MHz, DMSO-d₆) δ 13.38 (s, 1H), 9.53 (d, J=2.2 Hz, 1H), 8.48 (s, 1H), 8.23 (d, J=9.8 Hz, 1H), 8.06 (d, J=7.7 Hz, 1H), 8.00 (d, J=2.2 Hz, 1H), 7.62 (d, J=7.6 Hz, 1H), 7.28 (d, J=9.8 Hz, 1H), 5.09 (s, 1H), 3.51 (s, 2H), 2.99 (s, 3H), 2.77 (s, 3H), 1.86 (d, 1H), 1.83-1.80 (m, 2H), 1.86-1.79 (m, 4H), 1.62-1.52 (m, 2H).

### Example 111: Synthesis of Compound 384

### Synthesis of Intermediate C91

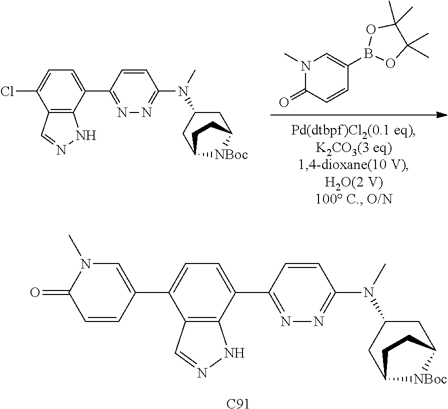

A solution of tert-butyl (exo)-3-{[6-(4-chloro-1H-indazol-7-yl)pyridazin-3-yl](methyl)amino}-8-azabicyclo[3.2.1]octane-8-carboxylate (60 mg, 0.128 mmol, 1.00 eq.) and 1-methyl-5-(4,4,5,5-tetramethyl-1,3,2-dioxaborolan-2-yl)pyridin-2-one (45 mg, 0.192 mmol, 1.5 equiv), Pd(DtBPF)Cl₂ (8 mg, 0.013 mmol, 0.1 eq.) and potassium carbonate (53 mg, 0.384 mmol, 3 eq.) in 1,4-dioxane (0.6 mL) and water (0.15 mL) was stirred for overnight at 100° C. under a nitrogen atmosphere. The mixture was allowed to cool to room temperature. The resulting mixture was extracted with ethyl acetate (3×5 mL), dried over anhydrous sodium sulfate. After filtration, the filtrate was concentrated under reduced pressure. The residue was purified by silica gel column chromatography, eluted with PE/EA (1:1) to afford tert-butyl (exo)-3-[methyl({6-[4-(1-methyl-6-oxopyridin-3-yl)-1H-indazol-7-yl]pyridazin-3-yl})amino]-8-azabicyclo[3.2.1]octane-8-carboxylate (63 mg, C91) as a solid that was taken to the next step without further purification. LCMS (ES, m/z): 542 [M+H]⁺

### Synthesis of Compound 384

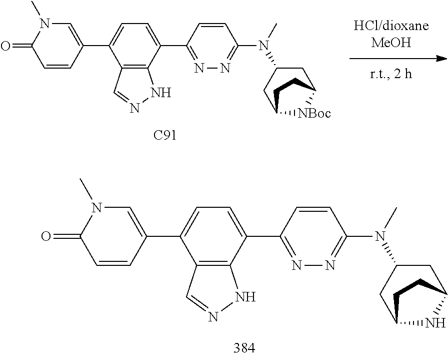

A solution of tert-butyl (exo)-3-[methyl({6-[4-(1-methyl-6-oxopyridin-3-yl)-1H-indazol-7-yl]pyridazin-3-yl})amino]-8-azabicyclo[3.2.1]octane-8-carboxylate (63 mg, 0.116 mmol, 1.00 eq.) in methanol (1 mL), hydrochloric acid(gas)in 1,4-dioxane (1 mL) was stirred for 2 hr at room temperature. The resulting mixture was concentrated under reduced pressure. The crude product was purified by preparative HPLC with the following conditions (Condition 7, Gradient 2) to afford 5-(7-{6-[(exo)-8-azabicyclo[3.2.1]octan-3-yl(methyl)amino]pyridazin-3-yl}-1H-indazol-4-yl)-1-methylpyridin-2-one (10.4 mg, Compound 384) as a solid that was taken to the next step without further purification. LCMS (ES, m/z): 442 [M+H]⁺¹H NMR (400 MHz, DMSO-d₆) δ 13.18 (s, 1H), 8.37 (s, 1H), 8.22 (d, J=2.7 Hz, 1H), 8.17 (d, J=9.8 Hz, 1H), 7.98-7.89 (m, 2H), 7.29 (d, J=15.4, 8.7 Hz, 1H), 7.26 (d, 1H) 6.58 (d, J=9.4 Hz, 1H), 5.10-5.03 (m, 1H), 3.59 (s, 3H), 3.51 (s, 2H), 2.98 (s, 3H), 2.17 (s, 1H), 1.82 (dd, 2H), 1.76 (s, 4H) 1.61-1.52 (m, 2H).

### Example 112: Synthesis of Compound 385

### Synthesis of Intermediate C92

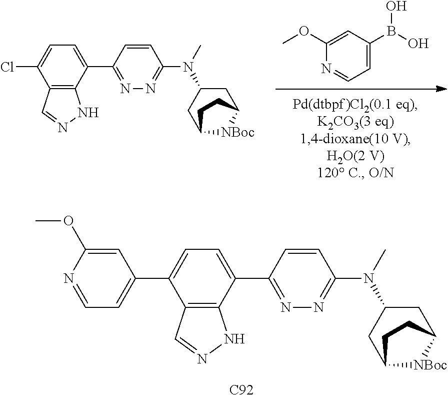

A solution of tert-butyl (exo)-3-{[6-(4-chloro-1H-indazol-7-yl)pyridazin-3-yl](methyl)amino}-8-azabicyclo[3.2.1]octane-8-carboxylate (50 mg, 0.107 mmol, 1.00 eq.), 2-methoxypyridin-4-ylboronic acid (49 mg, 0.321 mmol, 3 eq.), Pd(DtBPF)Cl₂ (7 mg, 0.011 mmol, 0.1 eq.) and potassium carbonate (44 mg, 0.321 mmol, 3 eq.) in 1,4-dioxane (0.5 mL) and water (0.1 mL) was stirred for overnight at 120° C. under a nitrogen atmosphere. The mixture was allowed to cool down to room temperature. The resulting mixture was extracted with ethyl acetate (3×5 mL), and dried over anhydrous sodium sulfate. After filtration, the filtrate was concentrated under reduced pressure. The residue was purified by silica gel column chromatography, eluted with petroleum ether/ethyl aceate/EA (1:1) to afford tert-butyl (exo)-3-({6-[4-(2-methoxypyridin-4-yl)-1H-indazol-7-yl]pyridazin-3-yl}(methyl)amino)-8-azabicyclo[3.2.1]octane-8-carboxylate (47 mg, C92) as a solid that was taken to the next step without further purification. LCMS (ES, m/z): 542 [M+H]⁺

### Synthesis of Compound 385

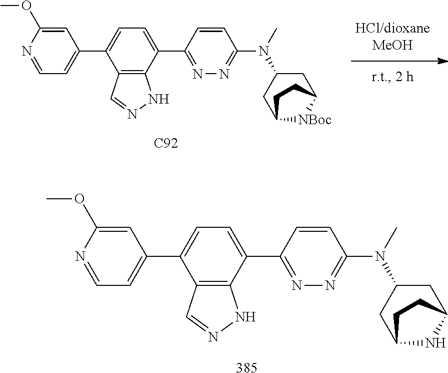

A solution of tert-butyl (exo)-3-({6-[4-(2-methoxypyridin-4-yl)-1H-indazol-7-yl]pyridazin-3-yl}(methyl)amino)-8-azabicyclo[3.2.1]octane-8-carboxylate (47 mg, 0.087 mmol, 1.00 eq.) in methanol (1 mL) and hydrochloric acid(gas)in 1,4-dioxane (1 mL) was stirred for 2 hr at room temperature. The resulting mixture was concentrated under reduced pressure. The crude product was purified by preparative HPLC (Condition 4, Gradient 11) to afford (exo)-N-{6-[4-(2-methoxypyridin-4-yl)-1H-indazol-7-yl]pyridazin-3-yl}-N-methyl-8-azabicyclo[3.2.1]octan-3-amine (10.1 mg, 26%) as a solid. LCMS (ES, m/z): 442 [M+H] ¹H NMR (400 MHz, DMSO-d₆) δ 13.30 (s, 1H), 8.37-8.29 (m, 2H), 8.20 (d, J=9.8 Hz, 1H), 8.01 (d, J=7.7 Hz, 1H), 7.48 (d, J=7.6 Hz, 1H), 7.42 (dd, J=5.3, 1.5 Hz, 1H), 7.27 (d, J=9.8 Hz, 1H), 7.18 (d, J=1.6 Hz, 1H), 5.08 (s, 1H), 3.95 (s, 3H), 3.51 (s, 2H), 2.99 (s, 3H), 1.83 (td, J=11.9, 2.9 Hz, 2H), 1.77 (d, J=2.4 Hz, 4H), 1.58 (dt, J=13.3, 4.6 Hz, 2H).

### Example 113: Synthesis of Compound 386

### Synthesis of Intermediate C93

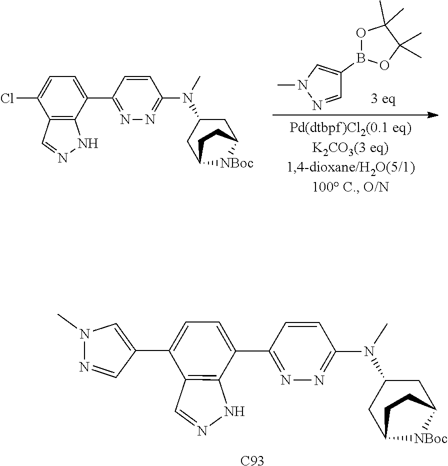

A solution of tert-butyl (exo)-3-{[6-(4-chloro-1H-indazol-7-yl)pyridazin-3-yl](methyl)amino}-8-azabicyclo[3.2.1]octane-8-carboxylate (50 mg, 0.107 mmol, 1.00 eq.),1-methyl-4-(4,4,5,5-tetramethyl-1,3,2-dioxaborolan-2-yl)pyrazole (66 mg, 0.321 mmol, 3 eq.), Pd(DtBPF)Cl₂ (7 mg, 0.011 mmol, 0.1 eq.) and potassium carbonate (44 mg, 0.321 mmol, 3 eq.) in 1,4-dioxane (0.5 mL) and water (0.1 mL) was stirred for overnight at 100° C. under nitrogen atmosphere. The mixture was allowed to cool down to room temperature. The resulting mixture was extracted with ethyl acetate (3×5 mL), dried over anhydrous sodium sulfate. After filtration, the filtrate was concentrated under reduced pressure. The residue was purified by silica gel column chromatography, eluted with petroleum ether/ethylacetate/EA (1:1) to afford tert-butyl (exo)-3-[methyl({6-[4-(1-methylpyrazol-4-yl)-1H-indazol-7-yl]pyridazin-3-yl})amino]-8-azabicyclo[3.2.1]octane-8-carboxylate (44 mg, C93) as a solid that was taken to the next step without further purification. LCMS (ES, m/z): 515 [M+H]⁺

### Synthesis of Compound 386

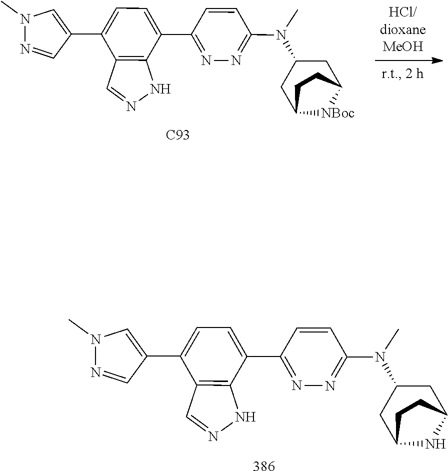

A solution of tert-butyl (exo)-3-[methyl({6-[4-(1-methylpyrazol-4-yl)-1H-indazol-7-yl]pyridazin-3-yl})amino]-8-azabicyclo[3.2.1]octane-8-carboxylate (44 mg, 0.085 mmol, 1.00 eq.) in methanol (1 mL), hydrochloric acid(gas)in 1,4-dioxane (1 mL) was stirred for 2 hr at room temperature. The resulting mixture was concentrated under reduced pressure. The crude product was purified by preparative HPLC (Condition 4, Gradient 10) to afford (exo)-N-methyl-N-{6-[4-(1-methylpyrazol-4-yl)-1H-indazol-7-yl]pyridazin-3-yl}-8-azabicyclo[3.2.1]octan-3-amine (3.5 mg, Compound 386) as a solid. LCMS (ES, m/z): 415 [M+H]⁺¹H NMR (400 MHz, DMSO-d₆) δ 13.09 (s, 1H), 8.48 (d, J=2.8 Hz, 2H), 8.18-8.09 (m, 2H), 7.89 (d, J=7.7 Hz, 1H), 7.43 (d, J=7.6 Hz, 1H), 7.24 (d, J=9.8 Hz, 1H), 5.03 (s, 1H), 3.96 (s, 3H), 3.51 (s, 2H), 2.97 (s, 3H), 2.20 (s, 1H), 1.84-1.76 (m, 2H), 1.76 (d, J=2.5 Hz, 4H), 1.56 (ddd, J=12.5, 5.9, 2.6 Hz, 2H).

### Example 114: Synthesis of Compound 389

### Synthesis of Intermediate C94

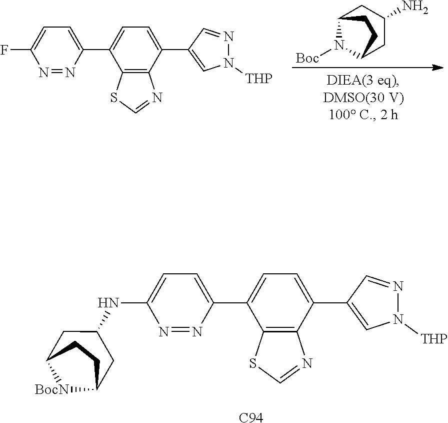

A mixture of 7-(6-fluoropyridazin-3-yl)-4-[1-(oxan-2-yl) pyrazol-4-yl]-1,3-benzothiazole (300 mg, 0.78 mmol, 1.00 eq.), tert-butyl (exo)-3-amino-8-azabicyclo [3.2.1] octane-8-carboxylate (196 mg, 0.86 mmol, 1.10 eq.) and DIEA (305 mg, 2.36 mmol, 3.00 eq.) in DMSO (9.0 mL) was stirred for 2 hr at 100° C. The mixture was allowed to cool down to room temperature. The resulting mixture was diluted with water (20 mL). The precipitated solids were collected by filtration and washed with water (2×20 mL). The resulting mixture was concentrated under vacuum and purified by preparative HPLC (Condition 4, Gradient 10) to afford tert-butyl (exo)-3-[(6-{4-[1-(oxan-2-yl) pyrazol-4-yl]-1,3-benzothiazol-7-yl} pyridazin-3-yl) amino]-8-azabicyclo [3.2.1] octane-8-carboxylate (412 mg, C94) as a solid. LCMS (ES, m/z): 588 [M+H]⁺

### Synthesis of Intermediate C95

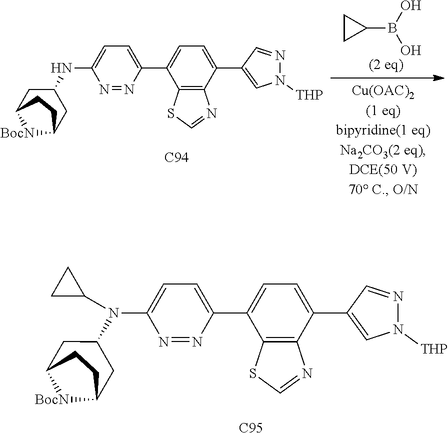

A mixture of tert-butyl (exo)-3-[(6-{4-[1-(oxan-2-yl) pyrazol-4-yl]-1,3-benzothiazol-7-yl}pyridazin-3-yl) amino]-8-azabicyclo [3.2.1] octane-8-carboxylate (410 mg, 0.69 mmol, 1.00 eq.), cyclopropylboronic acid (120 mg, 1.39 mmol, 2.00 eq.), bipyridyl (109 mg, 0.69 mmol, 1.00 eq.), copper acetate (127 mg, 0.69 mmol, 1.00 eq.) and sodium carbonate(148 mg, 1.39 mmol, 2.00 eq.) in dichloroethane (20 mL) was stirred overnight at 70° C. under an oxygen atmosphere. The mixture was allowed to cool to room temperature. The resulting mixture was diluted with water (50 mL). The resulting mixture was extracted with ethyl acetate (2×50 mL), dried over anhydrous sodium sulfate. After filtration, the filtrate was concentrated under reduced pressure. The residue was purified by silica gel column chromatography, eluted with petroleum ether/ethyl acetate (1:2) to afford tert-butyl (exo)-3-[cyclopropyl(6-{4-[1-(oxan-2-yl) pyrazol-4-yl]-1,3-benzothiazol-7-yl} pyridazin-3-yl) amino]-8-azabicyclo [3.2.1] octane-8-carboxylate (120 mg, C95) as an oil that was taken to the next step without further purification. LCMS (ES, m/z): 628 [M+H]⁺

### Synthesis of Compound 389

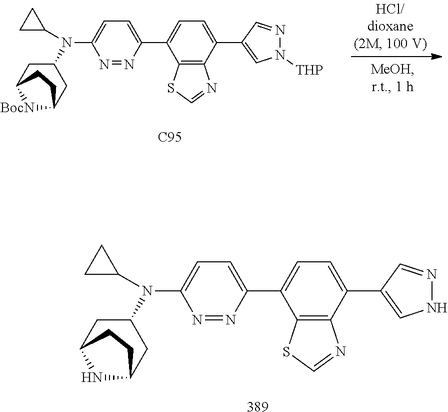

A solution of tert-butyl (exo)-3-[cyclopropyl(6-{4-[1-(oxan-2-yl) pyrazol-4-yl]-1,3-benzothiazol-7-yl} pyridazin-3-yl) amino]-8-azabicyclo [3.2.1] octane-8-carboxylate (120 mg, 0.19 mmol, 1.00 eq.) in hydrochloric acid(gas)in 1,4-dioxane (0.8 mL) and methanol (2.3 mL) was stirred for 1 hr at room temperature. The resulting mixture was concentrated under vacuum. The crude product was purified by preparative chiral HPLC (Condition 2, Gradient 2) to afford (exo)-N-cyclopropyl-N-{6-[4-(1H-pyrazol-4-yl)-1,3-benzothiazol-7-yl] pyridazin-3-yl}-8-azabicyclo [3.2.1] octan-3-amine) as a solid. LCMS (ES, m/z): 444 [M+H]⁺¹H NMR (400 MHz, DMSO-d₆) δ 13.01 (s, 1H), 9.49 (s, 1H), 8.52 (s, 2H), 8.25 (d, J=9.7 Hz, 1H), 8.09 (d, J=8.0 Hz, 1H), 7.98 (d, J=8.0 Hz, 1H), 7.48 (d, J=9.7 Hz, 1H), 4.90 (tt, J=11.7, 5.4 Hz, 1H), 3.52 (s, 2H), 2.11 (td, J=12.1, 3.0 Hz, 2H), 1.98 (s, 1H), 1.80-1.72 (m, 6H), 1.01 (td, J=6.9, 4.9 Hz, 2H), 0.72-0.64 (m, 2H).

### Example 115: Synthesis of Compound 390

### Synthesis of Intermediate C96

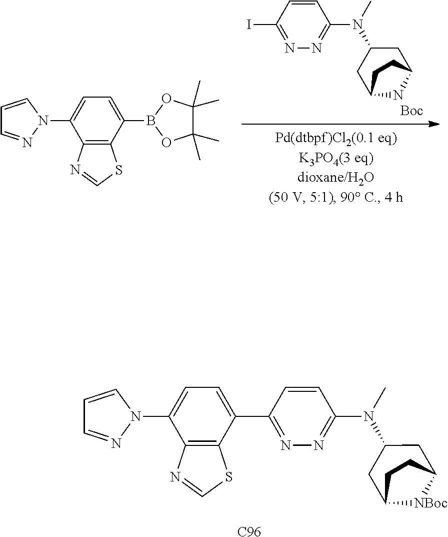

A mixture of 4-(pyrazol-1-yl)-7-(4,4,5,5-tetramethyl-1,3,2-dioxaborolan-2-yl)-1,3-benzothiazole (110.00 mg, 0.3 mmol, 2.99 eq.), tert-butyl (exo)-3-[(6-iodopyridazin-3-yl) (methyl)amino]-8-azabicyclo [3.2.1] octane-8-carboxylate (50.00 mg, 0.1 mmol, 1.00 eq.), Pd(DtBPF)Cl₂ (7.33 mg, 0.011 mmol, 0.10 eq.) and tripotassium phosphate (71.66 mg, 0.3 mmol, 3.00 eq.) in dioxane (3.0 mL) and water (0.6 mL) was stirred for 4 hr at 90° C. under a nitrogen atmosphere. The mixture was allowed to cool down to 25° C. The resulting mixture was diluted with water (50.0 mL). The resulting mixture was extracted with ethyl acetate (2×50.0 mL), dried over anhydrous sodium sulfate. After filtration, the filtrate was concentrated under reduced pressure. The residue was purified by silica gel column chromatography, eluted with dichloromethane/methanol (10:1) to afford tert-butyl (exo)-3-[methyl({6-[4-(pyrazol-1-yl)-1,3-benzothiazol-7-yl] pyridazin-3-yl}) amino]-8-azabicyclo [3.2.1] octane-8-carboxylate (63 mg, C96) as a solid that was taken to the next step without further purification. LCMS (ES, m/z): 518 [M+H]⁺

### Synthesis of Compound 390

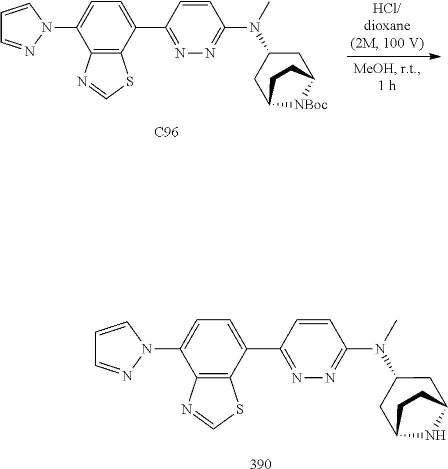

A solution of tert-butyl (exo)-3-[methyl({6-[4-(pyrazol-1-yl)-1,3-benzothiazol-7-yl]pyridazin-3-yl}) amino]-8-azabicyclo [3.2.1] octane-8-carboxylate (63.00 mg, 0.1 mmol, 1.00 eq.) in hydrochloric acid (gas)in 1,4-dioxane (0.7 mL) and methanol (2.1 mL) was stirred for 1 hr at room temperature. The resulting mixture was concentrated under vacuum. The crude product was purified by preparative chiral HPLC (Condition X, Gradient X) to afford (exo)-N-methyl-N-{6-[4-(pyrazol-1-yl)-1,3-benzothiazol-7-yl] pyridazin-3-yl}-8-azabicyclo [3.2.1] octan-3-amine (11 mg, compound 390) as a solid. LCMS (ES, m/z): 418 [M+H]+¹H NMR (400 MHz, DMSO-d₆) δ 9.62 (s, 1H), 9.18 (d, J=2.4 Hz, 1H), 8.31-8.17 (m, 3H), 7.89 (d, J=1.7 Hz, 1H), 7.30 (d, J=9.7 Hz, 1H), 6.65 (dd, J=2.6, 1.8 Hz, 1H), 5.02 (s, 1H), 3.56 (s, 2H), 2.99 (s, 3H), 1.9-1.8 (m, 2H), 1.83 (d, J=21.1 Hz, 4H), 1.58 (d, J=12.4 Hz, 2H).

### Example 116: Synthesis of Compound 391

### Synthesis of Intermediate C97

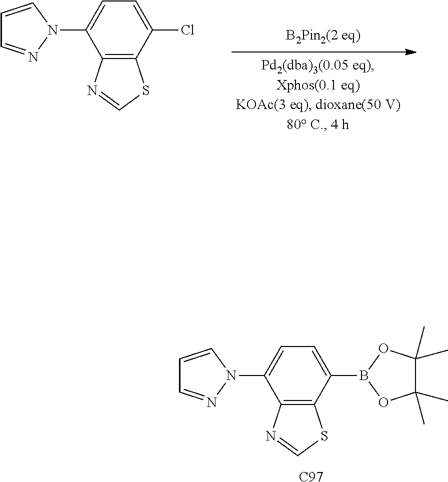 [SMILES: Clc1ccc(-n2cccn2)c2ncsc12 ; CC1(C)OB(c2ccc(-n3cccn3)c3ncsc23)OC1(C)C ; "C97"]

A mixture of 7-chloro-4-(pyrazol-1-yl)-1,3-benzothiazole (200.00 mg, 0.8 mmol, 1.00 equiv), bis(pinacolato)diboron (430.97 mg, 1.6 mmol, 2.00 eq.), Pd₂(dba)₃ (38.85 mg, 0.1 mmol, 0.05 eq.), XPhos (40.45 mg, 0.1 mmol, 0.10 eq.) and potassium aceate (249.84 mg, 2.5 mmol, 3.00 eq.) in 1,4-dioxane (10.0 mL) was stirred for 4 hr at 80° C. under a nitrogen atmosphere. The mixture was allowed to cool down to 25° C. The resulting mixture was diluted with water (50.0 mL). The resulting mixture was extracted with ethyl acetate (2×50.0 mL), dried over anhydrous sodium sulfate. After filtration, the filtrate was concentrated under reduced pressure to afford crude 4-(pyrazol-1-yl)-7-(4,4,5,5-tetramethyl-1,3,2-dioxaborolan-2-yl)-1,3-benzothiazole (250.00 mg, C97) as an oil that was taken to the next step without further purification. LCMS (ES, m/z): 328 [M+H]⁺

### Synthesis of Intermediate C98

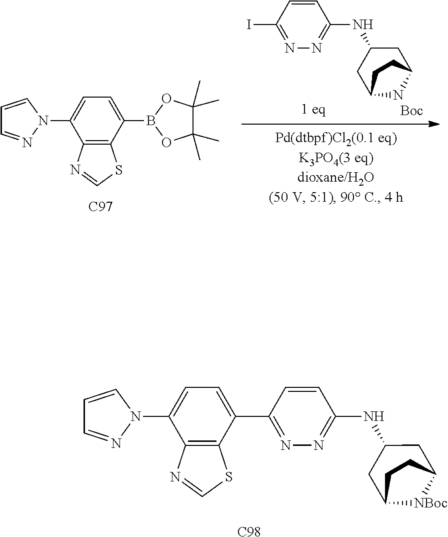

A mixture of 4-(pyrazol-1-yl)-7-(4,4,5,5-tetramethyl-1,3,2-dioxaborolan-2-yl)-1,3-benzothiazole (110.00 mg, 0.3 mmol, 2.89 eq.), tert-butyl (exo)-3-[(6-iodopyridazin-3-yl) amino]-8-azabicyclo [3.2.1] octane-8-carboxylate (50.00 mg, 0.1 mmol, 1.00 eq.), Pd(DtBPF)Cl₂ (20.72 mg, 0.03 mmol, 0.27 eq.) and tripotassium phosphate (202.39 mg, 0.9 mmol, 8.21 eq.) in 1,4-dioxane (3.0 mL) and water (0.6 mL) was stirred for 4 hr at 90° C. under a nitrogen atmosphere. The mixture was allowed to cool down to 25° C. The resulting mixture was diluted with water (50.0 mL). The resulting mixture was extracted with ethyl acetate (2×50.0 mL) and dried over anhydrous sodium sulfate. After filtration, the filtrate was concentrated under reduced pressure. The residue was purified by silica gel column chromatography, eluted with dichloromethane/methanol (10:1) to afford tert-butyl (exo)-3-({6-[4-(pyrazol-1-yl)-1,3-benzothiazol-7-yl] pyridazin-3-yl} amino)-8-azabicyclo [3.2.1] octane-8-carboxylate (53 mg, C98) as a solid that was taken to the next step without further purification. LCMS (ES, m/z): 504 [M+H]⁺

### Synthesis of Compound 391

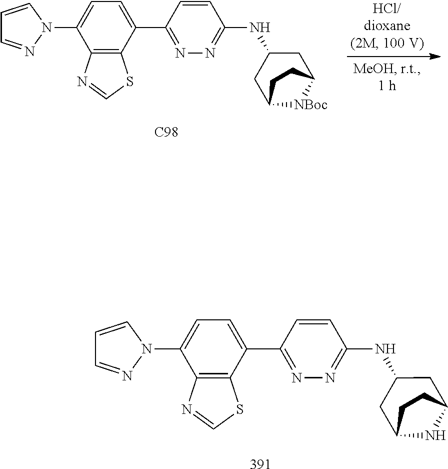

A solution of tert-butyl (exo)-3-({6-[4-(pyrazol-1-yl)-1,3-benzothiazol-7-yl] pyridazin-3-yl}amino)-8-azabicyclo [3.2.1] octane-8-carboxylate (53 mg, 0.1 mmol, 1.00 eq.) in hydrochloric acid (gas)in 1,4-dioxane (0.5 mL) and methanol (1.5 mL) was stirred for 1 hr at room temperature. The resulting mixture was concentrated under vacuum. The crude product was purified by preparative HPLC (Condition 7, Gradient 3) to afford (exo)-N-{6-[4-(pyrazol-1-yl)-1,3-benzothiazol-7-yl] pyridazin-3-yl}-8-azabicyclo [3.2.1] octan-3-amine (11.20 mg, Compound 391) as a solid. LCMS (ES, m/z): 404 [M+H]⁺¹H NMR (400 MHz, DMSO-d₆) δ 9.61 (s, 1H), 9.18 (d, J=2.4 Hz, 1H), 8.20 (s, 2H), 8.16 (d, J=9.5 Hz, 1H), 7.88 (d, J=1.7 Hz, 1H), 6.99 (dd, J=16.5, 8.5 Hz, 2H), 6.77-6.43 (m, 1H), 4.38 (m, 2H), 3.51 (s, 2H), 1.99 (d, J=12.7 Hz, 2H), 1.77 (m, 4H), 1.46 (t, J=11.8 Hz, 2H).

### Example 117: Synthesis of Compound 392

### Synthesis of Intermediate C99

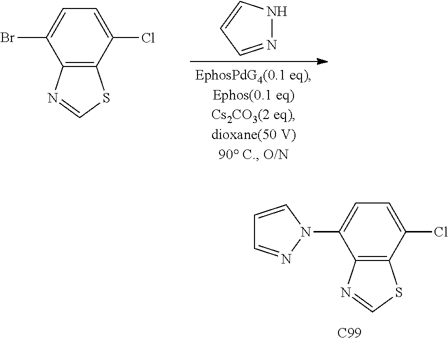 [SMILES: Clc1ccc(Br)c2ncsc12 ; c1cn[nH]c1 ; Clc1ccc(-n2cccn2)c2ncsc12 ; "C99"]

A solution of 4-bromo-7-chloro-1,3-benzothiazole (500 mg, 1.972 mmol, 1.00 eq.), pyrazole (134.23 mg, 1.972 mmol, 1 eq.), EPhos Pd G₄ (181.11 mg, 0.197 mmol, 0.1 eq.), EPhos (105.44 mg, 0.197 mmol, 0.1 eq.) and cesium carbonate (300.25 mg, 3.944 mmol, 2 eq.) in 1,4-dioxane (10 mL) was stirred for overnight at 90° C. under nitrogen atmosphere. The mixture was allowed to cool down to room temperature. The resulting mixture was diluted with water (100 mL). The resulting mixture was extracted with ethyl acetate (3×100 mL), dried over anhydrous sodium sulfate. After filtration, the filtrate was concentrated under reduced pressure. The residue was purified by silica gel column chromatography, eluted with petroleum ether/ethyl acetate (5:1) to afford 7-chloro-4-(pyrazol-1-yl)-1,3-benzothiazole (270 mg, C99) as a solid that was taken to the next step without further purification. LCMS (ES, m/z): 236 [M+H]⁺

### Synthesis of Intermediate C100

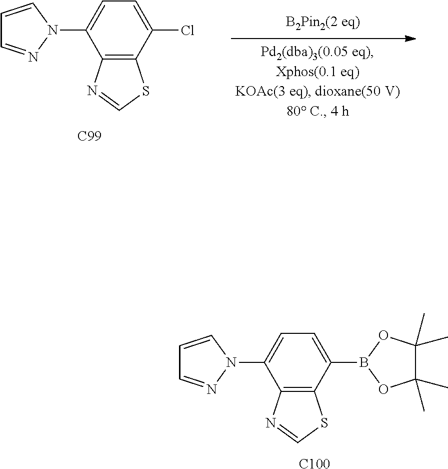 [SMILES: Clc1ccc(-n2cccn2)c2ncsc12 ; "C99" ; CC1(C)OB(c2ccc(-n3cccn3)c3ncsc23)OC1(C)C ; "C100"]

A solution of 7-chloro-4-(pyrazol-1-yl)-1,3-benzothiazole (270 mg, 1.088 mmol, 1.00 eq.), bis(pinacolato)diboron (552.72 mg, 2.176 mmol, 2 eq.), XPhos (51.88 mg, 0.109 mmol, 0.1 eq.), Pd2(dba)₃ CHCl₃ (56.32 mg, 0.054 mmol, 0.05 eq.) and potassium acetate (213.62 mg, 2.176 mmol, 2 eq.) in 1,4-dioxane (10 mL, 113.501 mmol, 104.29 eq.) was stirred for 4 hr at 80° C. under a nitrogen atmosphere. The mixture was allowed to cool down to room temperature. The resulting mixture was diluted with water (100 mL). The resulting mixture was extracted with ethyl acetate (3×100 mL), dried over anhydrous sodium sulfate. After filtration, the filtrate was concentrated under reduced pressure. The crude product was used in the next step directly without further purification. LCMS (ES, m/z): 328[M+H]⁺

### Synthesis of Intermediate C101

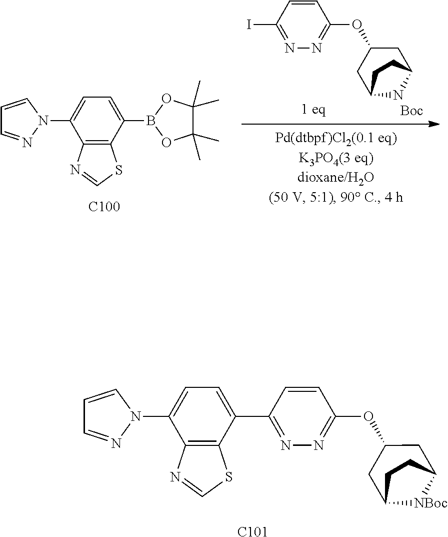

A solution of 4-(pyrazol-1-yl)-7-(4,4,5,5-tetramethyl-1,3,2-dioxaborolan-2-yl)-1,3-benzothiazole (60 mg, 0.183 mmol, 1.00 eq.), tert-butyl (exo)-3-[(6-iodopyridazin-3-yl)oxy]-8-azabicyclo[3.2.1]octane-8-carboxylate (79.08 mg, 0.183 mmol, 1 eq.), Pd(DtBPF)Cl₂ (11.95 mg, 0.018 mmol, 0.1 eq.) and tripotassium phosphate (116.77 mg, 0.549 mmol, 3 eq.) in 1,4-dioxane (3 mL) and water (0.6 mL) was stirred for 4 hr at 90° C. under a nitrogen atmosphere. The mixture was allowed to cool down to room temperature. The resulting mixture was diluted with water (30 mL). The resulting mixture was extracted with ethyl acetate (3×30 mL), dried over anhydrous sodium sulfate. After filtration, the filtrate was concentrated under reduced pressure. The residue was purified by silica gel column chromatography, eluted with petroleum ether/ethyl acetate (1:1) to afford tert-butyl (exo)-3-({6-[4-(pyrazol-1-yl)-1,3-benzothiazol-7-yl]pyridazin-3-yl}oxy)-8-azabicyclo[3.2.1]octane-8-carboxylate (60 mg, C101) as a solid that was taken to next step without further purification. LCMS (ES, m/z): 505[M+H]⁺

### Synthesis of Compound 392

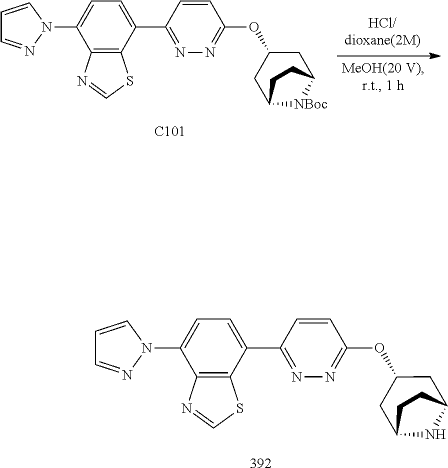

A solution of tert-butyl (exo)-3-({6-[4-(pyrazol-1-yl)-1,3-benzothiazol-7-yl]pyridazin-3-yl}oxy)-8-azabicyclo[3.2.1]octane-8-carboxylate (60 mg, 0.119 mmol, 1.00 eq.) in hydrochloric acid (gas)in 1,4-dioxane (1.8 mL) and methanol (1.2 mL) was stirred for 1 hr at room temperature. The resulting mixture was concentrated under reduced pressure. The crude product was purified by preparative HPLC (Condition 7, Gradient 7) to afford 7-{6-[(exo)-8-azabicyclo[3.2.1]octan-3-yloxy]pyridazin-3-yl}-4-(pyrazol-1-yl)-1,3-benzothiazole (28.3 mg, Compound 392) as a solid. LCMS (ES, m/z): 405[M+H]⁺¹H NMR (400 MHz, DMSO-d₆) δ 9.65 (s, 1H), 9.23 (d, J=2.5 Hz, 1H), 8.54 (d, J=9.4 Hz, 1H), 8.40 (d, J=8.4 Hz, 1H), 8.25 (d, J=8.3 Hz, 1H), 7.91 (d, J=1.7 Hz, 1H), 7.37 (d, J=9.4 Hz, 1H), 6.67 (t, J=2.2 Hz, 1H), 5.62 (tt, J=11.0, 5.9 Hz, 1H), 3.53 (s, 2H), 2.20 (ddd, J=12.4, 6.0, 2.9 Hz, 3H), 1.78-1.73 (m, 4H), 1.63 (td, J=11.5, 2.9 Hz, 2H).

### Example 118: Synthesis of Compound 393

### Synthesis of Intermediate C102

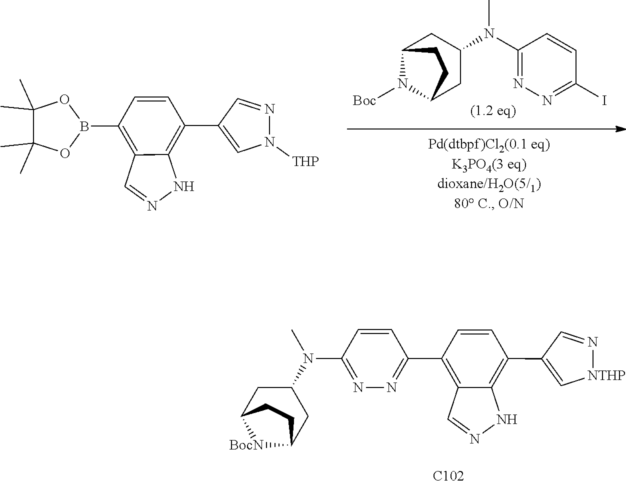

Into a 40-mL vial, was placed 7-(1-(tetrahydro-2H-pyran-2-yl)-1H-pyrazol-4-yl)-4-(4,4,5,5-tetrameth-yl-1,3,2-dioxaborolan-2-yl)-1H-indazole (50.0 mg, 0.12 mmol, 1.00 eq.), tert-butyl (1R,3s,5S)-3-((6-iodopyridazin-3-yl)(methyl)amino)-8-azabicyclo[3.2.1]octane-8-carboxylate (67.6 mg, 0.15 mmol, 1.20 eq.), dioxane (3.00 mL), tripotassium phosphate (80.7 mg, 0.36 mmol, 3.00 eq.), water (0.6 mL), Pd(dtbpf)Cl₂ (8.2 mg, 0.012 mmol, 0.10 eq.). The reaction mixture was evacuated and flushed three times with nitrogen. The resulting solution was stirred for 16 hr at 80° C. The reaction was then quenched by the addition of 20 mL of water. The resulting solution was extracted with 3×30 mL of ethyl acetate and the organic layers combined. The resulting mixture was washed with 1×50 ml of saturated aqueous sodium chloride. The mixture was dried over anhydrous sodium sulfate. The solids were filtered out. The resulting mixture was concentrated under vacuum. The residue was applied onto a silica gel column with ethyl acetate/petroleum ether to afford 40 mg (54%) of tert-butyl (1R,3s,5S)-3-(methyl(6-(7-(1-(tetrahydro-2H-pyran-2-yl)-1H-pyrazol-4-yl)-1H-indazol-4-yl)pyridazin-3-yl)amino)-8-azabicyclo[3.2.1]octane-8-carboxylate as a solid that was taken to the next step without further purification. LCMS (ES, m/z): 585 [M+H]⁺

### Synthesis of Compound 393

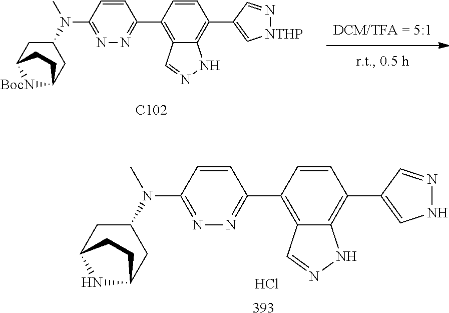

Into a 25-mL round-bottom flask, was tert-butyl (1R,3s,5S)-3-(methyl(6-(7-(1-(tetrahydro-2H-pyran-2-yl)-1H-pyrazol-4-yl)-1H-indazol-4-yl)pyridazin-3-yl)amino)-8-azabicyclo[3.2.1]octane-8-carboxylate (40.0 mg, 0.07 mmol, 1.00 eq.), dichloromethane (2.0 mL), and trifluoroacetic acid (0.40 mL). The resulting mixture was stirred for 30 min at room temperature. The resulting mixture was concentrated under vacuum. The crude product was purified by preparative HPLC (Condition 8, Gradient 2) to afford (1R,3s,5S)-N-(6-(7-(1H-pyrazol-4-yl)-1H-indazol-4-yl)pyridazin-3-yl)-N-methyl-8-azabicyclo [3.2.1]octan-3-amine (12 mg, Compound 393) as a solid. LCMS (ES, m/z): 401 [M−HCl]⁺ ¹H-NMR (400 MHz, DMSO-d₆) δ 13.36 (s, 1H), 9.77 (s, 1H), 8.98 (s, 1H), 8.73 (s, 1H), 8.57 (d, J=10.0 Hz, 1H), 8.42 (s, 2H), 8.10 (s, 1H), 7.87 (d, J=7.7 Hz, 1H), 7.76 (d, J=7.5 Hz, 1H), 4.77 (s, 1H), 4.13 (s, 2H), 3.24 (s, 3H), 2.40 (m, 2H), 2.25 (d, J=8.4 Hz, 2H), 2.02 (dd, J=9.1, 4.4 Hz, 2H), 1.91 (dd, J=13.8, 5.6 Hz, 2H).

### Example 119: Synthesis of Compound 394

### Synthesis of Intermediate C103

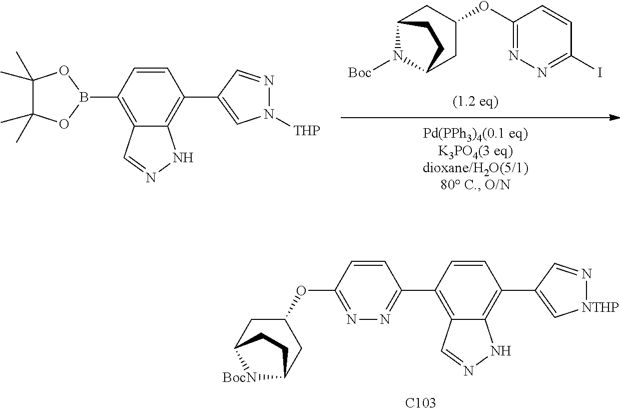

Into a 40-mL vial, was placed 7-(1-(tetrahydro-2H-pyran-2-yl)-1H-pyrazol-4-yl)-4-(4,4,5,5-tetrameth-yl-1,3,2-dioxaborolan-2-yl)-1H-indazole (50.0 mg, 0.12 mmol, 1.00 eq.), tert-butyl (1R,3s,5S)-3-((6-iodopyridazin-3-yl)oxy)-8-azabicyclo[3.2.1]octane-8-carboxylate (65.6 mg, 0.15 mmol, 1.20 eq.), dioxane (3.00 mL), tripotassium phosphate (80.7 mg, 0.36 mmol, 3.00 eq.), water (0.6 mL), Pd(PPh₃)₄(14.6 mg, 0.012 mmol, 0.10 eq.). The reaction mixture was evacuated and flushed three times with nitrogen. The resulting solution was stirred for 16 hr at 80° C. The reaction was then quenched by the addition of 20 mL of water. The resulting solution was extracted with 3×30 mL of ethyl acetate and the organic layers were combined. The resulting mixture was washed with 1×50 ml of saturated aqueous sodium chloride. The mixture was dried over anhydrous sodium sulfate. The solids were filtered out. The resulting mixture was concentrated under vacuum. The residue was applied onto a silica gel column with ethyl acetate/petroleum ether to afford 40 mg (55%) of tert-butyl (1R,3s,5S)-3-((6-(7-(1-(tetrahydro-2H-pyran-2-yl)-1H-pyrazol-4-yl)-1H-indazol-4-yl)pyridazin-3-yl)oxy)-8-azabicyclo[3.2.1]octane-8-carboxylate as a solid that was taken to the next step without further purification. LCMS (ES, m/z): 572 [M+H]⁺

### Synthesis of Compound 394

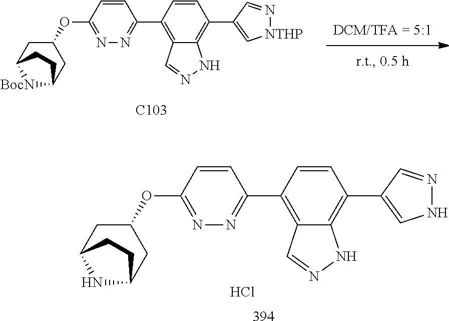

Into a 25-mL round-bottom flask, was placed tert-butyl (1R,3s,5S)-3-((6-(7-(1-(tetrahydro-2H-pyran-2-yl)-1H-pyrazol-4-yl)-1H-indazol-4-yl)pyridazin-3-yl)oxy)-8-azabicyclo[3.2.1]octane-8-carboxylate (40.0 mg, 0.07 mmol, 1.00 eq.), dichloromethane (2.0 mL), and trifluoroacetic acid (0.40 mL). The resulting mixture was stirred for 30 min at room temperature. The resulting mixture was concentrated under vacuum. The crude product was purified by preparative HPLC (Condition 8, Gradient 2). This resulted in 6.7 mg (24%) of 4-(6-(((1R,3s,5S)-8-azabicyclo[3.2.1]octan-3-yl)oxy)pyridazin-3-yl)-7-(1H-pyrazol-4-yl)-1H-indazole as solid (Compound 394). LCMS (ES, m/z): 388 [M+H]⁺¹H-NMR (400 MHz, DMSO-d₆) δ 13.26 (s, 1H), 8.91 (d, J=19.7 Hz, 2H), 8.70 (s, 1H), 8.38 (s, 2H), 8.32 (d, J=9.4 Hz, 1H), 7.73 (s, 2H), 7.35 (d, J=9.3 Hz, 1H), 5.68 (tt, J=11.1, 5.9 Hz, 1H), 4.16 (s, 2H), 2.53 (s, 2H), 2.14-2.05 (m, 4H), 2.05-1.93 (m, 2H).

### Example 120: Synthesis of Compound 395

### Synthesis of Intermediate C104

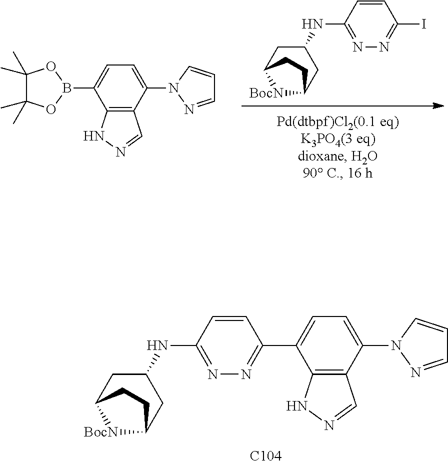

To a solution of 4-(pyrazol-1-yl)-7-(4,4,5,5-tetramethyl-1,3,2-dioxaborolan-2-yl)-1H-indazole (100 mg, 0.322 mmol, 1.00 eq.) and tert-butyl(exo)-3-[(6-iodopyridazin-3-yl)amino]8-azabicyclo(3.2.1)octane-8-carboxylate(134.86 mg, 0.322 mmol, 1 eq.) in dioxane (2 mL, 23.608 mmol, 73.22 eq.) and water (0.4 mL, 22.203 mmol, 68.87 eq.) were added Pd(dtbpf)Cl₂ (21.01 mg, 0.032 mmol, 0.1 eq.) and tripotassium phosphate (205.31 mg, 0.966 mmol, 3 eq.). After stirring for 2 hr at 80° C. under a nitrogen atmosphere, the resulting mixture was concentrated under reduced pressure. The residue was purified by silica gel column chromatography, eluted with dichloromethane/methanol (10:01) to afford tert-butyl (exo)-3-[(6-[4-(1H-pyrazol-1-yl)-1H-indazol-7-yl]pyridazin-3-yl)amino]8-azabicyclo[3.2.1]octane-8-carboxylate (100 mg, C104) as a solid that was taken to the next step without further purification. LCMS (ES, m/z): 487 [M+H]+

### Synthesis of Compound 395

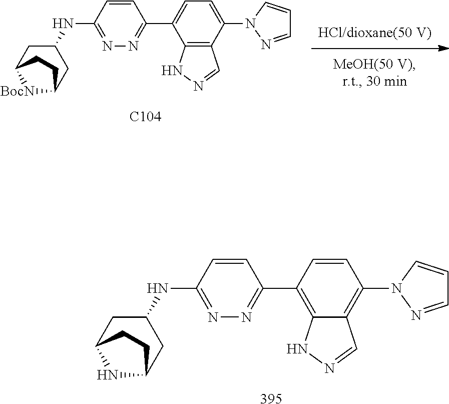

Into a 10 mL round-bottom flask were added tert-butyl (exo)-3-[(6-[4-(1H-pyrazol-1-yl)-1H-indazol-7-yl]pyridazin-3-yl)amino]8-azabicyclo[3.2.1]octane-8-carboxylate (79 mg, 0.166 mmol, 1.00 eq.) and hydrochloric acid (gas)in 1,4-dioxane (0.5 mL, 16.456 mmol, 98.85 eq.) at room temperature. The resulting mixture was stirred for 30 min at room temperature. The resulting mixture was concentrated under reduced pressure. The residue was purified by reverse flash chromatography. This resulted in 7-(6-((exo)-8-azabicyclo[3.2.1]octan-3-yl)amino)pyridazin-3-yl)-1H-indazole (22.6 mg, Compound 395) as a solid. LCMS (ES, m/z): 386 [M+H]⁺¹H-NMR (400 MHz, DMSO-d₆): δ 13.24 (s, 1H), 8.67 (d, J=2.5 Hz, 1H), 8.61 (s, 1H), 8.07 (d, J=9.6 Hz, 1H), 7.97-7.89 (m, 2H), 7.59 (d, J=8.0 Hz, 1H), 6.96 (d, J=9.5 Hz, 1H), 6.90 (d, J=9.5 Hz, 1H), 6.65 (dd, J=2.5, 1.7 Hz, 1H), 4.37 (tt, J=11.3, 5.5 Hz, 1H), 3.46 (s, 2H), 2.3-2.0 (s, 1H), 1.97 (dt, J=14.6, 3.6 Hz, 2H), 1.73 (d, J=2.2 Hz, 4H), 1.46 (td, J=12.1, 2.8 Hz, 2H).

### Example 121: Synthesis of Compound 396

### Synthesis of Intermediate C105

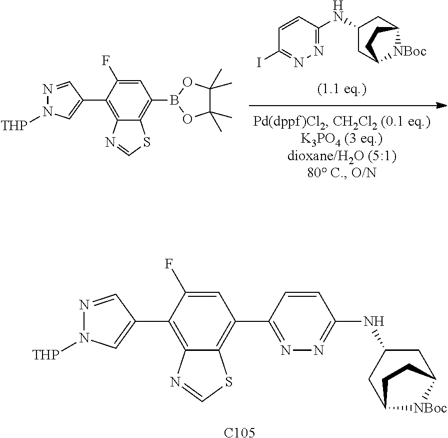

To a solution of 5-fluoro-4-[1-(oxan-2-yl) pyrazol-4-yl]-7-(4,4,5,5-tetramethyl-1,3,2-dioxaborolan-2-yl)-1,3-benzothiazole (70.00 mg, 0.16 mmol, 1.00 eq.) and tert-butyl (1R,3R,5S)-3-[(6-iodopyridazin-3-yl) amino]-8-azabicyclo [3.2.1] octane-8-carboxylate (77.17 mg, 0.18 mmol, 1.10 eq.) in dioxane (2.00 mL) and water (0.50 mL) were added tripotassium phosphate (103.83 mg, 0.49 mmol, 3.00 eq.) and Pd(dppf)Cl₂·CH₂Cl₂ (13.28 mg, 0.01 mmol, 0.10 eq.). After stirring overnight at 80° C. under a nitrogen atmosphere, the resulting mixture was concentrated under reduced pressure. The residue was purified by preparative thin layer chromatography/silica gel column chromatography, eluted with petroleum ether/ethyl acetate (5:1) to afford tert-butyl (1R,3R,5S)-3-[(6-{5-fluoro-4-[1-(oxan-2-yl) pyrazol-4-yl]-1,3-benzothiazol-7-yl} pyridazin-3-yl) amino]-8-azabicyclo [3.2.1] octane-8-carboxylate (70 mg, C105) as a solid. LCMS (ES, m/z):606 [M+H]⁺

### Synthesis of Compound 396

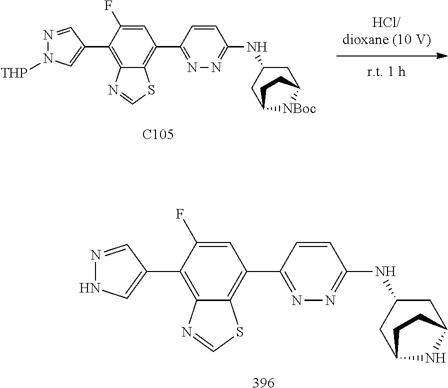

Into an 8 mL vial were added tert-butyl (1R,3R,5S)-3-[(6-{5-fluoro-4-[1-(oxan-2-yl) pyrazol-4-yl]-1,3-benzothiazol-7-yl} pyridazin-3-yl) amino]-8-azabicyclo [3.2.1] octane-8-carboxylate (60.00 mg, 0.099 mmol, 1.00 eq.) and hydrochloric acid (gas)in 1,4-dioxane (1 mL) at room temperature. The resulting mixture was stirred for 1 hr at room temperature. The residue was purified by reverse flash chromatography (Condition 3, Gradient 2). This resulted in (1R,3R,5S)-N-{6-[5-fluoro-4-(1H-pyrazol-4-yl)-1,3-benzothiazol-7-yl] pyridazin-3-yl}-8-azabicyclo [3.2.1] octan-3-amine (15.7 mg, Compound 396) as a solid. LCMS (ES, m/z):422 [M+H]+¹H-NMR (400 MHz, DMSO-d₆) δ 13.23 (s, 1H), 9.58 (s, 1H), 8.49 (s, 2H), 8.18 (d, J=9.6 Hz, 1H), 8.10 (d, J=13.0 Hz, 1H), 7.04 (d, J=7.8 Hz, 1H), 6.95 (d, J=9.5 Hz, 1H), 4.38 (d, J=9.7 Hz, 1H), 3.53 (s, 2H), 1.99 (d, J=12.7 Hz, 2H), 1.78 (s, 4H), 1.46 (t, J=11.6 Hz, 2H).

### Example 122: Synthesis of Compound 397

### Synthesis of Intermediate C106

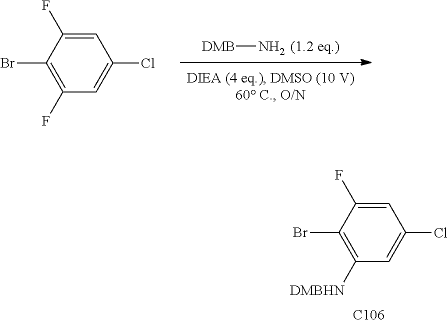

A mixture of 2-bromo-5-chloro-1,3-difluorobenzene (90.00 g, 395.73 mmol, 1.00 equiv), DIEA (204.58 g, 1582.90 mmol, 4.00 eq.) and 1-(3,4-dimethylphenyl) methanamine (64.21 g, 474.87 mmol, 1.20 eq.) in dimethyl sulfoxide (900.00 mL) was stirred overnight at 60° C. under a nitrogen atmosphere. The resulting mixture was extracted with ethyl acetate (3×1000 mL). The combined organic layers were washed with water (2×1000 mL), and dried over anhydrous sodium sulfate. After filtration, the filtrate was concentrated under reduced pressure. The residue was purified by silica gel column chromatography, eluted with petroleum ether/ethyl acetate (10:1) to afford 2-bromo-5-chloro-N-[(2,4-dimethoxyphenyl) methyl]-3-fluoroaniline (80.0 g, C106) as a solid. LCMS (ES, m/z):374 [M+H] ⁺¹H-NMR (400 MHz, DMSO-d₆) δ 7.09 (d, J=8.4 Hz, 1H), 6.67 (dt, J=8.6, 2.0 Hz, 1H), 6.59 (d, J=2.4 Hz, 1H), 6.48 (dd, J=8.4, 2.4 Hz, 1H), 6.42 (t, J=1.9 Hz, 1H), 6.25 (t, J=6.1 Hz, 1H), 4.30 (d, J=6.1 Hz, 2H), 3.85 (s, 3H), 3.74 (s, 3H).

### Synthesis of Intermediate C107

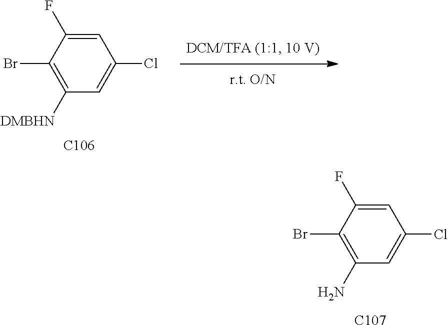

A mixture of 2-bromo-5-chloro-N-[(2,4-dimethoxyphenyl) methyl]-3-fluoroaniline (70.00 g, 186.85 mmol, 1.00 eq.), trifluoroacetic acid (350.00 mL) in dichloromethane (350.00 mL) was stirred overnight at room temperature under a nitrogen atmosphere. The mixture/residue was neutralized to pH 7 with saturated aqueous sodium carbonate. The resulting mixture was extracted with dichloromethane (3×1000 mL) and dried over anhydrous sodium sulfate. After filtration, the filtrate was concentrated under reduced pressure. The residue was purified by silica gel column chromatography, eluted with petroleum ether/ethyl acetate (10:1) to afford 2-bromo-5-chloro-3-fluoroaniline (28 g, C107) as a solid that was taken to the next step without further purification. LCMS (ES, m/z):224 [M+H]⁺¹H-NMR (400 MHz, Chloroform-d) δ 6.56 (d, J=7.8 Hz, 2H), 4.33 (s, 2H).

### Synthesis of Intermediate C108

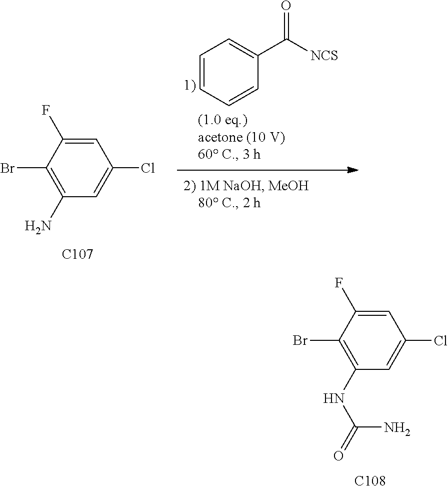

Benzoyl isothiocyanate (18.90 g, 115.83 mmol, 1.00 eq.) was added dropwise to a stirred solution of 2-bromo-5-chloro-3-fluoroaniline (26.0 g, 115.83 mmol, 1.00 eq.) in acetone (300.00 mL). The reaction mixture was heated to reflux for 3 hr. Then poured into ice water and stirred for an additional 30 min. The precipitate was collected by filtration and washed with more water. This crude material was dissolved in methanol (300.00 mL) and treated with sodium hydroxide (1 M; 75.00 mL). The reaction mixture was heated to 80° C. for 2 hr. After cooling, the reaction mixture was poured into water-ice and sufficient aqueous 1M hydrochloric acid was added to produce a neutral (pH˜ 7) solution. The precipitates from the neutral solution and was collected by filtration and dried to afford 2-bromo-5-chloro-3-fluorophenylurea (26 g, C108). LCMS (ES, m/z): 283 [M+H]⁺

### Synthesis of Intermediate C109

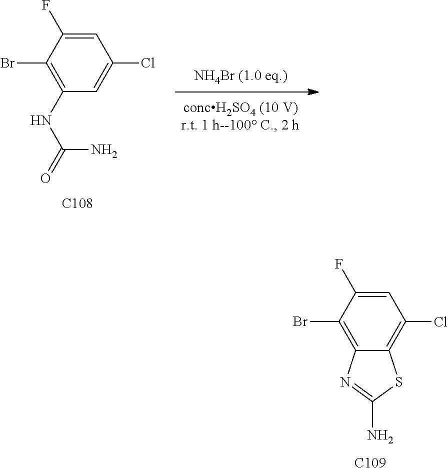 [SMILES: NC(=O)Nc1cc(Cl)cc(F)c1Br ; "C108" ; Nc1nc2c(Br)c(F)cc(Cl)c2s1 ; "C109"]

To a stirred solution of 2-bromo-5-chloro-3-fluorophenylurea (26.00 g, 97.20 mmol, 1.00 eq.) in concentrated sulfuric acid (300.00 mL) was added amine hydrobromide (9.52 g, 97.20 mmol, 1.00 eq.) over 1 hr, and then the reaction mixture was heated at 100° C. for 2 hr. The reaction mixture was cooled to room temperature and then poured into ice water (150 ml), at which point a white precipitate was observed. The pH was adjusted to 7 using aqueous ammonium hydroxide solution. The solid was collected by filtration, washed with water and dried in vacuum to afford 4-bromo-7-chloro-5-fluoro-1,3-benzothiazol-2-amine (22.6 g, C109). LCMS (ES, m/z):281 [M+H] ⁺¹H-NMR (400 MHz, DMSO-d₆) δ 8.29 (s, 2H), 7.29 (d, J=9.1 Hz, 1H).

### Synthesis of Intermediate C110

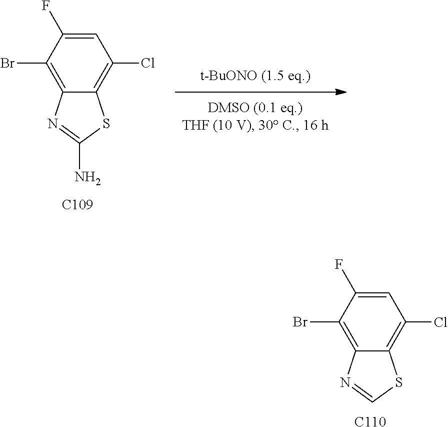 [SMILES: Nc1nc2c(Br)c(F)cc(Cl)c2s1 ; "C109" ; Fc1cc(Cl)c2scnc2c1Br ; "C110"]

A solution of 4-bromo-7-chloro-5-fluoro-1,3-benzothiazol-2-amine (22.6 g, 80.27 mmol, 1.00 eq.) in tetrahydrofuran (90.00 mL) was added dropwise over 20 min to a solution of t-BuONO (12.42 g, 120.41 mmol, 1.50 eq.) and dimethylsulfoxide (0.63 g, 8.03 mmol, 0.10 eq.) in tetrahydrofuran (210.00 mL) at 30° C. The mixture was stirred at 30° C. for 16 hr. Thin layer chromatography showed the reaction was completed. The mixture was diluted with ethyl acetate (1000 mL) and water (1000 mL), and the aqueous layer was extracted with ethyl acetate (2×500 mL). The organic layers were combined and washed with saturated brine (500 mL), dried over anhydrous sodium sulfate and concentrated to give a residue. The residue was purified by flash column chromatography (80 g silica gel column, 20% ethyl acetate in petroleum ether) to afford 4-bromo-7-chloro-5-fluoro-1,3-benzothiazole (19.4 g, C110) as a solid. LCMS (ES, m/z):266 [M+H]+¹H-NMR (400 MHz, DMSO-d₆) δ 9.65 (s, 1H), 7.91 (d, J=8.9 Hz, 1H).

### Synthesis of Intermediate C110

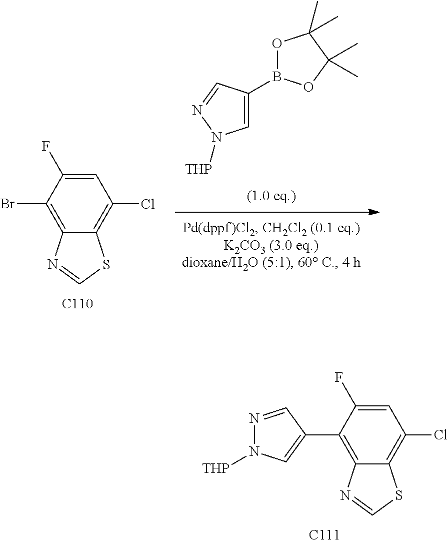

To a solution of 4-bromo-7-chloro-5-fluoro-1,3-benzothiazole (1.00 g, 3.75 mmol, 1.00 eq.) and 1-(oxan-2-yl)-4-(4,4,5,5-tetramethyl-1,3,2-dioxaborolan-2-yl) pyrazole (1.04 g, 3.75 mmol, 1.00 eq.) in water (2.00 mL) and dioxane (10.00 mL) were added potassium carbonate (1.56 g, 11.25 mmol, 3.00 eq.) and Pd(dppf)Cl₂·CH₂Cl₂ (0.31 g, 0.37 mmol, 0.10 eq.). After stirring for 4 hr at 60° C. under a nitrogen atmosphere, the resulting mixture was concentrated under reduced pressure. The residue was purified by preparative thin layer chromatography/silica gel column chromatography, eluted with petroleum ether/ethyl acetate (8:1) to afford 7-chloro-5-fluoro-4-[1-(oxan-2-yl) pyrazol-4-yl]-1,3-benzothiazole (700 mg, C111) as a solid. LCMS (ES, m/z):338 [M+H]⁺

### Synthesis of Intermediate C112

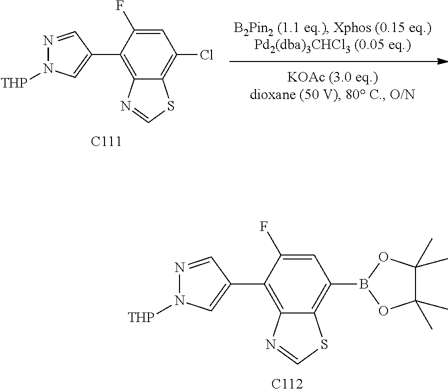

A mixture of 7-chloro-5-fluoro-4-[1-(oxan-2-yl) pyrazol-4-yl]-1,3-benzothiazole (500.00 mg, 1.48 mmol, 1.00 eq.) and Pd₂(dba)₃CHCl₃(76.61 mg, 0.07 mmol, 0.05 eq.) in dioxane (15.00 mL) was stirred overnight at 80° C. under a nitrogen atmosphere. The precipitated solids were collected by filtration and washed with 1,4-dioxane (2×10 mL). This resulted in 5-fluoro-4-[1-(oxan-2-yl) pyrazol-4-yl]-7-(4,4,5,5-tetramethyl-1,3,2-dioxaborolan-2-yl)-1,3-benzothiazole (500.0 mg, C112) as a liquid that was taken to the next step without further purification. LCMS (ES, m/z):430 [M+H]⁺

### Synthesis of Intermediate C113

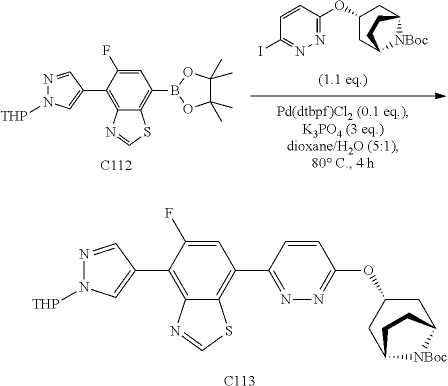

To a solution of 5-fluoro-4-[1-(oxan-2-yl) pyrazol-4-yl]-7-(4,4,5,5-tetramethyl-1,3,2-dioxaborolan-2-yl)-1,3-benzothiazole (70.00 mg, 0.16 mmol, 1.00 eq.) and tert-butyl (1R,3R,5S)-3-[(6-iodopyridazin-3-yl) oxy]-8-azabicyclo [3.2.1] octane-8-carboxylate (77.35 mg, 0.18 mmol, 1.10 eq.) in dioxane (2.00 mL) and water (0.50 mL) were added tripotassium phosphate (103.83 mg, 0.49 mmol, 3.00 eq.) and Pd(dtbpf)Cl₂ (10.63 mg, 0.02 mmol, 0.10 eq.). After stirring for 4 hr at 80° C. under a nitrogen atmosphere, the resulting mixture was concentrated under reduced pressure. The residue was purified by silica gel column chromatography, eluted with petroleum ether/ethyl acetate (5:1) to afford tert-butyl (1R,3R,5S)-3-[(6-{5-fluoro-4-[1-(oxan-2-yl) pyrazol-4-yl]-1,3-benzothiazol-7-yl} pyridazin-3-yl) oxy]-8-azabicyclo [3.2.1] octane-8-carboxylate (60.00 mg, C113) as a solid that was taken to the next step without further purification.

### Synthesis of Compound 397

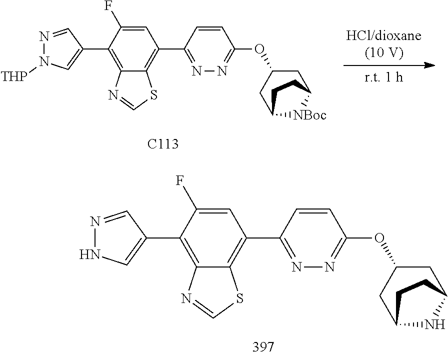

Into an 8 mL vial were added tert-butyl (1R,3R,5S)-3-[(6-{5-fluoro-4-[1-(oxan-2-yl) pyrazol-4-yl]-1,3-benzothiazol-7-yl} pyridazin-3-yl) oxy]-8-azabicyclo [3.2.1] octane-8-carboxylate (50.00 mg, 1.0 eq.) and hydrochloric acid (gas)in 1,4-dioxane (0.5 mL) at room temperature. The resulting mixture was stirred for 1 hr at room temperature. The crude product was purified by preparative chiral HPLC (Condition 3, Gradient 1). This resulted in 7-{6-[(1R,3R,5S)-8-azabicyclo [3.2.1] octan-3-yloxy] pyridazin-3-yl}-5-fluoro-4-(1H-pyrazol-4-yl)-1,3-benzothiazole hydrochloride (4.4 mg, Compound 397) as a solid. LCMS (ES, m/z):423 [M+H]+¹H-NMR (400 MHz, DMSO-d₆) δ 9.63 (s, 1H), 9.05 (d, J=21.1 Hz, 2H), 8.64 (d, J=9.4 Hz, 1H), 8.54 (d, J=2.0 Hz, 2H), 8.36 (d, J=12.8 Hz, 1H), 7.47 (d, J=9.3 Hz, 1H), 5.70 (dt, J=10.7, 5.2 Hz, 1H), 4.14 (s, 2H), 2.48 (m, 2H), 2.21-1.93 (m, 6H).

### Example 123: Synthesis of Compound 402

### Synthesis of Intermediate C114

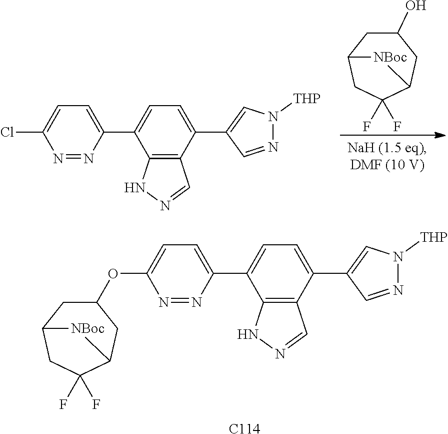

To a solution of tert-butyl 6,6-difluoro-3-hydroxy-8-azabicyclo[3.2.1]octane-8-carboxylate (55 mg, 0.210 mmol, 1 equiv) in DMF was added sodium hydride (60% in oil, 3 mg) at 0° C. The mixture was stirred for 15 min. 7-(6-chloropyridazin-3-yl)-4-[1-(oxan-2-yl)pyrazol-4-yl]-1H-indazole (80 mg, 0.210 mmol, 1.00 equiv) was added and the mixture was allowed to warm to RT and stirred for 2 h. The reaction mixture was quenched by water and extracted with DCM (3×25 mL). The resulting mixture was concentrated under reduced pressure. This resulted in tert-butyl 6,6-difluoro-3-[(6-{4-[1-(oxan-2-yl)pyrazol-4-yl]-1H-indazol-7-yl}pyridazin-3-yl)oxy]-8-azabicyclo[3.2.1]octane-8-carboxylate (80 mg, 62.67%) as an off-white solid. LCMS (ES, m/z): 608 [M+H]⁺

### Synthesis of Compound 402

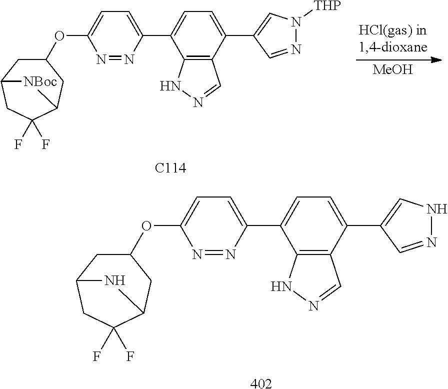

Into a 50 mL round-bottom flask were added tert-butyl 6,6-difluoro-3-[(6-{4-[1-(oxan-2-yl)pyrazol-4-yl]-1H-indazol-7-yl}pyridazin-3-yl)oxy]-8-azabicyclo[3.2.1]octane-8-carboxylate (80 mg, 0.132 mmol, 1.00 equiv) and HCl(gas)in 1,4-dioxane (2 mL), MeOH (2 mL) at room temperature. The resulting mixture was stirred for 30 min at room temperature under nitrogen atmosphere. The resulting mixture was concentrated under reduced pressure. The residue was dissolved in DMF (5 mL). The residue was purified by reverse flash chromatography with the following conditions: Column: YMC-Actus Triart C18, 30×150 mm, 5 μm; Mobile Phase A: water (0.05% HCl), Mobile Phase B: ACN; Flow rate: 60 mL/min; Gradient: 5% B to 33% B in 8 min, 33% B; Wave Length: 220 nm; RT1(min):7.03) to afford 7-[6-({6,6-difluoro-8-azabicyclo[3.2.1]octan-3-yl}oxy)pyridazin-3-yl]-4-(1H-pyrazol-4-yl)-1H-indazole (5.8 mg, Compound 402) as a solid. LCMS (ES, m/z): 424 [M+H]⁺1H NMR (400 MHz, DMSO-d6) δ 13.22 (s, 2H), 8.55 (s, 1H), 8.53 (s, 1H), 8.42 (d, J=9.4 Hz, 1H), 8.22 (s, 1H), 8.00 (d, J=7.8 Hz, 1H), 7.51 (d, J=7.6 Hz, 1H), 7.34 (d, J=9.3 Hz, 1H), 5.75 (tt, J=11.1, 6.4 Hz, 1H), 3.67 (d, J=7.5 Hz, 1H), 3.52 (d, J=13.1 Hz, 1H), 2.98 (s, 1H), 2.45-2.40 (m, 1H), 2.38-2.33 (m, 1H), 2.24 (q, J=14.1, 13.6 Hz, 1H), 1.76-1.67 (m, 1H)⁻

### Example 124: Synthesis of Compound 410

### Synthesis of Intermediate C115

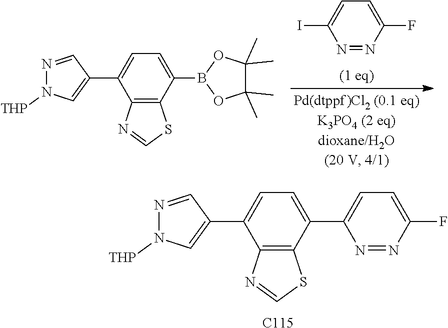

To a mixture of 4-(1-tetrahydropyran-2-ylpyrazol-4-yl)-7-(4,4,5,5-tetramethyl-1,3,2-dioxaborolan-2-yl)-1,3-benzothiazole (A5B2-10, 221 mg, 536 μmol, 1.2 eq) and 3-fluoro-6-iodo-pyridazine (1.00 g, 446 μmol, 1 eq) in dioxane (2 mL) and H₂O (0.5 mL) was added ditert-butyl(cyclopentyl)phosphane; dichloropalladium; iron (29.1 mg, 44.7 μmol, 0.1 eq) and K₃PO₄ (190 mg, 893 μmol, 2 eq) under N₂. The mixture was stirred at 80° C. for 1 hr. TLC (petroleum ether/ethyl acetate=1/1, Rf=0.15) showed a new spot was generated. The mixture was filtered, and the filtrate was concentrated to give a residue, which was purified by silica gel column chromatography (petroleum ether/ethyl acetate=1:1) to give 7-(6-fluoropyridazin-3-yl)-4-(1-tetrahydropyran-2-ylpyrazol-4-yl)-1,3-benzothiazole (C115; 0.120 g, 71%) as a solid. LCMS: (ES, m/z): 382.1 [M+H]⁺ ¹H NMR (400 MHz, DMSO-d₆) δ 9.60 (s, 1H), 8.9-8.9 (m, 2H), 8.46 (s, 1H), 8.36 (d, 1H, J=8.1 Hz), 8.11 (d, 1H, J=8.1 Hz), 7.92 (dd, 1H, J=1.2, 9.4 Hz), 5.54 (dd, 1H, J=2.3, 9.8 Hz), 3.99 (br d, 1H, J=10.9 Hz), 3.7-3.8 (m, 1H), 2.1-2.2 (m, 1H), 1.9-2.1 (m, 2H), 1.7-1.8 (m, 1H), 1.5-1.6 (m, 2H)

### Synthesis of Intermediate C116

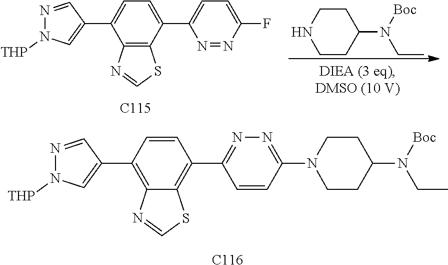

To a mixture of 7-(6-fluoropyridazin-3-yl)-4-(1-tetrahydropyran-2-ylpyrazol-4-yl)-1,3-benzothiazole (A5B2D1, 50.0 mg, 131 μmol, 1 eq) and tert-butyl N-ethyl-N-(4-piperidyl)carbamate (C7, 29.9 mg, 131 μmol, 1 eq) in dimethylsulfoxide (0.5 mL) was added diisopropylethylamine (50.8 mg, 393 μmol, 68.5 uL, 3 eq). The mixture was stirred at 130° C. for 14 hr. The reaction mixture was poured into H₂O (5 mL) and extracted with CH₂Cl₂ (5×10 mL). The organic layers were combined and washed with brine (10 mL), dried over anhydrous sodium sulfate, filtered and concentrated to give a residue, which was purified by silica gel column chromatography (petroleum ether/ethyl acetate=1:1) to give tert-butyl N-ethyl-N-[1-[6-[4-(1-tetrahydropyran-2-ylpyrazol-4-yl)-1,3-benzothiazol-7-yl]pyridazin-3-yl]-4-piperidyl]carbamate (C116; 30.0 mg, 39%) as a solid. LCMS: (ES, m/z): 590.2 [M+H]⁺ ¹H NMR (400 MHz, DMSO-d₆) δ 9.54 (s, 1H), 8.83 (s, 1H), 8.40 (s, 1H), 8.30 (d, 1H, J=9.9 Hz), 8.15 (d, 1H, J=8.3 Hz), 8.01 (d, 1H, J=7.8 Hz), 7.50 (d, 1H, J=9.8 Hz), 5.53 (dd, 1H, J=2.1, 9.8 Hz), 4.6-4.7 (m, 2H), 3.99 (br d, 1H, J=11.7 Hz), 3.6-3.8 (m, 2H), 3.1-3.1 (m, 2H), 3.0-3.0 (m, 2H), 2.1-2.2 (m, 1H), 2.01 (br d, 1H, J=2.8 Hz), 1.7-1.9 (m, 5H), 1.5-1.6 (m, 3H), 1.04 (t, 3H, J=6.9 Hz)

### Synthesis of Compound 410

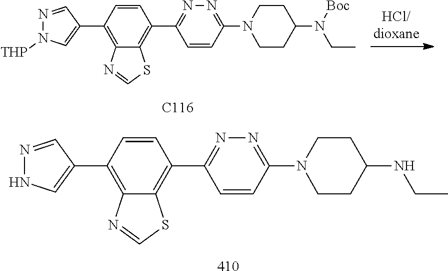

A solution of tert-butyl N-ethyl-N-[1-[6-[4-(1-tetrahydropyran-2-ylpyrazol-4-yl)-1,3-benzothiazol-7-yl]pyridazin-3-yl]-4-piperidyl]carbamate (30.0 mg, 50.9 μmol, 1 eq) in HCl (g)/dioxane (4 M, 5 mL) was stirred at 20° C. for 1 hr. The mixture was concentrated to give a residue. The crude product was purified by Prep-HPLC (Condition 14, Gradient 1) to give N-ethyl-1-[6-[4-(1H-pyrazol-4-yl)-1,3-benzothiazol-7-yl]pyridazin-3-yl]piperidin-4-amine (16.1 mg, 71.7%, HCl) as yellow solid. LCMS: (ES, m/z): 406.1 [M+H]⁺ ¹H NMR (400 MHz, METHANOL-d₄) δ 9.44 (s, 1H), 8.80 (s, 2H), 8.74 (d, 1H, J=10.3 Hz), 8.27 (d, 1H, J=8.1 Hz), 8.19 (d, 1H, J=10.1 Hz), 8.10 (d, 1H, J=8.0 Hz), 4.53 (br d, 2H, J=14.1 Hz), 3.62 (ddd, 1H, J=4.2, 7.2, 11.5 Hz), 3.5-3.6 (m, 2H), 3.20 (q, 2H, J=7.2 Hz), 2.41 (br d, 2H, J=10.7 Hz), 1.91 (dq, 2H, J=4.1, 12.1 Hz), 1.38 (t, 3H, J=7.3 Hz).

### Example 125: Synthesis of Compound 411

### Synthesis of Intermediate Cl₁₇

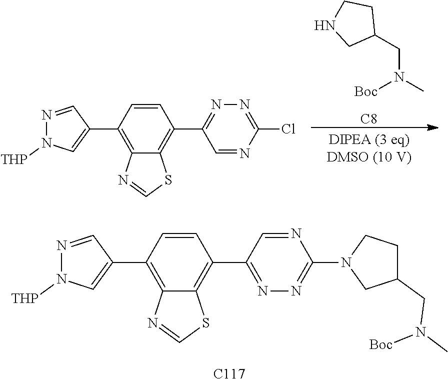

To a mixture of 7-(3-chloro-1,2,4-triazin-6-yl)-4-(1-tetrahydropyran-2-ylpyrazol-4-yl)-1,3-benzothiazole (50.0 mg, 125 μmol, 1 eq) and tert-butyl N-methyl-N-(pyrrolidin-3-ylmethyl)carbamate (C8, 26.9 mg, 125 μmol, 1 eq) in dimethylsulfoxide (0.5 mL) was added diisopropylethylamine (48.6 mg, 376 μmol, 65.5 uL, 3 eq). The mixture was stirred at 130° C. for 14 hr. The reaction mixture was poured into H₂O (10 mL) and extracted with CH₂Cl₂ (5×3 mL). The organic layers were combined and washed with saturated brine (10 mL), dried over anhydrous sodium sulfate, filtered and concentrated to give crude tert-butyl N-methyl-N-[[1-[6-[4-(1-tetrahydropyran-2-ylpyrazol-4-yl)-1,3-benzothiazol-7-yl]-1,2,4-triazin-3-yl]pyrrolidin-3-yl] methyl] carbamate (40.0 mg, 55%) as a solid. LCMS: (ES, m/z): 577.3 [M+H]⁺

### Synthesis of Compound 411

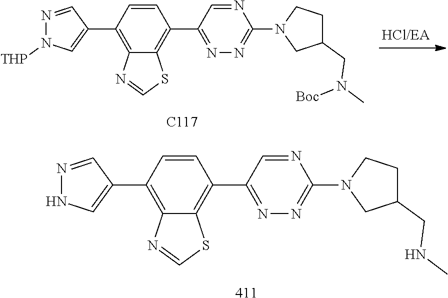

A solution of tert-butyl N-methyl-N-[[1-[6-[4-(1-tetrahydropyran-2-ylpyrazol-4-yl)-1,3-benzothiazol-7-yl]-1,2,4-triazin-3-yl]pyrrolidin-3-yl]methyl]carbamate (40.0 mg, 69.4 μmol, 1 eq) in HCl/ethyl acetate (4 M, 5.00 mL) was stirred at 20° C. for 1 hr. The mixture was concentrated to give a residue. The crude product was purified by prep-HPLC (Condition 14, Gradient 2) to give N-methyl-1-[1-[6-[4-(1H-pyrazol-4-yl)-1,3-benzothiazol-7-yl]-1,2,4-triazin-3-yl]pyrroledin-3-yl]metha-namine (Compound 411, 11.3 mg, 38% yield) as yellow solid. LCMS: (ES, m/z): 393.1 [M+H]⁺ ¹H NMR (400 MHz, METHANOL-d₄) δ 9.41 (s, 1H), 8.89 (br s, 2H), 8.0-8.1 (m, 1H), 7.97 (d, 1H, J=8.1 Hz), 6.05 (d, 1H, J=6.2 Hz), 3.9-4.0 (m, 1H), 3.8-3.9 (m, 1H), 3.7-3.8 (m, 1H), 3.4-3.5 (m, 1H), 3.2-3.3 (m, 2H), 2.8-3.0 (m, 1H), 2.80 (d, 3H, J=1.5 Hz), 2.4-2.5 (m, 1H), 1.9-2.1 (m, 1H)

### Example 126: Synthesis of Compound 412

### Synthesis of Intermediate C118

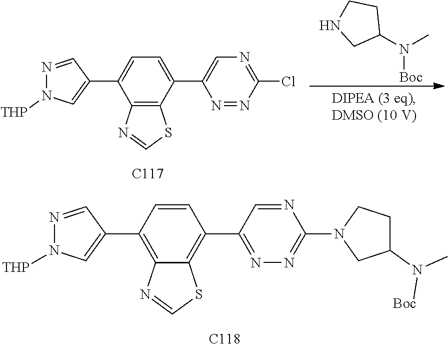

To a mixture of 7-(3-chloro-1,2,4-triazin-6-yl)-4-(1-tetrahydropyran-2-ylpyrazol-4-yl)-1,3-benzothiazole (50.0 mg, 125 μmol, 1 eq) and tert-butyl N-methyl-N-pyrrolidin-3-yl-carbamate (C9, 25.11 mg, 125 μmol, 1 eq) in dimethylsulfoxide (0.5 mL) was added diisopropylethylamine (48.6 mg, 376 μmol, 65.5 uL, 3 eq). The mixture was stirred at 130° C. for 14 hr. The reaction mixture was poured into H₂O (10 mL) and extracted with CH₂Cl₂ (5×20 mL). The organic layers were combined and washed with brine (10 mL), dried over anhydrous sodium sulfate, filtered and concentrated to give tert-butyl N-methyl-N-[1-[6-[4-(1-tetrahydropyran-2-ylpyrazol-4-yl)-1,3-benzothiazol-7-yl]-1,2,4-triazin-3-yl]pyrrolidin-3-yl]carbamate (40.0 mg, 57%) as a solid. LCMS: (ES, m/z): 563.3 [M+H]⁺

### Synthesis of Compound 412

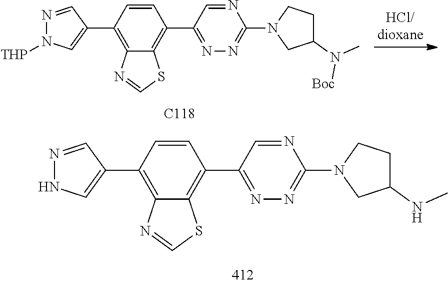

A solution of tert-butyl N-methyl-N-[1-[6-[4-(1-tetrahydropyran-2-ylpyrazol-4-yl)-1,3-benzothiazol-7-yl]-1,2,4-triazin-3-yl]pyrrolidin-3-yl]carbamate (40.0 mg, 71.1 μmol, 1 eq) in HCl/dioxane (4 M, 5.00 mL) was stirred at 20° C. for 1 hr. The mixture was concentrated to give a residue. The crude product was purified by prep-HPLC (Condition 14, Gradient 1) to give N-methyl-1-[6-[4-(1H-pyrazol-4-yl)-1,3-benzothiazol-7-yl]-1,2,4-triazin-3-yl]pyrrolidin-3-amine (Compound 412, 24.6 mg, 95%) as a solid. LCMS: (ES, m/z): 379.2 [M+H]⁺ ¹H NMR (400 MHz, METHANOL-d₄) δ 9.40 (s, 1H), 8.7-9.1 (m, 2H), 8.0-8.1 (m, 1H), 7.9-8.0 (m, 1H), 6.08 (s, 1H), 4.1-4.2 (m, 2H), 3.9-4.0 (m, 2H), 3.8-3.9 (m, 1H), 2.85 (d, 3H, J=1.2 Hz), 2.6-2.7 (m, 1H), 2.3-2.5 (m, 1H)

### Example 127: Synthesis of Compound 416

### Synthesis of Intermediate C119

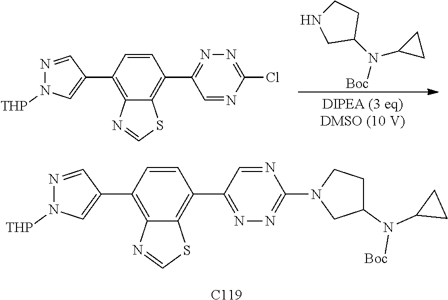

To a mixture of 7-(3-chloro-1,2,4-triazin-6-yl)-4-(1-tetrahydropyran-2-ylpyrazol-4-yl)-1,3-benzothiazole (30.0 mg, 75.2 μmol, 1 eq) and tert-butyl N-cyclopropyl-N-pyrrolidin-3-yl-carbamate (C6, 17.0 mg, 75.2 μmol, 1 eq) in dimethylsulfoxide (0.3 mL) was added diisopropylethylamine (29.2 mg, 226 μmol, 39.3 uL, 3 eq). The mixture was stirred at 130° C. for 14 hrs. TLC (petroleum ether/ethyl acetate=1/2, Rf=0.45) showed the starting material was consumed and a new major spot was generated. The reaction mixture was poured into H₂O (5 mL) and extracted with CH₂Cl₂ (4×3 mL). The organic layers were combined and washed with saturated brine (10 mL), dried over anhydrous sodium sulfate, filtered and concentrated to give a residue, which was purified by silica gel column chromatography (Petroleum ether/Ethyl acetate=1:1) to give tert-butyl N-cyclopropyl-N-[1-[6-[4-(1-tetrahydropyran-2-ylpyrazol-4-yl)-1,3-benzothiazol-7-yl]-1,2,4-triazin-3-yl]pyrrolidin-3-yl]carbamate (C119, 200 mg, 45%) as a solid. ¹H NMR (400 MHz, DMSO-d₆) δ 9.57 (s, 1H), 9.30 (s, 1H), 8.84 (s, 1H), 8.42 (s, 1H), 8.22 (d, 1H, J=8.2 Hz), 8.04 (d, 1H, J=7.9 Hz), 5.53 (dd, 1H, J=2.3, 9.7 Hz), 4.3-4.5 (m, 1H), 4.0-4.0 (m, 2H), 3.8-3.9 (m, 1H), 3.70 (ddd, 2H, J=4.3, 7.1, 14.0 Hz), 3.5-3.6 (m, 1H), 2.2-2.3 (m, 1H), 2.1-2.2 (m, 1H), 2.0-2.0 (m, 2H), 1.7-1.8 (m, 1H), 1.6-1.6 (m, 2H), 1.42 (s, 9H), 0.76 (br d, 2H, J=4.8 Hz), 0.66 (br d, 2H, J=3.1 Hz)

### Synthesis of Compound 416

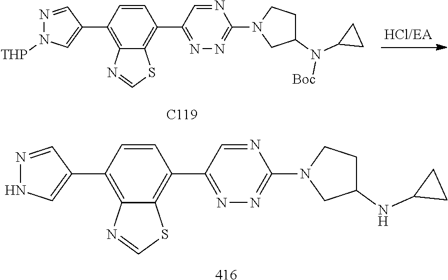

A solution of tert-butyl N-cyclopropyl-N-[1-[6-[4-(1-tetrahydropyran-2-ylpyrazol-4-yl)-1,3-benzothiazol-7-yl]-1,2,4-triazin-3-yl]pyrrolidin-3-yl]carbamate (200 mg, 34.0 μmol, 1 eq) in HCl/ethyl acetate (4 M, 3.00 mL) was stirred at 20° C. for 1 hr. The mixture was concentrated to give a residue. The crude product was purified by prep-HPLC (Condition 14, Gradient 3) to give N-cyclopropyl-1-[6-[4-(1H-pyrazol-4-yl)-1,3-benzothiazol-7-yl]-1,2,4-triazin-3-yl]py-rrolidin-3-amine (Compound 416; 4.19 mg, 40%) as a solid. LCMS: (ES, m/z): 405.1 [M+H]⁺1H NMR (400 MHz, METHANOL-d₄) δ 9.44 (s, 1H), 9.04 (s, 2H), 8.1-8.2 (m, 1H), 8.01 (d, 1H, J=8.1 Hz), 6.10 (d, 1H, J=1.9 Hz), 4.3-4.4 (m, 1H), 4.1-4.2 (m, 1H), 3.9-4.1 (m, 2H), 3.8-3.9 (m, 1H), 2.9-3.0 (m, 1H), 2.6-2.8 (m, 1H), 2.4-2.6 (m, 1H), 1.0-1.2 (m, 4H)

### Example 128: Synthesis of Compounds 423, 424, and 425

### Synthesis of Intermediate C120

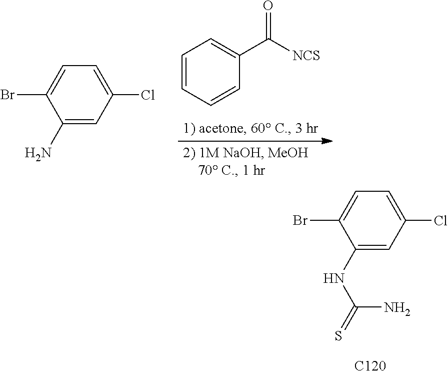

To a mixture of 2-bromo-5-chloro-aniline (50.0 g, 242 mmol, 1.0 eq) in acetone (500 mL) was added benzoyl isothiocyanate (43.5 g, 266 mmol, 35.9 mL, 1.1 eq) dropwise at 20° C. The mixture was stirred at 60° C. for 3 hrs. Then the reaction was poured into ice-water (1 L) and stirred for 0.5 hr. The precipitate was collected by filtration and washed with water (2×200 mL). The crude material was re-dissolved in CH₃OH (500 mL) and then treated with aq. NaOH solution (1 M, 250 mL, 1.03 eq). The reaction mixture was stirred at 70° C. for 1 hr. After cooling to 20° C., the reaction mixture was poured into ice-water (1.00 L) and the mixture was adjusted to pH=7 with aqueous HCl (6 N) at 0° C. The precipitate was collected by filtration and dried to give (2-bromo-5-chloro-phenyl) thiourea (C120; 55.0 g, 86%) as a solid. LCMS: (ES, m/z): 264.9 [M+H]⁺, ¹H NMR (400 MHz, DMSO-d₆) δ 9.31 (s, 1H), 7.9-8.2 (m, 1H), 7.76 (d, 1H, J=2.4 Hz), 7.67 (d, 1H, J=8.6 Hz), 7.3-7.6 (m, 1H), 7.23 (dd, 1H, J=2.5, 8.5 Hz).

### Synthesis of C121

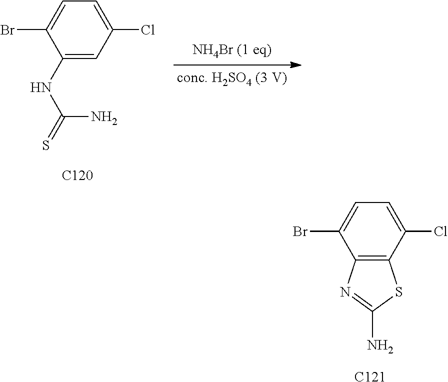 [SMILES: NC(=S)Nc1cc(Cl)ccc1Br ; "C120" ; Nc1nc2c(Br)ccc(Cl)c2s1 ; "C121"]

To a mixture of 2-bromo-5-chloro-phenyl)thiourea (9.00 g, 33.9 mmol, 1 eq) in H₂SO₄ (27 mL) was added ammonia; hydrobromide (3.32 g, 33.9 mmol, 1.37 mL, 1 eq) in portions. The mixture was stirred at 100° C. for 2 hrs. Four additional vials were set up as described above and five reaction mixtures were combined. After cooling to 20° C., the reaction mixture was poured into ice water slowly (200 mL) below 20° C. and the white precipitate was observed. The mixture was adjusted to pH=7 with aqueous ammonium hydroxide solution at 0° C. The solid was collected by filtration, washed with water (3×100 mL) and dried to give 4-bromo-7-chloro-1,3-benzothiazol-2-amine (C121, 40.0 g, 90%) as a solid. LCMS: (ES, m/z): 262.9 [M+H]⁺¹H NMR (400 MHz, DMSO-d₆) δ 8.13 (br s, 2H), 7.46 (br d, 1H, J=8.4 Hz), 7.03 (br d, 1H, J=8.3 Hz).

### Synthesis of Intermediate C122

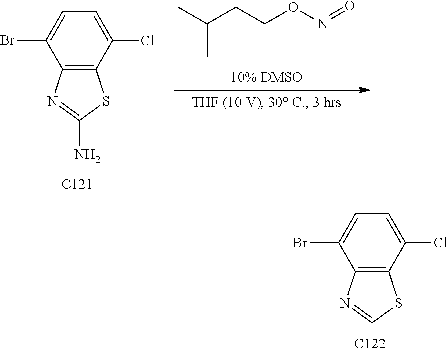

A solution of 4-bromo-7-chloro-1,3-benzothiazol-2-amine (10.0 g, 37.9 mmol, 1 eq) in tetrahydrofuran (70 mL) was added dropwise over 0.5 hr to a solution of isopentyl nitrite (6.67 g, 56.9 mmol, 7.66 mL, 1.5 eq) in a mixture of tetrahydrofuran (30 mL) and dimethysulfoxide (296 mg, 3.79 mmol, 296 μL, 0.1 eq) at 20° C. The mixture was stirred at 30° C. for 3 hrs. TLC (petroleum ether/ethyl acetate=1/1, Rf=0.9) showed the starting material was consumed and a new major spot was generated. Three additional vials were set up as described above and four reaction mixtures were combined. The mixture was poured into water (1.00 L) and extracted with ethyl acetate (3×300 mL). The organic layers were combined and washed with brine (1.00 L), dried over anhydrous sodium sulfate, filtered and concentrated to give a residue. The residue was triturated with petroleum ether (200 mL). The solid was collected by filtration and dried to give 4-bromo-7-chloro-1,3-benzothiazole (C122, 27.0 g, 72%) as a solid. ¹H NMR (400 MHz, DMSO-d₆) δ 9.58 (s, 1H), 7.86 (d, 1H, J=8.3 Hz), 7.58 (d, 1H, J=8.3 Hz)

### Synthesis of Intermediate C123

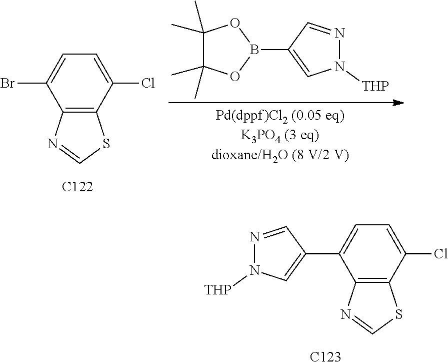

To a mixture of 4-bromo-7-chloro-1,3-benzothiazole (18.0 g, 72.4 mmol, 1 eq), 1-tetrahydropyran-2-yl-4-(4,4,5,5-tetramethyl-1,3,2-dioxaborolan-2-yl)pyrazole (22.2 g, 79.7 mmol, 1.1 eq) and K₃PO₄ (46.1 g, 217 mmol, 3 eq) in a mixture solvents of dioxane (144 mL) and H₂O (36 mL) was added 1,1′-bis(diphenylphosphino)ferrocenedichloro palladium(II) (2.65 g, 3.62 mmol, 0.05 eq) under N₂ protection. The mixture was stirred at 100° C. for 14 hrs. After cooling to 20° C., the reaction mixture was poured into H₂O (500 mL). The resulting mixture was extracted with ethyl acetate (4×200 mL). The combined organic phase was dried over anhydrous Na₂SO₄, filtered and concentrated to give a residue, which was purified by silica gel column chromatography (petroleum ether/ethyl acetate=10/1 to 1/1 over 60 min.) to give 7-chloro-4-(1-tetrahydropyran-2-ylpyrazol-4-yl)-1,3-benzothiazole (C123; 15.0 g, 65%) as a solid. LCMS: (ES, m/z): 320.0 [M+H]⁺ ¹H NMR (400 MHz, DMSO-d₆) δ 9.56 (s, 1H), 8.73 (s, 1H), 8.32 (s, 1H), 7.91 (d, 1H, J=8.2 Hz), 7.62 (d, 1H, J=8.1 Hz), 5.51 (dd, 1H, J=2.4, 9.8 Hz), 3.97 (br d, 1H, J=11.9 Hz), 3.6-3.7 (m, 1H), 2.0-2.2 (m, 1H), 1.9-2.0 (m, 2H), 1.6-1.8 (m, 1H), 1.5-1.6 (m, 2H)

### Synthesis of Intermediate C124

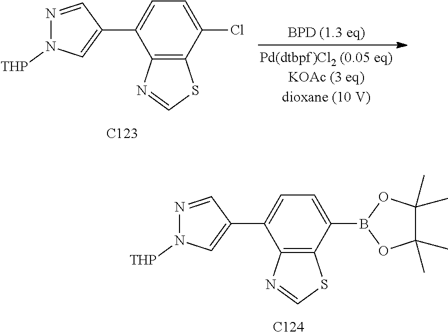

To a mixture of 7-chloro-4-(1-tetrahydropyran-2-ylpyrazol-4-yl)-1,3-benzothiazole (5.00 g, 15.6 mmol, 1 eq) in dioxane (50 mL) was added ditert-butyl(cyclopentyl)phosphane; dichloropalladium; iron (509 mg, 782 μmol, 0.05 eq), 4,4,4′,4′,5,5,5′,5′-octamethyl-2,2′-bi(1,3,2-dioxaborolane) (5.16 g, 20.3 mmol, 1.3 eq) and potassium acetate (4.60 g, 46.9 mmol, 3 eq) under N₂. The mixture was stirred at 80° C. for 8 hrs. After cooling to 20° C., the reaction mixture was filtered and the filter cake was washed with CH₂Cl₂ (3×30 mL). The filtrate was concentrated to give a residue, which was purified by silica gel column chromatography (petroleum ether/ethyl acetate=10/1 to 1/1 over 75 min.) to give 4-(1-tetrahydropyran-2-ylpyrazol-4-yl)-7-(4,4,5,5-tetramethyl-1,3,2-dioxaborolan-2-yl)-1,3-benzothiazole (C124, 2.30 g, 29%) as a solid. LCMS: (ES, m/z): 412.2 [M+H]⁺ ¹H NMR (400 MHz, DMSO-d₆) δ 9.47 (s, 1H), 8.82 (s, 1H), 8.36 (s, 1H), 7.92 (d, 1H, J=4.0 Hz), 7.79 (d, 1H, J=7.5 Hz), 5.51 (dd, 1H, J=2.4, 9.9 Hz), 3.97 (br d, 1H, J=11.9 Hz), 3.6-3.7 (m, 1H), 2.1-2.2 (m, 1H), 1.95 (br s, 2H), 1.7-1.8 (m, 1H), 1.5-1.6 (m, 2H), 1.36 (s, 12H).

### Synthesis of Intermediate C125

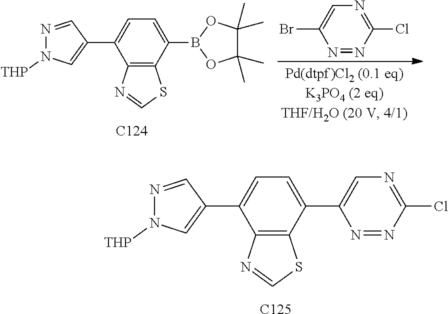

To a mixture of 6-bromo-3-chloro-1,2,4-triazine (800 mg, 4.11 mmol, 1 eq) and 4-(1-tetrahydropyran-2-ylpyrazol-4-yl)-7-(4,4,5,5-tetramethyl-1,3,2-dioxaborolan-2-yl)-1,3-benzothiazole (2.03 g, 4.94 mmol, 1.2 eq) in tetrahydrofuran (32 mL) and H₂O (8 mL) was added ditert-butyl(cyclopentyl)phosphane; dichloropalladium; iron (268 mg, 411 μmol, 0.1 eq) and K₃PO₄ (1.75 g, 8.23 mmol, 2 eq) under N₂. The mixture was stirred at 80° C. for 1 hr. After cooling to 20° C., the reaction mixture was filtered and the filter cake was washed with CH₂Cl₂ (3×30 mL). The filtrate was dried over anhydrous sodium sulfate, filtered and concentrated to give a residue, which was purified by silica gel column chromatography (petroleum ether/ethyl acetate=1:1, Rf=0.2) to give 7-(3-chloro-1,2,4-triazin-6-yl)-4-(1-tetrahydropyran-2-ylpyrazol-4-yl)-1,3-benzothiazole (C125; 550 mg, 34%) as a solid. LCMS: (ES, m/z): 399.1 [M+H]⁺¹H NMR (400 MHz, DMSO-d₆) δ 9.81 (s, 1H), 9.64 (s, 1H), 8.93 (s, 1H), 8.5-8.5 (m, 2H), 8.16 (d, 1H, J=8.1 Hz), 5.55 (dd, 1H, J=2.3, 9.8 Hz), 3.99 (br d, 1H, J=11.2 Hz), 3.6-3.8 (m, 1H), 2.1-2.2 (m, 1H), 1.9-2.1 (m, 2H), 1.7-1.8 (m, 1H), 1.5-1.6 (m, 2H)

### Synthesis of Intermediate C126

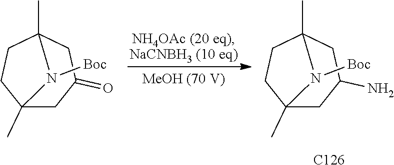 [SMILES: CC(C)(C)OC(=O)N1C2(C)CCC1(C)CC(=O)C2 ; "NH4OAc (20 eq),NaCNBH3 (10 eq)" ; CC(C)(C)OC(=O)N1C2(C)CCC1(C)CC(N)C2 ; "C126"]

To a mixture of tert-butyl (1R,5S)-1,5-dimethyl-3-oxo-8-azabicyclo[3.2.1]octane-8-carboxylate (300 mg, 1.18 mmol, 1 eq) in CH₃OH (21.0 mL) was added NH₄OAc (1.83 g, 23.7 mmol, 20 eq) and NaBH₃CN (744 mg, 11.8 mmol, 10 eq) under N₂. The mixture was stirred at 70° C. for 2 hr. TLC (petroleum ether/ethyl acetate=1/1, Rf=0) showed starting material was consumed and a new spot was generated. The reaction mixture was poured into saturated NaHCO₃ (60 mL) and extracted with ethyl acetate (5×20 mL). The organic layers were combined and dried over anhydrous sodium sulfate, filtered and concentrated to give tert-butyl 3-amino-1,5-dimethyl-8-azabicyclo[3.2.1]octane-8-carboxylate (C126, 240 mg, 78%) as an oil. ¹H NMR (400 MHz, DMSO-d₆) δ 3.3-3.3 (m, 1H), 3.0-3.1 (m, 1H), 2.2-2.2 (m, 1H), 1.8-2.1 (m, 3H), 1.5-1.6 (m, 3H), 1.3-1.4 (m, 16H), 1.2-1.3 (m, 2H)

### Synthesis of Intermediate C127

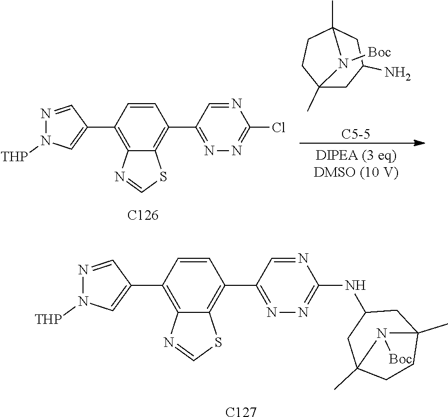

To a mixture of 7-(3-chloro-1,2,4-triazin-6-yl)-4-(1-tetrahydropyran-2-ylpyrazol-4-yl)-1,3-benzothiazole (100 mg, 251 μmol, 1 eq) and tert-butyl 3-amino-1,5-dimethyl-8-azabicyclo[3.2.1]octane-8-carboxylate (66.96 mg, 263.24 μmol, 1.05 eq) in dimethylsulfoxide (1 mL) was added diisopropylethylamine (97.21 mg, 752.13 μmol, 131.01 uL, 3 eq). The mixture was stirred at 130° C. for 14 hr. After cooling to 20° C., the reaction mixture was poured into H₂O (10 mL). The mixture was filtered and the solid was collected to give crude tert-butyl 1,5-dimethyl-3-[[6-[4-(1-tetrahydropyran-2-ylpyrazol-4-yl)-1,3-benzothiazol-7-yl]-1,2,4-triazin-3-yl]amino]-8 azabicyclo[3.2.1] octane-8-carboxylate (C127; 120 mg, 78%) as a solid. The product was used in the next step without further purification. LCMS: (ES, m/z): 617.3 [M+H]⁺.

### Synthesis of Intermediate C128

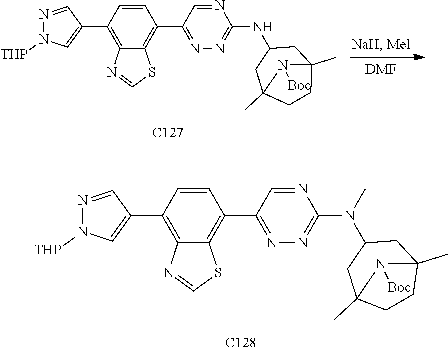

To a mixture of tert-butyl 1,5-dimethyl-3-[[6-[4-(1-tetrahydropyran-2-ylpyrazol-4-yl)-1,3-benzothiazol-7-yl]-1,2,4-triazin-3-yl]amino]-8-azabicyclo[3.2.1]octane-8-carboxylate (C127; 100 mg, 162 μmol, 1 eq) in dimethyl formamide (2 mL) was added NaH (9.73 mg, 243.20 μmol, 60% purity, 1.5 eq) in portions at 0° C. under N₂. The mixture was stirred at 0° C. for 30 min. Then, CH₃I (29.9 mg, 211 μmol, 13.1 uL, 1.3 eq) was added to the mixture at 0° C., and the mixture was stirred at 20° C. for an additional 1 hr. The reaction mixture was poured into H₂O (20 mL) and extracted with CH₂Cl₂ (5×10 mL). The combined organic layers were washed with saturated brine (30 mL), dried over anhydrous sodium sulfate, filtered and concentrated to give crude tert-butyl 1,5-dimethyl-3-[methyl-[6-[4-(1-tetrahydropyran-2-ylpyrazol-4-yl)-1,3-benzothiazol-7-yl]-1,2,4-triazin-3-yl]amino]-8-azabicyclo[3.2.1] octane-8-carboxylate (C128; 100 mg, 82%) as a solid. LCMS: (ES, m/z): 631.3 [M+H]⁺.

### Synthesis of Compound 423

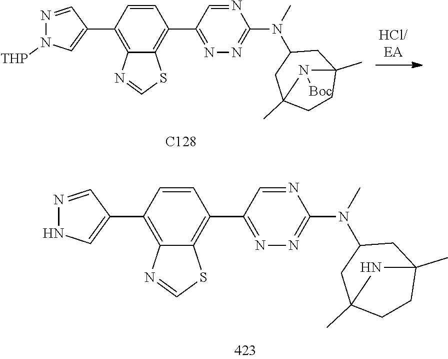

A solution of tert-butyl 1,5-dimethyl-3-[methyl-[6-[4-(1-tetrahydropyran-2-ylpyrazol-4-yl)-1,3-benzothiazol-7-yl]-1,2,4-triazin-3-yl]amino]-8-azabicyclo[3.2.1]octane-8-carboxylate (C128, 800 mg, 127 μmol, 1 eq) in HCl/ethyl acetate (4 M, 20 mL) was stirred at 20° C. for 1 hr. The mixture was concentrated to give a residue, which was purified by prep-HPLC (Condition 14, Gradient 4) to give N,1,5-trimethyl-N-[6-[4-(1H-pyrazol-4-yl)-1,3-benzothiazol-7-yl]-1,2,4-triazin-3-yl]-8-aza-bicyclo[3.2.1]octan-3-amine (Compound 423, 17.0 mg, 23%) as a solid. LCMS: (ES, m/z): 447.1 [M+H]⁺ ¹H NMR (400 MHz, METHANOL-d₄) δ 9.43 (s, 1H), 8.99 (br s, 2H), 8.08 (br d, 1H, J=7.9 Hz), 7.99 (br d, 1H, J=8.2 Hz), 6.0-6.1 (m, 1H), 4.50 (td, 1H, J=5.6, 11.2 Hz), 3.24 (s, 3H), 2.69 (br d, 1H, J=12.5 Hz), 2.3-2.4 (m, 3H), 2.0-2.2 (m, 4H), 1.6-1.7 (m, 6H).

### Separation of Compounds 424 and 425

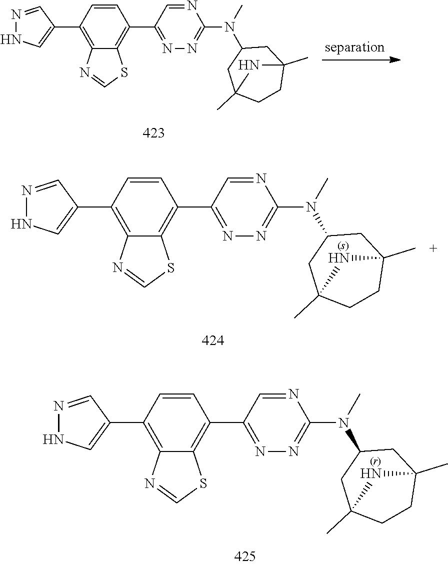 [SMILES: CN(c1ncc(-c2ccc(-c3cn[nH]c3)c3ncsc23)nn1)C1CC2(C)CCC(C)(C1)N2 ; CN(c1ncc(-c2ccc(-c3cn[nH]c3)c3ncsc23)nn1)[C@H]1C[C@]2(C)CC[C@](C)(C1)N2 ; CN(c1ncc(-c2ccc(-c3cn[nH]c3)c3ncsc23)nn1)[C@@H]1C[C@]2(C)CC[C@](C)(C1)N2]

N,1,5-trimethyl-N-[6-[4-(1H-pyrazol-4-yl)-1,3-benzothiazol-7-yl]-1,2,4-triazin-3-yl]-8-azabicyclo[3.2.1]octan-3-amine (Compound 423, 17.0 mg, 30.6 μmol) was separated by SFC (column: DAICEL CHIRALCEL OJ (250 mm*30 mm,10 μm); mobile phase: [0.1% NH₃H₂O isopropyl amine]; B %: 50%-50%, 9 min) to give (1S,5R)—N,1,5-trimethyl-N-[6-[4-(1H-pyrazol-4-yl)-1,3-benzothiazol-7-yl]-1,2,4-triazi n-3-yl]-8-azabicyclo [3.2.1] octan-3-amine (Compound 424, 3.90 mg, 37%) and (1S,5R)—N,1,5-trimethyl-N-[6-[4-(1H-pyrazol-4-yl)-1,3-benzothiazol-7-yl]-1,2,4-triazi n-3-yl]-8-azabicyclo [3.2.1] octan-3-amine (Compound 425, 2.00 mg, 15%) as a solid. Compound 424: LCMS: (ES, m/z): 447.1 [M+H]⁺ ¹H NMR (400 MHz, DMSO-d₆) δ 13.13 (br s, 1H), 9.54 (s, 1H), 9.28 (s, 1H), 8.55 (br s, 2H), 8.18 (d, 1H, J=8.1 Hz), 8.01 (d, 1H, J=7.9 Hz), 5.0-5.3 (m, 1H), 3.35 (br s, 1H), 3.11 (br s, 3H), 1.84 (br d, 2H, J=6.7 Hz), 1.7-1.8 (m, 2H), 1.61 (br d, 4H, J=6.5 Hz), 1.24 (s, 6H) Compound 425: LCMS: (ES, m/z): 447.1 [M+H]⁺ ¹H NMR (400 MHz, DMSO-d₆) δ 12.9-13.3 (m, 1H), 9.54 (s, 1H), 9.32 (s, 1H), 9.07 (br s, 1H), 8.4-8.7 (m, 2H), 8.24 (d, 1H, J=7.9 Hz), 8.02 (d, 1H, J=7.9 Hz), 5.47 (br t, 1H, J=9.4 Hz), 3.17 (s, 3H), 2.1-2.2 (m, 6H), 1.96 (br d, 2H, J=7.7 Hz), 1.51 (s, 6H).

### Example 129: Synthesis of Compound 444

### Synthesis of Intermediate C129

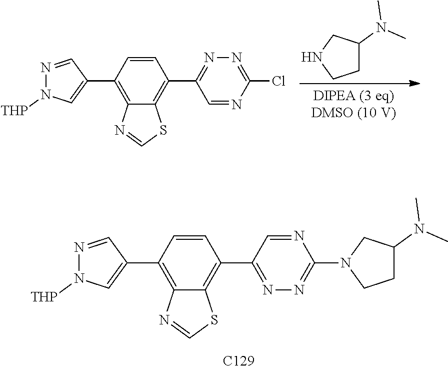

To a mixture of 7-(3-chloro-1,2,4-triazin-6-yl)-4-(1-tetrahydropyran-2-ylpyrazol-4-yl)-1,3-benzothiazole (50.0 mg, 125 μmol, 1 eq) and N,N-dimethylpyrrolidin-3-amine (C11, 14.3 mg, 125 μmol, 1 eq) in dimethylsulfoxide (0.5 mL) was added diisopropylethylamine (48.6 mg, 376 μmol, 65.5 uL, 3 eq). The mixture was stirred at 130° C. for 14 hr. The reaction mixture was poured into H₂O (10 mL), and extracted with CH₂Cl₂ (5×20 mL). The organic layers were combined and washed with brine (10 mL), dried over anhydrous sodium sulfate, filtered and concentrated to give crude N,N-dimethyl-1-[6-[4-(1-tetrahydropyran-2-ylpyrazol-4-yl)-1,3-benzothiazol-7-yl]-1,2,4-triazin-3-yl] pyrrolidin-3-amine (C129, 40.0 mg, 67%) as a solid. LCMS: (ES, m/z): 477.2 [M+H]⁺.

### Synthesis of Compound 444

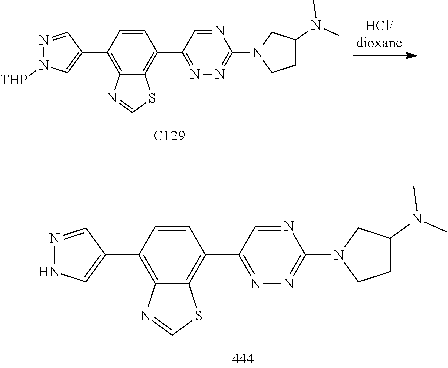

A solution of N,N-dimethyl-1-[6-[4-(1-tetrahydropyran-2-ylpyrazol-4-yl)-1,3-benzothiazol-7-yl]-1,2,4-triazin-3-yl]pyrrolidin-3-amine (C129, 40.0 mg, 83.9 μmol, 1 eq) in HCl (g)/dioxane (4 M, 5.00 mL) was stirred at 20° C. for 1 hr. The mixture was concentrated to give a residue. The crude product was purified by reversed-phase HPLC (Condition 14, Gradient 5) to give N,N-dimethyl-1-[6-[4-(1H-pyrazol-4-yl)-1,3-benzothiazol-7-yl]-1,2,4-triazin-3-yl]pyrrolidin-3-amine (EVAL-0118-0034, 18.9 mg, 65% yield) as solid. LCMS: (ES, m/z): 393.1 [M+H]⁺ ¹H NMR (400 MHz, METHANOL-d₄) δ 9.40 (s, 1H), 8.82 (s, 2H), 8.0-8.1 (m, 1H), 7.9-8.0 (m, 1H), 6.0-6.1 (m, 1H), 4.1-4.3 (m, 2H), 3.9-4.1 (m, 2H), 3.7-3.8 (m, 1H), 3.04 (d, 6H, J=3.9 Hz), 2.6-2.8 (m, 1H), 2.4-2.6 (m, 1H).

### Example 130: Synthesis of Compound 449

### Synthesis of Intermediate C130

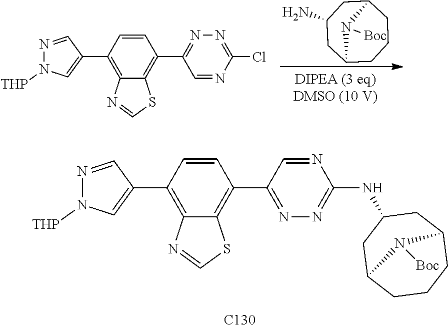

To a mixture of 7-(3-chloro-1,2,4-triazin-6-yl)-4-(1-tetrahydropyran-2-ylpyrazol-4-yl)-1,3-benzothiazole (C130, 50.0 mg, 125 μmol, 1 eq) and tert-butyl (1S,5R)-3-amino-9-azabicyclo[3.3.1]nonane-9-carboxylate (30.13 mg, 125 μmol, 1 eq) in dimethylsulfoxide (0.5 mL) was added diisopropylethylamine (C130, 48.6 mg, 376 μmol, 65.5 uL, 3 eq). The mixture was stirred at 130° C. for 14 hr. The reaction mixture was poured into H₂O (10 mL). The precipitate was collected by filtration and dried to give crude tert-butyl (1S,5R)-3-[[6-[4-(1-tetrahydropyran-2-ylpyrazol-4-yl)-1,3-benzothiazol-7-yl]-1,2,4-triazin-3-yl]amino]-9-azabicyclo[3.3.1]-nonane-9-carboxylate (C130, 50.0 mg, 66%) as a solid. The product was used in the next step directly without purification. LCMS: (ES, m/z): 603.4 [M+H]⁺.

### Synthesis of Intermediate C131

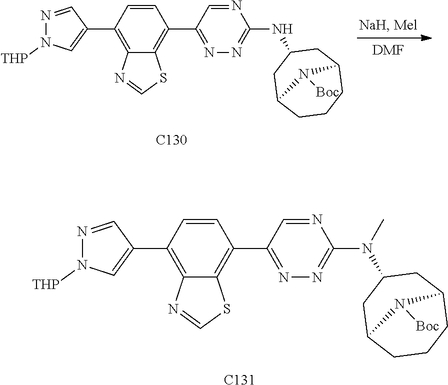

To a mixture of tert-butyl (1S,5R)-3-[[6-[4-(1-tetrahydropyran-2-ylpyrazol-4-yl)-1,3-benzothiazol-7-yl]-1,2,4-triazin-3-yl]amino]-9-azabicyclo[3.3.1]nonane-9-carboxylate (C130, 50.0 mg, 83.0 μmol, 1 eq) in dimethyl formamide (1 mL) was added NaH (4.98 mg, 124 μmol, 60% purity, 1.5 eq) in portions at 0° C. under N₂. The mixture was stirred at 0° C. for 30 min. Then CH₃I (17.7 mg, 124 μmol, 7.75 uL, 1.5 eq) was added to the mixture at 0° C., and the mixture was stirred at 20° C. for 2 hrs. LCMS showed 65% of product with desired MS was detected. The reaction mixture was poured into H₂O (10 mL), and extracted with CH₂Cl₂ (5×3 mL). The organic layers were combined and washed with brine (10 mL), dried over anhydrous sodium sulfate, filtered and concentrated to give tert-butyl (1S,5R)-3-[methyl-[6-[4-(1-tetrahydropyran-2-ylpyrazol-4-yl)-1,3-benzothiazol-7-yl]-1,2,4-triazin-3-yl]amino]-9-azabicyclo[3.3.1]nonane-9-carboxylate (C131, 50.0 mg, 98%) as a solid. LCMS: (ES, m/z): 617.1 [M+H]⁺.

### Synthesis of Compound 449

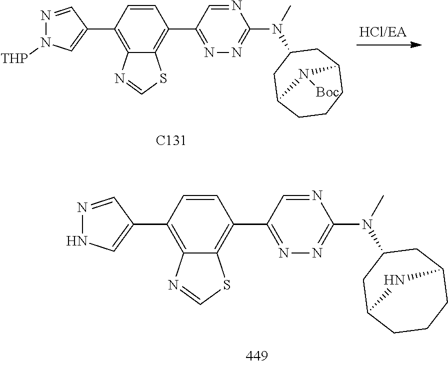

A solution of tert-butyl (1S,5R)-3-[methyl-[6-[4-(1-tetrahydropyran-2-ylpyrazol-4-yl)-1,3-benzothiazol-7-yl]-1,2,4-triazin-3-yl]amino]-9-azabicyclo[3.3.1]nonane-9-carbox-ylate (C131, 50.0 mg, 81.1 μmol, 1 eq) in HCl (g)/ethyl acetate (4 M, 20.0 mL) was stirred at 20° C. for 1 hr. The mixture was concentrated to give a residue. The residue was purified by prep-HPLC (Condition 14, Gradient 6) to give (1S,5R)—N-methyl-N-[6-[4-(1H-pyrazol-4-yl)-1,3-benzothiazol-7-yl]-1,2,4-triazin-3-yl]-9-azabicyclo[3.3.1]nonan-3-amine (Compound 449; 6.22 g, 20%) as a solid. LCMS: (ES, m/z): 433.1 [M+H]⁺ 1H NMR (400 MHz, METHANOL-d₄) δ 9.42 (s, 1H), 8.90 (s, 2H), 8.08 (d, 1H, J=8.1 Hz), 8.00 (d, 1H, J=7.9 Hz), 6.08 (s, 1H), 4.9-5.0 (m, 1H), 3.89 (br s, 2H), 3.20 (s, 3H), 2.5-2.6 (m, 2H), 2.30 (br dd, 1H, J=6.4, 14.7 Hz), 2.0-2.2 (m, 6H), 1.8-1.9 (m, 1H).

### Example 130: Synthesis of Compound 452

### Synthesis of Intermediate C132

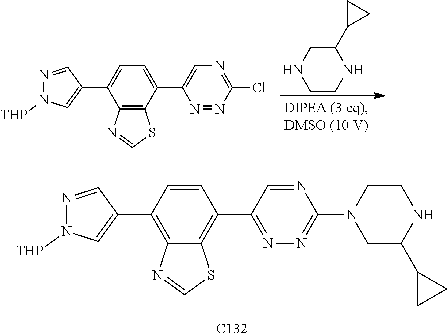

To a mixture of 7-(3-chloro-1,2,4-triazin-6-yl)-4-(1-tetrahydropyran-2-ylpyrazol-4-yl)-1,3-benzothiazole (50.0 mg, 125 μmol, 1 eq) and 2-cyclopropylpiperazine (C10, 15.8 mg, 125 μmol, 1 eq) in dimethylsulfoxide (0.5 mL) was added diisopropylethylamine (48.6 mg, 376 μmol, 65.5 uL, 3 eq). The mixture was stirred at 130° C. for 14 hrs. LCMS showed 69% of product with desired MS was detected. The reaction mixture was poured into H₂O (10 mL) and extracted with CH₂Cl₂ (5×3 mL). The organic layers were combined and washed with brine (10 mL), dried over anhydrous sodium sulfate, filtered and concentrated to give crude 7-[3-(3-cyclopropylpiperazin-1-yl)-1,2,4-triazin-6-yl]-4-(1-tetrahydropyran-2-ylpyrazol-4-yl)-1,3-benzothiazole (C132, 40.0 mg, 65%) as a solid. LCMS: (ES, m/z): 489.2 [M+H]⁺.

### Synthesis of Compound 452

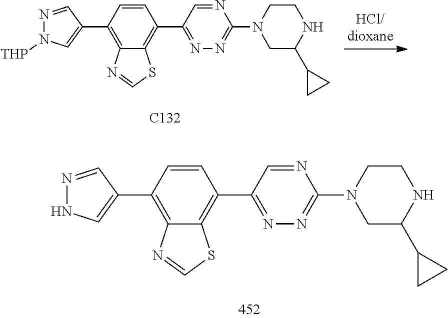

A solution of 7-[3-(3-cyclopropylpiperazin-1-yl)-1,2,4-triazin-6-yl]-4-(1-tetrahydropyran-2-ylpyrazol-4-yl)-1,3-benzothiazole (40.0 mg, 81.9 μmol, 1 eq) in HCl (g)/dioxane (4 M, 5.00 mL) was stirred at 20° C. for 1 hr. The mixture was concentrated to give a residue. The crude product was purified by prep HPLC (Condition 14, Gradient 7) to give 7-[3-(3-cyclopropylpiperazin-1-yl)-1,2,4-triazin-6-yl]-4-(1H-pyrazol-4-yl)-1,3-benzothiazole (Compound 452, 34.4 mg, 93%) as a solid. LCMS: (ES, m/z): 405.1 [M+H]⁺ 1H NMR (400 MHz, DEUTERIUM OXIDE) 6 9.01 (s, 1H), 8.10 (s, 2H), 8.03 (br s, 1H), 7.2-7.2 (m, 1H), 7.1-7.1 (m, 1H), 4.1-4.3 (m, 2H), 3.4-3.5 (m, 1H), 3.0-3.2 (m, 1H), 2.9-3.0 (m, 2H), 2.2-2.4 (m, 1H), 0.9-1.0 (m, 1H), 0.7-0.8 (m, 2H), 0.3-0.5 (m, 2H).

### Example 131: Synthesis of Compound 454

### Synthesis of Intermediate C133

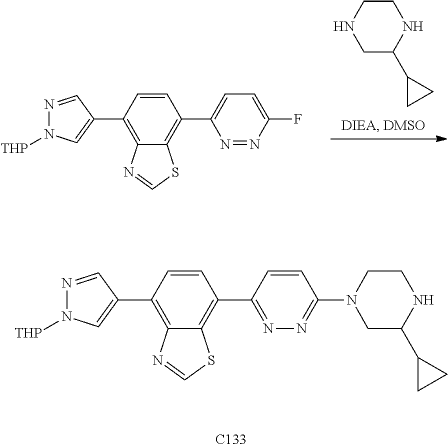

To a mixture of 7-(6-fluoropyridazin-3-yl)-4-(1-tetrahydropyran-2-ylpyrazol-4-yl)-1,3-benzothiazole (100 mg, 262 μmol, 1 eq) and 2-cyclopropylpiperazine (33.1 mg, 262 μmol, 1 eq) in dimethylsulfoxide (1 mL) was added diisopropylethylamine (102 mg, 787 μmol, 137 μL, 3 eq). The mixture was stirred at 130° C. for 14 hr. The reaction mixture was poured into H₂O (10 mL). The precipitate was collected by filtration and dried to give 7-[6-(3-cyclopropylpiperazin-1-yl)pyridazin-3-yl]-4-(1-tetrahydropyran-2-ylpyrazol-4-yl)-1,3-benzothiazole (C133, 100 mg, 78%) as a solid. LCMS: (ES, m/z): 488.3 [M+H]⁺.

### Synthesis of Compound 454

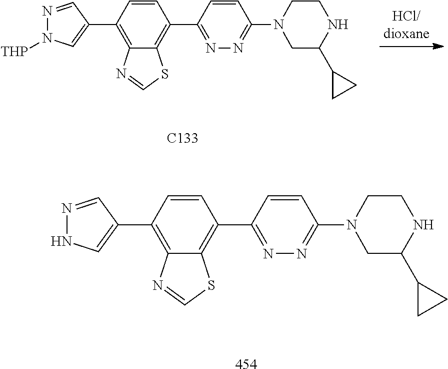

A mixture of 7-[6-(3-cyclopropylpiperazin-1-yl)pyridazin-3-yl]-4-(1-tetrahydropyran-2-ylpyrazol-4-yl)-1,3-benzothiazole (100 mg, 205 μmol, 1 eq) in HCl (g)/dioxane (4 M, 20.0 mL) was stirred at 20° C. for 1 hr. The reaction mixture was concentrated to give a solid. The solid was purified by prep-(Condition 14, Gradient 1) to give 7-[6-(3-cyclopropylpiperazin-1-yl)pyridazin-3-yl]-4-(1H-pyrazol-4-yl)-1,3-benzothiaz-ole (Compound 454, 61.1 mg, 93.0%) as a solid. LCMS: (ES, m/z): 404.1 [M+H]⁺ ¹H NMR (400 MHz, METHANOL-d₄) δ 9.44 (s, 1H), 8.84 (s, 2H), 8.71 (d, 1H, J=10.1 Hz), 8.25 (d, 1H, J=8.1 Hz), 8.1-8.1 (m, 2H), 4.67 (br d, 1H, J=14.2 Hz), 4.55 (br d, 1H, J=14.4 Hz), 3.7-3.8 (m, 3H), 3.40 (br dd, 1H, J=9.2, 12.0 Hz), 2.88 (dt, 1H, J=3.3, 10.4 Hz), 1.1-1.2 (m, 1H), 0.86 (quin, 2H, J=8.4 Hz), 0.67 (br d, 2H, J=4.6 Hz).

### Example 132: Synthesis of Compound 507

### Synthesis of Intermediate C134

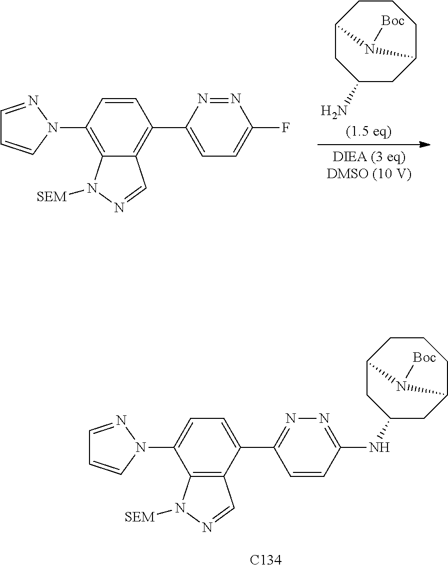 [SMILES: C[Si](C)(C)CCOCn1ncc2c(-c3ccc(F)nn3)ccc(-n3cccn3)c21 ; CC(C)(C)OC(=O)N1[C@@H]2CCC[C@H]1C[C@H](N)C2 ; CC(C)(C)OC(=O)N1[C@@H]2CCC[C@H]1C[C@H](Nc1ccc(-c3ccc(-n4cccn4)c4c3cnn4COCC[Si](C)(C)C)nn1)C2 ; "C134"]

A mixture of 2-[[4-(6-fluoropyridazin-3-yl)-7-pyrazol-1-yl-indazol-1-yl]methoxy]-ethyl-trimethyl-silane (70.0 mg, 170 μmol, 1 eq), diisopropylethylamine (66.1 mg, 511 μmol, 3 eq), tert-butyl (1S,5R)-3-amino-9-azabicyclo[3.3.1]nonane-9-carboxylate (61.5 mg, 255 μmol, 1.5 eq) in dimethyl sulfoxide (0.70 mL) was stirred at 130° C. for 16 hrs. LCMS showed the starting material was consumed and 95% of product with desired MS was detected. After cooled to 20° C., the reaction mixture was poured into H₂O (20 mL). The resulting mixture was extracted with dichloromethane (5×10 mL). The organic layer was dried over Na₂SO₄, filtered and the filtrate was concentrated under reduced pressure to give crude tert-butyl (1R,5S)-3-[[6-[7-pyrazol-1-yl-1-(2-trimethylsilylethoxymethyl)indazol-4-yl]pyridazin-3-yl] amino]-9-azabicyclo[3.3.1]-nonane-9-carboxylate (C134, 70.0 mg, 65%) as a solid. The crude product was used in the next step directly without further purification. LCMS: (ES, m/z): 631.2 [M+H]⁺.

### Synthesis of Intermediate C135

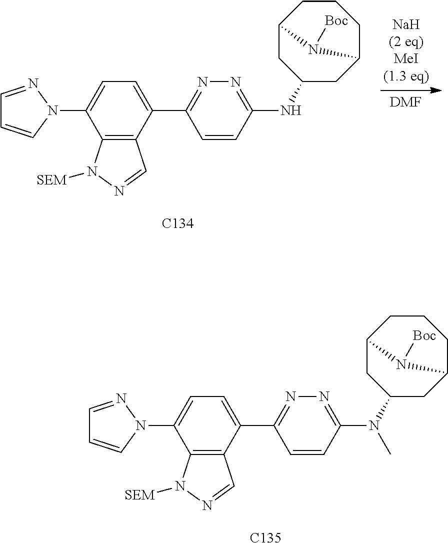

To a solution of tert-butyl (1R,5S)-3-[[6-[7-pyrazol-1-yl-1-(2-trimethylsilylethoxy methyl)indazol-4-yl]pyridazin-3-yl]amino]-9-azabicyclo[3.3.1]-nonane-9-carboxylate (70 mg, 111 μmol, 1 eq) in N,N-dimethylformamide (3.50 mL) was added sodium hydride (60 wt %, 8.88 mg, 222 μmol, 2 eq) at 0° C. in portions. After completion of addition, the mixture was stirred at 0° C. for 30 mins. Then iodomethane (20.5 mg, 144 μmol, 1.3 eq) was added dropwise at 0° C. The resulting mixture was stirred at 25° C. for 16 hrs. LCMS showed the starting material was consumed and 88% of product with desired MS was detected. The reaction mixture was quenched with H₂O (20 mL) at 5° C. and extracted with dichloromethane (5×10 mL). The organic layer was dried over Na₂SO₄, filtered and the filtrate was concentrated under reduced pressure to give crude tert-butyl (1R,5S)-3-[methyl-[6-[7-pyrazol-1-yl-1-(2-trimethylsilylethoxymethyl)indazol-4-yl]pyridazin-3-yl]amino]-9-azabicyclo[3.3.1] nonane-9-carboxylate (C135, 70.0 mg, 59%) as a solid. The crude product was used in the next step directly without further purification. LCMS: (ES, m/z): 645.3 [M+H]⁺.

### Synthesis of Compound 507

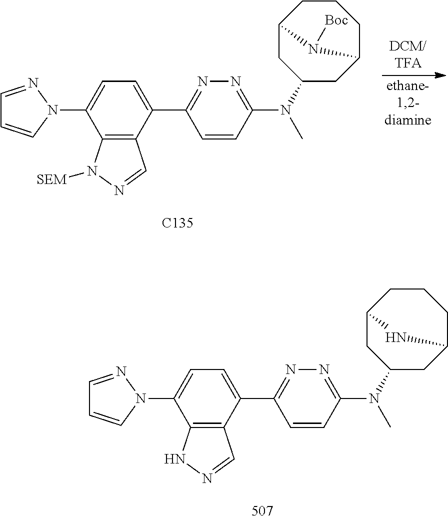 [SMILES: CN(c1ccc(-c2ccc(-n3cccn3)c3c2cnn3COCC[Si](C)(C)C)nn1)[C@@H]1C[C@H]2CCC[C@@H](C1)N2C(=O)OC(C)(C)C ; "ethane-1,2-diamine" ; "C135" ; CN(c1ccc(-c2ccc(-n3cccn3)c3[nH]ncc23)nn1)[C@@H]1C[C@H]2CCC[C@@H](C1)N2 ; "507"]

To a solution of tert-butyl (1R,5S)-3-[methyl-[6-[7-pyrazol-1-yl-1-(2-trimethylsilyl ethoxymethyl)indazol-4-yl]pyridazin-3-yl]amino]-9-azabicyclo[3.3.1]-nonane-9-carboxylate (100 mg, 155 μmol, 1 eq) in dichloromethane (5.0 mL) was added dropwise trifluoroacetic acid (1.51 mg, 1.32 mmol, 8.52 eq). After completion of addition, the mixture was stirred at 20° C. for 12 hrs. Then a solution of ethane-1,2-diamine (898 mg, 14.9 mmol, 96.3 eq) in dichloromethane (0.15 mL) was added dropwise to the reaction mixture at 20° C. The resulting mixture was stirred for additional 2 hrs. The reaction mixture was quenched with NaHCO₃ (10 mL) at 20° C., and extracted with dichloromethane (5×5 mL). The combined organic layers were dried over Na₂SO₄, filtered and concentrated under reduced pressure to give a residue. The residue was purified by prep-HPLC (Condition 14, Gradient 8) to give (1R,5S)-N-methyl-N-[6-(7-pyrazol-1-yl-1H-indazol-4-yl) pyridazin-3-yl]-9-azabicyclo[3.3.1]nonan-3-amine (Compound 507, 21.8 mg, 33%) as a solid. LCMS: (ES, m/z): 415.1 [M+H]⁺. ¹H NMR (400 MHz, DMSO-d₆) δ ppm 8.70-8.79 (m, 2H) 8.42 (d, J=10.03 Hz, 1H) 7.95 (s, 1H) 7.88-7.92 (m, 1H) 7.79 (br d, J=8.68 Hz, 1H) 6.69 (t, J=1.83 Hz, 1H) 5.29-5.45 (m, 1H) 3.78 (br s, 2H) 3.10 (s, 3H) 2.36 (td, J=13.17, 4.95 Hz, 2H) 2.07-2.15 (m, 1H) 1.90-2.05 (m, 6H) 1.67-1.80 (m, 1H)

### Example 133: Synthesis of Compound 512

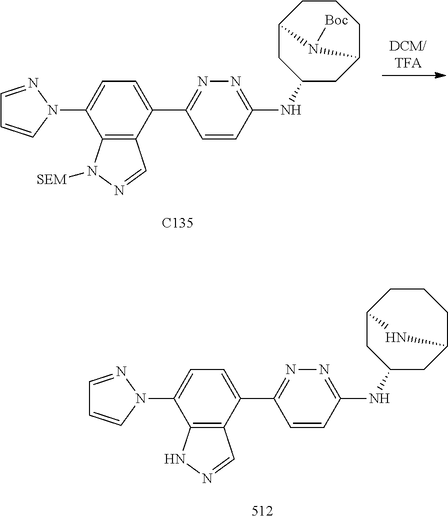 [SMILES: CC(C)(C)OC(=O)N1[C@@H]2CCC[C@H]1C[C@H](Nc1ccc(-c3ccc(-n4cccn4)c4c3cnn4COCC[Si](C)(C)C)nn1)C2 ; "C135" ; c1cnn(-c2ccc(-c3ccc(N[C@@H]4C[C@H]5CCC[C@@H](C4)N5)nn3)c3cn[nH]c23)c1 ; "512"]

To a solution of tert-butyl (1R,5S)-3-[[6-[7-pyrazol-1-yl-1-(2-trimethylsilylethoxy methyl)indazol-4-yl]pyridazin-3-yl]amino]-9-azabicyclo[3.3.1]-nonane-9-carboxylate (30.0 mg, 47.5 μmol, 1 eq) in dichloromethane (0.150 mL) was added dropwise trifluoroacetic acid (46.2 mg, 405 μmol, 8.52 eq). After addition, the mixture was stirred at 20° C. for 12 hrs. The reaction mixture was quenched with NaHCO₃ (10 mL) at 20° C. The reaction mixture was extracted with dichloromethane (5×10 mL). The combined organic layers were combined, dried over Na₂SO₄, filtered and concentrated under reduced pressure to give a residue. The residue was purified by prep-HPLC (Condition 14, Gradient 5) to give (1R,5S)-N-[6-(7-pyrazol-1-yl-1H-indazol-4-yl)pyridazin-3-yl]-9-azabicyclo[3.3.1]-nonan-3-amine (Compound 512, 5.11 mg, 27%) as a solid. LCMS: (ES, m/z): 401.1 [M+H]⁺. ¹H NMR (400 MHz, DMSO-d₆) δ ppm 8.74-8.87 (m, 1H) 8.66-8.73 (m, 1H) 8.45 (br d, J=9.90 Hz, 1H) 7.88-7.99 (m, 2H) 7.83 (d, J=7.95 Hz, 1H) 7.60 (br d, J=9.78 Hz, 1H) 6.69 (d, J=1.96 Hz, 1H) 4.66-4.78 (m, 1H) 3.74 (br s, 2H) 2.37 (br dd, J=13.57, 5.50 Hz, 2H) 2.07-2.20 (m, 1H) 1.90-2.06 (m, 6H) 1.63-1.72 (m, 1H).

### Example 134: Synthesis of Compound 541

### Synthesis of Intermediate C136

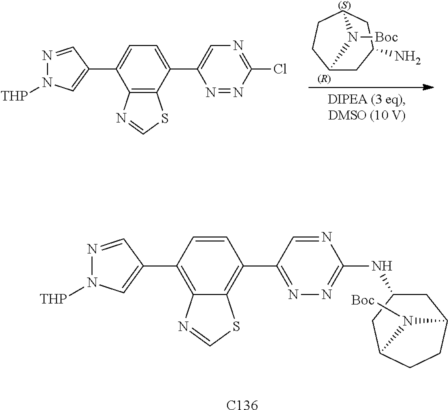

To a mixture of 7-(3-chloro-1,2,4-triazin-6-yl)-4-(1-tetrahydropyran-2-ylpyrazol-4-yl)-1,3-benzothiazole (40.0 mg, 100 μmol, 1 eq) and tert-butyl (1S,5R)-3-amino-8-azabicyclo[3.2.1]octane-8-carboxylate (22.7 mg, 100 μmol, 1 eq) in dimethylsulfoxide (0.4 mL) was added diisopropylethylamine (38.9 mg, 301 μmol, 52.4 uL, 3 eq). The mixture was stirred at 130° C. for 14 hrs. The reaction mixture was poured into H₂O (10 mL). The precipitate was collected by filtration and dried to give tert-butyl (1R,5S)-3-[[6-[4-(1-tetrahydropyran-2-ylpyrazol-4-yl)-1,3-benzothiazol-7-yl]-1,2,4-triazin-3-yl]amino]-8-azabicyclo[3.2.1]-octane-8-carboxylate (C136, 40.0 mg, 68%) as a solid. LCMS: (ES, m/z): 589.2 [M+H]⁺.

### Synthesis of Compound 541

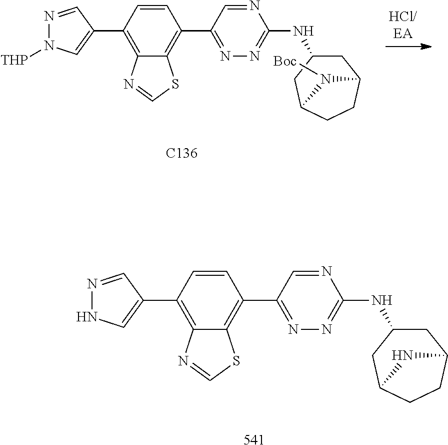

A solution of tert-butyl (1R,5S)-3-[[6-[4-(1-tetrahydropyran-2-ylpyrazol-4-yl)-1,3-benzothiazol-7-yl]-1,2,4-triazin-3-yl]amino]-8-azabicyclo[3.2.1]octane-8-carboxylate (C136, 20.0 mg, 34.0 μmol, 1 eq) in HCl (g)/ethyl acetate (4 M, 5.00 mL) was stirred at 20° C. for 1 hr. One additional vial was set up as described above and two reaction mixtures were combined. The mixture was concentrated to give a residue. The residue was purified by prep-HPLC (Condition 14, Gradient 7) to give (1R,5S)-N-[6-[4-(1H-pyrazol-4-yl)-1,3-benzothiazol-7-yl]-1,2,4-triazin-3-yl]-8-azabicyclo[3.2.1]octan-3-amine (Compound 541; 4.14 mg, 20%) as a solid. LCMS: (ES, m/z): 405.0 [M+H]⁺ 1H NMR (400 MHz, METHANOL-d₄) δ 9.41 (s, 1H), 9.00 (br s, 2H), 8.0-8.1 (m, 1H), 7.93 (br d, 1H, J=7.9 Hz), 6.07 (s, 1H), 4.31 (br s, 1H), 4.19 (br s, 2H), 2.3-2.5 (m, 4H), 2.0-2.2 (m, 4H).

### Example 135: Synthesis of Compound 525

### Synthesis of C137

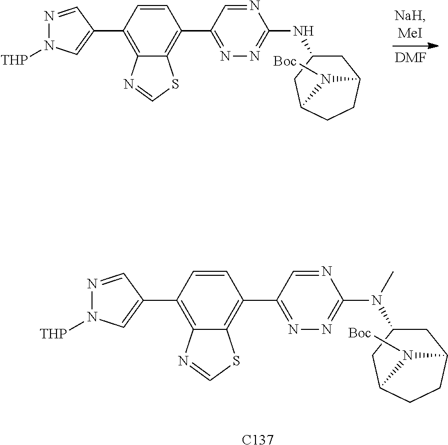

To a mixture of tert-butyl (1R,5S)-3-[[6-[4-(1-tetrahydropyran-2-ylpyrazol-4-yl)-1,3-benzothiazol-7-yl]-1,2,4-triazin-3-yl]amino]-8-azabicyclo[3.2.1]octane-8-carboxylate (50.0 mg, 84.9 μmol, 1 eq) in dimethyl formamide (1 mL) was added NaH (5.10 mg, 127 μmol, 60% purity, 1.5 eq) in portions at 0° C. under N₂. The mixture was stirred at 0° C. for 30 min. Then CH₃I (18.1 mg, 127 μmol, 7.93 uL, 1.5 eq) was added to the mixture at 0° C. The mixture was stirred at 20° C. for 2 hrs. The reaction mixture was poured into H₂O (10 mL), and extracted with CH₂Cl₂ (5×3 mL). The organic layers were combined and washed with brine (10 mL), dried over anhydrous sodium sulfate, filtered and concentrated to give tert-butyl (1R,5S)-3-[methyl-[6-[4-(1-tetrahydropyran-2-ylpyrazol-4-yl)-1,3-benzothiazol-7-yl]-1,2,4-triazin-3-yl]amino]-8-azabicyclo[3.2.1]octane-8-carboxylate (C137, 50.0 mg, 98%) as a solid. LCMS: (ES, m/z): 603.2 [M+H]⁺.

### Synthesis of Compound 543

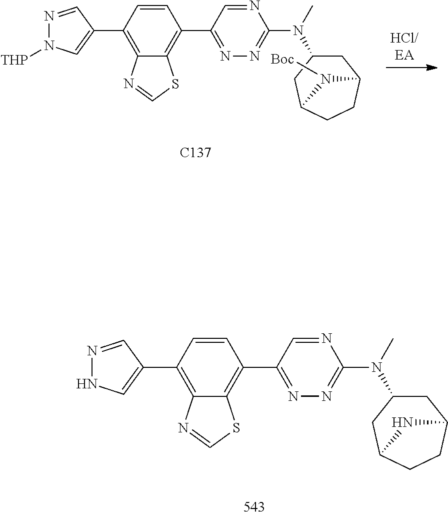

A solution of tert-butyl (1R,5S)-3-[methyl-[6-[4-(1-tetrahydropyran-2-ylpyrazol-4-yl)-1,3-benzothiazol-7-yl]-1,2,4-triazin-3-yl]amino]-8-azabicyclo[3.2.1]octane-8-carboxy late (C137, 50.0 mg, 83.0 μmol, 1 eq) in HCl (g)/ethyl acetate (4 M, 20.0 mL) was stirred at 20° C. for 1 hr. The mixture was concentrated to give a residue. The residue was purified by prep-HPLC (Condition 14, Gradient 9) to give (1R,5S)-N-methyl-N-[6-[4-(1H-pyrazol-4-yl)-1,3-benzothiazol-7-yl]-1,2,4-triazin-3-yl]-8-azabicyclo[3.2.1]octan-3-amine (Compound 543, 5.53 mg, 15%) as a solid. LCMS: (ES, m/z): 419.1 [M+H]⁺¹H NMR (400 MHz, METHANOL-d₄) 6 ppm 9.42 (s, 1H) 8.92 (br s, 2H) 8.06-8.11 (m, 1H) 7.96-8.04 (m, 1H) 6.08 (s, 1H) 4.39-4.55 (m, 1H) 4.24 (br s, 2H) 3.20 (s, 3H) 2.51-2.63 (m, 1H) 2.35-2.42 (m, 2H) 2.07-2.25 (m, 5H).

### Example 136: Synthesis of Compound 544

### Synthesis of Intermediate C138

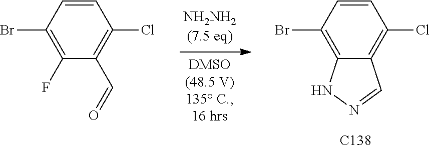 [SMILES: O=Cc1c(Cl)ccc(Br)c1F ; Clc1ccc(Br)c2[nH]ncc12 ; "C138"]

To a solution of 3-bromo-6-chloro-2-fluoro-benzaldehyde (20.0 g, 84.0 mmol, 1.0 eq) in dimethyl sulfoxide (970 mL) was added hydrazine hydrate (31.6 g, 630 mmol, 7.5 eq). The mixture was stirred at 135° C. for 16 hrs. TLC (petroleum ether/ethyl acetate=20/1, Rf=0.5) showed that the starting material was consumed and a new major spot was generated. After cooling to 20° C., the reaction mixture was quenched with water (1.50 L) at 5° C. and extracted with ethyl acetate (3×500 mL). The combined organic layer was washed with brine (500 mL), dried over Na₂SO₄, filtered and the filtrate was concentrated under reduced pressure to give crude product, which was purified by column chromatography on silica gel (petroleum ether/ethyl acetate=99:1) to give 7-bromo-4-chloro-1H-indazole (C138, 20.0 g, 94%) as a solid. LCMS: (ES, m/z): 230.9 [M+H]⁺, ¹H NMR (400 MHz, DMSO-d₆) δ 8.29 (br s, 1H), 7.60 (d, 1H, J=7.9 Hz), 7.16 (d, 1H, J=7.9 Hz)

### Synthesis of Intermediate C139

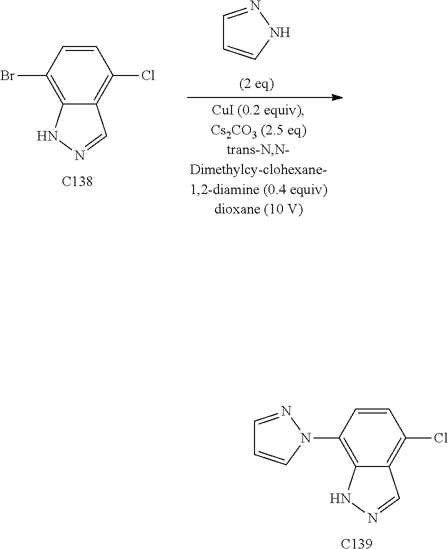 [SMILES: Clc1ccc(Br)c2[nH]ncc12 ; c1cn[nH]c1 ; "C138" ; Clc1ccc(-n2cccn2)c2[nH]ncc12 ; "C139"]

A mixture of 7-bromo-4-chloro-1H-indazole (1.00 g, 4.32 mmol, 1 eq), cesium carbonate (3.52 g, 10.8 mmol, 2.5 eq), 1H-pyrazole (58.9 mg, 8.63 mmol, 2 eq) and N1,N2-dimethylcyclohexane-1,2-diamine (24.6 mg, 1.73 mmol, 0.4 eq) in dioxane (10 mL) was added copper iodide (16.5 mg, 863 μmol, 0.2 eq) in glovebox at Ar atmosphere. The mixture was stirred at 100° C. for 16 hrs under Ar atmosphere. TLC (petroleum ether/ethyl acetate=5/1, Rf=0.6) showed that most of the starting material was consumed and a new major spot was generated. After cooling to 20° C., the reaction mixture was poured into water (500 mL) and extracted with CH₂Cl₂ (3×200 mL). The organic layer was dried over Na₂SO₄, filtered and the filtrate was concentrated under reduced pressure to give crude product, which was purified by column chromatography on silica gel (petroleum ether/ethyl acetate=24:1) to give 4-chloro-7-pyrazol-1-yl-1H-indazole (C139, 6.00 g, 33%) as a solid. LCMS: (ES, m/z): 219.1 [M+H]⁺, ¹H NMR (400 MHz, DMSO-d₆) δ 8.68 (br s, 1H), 8.27 (br d, 1H, J=1.1 Hz), 7.89 (d, 1H, J=1.5 Hz), 7.76 (d, 1H, J=8.1 Hz), 7.30 (d, 1H, J=8.0 Hz), 6.6-6.7 (m, 1H).

### Synthesis of Intermediate C140

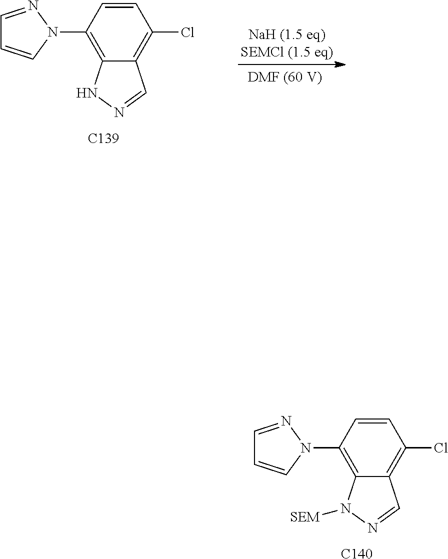 [SMILES: Clc1ccc(-n2cccn2)c2[nH]ncc12 ; "C139" ; C[Si](C)(C)CCOCn1ncc2c(Cl)ccc(-n3cccn3)c21 ; "C140"]

To a solution of 4-chloro-7-pyrazol-1-yl-1H-indazole (C139, 4.50 g, 20.6 mmol, 1 eq) in N,N-dimethylformamide (270 mL) was added sodium hydride (60 wt %, 741 mg, 30.9 mmol, 1.5 eq) at 0° C. in portions. After completion of addition, the mixture was stirred at 0° C. for 30 mins. Then (2-(chloromethoxy)ethyl)trimethylsilane (5.15 g, 30.9 mmol, 1.5 eq) was added dropwise at 0° C. The resulting mixture was stirred at 25° C. for additional 2 hrs. The reaction mixture was quenched with ice water (1.20 L) and extracted with ethyl acetate (3×400 mL). The organic layer was dried over Na₂SO₄, filtered and the filtrate was concentrated under reduced pressure to give crude product, which was purified by column chromatography on silica gel (petroleum ether/ethyl acetate=23/1) to give 2-[(4-chloro-7-pyrazol-1-yl-indazol-1-yl)methoxy]ethyl-trimethyl-silane (C140, 2.90 g, 40%) as a solid. LCMS: (ES, m/z): 348.9 [M+H]⁺, ¹HNMR (400 MHz, DMSO-d₆) δ 9.10 (d, 1H, J=2.4 Hz), 8.88 (s, 1H), 7.8-7.9 (m, 2H), 7.29 (d, 1H, J=7.9 Hz), 6.6-6.6 (m, 1H), 5.84 (s, 2H), 3.7-3.8 (m, 2H), 0.8-0.9 (m, 2H), -0.1-0.0 (m, 9H).

### Synthesis of Intermediate C141

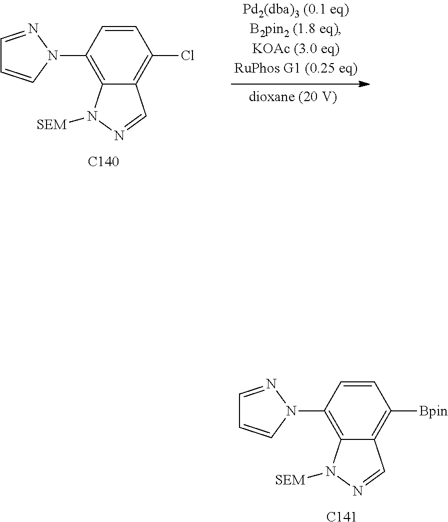

A mixture of 2-[(4-chloro-7-pyrazol-1-yl-indazol-1-yl)methoxy]ethyl-trimethyl-silane (C140, 2.90 g, 8.32 mmol, 1 eq), dicyclohexyl-[2-(2,6-diisopropoxyphenyl)phenyl] phosphane (969 mg, 2.08 mmol, 0.25 eq), potassium acetate (2.45 g, 24.9 mmol, 3 eq), bis(pinacolato)diboron (3.79 g, 14.9 mmol, 1.8 eq) and tris(dibenzylideneacetone) dipalladium (761 mg, 831 μmol, 0.1 eq) in dioxane (58 mL) was degassed and purged with N₂ three times. The mixture was stirred at 80° C. for 12 hrs under N₂ atmosphere. After cooling to 20° C., the reaction mixture was filtered and the filtrate was concentrated under reduced pressure to give crude product, which was purified by column chromatography on silica gel (petroleum ether/ethyl acetate=24:1) to give trimethyl-[2-[[7-pyrazol-1-yl-4-(4,4,5,5-tetramethyl-1,3,2-dioxaborolan-2-yl)indazol-1-yl]methoxy]ethyl]silane (C141, 1.70 g, 46%) as a solid. LCMS: (ES, m/z): 441.2 [M+H]⁺, ¹H NMR (400 MHz, DMSO-d₆) δ ppm 9.27 (d, J=2.25 Hz, 1H) 8.69 (s, 1H) 7.94 (d, J=7.38 Hz, 1H) 7.85 (d, J=1.38 Hz, 1H) 7.63 (d, J=7.38 Hz, 1H) 6.60-6.67 (m, 1H) 5.89 (s, 2H) 3.63-3.75 (m, 2H) 1.17 (s, 12H) 0.87-0.92 (m, 2H)-0.05 (s, 9H).

### Synthesis of Intermediate C142

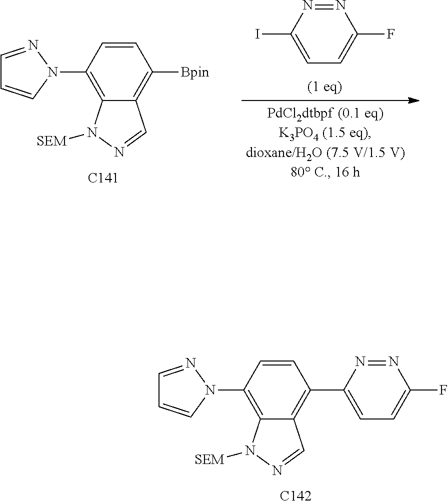

A mixture of trimethyl-[2-[[7-pyrazol-1-yl-4-(4,4,5,5-tetramethyl-1,3,2-dioxaborolan-2-yl)indazol-1-yl]methoxy]ethyl]silane (A1-5, 800 mg, 1.82 mmol, 1 eq), 3-fluoro-6-iodo-pyridazine (C141, 407 mg, 1.82 mmol, 1 eq), ditert-butyl (cyclopentyl) phosphane; dichloropalladium; iron (118 mg, 182 μmol, 0.1 eq), K₃PO₄ (578 mg, 2.72 mmol, 1.5 eq) in a mixture solvents of dioxane (6 mL) and H₂O (1.2 mL) was degassed and purged with N₂ for 3 times. The mixture was stirred at 80° C. for 16 hrs under N₂ atmosphere. After cooling to 20° C., the reaction mixture was poured into water (30 mL) at 25° C. and extracted with dichloromethane (3×10 mL). The organic layer was dried over Na₂SO₄, filtered and the filtrate was concentrated under reduced pressure to give crude product, which was purified by column chromatography on silica gel (petroleum ether/ethyl acetate=24:1) to give 2-[[4-(6-fluoropyridazin-3-yl)-7-pyrazol-1-yl-indazol-1-yl]methoxy]ethyl-trimethyl-silane (C142, 200 mg, 15%) as an oil. LCMS: (ES, m/z): 411.2 [M+H]⁺, ¹H NMR (400 MHz, DMSO-d₆) δ ppm 9.22-9.36 (m, 2H) 8.68 (dd, J=9.42, 7.39 Hz, 1H) 7.98-8.09 (m, 2H) 7.84-7.93 (m, 2H) 6.64-6.68 (m, 1H) 3.92 (s, 2H) 3.73 (t, J=8.05 Hz, 2H) 0.90 (br t, J=7.99 Hz, 2H) -0.06 (s, 9H).

### Synthesis of Intermediate C143

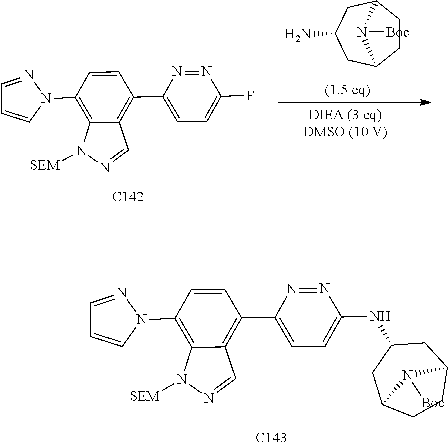

A mixture of 2-[[4-(6-fluoropyridazin-3-yl)-7-pyrazol-1-yl-indazol-1-yl]methoxy] ethyl-trimethyl-silane (C142, 20.0 mg, 48.7 μmol, 1 eq), tert-butyl (1S,5R)-3-amino-8-azabicyclo[3.2.1]octane-8-carboxylate (16.5 mg, 73.1 μmol, 1.5 eq), N,N-diisopropylethylamine (18.9 mg, 146 μmol, 3 eq) in dimethyl sulfoxide (0.2 mL) was stirred at 130° C. for 16 hrs under N₂ atmosphere. After cooling to 20° C., the reaction mixture was quenched with water (5 mL) at 25° C. and extracted with dichloromethane (5×4 mL). The organic layer was dried over Na₂SO₄, filtered and the filtrate was concentrated under reduced pressure to give crude product, which was purified by column chromatography on silica gel (petroleum ether/ethyl acetate=4/1) to give tert-butyl (1R,5S)-3-[[6-[7-pyrazol-1-yl-1-(2-trimethylsilylethoxymethyl)indazol-4-yl]pyridazin-3-yl]amino]-8-azabicyclo[3.2.1]octane-8-carboxylate (C143, 20.0 mg, 67%) as a solid. LCMS: (ES, m/z): 617.4 [M+H]⁺. ¹H NMR (400 MHz, DMSO-d₆) δ ppm 9.23-9.36 (m, 2H) 7.62-8.02 (m, 3H) 6.83-7.09 (m, 2H) 6.60-6.69 (m, 1H) 5.71-5.96 (m, 2H) 4.54-4.66 (m, 1H) 4.14 (br s, 2H) 3.69-3.75 (m, 2H) 1.93-2.06 (m, 4H) 1.74-1.85 (m, 2H) 1.41-1.57 (m, 11H) 0.80-0.97 (m, 2H)-0.06 (br s, 9H).

### Synthesis of Intermediate C144

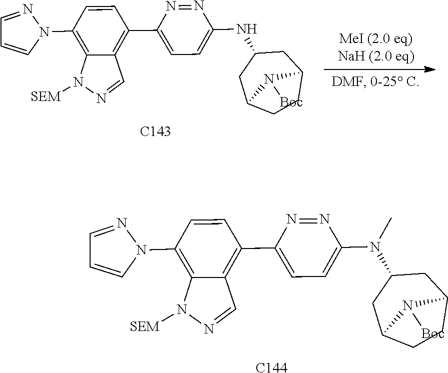 [SMILES: CC(C)(C)OC(=O)N1[C@@H]2CC[C@H]1C[C@H](Nc1ccc(-c3ccc(-n4cccn4)c4c3cnn4COCC[Si](C)(C)C)nn1)C2 ; "C143" ; CN(c1ccc(-c2ccc(-n3cccn3)c3c2cnn3COCC[Si](C)(C)C)nn1)[C@@H]1C[C@H]2CC[C@@H](C1)N2C(=O)OC(C)(C)C ; "C144"]

To a solution of tert-butyl (1R,5S)-3-[[6-[7-pyrazol-1-yl-1-(2-trimethylsilylethoxy methyl)indazol-4-yl]pyridazin-3-yl]amino]-8-azabicyclo[3.2.1]-octane-8-carboxylate (C143, 20.0 mg, 32.4 μmol, 1 eq) in N,N-dimethylformamide (0.200 mL) was added sodium hydride (60 wt %, 5.19 mg, 64.8 μmol, 2 eq) at 0° C. in portions. After completion of addition, the mixture was stirred at 0° C. for 30 mins. Then iodomethane (5.98 mg, 42.1 μmol, 2 eq) was added at 0° C. The resulting mixture was stirred at 20° C. for 12 hrs. The reaction mixture was quenched with H₂O (20.0 mL) and extracted with dichloromethane (5×10 mL). The organic layer was dried over Na₂SO₄, filtered and the filtrate was concentrated under reduced pressure to give crude tert-butyl (1R,5S)-3-[methyl-[6-[7-pyrazol-1-yl-1-(2-trimethylsilylethoxy methyl)indazol-4-yl]pyridazin-3-yl]amino]-8-azabicyclo[3.2.1]octane-8-carboxylate (C144, 20.0 mg, 78%) as a solid LCMS: (ES, m/z): 631.3 [M+H]⁺.

### Synthesis of Compound 544

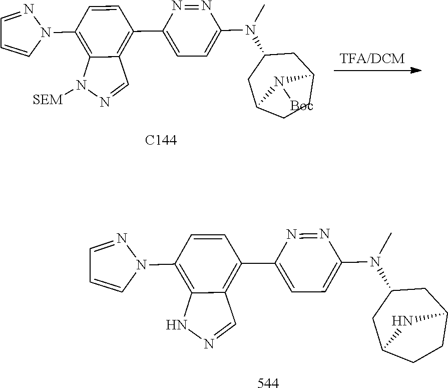 [SMILES: CN(c1ccc(-c2ccc(-n3cccn3)c3c2cnn3COCC[Si](C)(C)C)nn1)[C@@H]1C[C@H]2CC[C@@H](C1)N2C(=O)OC(C)(C)C ; "C144" ; CN(c1ccc(-c2ccc(-n3cccn3)c3[nH]ncc23)nn1)[C@@H]1C[C@H]2CC[C@@H](C1)N2 ; "544"]

To a solution of tert-butyl (1R,5S)-3-[methyl-[6-[7-pyrazol-1-yl-1-(2-trimethylsilylethoxymethyl)indazol-4-yl]pyridazin-3-yl]amino]-8-azabicyclo[3.2.1]-octane-8-carboxylate (C144, 20.0 mg, 31.7 μmol, 1 eq) in dichloro-methane (0.30 mL) was added trifluoroacetic acid (0.10 mL) at 25° C. The resulting mixture was stirred at 25° C. for 16 hrs. The reaction mixture was concentrated under reduced pressure to give a residue. The residue was purified by prep-HPLC (Condition 14, Gradient 5) to give (1R,5S)-N-methyl-N-[6-(7-pyrazol-1-yl-1H-indazol-4-yl)pyridazin-3-yl]-8-azabicyclo[3.2.1]octan-3-amine (Compound 544, 7.00 mg, 55%) as a solid. LCMS: (ES, m/z): 401.2 [M+H]⁺, ¹H NMR (400 MHz, METHANOL-d₄) δ ppm 8.74 (s, 1H) 8.57-8.64 (m, 2H) 8.20 (br d, J=9.90 Hz, 1H) 7.96 (d, J=1.59 Hz, 1H) 7.86-7.91 (m, 2H) 6.68 (t, J=2.14 Hz, 1H) 4.75-4.81 (m, 1H) 4.26 (br s, 2H) 3.29 (s, 3H) 2.31-2.43 (m, 4H) 2.22-2.29 (m, 2H) 2.09-2.15 (m, 2H).

### Example 137: Synthesis of Compound 545

### Synthesis of Intermediate C145

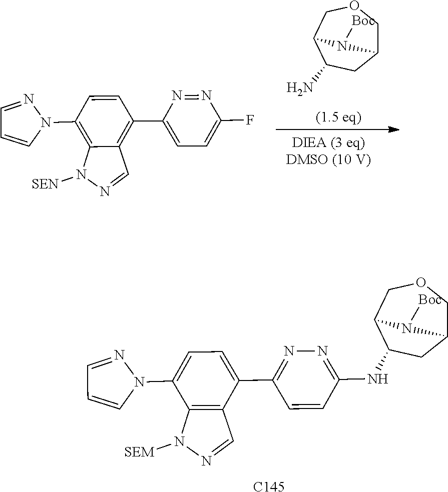

A mixture of 2-[[4-(6-fluoropyridazin-3-yl)-7-pyrazol-1-yl-indazol-1-yl]methoxy] ethyl-trimethyl-silane (85.0 mg, 207 μmol, 1 eq), tert-butyl (1S,5R)-7-amino-3-oxa-9-azabicyclo[3.3.1]nonane-9-carboxylate (C8, 80.2 mg, 621 μmol, 1.5 eq), N,N-diisopropylethylamine (75.2 mg, 310 μmol, 3 eq) in dimethyl sulfoxide (0.85 mL) was stirred at 130° C. for 16 hrs. The reaction mixture was quenched with H₂O (20 mL) at 5° C. The reaction mixture was extracted with dichloromethane (5×10 mL). The combined organic layer was dried over Na₂SO₄, filtered and the filtrate was concentrated under reduced pressure to give crude product. The residue was purified by column chromatography (SiO₂, 18% of ethyl acetate in petroleum ether) to give tert-butyl (1S,5R)-7-[[6-[7-pyrazol-1-yl-1-(2-trimethylsilylethoxymethyl) indazol-4-yl]pyridazin-3-yl]amino]-3-oxa-9-azabicyclo[3.3.1]nonane-9-carboxylate (C145, 50.0 mg, 38%) as an oil. LCMS. (ES, m/z): 633.4 [M+H]⁺. 1H NMR (400 MHz, DMSO-d₆) δ ppm 9.21-9.34 (m, 2H) 7.98 (dd, J=8.62, 3.85 Hz, 2H) 7.85 (s, 1H) 7.69 (d, J=7.58 Hz, 1H) 6.80-6.91 (m, 2H) 6.62 (s, 1H) 5.91 (s, 2H) 5.25-5.38 (m, 1H) 3.96-4.06 (m, 2H) 3.87-3.95 (m, 2H) 3.72 (t, J=7.76 Hz, 2H) 3.63 (br d, J=11.00 Hz, 2H) 1.54-1.61 (m, 2H) 1.42-1.48 (m, 9H) 0.78-0.95 (m, 4H)-0.06 (s, 9 H).

### Synthesis of Intermediate C146

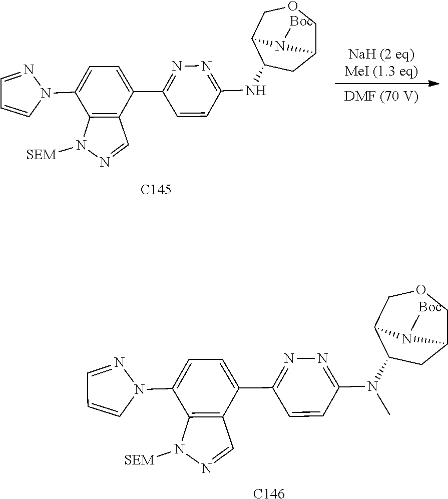 [SMILES: CC(C)(C)OC(=O)N1[C@H]2COC[C@@H]1[C@@H](Nc1ccc(-c3ccc(-n4cccn4)c4c3cnn4COCC[Si](C)(C)C)nn1)C2 ; "C145" ; CN(c1ccc(-c2ccc(-n3cccn3)c3c2cnn3COCC[Si](C)(C)C)nn1)[C@H]1C[C@@H]2COC[C@H]1N2C(=O)OC(C)(C)C ; "C146"]

To a solution of tert-butyl (1S,5R)-7-[[6-[7-pyrazol-1-yl-1-(2-trimethylsilylethoxy methyl)indazol-4-yl]pyridazin-3-yl]amino]-3-oxa-9-azabicyclo-[3.3.1]nonane-9-carboxylate (C145, 50.0 mg, 79.0 μmol, 1 eq) in N,N-dimethylformamide (3.50 mL) was added sodium hydride (60 wt %, 6.32 mg, 158 μmol, 2 eq) at 0° C. After completion of addition, the mixture was stirred at 0° C. for 30 mins. Then iodomethane (14.6 mg, 102 μmol, 1.3 eq) was added dropwise at 0° C. The resulting mixture was stirred for additional 12 hrs. The reaction mixture was quenched with H₂O (20 mL) at 5° C., and extracted with dichloromethane (5×10 mL). The organic layer was dried over Na₂SO₄, filtered and the filtrate was concentrated under reduced pressure to give crude tert-butyl (1S,5R)-7-[methyl-[6-[7-pyrazol-1-yl-1-(2-trimethylsilylethoxymethyl)indazol-4-yl]pyridazin-3-yl]amino]-3-oxa-9-azabicyclo [3.3.1]nonane-9-carboxylate (C146, 30.0 mg, 58%) as an oil. The crude product was used in the next step directly without further purification. LCMS: (ES, m/z): 647.3 [M+H]⁺.

### Synthesis of Compound 545

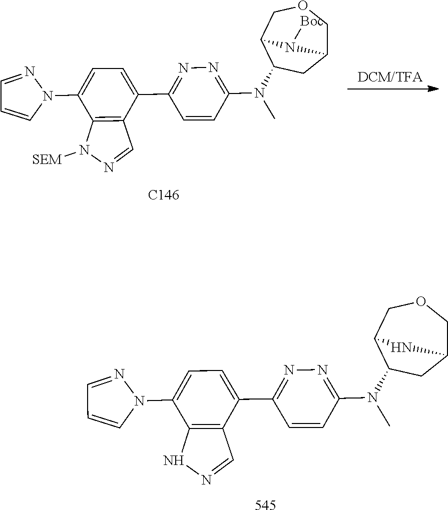 [SMILES: CN(c1ccc(-c2ccc(-n3cccn3)c3c2cnn3COCC[Si](C)(C)C)nn1)[C@H]1C[C@@H]2COC[C@H]1N2C(=O)OC(C)(C)C ; "C146" ; CN(c1ccc(-c2ccc(-n3cccn3)c3[nH]ncc23)nn1)[C@H]1C[C@@H]2COC[C@H]1N2 ; "545"]

To a solution of tert-butyl (1S,5R)-7-[methyl-[6-[7-pyrazol-1-yl-1-(2-trimethylsilyl ethoxymethyl)indazol-4-yl]pyridazin-3-yl]amino]-3-oxa-9-azabicyclo-[3.3.1]nonane-9-carboxylate (C146, 30.0 mg, 46.4 μmol, 1 eq) in dichloromethane (0.150 mL) was added dropwise trifluoroacetic acid (46.2 mg, 405 μmol, 8.52 eq). The resulting mixture was stirred at 2 hrs. The reaction mixture was concentrated under reduced pressure to give a residue. The residue was purified by prep-HPLC (Condition 14, Gradient 4) to give ((1S,5R)—N-methyl-N-[6-(7-pyrazol-1-yl-1H-indazol-4-yl)pyridazin-3-yl]-3-oxa-9-azabicyclo[3.3.1]nonan-7-amine (Compound 545, 5.78 mg, 30%) as a solid. LCMS: (ES, m/z): 417.2 [M+H]⁺. ¹H NMR (400 MHz, METHANOL-d₄) δ ppm 8.73 (s, 1H) 8.46-8.50 (m, 1H) 8.04 (d, J=9.66 Hz, 1H) 7.93 (s, 1H) 7.79 (d, J=7.75 Hz, 1H) 7.65 (d, J=7.87 Hz, 1H) 7.30 (d, J=9.66 Hz, 1H) 6.64 (t, J=2.21 Hz, 1H) 6.02-6.15 (m, 1H) 4.13-4.18 (m, 2H) 3.97-4.06 (m, 2H) 3.56 (br s, 2H) 3.09 (s, 3H) 2.36-2.51 (m, 2H) 2.20 (br dd, J=14.60, 5.19 Hz, 2H)

### Example 137: Synthesis of Compound 479

### Synthesis of Intermediate C147

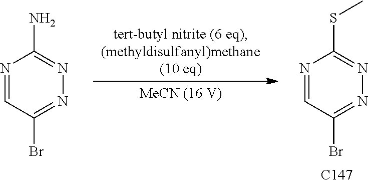

To a solution of 6-bromo-1,2,4-triazin-3-amine (10.0 g, 57.2 mmol, 1 eq) in CH₃CN (160 mL) was added (methyldisulfanyl)methane (53.8 g, 571 mmol, 51.3 mL, 10 eq) dropwise at 20° C. Then tert-butyl nitrite (35.4 g, 343 mmol, 40.8 mL, 6 eq) was added dropwise at 20° C. The resulting mixture was stirred at 30° C. for 1 hr. TLC (petroleum ether/ethyl acetate=5/1, Rf=0.5) showed that the starting material was consumed and a new major spot was generated. The reaction mixture was filtered and the filtrate was quenched by methanol (300 mL) at 20° C. The resulting mixture was concentrated under reduced pressure to give a residue. The residue was purified by column chromatography (SiO₂, 5% of ethyl acetate in petroleum ether) to give 6-bromo-3-methylsulfanyl-1,2,4-triazine (C147, 4.00 g, 33%) as a solid. LCMS: (ES, m/z): 205.9 [M+H]⁺. 1H NMR (400 MHz, DMSO-d₆) δ ppm 8.88 (s, 1H) 2.61 (s, 3H).

### Synthesis of Intermediate C149

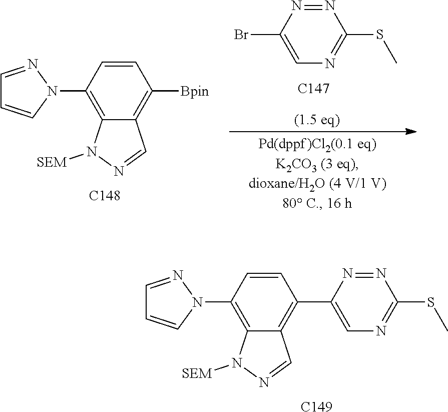

A mixture of trimethyl-[2-[[7-pyrazol-1-yl-4-(4,4,5,5-tetramethyl-1,3,2-dioxaborolan-2-yl)indazol-1-yl]methoxy]ethyl]silane (C148, 1.40 g, 3.18 mmol, 1 eq), 6-bromo-3-methylsulfanyl-1,2,4-triazine (C147, 982 mg, 4.77 mmol, 1.5 eq), K₂CO₃ (1.32 g, 9.54 mmol, 3 eq), 1,1′-bis(diphenylphosphino)ferrocenedichloro palladium(II) (233 mg, 318 μmol, 0.1 eq) in dioxane (5.60 mL) and H₂O (1.40 mL) was degassed and purged with N₂ for 3 times. The mixture was stirred at 80° C. for 16 hrs under N₂ atmosphere. After cooling to 20° C., the reaction mixture was filtered and the filter cake was washed with dichloromethane (3×10 mL). H₂O (30 mL) was added to the mixture and the mixture was extracted with dichloromethane (3×10 mL). The combined organic layers were dried over Na₂SO₄, filtered and concentrated under reduced pressure to give a residue. The residue was purified by column chromatography (SiO₂, 12% of ethyl acetate in petroleum ether) to give trimethyl-[2-[[4-(3-methylsulfanyl-1,2,4-triazin-6-yl)-7-pyrazol-1-yl-indazol-1 yl]methoxy]ethyl] silane (C150, 800 mg, 57%) was as a solid. LCMS: (ES, m/z): 440.1 [M+H]⁺. ¹HNMR (400 MHz, DMSO-d₆) δ ppm 9.42 (s, 1H) 9.27-9.34 (m, 2H) 8.04-8.13 (m, 2H) 7.91 (d, J=1.47 Hz, 1H) 6.64-6.69 (m, 1H) 5.94 (s, 2H) 3.69-3.77 (m, 2H) 2.72 (s, 3H) 0.86-0.94 (m, 2H)-0.06 (s, 9H).

### Synthesis of Intermediate C151

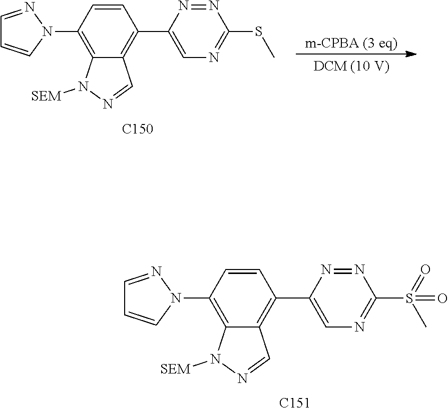 [SMILES: CSc1ncc(-c2ccc(-n3cccn3)c3c2cnn3COCC[Si](C)(C)C)nn1 ; "C150" ; C[Si](C)(C)CCOCn1ncc2c(-c3cnc(S(C)(=O)=O)nn3)ccc(-n3cccn3)c21 ; "C151"]

To a solution of trimethyl-[2-[[4-(3-methylsulfanyl-1,2,4-triazin-6-yl)-7-pyrazol-1-yl-indazol-1-yl]methoxy]ethyl]silane (C150, 800 mg, 1.82 mmol, 1 eq) in dichloromethane (8.00 mL) was added 3-chloroperoxybenzoic acid (85 wt %, 1.11 g, 5.46 mmol, 3 eq) at 0° C. The mixture was stirred at 20° C. for 2 hrs. The reaction mixture was quenched by addition of saturated NaHCO₃ solution (30 mL) at 0° C., and the mixture was extracted with dichloromethane (3×10 mL). The combined organic layers were dried over Na₂SO₄, filtered and concentrated under reduced pressure to give a residue. The crude trimethyl-[2-[[4-(3-methylsulfonyl-1,2,4-triazin-6-yl)-7-pyrazol-1-yl-indazol-1-yl]methoxy]ethyl]silane (C151, 800 mg, 51%) was used directly into the next step without further purification. LCMS: (ES, m/z): 472.1 [M+H]⁺. ¹HNMR (400 MHz, DMSO-d₆) δ ppm 9.88 (s, 1H) 9.29-9.42 (m, 2H) 8.33-8.40 (m, 1H) 8.13-8.18 (m, 1H) 7.95 (s, 1H) 6.70 (br s, 1H) 5.96 (s, 2H) 3.74 (brt, J=8.13 Hz, 2H) 3.58 (s, 3H) 0.88-0.93 (m, 2H)-0.06 (s, 9H).

### Synthesis of Intermediate C153

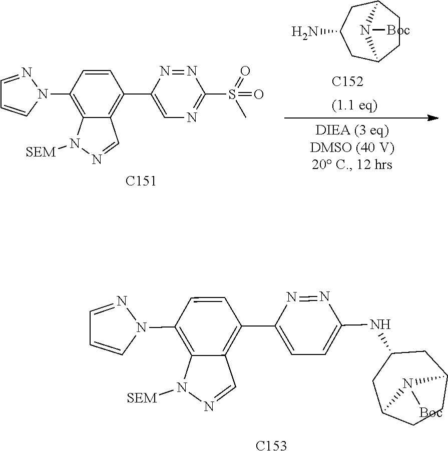 [SMILES: CC(C)(C)OC(=O)N1[C@@H]2CC[C@H]1C[C@H](N)C2 ; "C152" ; C[Si](C)(C)CCOCn1ncc2c(-c3cnc(S(C)(=O)=O)nn3)ccc(-n3cccn3)c21 ; "C151" ; CC(C)(C)OC(=O)N1[C@@H]2CC[C@H]1C[C@H](Nc1ccc(-c3ccc(-n4cccn4)c4c3cnn4COCC[Si](C)(C)C)nn1)C2 ; "C153"]

A mixture of trimethyl-[2-[[4-(3-methylsulfonyl-1,2,4-triazin-6-yl)-7-pyrazol-1-yl-indazol-1-yl]methoxy]ethyl]silane (C151, 160 mg, 339 μmol, 1 eq), tert-butyl (1R,5S)-3-amino-8-azabicyclo[3.2.1]octane-8-carboxylate (C152, 84.5 mg, 373 μmol, 1.1 eq), N,N-diisopropylethylamine (132 mg, 1.02 mmol, 3 eq) in dimethyl sulfoxide (6.40 mL) was stirred at 20° C. for 12 hrs. The reaction mixture was quenched with water (30 mL) at 20° C. and the reaction mixture was extracted with ethyl acetate (5×10 mL). The organic layer was dried over Na₂SO₄, filtered and the filtrate was concentrated under reduced pressure to give crude product, which was purified by column chromatography on silica gel (SiO₂, 29% of ethyl acetate in petroleum ether) to give tert-butyl (1R,5S)-3-[[6-[7-pyrazol-1-yl-1-(2-trimethylsilylethoxymethyl)indazol-4-yl]-1,2,4-triazin-3-yl]amino]-8-azabicyclo[3.2.1]octane-8-carboxylate (C153, 80.0 mg, 38%) as a solid. LCMS: (ES, m/z): 618.1 [M+H]⁺. ¹HNMR (400 MHz, DMSO-d₆) δ ppm 9.19-9.27 (m, 2H) 9.04 (s, 1H) 7.99 (d, J=7.75 Hz, 1 H) 7.79-7.89 (m, 2H) 6.61-6.67 (m, 1H) 5.91 (s, 2H) 4.28-4.71 (m, 1H) 4.14 (br s, 2H) 3.72 (t, J=8.05 Hz, 2H) 2.52-2.57 (m, 2H) 1.94 (br d, J=0.72 Hz, 2H) 1.63-1.83 (m, 4H) 1.45 (s, 9H) 0.86-0.95 (m, 2H)-0.06 (s, 9H).

### Synthesis of Intermediate C154

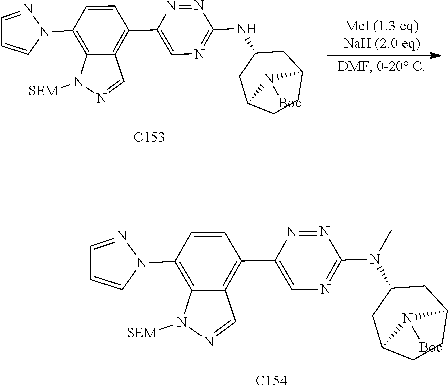 [SMILES: CC(C)(C)OC(=O)N1[C@@H]2CC[C@H]1C[C@H](Nc1ncc(-c3ccc(-n4cccn4)c4c3cnn4COCC[Si](C)(C)C)nn1)C2 ; "C153" ; CN(c1ncc(-c2ccc(-n3cccn3)c3c2cnn3COCC[Si](C)(C)C)nn1)[C@@H]1C[C@H]2CC[C@@H](C1)N2C(=O)OC(C)(C)C ; "C154"]

To a solution of tert-butyl (1R,5S)-3-[[6-[7-pyrazol-1-yl-1-(2-trimethylsilyl ethoxymethyl)indazol-4-yl]-1,2,4-triazin-3-yl]amino]-8-azabicyclo[3.2.1]octane-8-carboxylate (C153, 50.0 mg, 80.9 μmol, 1 eq) in N,N-dimethylformamide (0.500 mL) was added sodium hydride (60 wt %, 12.9 mg, 162 μmol, 2 eq) at 0° C. After completion of addition, the mixture was stirred at 20° C. for 30 min. Then iodomethane (14.9 mg, 105 μmol, 1.3 eq) was added dropwise at 0° C. The resulting mixture was stirred at 20° C. for 12 hrs. The reaction mixture was quenched with H₂O (20.0 mL) and extracted with dichloromethane (5×10 mL). The organic layer was dried over Na₂SO₄, filtered and the filtrate was concentrated under reduced pressure to give crude tert-butyl (1R, 5S)-3-[methyl-[6-[7-pyrazol-1-yl-1-(2-trimethylsilylethoxymethyl) indazol-4-yl]-1,2,4-tri azin-3-yl] amino]-8-azabicyclo [3.2.1]octane-8-carboxylate (C154, 30.0 mg, 59%) as a solid. The crude product was used in the next step directly without further purification. LCMS: (ES, m/z): 632.2 [M+H]⁺.

### Synthesis of Compound 479

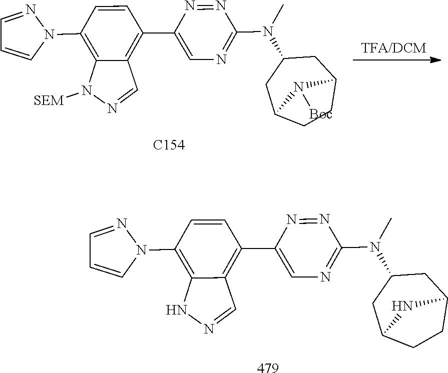 [SMILES: CN(c1ncc(-c2ccc(-n3cccn3)c3c2cnn3COCC[Si](C)(C)C)nn1)[C@@H]1C[C@H]2CC[C@@H](C1)N2C(=O)OC(C)(C)C ; "C154" ; CN(c1ncc(-c2ccc(-n3cccn3)c3[nH]ncc23)nn1)[C@@H]1C[C@H]2CC[C@@H](C1)N2 ; "479"]

To a solution of tert-butyl (1R,5S)-3-[methyl-[6-[7-pyrazol-1-yl-1-(2-trimethylsilyl ethoxymethyl)indazol-4-yl]-1,2,4-triazin-3-yl]amino]-8-azabicyclo[3.2.1]octane-8-carboxylate (C154, 25.0 mg, 39.6 μmol, 1 eq) in dichloromethane (0.375 mL) was added trifluoroacetic acid (0.125 mL) dropwise at 0° C. The resulting mixture was stirred at 20° C. for 12 hrs. The reaction mixture was concentrated under reduced pressure to give a residue. The residue was purified by prep-HPLC (Condition 14, Gradient 10) to give (1R,5S)-N-methyl-N-[6-(7-pyrazol-1-yl-1H-indazol-4-yl)-1,2,4-triazin-3-yl]-8-azabicyclo[3.2.1]octan-3-amine (14.7 mg, 62.9%, HCl salt) as a solid. LCMS: (ES, m/z): 402.2 [M+H]⁺. ¹HNMR (400 MHz, DMSO-d₆) δ ppm 9.07 (s, 1H) 8.64-8.77 (m, 2H) 7.93 (s, 1H) 7.87 (d, J=7.99 Hz, 1H) 7.77 (d, J=7.87 Hz, 1H) 6.67 (d, J=1.79 Hz, 1H) 5.15-5.31 (m, 1H) 4.12 (br s, 2H) 3.14 (s, 3H) 2.23 (br t, J=11.62 Hz, 2H) 1.95-2.12 (m, 4H) 1.77-1.88 (m, 2H).

### Example 138: Synthesis of Compound 505

### Synthesis of Intermediate C155

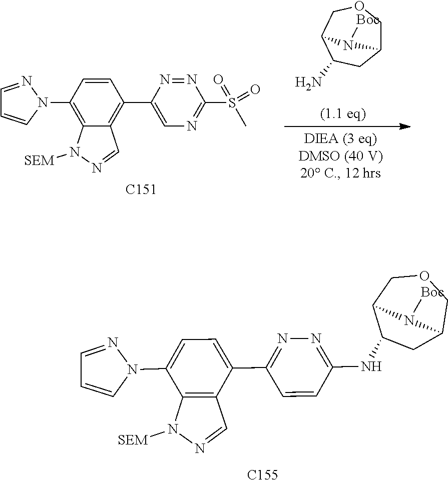 [SMILES: C[Si](C)(C)CCOCn1ncc2c(-c3cnc(S(C)(=O)=O)nn3)ccc(-n3cccn3)c21 ; CC(C)(C)OC(=O)N1[C@H]2COC[C@@H]1[C@@H](N)C2 ; "C151" ; CC(C)(C)OC(=O)N1[C@H]2COC[C@@H]1[C@@H](Nc1ccc(-c3ccc(-n4cccn4)c4c3cnn4COCC[Si](C)(C)C)nn1)C2 ; "C155"]

A mixture of trimethyl-[2-[[4-(3-methylsulfonyl-1,2,4-triazin-6-yl)-7-pyrazol-1-yl-indazol-1-yl]methoxy]ethyl]silane (C151, 150 mg, 318 μmol, 1 eq), tert-butyl (1R,5S)-3-amino-9-azabicyclo[3.3.1]nonane-9-carboxylate (84.1 mg, 350 μmol, 1.1 eq), N,N-diisopropylethylamine (123 mg, 954 μmol, 3 eq) in dimethyl sulfoxide (6 mL) was stirred at 20° C. for 12 hrs. The reaction mixture was quenched with water (30 mL) at 20° C. and extracted with ethyl acetate (5×10 mL). The organic layer was dried over Na₂SO₄, filtered and the filtrate was concentrated under reduced pressure to give crude product, which was purified by column chromatography on silica gel (SiO₂, 29% of ethyl acetate in petroleum ether) to give tert-butyl (1R,5S)-3-[[6-[7-pyrazol-1-yl-1-(2-trimethylsilylethoxymethyl) indazol-4-yl]-1,2,4-triazin-3-yl]amino]-9-azabicyclo[3.3.1]nonane-9-carboxylate (C151, 100 mg, 50%) as a solid. LCMS: (ES, m/z): 634.2 [M+H]⁺. 1HNMR (400 MHz, DMSO-d₆) δ ppm 9.25 (br d, J=2.45 Hz, 2H) 9.05 (s, 1H) 7.99 (d, J=7.83 Hz, 1H) 7.86 (d, J=1.10 Hz, 2H) 6.62-6.67 (m, 1H) 5.91 (s, 2H) 4.85-5.22 (m, 1H) 4.18-4.32 (m, 2H) 3.72 (br t, J=8.01 Hz, 2H) 2.02-2.14 (m, 2H) 1.72 (br d, J=1.34 Hz, 6H) 1.43 (s, 9H) 0.90 (br t, J=8.01 Hz, 2H)-0.06 (s, 9H).

### Synthesis of Intermediate C156

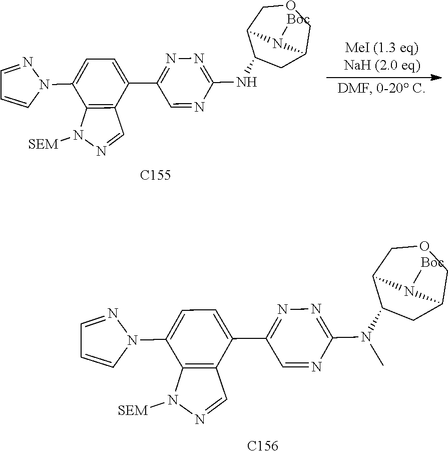 [SMILES: CC(C)(C)OC(=O)N1[C@H]2COC[C@@H]1[C@@H](Nc1ncc(-c3ccc(-n4cccn4)c4c3cnn4COCC[Si](C)(C)C)nn1)C2 ; "C155" ; CN(c1ncc(-c2ccc(-n3cccn3)c3c2cnn3COCC[Si](C)(C)C)nn1)[C@H]1C[C@@H]2COC[C@H]1N2C(=O)OC(C)(C)C ; "C156"]

To a solution of tert-butyl (1S,5R)-7-[[6-[7-pyrazol-1-yl-1-(2-trimethylsilylethoxy methyl)indazol-4-yl]-1,2,4-triazin-3-yl]amino]-3-oxa-9-azabicyclo[3.3.1] nonane-9-carboxylate (C155, 95.0 mg, 150 μmol, 1 eq) in N,N-dimethylformamide (0.500 mL) was added sodium hydride (60 wt %, 12.0 mg, 300 μmol, 2 eq) at 0° C. After completion of addition, the mixture was stirred at 20° C. for 30 min. Then iodomethane (28.0 mg, 195 μmol, 1.3 eq) was added dropwise at 0° C. The resulting mixture was stirred at 20° C. for 12 hrs. The reaction mixture was quenched with H₂O (20.0 mL) and the mixture was extracted with dichloromethane (5×10 mL). The organic layer was dried over Na₂SO₄, filtered and the filtrate was concentrated under reduced pressure to give crude tert-butyl (1S,5R)-7-[methyl-[6-[7-pyrazol-1-yl-1-(2-trimethylsilylethoxymethyl)indazol-4-yl]-1,2,4-triazin-3-yl]amino]-3-oxa-9-azabicyclo[3.3.1]nonane-9-carboxylate (C156, 40.0 mg, 41%) as a solid. The crude product was used in the next step directly without further purification. LCMS: (ES, m/z): 648.1 [M+H]⁺.

### Synthesis of Compound 505

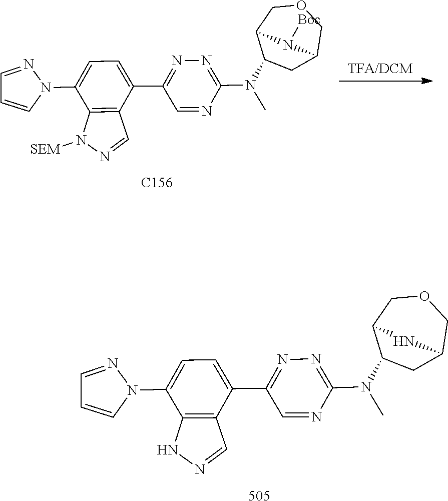 [SMILES: CN(c1ncc(-c2ccc(-n3cccn3)c3c2cnn3COCC[Si](C)(C)C)nn1)[C@H]1C[C@@H]2COC[C@H]1N2C(=O)OC(C)(C)C ; "C156" ; CN(c1ncc(-c2ccc(-n3cccn3)c3[nH]ncc23)nn1)[C@H]1C[C@@H]2COC[C@H]1N2 ; "505"]

To a mixture of tert-butyl(1S,5R)-7-[methyl-[6-[7-pyrazol-1-yl-1-(2-trimethylsilyl ethoxymethyl)indazol-4-yl]-1,2,4-triazin-3-yl] amino]-3-oxa-9-azabicyclo [3.3.1] nonane-9-carboxylate (C156, 35.0 mg, 55.3 μmol, 1 eq) in dichloromethane (0.525 mL) was added dropwise trifluoroacetate (0.175 mL) at 0° C. The resulting mixture was stirred at 20° C. for 12 hrs. The reaction mixture was concentrated under reduced pressure to give a residue, which was purified by prep-HPLC (Condition 14, Gradient 10) to give (1S,5R)—N-methyl-N-[6-(7-pyrazol-1-yl-1H-indazol-4-yl)-1,2,4-triazin-3-yl]-3-oxa-9-azabicyclo[3.3.1]nonan-7-amine (Compound 505, 11.3 mg, 43.8%, HCl salt) as yellow solid. LCMS: (ES, m/z): 418.2 [M+H]⁺. ¹HNMR (400 MHz, DMSO-d₆) δ ppm 9.10 (s, 1H) 8.66-8.80 (m, 2H) 7.94 (s, 1H) 7.77-7.91 (m, 2H) 6.68 (s, 1H) 6.03-6.23 (m, 1H) 4.03-4.10 (m, 2H) 3.92-4.00 (m, 2H) 3.70 (br s, 2H) 3.15 (s, 3H) 2.39 (br s, 2H) 2.03-2.10 (m, 2H).

### Example 139: Synthesis of Compound 546

### Synthesis of Intermediate C157

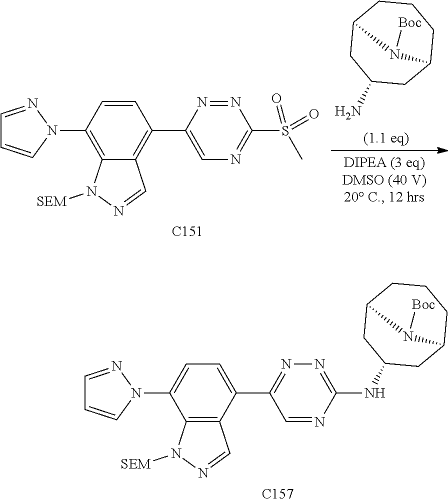

A mixture of trimethyl-[2-[[4-(3-methylsulfonyl-1,2,4-triazin-6-yl)-7-pyrazol-1-yl-indazol-1-yl]methoxy]ethyl]silane (C151, 150 mg, 318 μmol, 1 eq), tert-butyl (1R,5S)-3-amino-9-azabicyclo[3.3.1]nonane-9-carboxylate (C9, 84.1 mg, 350 μmol, 1.1 eq), N,N-diisopropylethylamine (123 mg, 954 μmol, 3 eq) in dimethyl sulfoxide (6 mL) was stirred at 20° C. for 12 hrs. The reaction mixture was quenched with water (30 mL) at 20° C. and extracted with ethyl acetate (5×10 mL). The combined organic layer was dried over Na₂SO₄, filtered and the filtrate was concentrated under reduced pressure to give crude product. The crude product was purified by column chromatography on silica gel (SiO₂, 29% of ethyl acetate in petroleum ether) to give tert-butyl (1R, 5S)-3-[[6-[7-pyrazol-1-yl-1-(2-trimethylsilylethoxymethyl)indazol-4-yl]-1,2,4-triazin-3-yl]amino]-9-azabicyclo[3.3.1]nonane-9-carboxylate (C157, 100 mg, 50%) as a solid. LCMS: (ES, m/z): 632.4 [M+H]⁺. ¹HNMR (400 MHz, DMSO-d₆) δ ppm 9.25 (d, J=2.27 Hz, 2H) 9.05 (s, 1H) 7.99 (d, J=7.87 Hz, 1H) 7.79-7.88 (m, 2H) 6.60-6.67 (m, 1H) 5.91 (s, 2H) 5.08-5.46 (m, 1H) 3.99 (br d, J=11.56 Hz, 2H) 3.88 (br t, J=10.67 Hz, 2H) 3.72 (t, J=8.05 Hz, 2H) 3.61 (br d, J=11.32 Hz, 2H) 2.06-2.22 (m, 2H) 1.65-1.80 (m, 2H) 1.41-1.47 (m, 9H) 0.89 (t, J=8.05 Hz, 2H)-0.06 (s, 9H).

### Synthesis of Intermediate C158

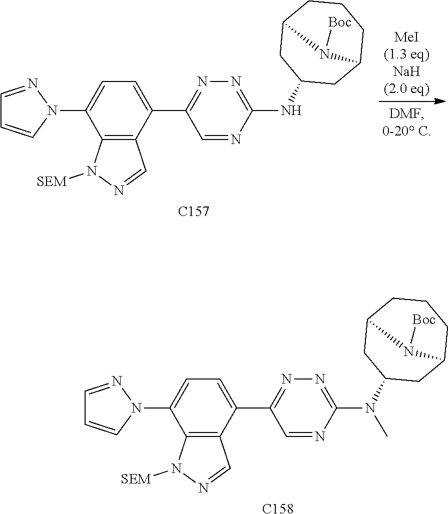 [SMILES: CC(C)(C)OC(=O)N1[C@@H]2CCC[C@H]1C[C@H](Nc1ncc(-c3ccc(-n4cccn4)c4c3cnn4COCC[Si](C)(C)C)nn1)C2 ; "C157" ; CN(c1ncc(-c2ccc(-n3cccn3)c3c2cnn3COCC[Si](C)(C)C)nn1)[C@@H]1C[C@H]2CCC[C@@H](C1)N2C(=O)OC(C)(C)C ; "C158"]

To a solution of tert-butyl (1R,5S)-3-[[6-[7-pyrazol-1-yl-1-(2-trimethylsilyl ethoxymethyl)indazol-4-yl]-1,2,4-triazin-3-yl]amino]-9-azabicyclo[3.3.1]nonane-9-carboxylate (C157, 95.0 mg, 150 μmol, 1 eq) in N,N-dimethyl formamide (0.500 mL) was added sodium hydride (60 wt %, 12.0 mg, 300 μmol, 2 eq) at 0° C. After completion of addition, the mixture was stirred at 20° C. for 30 min. Then iodomethane (28.0 mg, 195 μmol, 1.3 eq) was added dropwise at 0° C. The resulting mixture was stirred at 20° C. for 12 hrs. The reaction mixture was quenched with H₂O (20.0 mL) and the reaction mixture was extracted with dichloromethane (5×10 mL). The combined organic layer was dried over Na₂SO₄, filtered and the filtrate was concentrated under reduced pressure to give crude tert-butyl (1S,5R)-7-[methyl-[6-[7-pyrazol-1-yl-1-(2-trimethylsilylethoxymethyl)indazol-4-yl]-1,2,4-triazin-3-yl]amino]-3-oxa-9-azabicyclo [3.3.1]nonane-9-carboxylate (C158, 40.0 mg, 41%) as a solid. The crude product was used in the next step directly without further purification. LCMS: (ES, m/z): 646.3 [M+H]⁺.

### Synthesis of Compound 546

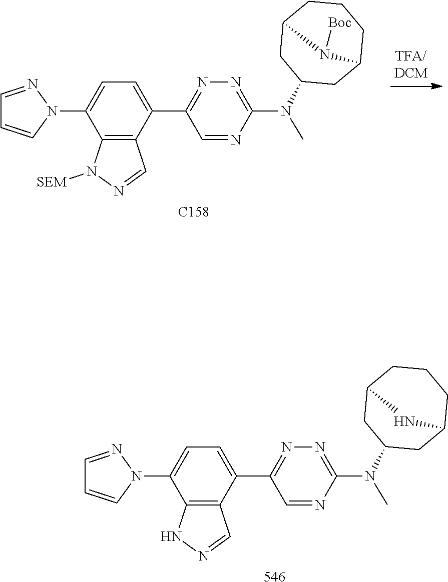 [SMILES: CN(c1ncc(-c2ccc(-n3cccn3)c3c2cnn3COCC[Si](C)(C)C)nn1)[C@@H]1C[C@H]2CCC[C@@H](C1)N2C(=O)OC(C)(C)C ; "C158" ; CN(c1ncc(-c2ccc(-n3cccn3)c3[nH]ncc23)nn1)[C@@H]1C[C@H]2CCC[C@@H](C1)N2 ; "546"]

To a solution of tert-butyl (1R,5S)-3-[methyl-[6-[7-pyrazol-1-yl-1-(2-trimethylsilyl ethoxymethyl)indazol-4-yl]-1,2,4-triazin-3-yl]amino]-9-azabicyclo[3.3.1]nonane-9-carboxylate (C158, 35.0 mg, 55.3 μmol, 1 eq) in dichloromethane (0.525 mL) was added dropwise trifluoroacetic acid (0.175 mL) at 0° C. The resulting mixture was stirred at 20° C. for 12 hrs. The reaction mixture was concentrated under reduced pressure to give a residue, which was purified by prep-HPLC (Conditio n14, Gradient 6) to give (1R,5S)-N-methyl-N-[6-(7-pyrazol-1-yl-1H-indazol-4-yl)-1,2,4-triazin-3-yl]-9-azabicyclo[3.3.1]nonan-3-amine (Compound 546, 22.1 mg, 98%) as a solid. LCMS: (ES, m/z): 416.1 [M+H]⁺. ¹HNMR (400 MHz, DMSO-d₆) δ ppm 9.11 (s, 1H) 8.76 (br s, 1H) 8.69 (s, 1H) 7.93 (s, 1H) 7.88 (d, J=7.75 Hz, 1H) 7.79 (d, J=8.11 Hz, 1H) 6.68 (s, 1H) 5.79-5.95 (m, 1H) 3.78 (br s, 2H) 3.13 (s, 3H) 2.34-2.41 (m, 2H) 1.91-2.06 (m, 7H) 1.74-1.82 (m, 1H).

### Example 140: Synthesis of Compound 484

### Synthesis of Intermediate C159

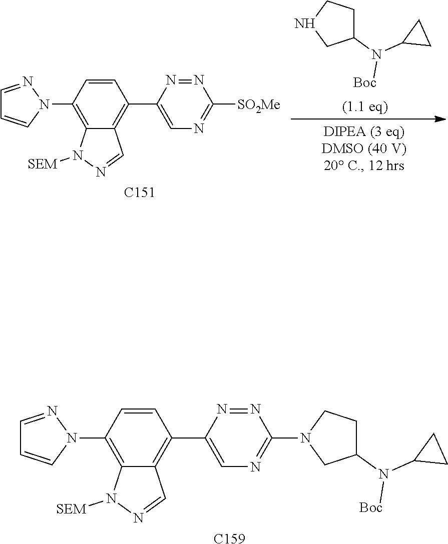 [SMILES: C[Si](C)(C)CCOCn1ncc2c(-c3cnc(S(C)(=O)=O)nn3)ccc(-n3cccn3)c21 ; CC(C)(C)OC(=O)N(C1CC1)C1CCNC1 ; "C151" ; CC(C)(C)OC(=O)N(C1CC1)C1CCN(c2ncc(-c3ccc(-n4cccn4)c4c3cnn4COCC[Si](C)(C)C)nn2)C1 ; "C159"]

A mixture of trimethyl-[2-[[4-(3-methylsulfonyl-1,2,4-triazin-6-yl)-7-pyrazol-1-yl-indazol-1-yl]methoxy]ethyl]silane (C151, 80.0 mg, 170 μmol, 1 eq), tert-butyl N-cyclopropyl-N-pyrrolidin-3-yl-carbamate (C5, 38.4 mg, 187 μmol, 1.1 eq), N,N-diisopropylethylamine (123 mg, 954 μmol, 3 eq) in dimethyl sulfoxide (3.2 mL) was stirred at 20° C. for 12 hrs. The reaction mixture was quenched with water (30 mL) at 20° C. and extracted with ethyl acetate (5×10 mL). The combined organic layer was dried over Na₂SO₄, filtered and the filtrate was concentrated under reduced pressure to give crude product, which was purified by column chromatography on silica gel (SiO₂, 29% of ethyl acetate in petroleum ether) to give tert-butyl N-cyclopropyl-N-[1-[6-[7-pyrazol-1-yl-1-(2-trimethylsilylethoxymet hyl) indazol-4-yl]-1,2,4-triazin-3-yl]pyrrolidin-3-yl]carbamate (C159, 40.0 mg, 38%) as a solid. LCMS: (ES, m/z): 618.2 [M+H]⁺. ¹HNMR (400 MHz, DMSO-d₆) δ ppm 9.23-9.30 (m, 2H) 9.12 (s, 1H) 8.00 (d, J=7.70 Hz, 1H) 7.80-7.92 (m, 2H) 6.61-6.66 (m, 1H) 5.91 (s, 2H) 4.35-4.47 (m, 1H) 3.86-4.06 (m, 2H) 3.72 (br t, J=8.01 Hz, 2H) 3.47-3.67 (m, 2H) 2.34-2.48 (m, 2H) 2.22-2.32 (m, 1H) 1.42 (s, 9H) 0.90 (t, J=8.01 Hz, 2H) 0.73-0.80 (m, 2H) 0.61-0.70 (m, 2H)-0.06 (s, 9H).

### Synthesis of Compound 484

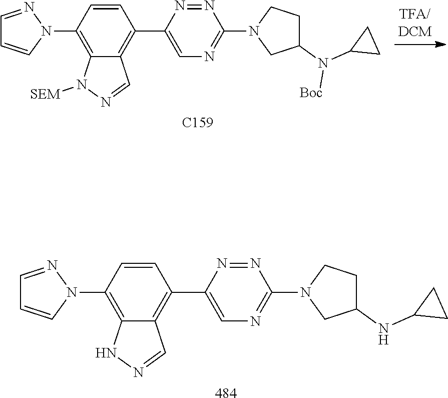 [SMILES: CC(C)(C)OC(=O)N(C1CC1)C1CCN(c2ncc(-c3ccc(-n4cccn4)c4c3cnn4COCC[Si](C)(C)C)nn2)C1 ; "C159" ; c1cnn(-c2ccc(-c3cnc(N4CCC(NC5CC5)C4)nn3)c3cn[nH]c23)c1 ; "484"]

To a solution of tert-butyl N-cyclopropyl-N-[1-[6-[7-pyrazol-1-yl-1-(2-trimethylsilyl ethoxymethyl)indazol-4-yl]-1,2,4-triazin-3-yl]pyrrolidin-3-yl]carbamate (C159, 40.0 mg, 64.7 μmol, 1 eq) in dichloromethane (0.600 mL) was added dropwise trifluoroacetic acid (0.2 mL) at 0° C. The resulting mixture was stirred at 20° C. for 12 hrs. The reaction mixture was concentrated under reduced pressure to give a residue, which was purified by prep-HPLC (condition 14, Gradient 1) to give N-cyclopropyl-1-[6-(7-pyrazol-1-yl-1H-indazol-4-yl)-1,2,4-triazin-3-yl]pyrrolidin-3-amine (Compound 484, 16.6 mg, 66%) as a solid. LCMS: (ES, m/z): 388.2 [M+H]⁺. ¹HNMR (400 MHz, METHANOL-d₄) δ ppm 8.93 (s, 1H) 8.74 (s, 1H) 8.51 (br s, 1H) 7.92 (s, 1H) 7.74-7.80 (m, 1H) 7.65-7.73 (m, 1H) 6.63 (s, 1H) 3.81-4.02 (m, 2H) 3.55-3.76 (m, 3H) 2.29-2.41 (m, 1H) 2.20-2.28 (m, 1H) 2.04 (br dd, J=11.56, 6.44 Hz, 1H) 0.55 (br d, J=6.79 Hz, 2H) 0.43 (br d, J=2.50 Hz, 2H).

### Example 141: Synthesis of Compound 484

### Synthesis of Intermediate C159

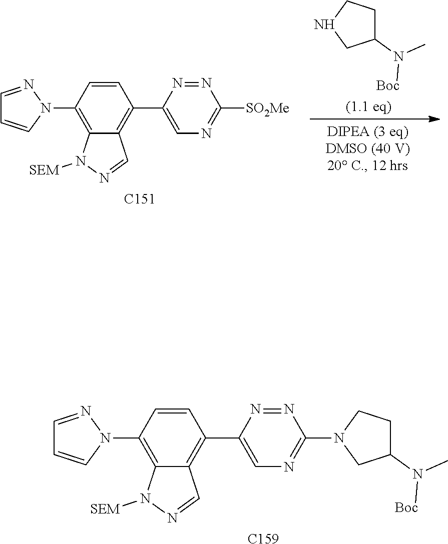 [SMILES: C[Si](C)(C)CCOCn1ncc2c(-c3cnc(S(C)(=O)=O)nn3)ccc(-n3cccn3)c21 ; CN(C(=O)OC(C)(C)C)C1CCNC1 ; "C151" ; CN(C(=O)OC(C)(C)C)C1CCN(c2ncc(-c3ccc(-n4cccn4)c4c3cnn4COCC[Si](C)(C)C)nn2)C1 ; "C159"]

A mixture of trimethyl-[2-[[4-(3-methylsulfonyl-1,2,4-triazin-6-yl)-7-pyrazol-1-yl-indazol-1-yl]methoxy]ethyl]silane (C151, 80.0 mg, 170 μmol, 1 eq), tert-butyl N-methyl-N-pyrrolidin-3-yl-carbamate (37.3 mg, 187 μmol, 1.1 eq), N,N-diisopropylethylamine (123 mg, 954 μmol, 3 eq) in dimethyl sulfoxide (3.2 mL) was stirred at 20° C. for 12 hrs. The reaction mixture was quenched with water (30 mL) at 20° C. and extracted with ethyl acetate (5×10 mL). The organic layer was dried over Na₂SO₄, filtered and the filtrate was concentrated under reduced pressure to give crude product, which was purified by column chromatography on silica gel (SiO₂, 29% of ethyl acetate in petroleum ether) to give tert-butyl N-methyl-N-[1-[6-[7-pyrazol-1-yl-1-(2-trimethylsilylethoxymethyl)indazol-4-yl]-1,2,4-triazin-3-yl] pyrrolidin-3-yl]carbamate (C159, 40.0 mg, 40%) as a solid. LCMS: (ES, m/z): 592.1 [M+H]⁺. ¹HNMR (400 MHz, DMSO-d₆) δ ppm 9.23-9.32 (m, 2H) 9.13 (s, 1H) 8.00 (d, J=7.87 Hz, 1H) 7.81-7.91 (m, 2H) 6.61-6.67 (m, 1H) 5.91 (s, 2H) 4.73-4.85 (m, 1H) 3.79-3.96 (m, 2H) 3.68-3.78 (m, 2H) 3.51-3.66 (m, 2H) 2.80 (s, 3H) 2.13-2.23 (m, 2H) 1.43 (s, 9H) 0.85-0.94 (m, 2H)-0.10-0.03 (m, 9H).

### Synthesis of Compound 485

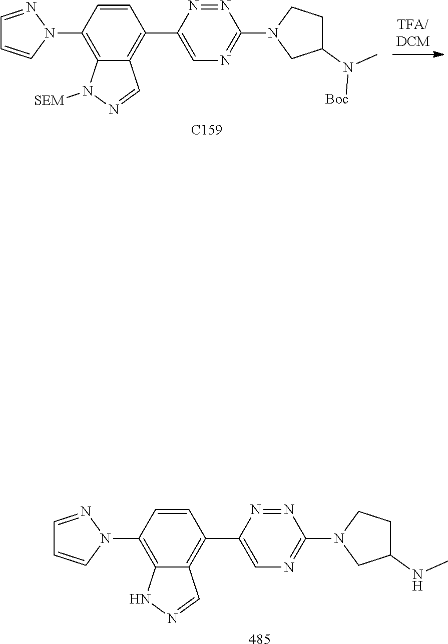 [SMILES: CN(C(=O)OC(C)(C)C)C1CCN(c2ncc(-c3ccc(-n4cccn4)c4c3cnn4COCC[Si](C)(C)C)nn2)C1 ; "C159" ; CNC1CCN(c2ncc(-c3ccc(-n4cccn4)c4[nH]ncc34)nn2)C1 ; "485"]

To a solution of tert-butyl N-methyl-N-[1-[6-[7-pyrazol-1-yl-1-(2-trimethylsilyl ethoxymethyl)indazol-4-yl]-1,2,4-triazin-3-yl]pyrrolidin-3-yl]carbamate (C159, 40.0 mg, 67.6 μmol, 1 eq) in dichloromethane (0.6 mL) was added dropwise trifluoroacetic acid (0.2 mL) at 0° C. The resulting mixture was stirred at 20° C. for 12 hrs. The reaction mixture was concentrated under reduced pressure to give a residue, which was purified by prep-HPLC (Condition 14, Gradient 1) to give N-methyl-1-[6-(7-pyrazol-1-yl-1H-indazol-4-yl)-1,2,4-triazin-3-yl]pyrrolidin-3-amine (Compound 485, 12.1 mg, 49.1%) as a solid. LCMS: (ES, m/z): 362.1 [M+H]⁺. ¹HNMR (400 MHz, METHANOL-d₄) δ ppm 8.72 (d, J=1.19 Hz, 1H) 8.56 (d, J=2.62 Hz, 1H) 7.95 (d, J=1.67 Hz, 1H) 7.79 (d, J=8.11 Hz, 1H) 7.67 (d, J=8.11 Hz, 1H) 6.64-6.69 (m, 1H) 5.98 (s, 1H) 4.06-4.18 (m, 2H) 3.79-4.01 (m, 3H) 2.85 (s, 3H) 2.64 (dtd, J=14.44, 8.43, 8.43, 6.26 Hz, 1H) 2.36-2.55 (m, 1H).

### Example 142: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (I)). After treatment, the levels of the HTT, MYB, or SMN2 mRNA targets were determined from each sample of cell lysate by cDNA synthesis followed by qPCR.

### Materials:

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_ml) —used for K562/suspension cell lines
GUSB: VIC-PL, ThermoFisher 4326320E (Assay: Hs99999908_ml) —used for K562/suspension cell lines
PPIA: VIC-PL, ThermoFisher 4326316E (Assay: Hs99999904_ml) —used for A673/adherent cell lines

### Probe/Primer Sequences

Canonical junction (CJ)
HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/
Alternative junction (AJ)
HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABkFQ/
MYB Primer 1:
CAACACCATTTCATAGAGACCAGAC
MYB Primer 2:
GTTCTAAAATCATCCCTTGGCTTCTAAT
MYB FAM-Probe:
/56-FAM/AAATACTGT/ZEN/ATAGGACCTCTTCTGACATCC/
3IABkFQ/

### Description

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO₂ for 24 hours.

The cells were then gently washed with 50 uL-100 uL cold PBS before proceeding to addition of lysis buffer. 30 uL-50 μL of room temperature lysis buffer with DNAse I (and optionally RNAsin) was added to each well. Cells were shaken/mixed thoroughly at room temperature for 5-10 minutes for lysis to take place and then 3 uL-5 μL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3× lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 μL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time | |
|---|---|---|---|---|
| RT step | 1 | 50° C. | 5 | min |
| RT inactivation/initial | 1 | 95° C. | 20 | sec |
| denaturation | | | | |
| Amplification | 40 | 95° C. | 3 | sec |
| | | 60° C. | 30 | sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 Å673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 50% response in AJ increase) while the decrease in CJ mRNA levels is reported as IC₅₀ (compound concentration having 50% response in CJ decrease).

A summary of these results is illustrated in Table 9, wherein “A” represents an AC₅₀ of less than 500 nM; “B” represents an AC₅₀ of between 500 nM and 5 μM; and “C” represents an AC₅₀ of greater than 5 μM.

TABLE 9
Modulation of RNA Splicing by Exemplary Compounds

| Compound | HTT CJ | HTT AJ | MYB CJ | MYB AJ |
|---|---|---|---|---|
| No. | IC₅₀ (nM) | AC₅₀ (nM) | IC₅₀ (nM) | AC₅₀ (nM) |
| 104 | B | C | C | C |
| 105 | A | B | A | B |
| 108 | — | — | D | D |
| 111 | | D | C | D |
| 112 | — | — | C | C |
| 113 | — | — | D | D |
| 115 | — | — | D | D |
| 117 | — | C | C | C |
| 118 | — | D | D | C |
| 119 | B | C | B | B |
| 121 | — | — | D | D |
| 131 | C | D | D | D |
| 132 | B | B | B | C |
| 137 | B | B | C | C |
| 138 | B | B | B | B |
| 142 | B | B | B | B |
| 143 | A | A | B | B |
| 146 | A | A | B | B |
| 149 | A | A | C | C |
| 150 | B | C | B | B |
| 170 | C | C | C | C |
| 174 | C | C | C | C |
| 175 | D | C | D | C |
| 176 | D | D | D | D |
| 177 | D | D | C | D |
| 178 | D | D | C | D |
| 179 | B | B | B | B |
| 180 | B | B | B | B |
| 181 | D | D | D | D |
| 182 | C | C | C | D |
| 183 | B | B | B | B |
| 184 | B | A | B | A |
| 185 | D | C | C | D |
| 186 | A | A | B | D |
| 187 | D | D | D | D |
| 188 | D | D | D | D |
| 189 | D | D | D | D |
| 190 | D | D | D | D |
| 191 | D | D | D | D |
| 192 | D | D | D | D |
| 193 | D | D | D | D |
| 196 | D | D | D | D |
| 197 | C | C | D | D |
| 198 | D | D | D | D |
| 200 | D | D | D | D |
| 201 | C | C | C | C |
| 202 | A | A | B | B |
| 203 | B | B | B | B |
| 204 | A | A | A | B |
| 205 | D | D | D | D |
| 206 | C | D | C | B |
| 210 | A | A | A | A |
| 211 | B | B | B | B |
| 213 | A | A | B | B |
| 214 | D | C | C | D |
| 215 | A | A | B | A |
| 216 | A | A | B | A |
| 218 | D | D | D | D |
| 219 | B | B | B | B |
| 220 | C | D | C | D |
| 221 | D | D | D | D |
| 222 | C | C | C | C |
| 223 | C | C | C | D |
| 224 | D | D | C | D |
| 225 | C | C | B | D |
| 226 | A | A | A | A |
| 227 | A | A | A | A |
| 228 | A | B | B | B |
| 229 | C | C | C | C |
| 230 | D | D | D | D |
| 231 | B | B | B | B |
| 232 | C | B | C | B |
| 233 | B | B | B | B |
| 234 | D | D | D | D |
| 235 | D | D | D | D |
| 237 | D | D | D | D |
| 238 | D | D | D | D |
| 239 | D | D | D | D |
| 240 | D | D | D | D |
| 241 | D | C | D | C |
| 242 | D | D | D | D |
| 243 | C | C | C | C |
| 251 | D | D | D | D |
| 252 | D | D | D | D |
| 253 | D | D | D | D |
| 254 | D | D | D | C |
| 255 | D | D | D | C |
| 263 | D | D | D | D |
| 264 | D | D | D | D |
| 265 | D | D | C | D |
| 266 | C | D | C | D |
| 267 | D | D | D | D |
| 273 | D | D | D | D |
| 274 | D | D | D | D |
| 275 | B | C | B | C |
| 276 | C | C | C | C |
| 277 | D | D | D | D |
| 278 | A | B | B | B |
| 279 | C | C | C | D |
| 280 | B | B | C | C |
| 281 | D | D | C | D |
| 283 | C | C | D | D |
| 284 | B | B | B | D |
| 285 | D | C | D | D |
| 287 | D | D | D | D |
| 288 | D | D | D | D |
| 289 | D | D | C | D |
| 290 | A | A | C | B |
| 291 | A | A | A | A |
| 292 | B | B | B | C |
| 293 | C | C | C | D |
| 294 | B | B | B | B |
| 295 | A | A | A | B |
| 296 | A | A | B | A |
| 297 | B | B | B | B |
| 300 | B | B | C | B |
| 301 | D | D | D | D |
| 302 | D | D | D | D |
| 303 | D | D | D | D |
| 304 | D | D | D | D |
| 305 | B | B | C | C |
| 306 | D | C | D | D |
| 307 | B | B | B | C |
| 308 | A | A | A | A |
| 310 | C | C | C | D |
| 311 | A | A | B | B |
| 312 | C | C | C | B |
| 313 | A | B | B | B |
| 314 | A | B | B | B |
| 315 | C | C | C | C |
| 316 | B | B | B | B |
| 317 | B | B | B | B |
| 318 | C | C | C | C |
| 319 | B | B | B | C |
| 320 | B | B | B | B |
| 321 | B | B | B | B |
| 322 | A | A | A | A |
| 323 | B | B | B | B |
| 324 | C | C | C | C |
| 325 | C | C | C | C |
| 326 | B | B | B | C |
| 327 | A | A | A | A |
| 328 | D | D | D | D |
| 329 | D | D | D | D |
| 330 | A | A | B | B |
| 331 | C | C | C | C |
| 334 | B | C | D | C |
| 335 | C | C | C | C |
| 336 | B | B | B | B |
| 337 | D | C | D | D |
| 338 | C | C | C | C |
| 339 | A | A | A | A |
| 342 | B | B | C | C |
| 343 | A | B | B | B |
| 344 | A | B | B | B |
| 345 | C | C | C | C |
| 346 | D | D | D | D |
| 347 | D | D | D | D |
| 348 | A | B | B | B |
| 349 | C | C | C | D |
| 350 | D | D | D | D |
| 351 | A | A | B | B |
| 353 | D | C | C | D |
| 354 | A | B | B | C |
| 355 | C | C | C | C |
| 356 | A | A | B | C |
| 357 | A | A | B | B |
| 358 | D | D | C | D |
| 359 | D | D | D | D |
| 360 | A | A | B | B |
| 362 | A | A | B | A |
| 363 | A | B | B | B |
| 364 | A | A | B | A |
| 365 | C | C | D | D |
| 366 | C | C | C | C |
| 370 | D | D | D | D |
| 373 | B | B | C | B |
| 374 | D | D | D | D |
| 375 | D | D | D | D |
| 376 | A | B | B | B |
| 378 | A | A | B | A |
| 379 | A | A | B | B |
| 380 | C | C | C | C |
| 409 | A | A | B | B |
| 410 | D | D | C | D |
| 411 | C | D | D | D |
| 412 | B | C | D | D |
| 413 | B | B | B | B |
| 414 | B | B | C | D |
| 415 | A | A | B | B |
| 416 | C | C | C | D |
| 417 | C | D | C | C |
| 418 | A | A | A | A |
| 419 | A | A | B | B |
| 420 | A | A | B | B |
| 421 | A | A | B | B |
| 422 | A | B | B | C |
| 424 | A | A | B | B |
| 425 | B | B | C | D |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for four exemplary genes (HTT, SMN2, MYB, and Target C) analyzed in this panel are reported as IC₅₀ (compound concentration having 50% response in CJ decrease).

A summary of the results from the panel is illustrated in Table 10, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater

than 10 μM.

TABLE 10
Modulation of RNA Splicing by Exemplary Compounds

| Compound | | | | Target |
|---|---|---|---|---|
| No. | HTT | MYB | SMN2 | C |
| 104 | B | C | A | B |
| 105 | A | A | A | A |
| 108 | | D | | |
| 111 | | C | | D |
| 112 | | C | | |
| 113 | | D | | |
| 115 | | D | | |
| 117 | | C | | C |
| 118 | | D | | C |
| 119 | B | B | B | B |
| 121 | | D | | |
| 131 | C | D | B | |
| 132 | B | B | A | B |
| 137 | B | C | B | C |
| 138 | B | B | B | B |
| 142 | B | B | A | B |
| 143 | A | B | A | B |
| 146 | A | B | A | A |
| 149 | A | C | B | B |
| 150 | B | B | B | |
| 170 | C | C | B | D |
| 174 | C | C | B | D |
| 175 | D | D | C | D |
| 176 | D | D | D | D |
| 177 | D | C | C | D |
| 178 | D | C | D | D |
| 179 | B | B | B | B |
| 180 | B | B | B | B |
| 181 | D | D | A | D |
| 182 | C | C | B | C |
| 183 | B | B | B | B |
| 184 | B | B | A | B |
| 185 | D | C | B | D |
| 186 | A | B | A | B |
| 187 | D | D | C | D |
| 188 | D | D | D | D |
| 189 | D | D | D | D |
| 190 | D | D | D | D |
| 191 | D | D | C | D |
| 192 | D | D | D | D |
| 193 | D | D | D | D |
| 196 | D | D | D | D |
| 197 | C | D | C | D |
| 198 | D | D | B | D |
| 200 | D | D | D | D |
| 201 | C | C | B | C |
| 202 | A | B | A | B |
| 203 | B | B | A | B |
| 204 | A | A | A | A |
| 205 | D | D | C | D |
| 206 | C | C | A | D |
| 210 | A | A | A | A |
| 211 | B | B | B | B |
| 213 | A | B | A | A |
| 214 | D | C | B | D |
| 215 | A | B | A | A |
| 216 | A | B | A | B |
| 218 | D | D | D | D |
| 219 | B | B | A | B |
| 220 | C | C | B | D |
| 221 | D | D | D | D |
| 222 | C | C | B | C |
| 223 | C | C | B | C |
| 224 | D | C | C | D |
| 225 | C | B | B | C |
| 226 | A | A | A | A |
| 227 | A | A | A | A |
| 228 | A | B | A | A |
| 229 | C | C | C | C |
| 230 | D | D | C | D |
| 231 | B | B | A | B |
| 232 | C | C | B | C |
| 233 | B | B | B | B |
| 234 | D | D | B | D |
| 235 | D | D | C | D |
| 237 | D | D | D | D |
| 238 | D | D | D | D |
| 239 | D | D | D | D |
| 240 | D | D | D | D |
| 241 | D | D | C | D |
| 242 | D | D | C | D |
| 243 | C | C | B | D |
| 251 | D | D | D | D |
| 252 | D | D | C | D |
| 253 | D | D | D | D |
| 254 | D | D | B | D |
| 255 | D | D | B | D |
| 263 | D | D | D | D |
| 264 | D | D | C | D |
| 265 | D | C | B | D |
| 266 | C | C | D | D |
| 267 | D | D | C | D |
| 273 | D | D | C | D |
| 274 | D | D | C | D |
| 275 | B | B | B | C |
| 276 | C | C | C | C |
| 277 | D | D | D | D |
| 278 | A | B | A | B |
| 279 | C | C | B | D |
| 280 | B | C | B | C |
| 281 | D | C | B | D |
| 283 | C | D | C | C |
| 284 | B | B | B | C |
| 285 | D | D | B | D |
| 287 | D | D | B | D |
| 288 | D | D | C | D |
| 289 | D | C | D | D |
| 290 | A | C | B | B |
| 291 | A | A | A | A |
| 292 | B | B | A | C |
| 293 | C | C | B | D |
| 294 | B | B | A | B |
| 295 | A | A | A | A |
| 296 | A | B | A | A |
| 297 | B | B | A | B |
| 300 | B | C | B | B |
| 301 | D | D | D | D |
| 302 | D | D | D | D |
| 303 | D | D | C | D |
| 304 | D | D | C | D |
| 305 | B | C | A | C |
| 306 | D | D | B | D |
| 307 | B | B | A | B |
| 308 | A | A | A | A |
| 310 | C | C | B | D |
| 311 | A | B | A | B |
| 312 | C | C | B | C |
| 313 | A | B | A | B |
| 314 | A | B | A | B |
| 315 | C | C | B | D |
| 316 | B | B | A | C |
| 317 | B | B | C | C |
| 318 | C | C | C | C |
| 319 | B | B | A | C |
| 320 | B | B | A | C |
| 321 | B | B | A | B |
| 322 | A | A | A | A |
| 323 | B | B | A | C |
| 324 | C | C | B | D |
| 325 | C | C | B | D |
| 326 | B | B | A | C |
| 327 | A | A | A | A |
| 328 | D | D | C | D |
| 329 | D | D | C | D |
| 330 | A | B | A | A |
| 331 | C | C | B | D |
| 334 | B | D | B | D |
| 335 | C | C | B | C |
| 336 | B | B | A | D |
| 337 | D | D | B | D |
| 338 | C | C | B | D |
| 339 | A | A | A | A |
| 342 | B | C | A | C |
| 343 | A | B | A | B |
| 344 | A | B | A | B |
| 345 | C | C | B | C |
| 346 | D | D | D | D |
| 347 | D | D | C | D |
| 348 | A | B | A | B |
| 349 | C | C | A | C |
| 350 | D | D | D | D |
| 351 | A | B | A | B |
| 353 | D | C | C | C |
| 354 | A | B | A | B |
| 355 | C | C | C | C |
| 356 | A | B | A | A |
| 357 | A | B | A | B |
| 358 | D | C | D | D |
| 359 | D | D | D | D |
| 360 | A | B | B | B |
| 362 | A | B | A | B |
| 363 | A | B | A | B |
| 364 | A | B | A | B |
| 365 | C | D | C | C |
| 366 | C | C | B | C |
| 370 | D | D | C | C |
| 373 | B | C | B | C |
| 374 | D | D | D | D |
| 375 | D | D | D | D |
| 376 | A | B | A | B |
| 378 | A | B | A | B |
| 379 | A | B | A | B |
| 380 | C | C | B | C |
| 409 | A | B | A | A |
| 410 | D | C | C | D |
| 411 | C | D | C | D |
| 412 | B | D | B | D |
| 413 | B | B | B | B |
| 414 | B | C | C | C |
| 415 | A | B | A | B |
| 416 | C | C | C | D |
| 417 | C | C | B | D |
| 418 | A | A | A | A |
| 419 | A | B | A | B |
| 420 | A | B | B | B |
| 421 | A | B | A | A |
| 422 | A | B | B | B |
| 424 | A | B | B | A |
| 425 | B | C | C | B |
| 443 | A | C | B | B |
| 449 | A | B | A | A |
| 451 | C | C | C | C |
| 484 | C | D | C | D |
| 485 | C | C | C | D |
| 511 | A | C | B | B |
| 548 | C | D | C | D |
| 549 | C | C | C | D |
| 551 | A | B | A | A |
| 552 | B | C | B | C |
| 553 | C | C | C | C |
| 554 | B | B | B | C |
| 555 | A | C | B | B |
| 556 | A | B | B | B |
| 558 | B | B | A | B |
| 559 | B | C | B | C |
| 560 | B | C | B | C |
| 561 | A | B | B | B |
| 562 | D | D | C | D |
| 563 | C | C | C | D |
| 564 | B | C | B | C |
| 565 | C | C | C | D |
| 566 | B | C | C | C |
| 567 | B | C | B | B |
| 568 | B | D | C | C |
| 569 | B | C | C | C |
| 570 | B | B | B | B |
| 571 | C | C | C | C |
| 572 | A | B | B | B |
| 573 | C | D | D | D |
| 574 | A | B | A | A |
| 575 | B | D | C | C |
| 576 | A | C | B | B |
| 577 | B | D | C | D |
| 578 | A | B | A | B |
| 579 | B | C | B | B |
| 580 | A | B | A | A |
| 581 | A | B | B | B |
| 582 | B | C | A | C |
| 585 | D | D | D | D |

TABLE 10a
Modulation of RNA Splicing by
Exemplary Compounds

| Compound | | | | |
|---|---|---|---|---|
| No. | HTT | MYB | SMN2 | Target C |
| 901 | | D | | |
| 903 | | D | | D |
| 906 | A | B | A | A |
| 907 | B | C | B | D |
| 908 | D | D | C | C |
| 909 | C | C | B | |
| 912 | C | C | B | C |
| 913 | C | C | C | C |
| 914 | B | C | A | B |
| 915 | B | B | D | C |
| 916 | D | D | D | D |
| 917 | B | B | A | B |
| 918 | B | B | A | B |
| 919 | A | B | A | B |
| 920 | B | B | A | B |
| 921 | D | D | C | D |
| 922 | A | B | A | B |
| 923 | A | B | A | B |
| 924 | C | D | B | D |
| 925 | C | C | B | D |
| 926 | B | C | B | C |
| 927 | B | C | B | C |
| 928 | B | B | A | B |
| 929 | A | A | A | A |
| 930 | A | B | A | B |
| 931 | A | A | A | A |
| 932 | B | B | B | B |
| 933 | D | D | C | D |
| 934 | C | D | C | D |
| 935 | B | B | B | B |
| 936 | B | C | A | B |
| 937 | B | B | A | C |
| 938 | C | D | C | C |
| 939 | A | B | A | B |
| 940 | A | B | A | A |
| 941 | D | D | C | D |
| 942 | C | D | A | C |
| 943 | C | D | D | D |
| 944 | B | C | B | C |
| 945 | B | C | A | C |
| 946 | C | C | B | D |
| 948 | B | C | B | C |
| 949 | C | D | C | D |
| 950 | B | B | A | C |
| 951 | B | B | B | B |
| 954 | C | C | B | C |
| 955 | A | B | A | B |
| 956 | A | B | A | B |
| 957 | C | C | C | C |
| 958 | D | D | C | D |
| 959 | A | B | A | B |
| 960 | C | C | B | C |
| 961 | B | B | B | B |
| 962 | B | B | B | C |
| 963 | C | C | C | D |
| 964 | D | D | D | D |
| 965 | A | B | A | B |
| 966 | A | B | A | A |
| 967 | A | B | A | B |
| 968 | B | C | B | C |
| 969 | C | D | C | D |
| 970 | D | D | C | D |
| 971 | C | C | C | C |
| 972 | B | C | B | C |
| 978 | D | D | D | D |
| 979 | A | B | A | B |
| 980 | C | C | D | C |
| 981 | B | C | B | C |
| 982 | A | B | A | B |
| 983 | C | C | B | C |
| 984 | B | C | B | C |
| 986 | C | C | B | C |
| 987 | D | D | C | D |
| 988 | C | D | B | D |
| 989 | B | C | B | C |
| 990 | D | D | C | D |
| 991 | D | D | D | D |
| 992 | C | D | C | D |
| 993 | A | B | A | B |
| 994 | B | B | C | B |
| 995 | C | D | C | D |
| 996 | B | C | B | C |
| 997 | D | D | D | D |
| 998 | B | D | B | D |
| 999 | C | C | B | C |

### Example 143: Exemplary In Vitro Assays for Measuring Membrane Efflux and Permeability

Compounds described herein were screened for cell membrane permeability and efflux in a transwell assay using Madin-Darby Canine Kidney (MDCK) cells expressing Breast Cancer Resistance Protein (BCRP) or subclone MDCKII cells expressing Multidrug Resistance Protein 1 (MDR1).

### Materials:

MDCK-BCRP or MDCKII-MDR1 Cells: The Netherlands Cancer Institute, High Glucose Dulbecco's Modified Eagle's Medium (DMEM): Gibco/Thermo Fisher Scientific, Fetal Bovine Serum (FBS): Coming, Hank's Balanced Salt Solution (HBSS) and Trypsin/EDTA: Gibco/Thermo Fisher Scientific, HTS Transwell-96 Well Permeable Supports: Coming Corporation, Millicell Epithelial Volt-Ohm: Millipore, Cellometer® Vision: Nexcelom Bioscience LLC, Infinite 200 PRO microplate reader: Tecan, MTS2/4 orbital shaker: IKA Labortechnik, HEPES/Penicillin/Streptomycin: Solarbio.

### DESCRIPTION

MDCK-BCRP or MDCKII-MDR1 cells were seeded to each of the wells of the transwell plate at a density of 1.6×10⁶ cells/mL and the plate was incubated for 4-8 days with medium changes every two days. To perform the drug transport assay, cell monolayers were first washed three times with pre-warmed HBSS and then incubated with gentle shaking (150 rpm) for 30 min at 37° C. Stock solutions of test compounds (e.g., exemplary compounds of Formulas (I), (II), and (III)) and control compounds (e.g., metoprolol, prazosin, and/or imatinib) were prepared in DMSO and adjusted to a working solution using HBSS. The final concentration of test compounds was 1 μM with DMSO; 0.5%. The plates were incubated at 37° C. for 2 hours. Permeability and efflux were measured according to published protocols (e.g., those described in Drug Metabolism and Disposition 36, 268-275 (2008) and Journal of Pharmaceutical Sciences 107 2225-2235 (2018)).

A summary of the results for permeability and efflux ratio are provided in Table 11, wherein for efflux ratio, A represents a ratio less than 1.5; B represents a ratio between 1.5 and 5; and C represents a ratio greater than 5. For permeability, A represents a Papp <2×10⁻⁶ cm s⁻¹; B represents a Papp between 2−6×10⁻⁶ cm s⁻¹; and C represents a Papp greater than 6×10⁻⁶ cm S⁻¹.

TABLE 11
Cell permeability and efflux ratio for exemplary compounds

| | Efflux | |
|---|---|---|
| Cmpd No. | Ratio | Permeability |
| 104 | C | A |
| 105 | C | A |
| 132 | C | A |
| 137 | C | A |
| 147 | C | A |
| 178 | C | A |
| 182 | C | A |
| 183 | C | A |
| 184 | C | A |
| 186 | C | A |
| 202 | C | A |
| 203 | C | A |
| 204 | C | A |
| 210 | C | A |
| 213 | C | A |
| 215 | C | A |
| 216 | C | A |
| 226 | C | A |
| 227 | C | A |
| 228 | C | A |
| 242 | C | A |
| 291 | C | A |
| 294 | B | A |
| 307 | C | B |
| 311 | C | A |
| 313 | A | C |
| 327 | C | B |
| 330 | C | B |
| 336 | C | B |
| 337 | A | B |
| 339 | B | A |
| 348 | C | A |
| 356 | C | A |
| 362 | C | B |
| 363 | B | A |
| 378 | C | B |
| 379 | B | B |
| 385 | C | A |
| 386 | C | A |
| 415 | B | B |
| 418 | C | A |
| 551 | C | B |
| 556 | C | B |
| 561 | C | B |
| 572 | A | A |
| 574 | C | A |
| 576 | C | A |
| 578 | C | A |
| 579 | C | A |
| 580 | C | A |
| 581 | A | B |

TABLE 11a
Cell permeability and efflux
ratio for exemplary compounds

| Compound | Efflux | |
|---|---|---|
| No. | Ratio | Permeability |
| 906 | C | A |
| 915 | A | A |
| 917 | C | B |
| 918 | C | B |
| 919 | C | A |
| 920 | C | B |
| 922 | C | A |
| 930 | C | A |
| 931 | C | A |
| 939 | C | A |
| 940 | C | A |
| 954 | C | A |
| 955 | C | A |
| 956 | C | B |
| 961 | B | A |
| 962 | B | B |
| 965 | C | B |
| 966 | C | B |
| 967 | B | B |
| 969 | C | B |
| 982 | C | A |

### Example 144: Evaluating Effect of Exemplary Compounds on Protein Abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I), (II), or (III)). After treatment for 24 hours, the protein abundance of a specific target was determined by measuring luminescence.

### Materials:

Promega Nano-Glo HiBiT Lytic Detection System (cat #N3030)
Coming 384-well TC-treated microplates (cat #3570)
Synthego Engineered Cells Knock-In Clones

TABLE 12
Design of genetically modified HiBiT cell lines

| | | | Guide | Guide | |
|---|---|---|---|---|---|
| Cell | | | RNA | RNA cut | |
| Line | Gene | Modification | Sequence | location | Donor Sequence |
| K562 | MYB | HiBiT | GCGCCA | chr6: | CGGTGCGGTCCCCGCGGCTC |
| | | | TGGCCC | 135,181,526 | TCGGCGGAGCCCCGCGCCCG |
| | | | GAAGAC | | CCGCGCCATGgtgagcggctggcgg |
| | | | CC | | ctgttcaagaagattagcGGCAGCTCC |
| | | | | | GGAGGATCTAGCGGCGCCCG |
| | | | | | AAGACCCCGGCACAGgtaacgg |
| | | | | | ggagccgggcgggcggccgaggg |
| K562 | HTT | HiBiT | CAGCTTT | chr4: | CGAGTCGGCCCGAGGCCTCC |
| | | | TCCAGG | 3,074,830 | GGGGACTGCCGTGCCGGGCG |
| | | | GTCGCC | | GGAGACCGCCATGgtgagcggctg |
| | | | A | | gcggctgttcaagaagattagcGGCAGC |
| | | | | | TCCGGAGGATCTAGCGGCGC |
| | | | | | GACCCTGGAAAAGCTGATGA |
| | | | | | AGGCCTTCGAGTCCCTCAAG |
| | | | | | TCCTTCCA |

### DESCRIPTION

Cells were maintained in IMIDM with 10% FBS. Before the assay, cells were diluted with phenolphthalein-free growth media (IMIDM+100 FBS media) and were seeded in a 384-well plate at a density of 10000 cells/well (for each cell line listed in Table 12). Each compound was prepared as a 10-point 3-fold serial dilution in DMSO with the top dose at a final concentration of 10 μM in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25%. Treated cell plates were placed in an incubator at 37° C. with 5% CO₂ for 24 hours. After 24 hours, 25 μL of Complete HiBit Lytic reagent was added to each well at room temperature (e.g. one plate requiring 10 mL Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 12, the percent response for each respective cell line was calculated at each compound concentration as follows:

$$\% response = 100 * ( S - PC ) / ( NC - P C )$$

For the normalized response at each concentration, a four-parameter logistical regression was fit to the data and the response was interpolated at the 50% value to determine a concentration for protein abundance at 50% (IC₅₀) the untreated control.

A summary of the results for protein abundance is illustrated in Tables 13A and 13B, wherein A represents <100 nM; B represents 100-1000 nM; C represents 1000-9999 nM; and D represents greater than 10 μM.

TABLE 13A
Protein abundance data for exemplary compounds

| Compound No. | HTT | MYB | Target C |
|---|---|---|---|
| 101 | C | C | C |
| 103 | C | C | C |
| 104 | A | B | B |
| 105 | A | A | A |
| 107 | C | C | C |
| 108 | D | D | D |
| 110 | C | C | C |
| 111 | C | C | C |
| 112 | C | C | C |
| 113 | C | C | C |
| 115 | D | D | D |
| 117 | C | C | C |
| 118 | C | C | C |
| 121 | D | D | D |
| 123 | D | D | D |
| 132 | B | B | B |
| 137 | B | B | B |
| 142 | A | A | A |
| 143 | A | A | A |
| 146 | A | A | A |
| 147 | A | A | A |
| 151 | B | B | B |
| 152 | A | B | A |
| 163 | D | D | D |
| 165 | C | C | C |
| 170 | C | C | D |
| 174 | B | C | D |
| 175 | C | C | C |
| 176 | C | C | C |
| 177 | C | C | C |
| 178 | D | C | D |
| 179 | B | B | B |
| 180 | B | B | B |
| 181 | A | A | A |
| 182 | A | B | B |
| 183 | A | B | A |
| 184 | A | B | B |
| 185 | C | C | C |
| 186 | A | B | A |
| 187 | C | C | D |
| 188 | D | D | D |
| 189 | D | D | D |
| 190 | D | D | D |
| 191 | C | C | D |
| 192 | D | D | D |
| 193 | D | C | D |
| 196 | D | D | D |
| 197 | C | C | C |
| 198 | D | C | D |
| 200 | C | C | C |
| 201 | C | C | D |
| 202 | A | B | B |
| 203 | A | A | A |
| 204 | A | A | A |
| 205 | D | C | C |
| 206 | C | C | D |
| 209 | A | B | A |
| 210 | A | A | A |
| 211 | B | B | B |
| 213 | A | A | A |
| 214 | C | C | D |
| 215 | A | A | A |
| 216 | A | A | B |
| 218 | D | D | D |
| 219 | A | B | B |
| 220 | C | C | D |
| 221 | C | C | C |
| 222 | B | C | C |
| 223 | C | C | C |
| 224 | C | B | C |
| 225 | B | B | C |
| 226 | A | A | A |
| 227 | A | A | A |
| 228 | A | A | A |
| 229 | C | C | C |
| 230 | C | C | D |
| 231 | A | B | B |
| 232 | B | C | C |
| 233 | B | B | B |
| 234 | C | C | D |
| 235 | C | C | B |
| 237 | D | D | D |
| 238 | D | D | D |
| 239 | D | D | D |
| 240 | D | D | D |
| 241 | C | C | C |
| 242 | C | C | D |
| 243 | C | C | D |
| 251 | D | D | D |
| 252 | D | C | D |
| 253 | D | D | D |
| 254 | D | D | D |
| 255 | C | C | D |
| 263 | D | D | D |
| 264 | C | C | C |
| 265 | D | D | D |
| 266 | C | C | D |
| 267 | C | C | C |
| 273 | D | C | C |
| 274 | C | C | C |
| 275 | B | B | A |
| 276 | C | C | C |
| 277 | D | C | C |
| 278 | A | B | A |
| 279 | C | C | C |
| 280 | A | B | B |
| 281 | C | C | C |
| 282 | C | C | D |
| 283 | C | B | C |
| 284 | B | B | C |
| 285 | C | C | D |
| 287 | C | B | C |
| 288 | C | B | C |
| 289 | C | B | D |
| 291 | A | A | A |
| 292 | B | B | B |
| 293 | C | C | C |
| 294 | B | B | B |
| 295 | A | A | A |
| 296 | A | A | A |
| 297 | B | B | B |
| 299 | A | A | A |
| 300 | B | B | B |
| 301 | D | C | D |
| 302 | D | C | D |
| 303 | C | C | C |
| 304 | C | C | D |
| 305 | B | B | C |
| 306 | C | C | D |
| 307 | A | B | B |
| 310 | C | C | C |
| 311 | B | B | A |
| 312 | B | B | C |
| 313 | B | B | B |
| 314 | A | B | A |
| 315 | B | B | D |
| 316 | B | B | B |
| 317 | B | B | B |
| 318 | C | C | C |
| 319 | B | B | C |
| 320 | B | B | B |
| 321 | B | B | B |
| 322 | A | A | A |
| 323 | B | B | C |
| 324 | C | C | C |
| 325 | C | C | D |
| 326 | B | B | C |
| 327 | A | A | A |
| 328 | D | C | D |
| 329 | D | D | D |
| 330 | A | B | A |
| 331 | B | C | D |
| 334 | B | B | C |
| 335 | B | C | C |
| 336 | A | B | B |
| 337 | C | B | D |
| 338 | C | C | C |
| 339 | A | A | A |
| 342 | B | B | B |
| 343 | A | B | B |
| 344 | A | B | B |
| 345 | B | C | C |
| 346 | C | C | D |
| 347 | C | C | D |
| 348 | A | A | B |
| 349 | B | B | C |
| 350 | D | C | D |
| 351 | A | B | B |
| 353 | C | C | D |
| 354 | B | B | B |
| 355 | C | C | C |
| 356 | A | A | A |
| 357 | A | B | B |
| 358 | D | C | D |
| 359 | D | C | D |
| 360 | B | B | A |
| 362 | A | A | A |
| 363 | A | B | B |
| 364 | A | A | A |
| 365 | C | C | C |
| 366 | C | C | C |
| 370 | C | C | D |
| 373 | B | C | C |
| 374 | D | D | D |
| 375 | D | D | D |
| 376 | B | B | B |
| 378 | A | B | A |
| 379 | A | B | B |
| 380 | C | C | C |
| 383 | C | C | C |
| 384 | C | C | C |
| 385 | A | A | A |
| 386 | A | A | A |
| 389 | B | B | A |
| 410 | C | C | C |
| 411 | D | D | D |
| 412 | C | C | C |
| 413 | B | B | B |
| 414 | B | C | B |
| 415 | A | B | A |
| 416 | C | C | C |
| 417 | B | D | C |
| 418 | A | A | A |
| 419 | A | A | A |
| 420 | A | B | A |
| 421 | A | B | A |
| 422 | B | B | B |
| 424 | A | B | A |
| 425 | B | C | B |
| 443 | A | B | A |
| 449 | A | B | A |
| 451 | C | C | C |
| 484 | C | D | C |
| 485 | C | C | C |
| 511 | B | C | B |
| 548 | C | D | C |
| 549 | C | C | C |
| 551 | A | A | A |
| 552 | B | C | B |
| 553 | C | C | C |
| 554 | B | B | B |
| 555 | B | C | B |
| 556 | A | B | A |
| 558 | A | B | A |
| 559 | B | B | B |
| 560 | B | C | B |
| 561 | A | B | A |
| 562 | C | C | C |
| 563 | C | C | C |
| 564 | B | C | B |
| 565 | C | C | C |
| 566 | B | C | C |
| 567 | B | C | B |
| 568 | B | C | C |
| 569 | B | C | C |
| 570 | B | C | B |
| 571 | B | C | C |
| 572 | A | B | B |
| 573 | C | C | C |
| 574 | A | B | A |
| 575 | B | C | C |
| 576 | A | B | B |
| 577 | C | C | D |
| 578 | A | B | A |
| 579 | B | C | B |
| 580 | A | B | A |
| 581 | A | B | A |
| 582 | B | C | C |

TABLE 13B
Protein abundance data for exemplary compounds

| Compound No. | MYB | Target C | HTT |
|---|---|---|---|
| 900 | | | |
| 901 | D | D | D |
| 903 | D | D | D |
| 904 | D | D | D |
| 905 | C | B | B |
| 906 | A | A | A |
| 907 | C | C | B |
| 908 | C | D | C |
| 909 | C | C | C |
| 912 | C | C | C |
| 913 | C | C | C |
| 914 | C | B | C |
| 915 | C | C | B |
| 916 | D | D | D |
| 917 | B | A | A |
| 918 | B | B | A |
| 919 | B | A | A |
| 920 | B | B | A |
| 921 | C | D | D |
| 922 | A | A | A |
| 923 | B | A | A |
| 924 | C | D | D |
| 925 | C | C | C |
| 926 | C | B | B |
| 927 | B | B | B |
| 928 | B | B | B |
| 929 | A | A | A |
| 930 | A | A | A |
| 931 | A | A | A |
| 932 | B | B | B |
| 933 | C | C | C |
| 934 | C | C | C |
| 935 | B | B | B |
| 936 | B | B | B |
| 937 | B | C | B |
| 938 | C | C | C |
| 939 | A | A | A |
| 940 | A | A | A |
| 941 | D | D | D |
| 942 | B | B | A |
| 943 | C | D | C |
| 944 | B | C | C |
| 945 | B | B | B |
| 946 | C | D | C |
| 948 | B | B | B |
| 949 | C | C | C |
| 950 | B | B | B |
| 951 | B | B | A |
| 954 | C | C | B |
| 955 | A | A | A |
| 956 | B | B | A |
| 957 | C | C | C |
| 958 | D | D | D |
| 959 | B | B | A |
| 960 | C | D | C |
| 961 | C | B | B |
| 962 | B | B | B |
| 963 | C | D | C |
| 964 | D | D | D |
| 965 | B | A | A |
| 966 | A | A | A |
| 967 | B | A | A |
| 968 | C | C | B |
| 969 | B | B | B |
| 970 | D | D | D |
| 971 | C | C | C |
| 972 | C | C | B |
| 978 | C | D | C |
| 979 | B | A | A |
| 980 | C | C | C |
| 981 | B | B | A |
| 982 | B | A | A |
| 983 | C | C | B |
| 984 | B | B | B |
| 986 | C | C | B |
| 987 | C | C | D |
| 988 | C | C | C |
| 989 | C | B | B |
| 991 | D | C | C |
| 992 | C | C | B |
| 993 | B | B | A |
| 994 | C | B | B |
| 995 | C | C | C |
| 996 | C | B | B |
| 997 | D | D | D |
| 998 | D | C | B |
| 999 | C | D | C |

### Example 155: Investigating Effect of Exemplary Compounds on Cell Viability

Compounds described herein were screened for toxicity in K562 (human chronic myelogenous leukemia) and SH-SY5Y (human neuroblastoma) cells using a Cell Titer Glo 2.0 assay.

### Materials:

Promega CellTiter-Gbo® 2.0 Cell Viability Assay (cat #G9241)
Coming 384-well TC-treated microplates (cat #3570)

### Description:

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I), (II), or (III)) were first serially diluted in DMSO then diluted 1:100 with INMDM+10% FBS. The final concentration of DMSO was 0.1 Cm in each well. The cells were incubated for 72 hours at 37° C. and 5% CO₂ before assaying with Cell Titer Glo 2.0 reagent.

A summary of the results for viability is illustrated in Tables 14A and 14B, wherein A represents <100 nM; B represents 100-1000 nM; C represents 1000-9999 nM; and D represents greater than 10 μM in K562 cells.

TABLE 14A
Cell viability data for
exemplary compounds

| Cmpnd | |
|---|---|
| No. | Viability |
| 104 | B |
| 105 | A |
| 119 | B |
| 132 | B |
| 137 | C |
| 142 | B |
| 143 | B |
| 146 | A |
| 147 | A |
| 151 | C |
| 152 | C |
| 163 | D |
| 164 | B |
| 165 | C |
| 170 | C |
| 174 | D |
| 175 | D |
| 176 | C |
| 177 | D |
| 178 | D |
| 179 | B |
| 180 | B |
| 181 | B |
| 182 | B |
| 183 | B |
| 184 | B |
| 185 | C |
| 186 | B |
| 187 | B |
| 188 | D |
| 189 | D |
| 190 | C |
| 191 | B |
| 192 | C |
| 193 | C |
| 196 | C |
| 197 | C |
| 198 | C |
| 200 | B |
| 201 | C |
| 202 | B |
| 203 | B |
| 204 | A |
| 205 | C |
| 206 | C |
| 209 | B |
| 210 | A |
| 211 | B |
| 213 | A |
| 214 | C |
| 215 | B |
| 216 | B |
| 218 | C |
| 219 | B |
| 220 | C |
| 221 | C |
| 222 | B |
| 223 | B |
| 224 | B |
| 225 | B |
| 226 | A |
| 227 | A |
| 228 | B |
| 229 | C |
| 230 | C |
| 231 | B |
| 232 | C |
| 233 | B |
| 234 | D |
| 235 | B |
| 237 | D |
| 238 | C |
| 239 | D |
| 240 | C |
| 241 | C |
| 242 | C |
| 243 | C |
| 251 | C |
| 252 | B |
| 253 | C |
| 254 | C |
| 255 | B |
| 263 | D |
| 264 | C |
| 265 | C |
| 266 | C |
| 267 | C |
| 273 | C |
| 274 | B |
| 275 | B |
| 276 | C |
| 277 | C |
| 278 | B |
| 279 | C |
| 280 | C |
| 281 | C |
| 282 | B |
| 283 | B |
| 284 | B |
| 285 | C |
| 287 | B |
| 288 | C |
| 289 | B |
| 291 | B |
| 292 | C |
| 293 | B |
| 294 | C |
| 295 | C |
| 296 | B |
| 297 | B |
| 299 | C |
| 300 | C |
| 301 | C |
| 302 | C |
| 303 | B |
| 304 | C |
| 305 | C |
| 306 | B |
| 307 | B |
| 310 | C |
| 311 | B |
| 312 | B |
| 313 | C |
| 314 | B |
| 315 | C |
| 316 | C |
| 317 | C |
| 318 | A |
| 319 | C |
| 320 | C |
| 321 | C |
| 322 | B |
| 323 | C |
| 324 | D |
| 325 | C |
| 326 | B |
| 327 | A |
| 328 | D |
| 329 | D |
| 330 | B |
| 331 | C |
| 334 | B |
| 335 | C |
| 336 | B |
| 337 | B |
| 338 | C |
| 339 | A |
| 342 | B |
| 343 | B |
| 344 | B |
| 345 | C |
| 346 | C |
| 347 | C |
| 348 | A |
| 349 | B |
| 350 | C |
| 351 | B |
| 353 | C |
| 354 | C |
| 355 | C |
| 356 | B |
| 357 | B |
| 358 | B |
| 359 | C |
| 360 | C |
| 362 | B |
| 363 | A |
| 364 | B |
| 365 | C |
| 366 | C |
| 370 | D |
| 373 | C |
| 374 | D |
| 375 | C |
| 376 | C |
| 378 | C |
| 379 | C |
| 380 | C |
| 383 | C |
| 384 | C |
| 385 | B |
| 386 | |
| 389 | C |
| 410 | C |
| 411 | C |
| 412 | C |
| 413 | B |
| 414 | C |
| 415 | B |
| 416 | C |
| 417 | C |
| 418 | B |
| 419 | B |
| 420 | B |
| 421 | B |
| 422 | B |
| 424 | B |
| 425 | C |
| 443 | B |
| 449 | B |
| 451 | C |
| 484 | C |
| 485 | C |
| 511 | B |
| 548 | D |
| 549 | D |
| 551 | B |
| 552 | B |
| 553 | C |
| 554 | B |
| 555 | B |
| 556 | B |
| 558 | B |
| 559 | B |
| 560 | C |
| 561 | B |
| 562 | C |
| 563 | C |
| 564 | C |
| 569 | C |
| 570 | C |
| 571 | C |
| 572 | B |
| 573 | C |
| 574 | B |
| 575 | C |
| 576 | B |
| 577 | D |
| 578 | B |
| 579 | C |
| 580 | B |
| 581 | B |
| 582 | C |

TABLE 14B
Cell viability data for
exemplary compounds

| Cmpnd | |
|---|---|
| No. | Viability |
| 904 | D |
| 905 | D |
| 906 | B |
| 908 | C |
| 909 | C |
| 912 | C |
| 913 | C |
| 914 | C |
| 915 | C |
| 916 | D |
| 917 | B |
| 918 | C |
| 919 | B |
| 920 | C |
| 921 | C |
| 922 | B |
| 923 | B |
| 924 | D |
| 925 | C |
| 926 | C |
| 927 | C |
| 928 | C |
| 929 | A |
| 930 | B |
| 931 | A |
| 932 | B |
| 933 | D |
| 934 | D |
| 935 | B |
| 936 | C |
| 937 | C |
| 938 | C |
| 939 | C |
| 940 | A |
| 941 | D |
| 942 | C |
| 943 | C |
| 944 | D |
| 945 | C |
| 946 | C |
| 948 | C |
| 949 | C |
| 950 | C |
| 951 | C |
| 954 | C |
| 955 | B |
| 956 | C |
| 957 | C |
| 958 | D |
| 959 | B |
| 960 | C |
| 961 | C |
| 962 | B |
| 963 | C |
| 964 | D |
| 965 | B |
| 966 | B |
| 967 | B |
| 968 | C |
| 969 | D |
| 970 | D |
| 971 | C |
| 978 | C |
| 979 | B |
| 980 | C |
| 981 | B |
| 982 | B |
| 983 | C |
| 984 | B |
| 986 | C |
| 987 | C |
| 988 | C |
| 989 | C |
| 991 | C |
| 992 | C |
| 993 | B |
| 994 | C |
| 995 | C |
| 996 | C |
| 997 | D |
| 998 | B |
| 999 | C |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

The invention claimed is:

1. A compound of Formula (I): 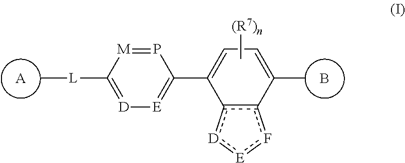
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L is absent, C₁-C₆-alkylene, C₁-C₆-heteroalkylene, —O—, —S—, —C(O)—, —N(R³)—, —N(R³)C(O)—, or —C(O)N(R³)—, wherein each alkylene and heteroalkylene is optionally substituted with one or more R⁴;
M and P are each independently C(R²) or N;
D, E, and F are each independently C(R^{5a}), C(R^{5a})(R^{5b}), N, N(R^{5c}), S, or O, wherein at least one of D, E, and F is each independently N or N(R^{5c}), and the bonds between the atoms in the ring comprising D, E, and F may be single bonds or double bonds as valency permits;
each R¹ is independently hydrogen, C₁-C₆-alkyl, C₂-C₆-alkenyl, C2-C6-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸;
each R² is independently hydrogen, C₁-C₆-alkyl, C₂-C₆-alkenyl, C2-C6-alkynyl, C₁-C₆-heteroalkyl, C₁-C₆-haloalkyl, cycloalkyl, heterocyclyl, halo, cyano, or —OR^{A};
each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²;
each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
R^{5a} and R^{5b} are each independently hydrogen, halo, C₁-C₆ alkyl, or cycloalkyl; or
R^{5a} and R^{5b} are taken together to form an oxo group;
R^{5c} is hydrogen, C₁-C₆ alkyl, or cycloalkyl;
each R⁷ is independently C₁-C₆-alkyl, C₂-C₆-alkenyl, C₂-C₆-alkynyl, C₁-C₆-heteroalkyl, C₁-C₆-haloalkyl, halo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹; or
two R⁷ groups, together with the atoms to which they are attached form a 4-7-membered cycloalkyl, heterocyclyl, aryl, or heteroaryl, wherein each cycloalkyl, heterocyclyl, aryl, and heteroaryl is optionally substituted with one or more R⁹;
R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D};
R^{B} and R^{C} together with the atom to which they are attached form a 3-7-membered heterocyclyl or heteroaryl ring optionally substituted with one or more R¹⁰;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl;
each R¹⁰ is independently C₁-C₆-alkyl, C₁-C₆-heteroalkyl, C₁-C₆-haloalkyl, halo, or cycloalkyl;
each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};
each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};
n is 0, 1, or 2; and
x is 1, or 2.

2. The compound of claim 1, wherein each of A and B is independently heteroaryl or heterocyclyl, each of which is optionally substituted with one or more R¹.

3. The compound of any one of the preceding claims, wherein one of A and B is independently a monocyclic heteroaryl or bicyclic heteroaryl, each of which is optionally substituted with one or more R¹.

4. The compound of any one of the preceding claims, wherein one of A and B is independently a monocyclic heteroaryl optionally substituted with one or more R¹.

5. The compound of any one of the preceding claims, wherein one of A and B is independently a nitrogen-containing heteroaryl optionally substituted with one or more R¹.

6. The compound of any one of the preceding claims, wherein one of A and B is independently a 5-6 membered heteroaryl optionally substituted with one or more R¹.

7. The compound of any one of the preceding claims, wherein one of A and B is independently selected from 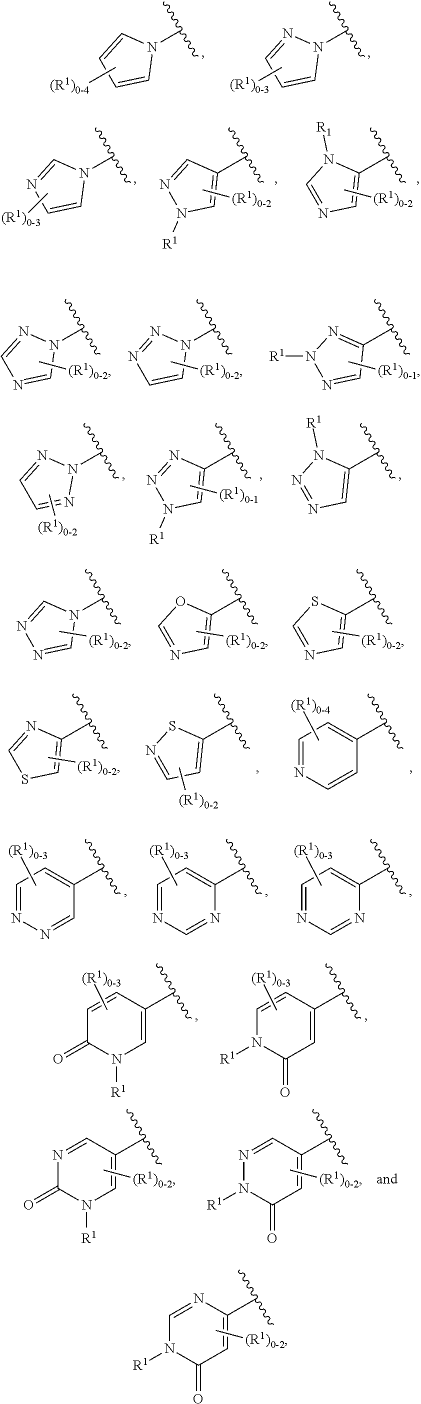
wherein R¹ is as described in claim 1.

8. The compound of any one of the preceding claims, wherein one of A and B is independently selected from 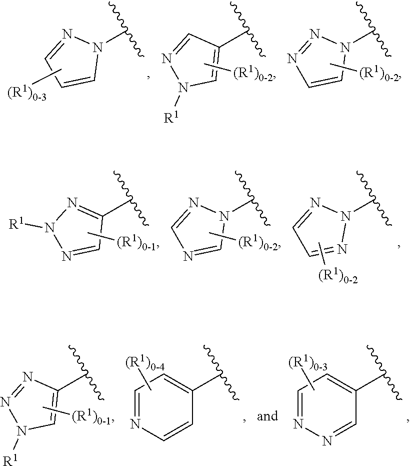
wherein R¹ is as described in claim 1.

9. The compound of any one of the preceding claims, wherein one of A and B is independently selected from 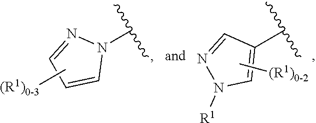
wherein R¹ is as described in claim 1.

10. The compound of any one of the preceding claims, wherein A is selected from 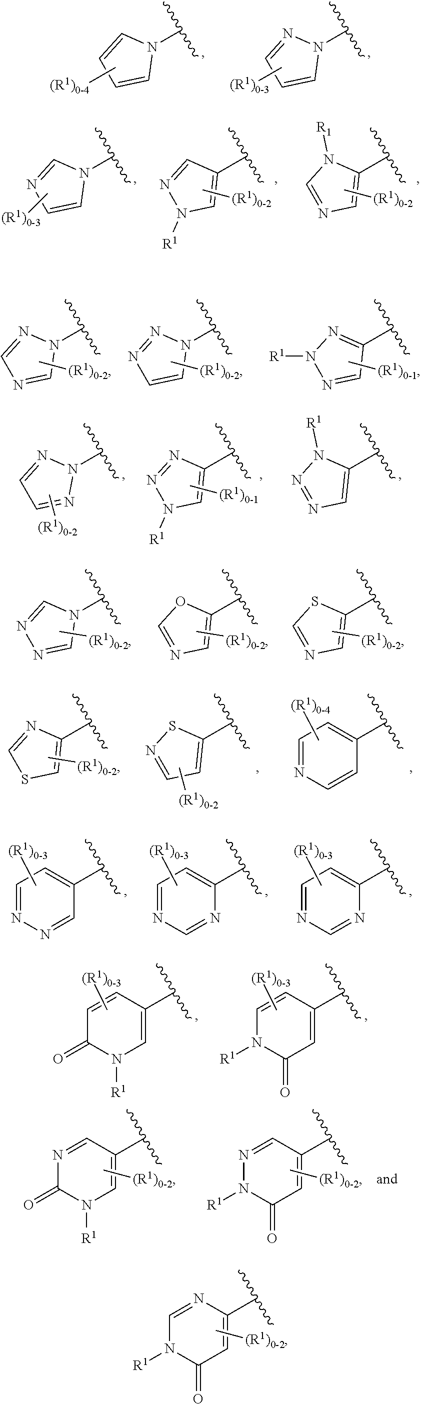
wherein R¹ is as described in claim 1.

11. The compound of any one of the preceding claims, wherein B is selected from 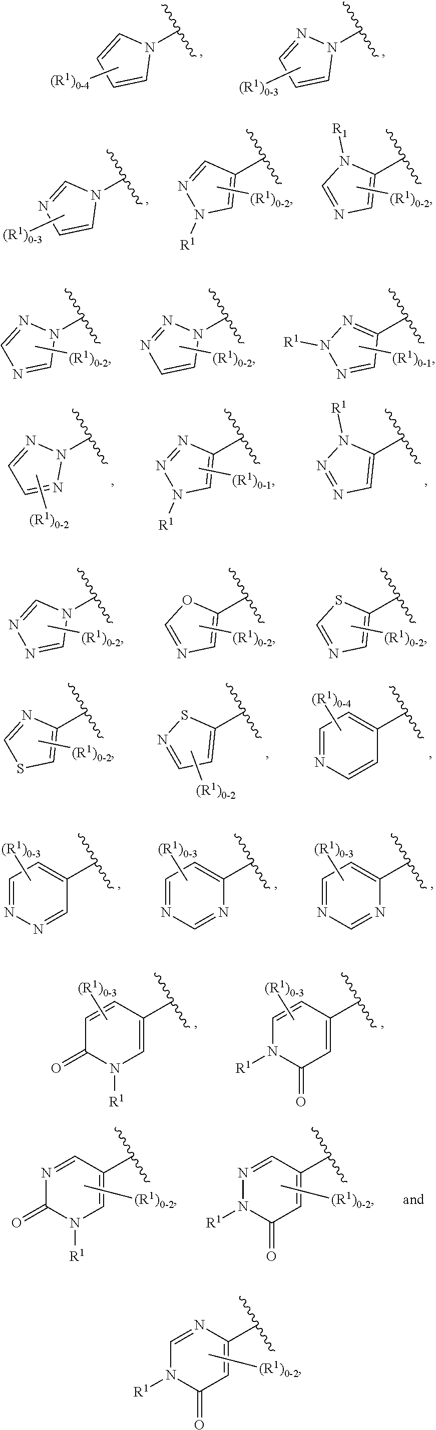
wherein R¹ is as described in claim 1.

12. The compound of any one of the preceding claims, B is selected from 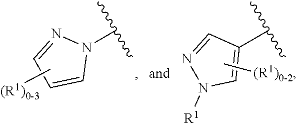
wherein R¹ is as described in claim 1.

13. The compound of any one of the preceding claims, wherein one of A and B is
independently selected from

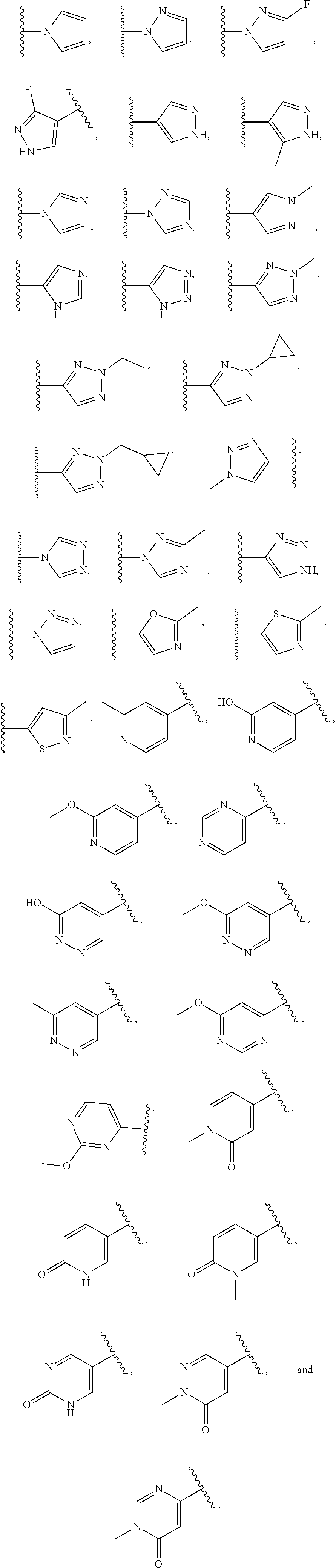

14. The compound of any one of the preceding claims, wherein one of A and B is independently selected from

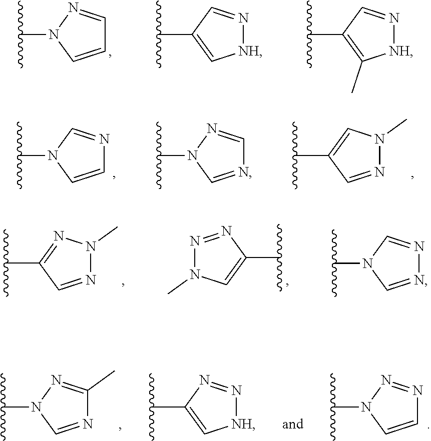

15. The compound of any one of the preceding claims, wherein one of A and B is independently selected from

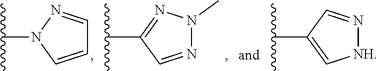

16. The compound of any one of the preceding claims, wherein A is selected from

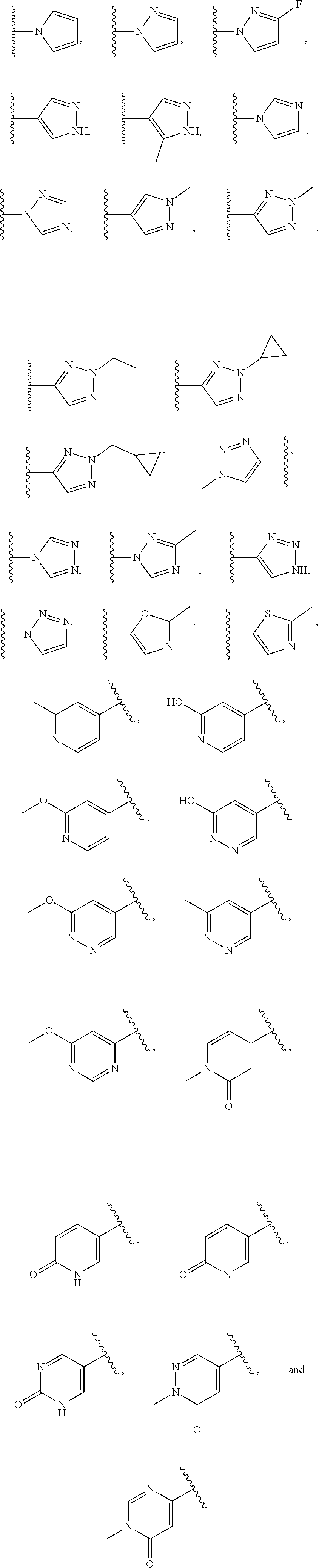

17. The compound of any one of the preceding claims, wherein B is selected from

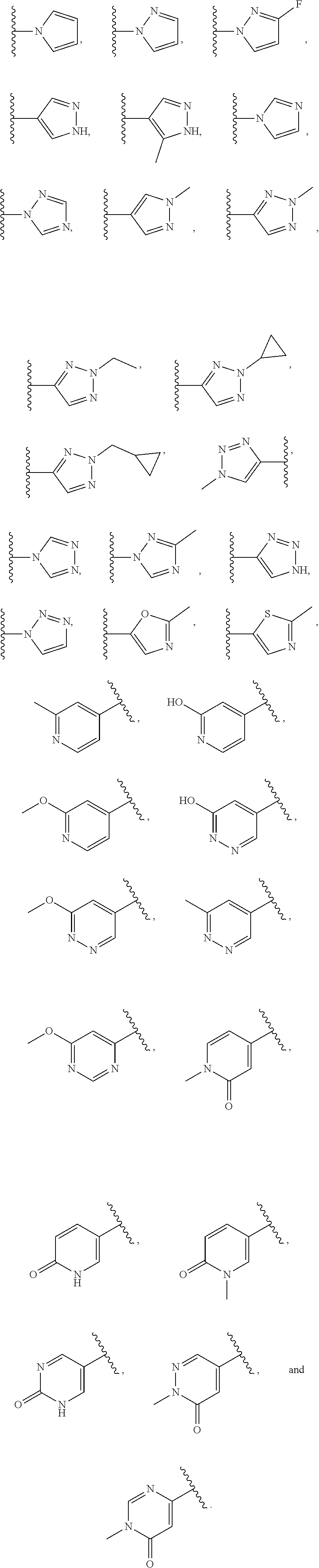

18. The compound of any one of the preceding claims, wherein B is selected from

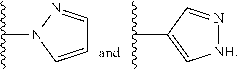 [SMILES: *n1cccn1 ; *c1cn[nH]c1]

19. The compound of any one of the preceding claims, wherein one of A and B is independently a monocyclic heterocyclyl or bicyclic heterocyclyl, each of which is optionally substituted with one or more R¹.

20. The compound of any one of the preceding claims, wherein one of A and B is independently a nitrogen-containing heterocyclyl optionally substituted with one or more R¹.

21. The compound of any one of the preceding claims, wherein one of A and B is independently a 4-10 membered heterocyclyl optionally substituted with one or more R¹.

22. The compound of any one of the preceding claims, wherein one of A and B is independently selected from 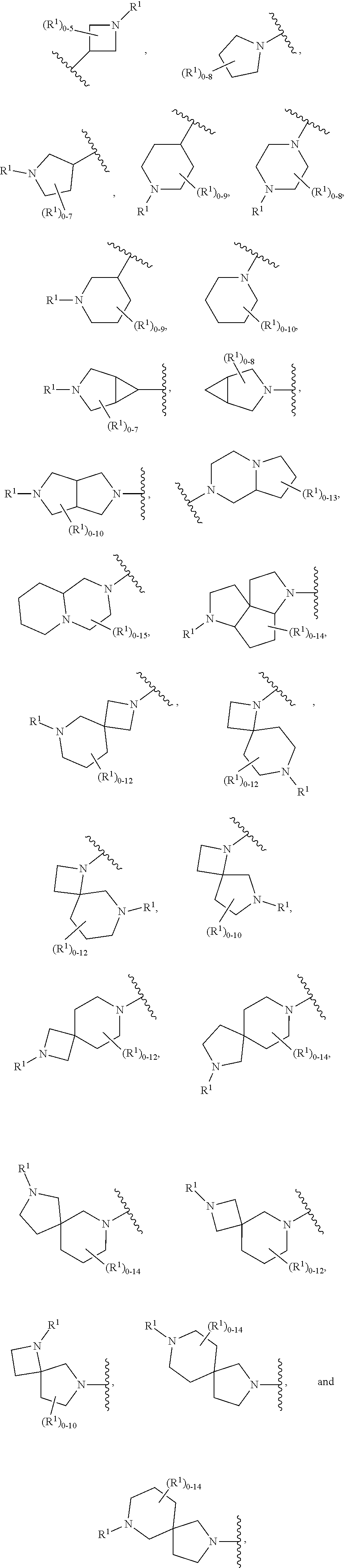
wherein R¹ is as described in claim 1.

23. The compound of any one of the preceding claims, wherein one of A and B is independently selected from 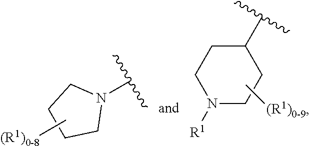
wherein R¹ is as described in claim 1.

24. The compound of any one of the preceding claims, wherein one of A and B is 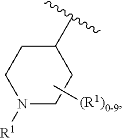
wherein R¹ is as described in claim 1.

25. The compound of any one of the preceding claims, wherein A is selected from 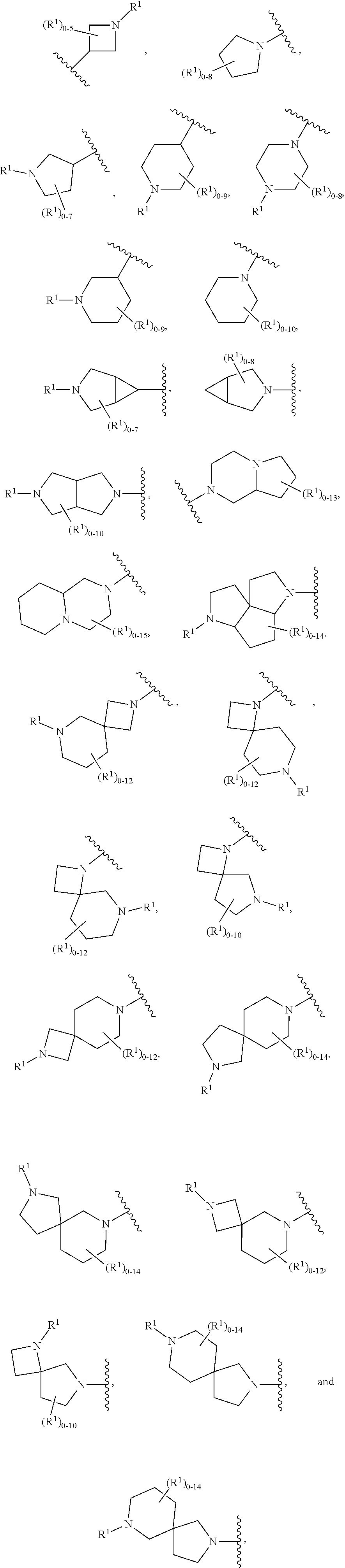
wherein R¹ is as described in claim 1.

26. The compound of any one of the preceding claims, wherein B is selected from 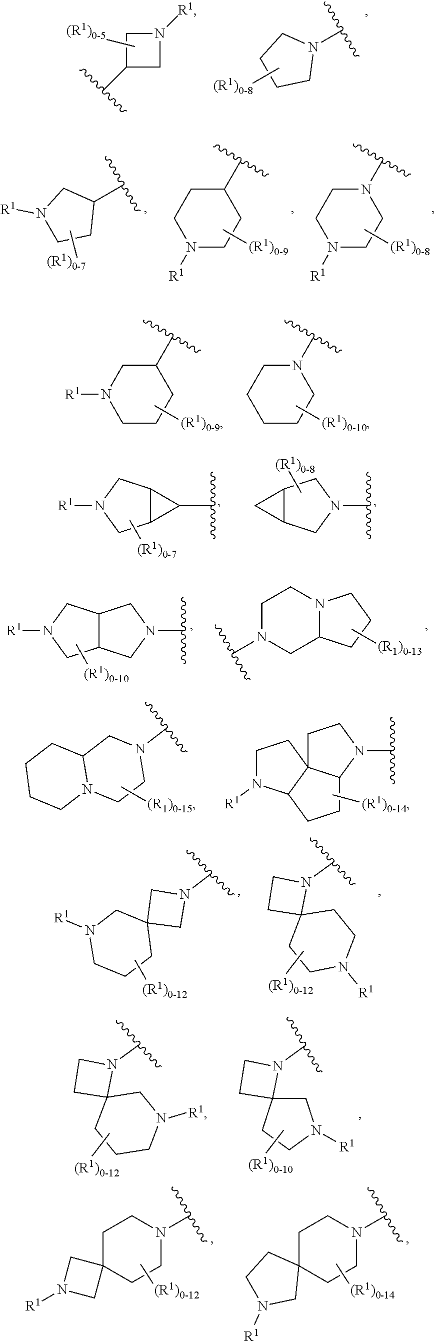 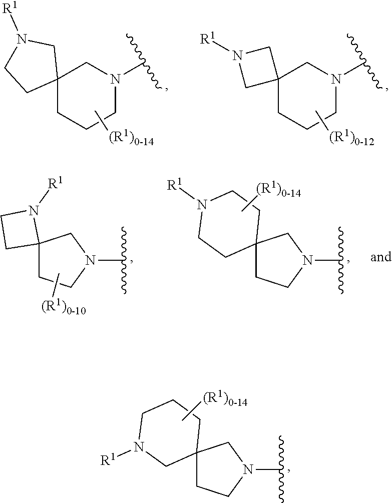
wherein R¹ is as described in claim 1.

27. The compound of any one of the preceding claims, wherein A is 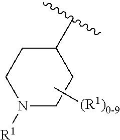
and R¹ is as described in claim 1.

28. The compound of any one of the preceding claims, wherein one of A and B is independently selected from

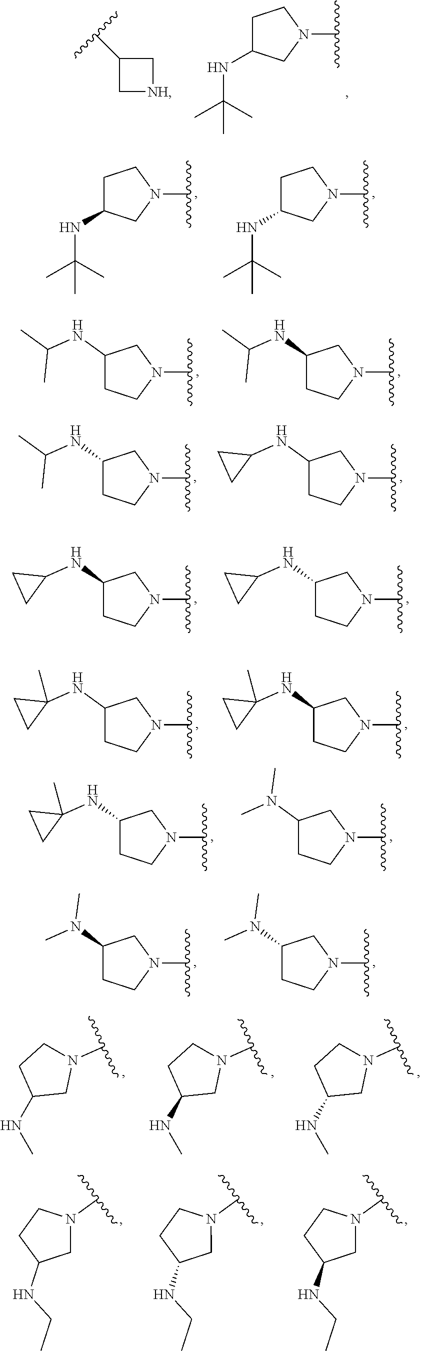

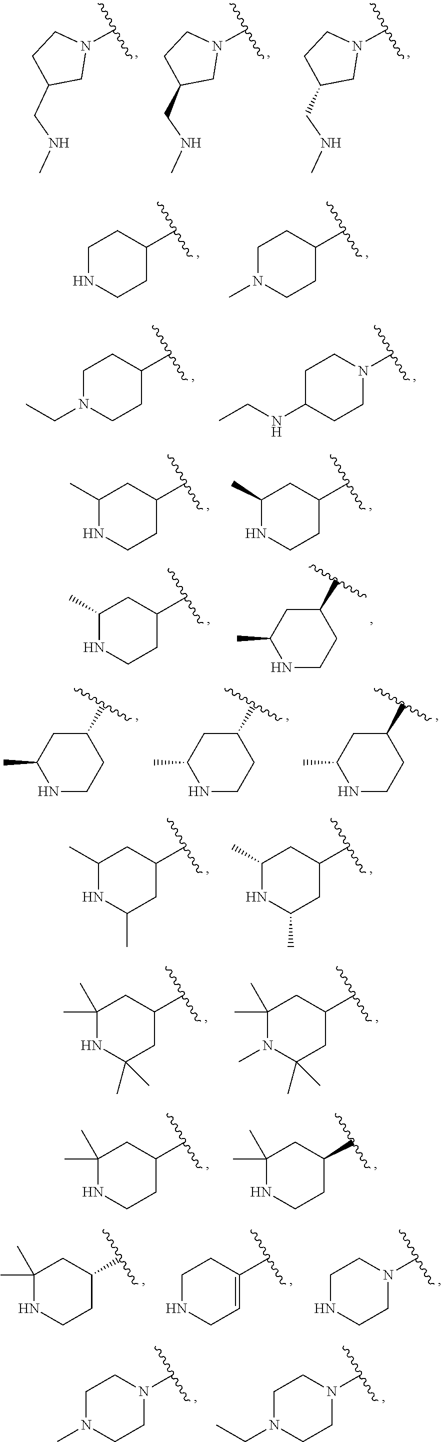

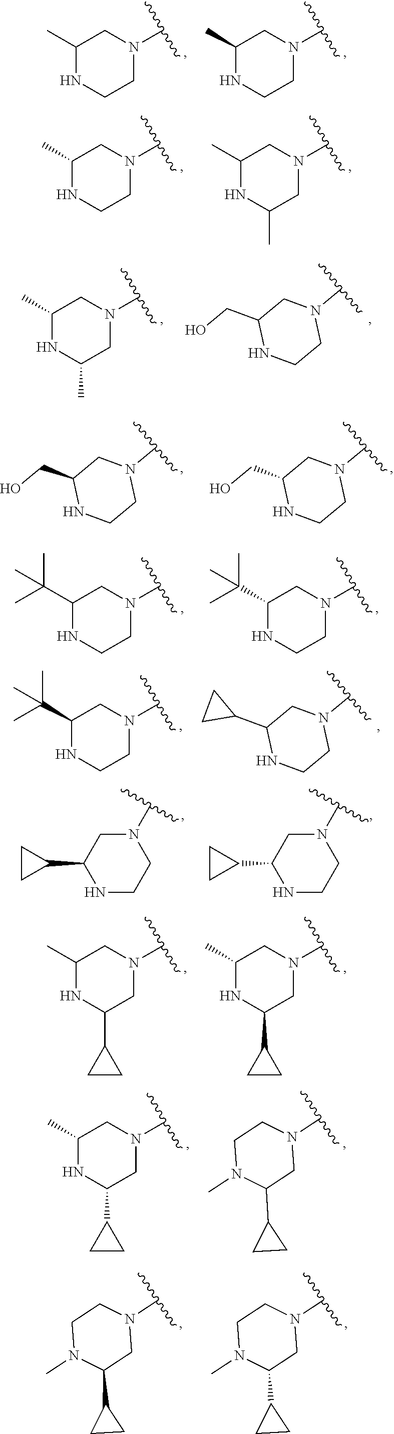

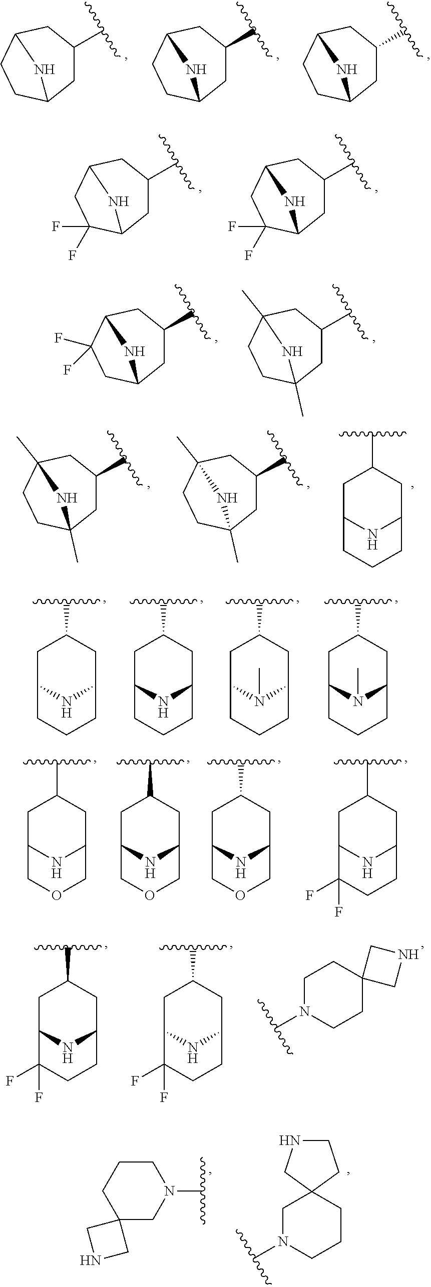

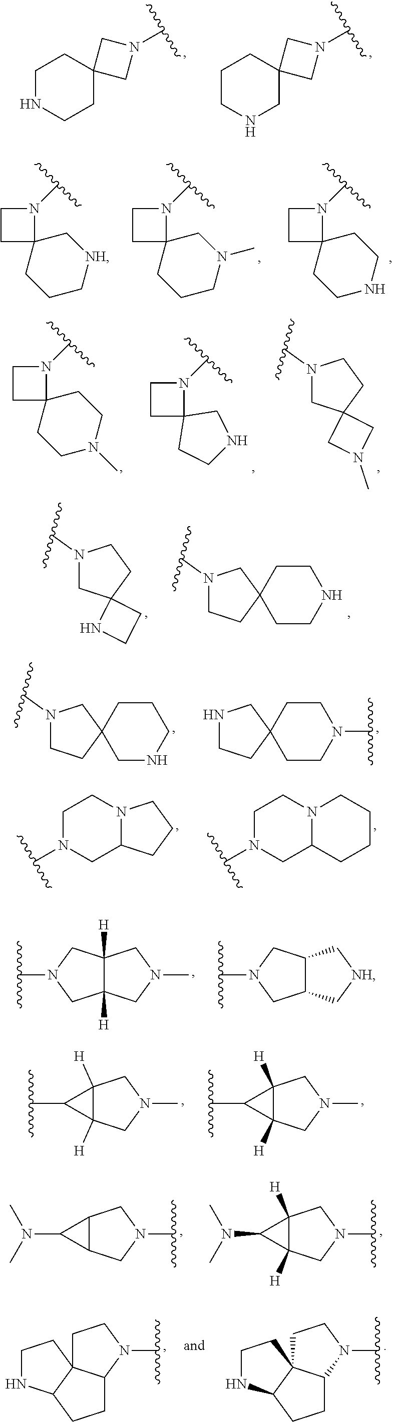

29. The compound of any one of the preceding claims, wherein one of A and B is independently selected from

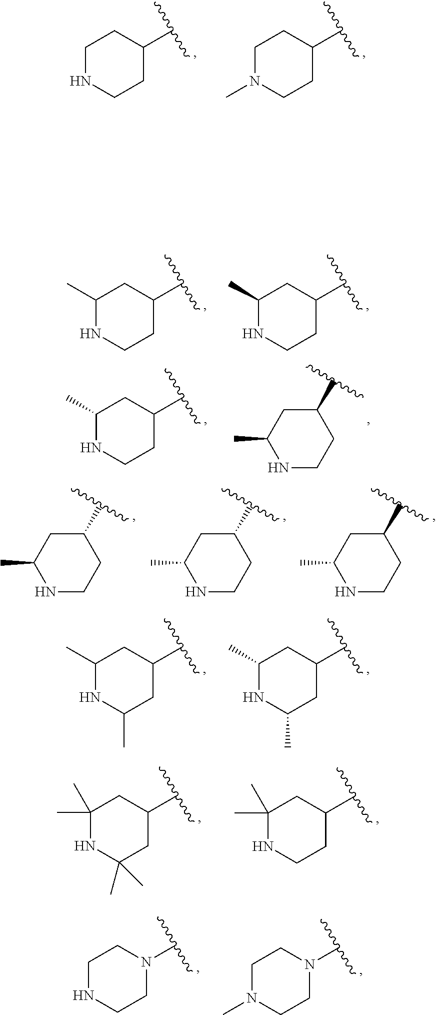

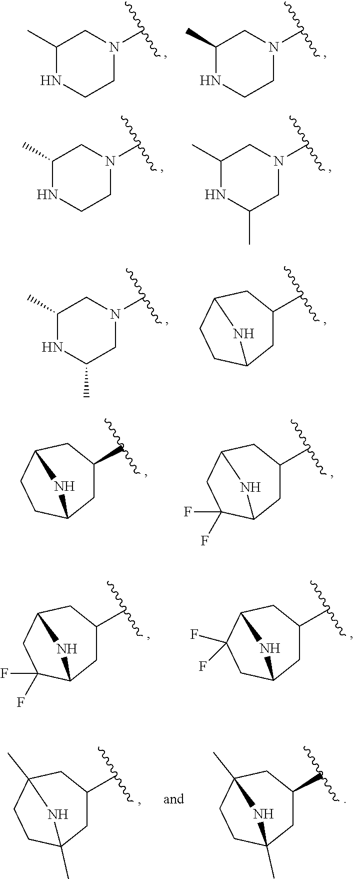

30. The compound of any one of the preceding claims, wherein one of A and B is independently selected from

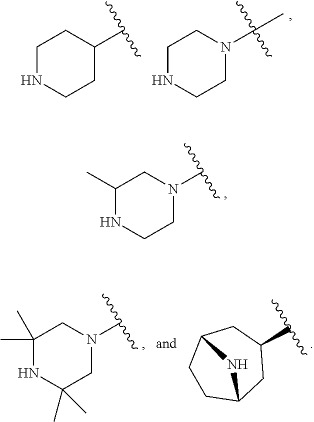

31. The compound of any one of the preceding claims, wherein one of A and B is independently selected from

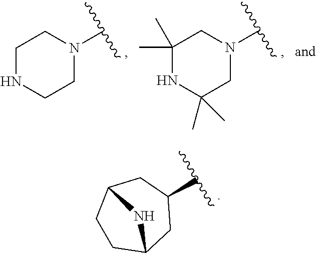

32. The compound of any one of the preceding claims, wherein A is selected from

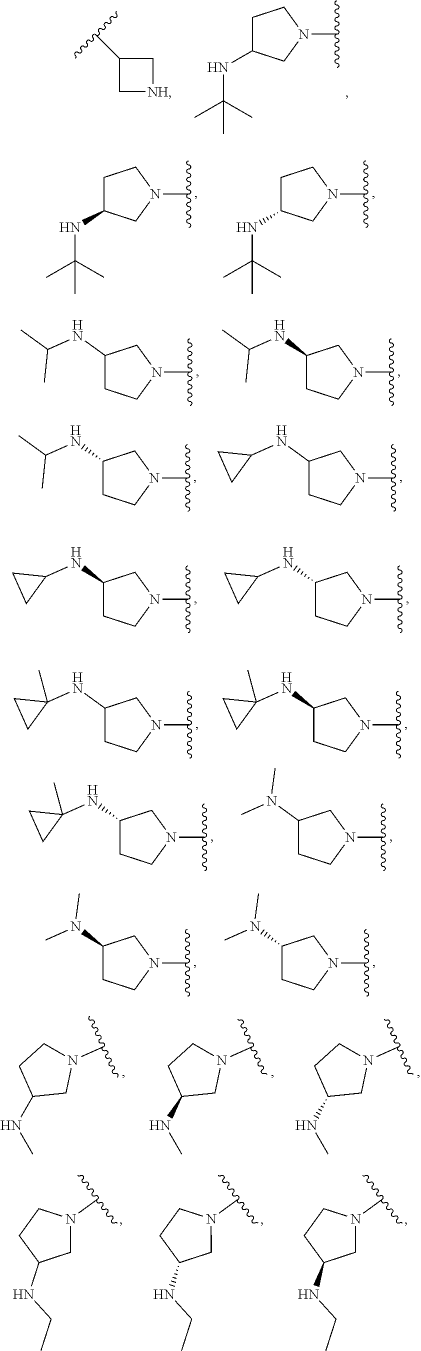

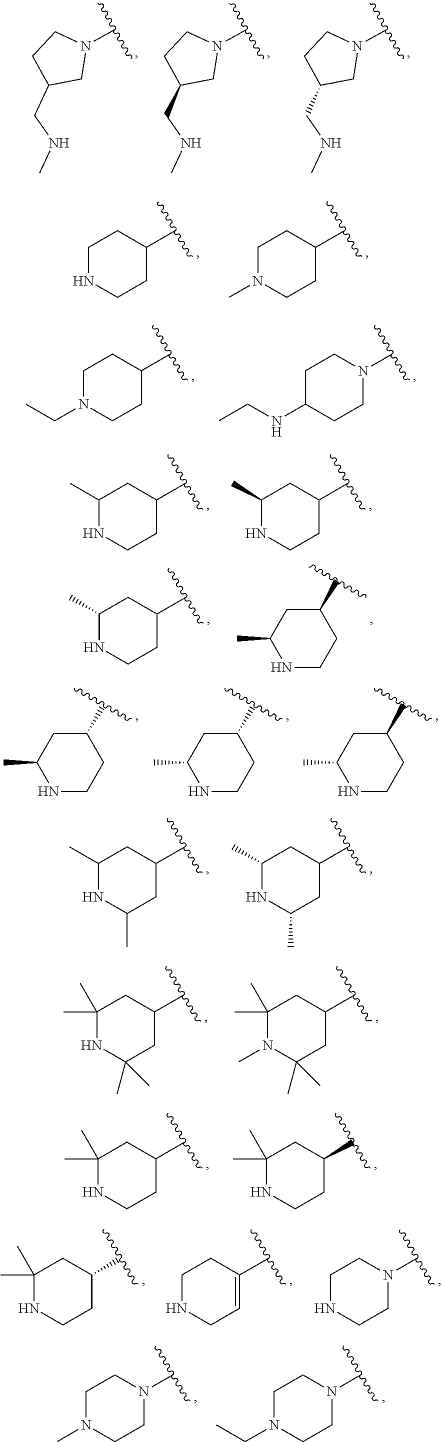

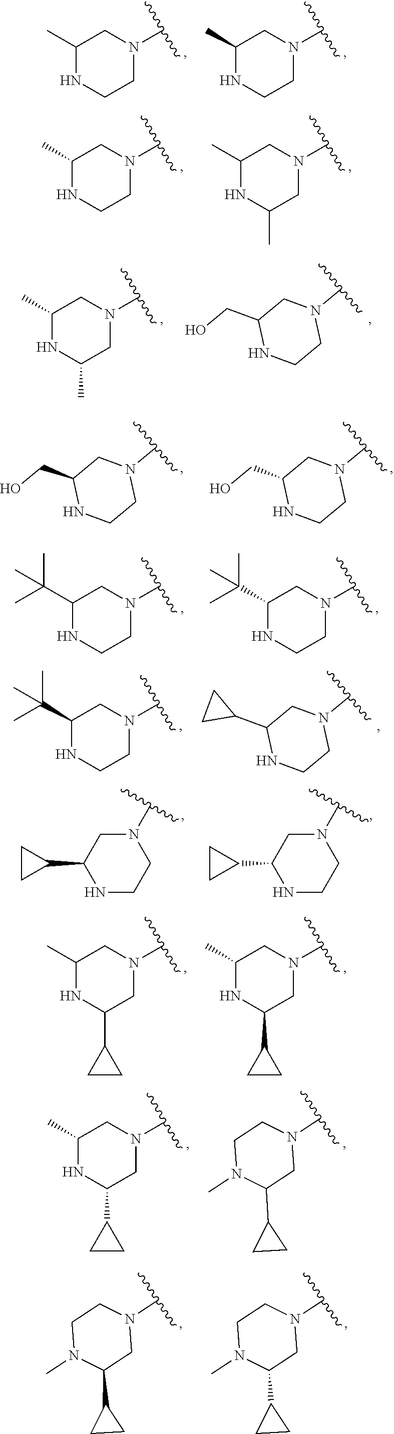

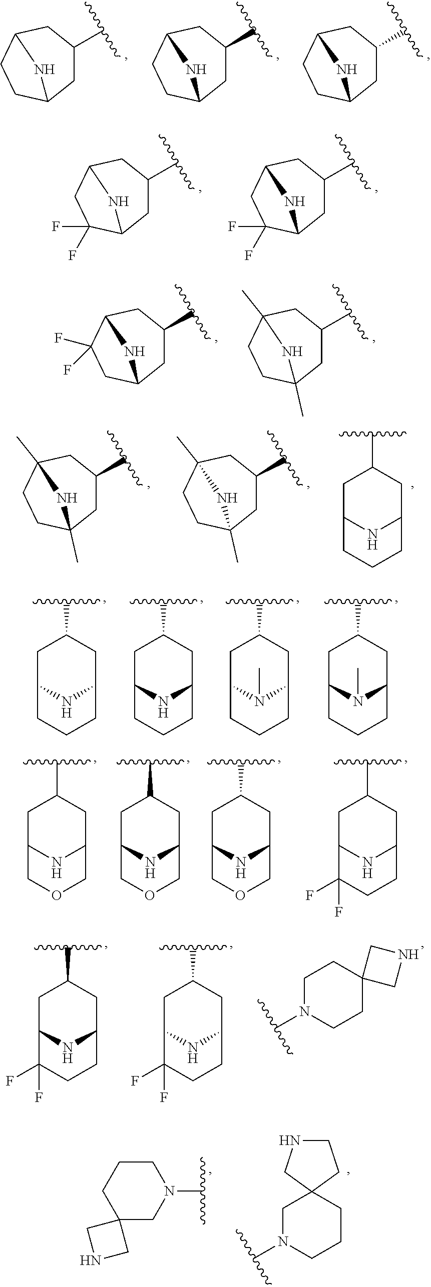

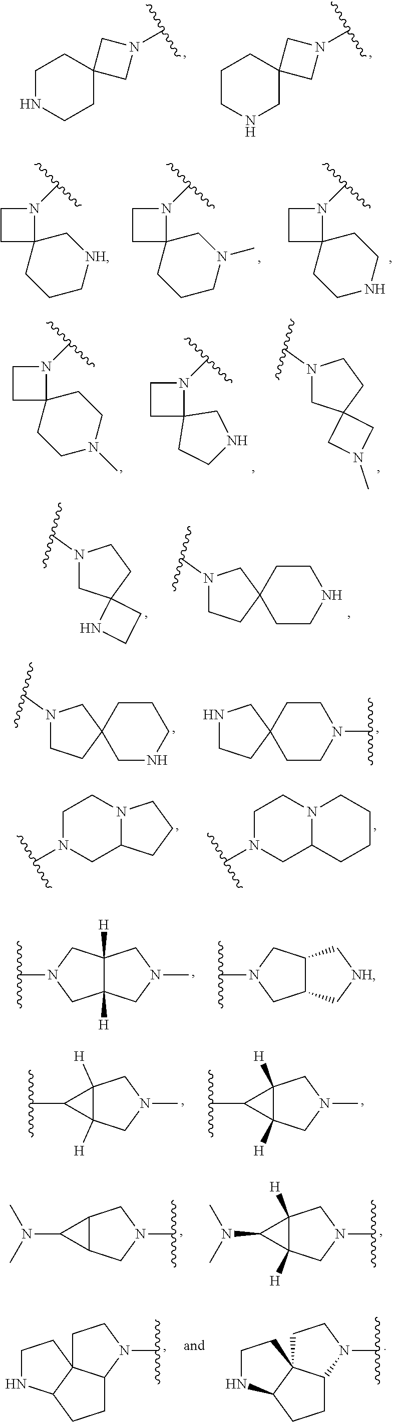

33. The compound of any one of the preceding claims, wherein B is selected from

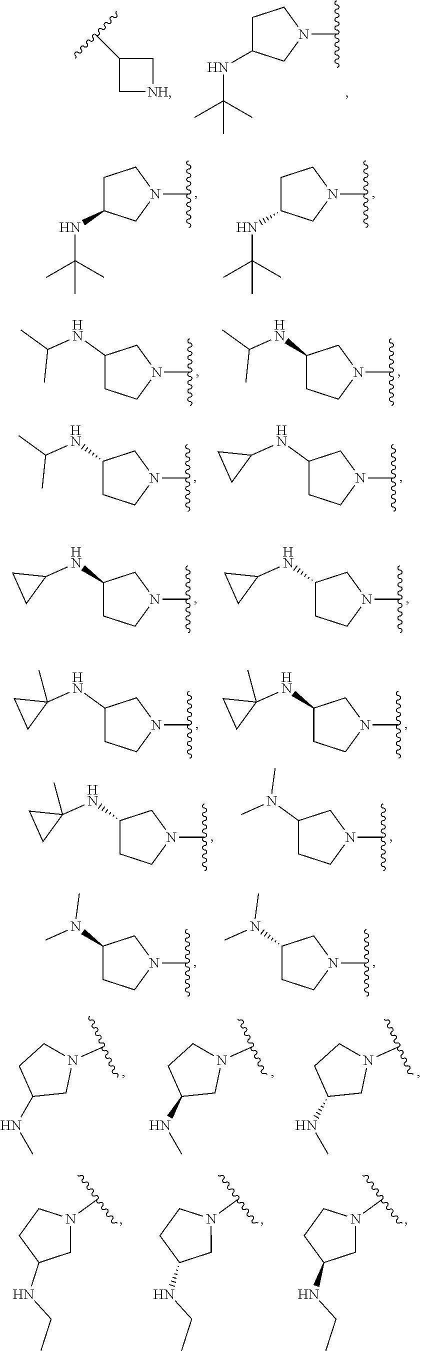

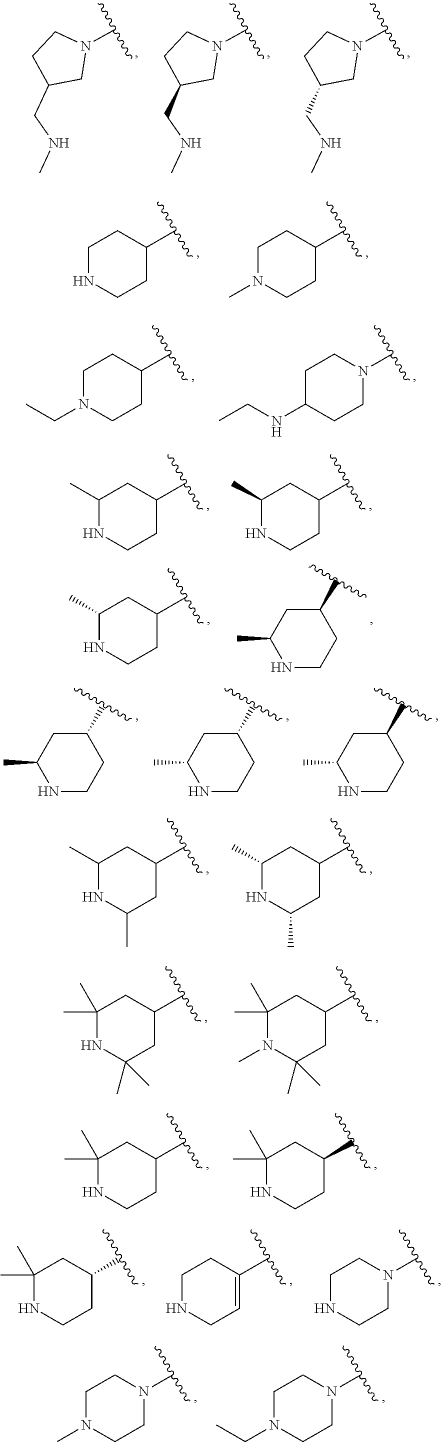

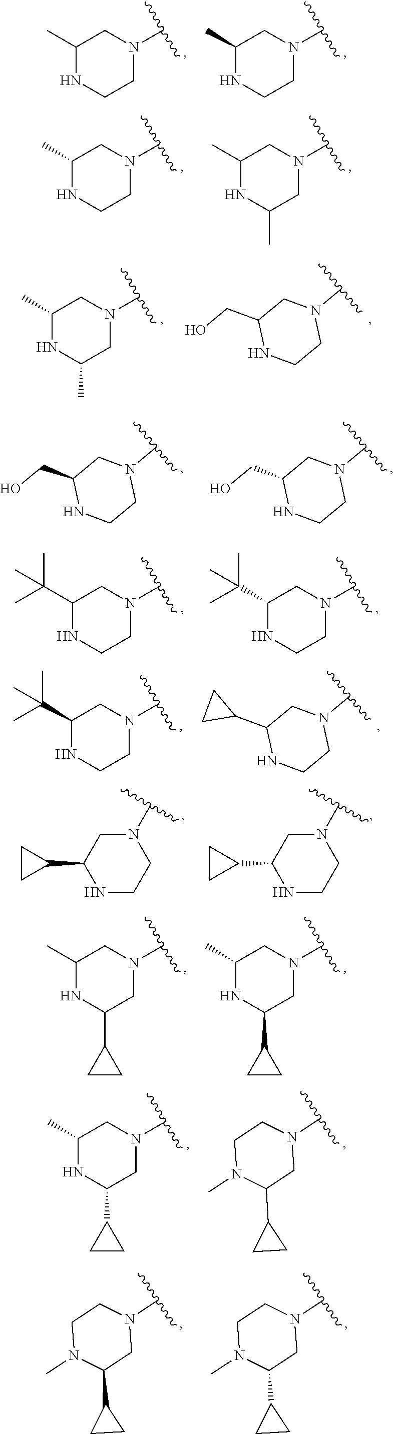

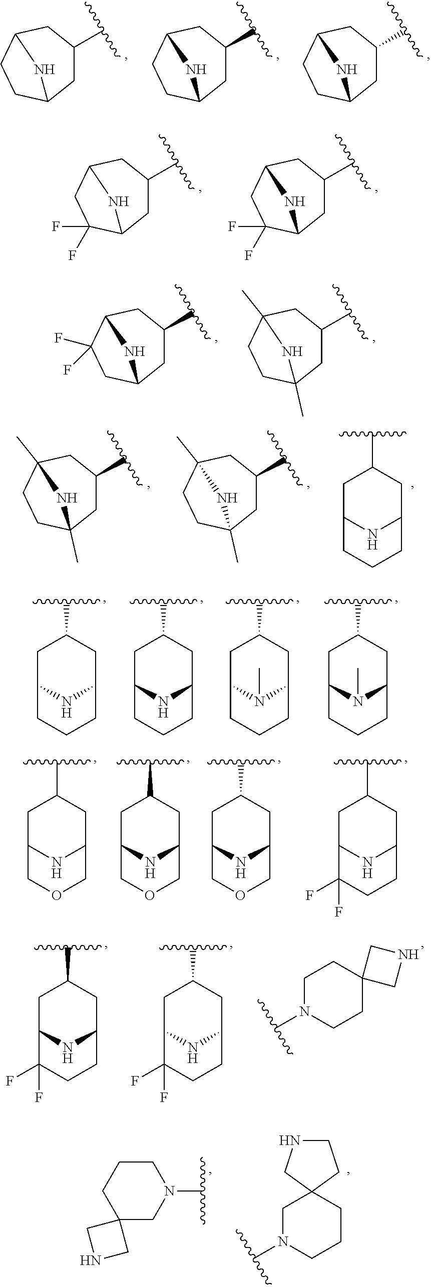

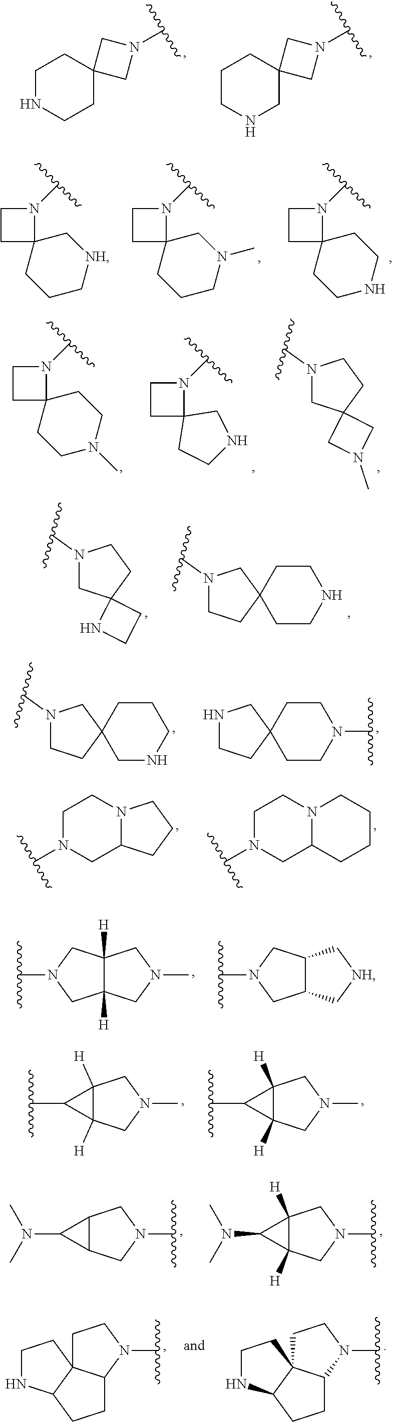

34. The compound of any one of the preceding claims, wherein A is selected from

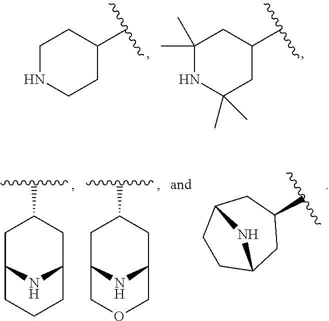

35. The compound of any one of the preceding claims, wherein L is absent, —O—, —S—, or —N(R³)—.

36. The compound of any one of the preceding claims, wherein L is absent.

37. The compound of any one of the preceding claims, wherein L is —O— or —N(R³)—.

38. The compound of any one of the preceding claims, wherein L is —N(R³)— and R³ is hydrogen, C₁-C₆-alkyl, or cycloalkyl, wherein each alkyl and heteroalkyl are each optionally substituted with one or more R¹² (e.g., deuterium).

39. The compound of any one of the preceding claims, wherein L is —N(CH₃)— or —N(H)—.

40. The compound of any one of the preceding claims, wherein L is —N(CH₃)—.

41. The compound of any one of the preceding claims, wherein 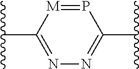
is selected from

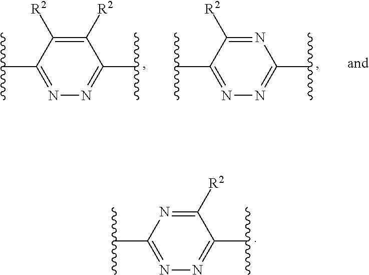

42. The compound of any one of the preceding claims, wherein

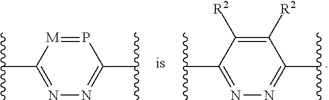

43. The compound of any one of claims 1-41, wherein

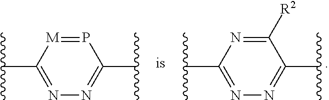

44. The compound of any one of the preceding claims, wherein each of M and P is independently C(R²) (e.g., CH).

45. The compound of any one of claims 1-43, wherein M is N and P is C(R²) (e.g., CH).

46. The compound of any one of the preceding claims, wherein two of D, E, and F is independently N, N(R⁵), or S.

47. The compound of any one of the preceding claims, wherein D is N, N(R^{5c}), or S.

48. The compound of any one of the preceding claims, wherein D is N or N(R^{5c}) (e.g., NH).

49. The compound of any one of the preceding claims, wherein each of D and E is independently N or N(R^{5c}).

50. The compound of any one of the preceding claims, wherein F is N.

51. The compound of any one of the preceding claims, wherein 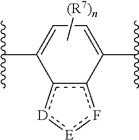
is selected from

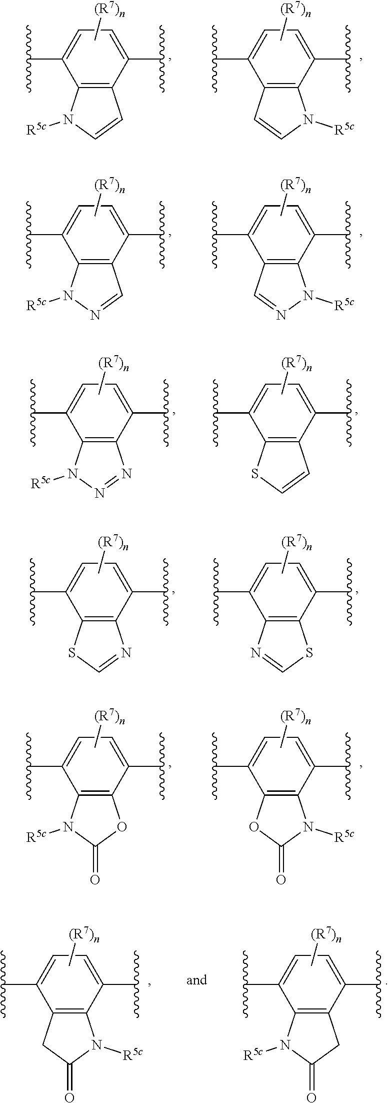

52. The compound of any one of the preceding claims, wherein 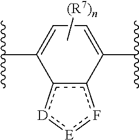
is selected from

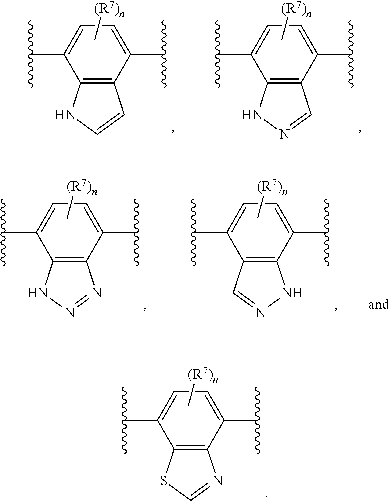

53. The compound of any one of the preceding claims, wherein 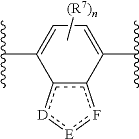
is selected from

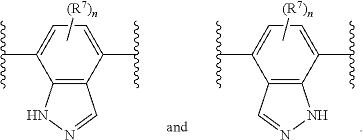

54. The compound of any one of the preceding claims, wherein 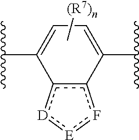
is from

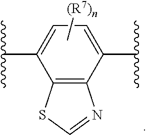

55. The compound of any one of the preceding claims, wherein

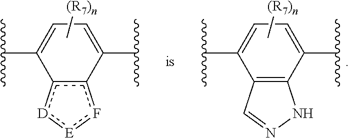

56. The compound of any one of the preceding claims, wherein the compound is a compound of Formula (I-a): 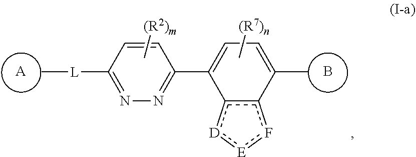
or a pharmaceutically acceptable salt, solvate, hydrate, tautomer, or stereoisomer thereof, wherein A, B, L, D, E, F, R², R⁷, m, n, and subvariables thereof are as described in claim 1.

57. The compound of any one of the preceding claims, wherein the compound is a compound of Formula (I-b): 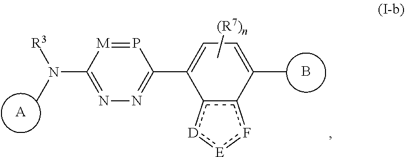
or a pharmaceutically acceptable salt, solvate, hydrate, tautomer, or stereoisomer thereof, wherein A, B, M, P, D, E, F, R³, R⁷, n, and subvariables thereof are as described in claim 1.

58. The compound of any one of the preceding claims, wherein the compound is a compound of Formula (I-c): 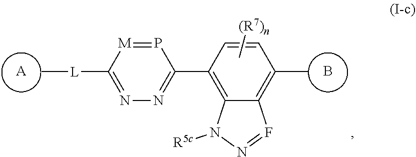
or a pharmaceutically acceptable salt, solvate, hydrate, tautomer, or stereoisomer thereof, wherein A, B, M, P, L, F, R^{5c}, R⁷, n, and subvariables thereof are as described in claim 1.

59. The compound of any one of the preceding claims, wherein the compound is a compound of Formula (I-d): 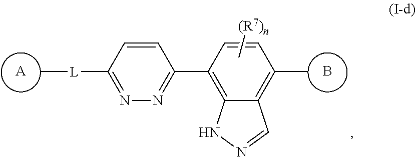
or a pharmaceutically acceptable salt, solvate, hydrate, tautomer, or stereoisomer thereof, wherein A, B, L, R⁷, n, and subvariables thereof are as described in claim 1.

60. The compound of any one of the preceding claims, wherein the compound is a compound of Formula (I-e): 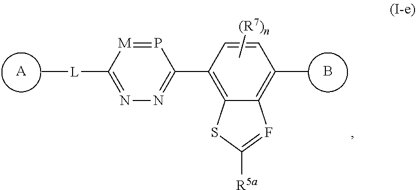
or a pharmaceutically acceptable salt, solvate, hydrate, tautomer, or stereoisomer thereof, wherein A, B, M, P, L, F, R^{5a}, R⁷, n, and subvariables thereof are as described in claim 1.

61. The compound of any one of the preceding claims, wherein the compound is a compound of Formula (I-f): 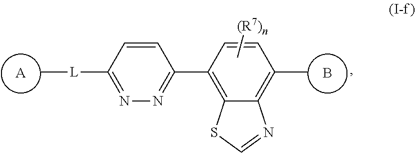
or a pharmaceutically acceptable salt, solvate, hydrate, tautomer, or stereoisomer thereof, wherein A, B, L, R⁷, n, and subvariables thereof are as described in claim 1.

62. The compound of any one of the preceding claims, wherein the compound is a compound of Formula (I-g): 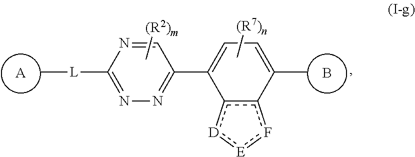
or a pharmaceutically acceptable salt, solvate, hydrate, tautomer, or stereoisomer thereof, wherein A, B, L, D, E, F, R², R⁷, m, n, and subvariables thereof are as described in claim 1.

63. The compound of any one of the preceding claims, wherein the compound is a compound of Formula (I-h): 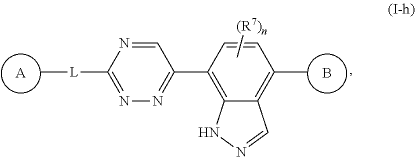
or a pharmaceutically acceptable salt, solvate, hydrate, tautomer, or stereoisomer thereof, wherein A, B, L, R⁷, n, and subvariables thereof are as described in claim 1.

64. The compound of claim 1, wherein:
A is a monocyclic, bicyclic, or tricyclic nitrogen-containing heterocyclyl;
B is a monocyclic nitrogen-containing heteroaryl;
L is absent, —N(R³)—, or —O—;
M is CH;
P is CH;
D is N(R^{5a}) or S;
E and F is each independently N, NH, C(R^{5a}), C(R^{5a})(R^{5b});
R⁷ is halo; and
n is 0 or 1.

65. The compound of any one of the preceding claims, wherein the compound is selected from any one of the compounds shown in Table 1 or a pharmaceutically acceptable salt, solvate, hydrate, tautomer, or stereoisomer thereof.

66. A compound of Formula (II): 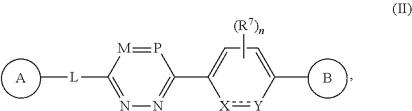
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L is absent, —O—, —C(O)—, —N(R³)—;
M and P are each independently C(R²) or N;
X and Y are each independently C(R^{5a}), C(R^{5a})(R^{5b}), N, or N(R^{5c}), wherein the bond between X and Y may be a single or double bond as valency permits, and wherein X and Y may not both be C(R^{5a})(R^{5b}) or C(R^{5a});
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸;
each R² is independently hydrogen, halo, cyano, C₁-C₆-alkyl, C₂-C₆-alkenyl, C₂-C₆-alkynyl, or —OR^{A};
each R³ is independently hydrogen, C₁-C₆-alkyl, or C₁-C₆-haloalkyl;
each of R^{5a} and R^{5b} is C₁-C₆-alkyl, C₂-C₆-alkenyl, C₂-C₆-alkynyl, C₁-C₆-heteroalkyl, C₁-C₆-haloalkyl, cycloalkyl, halo, cyano, oxo, —OR^{A1}, —NR^{B}R^{C}, —C(O)R^{D}, or —C(O)OR^{D};
R^{5a} and R^{5b}, together with the carbon atom to which they are attached, form an oxo group;
each R^{5c} is hydrogen, C₁-C₆-alkyl, C₁-C₆-haloalkyl, cycloalkyl, or C(O)R^{D};
each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹;
R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D};
R^{B} and R^{C} together with the atom to which they are attached form a 3-7-membered heterocyclyl or heteroaryl ring optionally substituted with one or more R¹⁰;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl;
R¹⁰ is C₁-C₆-alkyl or halo;
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A1} is independently C₁-C₆ alkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D};
n is 0, 1, or 2; and
x is 0, 1, or 2.

67. The compound of claim 66, wherein each of A and B is independently heteroaryl or heterocyclyl, each of which is optionally substituted with one or more R¹.

68. The compound of any one of the preceding claims, wherein one of A and B is independently a monocyclic heteroaryl or bicyclic heteroaryl, each of which is optionally substituted with one or more R¹.

69. The compound of any one of the preceding claims, wherein one of A and B is independently a monocyclic heteroaryl optionally substituted with one or more R¹.

70. The compound of any one of the preceding claims, wherein one of A and B is independently a nitrogen-containing heteroaryl optionally substituted with one or more R¹.

71. The compound of any one of the preceding claims, wherein one of A and B is independently a 5-6 membered heteroaryl optionally substituted with one or more R¹.

72. The compound of any one of the preceding claims, wherein one of A and B is independently selected from 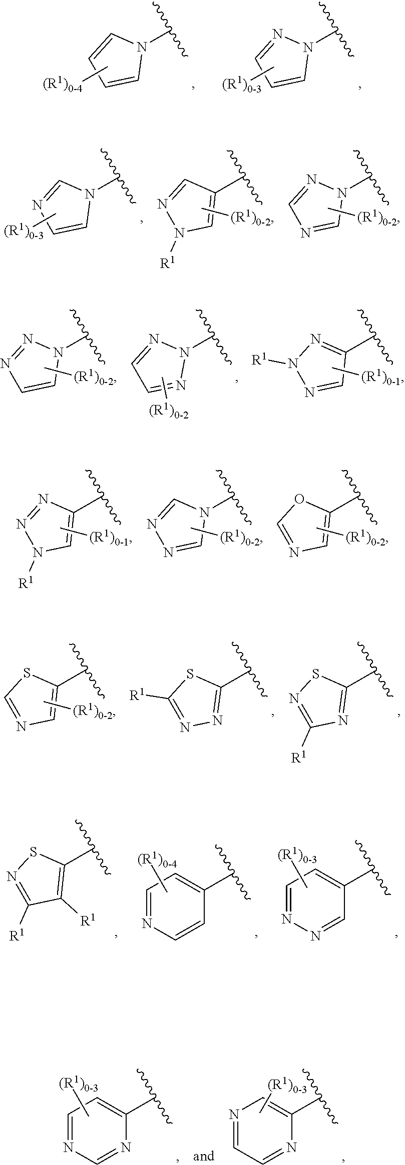
wherein R¹ is as described in claim 66.

73. The compound of any one of the preceding claims, wherein one of A and B is independently selected from 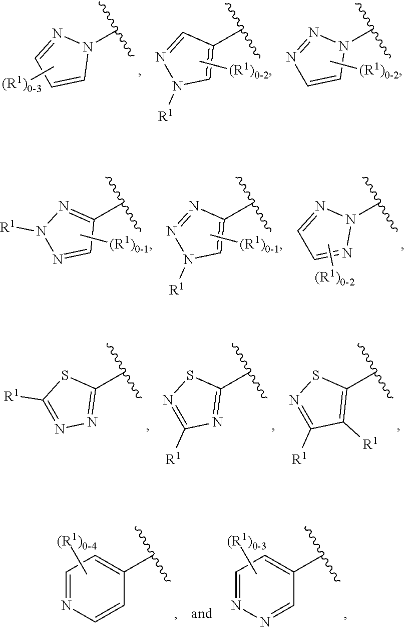
wherein R¹ is as described in claim 66.

74. The compound of any one of the preceding claims, wherein one of A and B is independently selected from 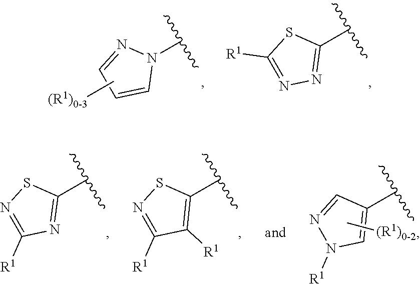
wherein R¹ is as described in claim 66.

75. The compound of any one of the preceding claims, wherein one of A and B is independently selected from 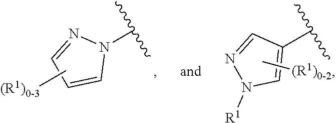
wherein R¹ is as described in claim 66.

76. The compound of any one of the preceding claims, wherein A is selected from 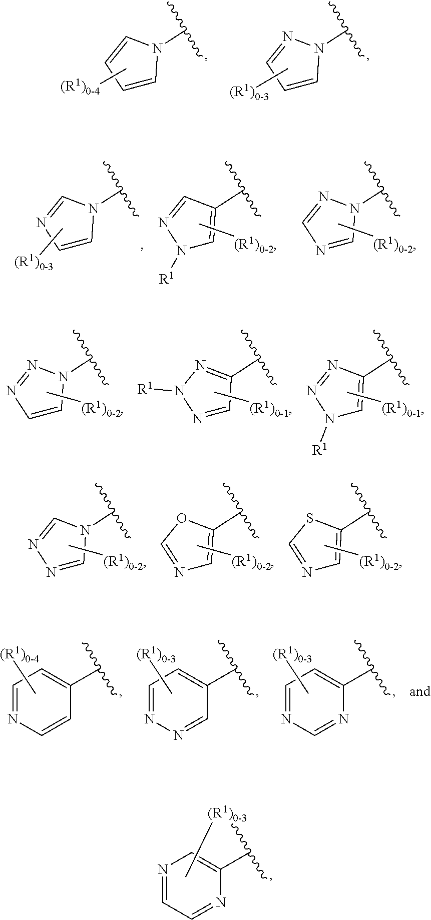
wherein R¹ is as described in claim 66.

77. The compound of any one of the preceding claims, wherein B is selected from 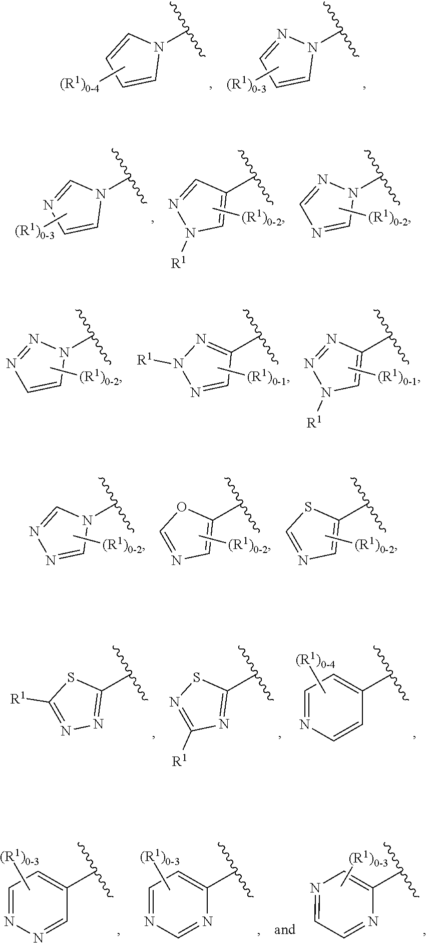
wherein R¹ is as described in claim 66.

78. The compound of any one of the preceding claims, B is selected from 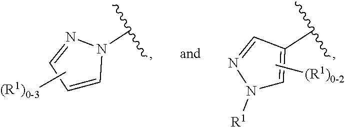
wherein R¹ is as described in claim 66.

79. The compound of any one of the preceding claims, wherein one of A and B is independently selected from

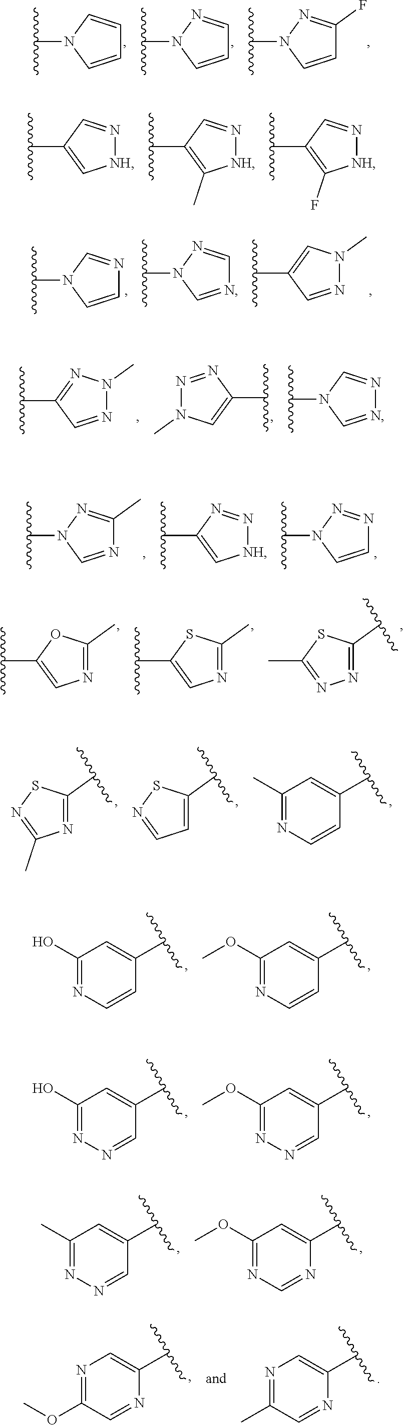

80. The compound of any one of the preceding claims, wherein one of A and B is independently selected from

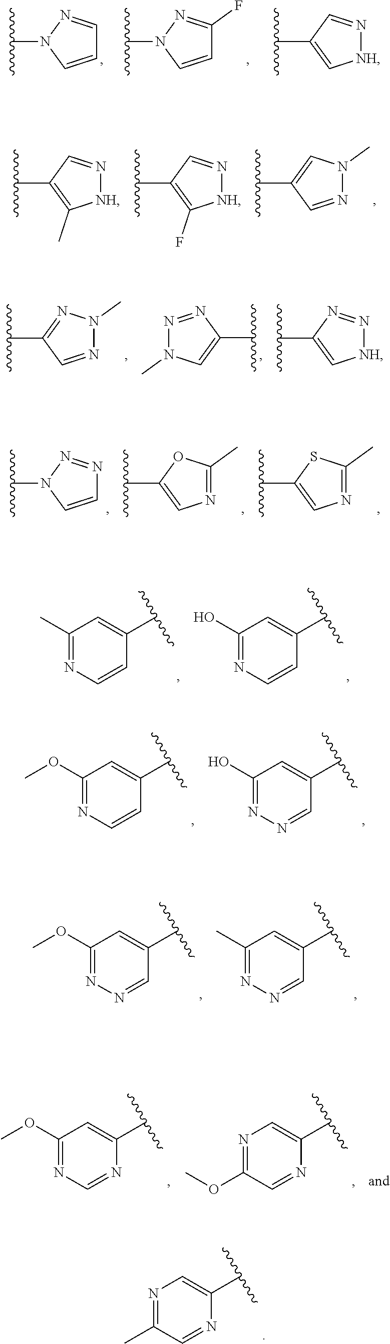

81. The compound of any one of the preceding claims, wherein one of A and B is independently selected from

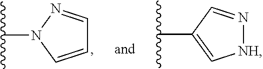 [SMILES: *n1cccn1 ; *c1cn[nH]c1]

82. The compound of any one of the preceding claims, wherein A is selected from

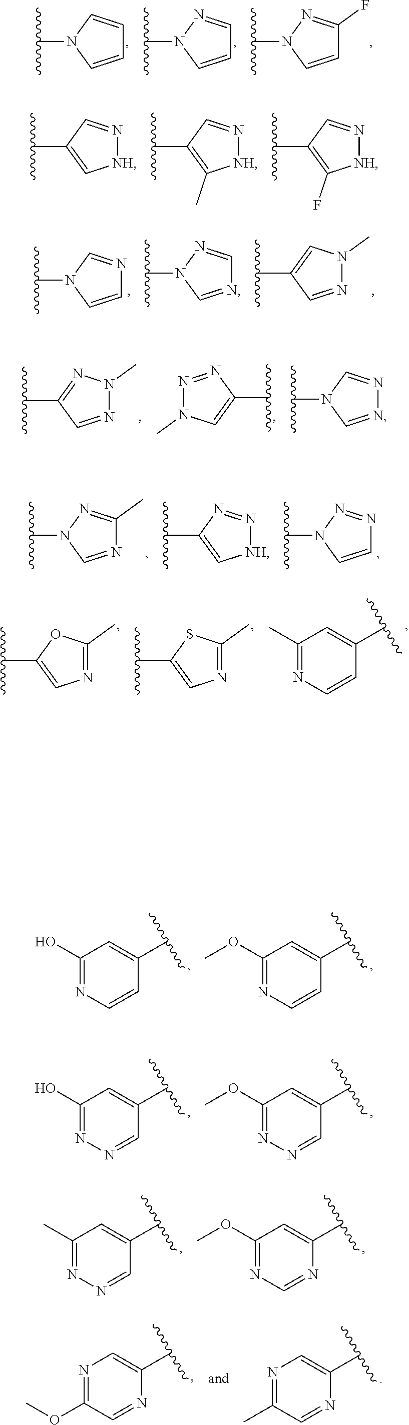

83. The compound of any one of the preceding claims wherein B is selected from

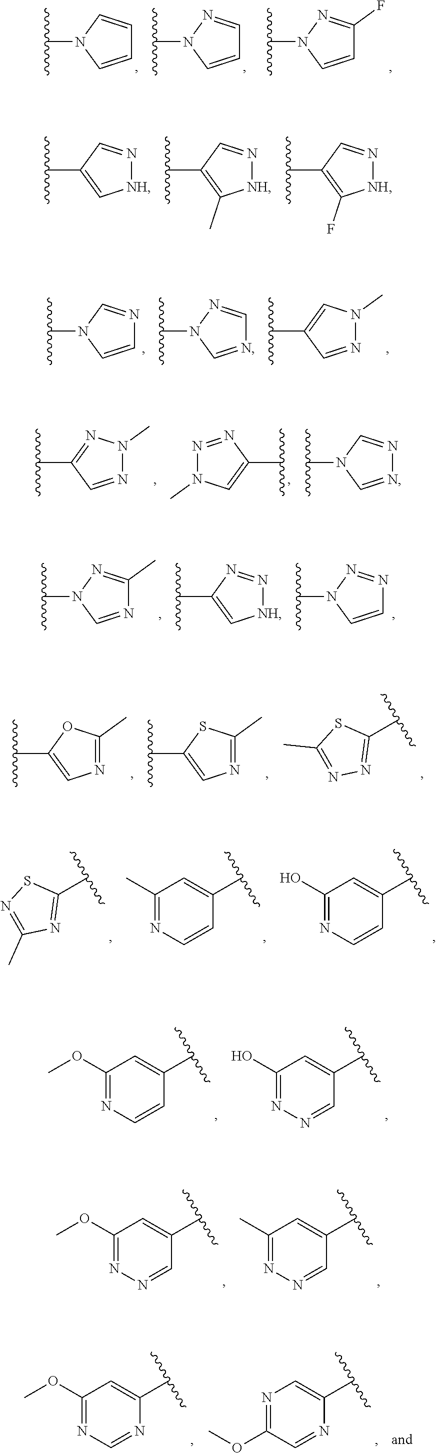

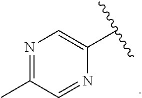 [SMILES: *c1cnc(C)cn1]

84. The compound ofany one of the preceding claims, wherein B is selected from

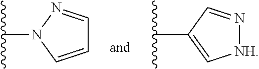 [SMILES: *n1cccn1 ; *c1cn[nH]c1]

85. The compound of any one of the preceding claims, wherein one of A and B is independently a monocyclic heterocyclyl or bicyclic heterocyclyl, each of which is optionally substituted with one or more R¹.

86. The compound of any one of the preceding claims, wherein one of A and B is independently a nitrogen-containing heterocyclyl optionally substituted with one or more R¹.

87. The compound of any one of the preceding claims, wherein one of A and B is independently a 4-10 membered heterocyclyl optionally substituted with one or more R¹.

88. The compound of any one of the preceding claims, wherein one of A and B is independently selected from 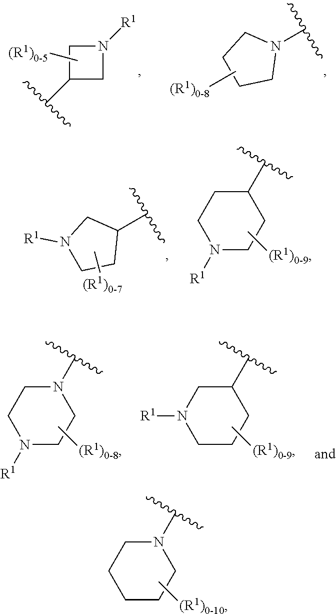
wherein R¹ is as described in claim 66.

89. The compound of any one of the preceding claims, wherein one of A and B is independently selected from 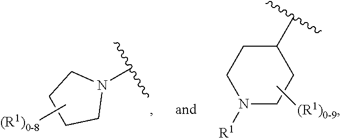
wherein R¹ is as described in claim 66.

90. The compound of any one of the preceding claims, wherein one of A and B is 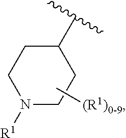
wherein R¹ is as described in claim 66.

91. The compound of any one of the preceding claims, wherein A is selected from 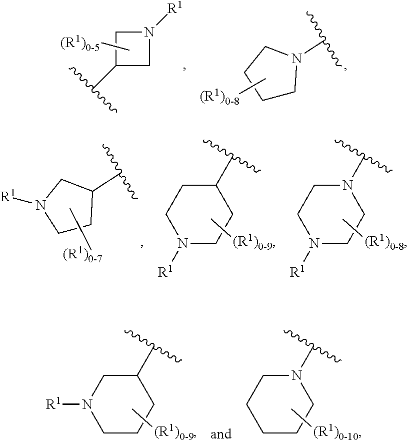
wherein R¹ is as described in claim 66.

92. The compound of any one of the preceding claims, wherein B is selected from 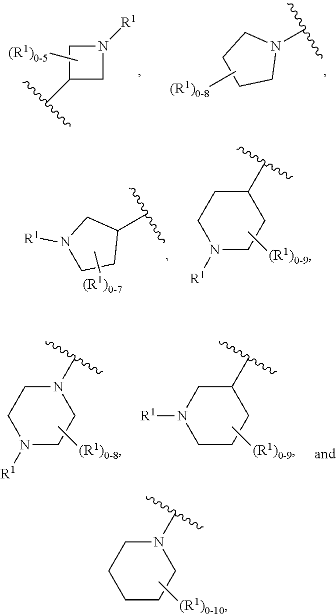
wherein R¹ is as described in claim 66.

93. The compound of any one of the preceding claims, wherein A is 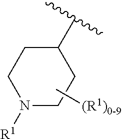
and R¹ is as described in claim 66.

94. The compound of any one of the preceding claims, wherein one of A and B is independently selected from

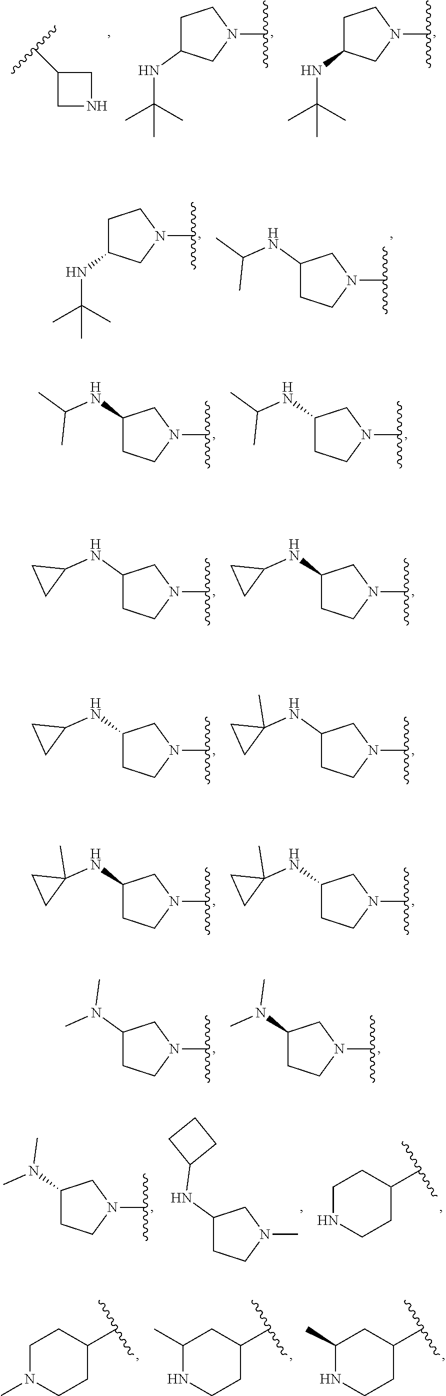

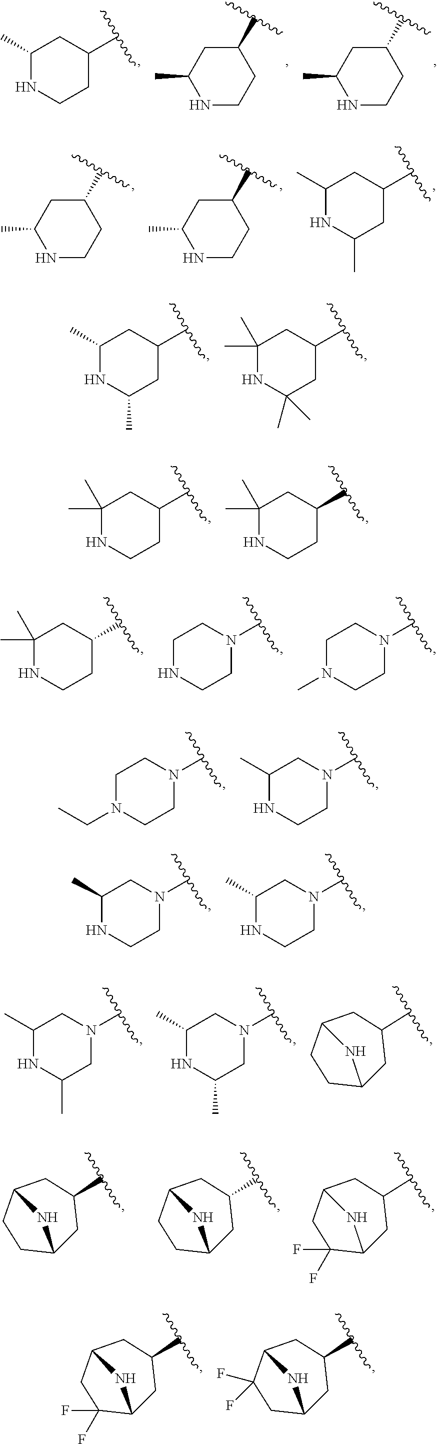

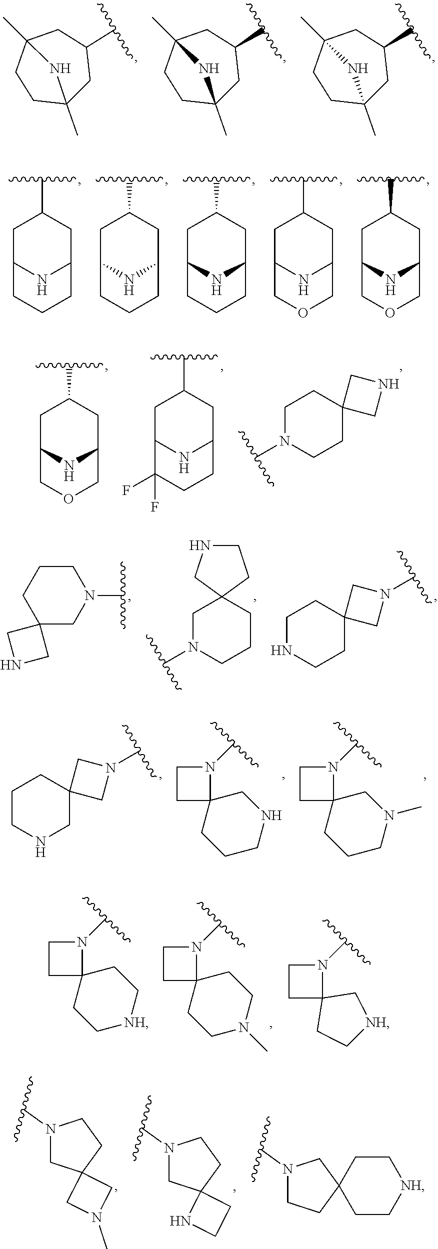

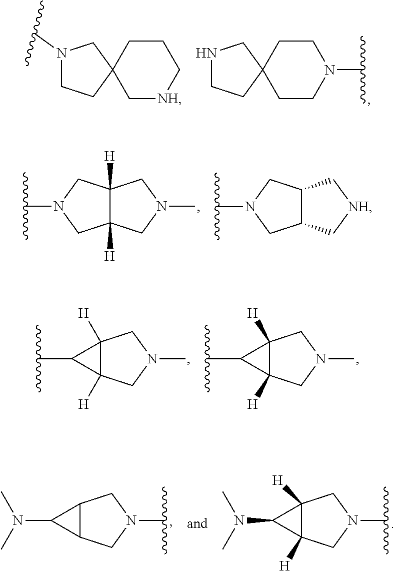

95. The compound of any one of the preceding claims, wherein one of A and B is independently selected from,

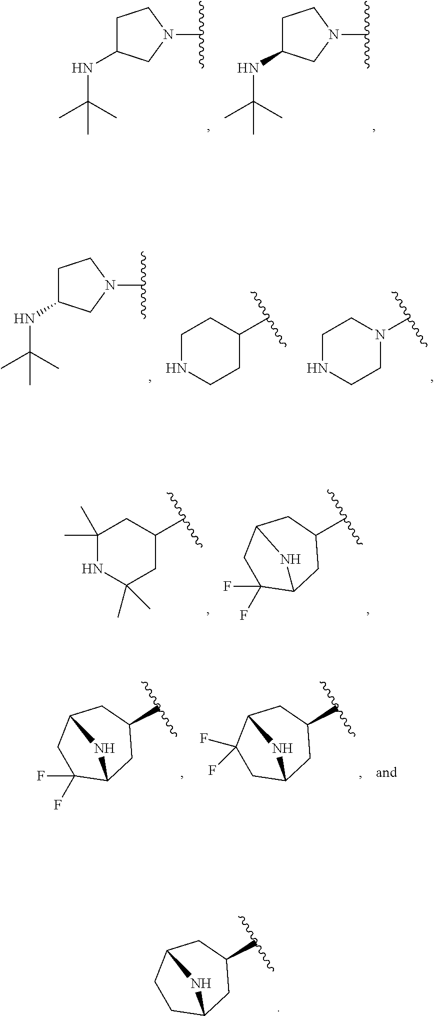

96. The compound of any one of the preceding claims, wherein one of A and B is independently selected from

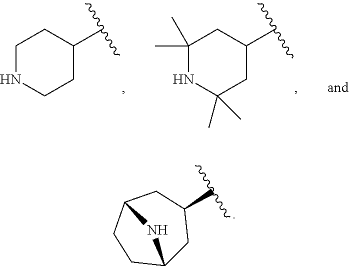 [SMILES: *C1CCNCC1 ; *C1CC(C)(C)NC(C)(C)C1 ; *[C@@H]1C[C@H]2CC[C@@H](C1)N2]

97. The compound of any one of the preceding claims, wherein A is selected from

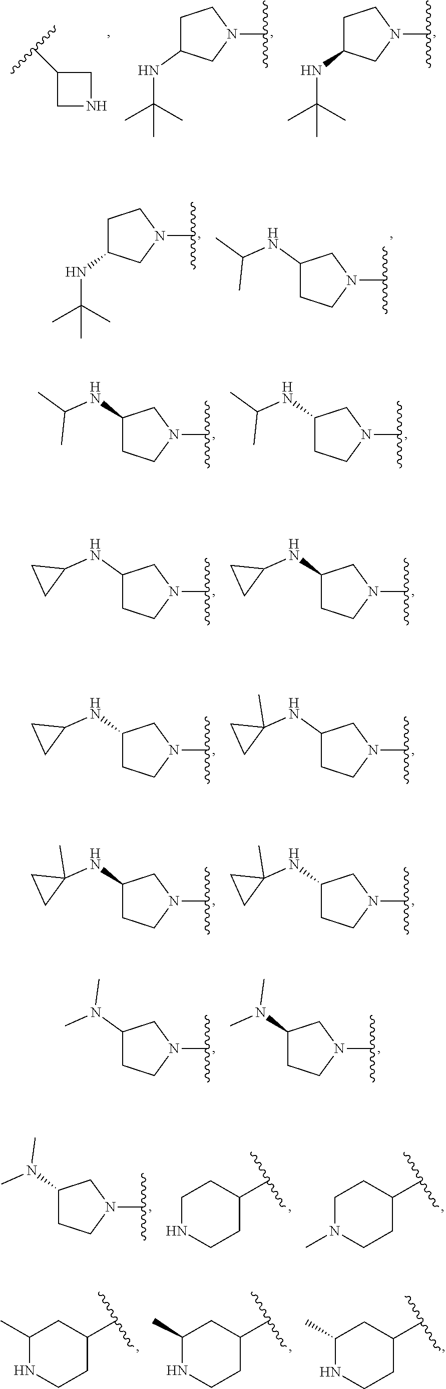

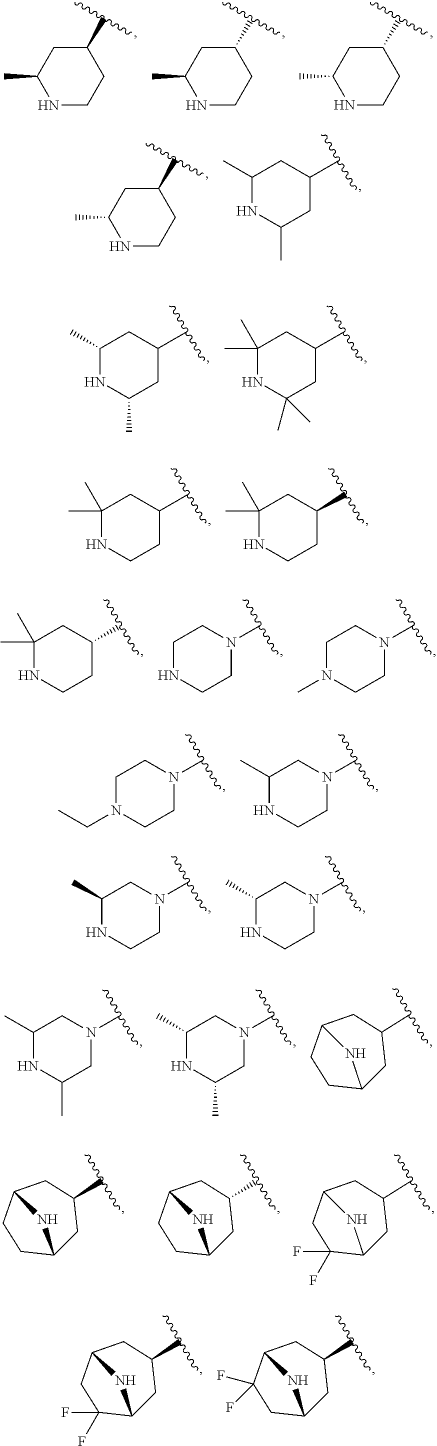

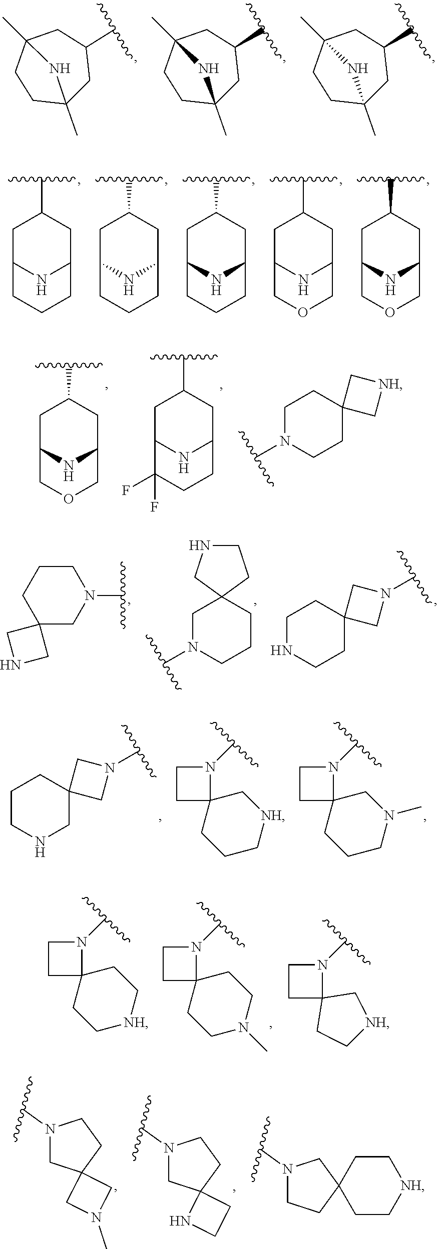

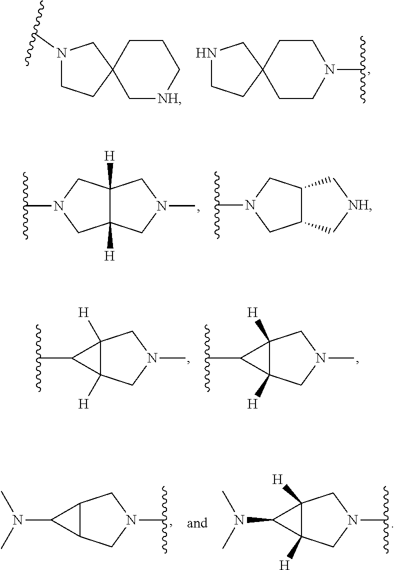

98. The compound of any one of the preceding claims, wherein B is selected from

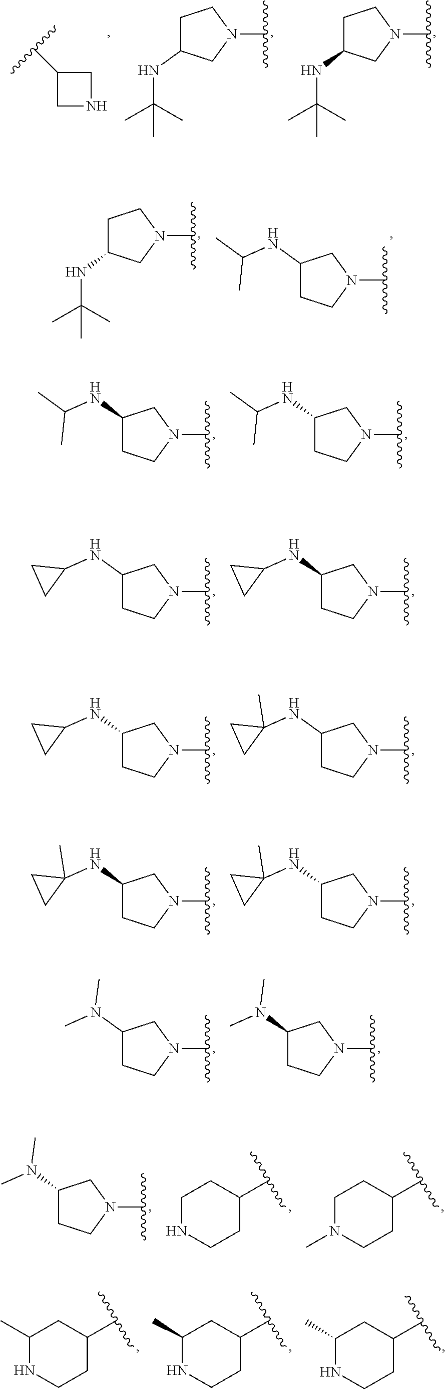

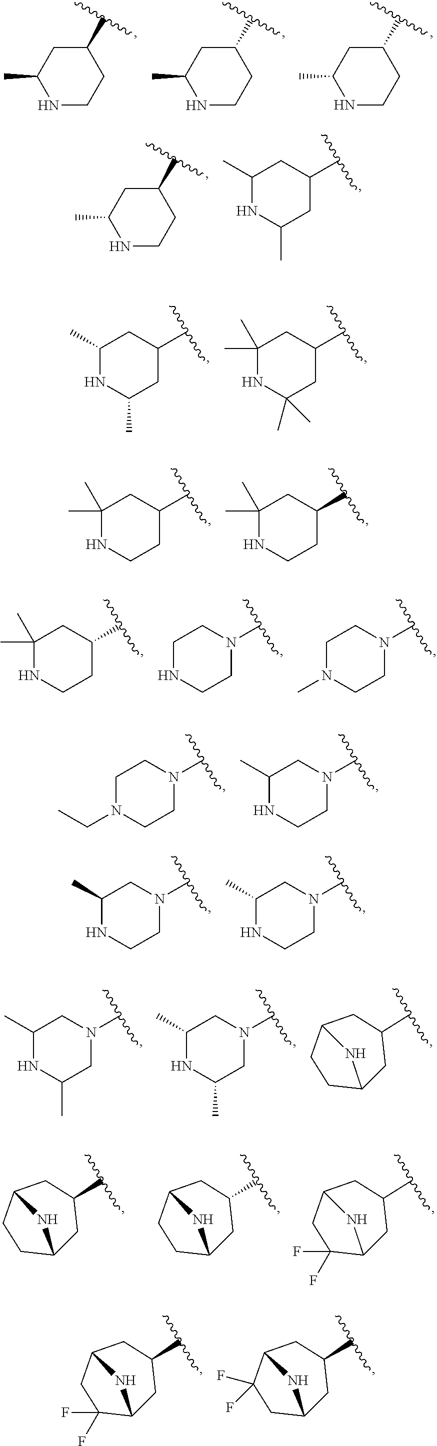

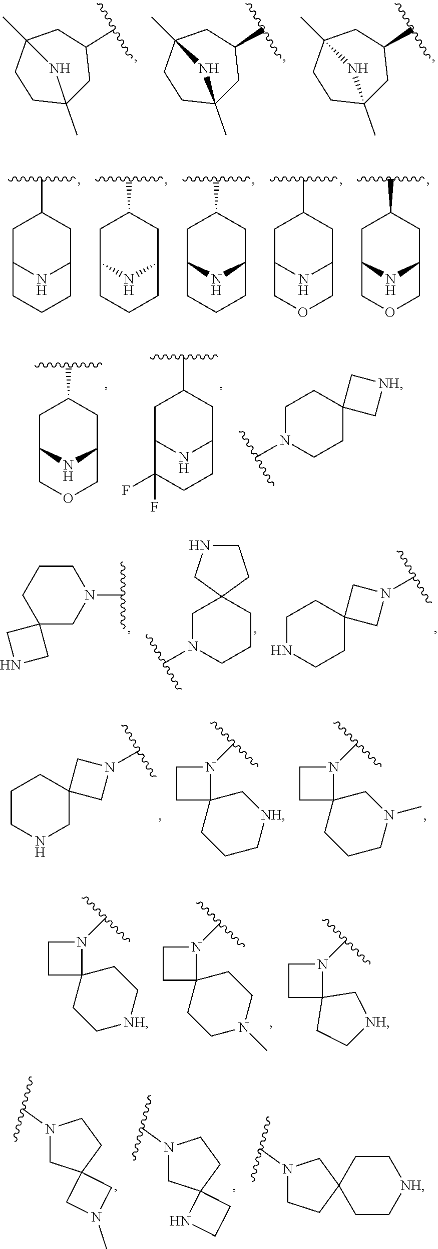

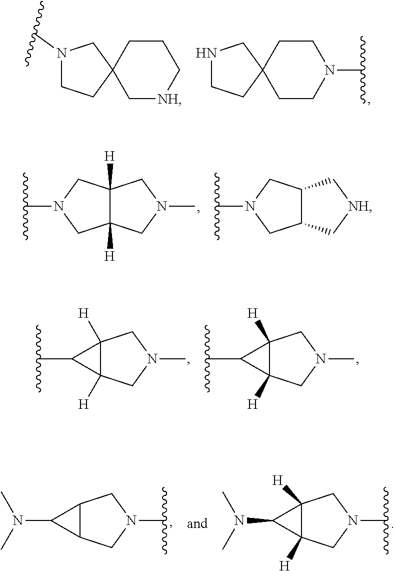

99. The compound of any one of the preceding claims, wherein A is selected from

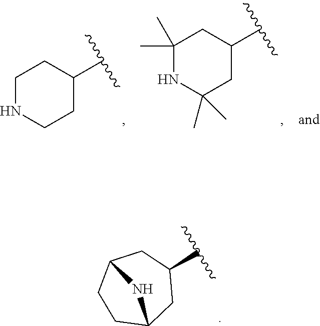

100. The compound of any one of the preceding claims, wherein L is absent, —O—, or —N(R³)—.

101. The compound of any one of the preceding claims, wherein L is absent.

102. The compound of any one of the preceding claims, wherein L is —O— or —N(R³)—.

103. The compound of any one of claims 1-36, wherein when L is —O— or —N(R³)—, A is heterocyclyl optionally substituted with one or more R¹.

104. The compound of any one of the preceding claims, wherein L is —N(CH₃)— or —N(H)—.

105. The compound of any one of the preceding claims, wherein L is —N(CH₃)—.

106. The compound of any one of the preceding claims, wherein each of M and P is independently C(R²).

107. The compound of any one of the preceding claims, wherein one of M and P is independently N.

108. The compound of any one of the preceding claims, wherein 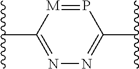
is selected from

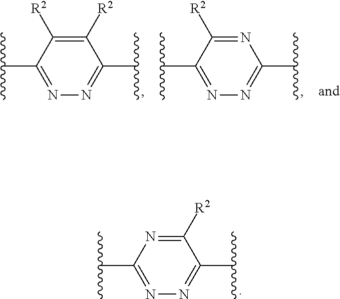

109. The compound of any one of the preceding claims, wherein one of X and Y is C(R^{5a})(R^{5b}) and the other of X and Y is N(R^{5c}).

110. The compound of claim 109, wherein R^{5a} and R^{5b}, together with the carbon atom to which they are attached, form an oxo group.

111. The compound of any one of the preceding claims, wherein X is N(R^{5c}) (e.g., NH) and Y is C(R^{5a})(R^{5b}) (e.g., C(O)).

112. The compound of any one of the preceding claims, wherein X is N and Y is C(R^{5a}) (e.g., C(OCH₃)).

113. The compound of any one of the preceding claims, wherein one of X and Y is C(O) and the other of X and Y is NH.

114. The compound of any one of the preceding claims, wherein 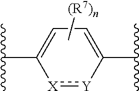
is selected from

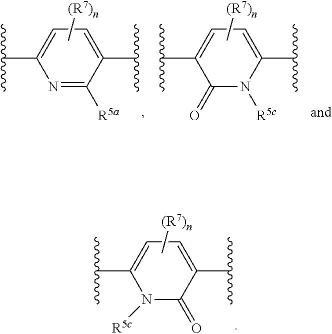

115. The compound of any one of the preceding claims, wherein Y is C(R^{5a}), and R^{5a} is selected from C₁-C₆-alkyl, C₁-C₆-haloalkyl, halo, cyano, —OR^{A}, and —NR^{B}R^{C}.

116. The compound of any one the preceding claims, wherein the compound is a compound of Formula (II-a): 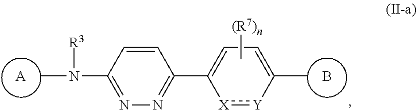
or a pharmaceutically acceptable salt, solvate, hydrate, tautomer, or stereoisomer thereof, wherein A, B, X, Y, R³, R⁷, n, and subvariables thereof are as described in claim 66.

117. The compound of any one the preceding claims, wherein the compound is a compound of Formula (II-b): 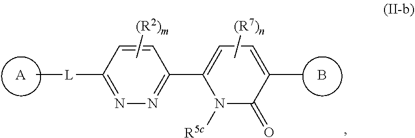
or a pharmaceutically acceptable salt, solvate, hydrate, tautomer, or stereoisomer thereof, wherein A, B, L, R², R^{Sc}, R⁷, m, n, and subvariables thereof are as described in claim 66.

118. The compound of any one the preceding claims, wherein the compound is a compound of Formula (II-c): 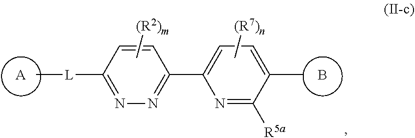
or a pharmaceutically acceptable salt, solvate, hydrate, tautomer, or stereoisomer thereof, wherein A, B, L, R², R^{5a}, R⁷, m, n, and subvariables thereof are as described in claim 66.

119. The compound of any one of the preceding claims, wherein the compound is a compound of Formula (II-d): 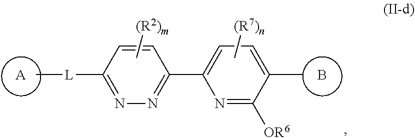
or a pharmaceutically acceptable salt, solvate, hydrate, tautomer, or stereoisomer thereof, wherein R⁶ is C₁-C₆-alkyl, C₂-C₆-alkenyl, C₂-C₆-alkynyl, C₁-C₆-heteroalkyl, C₁-C₆-haloalkyl, cycloalkyl, —C(O)R^{D}, or —C(O)OR^{D}; m is 0, 1, or 2; and A, B, L, R², R⁷, n, and subvariables thereof are as described in claim 66.

120. The compound of any one of the preceding claims, wherein:
A is a 5 or 6-membered nitrogen-containing heterocyclyl;
B is a 5 or 6-membered nitrogen-containing heteroaryl;
L is —O— or —N(R³)—;
X is N;
Y is OR⁶; and
R⁶ is C₁-C₆-alkyl, C₂-C₆-alkenyl, C₂-C₆-alkynyl, C₁-C₆-heteroalkyl, C₁-C₆-haloalkyl, cycloalkyl, —C(O)R^{D}, or —C(O)OR^{D}.

121. A compound of Formula (III): 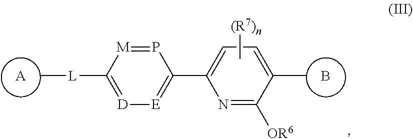
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L is absent, —O—, —C(O)—, —N(R³)—;
D, E, M, and P are each independently C(R²) or N, wherein at least one of D, E, M, and P is N;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸;
each R² is independently hydrogen, halo, cyano, C₁-C₆-alkyl, C₂-C₆-alkenyl, C₂-C₆-alkynyl, or —OR^{A};
each R³ is independently hydrogen, C₁-C₆-alkyl, or C₁-C₆-haloalkyl;
R⁶ is C₁-C₆-alkyl, C₂-C₆-alkenyl, C₂-C₆-alkynyl, C₁-C₆-heteroalkyl, C₁-C₆-haloalkyl, cycloalkyl, —C(O)R^{D}, or —C(O)OR^{D};
each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —S(O)ₓR^{D}, wherein alkyl, alkenyl, alkynyl, heteroalkyl, and haloalkyl are optionally substituted with one or more R⁹;
each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D};
R^{B} and R^{C} together with the atom to which they are attached form a 3-7-membered heterocyclyl or heteroaryl ring optionally substituted with one or more R¹⁰;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl;
R¹⁰ is C₁-C₆-alkyl or halo;
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
n is 0, 1, or 2; and
x is 1 or 2.

122. The compound of claim 121, wherein each of A and B is independently heteroaryl or heterocyclyl, each of which is optionally substituted with one or more R¹.

123. The compound of any one of the preceding claims, wherein one of A and B is independently a monocyclic heteroaryl or bicyclic heteroaryl, each of which is optionally substituted with one or more R¹.

124. The compound of any one of the preceding claims, wherein one of A and B is independently a monocyclic heteroaryl optionally substituted with one or more R¹.

125. The compound of any one of the preceding claims, wherein one of A and B is independently a nitrogen-containing heteroaryl optionally substituted with one or more R¹.

126. The compound of any one of the preceding claims, wherein one of A and B is independently a 5-6 membered heteroaryl optionally substituted with one or more R¹.

127. The compound of any one of the preceding claims, wherein one of A and B is independently selected from 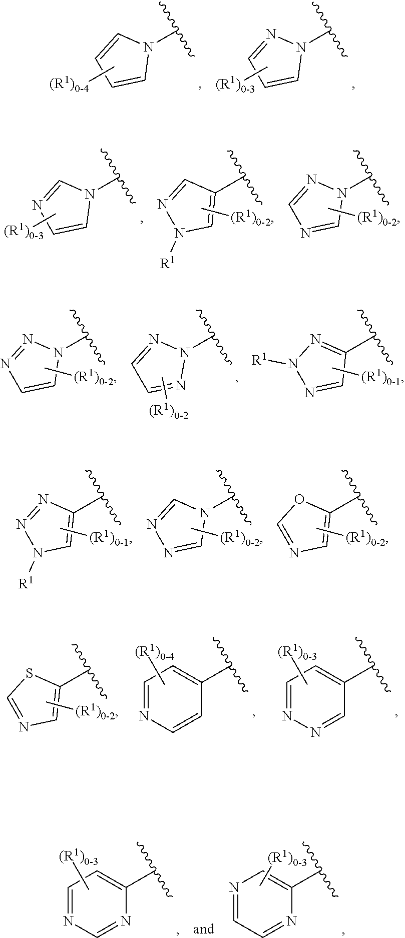
wherein R¹ is as described in claim 121.

128. The compound of any one of the preceding claims, wherein one of A and B is independently selected from 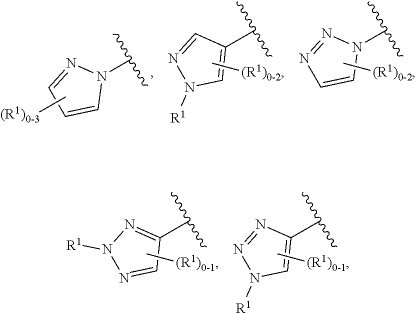
wherein R¹ is as described in claim 121.

129. The compound of any one of the preceding claims, wherein one of A and B is independently selected from 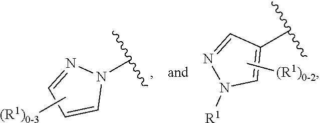
wherein R¹ is as described in claim 54.

130. The compound of any one of the preceding claims, wherein A is selected from 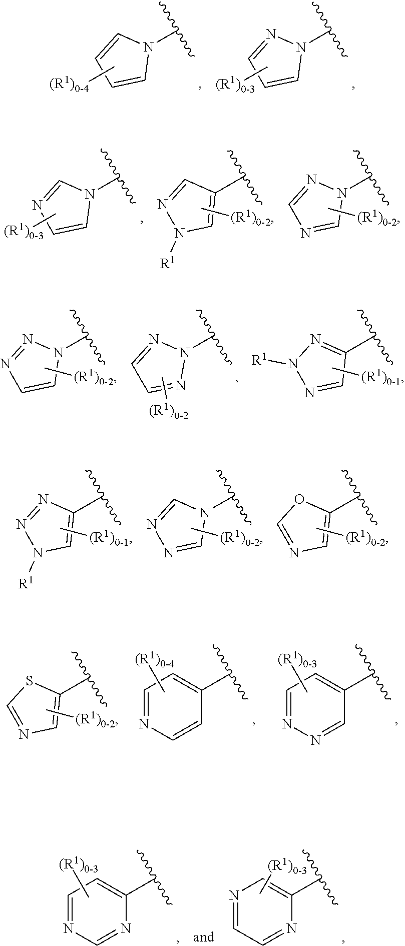
wherein R¹ is as described in claim 121.

131. The compound of any one of the preceding claims, wherein B is selected from 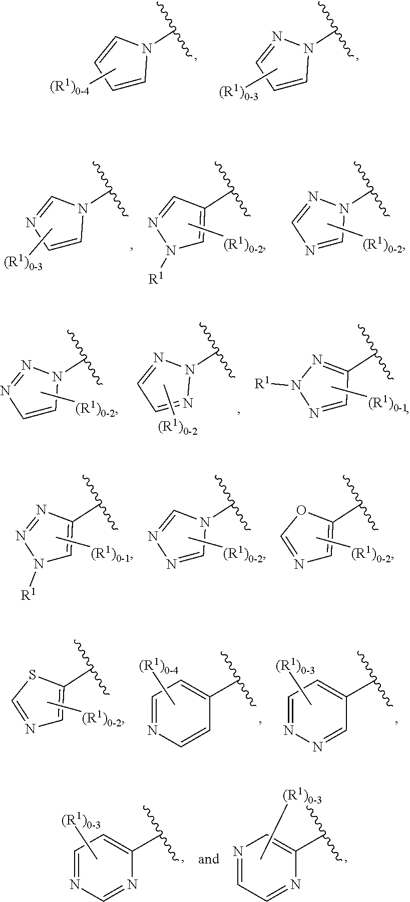
wherein R¹ is as described in claim 121.

132. The compound of any one of the preceding claims, B is selected from 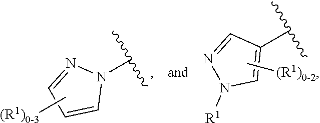
wherein R¹ is as described in claim 121.

133. The compound of any one of the preceding claims, wherein one of A and B is independently selected from

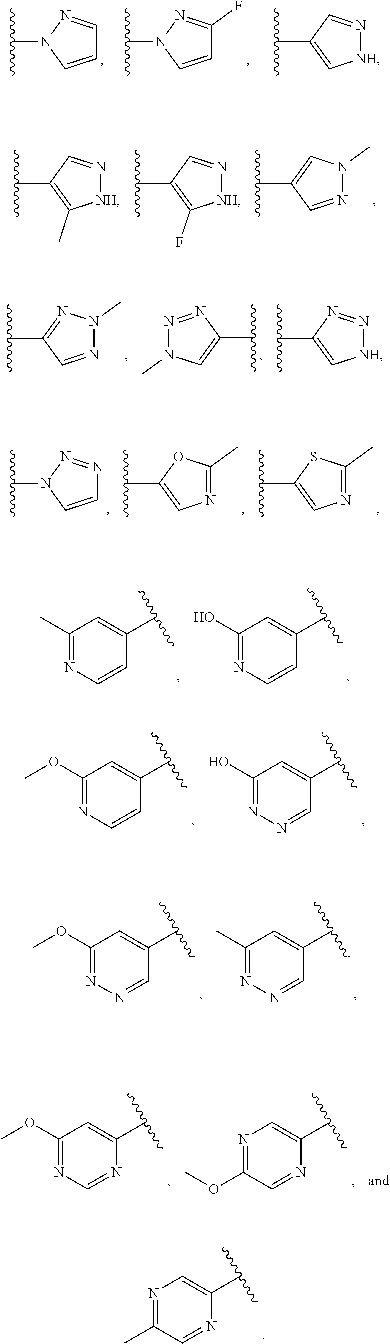

134. The compound of any one of the preceding claims, wherein one of A and B is independently selected from

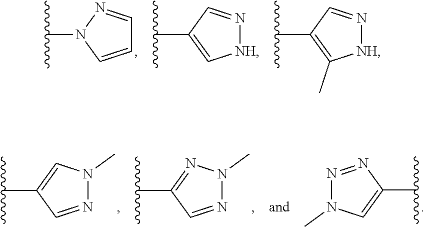

135. The compound of any one of the preceding claims, wherein one of A and B is independently selected from

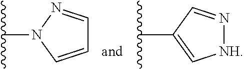 [SMILES: *n1cccn1 ; *c1cn[nH]c1]

136. The compound of any one of the preceding claims, wherein A is selected from

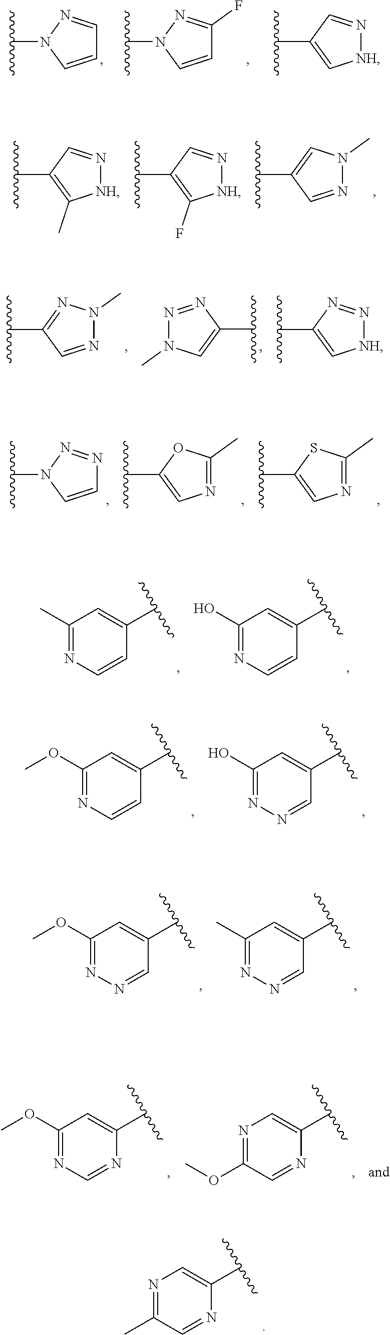

137. The compound of any one of the preceding claims, wherein B is selected from

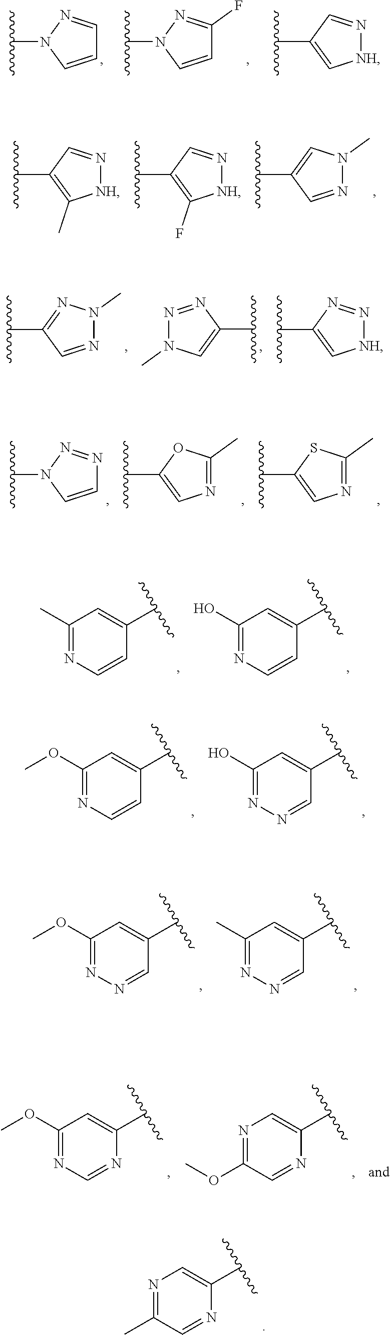

138. The compound of any one of the preceding claims, wherein B is selected from

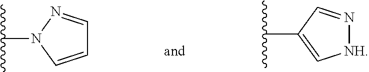 [SMILES: *n1cccn1 ; *c1cn[nH]c1]

139. The compound of any one of the preceding claims, wherein one of A and B is independently a monocyclic heterocyclyl or bicyclic heterocyclyl, each of which is optionally substituted with one or more R¹.

140. The compound of any one of the preceding claims, wherein one of A and B is independently a nitrogen-containing heterocyclyl optionally substituted with one or more R¹.

141. The compound of any one of the preceding claims, wherein one of A and B is independently a 4-10 membered heterocyclyl optionally substituted with one or more R¹.

142. The compound of any one of the preceding claims, wherein one of A and B is independently selected from 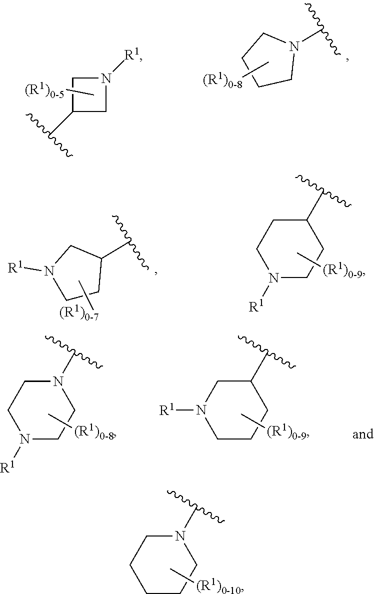
wherein R¹ is as described in claim 54.

143. The compound of any one of the preceding claims, wherein one of A and B is independently 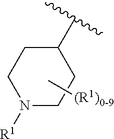
and R¹ is as described in claim 121.

144. The compound of any one of the preceding claims, wherein A is selected from 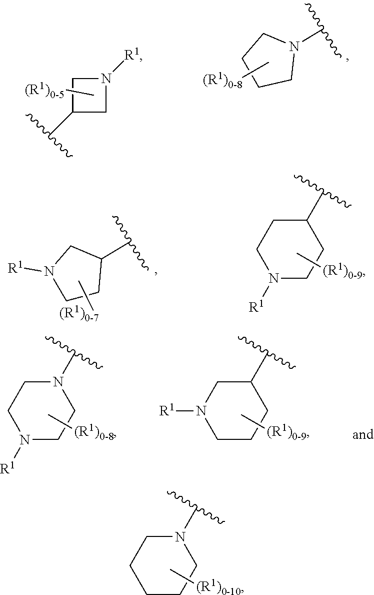
wherein R¹ is as described in claim 121.

145. The compound of any one of the preceding claims, wherein B is selected from 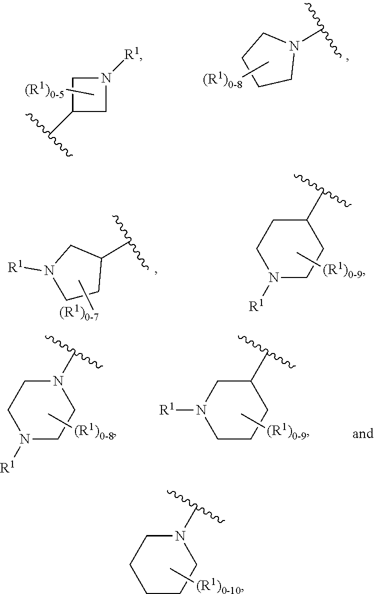
wherein R¹ is as described in claim 121.

146. The compound ofany one of the preceding claims, wherein A is 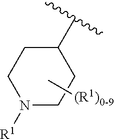
and R¹ is as described in claim 121.

147. The compound of any one of the preceding claims, wherein one of A and B is independently selected from

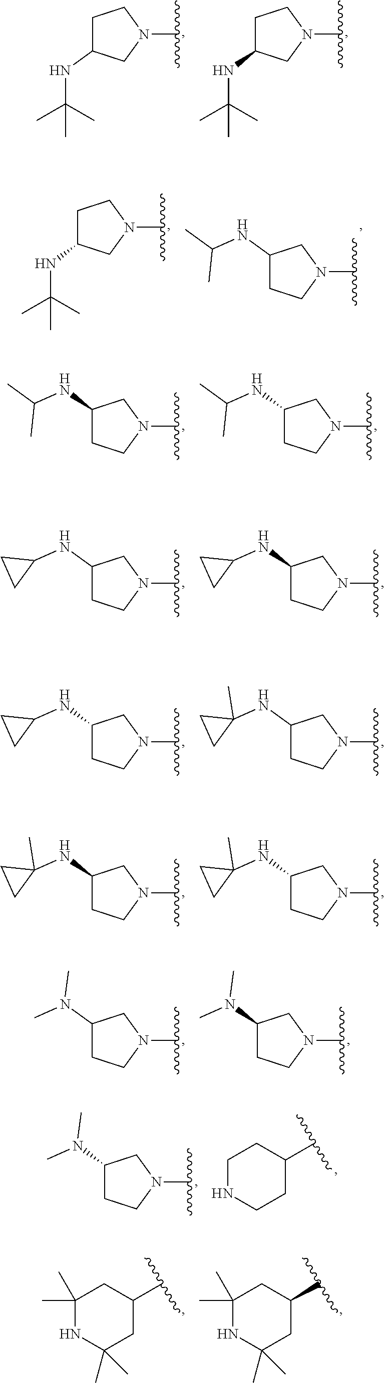

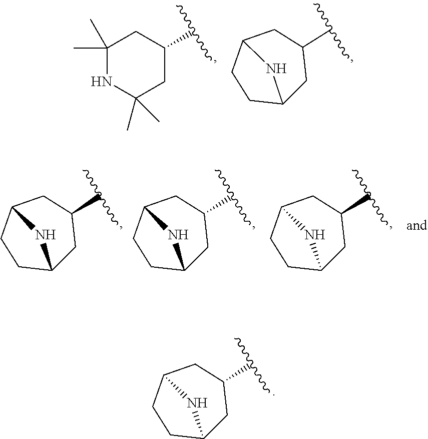

148. The compound of any one of the preceding claims, wherein one of A and B is independently selected from

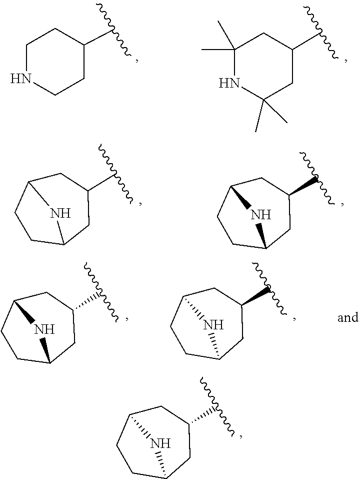

149. The compound of any one of the preceding claims, wherein A is selected from

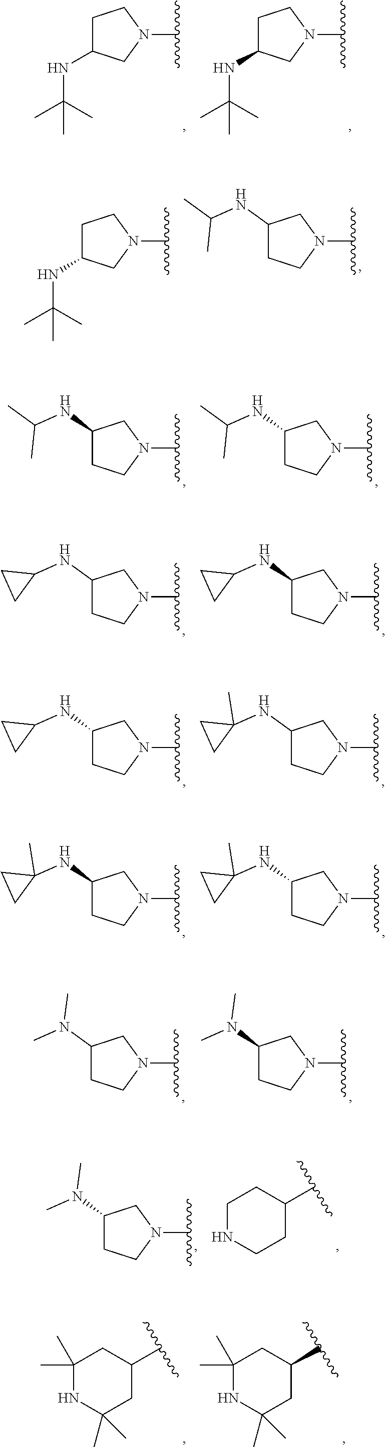

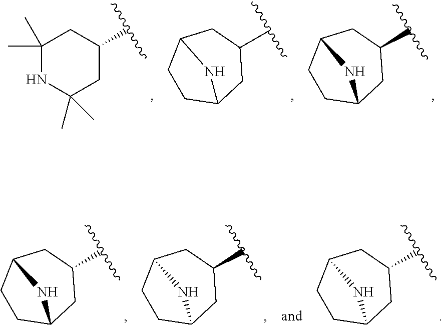

150. The compound of any one of the preceding claims, wherein B is selected from

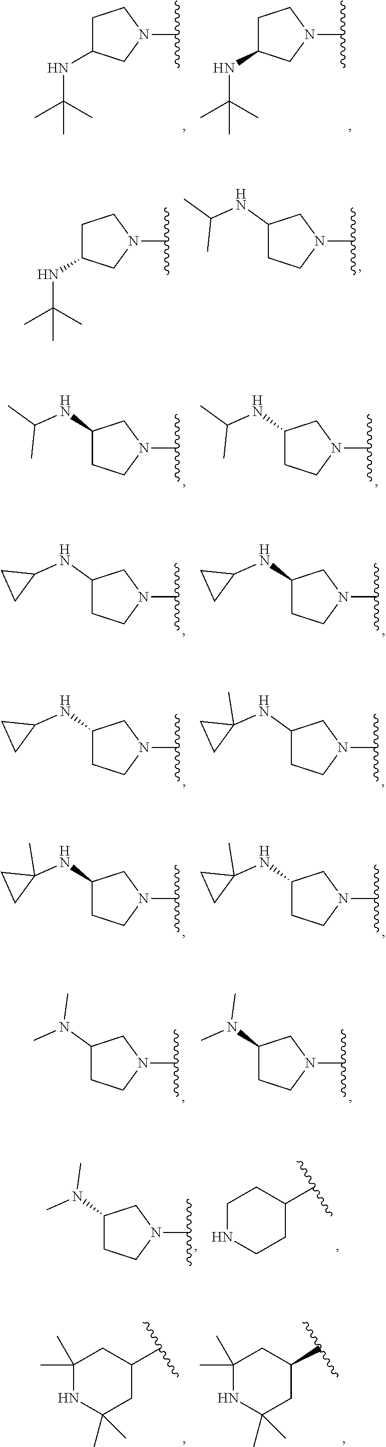

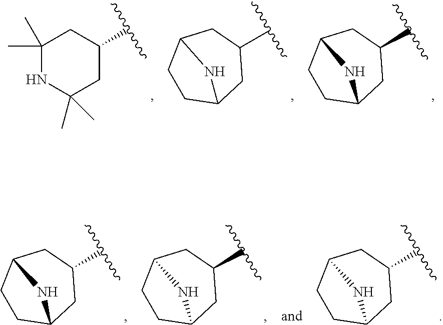

151. The compound of any one of the preceding claims, wherein A is selected from

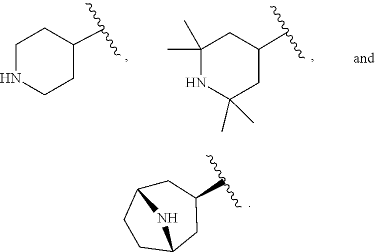 [SMILES: *C1CCNCC1 ; *C1CC(C)(C)NC(C)(C)C1 ; *[C@@H]1C[C@H]2CC[C@@H](C1)N2]

152. The compound of any one of the preceding claims, wherein L is absent, —O— or —N(R³)—.

153. The compound of claim 152, wherein A is heteterocyclyl optionally substituted with one or more R¹.

154. The compound of any one of the preceding claims, wherein L is O.

155. The compound of any one of the preceding claims, wherein L is —N(CH₃)— or —N(H)—.

156. The compound of any one of the preceding claims, wherein L is —N(CH₃)—.

157. The compound of any one of the preceding claims, wherein one of D and E is independently N.

158. The compound of any one of the preceding claims, wherein one of D and M is independently N.

159. The compound of any one of the preceding claims, wherein one of P and E is independently N.

160. The compound of any one of the preceding claims, wherein one of M and P is independently N.

161. The compound of any one of the preceding claims, wherein R⁶ is C₁-C₆-alkyl, C₁-C₆-heteroalkyl, C₁-C₆-haloalkyl, or cycloalkyl.

162. The compound of any one of the preceding claims, wherein R⁶ is C₁-C₆-alkyl (CH₃).

163. The compound of any one of the preceding claims, wherein the compound is a compound of Formula (III-a): 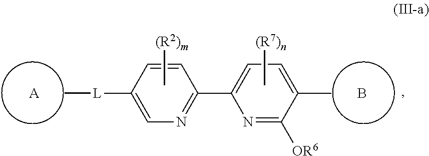
or a pharmaceutically acceptable salt, solvate, hydrate, tautomer, or stereoisomer thereof, wherein m is 0, 1, 2, or 3, and A, B, R², R⁶, R⁷, n, and subvariables thereof are as described in claim 121.

162. The compound of any one of the preceding claims, wherein the compound is a compound of Formula (III-b): 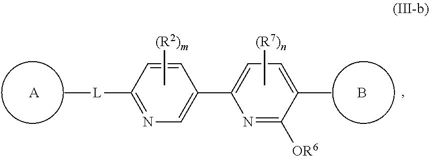
or a pharmaceutically acceptable salt, solvate, hydrate, tautomer, or stereoisomer thereof, wherein m is 0, 1, 2, or 3, and A, B, R², R⁶, R⁷, n, and subvariables thereof are as described in claim 121.

163. The compound of any one of claims 54-97, wherein the compound is a compound of Formula (III-c): 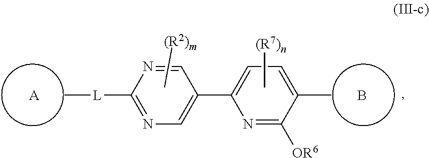
or a pharmaceutically acceptable salt, solvate, hydrate, tautomer, or stereoisomer thereof, wherein m is 0, 1, 2, or 3, and A, B, R², R⁶, R⁷, n, and subvariables thereof are as described in claim 121.

164. The compound of any one of the preceding claims, wherein the compound is a compound of Formula (III-d): 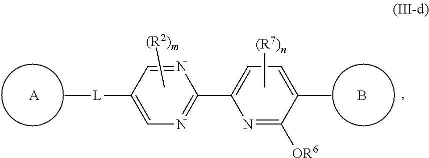
or a pharmaceutically acceptable salt, solvate, hydrate, tautomer, or stereoisomer thereof, wherein m is 0, 1, 2, or 3, and A, B, R², R⁶, R⁷, n, and subvariables thereof are as described in claim 54.

165. The compound of any one of the preceding claims, wherein the compound is a compound of Formula (III-e): 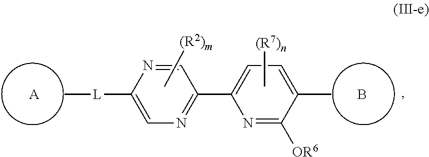
or a pharmaceutically acceptable salt, solvate, hydrate, tautomer, or stereoisomer thereof, wherein m is 0, 1, 2, or 3, and A, B, R², R⁶, R⁷, n, and subvariables thereof are as described in claim 121.

166. The compound of any one of the preceding claims, wherein the compound is a compound of Formula (III-f): 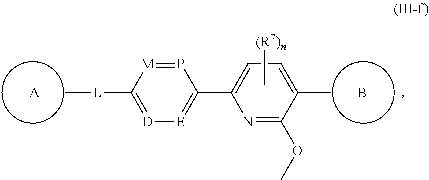
or a pharmaceutically acceptable salt, solvate, hydrate, tautomer, or stereoisomer thereof, wherein A, B, M, P, D, E, L, R⁷, n, and subvariables thereof are as described in claim 55.

167. The compound of any one of the preceding claims, wherein:
A is a 5 or 6-membered nitrogen-containing heterocyclyl;
B is a 5 or 6-membered nitrogen-containing heteroaryl; and
L is —O— or —N(R³)—.

168. The compound of any one of the preceding claims, wherein the compound is a compound of Formula (III-g): 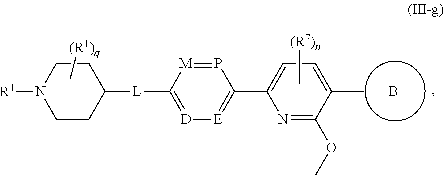
or a pharmaceutically acceptable salt, solvate, hydrate, tautomer, or stereoisomer thereof, wherein B is a 5-membered nitrogen-containing heteroaryl optionally substituted with one or more R¹, q is an integer between 0 and 9, and M, P, D, E, L, R⁷, n, and subvariables thereof are as described in claim 121.

169. The compound of any one of the preceding claims, wherein the compound is selected from any one of the compounds shown in any one of Tables 1, 2, or 3, or a pharmaceutically acceptable salt, solvate, hydrate, tautomer, or stereoisomer thereof.

170. The compound of any one of the preceding claims, wherein the compound is selected from any one of the compounds shown in Table 1 or a pharmaceutically acceptable salt, solvate, hydrate, tautomer, or stereoisomer thereof.

171. The compound of any one of the preceding claims, wherein the compound is selected from any one of the compounds shown in Table 2 or a pharmaceutically acceptable salt, solvate, hydrate, tautomer, or stereoisomer thereof.

172. The compound of any one of the preceding claims, wherein the compound is selected from any one of the compounds shown in Table 3 or a pharmaceutically acceptable salt, solvate, hydrate, tautomer, or stereoisomer thereof.

173. A pharmaceutical composition comprising a compound of any one of the preceding claims and a pharmaceutically acceptable excipient.

174. The compound of any one of claims 1-172, or the pharmaceutical composition of claim 173, wherein the compound alters a target nucleic acid (e.g., an RNA, e.g., a pre-mRNA).

175. The compound of any one of claims 1-172, or the pharmaceutical composition of claim 173, wherein the compound binds to a target nucleic acid (e.g., an RNA, e.g., a pre-mRNA).

176. The compound of any one of claims 1-172, or the pharmaceutical composition of claim 173, wherein the compound stabilizes a target nucleic acid (e.g., an RNA, e.g., a pre-mRNA).

177. The compound of any one of claims 1-172, or the pharmaceutical composition of claim 173, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

178. The compound of any one of claims 1-172, or the pharmaceutical composition of claim 173, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

179. A method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), or (III) according to any one of claims 1-172, comprising contacting the nucleic acid (e.g., a DNA, RNA, e.g., a pre-mRNA) with a compound of Formula (I), (II), or (III).

180. The method of claim 179, wherein the component of a spliceosome is recruited to the nucleic acid in the presence of the compound of Formula (I), (II), or (III).

181. A method of altering the conformation of a nucleic acid (e.g., a DNA, RNA, e.g., a pre-mRNA) comprising contacting the nucleic acid with a compound of Formula (I), (II), or (III), according to any one of claims 1-172 or the pharmaceutical composition of claim 173.

182. The method of claim 181, wherein the altering comprises forming a bulge in the nucleic acid.

183. The method of claim 181, wherein the altering comprises stabilizing a bulge in the nucleic acid.

184. The method of claim 181, wherein the altering comprises reducing a bulge in the nucleic acid.

185. The method of any one of any one of claims 181-184, wherein the nucleic acid comprises a splice site.

186. A method for treating a disease or disorder in a subject comprising administering to the subject a compound of Formula (I), (II), or (III), according to any one of claims 1-172 or the pharmaceutical composition of claim 173.

187. The method of claim 186, wherein the disease or disorder comprises a proliferative disease (e.g., cancer, a benign neoplasm, or angiogenesis).

188. The method of any one of claims 186-187, wherein the disease or disorder comprises cancer.

189. The method of claim 188, wherein the cancer is selected form adenoid cystic carcinoma, colorectal cancer, leukemia, lung cancer, prostate cancer, breast cancer, or ovarian cancer.

190. The method of claim 189, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

191. The method of claim 190, wherein the disease or disorder comprises a neurological disease or disorder.

192. The method of any one of claims 190-191, wherein the disease or disorder comprises Huntington's disease.
